(19) United States
(12) Patent Application Publication
(10) Pub. No.: US 2025/0340659 A1
(43) Pub. Date: Nov. 6, 2025

(54) THERAPEUTIC ANTIBODIES

(71) Applicant: PETMEDIX LTD., Cambridge (GB)

(72) Inventor: Roberto BANDIERA, Cambridge (GB)

(21) Appl. No.: 18/837,256

(22) PCT Filed: Feb. 8, 2023
(86) PCT No.: PCT/GB2023/050281

§ 371 Date: Aug. 9, 2024

(30) Foreign Application Priority Data
Feb. 9, 2022 (GB) 2201674.5
Jun. 28, 2022 (GB) 2209454.4

(51) Int. Cl. C07K 16/28; A61K 39/00; G01N 33/68

(52) U.S. Cl. CPC C07K 16/2875; G01N 33/6854; A61K 2039/505; C07K 2317/24; C07K 2317/52; C07K 2317/732; C07K 2317/734; C07K 2317/76; C07K 2317/92; C07K 2317/94; G01N 2333/70578

(57) ABSTRACT

The invention relates to antibodies which modulate the OX40 signalling pathway to treat inflammatory diseases in companion animals, for example atopic dermatitis (AD) and/or eczema. Related methods and uses are also described.

### INTRODUCTION

The present invention relates to antibodies which modulate the OX40 signalling pathway to treat inflammatory diseases in companion animals.

Atopic dermatitis (AD) and/or eczema, characterised by chronic, dry, itchy, red skin, is a significant problem in dogs, effecting 10-15% of pet dogs.

Cytopoint is an existing treatment for Atopic Dermatitis in dogs that has a recommended minimum dose of 1 mg kg⁻¹, by injection, once a month. Cytopoint is an anti-IL31 Ab (described for example in WO2013/011407A1 and WO2019/177697), specifically intended to treat the itch (pruritus) associated with atopic dermatitis. However, there are at least one third of canine AD patients that do not satisfactorily respond to Cytopoint, and in some instances, efficacy can decrease following the first injection (see, for example “CVMP assessment report for CYTOPOINT EMA/118401/2017 (EMEA/V/C/003939/0000)”, and World Association for Veterinary Dermatology, Cytopoint roundtable May 2017, https://wavd.org/wp-content/uploads/cytopoint-roundtable-2017-05.pd). The most common side effects of Cytopoint (which may affect up to 1 in 1,000 animals) are allergic reactions such as anaphylaxis, facial oedema and urticaria. Cytopoint must not be given to dogs weighing less than 3 kg. (https://www.ema.europa.eu/en/documents/product-information/cytopoint-epar-product-information_en.pdf).

There is a need for improved treatments as well as for drugs that treat the underlying cause of the disease rather than the symptoms. In particular, there is a need for a treatment that is safe, has a long duration of action, and has efficacy to cover a wider spectrum of patients, particularly non-responders.

Advantageously, targeting OX40/OX40L which are upstream in the inflammatory cascade, offers the possibility to modulate multiple cytokines simultaneously.

Canine OX40L is described in U.S. Pat. No. 10,196,435. The protein sequence of canine OX40 has not been reported in the scientific or patent literature to date.

A monoclonal antibody (7D6) that binds feline CD134 (OX40) and its effect on the feline immunodeficiency virus is described in Willett et al, Journal of Virology, 81 (18), 2007, pages 9665-9679.

The use of an antibody to OX40L or OX40 in the treatment of immune-regulated diseases, such as atopic dermatitis, in companion animals (e.g. dogs) has not been shown before.

### STATEMENTS OF INVENTION

In a first aspect, the invention relates to an antibody or fragment thereof that specifically binds to companion animal OX40L or to companion animal OX40, wherein the antibody is selected from one of the following antibodies:
an antibody comprising a HC CDR1 comprising SEQ ID No: 575 or a sequence with at least 60%, 70%, 80%, 90% or 95% sequence identity thereto, a HC CDR2 comprising SEQ ID No: 576 or a sequence with at least 60%, 70%, 80%, 90% or 95% sequence identity thereto, a HC CDR3 comprising SEQ ID No: 577 or a sequence with at least 60%, 70%, 80%, 90% or 95% sequence identity thereto, a LC CDR1 comprising SEQ ID No: 578 or a sequence with at least 60%, 70%, 80%, 90% or 95% sequence identity thereto, a LC CDR2 comprising SEQ ID No: 579 or a sequence with at least 60%, 70%, 80%, 90% or 95% sequence identity thereto and a LC CDR3 comprising SEQ ID No: 580 or a sequence with at least 60%, 70%, 80%, 90% or 95% sequence identity thereto;
an antibody comprising a HC CDR1 comprising SEQ ID No: 741 or a sequence with at least 60%, 70%, 80%, 90% or 95% sequence identity thereto, a HC CDR2 comprising SEQ ID No: 742 or a sequence with at least 60%, 70%, 80%, 90% or 95% sequence identity thereto, a HC CDR3 comprising SEQ ID No: 743 or a sequence with at least 60%, 70%, 80%, 90% or 95% sequence identity thereto, a LC CDR1 comprising SEQ ID No: 744 or a sequence with at least 60%, 70%, 80%, 90% or 95% sequence identity thereto, a LC CDR2 comprising SEQ ID No: 745 or a sequence with at least 60%, 70%, 80%, 90% or 95% sequence identity thereto and a LC CDR3 comprising SEQ ID No: 746 or a sequence with at least 60%, 70%, 80%, 90% or 95% sequence identity thereto;
an antibody comprising a HC CDR1 comprising SEQ ID No: 275 or a sequence with at least 60%, 70%, 80%, 90% or 95% sequence identity thereto, a HC CDR2 comprising SEQ ID No: 276 or a sequence with at least 60%, 70%, 80%, 90% or 95% sequence identity thereto, a HC CDR3 comprising SEQ ID No: 277 or a sequence with at least 60%, 70%, 80%, 90% or 95% sequence identity thereto, a LC CDR1 comprising SEQ ID No: 278 or a sequence with at least 60%, 70%, 80%, 90% or 95% sequence identity thereto, a LC CDR2 comprising SEQ ID No: 279 or a sequence with at least 60%, 70%, 80%, 90% or 95% sequence identity thereto and a LC CDR3 comprising SEQ ID No: 280 or a sequence with at least 60%, 70%, 80%, 90% or 95% sequence identity thereto;
an antibody comprising a HC CDR1 comprising SEQ ID No: 395 or a sequence with at least 60%, 70%, 80%, 90% or 95% sequence identity thereto, a HC CDR2 comprising SEQ ID No: 396 or a sequence with at least 60%, 70%, 80%, 90% or 95% sequence identity thereto, a HC CDR3 comprising SEQ ID No: 397 or a sequence with at least 60%, 70%, 80%, 90% or 95% sequence identity thereto, a LC CDR1 comprising SEQ ID No: 398 or a sequence with at least 60%, 70%, 80%, 90% or 95% sequence identity thereto, a LC CDR2 comprising SEQ ID No: 399 or a sequence with at least 60%, 70%, 80%, 90% or 95% sequence identity thereto and a LC CDR3 comprising SEQ ID No: 400 or a sequence with at least 60%, 70%, 80%, 90% or 95% sequence identity thereto
an antibody comprising a HC CDR1 comprising SEQ ID No: 15 or a sequence with at least 60%, 70%, 80%, 90% or 95% sequence identity thereto, a HC CDR2 comprising SEQ ID No: 16 or a sequence with at least 60%, 70%, 80%, 90% or 95% sequence identity thereto, a HC CDR3 comprising SEQ ID No: 17 or a sequence with at least 60%, 70%, 80%, 90% or 95% sequence identity thereto, a LC CDR1 comprising SEQ ID No: 18 or a sequence with at least 60%, 70%, 80%, 90% or 95% sequence identity thereto, a LC CDR2 comprising SEQ ID No: 19 or a sequence with at least 60%, 70%, 80%, 90% or 95% sequence identity thereto and a LC CDR3 comprising SEQ ID No: 20 or a sequence with at least 60%, 70%, 80%, 90% or 95% sequence identity thereto
an antibody comprising a HC CDR1 comprising SEQ ID No: 45 or a sequence with at least 60%, 70%, 80%, 90% or 95% sequence identity thereto, a HC CDR2 comprising SEQ ID No: 46 or a sequence with at least 60%, 70%, 80%, 90% or 95% sequence identity thereto, a HC CDR3 comprising SEQ ID No: 47 or a sequence with at least 60%, 70%, 80%, 90% or 95% sequence identity thereto, a LC CDR1 comprising SEQ ID No: 48 or a sequence with at least 60%, 70%, 80%, 90% or 95% sequence identity thereto, a LC CDR2 comprising SEQ ID No: 49 or a sequence with at least 60%, 70%, 80%, 90% or 95% sequence identity thereto and a LC CDR3 comprising SEQ ID No: 50 or a sequence with at least 60%, 70%, 80%, 90% or 95% sequence identity thereto
an antibody comprising a HC CDR1 comprising SEQ ID No: 65 or a sequence with at least 60%, 70%, 80%, 90% or 95% sequence identity thereto, a HC CDR2 comprising SEQ ID No: 66 or a sequence with at least 60%, 70%, 80%, 90% or 95% sequence identity thereto, a HC CDR3 comprising SEQ ID No: 67 or a sequence with at least 60%, 70%, 80%, 90% or 95% sequence identity thereto, a LC CDR1 comprising SEQ ID No: 68 or a sequence with at least 60%, 70%, 80%, 90% or 95% sequence identity thereto, a LC CDR2 comprising SEQ ID No: 69 or a sequence with at least 60%, 70%, 80%, 90% or 95% sequence identity thereto and a LC CDR3 comprising SEQ ID No: 70 or a sequence with at least 60%, 70%, 80%, 90% or 95% sequence identity thereto;
an antibody comprising a HC CDR1 comprising SEQ ID No: 115 or a sequence with at least 60%, 70%, 80%, 90% or 95% sequence identity thereto, a HC CDR2 comprising SEQ ID No: 116 or a sequence with at least 60%, 70%, 80%, 90% or 95% sequence identity thereto, a HC CDR3 comprising SEQ ID No: 117 or a sequence with at least 60%, 70%, 80%, 90% or 95% sequence identity thereto, a LC CDR1 comprising SEQ ID No: 118 or a sequence with at least 60%, 70%, 80%, 90% or 95% sequence identity thereto, a LC CDR2 comprising SEQ ID No: 119 or a sequence with at least 60%, 70%, 80%, 90% or 95% sequence identity thereto and a LC CDR3 comprising SEQ ID No: 120 or a sequence with at least 60%, 70%, 80%, 90% or 95% sequence identity thereto;
an antibody comprising a HC CDR1 comprising SEQ ID No: 155 or a sequence with at least 60%, 70%, 80%, 90% or 95% sequence identity thereto, a HC CDR2 comprising SEQ ID No: 156 or a sequence with at least 60%, 70%, 80%, 90% or 95% sequence identity thereto, a HC CDR3 comprising SEQ ID No: 157 or a sequence with at least 60%, 70%, 80%, 90% or 95% sequence identity thereto, a LC CDR1 comprising SEQ ID No: 158 or a sequence with at least 60%, 70%, 80%, 90% or 95% sequence identity thereto, a LC CDR2 comprising SEQ ID No: 159 or a sequence with at least 60%, 70%, 80%, 90% or 95% sequence identity thereto and a LC CDR3 comprising SEQ ID No: 160 or a sequence with at least 60%, 70%, 80%, 90% or 95% sequence identity thereto;
an antibody comprising a HC CDR1 comprising SEQ ID No: 235 or a sequence with at least 60%, 70%, 80%, 90% or 95% sequence identity thereto, a HC CDR2 comprising SEQ ID No: 236 or a sequence with at least 60%, 70%, 80%, 90% or 95% sequence identity thereto, a HC CDR3 comprising SEQ ID No: 237 or a sequence with at least 60%, 70%, 80%, 90% or 95% sequence identity thereto, a LC CDR1 comprising SEQ ID No: 238 or a sequence with at least 60%, 70%, 80%, 90% or 95% sequence identity thereto, a LC CDR2 comprising SEQ ID No: 239 or a sequence with at least 60%, 70%, 80%, 90% or 95% sequence identity thereto and a LC CDR3 comprising SEQ ID No: 240 or a sequence with at least 60%, 70%, 80%, 90% or 95% sequence identity thereto;
an antibody comprising a HC CDR1 comprising SEQ ID No: 285 or a sequence with at least 60%, 70%, 80%, 90% or 95% sequence identity thereto, a HC CDR2 comprising SEQ ID No: 286 or a sequence with at least 60%, 70%, 80%, 90% or 95% sequence identity thereto, a HC CDR3 comprising SEQ ID No: 287 or a sequence with at least 60%, 70%, 80%, 90% or 95% sequence identity thereto, a LC CDR1 comprising SEQ ID No: 288 or a sequence with at least 60%, 70%, 80%, 90% or 95% sequence identity thereto, a LC CDR2 comprising SEQ ID No: 289 or a sequence with at least 60%, 70%, 80%, 90% or 95% sequence identity thereto and a LC CDR3 comprising SEQ ID No: 290 or a sequence with at least 60%, 70%, 80%, 90% or 95% sequence identity thereto;
an antibody comprising a HC CDR1 comprising SEQ ID No: 365 or a sequence with at least 60%, 70%, 80%, 90% or 95% sequence identity thereto, a HC CDR2 comprising SEQ ID No: 366 or a sequence with at least 60%, 70%, 80%, 90% or 95% sequence identity thereto, a HC CDR3 comprising SEQ ID No: 367 or a sequence with at least 60%, 70%, 80%, 90% or 95% sequence identity thereto, a LC CDR1 comprising SEQ ID No: 368 or a sequence with at least 60%, 70%, 80%, 90% or 95% sequence identity thereto, a LC CDR2 comprising SEQ ID No: 369 or a sequence with at least 60%, 70%, 80%, 90% or 95% sequence identity thereto and a LC CDR3 comprising SEQ ID No: 370 or a sequence with at least 60%, 70%, 80%, 90% or 95% sequence identity thereto;
an antibody comprising a HC CDR1 comprising SEQ ID No: 665 or a sequence with at least 60%, 70%, 80%, 90% or 95% sequence identity thereto, a HC CDR2 comprising SEQ ID No: 666 or a sequence with at least 60%, 70%, 80%, 90% or 95% sequence identity thereto, a HC CDR3 comprising SEQ ID No: 667 or a sequence with at least 60%, 70%, 80%, 90% or 95% sequence identity thereto, a LC CDR1 comprising SEQ ID No: 668 or a sequence with at least 60%, 70%, 80%, 90% or 95% sequence identity thereto, a LC CDR2 comprising SEQ ID No: 669 or a sequence with at least 60%, 70%, 80%, 90% or 95% sequence identity thereto and a LC CDR3 comprising SEQ ID No: 670 or a sequence with at least 60%, 70%, 80%, 90% or 95% sequence identity thereto;
an antibody comprising a HC CDR1 comprising SEQ ID No: 751 or a sequence with at least 60%, 70%, 80%, 90% or 95% sequence identity thereto, a HC CDR2 comprising SEQ ID No: 752 or a sequence with at least 60%, 70%, 80%, 90% or 95% sequence identity thereto, a HC CDR3 comprising SEQ ID No: 753 or a sequence with at least 60%, 70%, 80%, 90% or 95% sequence identity thereto, a LC CDR1 comprising SEQ ID No: 754 or a sequence with at least 60%, 70%, 80%, 90% or 95% sequence identity thereto, a LC CDR2 comprising SEQ ID No: 755 or a sequence with at least 60%, 70%, 80%, 90% or 95% sequence identity thereto and a LC CDR3 comprising SEQ ID No: 756 or a sequence with at least 60%, 70%, 80%, 90% or 95% sequence identity thereto or
an antibody comprising a HC CDR1 comprising SEQ ID No: 761 or a sequence with at least 60%, 70%, 80%, 90% or 95% sequence identity thereto, a HC CDR2 comprising SEQ ID No: 762 or a sequence with at least 60%, 70%, 80%, 90% or 95% sequence identity thereto, a HC CDR3 comprising SEQ ID No: 763 or a sequence with at least 60%, 70%, 80%, 90% or 95% sequence identity thereto, a LC CDR1 comprising SEQ ID No: 764 or a sequence with at least 60%, 70%, 80%, 90% or 95% sequence identity thereto, a LC CDR2 comprising SEQ ID No: 765 or a sequence with at least 60%, 70%, 80%, 90% or 95% sequence identity thereto and a LC CDR3 comprising SEQ ID No: 766 or a sequence with at least 60%, 70%, 80%, 90% or 95% sequence identity thereto.

The companion animal may be a dog or a cat.

In one embodiment, the antibody or fragment binds to canine OX40L.

In one embodiment, the antibody or fragment is capable of
a) Reducing, inhibiting or neutralising OX40 activity or activation in the companion animal or in a cell of the companion animal;
b) Modifying secretion of a cytokine in the companion animal or in a cell of the companion animal and/or
c) Decreasing proliferation of leukocytes in the companion animal or in a cell of the companion animal.

In one embodiment, the antibody or fragment is capable of
a) Reducing, inhibiting or neutralising OX40 activity or activation in the companion animal or in a cell of the companion animal;
b) Decreasing secretion of inflammatory cytokine in the companion animal or in a cell of the companion animal and/or
c) Decreasing secretion of an inflammatory chemokine or chemokine receptor in the companion animal or in a cell of the companion animal and/or
d) Increasing the secretion of suppressive cytokine(s) in the companion animal or in a cell of the companion animal and/or
e) Increasing the secretion of suppressive chemokines(s) or chemokine receptors in the companion animal or in a cell of the companion animal and/or
f) Decreasing proliferation of leukocytes in the companion animal.

Suitable assays assessing these properties, such as a Mixed Lymphocyte Reaction (MLR) assay or HEK-blue assay for measuring an inhibition of NFkB activity, are described herein, such as the assays shown in the examples, e.g. the PBMC activation assay. Other assays are known to the skilled person and may also be used.

The cytokine or cytokine receptor may be selected from TNF alpha, IL-1 Ra, IL-2, IL-3, IL-4, IL-5, IL-6, IL-8, IL-9, IL-10, IL-13, IL-17, RANTES, GM-CSF, TGF-β and interferon gamma.

In one embodiment, the antibody or fragment binds to canine OX40. The antibody or fragment may be capable of reducing, inhibiting or neutralising OX40 activity or activation in the companion animal or in a cell of the companion animal.

In one embodiment of the forgoing and the various aspects of the invention relating to antibodies that bind OX40 or OX40L, the antibody or fragment is a fully canine, chimeric or caninized antibody. The terms fully canine and canine are used interchangeably herein. According to a preferred embodiment, the antibody is canine (i.e. fully canine).

For example, said fragment is selected from a F(ab′)2, Fab, Fv, scFv, heavy chain, light chain, variable heavy (VH), variable light (VL) chain, CDR region, single VH or VL domain, maxibodies, minibodies, intrabodies, diabodies, triabodies, tetrabodies, and bis-scFv, and polypeptides that contain at least a portion of an immunoglobulin that is sufficient to confer specific antigen binding to the polypeptide.

In one embodiment, the antibody or fragment is conjugated to another moiety. The antibody or fragment may comprise a therapeutic moiety, half life extending moiety or label.

In another aspect, the invention relates to a binding molecule comprising an antibody or fragment as described above.

In another aspect, the invention relates to an antibody or fragment or the binding molecule as described above for use in the treatment of a disease.

In another aspect, the invention relates to a pharmaceutical composition comprising an antibody or fragment thereof or binding molecule as described above.

In another aspect, the invention relates to an antibody or fragment thereof, binding molecule pharmaceutical as described above for use in the treatment of an OX40 or OX40L-mediated disease.

In another aspect, the invention relates to methods of treating or preventing an OX40 or OX40L-mediated disease comprising administering to a subject in need thereof an antibody or fragment, binding molecule or the pharmaceutical composition as described above.

For example, the disease is selected from an inflammatory or autoimmune disease.

The disease may be an inflammatory skin diseases, including atopic dermatitis, allergic dermatitis, pruritus, psoriasis, scleroderma, or eczema; responses associated with inflammatory bowel disease (such as Crohn's disease and ulcerative colitis); ischemic reperfusion; adult respiratory distress syndrome; asthma; meningitis; encephalitis; uveitis; autoimmune diseases such as rheumatoid arthritis, Sjorgen's syndrome, vasculitis; diseases involving leukocyte diapedesis; central nervous system (CNS) inflammatory disorder, multiple organ injury syndrome secondary to septicaemia or trauma, bacterial pneumonia, antigen-antibody complex mediated diseases; inflammations of the lung, including pleurisy, alveolitis, vasculitis, pneumonia, chronic bronchitis, bronchiectasis, and cystic fibrosis.

In one embodiment, the antibody or fragment, binding molecule, pharmaceutical composition is administered together with one or more therapeutic agent.

For example, said one or more therapeutic agent is selected from rapamycin (sirolimus), tacrolimus, cyclosporine (e.g. Atopica®), corticosteroids (e.g. methylprednisolone), methotrexate, mycophenolate mofetil, anti-CD28 antibodies, anti-IL12/IL-23 antibodies, anti-CD20 antibodies, anti-CD30 antibodies, CTLA4-Fc molecules, CCR5 receptor antagonists, anti-CD40L antibodies, anti-VI_A4 antibodies, anti-LFA1 antibodies, fludarabine, anti-CD52 antibodies, anti-CD45 antibodies, cyclophosphamide, anti-thymocyte globulins, anti-complement C5 antibodies, anti-a4b7 integrin antibodies, anti-IL6 antibodies, anti-IL6-R antibodies, anti-IL2R antibodies, anti-CD25 antibodies, anti-TNFa (TNFα)/TNFa-Fc molecules, HDAC inhibitors, JAK inhibitors, such as JAK-1 and JAK-3 inhibitors, anti-IL-31 antibodies, SYK inhibitors, anti-IL-4Ra antibodies, anti-IL-13 antibodies, anti-TSLP antibodies, PDE4 inhibitors, lokietmab (Cytopoint®), and oclacitinib (Apoquel®).

In another aspect, the invention relates to a method of decreasing the secretion of cytokines comprising administering to a subject in need thereof an antibody or fragment, binding molecule or a pharmaceutical composition as described above.

In another aspect, the invention relates to a multispecific binding agent comprising an antibody or fragment or a binding molecule as described above.

In another aspect, the invention relates to a combination therapy comprising antibody or fragment, binding molecule or pharmaceutical composition as described above.

In another aspect, the invention relates to an immunoconjugate comprising an antibody or fragment or binding molecule as described above.

In another aspect, the invention relates to a kit comprising an antibody or fragment thereof, binding or pharmaceutical composition as described above.

In another aspect, the invention relates to vector comprising a nucleic acid as above.

In another aspect, the invention relates to host cell comprising a nucleic acid or a vector as described above where the host cell is optionally selected from a mammalian, yeast, plant or bacterial cell.

In another aspect, the invention relates to a method for detecting OX40L or OX40 in a companion animal comprising contacting a test sample with an antibody or fragment or a binding molecule as described above.

In another aspect, the invention relates to a trimeric soluble companion animal OX40L extra cellular domain probe and its use in in a method of screening for companion animal OX40L antibodies.

The invention is described in the following non-limiting figures and tables.

### FIGURES

FIG. 1. Canine OX40 and OX40L gene and protein structures. A. The predicted number and arrangement of exons within the dog genome. Exons are represented by boxes. Numbers above the line represent the predicted size of each exon in nucleotides. Numbers below the line represent the predicted size of introns in nucleotides, 5′ UTR and 3′ UTR (shaded boxes). Predicted gene structures were made based on NCBI Assembly ID: 317138 (CanFam3.1). The size and sequence of the predicted transcripts was confirmed by RT-PCR using RNA extracted from activated PBMCs. B. Cartoon representations of OX40 and OX40L proteins.

FIG. 2. Alignment of the canine OX40 long and short amino acids sequences (SEQ ID Nos. 4 and 6). Based on protein sequence homology with OX40 from other species, exon 6 is predicted to contain the transmembrane domain. Cartoon representations of these sequences are shown in FIG. 1B.

FIG. 3. Relative abundance of the two canine OX40 splice variants in PHA-activated canine PBMC's, as determined by the number of E. coli colonies transformed with total OX40 cDNA. The long and short splice variants were identified from individual colonies by diagnostic PCR. Measurements were taken on independent PBMC samples, 1 and 4 days after activation. Insert: PCR discrimination of short (S) and long (L) splice variants. +ve=positive control. 1 kb Plus DNA Ladder (NEB), with brighter bands at 0.5, 1.0 and 3.0 bp.

FIG. 4. Serum antibody titres from immunised and non-immunised mice, measured using flow cytometry. A. In non-immunised mice there was no appreciable antibody binding to untransfected cells (Δ) or those stably expressing OX40L (◯). B. Serum from immunised mice was collected 10-days after the first boost and yielded a clear distinction between untransfected/wild-type cells (∇) and those stably expressing OX40L (□). In this example, hydrodynamic tail vein injection of OX40L expression plasmid was used for the prime immunisation and OX40L expressing mouse embryonic fibroblasts was used for subsequent boosts.

FIG. 5. Schematic representation of soluble proteins containing canine OX40L extra-cellular domain (ECD) (Seq ID Nos 807-811). A. Monomeric canine OX40L probe containing monomeric human IgG1 (mvhfc), 6×histidine (HIS) tag and tobacco etch virus (TEV) protease cleaving peptide. B. Trimeric canine OX40L probes containing chicken tenascin C trimerization domain and either human IgG1 Fc or HIS tags. The term trimeric refers to the conformation of OX40L extra cellular domain.

FIG. 6. Single-dose cell based binding assay of canine OX40L antibodies. Histograms show the overlayed intensity of signal obtained by flow cytometry of OX40L expressing HEK293 cells or the parental line stained with candidate OX40L antibodies and subsequently with a fluorescently labelled secondary antibody. All antibodies shown in the figure bind OX40L-expressing HEK cells with higher affinity compared to the parental line, with exception with the one labelled as non-binder. Geo mean intensity data are presented in Table 4.

FIG. 7. Functional assay to measure OX40-OX40L interactions. A. Representative curves showing the enzymatic activity of secreted alkaline phosphatase, that was liberated 6 hrs, 24 hrs and 48 hrs after mixing HEK-Blue-OX40 with HEK-OX40L cells. All curves are baseline subtracted with the signal obtained with culture media alone.

FIG. 8. Single-dose cell based blocking assay based on HEK blue system to determine the blocking effect of candidate antibodies on OX40-OX40L interaction. Data are normalised with a maximum signal (100%) defined by the response obtained in the absence of antibodies and a minimum signal (0%) measured in the presence of HEK-blue-OX40 cells alone.

FIG. 9. HPLC-SEC chromatogram for antibodies PMX050, 51, 53 and 63. Quantification of the percentage of monomeric for is presented in Table 9.

FIG. 10. Candidate antibody biophysical stability measurement using Uncle (Unchained Labs) for antibodies PMX051 and 063. A. Data shows the intensity and mass distribution (left and centre graphs, respectively) used to calculate the initial diameter of the molecules, and B. SLS/DLS data used to calculate Tm₁ and Tagg₂₆₆. Data are presented in Table 9.

FIG. 11. Quantification of the first melting temperature (Tm1) and aggregation temperatures as measured by fluorescence 266 nm and 473 nm of PMX051, PMX063 PMX 097, PMX098 and PMX154 molecules after incubation at 37° C. over a period of 28 days. Data show that the tendency of all 5 molecules to melt or aggregate are relatively stable when incubated at 37° C. for 28 days. Data were acquired using an UNCLE machine (UNChained Labs).

FIG. 12. Quantification of the average molecule size of PMX051, PMX063 PMX 097, PMX098 and PMX154 molecules after incubation at 37° C. over a period of 28 days. Data show the different propensity of the 5 molecules to aggregate overtime at 37° C. PMX097 and PMX154 demonstrated the best stability when incubated at 37° C. for 28 days. Data were acquired using an UNCLE machine (UNChained Labs).

FIG. 13. Pharmacokinetic profile of PMX097 (triangles) and PMX154 (squares) in dog serum after intravenous injection of 3.0 mg of antibody per kg of body weight. Data were analysed by fitting a two-phase decay curve using Prism. Degradation phase: PMX097=8.5 days; PMX154=8.9 days.

FIG. 14. Canine interferon gamma (IFNY) ELISpot performed on PBMC isolated from dogs treated with PMX097 or PMX154 at the indicated dose before KLH immunisation accordingly to the protocol exemplified in table 11. The data show the number of spots obtained after culturing PBMC with either no stimulation (A.) or in presence of 50 μg/ml KLH (B-C.). Both PMX097 and PMX154 reduced T cell activation for up to 35 post single dosing when administered at 0.5 mg/kg and for up to 77 days post single dosing when administered at 3.0 mg/kg.

FIG. 15. Anti-KLH IgM (A, B) and IgG (C, D) serum titre performed on serum from dogs treated with PMX097 or PMX154 at the indicated dose before KLH immunisation accordingly to the protocol exemplified in table 11. Data show the results obtained from serum isolated from dogs at different time points and a detailed view on days 21 and 98 post dosing. Both PMX097 and PMX154 reduced anti-KLH IgM and IgG serum titre at both 3.0 mg/kg and 0.5 mg/kg for up to 77 days post single dosing.

FIG. 16. Haematoxylin and eosin (HE) staining on sections from skin biopsies collected from dogs treated with PMX097 or PMX154 2 days after intradermal KLH injection. The images show the subcutaneous fat area. (A, B) Biopsies from vehicle control injected dogs collected at the KLH injection site (B) or a non-injected skin area (A). (C, D) Biopsies collected at the KLH injection site from dogs treated with PMX154 at 3.0 mg/kg (C) or 0.5 mg/kg (D). (E, F) Biopsies collected at the KLH injection site from dogs treated with PMX097 at 3.0 mg/kg (E) or 0.5 mg/kg (F). Both PMX097 and PMX154 reduce the presence of immune infiltrates compared to vehicle control dogs. PMX154 nearly completely abrogated the presence of immune infiltrates.

Table 1. Amino Acid Residues and Examples of Conservative Amino Acid Substitutions.

Table 2. Nucleic acid and amino acid sequences.

Table 3. VH and VL gene usage

Table 4. Geomeans obtained from cell binding assay.

Table 5. Normalised cell-based inhibition assay based on HEK blue system, demonstrating the percentage of OX40 signal inhibition of candidate antibodies. Data are normalised with a maximum inhibition (100%) defined as the SEAP activity detected in samples with HEK blue OX40 cells alone and a minimum signal (0%) measured in samples with HEK blue OX40 and HEK OX40L cells, the absence of anti-canine OX40L antibodies.

Table 6: Inhibition of IFN-γ release by candidate OX40L antibodies from activated PBMCs in the presence of OX40L expressing HEK cells. Anti-OX40L antibodies were tested in ten independent experiments. Level of IFN-γ in control samples in each experiment is also indicated. Activated PBMC only=PBMC activated in presence of CD3-CD28 antibodies only and no anti-OX40L antibody. Activated PBMC plus HEK WT=PBMC activated in presence of CD3-CD28 antibodies and WT HEK cells and no anti-OX40L antibody. Activated PBMC plus HEK OX40L=PBMC activated in presence of CD3-CD28 antibodies and HEK cells expressing OX40L and no anti-OX40L antibody.

Table 7: Normalised IFN-γ levels in supernatant from PBMC activated with CD3 and CD28 antibodies in presence of OX40L expressing HEK cells and the indicated anti-OX40L antibody. Data from Table 6 were normalised using GraphPad as such IFN-γ levels in wells with PBMC activated in presence of CD3-CD28 antibodies only and no anti-OX40L antibody was set as 0% and IFN-γ levels in wells with PBMC activated in presence of CD3-CD28 antibodies and HEK cells expressing OX40L and no anti-OX40L antibody was set as 100%.

Table 8: Functional affinity of anti OX40L antibodies for trimeric canine OX40L determined by surface plasmon resonance (SPR). Data were analysed using Biacore Insight Evaluation Software. Curves were fitted using a bivalent analyte binding model.

Table 9: Protein stability determination of OX40L antibodies. Tm₁=melting (unfolding) temperature 1; Tagg₂₆₆=onset aggregation temperature determined by static light scattering at 266 nm; initial diameter=average molecule diameter before temperature increase; % monomer (SEC)=% of monomers determined by HLPLC-SEC.

Table 10: Long term stability of PMX097 and PMX154 when incubated at 4° C. over a period of four months. Data show melting temperature 1 (Tm1), aggregating temperature measured at 266 nm (Tagg 266 nm) the average molecular diameter, (Z AVG diam) and the polydispersity index (PDI).

Table 11: Treatment regime of dogs in the single dose antigen recall dog study.

Table 12: Histological findings in non-injected skin areas from dogs in the single dose antigen recall study.

Table 13: Histological findings in KLH-injected skin areas from dogs in the single dose antigen recall study.

### DETAILED DESCRIPTION

OX40 and its binding partner OX40L are part of the TNF superfamily, and OX40 signalling is a co-stimulatory pathway in promoting T cell activation. OX40 (the receptor) is expressed on the surface of T-cells, and OX40L on both the surface of T-cells and antigen presenting cells such as B cells and macrophages. Neither OX40 nor OX40L are constitutively expressed, but increase 24-72 hours following activation of their respective cells. OX40L binding to OX40 receptors on T-cells promote T cell survival, proliferation and cytokine production. OX40 therefore has a critical role in establishing, maintaining and modulating an immune response.

In humans, both the density of OX40L and the number of OX40 positive cells is significantly greater in the lesional dermis than in the healthy-looking dermis in atopic dermatitis, and blockade of OX40/OX40L signalling modulates several pro-inflammatory responses. OX40L also controls the response of dendritic cells to thymic stromal lymphopoietin (TSLP), which results in IL-21 and CXCL13 production. The OX40/OX40L axis consequently offers the possibility of modulating multiple pro-inflammatory responses, as its co-stimulatory signal sits upstream of several cellular processes that release pro-inflammatory cytokines.

OX40 signalling has been linked to various diseases such as allergy, asthma, and diseases associated with autoimmunity and inflammation, which includes multiple sclerosis, rheumatoid arthritis, inflammatory bowel disease, graft-versus-host disease, experimental autoimmune encephalomyelitis (EAE), experimental leishmaniasis, collagen-induced arthritis, colitis (such as ulcerative colitis), contact hypersensitivity reactions, diabetes, Crohn's Disease, and Grave's Disease. Evidence in humans suggests that disruption of the OX40/OX40L axis reduces proliferative responses, such as immune cells, and could be used to prevent, treat or ameliorate the symptoms of a number of diseases, including atopic dermatitis, diabetes and cancer. Antibodies to either OX40 or OX40L could be used to achieve this disruption.

However, the role of antibody treatments for OX40/OX40L mediated diseases in companion animals, such as atopic dermatitis, e.g. in dogs, has not been investigated previously.

Thus, in a first aspect, the invention relates to an antibody or fragment thereof that specifically binds to companion animal OX40L or to companion animal OX40.

Companion animals of the invention are suitably selected from dogs, cats, horses, birds, rabbits, goats, reptiles, fish and amphibians. A dog is a preferred companion animal of the invention. A cat is a preferred companion animal of the invention. A horse is a preferred companion animal of the invention.

For the avoidance of doubt, a human is not a companion animal.

In one aspect, the companion animal is a dog.

In one aspect, the companion animal is a cat.

In one aspect, the companion animal is a horse.

In one embodiment, antibodies and fragments described herein bind specifically to wild type canine OX40L. The amino acid sequence (SEQ ID No.1) and nucleotide sequences for wild type canine OX40L are shown in Table 2 (SEQ ID No. 2). Antibodies and fragments described herein bind specifically to SEQ ID No.1. In one embodiment, antibodies and fragments described herein bind specifically to variants of SEQ ID No.1.

In one embodiment, antibodies and fragments described herein bind specifically to wild type canine OX40. The amino acid sequence (SEQ ID Nos. 4 and 6) and nucleotide sequences for wild type canine OX40 are shown in table 2 (SEQ ID Nos. 3 and 5). As explained in the examples, two different splice variants were identified. Antibodies and fragments described herein bind specifically to proteins codified by SEQ ID No. 3 and/or 5. In one embodiment, antibodies and fragments described herein bind specifically to proteins codified by variants of SEQ ID No. 3 and/or 5 (SEQ ID Nos. 4 and 6).

Variants of the sequences described above may have at least 75%, 80%, 85%, 90% or 95% sequence identity to the sequences shown above.

As used herein, the terms “homology” or “identity” generally refers to the percentage of amino acid residues in a sequence that are identical with the residues of the reference polypeptide with which it is compared, after aligning the sequences and in some embodiments after introducing gaps, if necessary, to achieve the maximum percent homology, and in some embodiments not considering any conservative substitutions as part of the sequence identity. Thus, the percent homology between two amino acid sequences is equivalent to the percent identity between the two sequences. Neither N- or C-terminal extensions, tags or insertions shall be construed as reducing identity or homology. Methods and computer programs for the alignment are well known. The percentage identity between two amino acid sequences can be determined using well known mathematical algorithms. As used herein the term “sequence similarity” generally refers to the percentage of amino acid residues in a sequence that are identical and/or comprise conservative amino acid substitutions, where the substituted amino acid has similar physicochemical properties. For example, where a positively charged amino acid has been replaced by a different positively charged amino acid. Percentage similarity may be calculated using tools such as “EMBOSS Needle” which implements the Needleman-Wunch algorithm for the pairwise percent identities or similarities, amino acids may be considered similar if they have a positive score in the BLOSUM62 matrix. In an embodiment, where the term “sequence identity” is used herein it may be replaced by the term “sequence similarity”.

Unless otherwise specified, the term OX40L as used herein refers to a companion animal OX40L, e.g. dog or cat OX40L. OX40L is also known as “OX40 Antigen Ligand”, “OX40 Ligand”, “CD252”, “TNFSF4” and “CD134 Ligand”. In one embodiment, the antibody or fragment thereof binds to dog OX40L. In one embodiment, the antibody or fragment thereof binds to cat OX40L.

Unless otherwise specified, the term OX40 as used herein refers to a companion animal OX40, e.g. dog or cat OX40. OX40 is also known as “TNFR Superfamily Member 4”, “TNFRSF4”, “OX40 Antigen” and “CD134”. In one embodiment, the antibody or fragment thereof binds to dog OX40L. In one embodiment, the antibody or fragment thereof binds does not bind to cat OX40L. In one embodiment, the antibody or fragment thereof is not 7D6 as disclosed in Willett et al.

The terms “OX40L binding molecule/protein/polypeptide/agent/moiety”, “OX40L antigen binding molecule molecule/protein/polypeptide/agent/moiety”, “anti-OX40L antibody”, “anti-OX40L antibody fragment” all refer to a molecule capable of specifically binding to the companion animal OX40L, e.g. dog or cat OX40L antigen. The binding reaction may be shown by standard methods, for example with reference to a negative control test using an antibody of unrelated specificity.

The terms “OX40 binding molecule/protein/polypeptide/agent/moiety”, “OX40 antigen binding molecule molecule/protein/polypeptide/agent/moiety”, “anti-OX40 antibody”, “anti-OX40 antibody fragment” all refer to a molecule capable of specifically binding to the companion animal OX40, e.g. dog or cat OX40 antigen. The binding reaction may be shown by standard methods, for example with reference to a negative control test using an antibody of unrelated specificity.

In some embodiments, the antibody or fragment provided herein binds to an OX40L epitope that is a three-dimensional surface feature of a OX40L polypeptide. In some embodiments, the antibody or fragment provided herein binds to an epitope comprising a polypeptide from a single subunit, or a conformational epitope arising from a multimeric form, (e.g., an epitope in a monomeric or trimeric form of an OX40L polypeptide). A region of an OX40L polypeptide contributing to an epitope may be contiguous amino acids of the polypeptide or the epitope may come together from two or more non-contiguous regions of the polypeptide. A OX40L epitope may be present in (a) the trimeric form (“a trimeric OX40L epitope”) of OX40L, (b) the monomeric form (“a monomeric OX40L epitope”) of OX40L, (c) both the trimeric and monomeric form of OX40L, (d) the trimeric form, but not the monomeric form of OX40L, or (e) the monomeric form, but not the trimeric form of OX40L

For example, in some embodiments, the epitope is only present or available for binding in the trimeric form, but is not present or available for binding in the monomeric form by an anti-OX40L antibody. In other embodiments, the OX40L epitope is linear feature of the OX40L polypeptide (e.g., in a trimeric form or monomeric form of the OX40L polypeptide). Antibodies provided herein may specifically bind to (a) an epitope of the monomeric form of OX40L, (b) an epitope of the trimeric form of OX40L, (c) an epitope of the monomeric but not the trimeric form of OX40L, (d) an epitope of the trimeric but not the monomeric form of OX40L, or (e) both the monomeric form and the trimeric form of OX40L. In some embodiments, the antibodies provided herein specifically bind to an epitope of the trimeric form of OX40L but do not specifically bind to an epitope the monomeric form of OX40L. In some embodiments, the antibodies provided herein bind to an epitope of the monomeric form of OX40L and may or may not bind to the trimeric form.

An antibody or fragment thereof “which binds” or is “capable of binding” an antigen of interest, e.g. companion animal OX40L or companion animal OX40 respectively, is one that binds the antigen with sufficient affinity such that the antibody or fragment is useful as a therapeutic agent in targeting a cell or tissue expressing the antigen OX40 or OX40L respectively as described herein.

Antibodies and fragments thereof as described herein bind specifically to the target companion animal OX40L or the target companion animal OX40 respectively. For example, in one embodiment, antibodies and fragments thereof as described herein bind specifically to canine OX40L. In another embodiment, antibodies and fragments thereof as described herein bind specifically to feline OX40L. For example, in one embodiment, antibodies and fragments thereof as described herein bind specifically to canine OX40. In another embodiment, antibodies and fragments thereof as described herein bind specifically to feline OX40. The term “specifically” in the context of antibody binding, refers to high avidity and/or high affinity binding of an antibody to a specific antigen, i.e., a polypeptide, or epitope. In many embodiments, the specific antigen is an antigen (or a fragment or subfraction of an antigen) used to immunize the animal host from which the antibody-producing cells were isolated.

In other words, binding to the OX40L or OX40 antigen is stronger than binding of the same antibody to other antigens, i.e. measurably different from a non-specific interaction. Thus, in one embodiment, the antibodies or fragments of the invention do not cross react with mouse or human OX40L or OX40 antigen.

The term “specific binding” or “specifically binds to” or is “specific for” a particular polypeptide or an epitope on a particular polypeptide target as used herein can be exhibited, for example, by a molecule having a KD (K_{d}) for the target of at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or lower. In one embodiment, the KD (K_{d}) is 10⁻⁹ M or lower. In a preferred embodiment the KD (K_{d}) is 10⁻⁹ M or lower, for example 10⁻¹⁰ M. In an embodiment the antibodies of the invention have a KD (K_{d}) for the target of about 10⁻⁶ M to 10⁻¹² M, or about 10⁻⁷ M to 10⁻¹² M, or about 10⁻⁸ M to 10⁻¹² M, or about 10⁻⁹ M to 10⁻¹² M, or about 10⁻¹⁰ M to 10⁻¹² M, or about 10⁻¹¹ M to 10⁻¹² M. In one embodiment, the term “specific binding” refers to binding where a molecule binds to a particular polypeptide or epitope on a particular polypeptide without substantially binding to any other polypeptide or polypeptide epitope.

In one embodiment, antibodies of the invention are antagonistic antibodies that bind specifically to companion animal OX40L, for example canine OX40L.

As used herein, an “antagonist” or “inhibitor” of OX40L/OX40 refers to a ligand (e.g., antibody or fragment) that is capable of inhibiting or otherwise decreasing one or more of the biological activities of OX40L/OX40, such as in a cell expressing OX40L/OX40 or in a cell expressing an OX40L/OX40 ligand. For example, in certain embodiments, antibodies of the invention are antagonist antibodies that inhibit or otherwise decrease secretion of a cytokine from a cell having a cell surface-expressed OX40L/OX40 when said antibody is contacted with said cell. In some embodiments, an antagonist of OX40L (e.g., an antagonistic antibody of the invention) may, for example, act by inhibiting or otherwise decreasing the activation and/or cell signalling pathways of the cell expressing OX40L/OX40, thereby inhibiting a OX40L/OX40-mediated biological activity of the cell the relative to the OX40L/OX40-mediated biological activity in the absence of antagonist. In certain embodiments, the antibodies provided herein are fully canine, antagonistic anti-OX40L/OX40 antibodies, preferably fully canine, monoclonal, antagonistic anti-OX40L/OX40 antibodies.

The term “antibody” as used herein broadly refers to any immunoglobulin (Ig) molecule, or antigen binding portion thereof, comprised of four polypeptide chains, two heavy (H) chains and two light (L) chains, or any functional fragment, mutant, variant, or derivation thereof, which retains the essential epitope binding features of an Ig molecule.

In a full-length antibody, each heavy chain is comprised of a heavy chain variable region or domain (abbreviated herein as HCVR) and a heavy chain constant region. The heavy chain constant region is comprised of three domains, C_{H}1, C_{H}2 and C_{H}3. Each light chain is comprised of a light chain variable region or domain (abbreviated herein as LCVR) and a light chain constant region. The light chain constant region is comprised of one domain, C_{L}.

The heavy chain and light chain variable regions can be further subdivided into regions of hypervariability, termed complementarity determining regions (CDR), interspersed with regions that are more conserved, termed framework regions (FR). Each heavy chain and light chain variable region is composed of three CDRs and four FRs, arranged from amino-terminus to carboxy-terminus in the following order: FR1, CDR1, FR2, CDR2, FR3, CDR3, FR4.

Immunoglobulin molecules can be of any type, class or subclass (e.g., for dogs IgG, IgE, IgM, IgD, IgA and IgY; e.g. canine IgG subtype, for example IgG-A, IgG-B, IgG-C, and IgG-D). In canine, there are four IgG heavy chains referred to as A, B, C, and D. These heavy chains represent four different subclasses of dog IgG, which are referred to as IgGA, IgGB, IgGC and IgGD. The DNA and amino acid sequences of these four heavy chains were first identified by Tang et al. (Vet. Immunol. Immunopathol. 80: 259-270 (2001)). Exemplary amino acid and DNA sequences for these heavy chains are also available from the GenBank data bases (IgGA: accession number AAL35301.1, IgGB: accession number AAL35302.1, IgGC: accession number AAL35303.1, IgGD: accession number AAL35304.1). Canine antibodies also contain two types of light chains, kappa and lambda (GenBank accession number kappa light chain amino acid sequence ABY 57289.1, GenBank accession number ABY 55569.1). Amino acid sequences for IgG-A, IgG-B, IgG-C and IgG-D as used by the inventors and according to the aspects and embodiments of the invention are shown in Table 2.

The term “CDR” refers to the complementarity-determining region within antibody variable sequences. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs can be defined differently according to different systems known in the art.

The Kabat Complementarity Determining Regions (CDRs) are based on sequence variability and are the most commonly used (Kabat et al., (1971) Ann. NY Acad. Sci. 190:382-391 and Kabat, et al., (1991) Sequences of Proteins of Immunological Interest, Fifth Edition, U.S. Department of Health and Human Services, NIH Publication No. 91-3242). Chothia refers instead to the location of the structural loops (Chothia and Lesk J. Mol. Biol. 196:901-917 (1987)). The Kabat numbering system is generally used when referring to a residue in the variable domain (approximately residues 1-107 of the light chain and residues 1-113 of the heavy chain). Another system is the ImMunoGeneTics (IMGT) numbering scheme. The IMGT numbering scheme is described in Lefranc et al., Dev. Comp. Immunol., 29, 185-203 (2005). These terms, which are recognized in the art, refer to a system of numbering amino acid residues which are more variable (i.e., hypervariable) than other amino acid residues in the heavy and light chain variable regions of an antibody, or an antigen binding portion.

The IMGT numbering scheme is used herein unless otherwise specified.

The antibody to OX40L or OX40 according to the invention may be a canine, humanized, feline, chimeric antibody, felinized or caninized antibody.

A “chimeric antibody” is a recombinant protein that contains the variable domains including the complementarity determining regions (CDRs) of an antibody derived from one species, while the constant domains of the antibody molecule are derived from those of another species, e.g., a canine antibody. An exemplary chimeric antibody is a chimeric human-canine antibody.

A “humanized antibody” is a recombinant protein in which the CDRs from an antibody from one species; e.g., a rodent, canine, feline antibody, are transferred from the heavy and light variable chains of the rodent, canine or feline antibody into human heavy and light variable domains (e.g., framework region sequences). The constant domains of the antibody molecule are derived from those of a human antibody. In certain embodiments, a limited number of framework region amino acid residues from the parent (e.g., rodent, canine or feline) antibody may be substituted into the human antibody framework region sequences. In an embodiment the CDRs disclosed herein may be transferred from the heavy and light variable chains of the antibody disclosed herein into the heavy and light variable domains (e.g., framework region sequences) of a different species.

As used herein, the term “caninized antibody” refers to forms of recombinant antibodies that contain sequences from both canine and non-canine (e.g., murine) antibodies. In general, the caninized antibody will comprise substantially all of at least one or more typically, two variable domains in which all or substantially all of the hypervariable loops correspond to those of a non-canine immunoglobulin, and all or substantially all of the framework (FR) regions (and typically all or substantially all of the remaining frame) are those of a canine immunoglobulin sequence. A caninized antibody may comprise both the three heavy chain CDRs and the three light chain CDRS from a murine or human antibody together with a canine frame or a modified canine frame. A modified canine frame comprises one or more amino acids changes that can further optimize the effectiveness of the caninized antibody, e.g., to increase its binding to its target. The non-canine sequences, e.g., of the hypervariable loops, may further be compared to canine sequences and as many residues changed to be as similar to authentic canine sequences as possible.

A “speciated” antibody (e.g. humanized, caninized, chimeric, felinized) is one which has been engineered to render it similar to antibodies of the target species. In one embodiment, a “speciated” antibody is greater than about 80%, 85% or 90% similar to antibodies of the target species.

In one embodiment, the antibody or antibody fragment is canine. By canine is meant fully canine. The terms fully canine and canine are used interchangeably herein.

In contrast to speciated antibodies, fully canine antibodies of the present invention have canine variable regions and do not include full or partial CDRs or FRs from another species. Advantageously, fully canine antibodies as described herein have been obtained from transgenic mice comprising canine immunoglobulin sequences. Antibodies produced in these immunised mice are developed through in vivo B cell signalling and development to allow for natural affinity maturation including in vivo V(D)J recombination, in vivo junctional diversification, in vivo pairing of heavy and light chains and in vivo hypermutation. Fully canine antibodies produced in this way generate antibodies with optimal properties for developability, minimizing lengthy lead optimization prior to production at scale. Advantageously, such fully canine antibodies present the lowest possible risk of immunogenicity when introduced into a patient animal which, in turn, facilitates a repeated dosing regimen. Adverse in vivo immunogenicity can be assessed, for example, by assays to identify the production of anti-drug antibodies (ADA), or a loss of efficacy over time in vivo. Given that ex vivo mAb engineering runs the risk of introducing development liabilities, immunogenicity, and reduced affinity (as outlined above), fully canine antibodies of the present invention are, therefore, most likely to be efficacious therapies in a clinical context.

As used herein, the term “felinized antibody” refers to forms of recombinant antibodies that contain sequences from both feline and non-feline (e.g., murine) antibodies. In general, the felinized antibody will comprise substantially all of at least one or more typically, two variable domains in which all or substantially all of the hypervariable loops correspond to those of a non-feline immunoglobulin, and all or substantially all of the framework (FR) regions (and typically all or substantially all of the remaining frame) are those of a feline immunoglobulin sequence. A felinized antibody may comprise both the three heavy chain CDRs and the three light chain CDRs from a murine or human antibody together with a feline frame or a modified feline frame. A modified feline frame comprises one or more amino acids changes that can further optimize the effectiveness of the felinized antibody, e.g., to increase its binding to its target. The non-feline sequences, e.g., of the hypervariable loops, may further be compared to feline sequences and as many residues changed to be as similar to authentic feline sequences as possible.

In one embodiment, the antibody or antibody fragment is feline. By feline is meant fully feline. The terms fully feline and feline are used interchangeably herein.

In contrast to speciated antibodies, fully feline antibodies of the present invention have feline variable regions and do not include full or partial CDRs or FRs from another species. Fully feline antibodies may be obtained from transgenic mice comprising feline immunoglobulin sequences. Antibodies produced in these immunised mice are developed through in vivo B cell signalling and development to allow for natural affinity maturation including in vivo V(D)J recombination, in vivo junctional diversification, in vivo pairing of heavy and light chains and in vivo hypermutation. Fully feline antibodies produced in this way generate antibodies with optimal properties for developability, minimizing lengthy lead optimization prior to production at scale. Advantageously, such fully feline antibodies present the lowest possible risk of immunogenicity when introduced into a patient animal which, in turn, facilitates a repeated dosing regimen. Adverse in vivo immunogenicity can be assessed, for example, by assays to identify the production of anti-drug antibodies (ADA), or a loss of efficacy over time in vivo. Ex vivo mAb engineering runs the risk of introducing development liabilities, immunogenicity, and reduced affinity (as outlined above), as such fully feline antibodies are, therefore, most likely to be efficacious therapies in a clinical context.

As used herein, the term “equinized antibody” refers to forms of recombinant antibodies that contain sequences from both equine and non-equine (e.g., murine) antibodies. In general, the equinized antibody will comprise substantially all of at least one or more typically, two variable domains in which all or substantially all of the hypervariable loops correspond to those of a non-equine immunoglobulin, and all or substantially all of the framework (FR) regions (and typically all or substantially all of the remaining frame) are those of an equine immunoglobulin sequence. An equinized antibody may comprise both the three heavy chain CDRs and the three light chain CDRS from a murine or human antibody together with a feline frame or a modified equine frame. A modified equine frame comprises one or more amino acids changes that can further optimize the effectiveness of the equinized antibody, e.g., to increase its binding to its target. The non-equine sequences, e.g., of the hypervariable loops, may further be compared to equine sequences and as many residues changed to be as similar to authentic equine sequences as possible.

In one embodiment, the antibody or antibody fragment is equine. By equine is meant fully equine. The terms fully equine and equine are used interchangeably herein.

The term “monoclonal antibody” as used herein refers to an antibody derived from a single B or plasma cell. All antibody molecules in a monoclonal antibody preparation are identical except for possible naturally occurring post-translation modifications (e.g., isomerizations, amidations, carbohydrate addition) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen.

The term “epitope” or “antigenic determinant” refers to a site on the surface of an antigen (to which an immunoglobulin, antibody or antibody fragment, specifically binds. Generally, an antigen has several or many different epitopes and reacts with many different antibodies. The term specifically includes linear epitopes and conformational epitopes. Epitopes within protein antigens can be formed both from contiguous amino acids (usually a linear epitope) or non-contiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody or antibody fragment (i.e., epitope mapping) are well known in the art and include, for example, immunoblotting and immunoprecipitation assays, wherein overlapping or contiguous peptides from are tested for reactivity with a given antibody or antibody fragment.

An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in different formats, using either labelled antigen or labelled antibody. The epitope may or may not be a three-dimensional surface feature of the antigen. In certain embodiments, an OX40L epitope is a three-dimensional surface feature of a OX40L polypeptide (e.g., in a trimeric form of a OX40L polypeptide). In other embodiments, a OX40L epitope is linear feature of a OX40L polypeptide (e.g., in a trimeric form or monomeric form of the OX40L polypeptide). Antibodies provided herein may specifically bind to an epitope of the monomeric form of OX40L, an epitope of the trimeric form of OX40L, or both the monomeric form and the trimeric form of OX40L. In specific embodiments, the antibodies provided herein specifically bind to an epitope of the trimeric form of OX40L but do not specifically bind the monomeric form of OX40L. In some embodiments, an antibody may, for example, bind to a monomer/single subunit and block formation of an active trimeric form. Suitably the antibodies bind to the extracellular domain of OX40L.

The term “antigen binding site” refers to the part of the antibody or antibody fragment that comprises the area that specifically binds to an antigen. An antigen binding site may be provided by one or more antibody variable domains. An antigen binding site is typically comprised within the associated V_{H} and V_{L} of an antibody or antibody fragment.

The term antibody as used herein also includes antibody fragments. Specifically, the invention also extends to antibody fragments. An antibody fragment is a portion of an antibody, for example a F(ab′)2, Fab, Fv, scFv, heavy chain, light chain, variable heavy (V_{H}), variable light (V_{L}) chain, CDR region, single V_{H} or V_{L} domain, maxibodies, minibodies, intrabodies, diabodies, triabodies, tetrabodies, and bis-scFv, and polypeptides that contain at least a portion of an immunoglobulin that is sufficient to confer specific antigen binding to the polypeptide. Therefore, an antibody fragment comprises an antigen binding portion.

Antibody fragments are functional fragments of a full-length antibody, that is they retain the target specificity of a full antibody. Recombinant functional antibody fragments, such as Fab (Fragment, antibody), scFv (single chain variable chain fragments) and single domain antibodies (dAbs) have therefore been used to develop therapeutics as an alternative to therapeutics based on mAbs.

An “Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site. “Single-chain Fv” also abbreviated as “sFv” or “scFv” are antibody fragments that comprise the VH and VL antibody domains connected into a single polypeptide chain.

scFv fragments (˜25 kDa) consist of the two variable domains, V_{H} and V_{L}. Naturally, V_{H} and V_{L} domain are non-covalently associated via hydrophobic interaction and tend to dissociate. However, stable fragments can be engineered by linking the domains with a hydrophilic flexible linker to create a single chain Fv (scFv).

The smallest antigen binding fragment is the single variable fragment, namely the variable heavy (V_{H}) or variable light (V_{L}) chain domain. V_{H} and V_{L} domains respectively are capable of binding to an antigen. Binding to a light chain/heavy chain partner respectively or indeed the presence of other parts of the full antibody is not required for target binding. The antigen-binding entity of an antibody, reduced in size to one single domain (corresponding to the V_{H} or V_{L} domain), is generally referred to as a “single domain antibody” or “immunoglobulin single variable domain”. A single domain antibody (˜12 to 15 kDa) has thus either the V_{H} or V_{L} domain.

Thus, in one embodiment, the fragment is selected from a F(ab′)2, Fab, Fv, scFv, heavy chain, light chain, variable heavy (V_{H}), variable light (V_{L}) chain, CDR region, single V_{H} or V_{L} domain, maxibodies, minibodies, intrabodies, diabodies, triabodies, tetrabodies, and bis-scFv, and polypeptides that contain at least a portion of an immunoglobulin that is sufficient to confer specific antigen binding to the polypeptide.

In one embodiment, the invention does not relate to an immunoglobulin domain, e.g. an Fc domain, fused to a companion animal, e.g. canine, OX40L extracellular domain polypeptide fragment or biological equivalent thereof.

The antibodies and antibody fragments of the invention are isolated. The term “isolated” as used herein refers to a moiety that is isolated from its natural environment. For example, the term “isolated” refers to a single domain antibody that is substantially free of other single domain antibodies, antibodies or antibody fragments. Moreover, an isolated single domain antibody may be substantially free of other cellular material and/or chemicals.

In one aspect, the invention relates to an antibody or fragment thereof that binds specifically to companion animal, such as canine, OX40L wherein said antibody blocks binding of OX40L to OX40 and/or inhibits one or more functions associated with binding of OX40L to OX40. Suitably the antibody reduces, inhibits or neutralises OX40 activity in the companion animal. In one embodiment, the antibody or fragment thereof exhibits one or more of the following properties:
a) Is capable of modifying secretion of a cytokine in the cell or animal, and/or
b) Is capable of decreasing proliferation of leukocytes in the companion animal, such as dog.

“Modifying” refers to increasing or decreasing the amount of a compound in the presence of an antibody compared to a control. “Decreasing” or “decreases” as used herein refers to a reduction and the decrease may be at least 10%, 20%, 30%, 40%, 50%, 60%, 70%, 80%, 90%, 95% or more.

For example, the antibody or fragment is capable of
a) Decreasing secretion of inflammatory cytokine in the companion animal or in a cell of the companion animal and/or
b) Decreasing secretion of an inflammatory chemokine or chemokine receptor in the companion animal or in a cell of the companion animal and/or
c) Increasing the secretion of suppressive cytokine(s) in the companion animal or in a cell of the companion animal and/or
d) Increasing the secretion of suppressive chemokines(s) or chemokine receptors in the companion animal or in a cell of the companion animal and/or
e) Decreasing proliferation of leukocytes in the companion animal.

The cytokine may be selected from TNF alpha, IL-2, IL-3, IL-4, IL-5, IL-6, IL-8, IL-9, IL-10, IL-13, IL-17, RANTES, GM-CSF, TGF-β and interferon gamma (IFN-γ). In an embodiment the antibody acts to decrease the secretion of the inflammatory cytokine in the companion animal or in a cell of the companion animal compared to where no antibody is present. In a preferred embodiment the inflammatory cytokine is IFN-γ.

The antibody may act to decrease the secretion of the inflammatory cytokine in the companion animal or in a cell of the companion animal by more than 30%, more than 40%, more than 50%, more than 60%, more than 70%, more than 80%, more than 90% compared to where no antibody is present. The antibody may act to decrease the secretion of the inflammatory cytokine in the companion animal or in a cell of the companion animal by 30% to 100%, 40% to 100%, 50% to 100%, 60% to 100%, 70% to 100%, 80% to 100%, 90% to 100% compared to where no antibody is present. The decrease in secretion of the inflammatory cytokine may be determined using a cell signalling inhibition assay such as that described herein in Example 7 i.e., aPBMC activation assay.

An “inflammatory” compound is one that is involved in promoting inflammation, whereas a “suppressive” compound is one that is involved in suppressing or regulating inflammation. Inflammatory cytokines include interleukin-1 (IL-1), IL-12, and IL-18, TNF alpha, interferon gamma (IFNγ) and GM-CSF. Suppressive or anti-inflammatory cytokines or receptors include IL-4, IL-10, IL-11, IL-13 and TGF-β. The cytokine may be a chemokine. In one embodiment, the chemokine may be selected from CXCL13, CXCR5, for example. The antibody in accordance with the invention may also modify cytokine or chemokine receptor expression.

Assays may be carried out in vitro (e.g. using a cell, cells or tissue) or in vivo.

In one embodiment, binding of OX40L to OX40 in the presence of an antibody in accordance with the invention may be determined in an SPR (surface plasmon resonance) assay, for example. Other methods for determining inhibition of an OX40L/OX40 interaction are described, for example in WO2016/139482 or WO2013/008171 and include, for example, flow cytometry monitoring of antibody binding to recombinant OX40L-expressing cells.

In one embodiment, the ability of an antibody to OX40L to block binding of OX40L to OX40 can be measured by measuring an inhibition of NFkB activity. Suitable assays for measuring NFkB activity include the HEK-blue assay described herein. Accordingly, in one embodiment, the antibody or fragment thereof that binds specifically to companion animal, such as canine, OX40L reduces, inhibits or neutralises OX40R-mediated NFkB activity in a cell-based assay.

In one embodiment, the assay is a heterologous assay in which a companion animal (e.g. dog) OX40 is used in a cell line derived from a different species, e.g. a human cell line such as HEK, substantially as described in Example 7 herein.

In one embodiment, the ability of an antibody to OX40L to block binding of OX40L to OX40 can be measured by measuring a decreased secretion of a cytokine in a cell compared to that observed in the absence of the antibody. In one embodiment, the ability of an antibody to OX40L to block binding of OX40L to OX40 can be measured by measuring an inhibition of IL-2 or INF-gamma secretion from PBMCs. Accordingly, in one embodiment, the antibody or fragment thereof that binds specifically to companion animal, such as canine, OX40L reduces, inhibits or neutralises OX40R-mediated IL-2 or INF-gamma (INFγ) secretion from PBMCs. In another embodiment, the ability of an antibody to OX40L to block binding of OX40L to OX40 can be measured by measuring an inhibition of IL-13 secretion from PBMCs. It will be understood that the ability of an antibody to OX40 to block binding of OX40L to OX40 can be measured in a similar manner.

The antibody or fragment is capable of effecting a decrease of the proliferation of leukocytes (e.g., mononuclear cells) in an in vitro assay wherein the antibody or fragment antagonises OX40L/OX40L receptor interaction.

As is known in the art, the term “leukocytes” includes, for example, one or more of lymphocytes, polymorphonuclear leukocyte and monocytes. As is also readily apparent to the skilled person the term “monocytes” includes, for example, peripheral blood mononuclear cells (PBMCs) or monocyte derived cells, e.g., dendritic cells (DCs).

Leukocyte proliferation may be measured, for example in a Mixed Lymphocyte Reaction (MLR) as described herein. The ability of an antibody in accordance with the invention to decrease proliferation may be measured by comparison to proliferation in the absence of the antibody.

The proliferation of leukocytes, e.g., lamina propria lymphocytes (LPLs), can be assessed using tissue biopsy, staining and histology, as will be apparent to the skilled person. Hematoxylin and eosin stain (H&E stain or HE stain) is, for example, commonly used in histology to look for infiltrating lymphocytes a whole range of human tissue and is one of the principal stains in histology. It is the most widely used stain in medical diagnosis and is often the gold standard, and as such can be used to assess proliferation of leukocytes as per the invention. For example, GI tract tissue (e.g., gut tissue) from a companion animal that is suffering from or at risk of a OX40L-mediated disease or condition can be obtained, stained and assessed for the extent of infiltration of LPLs.

Comparison can be made between such tissue from a companion animal that has received an antibody of the invention compared to the extent of infiltration in tissue obtained from the same animal prior to administration of antibody or from another companion animal that has not received treatment and is at risk of or suffering from the disease or condition. For example, the comparison is between companion animal gut tissues taken from the same (or different) companion animals suffering from e.g. IBD. The anti-OX40L antibody binds to OX40L and regulates cytokine and cellular receptor expression resulting in cytokine levels characteristic of non-disease states. The anti-OX40 antibody binds to OX40 and regulates cytokine and cellular receptor expression resulting in cytokine levels characteristic of non-disease states.

Cytokines are indispensable signals of the mucosa-associated immune system for maintaining normal gut homeostasis. An imbalance of their profile in favour of inflammation initiation may lead to disease states, such as that is observed in inflammatory bowel diseases (IBD), e.g., Crohn's disease (CD) and ulcerative colitis (UC). The role of pro-inflammatory cytokines such as IL-Ia, IL-Iβ, IL-2, -6, -8, -12, -17, -23, IFN-gamma, or TNF alpha in IBD is associated with the initiation and progression of UC and CD. CD is often described as a prototype of T-helper (Th) 1-mediated diseases because the primary inflammatory mediators are the Th1 cytokines such as interleukin (IL)-12, interferon (IFN)-y, and tumour necrosis factor (TNF)-α. The cytokine or cytokine receptor may be selected from TNF alpha, IL-2, IL-3, IL-4, IL-5, IL-6, IL-8, IL-9, IL-10, IL-13, IL-17, RANTES, GM-CSF, TGF-β and interferon gamma.

Further information on suitable assays to assess properties of the antibodies is provided in the examples.

The antibodies disclosed herein can be grouped into different groups i.e., families based on the pairs of V genes that have been used to generate the antibodies. Family 1 comprises antibodies generated from heavy chain V-gene IGHV3-5 and light chain V-gene IGLV3-14. Examples of antibodies in Family 1 include antibodies designated PMX025, PMX026, PMX027, PMX089, PMX090, PMX091, PMX092, PMX093, the sequences of these antibodies are set out in Table 2.

Family 2 comprises antibodies generated from heavy chain V-gene IGHV3-5 and light chain V-gene IGLV3-21. Examples of antibodies in Family 2 include antibodies designated PMX028, PMX029, PMX032, PMX033, PMX034, PMX036, PMX038, PMX042, PMX045, PMX064, PMX082, PMX083 the sequences of these antibodies are set out in Table 2.

Family 3 comprises antibodies generated from heavy chain V-gene IGHV3-5 and light chain V-gene IGLV3-24. Examples of antibodies in Family 3 include antibodies designated PMX030, PMX031 the sequences of these antibodies are set out in Table 2.

Family 4 comprises antibodies generated from heavy chain V-gene IGHV3-8 and light chain V-gene IGLV3-24. Examples of antibodies in Family 4 include antibodies designated PMX035, PMX037, PMX040, PMX048, PMX049, PMX065 the sequences of these antibodies are set out in Table 2.

Family 5 comprises antibodies generated from heavy chain V-gene IGHV3-18 and light chain V-gene IGLV3-3. Examples of antibodies in Family 5 include antibodies designated PMX039, PMX041, PMX043, PMX044, PMX046, PMX050 PMX055, PMX056, PMX057, PMX058, PMX059, PMX062, PMX085 the sequences of these antibodies are set out in Table 2.

Family 6 comprises antibodies generated from heavy chain V-gene IGHV3-19 and light chain V-gene IGKV2-5. Examples of antibodies in Family 6 include antibodies designated PMX047 and PMX053, the sequences of these antibodies are set out in Table 2.

Family 7 comprises antibodies generated from heavy chain V-gene IGHV3-44 and light chain V-gene IGLV3-8. Examples of antibodies in Family 7 include antibodies designated PMX051, PMX088, the sequences of these antibodies are set out in Table 2.

Family 8 comprises antibodies generated from heavy chain V-gene IGHV3-19 and light chain V-gene IGKV2-4. Examples of antibodies in Family 8 include antibodies designated PMX052, the sequences of these antibodies are set out in Table 2.

Family 9 comprises antibodies generated from heavy chain V-gene IGHV3-5 and light chain V-gene IGLV3-3. Examples of antibodies in Family 9 include antibodies designated PMX060, PMX061, the sequences of these antibodies are set out in Table 2.

Family 10 comprises antibodies generated from heavy chain V-gene IGHV3-5 and light chain V-gene IGLV3-8. Examples of antibodies in Family 10 include antibodies designated PMX063, PMX086, PMX087, PMX094, PMX095, PMX096, PMX097, PMX098, PMX099, PMX100, PMX101, PMX102, PMX 103, PMX104, PMX105, PMX107, PMX108, PMX109, PMX110, PMX111, the sequences of these antibodies are set out in Table 2.

Family 11 comprises antibodies generated from heavy chain V-gene IGHV3-5 and light chain V-gene IGKV2-4. Examples of antibodies in Family 11 include antibodies designated PMX106, the sequences of these antibodies are set out in Table 2.

Family 12 comprises antibodies generated from heavy chain V-gene IGHV3-18 and light chain V-gene IGLV3-24. Examples of antibodies in Family 12 include antibodies designated PMX054, PMX084, PMX154, PMX293, PMX294 the sequences of these antibodies are set out in Table 2.

Family 13 comprises antibodies generated from heavy chain V-gene IGHV3-19 and light chain V-gene IGLV3-21. Examples of antibodies in Family 13 include antibodies designated PMX155, PMX156, the sequences of these antibodies are set out in Table 2.

Family 14 comprises antibodies generated from heavy chain V-gene IGHV3-41 and light chain V-gene IGLV3-24. Examples of antibodies in Family 14 include antibodies designated PMX291, PMX292, the sequences of these antibodies are set out in Table 2.

In one embodiment, the antibody or fragment may be selected from an antibody as shown in any of Families 1 to 14 or an antibody with at least 80% sequence identity thereto.

In one embodiment the antibody or fragment thereof comprises an antibody comprising a Family 1 sequence or a variant thereof. In one embodiment the antibody or fragment thereof comprises an antibody comprising a Family 2 sequence or a variant thereof. In one embodiment the antibody or fragment thereof comprises an antibody comprising a Family 3 sequence or a variant thereof. In one embodiment the antibody or fragment thereof comprises an antibody comprising a Family 4 sequence or a variant thereof. In one embodiment the antibody or fragment thereof comprises an antibody comprising a Family 5 sequence or a variant thereof. In one embodiment the antibody or fragment thereof comprises an antibody comprising a Family 6 sequence or a variant thereof. In one embodiment the antibody or fragment thereof comprises an antibody comprising a Family 7 sequence or a variant thereof. In one embodiment the antibody or fragment thereof comprises an antibody comprising a Family 8 sequence or a variant thereof. In one embodiment the antibody or fragment thereof comprises an antibody comprising a Family 9 sequence or a variant thereof. In one embodiment the antibody or fragment thereof comprises an antibody comprising a Family 10 sequence or a variant thereof. In one embodiment the antibody or fragment thereof comprises an antibody comprising a Family 11 sequence or a variant thereof. In one embodiment the antibody or fragment thereof comprises an antibody comprising a Family 12 sequence or a variant thereof. In one embodiment the antibody or fragment thereof comprises an antibody comprising a Family 13 sequence or a variant thereof. In one embodiment the antibody or fragment thereof comprises an antibody comprising a Family 14 sequence or a variant thereof.

In one embodiment the antibody that binds canine OX40L may be selected from one of the following antibodies
An antibody comprising a Family 1 sequence or a variant thereof, for example an antibody comprising a HC CDR1 comprising SEQ ID No: 15 or a sequence with at least 60%, 70%, 80%, 90% or 95% sequence identity thereto, a HC CDR2 comprising SEQ ID No: 16 or a sequence with at least 60%, 70%, 80%, 90% or 95% sequence identity thereto, a HC CDR3 comprising SEQ ID No: 17 or a sequence with at least 60%, 70%, 80%, 90% or 95% sequence identity thereto, a LC CDR1 comprising SEQ ID No: 18 or a sequence with at least 60%, 70%, 80%, 90% or 95% sequence identity thereto, a LC CDR2 comprising SEQ ID No: 19 or a sequence with at least 60%, 70%, 80%, 90% or 95% sequence identity thereto and a LC CDR3 comprising SEQ ID No: 20 or a sequence with at least 60%, 70%, 80%, 90% or 95% sequence identity thereto;
An antibody comprising a Family 2 sequence or a variant thereof, for example an antibody comprising a HC CDR1 comprising SEQ ID No: 45 or a sequence with at least 60%, 70%, 80%, 90% or 95% sequence identity thereto, a HC CDR2 comprising SEQ ID No: 46 or a sequence with at least 60%, 70%, 80%, 90% or 95% sequence identity thereto, a HC CDR3 comprising SEQ ID No: 47 or a sequence with at least 60%, 70%, 80%, 90% or 95% sequence identity thereto, a LC CDR1 comprising SEQ ID No: 48 or a sequence with at least 60%, 70%, 80%, 90% or 95% sequence identity thereto, a LC CDR2 comprising SEQ ID No: 49 or a sequence with at least 60%, 70%, 80%, 90% or 95% sequence identity thereto and a LC CDR3 comprising SEQ ID No: 50 or a sequence with at least 60%, 70%, 80%, 90% or 95% sequence identity thereto;
An antibody comprising a Family 3 sequence or a variant thereof, for example an antibody comprising a HC CDR1 comprising SEQ ID No: 65 or a sequence with at least 60%, 70%, 80%, 90% or 95% sequence identity thereto, a HC CDR2 comprising SEQ ID No: 66 or a sequence with at least 60%, 70%, 80%, 90% or 95% sequence identity thereto, a HC CDR3 comprising SEQ ID No: 67 or a sequence with at least 60%, 70%, 80%, 90% or 95% sequence identity thereto, a LC CDR1 comprising SEQ ID No: 68 or a sequence with at least 60%, 70%, 80%, 90% or 95% sequence identity thereto, a LC CDR2 comprising SEQ ID No: 69 or a sequence with at least 60%, 70%, 80%, 90% or 95% sequence identity thereto and a LC CDR3 comprising SEQ ID No: 70 or a sequence with at least 60%, 70%, 80%, 90% or 95% sequence identity thereto;
An antibody comprising a Family 4 sequence or a variant thereof, for example an antibody comprising a HC CDR1 comprising SEQ ID No: 115 or a sequence with at least 60%, 70%, 80%, 90% or 95% sequence identity thereto, a HC CDR2 comprising SEQ ID No: 116 or a sequence with at least 60%, 70%, 80%, 90% or 95% sequence identity thereto, a HC CDR3 comprising SEQ ID No: 117 or a sequence with at least 60%, 70%, 80%, 90% or 95% sequence identity thereto, a LC CDR1 comprising SEQ ID No: 118 or a sequence with at least 60%, 70%, 80%, 90% or 95% sequence identity thereto, a LC CDR2 comprising SEQ ID No: 119 or a sequence with at least 60%, 70%, 80%, 90% or 95% sequence identity thereto and a LC CDR3 comprising SEQ ID No: 120 or a sequence with at least 60%, 70%, 80%, 90% or 95% sequence identity thereto;
An antibody comprising a Family 5 sequence or a variant thereof, for example an antibody comprising a HC CDR1 comprising SEQ ID No: 155 or a sequence with at least 60%, 70%, 80%, 90% or 95% sequence identity thereto, a HC CDR2 comprising SEQ ID No: 156 or a sequence with at least 60%, 70%, 80%, 90% or 95% sequence identity thereto, a HC CDR3 comprising SEQ ID No: 157 or a sequence with at least 60%, 70%, 80%, 90% or 95% sequence identity thereto, a LC CDR1 comprising SEQ ID No: 158 or a sequence with at least 60%, 70%, 80%, 90% or 95% sequence identity thereto, a LC CDR2 comprising SEQ ID No: 159 or a sequence with at least 60%, 70%, 80%, 90% or 95% sequence identity thereto and a LC CDR3 comprising SEQ ID No: 160 or a sequence with at least 60%, 70%, 80%, 90% or 95% sequence identity thereto;
An antibody comprising a Family 6 sequence or a variant thereof, for example an antibody comprising a HC CDR1 comprising SEQ ID No: 235 or a sequence with at least 60%, 70%, 80%, 90% or 95% sequence identity thereto, a HC CDR2 comprising SEQ ID No: 236 or a sequence with at least 60%, 70%, 80%, 90% or 95% sequence identity thereto, a HC CDR3 comprising SEQ ID No: 237 or a sequence with at least 60%, 70%, 80%, 90% or 95% sequence identity thereto, a LC CDR1 comprising SEQ ID No: 238 or a sequence with at least 60%, 70%, 80%, 90% or 95% sequence identity thereto, a LC CDR2 comprising SEQ ID No: 239 or a sequence with at least 60%, 70%, 80%, 90% or 95% sequence identity thereto and a LC CDR3 comprising SEQ ID No: 240 or a sequence with at least 60%, 70%, 80%, 90% or 95% sequence identity thereto;
An antibody comprising a Family 7 sequence or a variant thereof, for example an antibody comprising a HC CDR1 comprising SEQ ID No: 275 or a sequence with at least 60%, 70%, 80%, 90% or 95% sequence identity thereto, a HC CDR2 comprising SEQ ID No: 276 or a sequence with at least 60%, 70%, 80%, 90% or 95% sequence identity thereto, a HC CDR3 comprising SEQ ID No: 277 or a sequence with at least 60%, 70%, 80%, 90% or 95% sequence identity thereto, a LC CDR1 comprising SEQ ID No: 278 or a sequence with at least 60%, 70%, 80%, 90% or 95% sequence identity thereto, a LC CDR2 comprising SEQ ID No: 279 or a sequence with at least 60%, 70%, 80%, 90% or 95% sequence identity thereto and a LC CDR3 comprising SEQ ID No: 280 or a sequence with at least 60%, 70%, 80%, 90% or 95% sequence identity thereto;
An antibody comprising a Family 8 sequence or a variant thereof, for example an antibody comprising a HC CDR1 comprising SEQ ID No: 285 or a sequence with at least 60%, 70%, 80%, 90% or 95% sequence identity thereto, a HC CDR2 comprising SEQ ID No: 286 or a sequence with at least 60%, 70%, 80%, 90% or 95% sequence identity thereto, a HC CDR3 comprising SEQ ID No: 287 or a sequence with at least 60%, 70%, 80%, 90% or 95% sequence identity thereto, a LC CDR1 comprising SEQ ID No: 288 or a sequence with at least 60%, 70%, 80%, 90% or 95% sequence identity thereto, a LC CDR2 comprising SEQ ID No: 289 or a sequence with at least 60%, 70%, 80%, 90% or 95% sequence identity thereto and a LC CDR3 comprising SEQ ID No: 290 or a sequence with at least 60%, 70%, 80%, 90% or 95% sequence identity thereto;
An antibody comprising a Family 9 sequence or a variant thereof, for example an antibody comprising a HC CDR1 comprising SEQ ID No: 365 or a sequence with at least 60%, 70%, 80%, 90% or 95% sequence identity thereto, a HC CDR2 comprising SEQ ID No: 366 or a sequence with at least 60%, 70%, 80%, 90% or 95% sequence identity thereto, a HC CDR3 comprising SEQ ID No: 367 or a sequence with at least 60%, 70%, 80%, 90% or 95% sequence identity thereto, a LC CDR1 comprising SEQ ID No: 368 or a sequence with at least 60%, 70%, 80%, 90% or 95% sequence identity thereto, a LC CDR2 comprising SEQ ID No: 369 or a sequence with at least 60%, 70%, 80%, 90% or 95% sequence identity thereto and a LC CDR3 comprising SEQ ID No: 370 or a sequence with at least 60%, 70%, 80%, 90% or 95% sequence identity thereto;
An antibody comprising a Family 10 sequence or a variant thereof, for example an antibody comprising a HC CDR1 comprising SEQ ID No: 575 or a sequence with at least 60%, 70%, 80%, 90% or 95% sequence identity thereto, a HC CDR2 comprising SEQ ID No: 576 or a sequence with at least 60%, 70%, 80%, 90% or 95% sequence identity thereto, a HC CDR3 comprising SEQ ID No: 577 or a sequence with at least 60%, 70%, 80%, 90% or 95% sequence identity thereto, a LC CDR1 comprising SEQ ID No: 578 or a sequence with at least 60%, 70%, 80%, 90% or 95% sequence identity thereto, a LC CDR2 comprising SEQ ID No: 579 or a sequence with at least 60%, 70%, 80%, 90% or 95% sequence identity thereto and a LC CDR3 comprising SEQ ID No: 580 or a sequence with at least 60%, 70%, 80%, 90% or 95% sequence identity thereto;
An antibody comprising a Family 10 sequence or a variant thereof, for example an antibody comprising a HC CDR1 comprising SEQ ID No: 395 or a sequence with at least 60%, 70%, 80%, 90% or 95% sequence identity thereto, a HC CDR2 comprising SEQ ID No: 396 or a sequence with at least 60%, 70%, 80%, 90% or 95% sequence identity thereto, a HC CDR3 comprising SEQ ID No: 397 or a sequence with at least 60%, 70%, 80%, 90% or 95% sequence identity thereto, a LC CDR1 comprising SEQ ID No: 398 or a sequence with at least 60%, 70%, 80%, 90% or 95% sequence identity thereto, a LC CDR2 comprising SEQ ID No: 399 or a sequence with at least 60%, 70%, 80%, 90% or 95% sequence identity thereto and a LC CDR3 comprising SEQ ID No: 400 or a sequence with at least 60%, 70%, 80%, 90% or 95% sequence identity thereto;
An antibody comprising a Family 11 sequence or a variant thereof, for example an antibody comprising a HC CDR1 comprising SEQ ID No: 665 or a sequence with at least 60%, 70%, 80%, 90% or 95% sequence identity thereto, a HC CDR2 comprising SEQ ID No: 666 or a sequence with at least 60%, 70%, 80%, 90% or 95% sequence identity thereto, a HC CDR3 comprising SEQ ID No: 667 or a sequence with at least 60%, 70%, 80%, 90% or 95% sequence identity thereto, a LC CDR1 comprising SEQ ID No: 668 or a sequence with at least 60%, 70%, 80%, 90% or 95% sequence identity thereto, a LC CDR2 comprising SEQ ID No: 669 or a sequence with at least 60%, 70%, 80%, 90% or 95% sequence identity thereto and a LC CDR3 comprising SEQ ID No: 670 or a sequence with at least 60%, 70%, 80%, 90% or 95% sequence identity thereto.
An antibody comprising a Family 12 sequence or a variant thereof, for example an antibody comprising a HC CDR1 comprising SEQ ID No: 741 or a sequence with at least 60%, 70%, 80%, 90% or 95% sequence identity thereto, a HC CDR2 comprising SEQ ID No: 742 or a sequence with at least 60%, 70%, 80%, 90% or 95% sequence identity thereto, a HC CDR3 comprising SEQ ID No: 743 or a sequence with at least 60%, 70%, 80%, 90% or 95% sequence identity thereto, a LC CDR1 comprising SEQ ID No: 744 or a sequence with at least 60%, 70%, 80%, 90% or 95% sequence identity thereto, a LC CDR2 comprising SEQ ID No: 745 or a sequence with at least 60%, 70%, 80%, 90% or 95% sequence identity thereto and a LC CDR3 comprising SEQ ID No: 746 or a sequence with at least 60%, 70%, 80%, 90% or 95% sequence identity thereto.
An antibody comprising a Family 13 sequence or a variant thereof, for example an antibody comprising a HC CDR1 comprising SEQ ID No: 751 or a sequence with at least 60%, 70%, 80%, 90% or 95% sequence identity thereto, a HC CDR2 comprising SEQ ID No: 752 or a sequence with at least 60%, 70%, 80%, 90% or 95% sequence identity thereto, a HC CDR3 comprising SEQ ID No: 753 or a sequence with at least 60%, 70%, 80%, 90% or 95% sequence identity thereto, a LC CDR1 comprising SEQ ID No: 754 or a sequence with at least 60%, 70%, 80%, 90% or 95% sequence identity thereto, a LC CDR2 comprising SEQ ID No: 755 or a sequence with at least 60%, 70%, 80%, 90% or 95% sequence identity thereto and a LC CDR3 comprising SEQ ID No: 756 or a sequence with at least 60%, 70%, 80%, 90% or 95% sequence identity thereto.
An antibody comprising a Family 14 sequence or a variant thereof, for example an antibody comprising a HC CDR1 comprising SEQ ID No: 771 or a sequence with at least 60%, 70%, 80%, 90% or 95% sequence identity thereto, a HC CDR2 comprising SEQ ID No: 772 or a sequence with at least 60%, 70%, 80%, 90% or 95% sequence identity thereto, a HC CDR3 comprising SEQ ID No: 773 or a sequence with at least 60%, 70%, 80%, 90% or 95% sequence identity thereto, a LC CDR1 comprising SEQ ID No: 774 or a sequence with at least 60%, 70%, 80%, 90% or 95% sequence identity thereto, a LC CDR2 comprising SEQ ID No: 775 or a sequence with at least 60%, 70%, 80%, 90% or 95% sequence identity thereto and a LC CDR3 comprising SEQ ID No: 776 or a sequence with at least 60%, 70%, 80%, 90% or 95% sequence identity thereto.

In one embodiment, the antibody that binds canine OX40L may be selected from one of the following antibodies:
An antibody comprising a HC CDR1 comprising SEQ ID No: 15 or a sequence with at least 80% sequence identity thereto, a HC CDR2 comprising SEQ ID No: 16 or a sequence with at least 80% sequence identity thereto, a HC CDR3 comprising SEQ ID No: 17 or a sequence with at least 80% sequence identity thereto, a LC CDR1 comprising SEQ ID No: 18 or a sequence with at least 80% sequence identity thereto, a LC CDR2 comprising SEQ ID No: 19 or a sequence with at least 80% sequence identity thereto and a LC CDR3 comprising SEQ ID No: 20 or a sequence with at least 80% sequence identity thereto;
An antibody comprising a HC CDR1 comprising SEQ ID No: 25 or a sequence with at least 80% sequence identity thereto, a HC CDR2 comprising SEQ ID No: 26 or a sequence with at least 80% sequence identity thereto, a HC CDR3 comprising SEQ ID No: 27 or a sequence with at least 80% sequence identity thereto, a LC CDR1 comprising SEQ ID No: 28 or a sequence with at least 80% sequence identity thereto, a LC CDR2 comprising SEQ ID No: 29 or a sequence with at least 80% sequence identity thereto and a LC CDR3 comprising SEQ ID No: 30 or a sequence with at least 80% sequence identity thereto;
An antibody comprising a HC CDR1 comprising SEQ ID No: 35 or a sequence with at least 80% sequence identity thereto, a HC CDR2 comprising SEQ ID No: 36 or a sequence with at least 80% sequence identity thereto, a HC CDR3 comprising SEQ ID No: 37 or a sequence with at least 80% sequence identity thereto, a LC CDR1 comprising SEQ ID No: 38 or a sequence with at least 80% sequence identity thereto, a LC CDR2 comprising SEQ ID No: 39 or a sequence with at least 80% sequence identity thereto and a LC CDR3 comprising SEQ ID No: 40 or a sequence with at least 80% sequence identity thereto;
An antibody comprising a HC CDR1 comprising SEQ ID No: 45 or a sequence with at least 80% sequence identity thereto, a HC CDR2 comprising SEQ ID No: 46 or a sequence with at least 80% sequence identity thereto, a HC CDR3 comprising SEQ ID No: 47 or a sequence with at least 80% sequence identity thereto, a LC CDR1 comprising SEQ ID No: 48 or a sequence with at least 80% sequence identity thereto, a LC CDR2 comprising SEQ ID No: 49 or a sequence with at least 80% sequence identity thereto and a LC CDR3 comprising SEQ ID No: 50 or a sequence with at least 80% sequence identity thereto;
An antibody comprising a HC CDR1 comprising SEQ ID No: 55 or a sequence with at least 80% sequence identity thereto, a HC CDR2 comprising SEQ ID No: 56 or a sequence with at least 80% sequence identity thereto, a HC CDR3 comprising SEQ ID No: 57, or a sequence with at least 80% sequence identity thereto, a LC CDR1 comprising SEQ ID No: 58 or a sequence with at least 80% sequence identity thereto, a LC CDR2 comprising SEQ ID No: 59 or a sequence with at least 80% sequence identity thereto and a LC CDR3 comprising SEQ ID No: 60 or a sequence with at least 80% sequence identity thereto;
An antibody comprising a HC CDR1 comprising SEQ ID No: 65 or a sequence with at least 80% sequence identity thereto, a HC CDR2 comprising SEQ ID No: 66 or a sequence with at least 80% sequence identity thereto, a HC CDR3 comprising SEQ ID No: 67 or a sequence with at least 80% sequence identity thereto, a LC CDR1 comprising SEQ ID No: 68 or a sequence with at least 80% sequence identity thereto, a LC CDR2 comprising SEQ ID No: 69 or a sequence with at least 80% sequence identity thereto and a LC CDR3 comprising SEQ ID No: 70 or a sequence with at least 80% sequence identity thereto;
An antibody comprising a HC CDR1 comprising SEQ ID No: 75 or a sequence with at least 80% sequence identity thereto, a HC CDR2 comprising SEQ ID No: 76 or a sequence with at least 80% sequence identity thereto, a HC CDR3 comprising SEQ ID No: 77 or a sequence with at least 80% sequence identity thereto, a LC CDR1 comprising SEQ ID No: 78 or a sequence with at least 80% sequence identity thereto, a LC CDR2 comprising SEQ ID No: 79 or a sequence with at least 80% sequence identity thereto and a LC CDR3 comprising SEQ ID No: 80 or a sequence with at least 80% sequence identity thereto;
An antibody comprising a HC CDR1 comprising SEQ ID No: 85 or a sequence with at least 80% sequence identity thereto, a HC CDR2 comprising SEQ ID No: 86 or a sequence with at least 80% sequence identity thereto, a HC CDR3 comprising SEQ ID No: 87 or a sequence with at least 80% sequence identity thereto, a LC CDR1 comprising SEQ ID No: 88 or a sequence with at least 80% sequence identity thereto, a LC CDR2 comprising SEQ ID No: 89 or a sequence with at least 80% sequence identity thereto and a LC CDR3 comprising SEQ ID No: 90 or a sequence with at least 80% sequence identity thereto;
An antibody comprising a HC CDR1 comprising SEQ ID No: 95 or a sequence with at least 80% sequence identity thereto, a HC CDR2 comprising SEQ ID No: 96 or a sequence with at least 80% sequence identity thereto, a HC CDR3 comprising SEQ ID No: 97 or a sequence with at least 80% sequence identity thereto, a LC CDR1 comprising SEQ ID No: 98 or a sequence with at least 80% sequence identity thereto, a LC CDR2 comprising SEQ ID No: 99 or a sequence with at least 80% sequence identity thereto and a LC CDR3 comprising SEQ ID No: 100 or a sequence with at least 80% sequence identity thereto;
An antibody comprising a HC CDR1 comprising SEQ ID No: 105 or a sequence with at least 80% sequence identity thereto, a HC CDR2 comprising SEQ ID No: 106 or a sequence with at least 80% sequence identity thereto, a HC CDR3 comprising SEQ ID No: 107 or a sequence with at least 80% sequence identity thereto, a LC CDR1 comprising SEQ ID No: 108 or a sequence with at least 80% sequence identity thereto, a LC CDR2 comprising SEQ ID No: 109 or a sequence with at least 80% sequence identity thereto and a LC CDR3 comprising SEQ ID No: 110 or a sequence with at least 80% sequence identity thereto;
An antibody comprising a HC CDR1 comprising SEQ ID No: 115 or a sequence with at least 80% sequence identity thereto, a HC CDR2 comprising SEQ ID No: 116 or a sequence with at least 80% sequence identity thereto, a HC CDR3 comprising SEQ ID No: 117 or a sequence with at least 80% sequence identity thereto, a LC CDR1 comprising SEQ ID No: 118 or a sequence with at least 80% sequence identity thereto, a LC CDR2 comprising SEQ ID No: 119 or a sequence with at least 80% sequence identity thereto and a LC CDR3 comprising SEQ ID No: 120 or a sequence with at least 80% sequence identity thereto;
An antibody comprising a HC CDR1 comprising SEQ ID No: 125 or a sequence with at least 80% sequence identity thereto, a HC CDR2 comprising SEQ ID No: 126 or a sequence with at least 80% sequence identity thereto, a HC CDR3 comprising SEQ ID No: 127 or a sequence with at least 80% sequence identity thereto, a LC CDR1 comprising SEQ ID No: 128 or a sequence with at least 80% sequence identity thereto, a LC CDR2 comprising SEQ ID No: 129 or a sequence with at least 80% sequence identity thereto and a LC CDR3 comprising SEQ ID No: 130 or a sequence with at least 80% sequence identity thereto; or
An antibody comprising a HC CDR1 comprising SEQ ID No: 135 or a sequence with at least 80% sequence identity thereto, a HC CDR2 comprising SEQ ID No: 136 or a sequence with at least 80% sequence identity thereto, a HC CDR3 comprising SEQ ID No: 137 or a sequence with at least 80% sequence identity thereto, a LC CDR1 comprising SEQ ID No: 138 or a sequence with at least 80% sequence identity thereto, a LC CDR2 comprising SEQ ID No: 139 or a sequence with at least 80% sequence identity thereto and a LC CDR3 comprising SEQ ID No: 140 or a sequence with at least 80% sequence identity thereto.
An antibody comprising a HC CDR1 comprising SEQ ID No: 145 or a sequence with at least 80% sequence identity thereto, a HC CDR2 comprising SEQ ID No: 146 or a sequence with at least 80% sequence identity thereto, a HC CDR3 comprising SEQ ID No: 147 or a sequence with at least 80% sequence identity thereto, a LC CDR1 comprising SEQ ID No: 148 or a sequence with at least 80% sequence identity thereto, a LC CDR2 comprising SEQ ID No: 149 or a sequence with at least 80% sequence identity thereto and a LC CDR3 comprising SEQ ID No: 150 or a sequence with at least 80% sequence identity thereto;
An antibody comprising a HC CDR1 comprising SEQ ID No: 155 or a sequence with at least 80% sequence identity thereto, a HC CDR2 comprising SEQ ID No: 156 or a sequence with at least 80% sequence identity thereto, a HC CDR3 comprising SEQ ID No: 157 or a sequence with at least 80% sequence identity thereto, a LC CDR1 comprising SEQ ID No: 158 or a sequence with at least 80% sequence identity thereto, a LC CDR2 comprising SEQ ID No: 159 or a sequence with at least 80% sequence identity thereto and a LC CDR3 comprising SEQ ID No: 160 or a sequence with at least 80% sequence identity thereto;
An antibody comprising a HC CDR1 comprising SEQ ID No: 165 or a sequence with at least 80% sequence identity thereto, a HC CDR2 comprising SEQ ID No: 166 or a sequence with at least 80% sequence identity thereto, a HC CDR3 comprising SEQ ID No: 167 or a sequence with at least 80% sequence identity thereto, a LC CDR1 comprising SEQ ID No: 168 or a sequence with at least 80% sequence identity thereto, a LC CDR2 comprising SEQ ID No: 169 or a sequence with at least 80% sequence identity thereto and a LC CDR3 comprising SEQ ID No: 170 or a sequence with at least 80% sequence identity thereto;
An antibody comprising a HC CDR1 comprising SEQ ID No: 175 or a sequence with at least 80% sequence identity thereto, a HC CDR2 comprising SEQ ID No: 176 or a sequence with at least 80% sequence identity thereto, a HC CDR3 comprising SEQ ID No: 177 or a sequence with at least 80% sequence identity thereto, a LC CDR1 comprising SEQ ID No: 178 or a sequence with at least 80% sequence identity thereto, a LC CDR2 comprising SEQ ID No: 179 or a sequence with at least 80% sequence identity thereto and a LC CDR3 comprising SEQ ID No: 180 or a sequence with at least 80% sequence identity thereto;
An antibody comprising a HC CDR1 comprising SEQ ID No: 185 or a sequence with at least 80% sequence identity thereto, a HC CDR2 comprising SEQ ID No: 186 or a sequence with at least 80% sequence identity thereto, a HC CDR3 comprising SEQ ID No: 187 or a sequence with at least 80% sequence identity thereto, a LC CDR1 comprising SEQ ID No: 188 or a sequence with at least 80% sequence identity thereto, a LC CDR2 comprising SEQ ID No: 189 or a sequence with at least 80% sequence identity thereto and a LC CDR3 comprising SEQ ID No: 190 or a sequence with at least 80% sequence identity thereto;
An antibody comprising a HC CDR1 comprising SEQ ID No: 195 or a sequence with at least 80% sequence identity thereto, a HC CDR2 comprising SEQ ID No: 196 or a sequence with at least 80% sequence identity thereto, a HC CDR3 comprising SEQ ID No: 197 or a sequence with at least 80% sequence identity thereto, a LC CDR1 comprising SEQ ID No: 198 or a sequence with at least 80% sequence identity thereto, a LC CDR2 comprising SEQ ID No: 199 or a sequence with at least 80% sequence identity thereto and a LC CDR3 comprising SEQ ID No: 200 or a sequence with at least 80% sequence identity thereto;
An antibody comprising a HC CDR1 comprising SEQ ID No: 205 or a sequence with at least 80% sequence identity thereto, a HC CDR2 comprising SEQ ID No: 206 or a sequence with at least 80% sequence identity thereto, a HC CDR3 comprising SEQ ID No: 207 or a sequence with at least 80% sequence identity thereto, a LC CDR1 comprising SEQ ID No: 208 or a sequence with at least 80% sequence identity thereto, a LC CDR2 comprising SEQ ID No: 209 or a sequence with at least 80% sequence identity thereto and a LC CDR3 comprising SEQ ID No: 210 or a sequence with at least 80% sequence identity thereto;
An antibody comprising a HC CDR1 comprising SEQ ID No: 215 or a sequence with at least 80% sequence identity thereto, a HC CDR2 comprising SEQ ID No: 216 or a sequence with at least 80% sequence identity thereto, a HC CDR3 comprising SEQ ID No: 217 or a sequence with at least 80% sequence identity thereto, a LC CDR1 comprising SEQ ID No: 218 or a sequence with at least 80% sequence identity thereto, a LC CDR2 comprising SEQ ID No: 219 or a sequence with at least 80% sequence identity thereto and a LC CDR3 comprising SEQ ID No: 220 or a sequence with at least 80% sequence identity thereto;
An antibody comprising a HC CDR1 comprising SEQ ID No: 225 or a sequence with at least 80% sequence identity thereto, a HC CDR2 comprising SEQ ID No: 226 or a sequence with at least 80% sequence identity thereto, a HC CDR3 comprising SEQ ID No: 227 or a sequence with at least 80% sequence identity thereto, a LC CDR1 comprising SEQ ID No: 228 or a sequence with at least 80% sequence identity thereto, a LC CDR2 comprising SEQ ID No: 229 or a sequence with at least 80% sequence identity thereto and a LC CDR3 comprising SEQ ID No: 230 or a sequence with at least 80% sequence identity thereto;
An antibody comprising a HC CDR1 comprising SEQ ID No: 235 or a sequence with at least 80% sequence identity thereto, a HC CDR2 comprising SEQ ID No: 236 or a sequence with at least 80% sequence identity thereto, a HC CDR3 comprising SEQ ID No: 237 or a sequence with at least 80% sequence identity thereto, a LC CDR1 comprising SEQ ID No: 238 or a sequence with at least 80% sequence identity thereto, a LC CDR2 comprising SEQ ID No: 239 or a sequence with at least 80% sequence identity thereto and a LC CDR3 comprising SEQ ID No: 240 or a sequence with at least 80% sequence identity thereto;
An antibody comprising a HC CDR1 comprising SEQ ID No: 245 or a sequence with at least 80% sequence identity thereto, a HC CDR2 comprising SEQ ID No: 246 or a sequence with at least 80% sequence identity thereto, a HC CDR3 comprising SEQ ID No: 247 or a sequence with at least 80% sequence identity thereto, a LC CDR1 comprising SEQ ID No: 248 or a sequence with at least 80% sequence identity thereto, a LC CDR2 comprising SEQ ID No: 249 or a sequence with at least 80% sequence identity thereto and a LC CDR3 comprising SEQ ID No: 250 or a sequence with at least 80% sequence identity thereto;
An antibody comprising a HC CDR1 comprising SEQ ID No: 255 or a sequence with at least 80% sequence identity thereto, a HC CDR2 comprising SEQ ID No: 256 or a sequence with at least 80% sequence identity thereto, a HC CDR3 comprising SEQ ID No: 257 or a sequence with at least 80% sequence identity thereto, a LC CDR1 comprising SEQ ID No: 258 or a sequence with at least 80% sequence identity thereto, a LC CDR2 comprising SEQ ID No: 259 or a sequence with at least 80% sequence identity thereto and a LC CDR3 comprising SEQ ID No: 260 or a sequence with at least 80% sequence identity thereto;
An antibody comprising a HC CDR1 comprising SEQ ID No: 265 or a sequence with at least 80% sequence identity thereto, a HC CDR2 comprising SEQ ID No: 266 or a sequence with at least 80% sequence identity thereto, a HC CDR3 comprising SEQ ID No: 267 or a sequence with at least 80% sequence identity thereto, a LC CDR1 comprising SEQ ID No: 268 or a sequence with at least 80% sequence identity thereto, a LC CDR2 comprising SEQ ID No: 269 or a sequence with at least 80% sequence identity thereto and a LC CDR3 comprising SEQ ID No: 270 or a sequence with at least 80% sequence identity thereto;
An antibody comprising a HC CDR1 comprising SEQ ID No: 275 or a sequence with at least 80% sequence identity thereto, a HC CDR2 comprising SEQ ID No: 276 or a sequence with at least 80% sequence identity thereto, a HC CDR3 comprising SEQ ID No: 277 or a sequence with at least 80% sequence identity thereto, a LC CDR1 comprising SEQ ID No: 278 or a sequence with at least 80% sequence identity thereto, a LC CDR2 comprising SEQ ID No: 279 or a sequence with at least 80% sequence identity thereto and a LC CDR3 comprising SEQ ID No: 280 or a sequence with at least 80% sequence identity thereto;
An antibody comprising a HC CDR1 comprising SEQ ID No: 285 or a sequence with at least 80% sequence identity thereto, a HC CDR2 comprising SEQ ID No: 286 or a sequence with at least 80% sequence identity thereto, a HC CDR3 comprising SEQ ID No: 287 or a sequence with at least 80% sequence identity thereto, a LC CDR1 comprising SEQ ID No: 288 or a sequence with at least 80% sequence identity thereto, a LC CDR2 comprising SEQ ID No: 289 or a sequence with at least 80% sequence identity thereto and a LC CDR3 comprising SEQ ID No: 290 or a sequence with at least 80% sequence identity thereto;
An antibody comprising a HC CDR1 comprising SEQ ID No: 295 or a sequence with at least 80% sequence identity thereto, a HC CDR2 comprising SEQ ID No: 296 or a sequence with at least 80% sequence identity thereto, a HC CDR3 comprising SEQ ID No: 297 or a sequence with at least 80% sequence identity thereto, a LC CDR1 comprising SEQ ID No: 298 or a sequence with at least 80% sequence identity thereto, a LC CDR2 comprising SEQ ID No: 299 or a sequence with at least 80% sequence identity thereto and a LC CDR3 comprising SEQ ID No: 300 or a sequence with at least 80% sequence identity thereto;
An antibody comprising a HC CDR1 comprising SEQ ID No: 305 or a sequence with at least 80% sequence identity thereto, a HC CDR2 comprising SEQ ID No: 306 or a sequence with at least 80% sequence identity thereto, a HC CDR3 comprising SEQ ID No: 307 or a sequence with at least 80% sequence identity thereto, a LC CDR1 comprising SEQ ID No: 308 or a sequence with at least 80% sequence identity thereto, a LC CDR2 comprising SEQ ID No: 309 or a sequence with at least 80% sequence identity thereto and a LC CDR3 comprising SEQ ID No: 310 or a sequence with at least 80% sequence identity thereto;
An antibody comprising a HC CDR1 comprising SEQ ID No: 315 or a sequence with at least 80% sequence identity thereto, a HC CDR2 comprising SEQ ID No: 316 or a sequence with at least 80% sequence identity thereto, a HC CDR3 comprising SEQ ID No: 317 or a sequence with at least 80% sequence identity thereto, a LC CDR1 comprising SEQ ID No: 318 or a sequence with at least 80% sequence identity thereto, a LC CDR2 comprising SEQ ID No: 319 or a sequence with at least 80% sequence identity thereto and a LC CDR3 comprising SEQ ID No: 320 or a sequence with at least 80% sequence identity thereto;
An antibody comprising a HC CDR1 comprising SEQ ID No: 325 or a sequence with at least 80% sequence identity thereto, a HC CDR2 comprising SEQ ID No: 326 or a sequence with at least 80% sequence identity thereto, a HC CDR3 comprising SEQ ID No: 327 or a sequence with at least 80% sequence identity thereto, a LC CDR1 comprising SEQ ID No: 328 or a sequence with at least 80% sequence identity thereto, a LC CDR2 comprising SEQ ID No: 329 or a sequence with at least 80% sequence identity thereto and a LC CDR3 comprising SEQ ID No: 330 or a sequence with at least 80% sequence identity thereto;
An antibody comprising a HC CDR1 comprising SEQ ID No: 335 or a sequence with at least 80% sequence identity thereto, a HC CDR2 comprising SEQ ID No: 336 or a sequence with at least 80% sequence identity thereto, a HC CDR3 comprising SEQ ID No: 337 or a sequence with at least 80% sequence identity thereto, a LC CDR1 comprising SEQ ID No: 338 or a sequence with at least 80% sequence identity thereto, a LC CDR2 comprising SEQ ID No: 339 or a sequence with at least 80% sequence identity thereto and a LC CDR3 comprising SEQ ID No: 340 or a sequence with at least 80% sequence identity thereto;
An antibody comprising a HC CDR1 comprising SEQ ID No: 345 or a sequence with at least 80% sequence identity thereto, a HC CDR2 comprising SEQ ID No: 346 or a sequence with at least 80% sequence identity thereto, a HC CDR3 comprising SEQ ID No: 347 or a sequence with at least 80% sequence identity thereto, a LC CDR1 comprising SEQ ID No: 348 or a sequence with at least 80% sequence identity thereto, a LC CDR2 comprising SEQ ID No: 349 or a sequence with at least 80% sequence identity thereto and a LC CDR3 comprising SEQ ID No: 350 or a sequence with at least 80% sequence identity thereto;
An antibody comprising a HC CDR1 comprising SEQ ID No: 355 or a sequence with at least 80% sequence identity thereto, a HC CDR2 comprising SEQ ID No: 356 or a sequence with at least 80% sequence identity thereto, a HC CDR3 comprising SEQ ID No: 357 or a sequence with at least 80% sequence identity thereto, a LC CDR1 comprising SEQ ID No: 358 or a sequence with at least 80% sequence identity thereto, a LC CDR2 comprising SEQ ID No: 359 or a sequence with at least 80% sequence identity thereto and a LC CDR3 comprising SEQ ID No: 360 or a sequence with at least 80% sequence identity thereto;
An antibody comprising a HC CDR1 comprising SEQ ID No: 365 or a sequence with at least 80% sequence identity thereto, a HC CDR2 comprising SEQ ID No: 366 or a sequence with at least 80% sequence identity thereto, a HC CDR3 comprising SEQ ID No: 367 or a sequence with at least 80% sequence identity thereto, a LC CDR1 comprising SEQ ID No: 368 or a sequence with at least 80% sequence identity thereto, a LC CDR2 comprising SEQ ID No: 369 or a sequence with at least 80% sequence identity thereto and a LC CDR3 comprising SEQ ID No: 370 or a sequence with at least 80% sequence identity thereto;
An antibody comprising a HC CDR1 comprising SEQ ID No: 375 or a sequence with at least 80% sequence identity thereto, a HC CDR2 comprising SEQ ID No: 376 or a sequence with at least 80% sequence identity thereto, a HC CDR3 comprising SEQ ID No: 377 or a sequence with at least 80% sequence identity thereto, a LC CDR1 comprising SEQ ID No: 378 or a sequence with at least 80% sequence identity thereto, a LC CDR2 comprising SEQ ID No: 379 or a sequence with at least 80% sequence identity thereto and a LC CDR3 comprising SEQ ID No: 380 or a sequence with at least 80% sequence identity thereto;
An antibody comprising a HC CDR1 comprising SEQ ID No: 385 or a sequence with at least 80% sequence identity thereto, a HC CDR2 comprising SEQ ID No: 386 or a sequence with at least 80% sequence identity thereto, a HC CDR3 comprising SEQ ID No: 387 or a sequence with at least 80% sequence identity thereto, a LC CDR1 comprising SEQ ID No: 388 or a sequence with at least 80% sequence identity thereto, a LC CDR2 comprising SEQ ID No: 389 or a sequence with at least 80% sequence identity thereto and a LC CDR3 comprising SEQ ID No: 390 or a sequence with at least 80% sequence identity thereto;
An antibody comprising a HC CDR1 comprising SEQ ID No: 395 or a sequence with at least 80% sequence identity thereto, a HC CDR2 comprising SEQ ID No: 396 or a sequence with at least 80% sequence identity thereto, a HC CDR3 comprising SEQ ID No: 397 or a sequence with at least 80% sequence identity thereto, a LC CDR1 comprising SEQ ID No: 398 or a sequence with at least 80% sequence identity thereto, a LC CDR2 comprising SEQ ID No: 399 or a sequence with at least 80% sequence identity thereto and a LC CDR3 comprising SEQ ID No: 400 or a sequence with at least 80% sequence identity thereto;
An antibody comprising a HC CDR1 comprising SEQ ID No: 405 or a sequence with at least 80% sequence identity thereto, a HC CDR2 comprising SEQ ID No: 406 or a sequence with at least 80% sequence identity thereto, a HC CDR3 comprising SEQ ID No: 407 or a sequence with at least 80% sequence identity thereto, a LC CDR1 comprising SEQ ID No: 408 or a sequence with at least 80% sequence identity thereto, a LC CDR2 comprising SEQ ID No: 409 or a sequence with at least 80% sequence identity thereto and a LC CDR3 comprising SEQ ID No: 410 or a sequence with at least 80% sequence identity thereto;
An antibody comprising a HC CDR1 comprising SEQ ID No: 415 or a sequence with at least 80% sequence identity thereto, a HC CDR2 comprising SEQ ID No: 416 or a sequence with at least 80% sequence identity thereto, a HC CDR3 comprising SEQ ID No: 417 or a sequence with at least 80% sequence identity thereto, a LC CDR1 comprising SEQ ID No: 418 or a sequence with at least 80% sequence identity thereto, a LC CDR2 comprising SEQ ID No: 419 or a sequence with at least 80% sequence identity thereto and a LC CDR3 comprising SEQ ID No: 420 or a sequence with at least 80% sequence identity thereto;
An antibody comprising a HC CDR1 comprising SEQ ID No: 425 or a sequence with at least 80% sequence identity thereto, a HC CDR2 comprising SEQ ID No: 426 or a sequence with at least 80% sequence identity thereto, a HC CDR3 comprising SEQ ID No: 427 or a sequence with at least 80% sequence identity thereto, a LC CDR1 comprising SEQ ID No: 428 or a sequence with at least 80% sequence identity thereto, a LC CDR2 comprising SEQ ID No: 429 or a sequence with at least 80% sequence identity thereto and a LC CDR3 comprising SEQ ID No: 430 or a sequence with at least 80% sequence identity thereto;
An antibody comprising a HC CDR1 comprising SEQ ID No: 435 or a sequence with at least 80% sequence identity thereto, a HC CDR2 comprising SEQ ID No: 436 or a sequence with at least 80% sequence identity thereto, a HC CDR3 comprising SEQ ID No: 437 or a sequence with at least 80% sequence identity thereto, a LC CDR1 comprising SEQ ID No: 438 or a sequence with at least 80% sequence identity thereto, a LC CDR2 comprising SEQ ID No: 439 or a sequence with at least 80% sequence identity thereto and a LC CDR3 comprising SEQ ID No: 440 or a sequence with at least 80% sequence identity thereto;
An antibody comprising a HC CDR1 comprising SEQ ID No: 445 or a sequence with at least 80% sequence identity thereto, a HC CDR2 comprising SEQ ID No: 446 or a sequence with at least 80% sequence identity thereto, a HC CDR3 comprising SEQ ID No: 447 or a sequence with at least 80% sequence identity thereto, a LC CDR1 comprising SEQ ID No: 448 or a sequence with at least 80% sequence identity thereto, a LC CDR2 comprising SEQ ID No: 449 or a sequence with at least 80% sequence identity thereto and a LC CDR3 comprising SEQ ID No: 440 or a sequence with at least 80% sequence identity thereto;
An antibody comprising a HC CDR1 comprising SEQ ID No: 455 or a sequence with at least 80% sequence identity thereto, a HC CDR2 comprising SEQ ID No: 456 or a sequence with at least 80% sequence identity thereto, a HC CDR3 comprising SEQ ID No: 457 or a sequence with at least 80% sequence identity thereto, a LC CDR1 comprising SEQ ID No: 458 or a sequence with at least 80% sequence identity thereto, a LC CDR2 comprising SEQ ID No: 459 or a sequence with at least 80% sequence identity thereto and a LC CDR3 comprising SEQ ID No: 460 or a sequence with at least 80% sequence identity thereto;
An antibody comprising a HC CDR1 comprising SEQ ID No: 465 or a sequence with at least 80% sequence identity thereto, a HC CDR2 comprising SEQ ID No: 466 or a sequence with at least 80% sequence identity thereto, a HC CDR3 comprising SEQ ID No: 467 or a sequence with at least 80% sequence identity thereto, a LC CDR1 comprising SEQ ID No: 468 or a sequence with at least 80% sequence identity thereto, a LC CDR2 comprising SEQ ID No: 469 or a sequence with at least 80% sequence identity thereto and a LC CDR3 comprising SEQ ID No: 470 or a sequence with at least 80% sequence identity thereto;
An antibody comprising a HC CDR1 comprising SEQ ID No: 475 or a sequence with at least 80% sequence identity thereto, a HC CDR2 comprising SEQ ID No: 476 or a sequence with at least 80% sequence identity thereto, a HC CDR3 comprising SEQ ID No: 477 or a sequence with at least 80% sequence identity thereto, a LC CDR1 comprising SEQ ID No: 478 or a sequence with at least 80% sequence identity thereto, a LC CDR2 comprising SEQ ID No: 479 or a sequence with at least 80% sequence identity thereto and a LC CDR3 comprising SEQ ID No: 480 or a sequence with at least 80% sequence identity thereto;
An antibody comprising a HC CDR1 comprising SEQ ID No: 485 or a sequence with at least 80% sequence identity thereto, a HC CDR2 comprising SEQ ID No: 486 or a sequence with at least 80% sequence identity thereto, a HC CDR3 comprising SEQ ID No: 487 or a sequence with at least 80% sequence identity thereto, a LC CDR1 comprising SEQ ID No: 488 or a sequence with at least 80% sequence identity thereto, a LC CDR2 comprising SEQ ID No: 489 or a sequence with at least 80% sequence identity thereto and a LC CDR3 comprising SEQ ID No: 490 or a sequence with at least 80% sequence identity thereto;
An antibody comprising a HC CDR1 comprising SEQ ID No: 495 or a sequence with at least 80% sequence identity thereto, a HC CDR2 comprising SEQ ID No: 496 or a sequence with at least 80% sequence identity thereto, a HC CDR3 comprising SEQ ID No: 497 or a sequence with at least 80% sequence identity thereto, a LC CDR1 comprising SEQ ID No: 498 or a sequence with at least 80% sequence identity thereto, a LC CDR2 comprising SEQ ID No: 499 or a sequence with at least 80% sequence identity thereto and a LC CDR3 comprising SEQ ID No: 500 or a sequence with at least 80% sequence identity thereto;
An antibody comprising a HC CDR1 comprising SEQ ID No: 505 or a sequence with at least 80% sequence identity thereto, a HC CDR2 comprising SEQ ID No: 506 or a sequence with at least 80% sequence identity thereto, a HC CDR3 comprising SEQ ID No: 507 or a sequence with at least 80% sequence identity thereto, a LC CDR1 comprising SEQ ID No: 508 or a sequence with at least 80% sequence identity thereto, a LC CDR2 comprising SEQ ID No: 509 or a sequence with at least 80% sequence identity thereto and a LC CDR3 comprising SEQ ID No: 510 or a sequence with at least 80% sequence identity thereto;
An antibody comprising a HC CDR1 comprising SEQ ID No: 515 or a sequence with at least 80% sequence identity thereto, a HC CDR2 comprising SEQ ID No: 516 or a sequence with at least 80% sequence identity thereto, a HC CDR3 comprising SEQ ID No: 517 or a sequence with at least 80% sequence identity thereto, a LC CDR1 comprising SEQ ID No: 518 or a sequence with at least 80% sequence identity thereto, a LC CDR2 comprising SEQ ID No: 519 or a sequence with at least 80% sequence identity thereto and a LC CDR3 comprising SEQ ID No: 520 or a sequence with at least 80% sequence identity thereto;
An antibody comprising a HC CDR1 comprising SEQ ID No: 525 or a sequence with at least 80% sequence identity thereto, a HC CDR2 comprising SEQ ID No: 526 or a sequence with at least 80% sequence identity thereto, a HC CDR3 comprising SEQ ID No: 527 or a sequence with at least 80% sequence identity thereto, a LC CDR1 comprising SEQ ID No: 528 or a sequence with at least 80% sequence identity thereto, a LC CDR2 comprising SEQ ID No: 529 or a sequence with at least 80% sequence identity thereto and a LC CDR3 comprising SEQ ID No: 530 or a sequence with at least 80% sequence identity thereto;
An antibody comprising a HC CDR1 comprising SEQ ID No: 535 or a sequence with at least 80% sequence identity thereto, a HC CDR2 comprising SEQ ID No: 536 or a sequence with at least 80% sequence identity thereto, a HC CDR3 comprising SEQ ID No: 537 or a sequence with at least 80% sequence identity thereto, a LC CDR1 comprising SEQ ID No: 538 or a sequence with at least 80% sequence identity thereto, a LC CDR2 comprising SEQ ID No: 539 or a sequence with at least 80% sequence identity thereto and a LC CDR3 comprising SEQ ID No: 540 or a sequence with at least 80% sequence identity thereto;
An antibody comprising a HC CDR1 comprising SEQ ID No: 545 or a sequence with at least 80% sequence identity thereto, a HC CDR2 comprising SEQ ID No: 546 or a sequence with at least 80% sequence identity thereto, a HC CDR3 comprising SEQ ID No: 547 or a sequence with at least 80% sequence identity thereto, a LC CDR1 comprising SEQ ID No: 548 or a sequence with at least 80% sequence identity thereto, a LC CDR2 comprising SEQ ID No: 549 or a sequence with at least 80% sequence identity thereto and a LC CDR3 comprising SEQ ID No: 550 or a sequence with at least 80% sequence identity thereto;
An antibody comprising a HC CDR1 comprising SEQ ID No: 555 or a sequence with at least 80% sequence identity thereto, a HC CDR2 comprising SEQ ID No: 556 or a sequence with at least 80% sequence identity thereto, a HC CDR3 comprising SEQ ID No: 557 or a sequence with at least 80% sequence identity thereto, a LC CDR1 comprising SEQ ID No: 558 or a sequence with at least 80% sequence identity thereto, a LC CDR2 comprising SEQ ID No: 559 or a sequence with at least 80% sequence identity thereto and a LC CDR3 comprising SEQ ID No: 560 or a sequence with at least 80% sequence identity thereto;
An antibody comprising a HC CDR1 comprising SEQ ID No: 565 or a sequence with at least 80% sequence identity thereto, a HC CDR2 comprising SEQ ID No: 566 or a sequence with at least 80% sequence identity thereto, a HC CDR3 comprising SEQ ID No: 567 or a sequence with at least 80% sequence identity thereto, a LC CDR1 comprising SEQ ID No: 568 or a sequence with at least 80% sequence identity thereto, a LC CDR2 comprising SEQ ID No: 569 or a sequence with at least 80% sequence identity thereto and a LC CDR3 comprising SEQ ID No: 570 or a sequence with at least 80% sequence identity thereto;
An antibody comprising a HC CDR1 comprising SEQ ID No: 575 or a sequence with at least 80% sequence identity thereto, a HC CDR2 comprising SEQ ID No: 576 or a sequence with at least 80% sequence identity thereto, a HC CDR3 comprising SEQ ID No: 577 or a sequence with at least 80% sequence identity thereto, a LC CDR1 comprising SEQ ID No: 578 or a sequence with at least 80% sequence identity thereto, a LC CDR2 comprising SEQ ID No: 579 or a sequence with at least 80% sequence identity thereto and a LC CDR3 comprising SEQ ID No: 580 or a sequence with at least 80% sequence identity thereto;
An antibody comprising a HC CDR1 comprising SEQ ID No: 585 or a sequence with at least 80% sequence identity thereto, a HC CDR2 comprising SEQ ID No: 586 or a sequence with at least 80% sequence identity thereto, a HC CDR3 comprising SEQ ID No: 587 or a sequence with at least 80% sequence identity thereto, a LC CDR1 comprising SEQ ID No: 588 or a sequence with at least 80% sequence identity thereto, a LC CDR2 comprising SEQ ID No: 589 or a sequence with at least 80% sequence identity thereto and a LC CDR3 comprising SEQ ID No: 590 or a sequence with at least 80% sequence identity thereto;
An antibody comprising a HC CDR1 comprising SEQ ID No: 595 or a sequence with at least 80% sequence identity thereto, a HC CDR2 comprising SEQ ID No: 596 or a sequence with at least 80% sequence identity thereto, a HC CDR3 comprising SEQ ID No: 597 or a sequence with at least 80% sequence identity thereto, a LC CDR1 comprising SEQ ID No: 598 or a sequence with at least 80% sequence identity thereto, a LC CDR2 comprising SEQ ID No: 599 or a sequence with at least 80% sequence identity thereto and a LC CDR3 comprising SEQ ID No: 600 or a sequence with at least 80% sequence identity thereto;
An antibody comprising a HC CDR1 comprising SEQ ID No: 605 or a sequence with at least 80% sequence identity thereto, a HC CDR2 comprising SEQ ID No: 606 or a sequence with at least 80% sequence identity thereto, a HC CDR3 comprising SEQ ID No: 607 or a sequence with at least 80% sequence identity thereto, a LC CDR1 comprising SEQ ID No: 608 or a sequence with at least 80% sequence identity thereto, a LC CDR2 comprising SEQ ID No: 609 or a sequence with at least 80% sequence identity thereto and a LC CDR3 comprising SEQ ID No: 610 or a sequence with at least 80% sequence identity thereto;
An antibody comprising a HC CDR1 comprising SEQ ID No: 615 or a sequence with at least 80% sequence identity thereto, a HC CDR2 comprising SEQ ID No: 616 or a sequence with at least 80% sequence identity thereto, a HC CDR3 comprising SEQ ID No: 617 or a sequence with at least 80% sequence identity thereto, a LC CDR1 comprising SEQ ID No: 618 or a sequence with at least 80% sequence identity thereto, a LC CDR2 comprising SEQ ID No: 619 or a sequence with at least 80% sequence identity thereto and a LC CDR3 comprising SEQ ID No: 620 or a sequence with at least 80% sequence identity thereto;
An antibody comprising a HC CDR1 comprising SEQ ID No: 625 or a sequence with at least 80% sequence identity thereto, a HC CDR2 comprising SEQ ID No: 626 or a sequence with at least 80% sequence identity thereto, a HC CDR3 comprising SEQ ID No: 627 or a sequence with at least 80% sequence identity thereto, a LC CDR1 comprising SEQ ID No: 628 or a sequence with at least 80% sequence identity thereto, a LC CDR2 comprising SEQ ID No: 629 or a sequence with at least 80% sequence identity thereto and a LC CDR3 comprising SEQ ID No: 630 or a sequence with at least 80% sequence identity thereto;
An antibody comprising a HC CDR1 comprising SEQ ID No: 635 or a sequence with at least 80% sequence identity thereto, a HC CDR2 comprising SEQ ID No: 636 or a sequence with at least 80% sequence identity thereto, a HC CDR3 comprising SEQ ID No: 637 or a sequence with at least 80% sequence identity thereto, a LC CDR1 comprising SEQ ID No: 638 or a sequence with at least 80% sequence identity thereto, a LC CDR2 comprising SEQ ID No: 639 or a sequence with at least 80% sequence identity thereto and a LC CDR3 comprising SEQ ID No: 640 or a sequence with at least 80% sequence identity thereto;
An antibody comprising a HC CDR1 comprising SEQ ID No: 645 or a sequence with at least 80% sequence identity thereto, a HC CDR2 comprising SEQ ID No: 646 or a sequence with at least 80% sequence identity thereto, a HC CDR3 comprising SEQ ID No: 647 or a sequence with at least 80% sequence identity thereto, a LC CDR1 comprising SEQ ID No: 648 or a sequence with at least 80% sequence identity thereto, a LC CDR2 comprising SEQ ID No: 649 or a sequence with at least 80% sequence identity thereto and a LC CDR3 comprising SEQ ID No: 650 or a sequence with at least 80% sequence identity thereto;
An antibody comprising a HC CDR1 comprising SEQ ID No: 655 or a sequence with at least 80% sequence identity thereto, a HC CDR2 comprising SEQ ID No: 656 or a sequence with at least 80% sequence identity thereto, a HC CDR3 comprising SEQ ID No: 657 or a sequence with at least 80% sequence identity thereto, a LC CDR1 comprising SEQ ID No: 658 or a sequence with at least 80% sequence identity thereto, a LC CDR2 comprising SEQ ID No: 659 or a sequence with at least 80% sequence identity thereto and a LC CDR3 comprising SEQ ID No: 660 or a sequence with at least 80% sequence identity thereto;
An antibody comprising a HC CDR1 comprising SEQ ID No: 665 or a sequence with at least 80% sequence identity thereto, a HC CDR2 comprising SEQ ID No: 666 or a sequence with at least 80% sequence identity thereto, a HC CDR3 comprising SEQ ID No: 667 or a sequence with at least 80% sequence identity thereto, a LC CDR1 comprising SEQ ID No: 668 or a sequence with at least 80% sequence identity thereto, a LC CDR2 comprising SEQ ID No: 669 or a sequence with at least 80% sequence identity thereto and a LC CDR3 comprising SEQ ID No: 670 or a sequence with at least 80% sequence identity thereto;
An antibody comprising a HC CDR1 comprising SEQ ID No: 675 or a sequence with at least 80% sequence identity thereto, a HC CDR2 comprising SEQ ID No: 676 or a sequence with at least 80% sequence identity thereto, a HC CDR3 comprising SEQ ID No: 677 or a sequence with at least 80% sequence identity thereto, a LC CDR1 comprising SEQ ID No: 678 or a sequence with at least 80% sequence identity thereto, a LC CDR2 comprising SEQ ID No: 679 or a sequence with at least 80% sequence identity thereto and a LC CDR3 comprising SEQ ID No: 680 or a sequence with at least 80% sequence identity thereto;
An antibody comprising a HC CDR1 comprising SEQ ID No: 685 or a sequence with at least 80% sequence identity thereto, a HC CDR2 comprising SEQ ID No: 686 or a sequence with at least 80% sequence identity thereto, a HC CDR3 comprising SEQ ID No: 687 or a sequence with at least 80% sequence identity thereto, a LC CDR1 comprising SEQ ID No: 688 or a sequence with at least 80% sequence identity thereto, a LC CDR2 comprising SEQ ID No: 689 or a sequence with at least 80% sequence identity thereto and a LC CDR3 comprising SEQ ID No: 690 or a sequence with at least 80% sequence identity thereto;
An antibody comprising a HC CDR1 comprising SEQ ID No: 695 or a sequence with at least 80% sequence identity thereto, a HC CDR2 comprising SEQ ID No: 696 or a sequence with at least 80% sequence identity thereto, a HC CDR3 comprising SEQ ID No: 697 or a sequence with at least 80% sequence identity thereto, a LC CDR1 comprising SEQ ID No: 698 or a sequence with at least 80% sequence identity thereto, a LC CDR2 comprising SEQ ID No: 699 or a sequence with at least 80% sequence identity thereto and a LC CDR3 comprising SEQ ID No: 700 or a sequence with at least 80% sequence identity thereto;
An antibody comprising a HC CDR1 comprising SEQ ID No: 705 or a sequence with at least 80% sequence identity thereto, a HC CDR2 comprising SEQ ID No: 706 or a sequence with at least 80% sequence identity thereto, a HC CDR3 comprising SEQ ID No: 707 or a sequence with at least 80% sequence identity thereto, a LC CDR1 comprising SEQ ID No: 708 or a sequence with at least 80% sequence identity thereto, a LC CDR2 comprising SEQ ID No: 709 or a sequence with at least 80% sequence identity thereto and a LC CDR3 comprising SEQ ID No: 710 or a sequence with at least 80% sequence identity thereto; or
An antibody comprising a HC CDR1 comprising SEQ ID No: 715 or a sequence with at least 80% sequence identity thereto, a HC CDR2 comprising SEQ ID No: 716 or a sequence with at least 80% sequence identity thereto, a HC CDR3 comprising SEQ ID No: 717 or a sequence with at least 80% sequence identity thereto, a LC CDR1 comprising SEQ ID No: 718 or a sequence with at least 80% sequence identity thereto, a LC CDR2 comprising SEQ ID No: 719 or a sequence with at least 80% sequence identity thereto and a LC CDR3 comprising SEQ ID No: 720 or a sequence with at least 80% sequence identity thereto.
An antibody comprising a HC CDR1 comprising SEQ ID No: 741 or a sequence with at least 80% sequence identity thereto, a HC CDR2 comprising SEQ ID No: 742 or a sequence with at least 80% sequence identity thereto, a HC CDR3 comprising SEQ ID No: 743 or a sequence with at least 80% sequence identity thereto, a LC CDR1 comprising SEQ ID No: 744 or a sequence with at least 80% sequence identity thereto, a LC CDR2 comprising SEQ ID No: 745 or a sequence with at least 80% sequence identity thereto and a LC CDR3 comprising SEQ ID No: 746 or a sequence with at least 80% sequence identity thereto.
An antibody comprising a HC CDR1 comprising SEQ ID No: 751 or a sequence with at least 80% sequence identity thereto, a HC CDR2 comprising SEQ ID No: 752 or a sequence with at least 80% sequence identity thereto, a HC CDR3 comprising SEQ ID No: 753 or a sequence with at least 80% sequence identity thereto, a LC CDR1 comprising SEQ ID No: 754 or a sequence with at least 80% sequence identity thereto, a LC CDR2 comprising SEQ ID No: 755 or a sequence with at least 80% sequence identity thereto and a LC CDR3 comprising SEQ ID No: 756 or a sequence with at least 80% sequence identity thereto.
An antibody comprising a HC CDR1 comprising SEQ ID No: 761 or a sequence with at least 80% sequence identity thereto, a HC CDR2 comprising SEQ ID No: 762 or a sequence with at least 80% sequence identity thereto, a HC CDR3 comprising SEQ ID No: 763 or a sequence with at least 80% sequence identity thereto, a LC CDR1 comprising SEQ ID No: 764 or a sequence with at least 80% sequence identity thereto, a LC CDR2 comprising SEQ ID No: 765 or a sequence with at least 80% sequence identity thereto and a LC CDR3 comprising SEQ ID No: 766 or a sequence with at least 80% sequence identity thereto.
An antibody comprising a HC CDR1 comprising SEQ ID No: 771 or a sequence with at least 80% sequence identity thereto, a HC CDR2 comprising SEQ ID No: 772 or a sequence with at least 80% sequence identity thereto, a HC CDR3 comprising SEQ ID No: 773 or a sequence with at least 80% sequence identity thereto, a LC CDR1 comprising SEQ ID No: 774 or a sequence with at least 80% sequence identity thereto, a LC CDR2 comprising SEQ ID No: 775 or a sequence with at least 80% sequence identity thereto and a LC CDR3 comprising SEQ ID No: 776 or a sequence with at least 80% sequence identity thereto.
An antibody comprising a HC CDR1 comprising SEQ ID No: 781 or a sequence with at least 80% sequence identity thereto, a HC CDR2 comprising SEQ ID No: 782 or a sequence with at least 80% sequence identity thereto, a HC CDR3 comprising SEQ ID No: 783 or a sequence with at least 80% sequence identity thereto, a LC CDR1 comprising SEQ ID No: 784 or a sequence with at least 80% sequence identity thereto, a LC CDR2 comprising SEQ ID No: 785 or a sequence with at least 80% sequence identity thereto and a LC CDR3 comprising SEQ ID No: 786 or a sequence with at least 80% sequence identity thereto.
An antibody comprising a HC CDR1 comprising SEQ ID No: 791 or a sequence with at least 80% sequence identity thereto, a HC CDR2 comprising SEQ ID No: 792 or a sequence with at least 80% sequence identity thereto, a HC CDR3 comprising SEQ ID No: 793 or a sequence with at least 80% sequence identity thereto, a LC CDR1 comprising SEQ ID No: 794 or a sequence with at least 80% sequence identity thereto, a LC CDR2 comprising SEQ ID No: 795 or a sequence with at least 80% sequence identity thereto and a LC CDR3 comprising SEQ ID No: 796 or a sequence with at least 80% sequence identity thereto.
An antibody comprising a HC CDR1 comprising SEQ ID No: 801 or a sequence with at least 80% sequence identity thereto, a HC CDR2 comprising SEQ ID No: 802 or a sequence with at least 80% sequence identity thereto, a HC CDR3 comprising SEQ ID No: 803 or a sequence with at least 80% sequence identity thereto, a LC CDR1 comprising SEQ ID No: 804 or a sequence with at least 80% sequence identity thereto, a LC CDR2 comprising SEQ ID No: 805 or a sequence with at least 80% sequence identity thereto and a LC CDR3 comprising SEQ ID No: 806 or a sequence with at least 80% sequence identity thereto.

In an embodiment the antibody is selected from one of those recited above but comprises a sequence identity of 85%, 90%, 95%, 96%, 97%, 98% or 99% identity to the HC and/or LC CDR1, 2 and/or CDR3 sequences that are listed.
An antibody comprising a HC CDR1 comprising SEQ ID No: 741 or a sequence with at least 60%, 70%, 80%, 90% or 95% sequence identity thereto, a HC CDR2 comprising SEQ ID No: 742 or a sequence with at least 60%, 70%, 80%, 90% or 95% sequence identity thereto, a HC CDR3 comprising SEQ ID No: 743 or a sequence with at least 60%, 70%, 80%, 90% or 95% sequence identity thereto, a LC CDR1 comprising SEQ ID No: 744 or a sequence with at least 60%, 70%, 80%, 90% or 95% sequence identity thereto, a LC CDR2 comprising SEQ ID No: 745 or a sequence with at least 60%, 70%, 80%, 90% or 95% sequence identity thereto and a LC CDR3 comprising SEQ ID No: 746 or a sequence with at least 60%, 70%, 80%, 90% or 95% sequence identity thereto.
An antibody comprising a HC CDR1 comprising SEQ ID No: 751 or a sequence with at least 60%, 70%, 80%, 90% or 95% sequence identity thereto, a HC CDR2 comprising SEQ ID No: 752 or a sequence with at least 60%, 70%, 80%, 90% or 95% sequence identity thereto, a HC CDR3 comprising SEQ ID No: 753 or a sequence with at least 60%, 70%, 80%, 90% or 95% sequence identity thereto, a LC CDR1 comprising SEQ ID No: 754 or a sequence with at least 60%, 70%, 80%, 90% or 95% sequence identity thereto, a LC CDR2 comprising SEQ ID No: 755 or a sequence with at least 60%, 70%, 80%, 90% or 95% sequence identity thereto and a LC CDR3 comprising SEQ ID No: 756 or a sequence with at least 60%, 70%, 80%, 90% or 95% sequence identity thereto.
An antibody comprising a HC CDR1 comprising SEQ ID No: 761 or a sequence with at least 60%, 70%, 80%, 90% or 95% sequence identity thereto, a HC CDR2 comprising SEQ ID No: 762 or a sequence with at least 60%, 70%, 80%, 90% or 95% sequence identity thereto, a HC CDR3 comprising SEQ ID No: 763 or a sequence with at least 60%, 70%, 80%, 90% or 95% sequence identity thereto, a LC CDR1 comprising SEQ ID No: 764 or a sequence with at least 60%, 70%, 80%, 90% or 95% sequence identity thereto, a LC CDR2 comprising SEQ ID No: 765 or a sequence with at least 60%, 70%, 80%, 90% or 95% sequence identity thereto and a LC CDR3 comprising SEQ ID No: 766 or a sequence with at least 60%, 70%, 80%, 90% or 95% sequence identity thereto.
An antibody comprising a HC CDR1 comprising SEQ ID No: 771 or a sequence with at least 60%, 70%, 80%, 90% or 95% sequence identity thereto, a HC CDR2 comprising SEQ ID No: 772 or a sequence with at least 60%, 70%, 80%, 90% or 95% sequence identity thereto, a HC CDR3 comprising SEQ ID No: 773 or a sequence with at least 60%, 70%, 80%, 90% or 95% sequence identity thereto, a LC CDR1 comprising SEQ ID No: 774 or a sequence with at least 60%, 70%, 80%, 90% or 95% sequence identity thereto, a LC CDR2 comprising SEQ ID No: 775 or a sequence with at least 60%, 70%, 80%, 90% or 95% sequence identity thereto and a LC CDR3 comprising SEQ ID No: 776 or a sequence with at least 60%, 70%, 80%, 90% or 95% sequence identity thereto.
An antibody comprising a HC CDR1 comprising SEQ ID No: 781 or a sequence with at least 60%, 70%, 80%, 90% or 95% sequence identity thereto, a HC CDR2 comprising SEQ ID No: 782 or a sequence with at least 60%, 70%, 80%, 90% or 95% sequence identity thereto, a HC CDR3 comprising SEQ ID No: 783 or a sequence with at least 60%, 70%, 80%, 90% or 95% sequence identity thereto, a LC CDR1 comprising SEQ ID No: 784 or a sequence with at least 60%, 70%, 80%, 90% or 95% sequence identity thereto, a LC CDR2 comprising SEQ ID No: 785 or a sequence with at least 60%, 70%, 80%, 90% or 95% sequence identity thereto and a LC CDR3 comprising SEQ ID No: 786 or a sequence with at least 60%, 70%, 80%, 90% or 95% sequence identity thereto.
An antibody comprising a HC CDR1 comprising SEQ ID No: 791 or a sequence with at least 60%, 70%, 80%, 90% or 95% sequence identity thereto, a HC CDR2 comprising SEQ ID No: 792 or a sequence with at least 60%, 70%, 80%, 90% or 95% sequence identity thereto, a HC CDR3 comprising SEQ ID No: 793 or a sequence with at least 60%, 70%, 80%, 90% or 95% sequence identity thereto, a LC CDR1 comprising SEQ ID No: 794 or a sequence with at least 60%, 70%, 80%, 90% or 95% sequence identity thereto, a LC CDR2 comprising SEQ ID No: 795 or a sequence with at least 60%, 70%, 80%, 90% or 95% sequence identity thereto and a LC CDR3 comprising SEQ ID No: 796 or a sequence with at least 60%, 70%, 80%, 90% or 95% sequence identity thereto.
An antibody comprising a HC CDR1 comprising SEQ ID No: 801 or a sequence with at least 60%, 70%, 80%, 90% or 95% sequence identity thereto, a HC CDR2 comprising SEQ ID No: 802 or a sequence with at least 60%, 70%, 80%, 90% or 95% sequence identity thereto, a HC CDR3 comprising SEQ ID No: 803 or a sequence with at least 60%, 70%, 80%, 90% or 95% sequence identity thereto, a LC CDR1 comprising SEQ ID No: 804 or a sequence with at least 60%, 70%, 80%, 90% or 95% sequence identity thereto, a LC CDR2 comprising SEQ ID No: 805 or a sequence with at least 60%, 70%, 80%, 90% or 95% sequence identity thereto and a LC CDR3 comprising SEQ ID No: 806 or a sequence with at least 60%, 70%, 80%, 90% or 95% sequence identity thereto.

In an embodiment the antibody is selected from one of those recited above but comprises a sequence identity of 85%, 90%, 95%, 96%, 97%, 98% or 99% identity to the HC and/or LC CDR1, 2 and/or CDR3 sequences that are listed.

In one embodiment the antibody is selected from one of those recited above, but comprises one or more HC and/or LC CDR1, 2 and/or CDR3 sequences with 1, 2, 3, 4 or 5 amino acid substitutions compared to the sequences defined in the SEQ ID Nos. above.

In a preferred embodiment the antibody comprises a HC CDR1 comprising SEQ ID No: 575 or a sequence with at least 60%, 70%, 80%, 90% or 95% sequence identity thereto, a HC CDR2 comprising SEQ ID No: 576 or a sequence with at least 60%, 70%, 80%, 90% or 95% sequence identity thereto, a HC CDR3 comprising SEQ ID No: 577 or a sequence with at least 60%, 70%, 80%, 90% or 95% sequence identity thereto, a LC CDR1 comprising SEQ ID No: 578 or a sequence with at least 60%, 70%, 80%, 90% or 95% sequence identity thereto, a LC CDR2 comprising SEQ ID No: 579 or a sequence with at least 60%, 70%, 80%, 90% or 95% sequence identity thereto and a LC CDR3 comprising SEQ ID No: 580 or a sequence with at least 60%, 70%, 80%, 90% or 95% sequence identity thereto; or
a HC CDR1 comprising SEQ ID No: 741 or a sequence with at least 60%, 70%, 80%, 90% or 95% sequence identity thereto, a HC CDR2 comprising SEQ ID No: 742 or a sequence with at least 60%, 70%, 80%, 90% or 95% sequence identity thereto, a HC CDR3 comprising SEQ ID No: 743 or a sequence with at least 60%, 70%, 80%, 90% or 95% sequence identity thereto, a LC CDR1 comprising SEQ ID No: 744 or a sequence with at least 60%, 70%, 80%, 90% or 95% sequence identity thereto, a LC CDR2 comprising SEQ ID No: 745 or a sequence with at least 60%, 70%, 80%, 90% or 95% sequence identity thereto and a LC CDR3 comprising SEQ ID No: 746 or a sequence with at least 60%, 70%, 80%, 90% or 95% sequence identity thereto; or
a HC CDR1 comprising SEQ ID No: 275 or a sequence with at least 60%, 70%, 80%, 90% or 95% sequence identity thereto, a HC CDR2 comprising SEQ ID No: 276 or a sequence with at least 60%, 70%, 80%, 90% or 95% sequence identity thereto, a HC CDR3 comprising SEQ ID No: 277 or a sequence with at least 60%, 70%, 80%, 90% or 95% sequence identity thereto, a LC CDR1 comprising SEQ ID No: 278 or a sequence with at least 60%, 70%, 80%, 90% or 95% sequence identity thereto, a LC CDR2 comprising SEQ ID No: 279 or a sequence with at least 60%, 70%, 80%, 90% or 95% sequence identity thereto and a LC CDR3 comprising SEQ ID No: 280 or a sequence with at least 60%, 70%, 80%, 90% or 95% sequence identity thereto; or
a HC CDR1 comprising SEQ ID No: 395 or a sequence with at least 60%, 70%, 80%, 90% or 95% sequence identity thereto, a HC CDR2 comprising SEQ ID No: 396 or a sequence with at least 60%, 70%, 80%, 90% or 95% sequence identity thereto, a HC CDR3 comprising SEQ ID No: 397 or a sequence with at least 60%, 70%, 80%, 90% or 95% sequence identity thereto, a LC CDR1 comprising SEQ ID No: 398 or a sequence with at least 60%, 70%, 80%, 90% or 95% sequence identity thereto, a LC CDR2 comprising SEQ ID No: 399 or a sequence with at least 60%, 70%, 80%, 90% or 95% sequence identity thereto and a LC CDR3 comprising SEQ ID No: 400 or a sequence with at least 60%, 70%, 80%, 90% or 95% sequence identity thereto.

Fragments of the antibodies listed above are also provided and within the scope of the invention.

In one embodiment, the antibody may be selected from one of the following antibodies:
An antibody comprising a HC variable region comprising or consisting of SEQ ID No. 12 or a sequence with at least 80% sequence identity thereto and a LC variable region comprising or consisting of SEQ ID No. 14 or a sequence with at least 80% sequence identity thereto;
An antibody comprising a HC variable region comprising or consisting of SEQ ID No. 22 or a sequence with at least 80% sequence identity thereto and a LC variable region comprising or consisting of SEQ ID No. 24 or a sequence with at least 80% sequence identity thereto;
An antibody comprising a HC variable region comprising or consisting of SEQ ID No. 32 or a sequence with at least 80% sequence identity thereto and a LC variable region comprising or consisting of SEQ ID No. 34 or a sequence with at least 80% sequence identity thereto;
An antibody comprising a HC variable region comprising or consisting of SEQ ID No. 42 or a sequence with at least 80% sequence identity thereto and a LC variable region comprising or consisting of SEQ ID No. 44 or a sequence with at least 80% sequence identity thereto;
An antibody comprising a HC variable region comprising or consisting of SEQ ID No. 52 or a sequence with at least 80% sequence identity thereto and a LC variable region comprising or consisting of SEQ ID No. 54 or a sequence with at least 80% sequence identity thereto;
An antibody comprising a HC variable region comprising or consisting of SEQ ID No. 62 or a sequence with at least 80% sequence identity thereto and a LC variable region comprising or consisting of SEQ ID No. 64 or a sequence with at least 80% sequence identity thereto;
An antibody comprising a HC variable region comprising or consisting of SEQ ID No. 72 or a sequence with at least 80% sequence identity thereto and a LC variable region comprising or consisting of SEQ ID No. 74 or a sequence with at least 80% sequence identity thereto;
An antibody comprising a HC variable region comprising or consisting of SEQ ID No. 82 or a sequence with at least 80% sequence identity thereto and a LC variable region comprising or consisting of SEQ ID No. 84 or a sequence with at least 80% sequence identity thereto;
An antibody comprising a HC variable region comprising or consisting of SEQ ID No. 92 or a sequence with at least 80% sequence identity thereto and a LC variable region comprising or consisting of SEQ ID No. 94 or a sequence with at least 80% sequence identity thereto;
An antibody comprising a HC variable region comprising or consisting of SEQ ID No. 102 or a sequence with at least 80% sequence identity thereto and a LC variable region comprising or consisting of SEQ ID No. 104 or a sequence with at least 80% sequence identity thereto;
An antibody comprising a HC variable region comprising or consisting of SEQ ID No. 112 or a sequence with at least 80% sequence identity thereto and a LC variable region comprising or consisting of SEQ ID No. 114 or a sequence with at least 80% sequence identity thereto;
An antibody comprising a HC variable region comprising or consisting of SEQ ID No. 122 or a sequence with at least 80% sequence identity thereto and a LC variable region comprising or consisting of SEQ ID No. 124 or a sequence with at least 80% sequence identity thereto;
An antibody comprising a HC variable region comprising or consisting of SEQ ID No. 132 or a sequence with at least 80% sequence identity thereto and a LC variable region comprising or consisting of SEQ ID No. 134 or a sequence with at least 80% sequence identity thereto;
An antibody comprising a HC variable region comprising or consisting of SEQ ID No. 142 or a sequence with at least 80% sequence identity thereto and a LC variable region comprising or consisting of SEQ ID No. 144 or a sequence with at least 80% sequence identity thereto;
An antibody comprising a HC variable region comprising or consisting of SEQ ID No. 152 or a sequence with at least 80% sequence identity thereto and a LC variable region comprising or consisting of SEQ ID No. 154 or a sequence with at least 80% sequence identity thereto;
An antibody comprising a HC variable region comprising or consisting of SEQ ID No. 162 or a sequence with at least 80% sequence identity thereto and a LC variable region comprising or consisting of SEQ ID No. 164 or a sequence with at least 80% sequence identity thereto;
An antibody comprising a HC variable region comprising or consisting of SEQ ID No. 172 or a sequence with at least 80% sequence identity thereto and a LC variable region comprising or consisting of SEQ ID No. 174 or a sequence with at least 80% sequence identity thereto;
An antibody comprising a HC variable region comprising or consisting of SEQ ID No. 182 or a sequence with at least 80% sequence identity thereto and a LC variable region comprising or consisting of SEQ ID No. 184 or a sequence with at least 80% sequence identity thereto;
An antibody comprising a HC variable region comprising or consisting of SEQ ID No. 192 or a sequence with at least 80% sequence identity thereto and a LC variable region comprising or consisting of SEQ ID No. 194 or a sequence with at least 80% sequence identity thereto;
An antibody comprising a HC variable region comprising or consisting of SEQ ID No. 202 or a sequence with at least 80% sequence identity thereto and a LC variable region comprising or consisting of SEQ ID No. 204 or a sequence with at least 80% sequence identity thereto;
An antibody comprising a HC variable region comprising or consisting of SEQ ID No. 212 or a sequence with at least 80% sequence identity thereto and a LC variable region comprising or consisting of SEQ ID No. 214 or a sequence with at least 80% sequence identity thereto;
An antibody comprising a HC variable region comprising or consisting of SEQ ID No. 222 or a sequence with at least 80% sequence identity thereto and a LC variable region comprising or consisting of SEQ ID No. 224 or a sequence with at least 80% sequence identity thereto;
An antibody comprising a HC variable region comprising or consisting of SEQ ID No. 232 or a sequence with at least 80% sequence identity thereto and a LC variable region comprising or consisting of SEQ ID No. 234 or a sequence with at least 80% sequence identity thereto;
An antibody comprising a HC variable region comprising or consisting of SEQ ID No. 242 or a sequence with at least 80% sequence identity thereto and a LC variable region comprising or consisting of SEQ ID No. 244 or a sequence with at least 80% sequence identity thereto;
An antibody comprising a HC variable region comprising or consisting of SEQ ID No. 252 or a sequence with at least 80% sequence identity thereto and a LC variable region comprising or consisting of SEQ ID No. 254 or a sequence with at least 80% sequence identity thereto;
An antibody comprising a HC variable region comprising or consisting of SEQ ID No. 262 or a sequence with at least 80% sequence identity thereto and a LC variable region comprising or consisting of SEQ ID No. 264 or a sequence with at least 80% sequence identity thereto;
An antibody comprising a HC variable region comprising or consisting of SEQ ID No. 272 or a sequence with at least 80% sequence identity thereto and a LC variable region comprising or consisting of SEQ ID No. 274 or a sequence with at least 80% sequence identity thereto;
An antibody comprising a HC variable region comprising or consisting of SEQ ID No. 282 or a sequence with at least 80% sequence identity thereto and a LC variable region comprising or consisting of SEQ ID No. 284 or a sequence with at least 80% sequence identity thereto;
An antibody comprising a HC variable region comprising or consisting of SEQ ID No. 292 or a sequence with at least 80% sequence identity thereto and a LC variable region comprising or consisting of SEQ ID No. 294 or a sequence with at least 80% sequence identity thereto;
An antibody comprising a HC variable region comprising or consisting of SEQ ID No. 302 or a sequence with at least 80% sequence identity thereto and a LC variable region comprising or consisting of SEQ ID No. 304 or a sequence with at least 80% sequence identity thereto;
An antibody comprising a HC variable region comprising or consisting of SEQ ID No. 312 or a sequence with at least 80% sequence identity thereto and a LC variable region comprising or consisting of SEQ ID No. 314 or a sequence with at least 80% sequence identity thereto;
An antibody comprising a HC variable region comprising or consisting of SEQ ID No. 322 or a sequence with at least 80% sequence identity thereto and a LC variable region comprising or consisting of SEQ ID No. 324 or a sequence with at least 80% sequence identity thereto;
An antibody comprising a HC variable region comprising or consisting of SEQ ID No. 332 or a sequence with at least 80% sequence identity thereto and a LC variable region comprising or consisting of SEQ ID No. 334 or a sequence with at least 80% sequence identity thereto;
An antibody comprising a HC variable region comprising or consisting of SEQ ID No. 342 or a sequence with at least 80% sequence identity thereto and a LC variable region comprising or consisting of SEQ ID No. 344 or a sequence with at least 80% sequence identity thereto;
An antibody comprising a HC variable region comprising or consisting of SEQ ID No. 352 or a sequence with at least 80% sequence identity thereto and a LC variable region comprising or consisting of SEQ ID No. 354 or a sequence with at least 80% sequence identity thereto;
An antibody comprising a HC variable region comprising or consisting of SEQ ID No. 362 or a sequence with at least 80% sequence identity thereto and a LC variable region comprising or consisting of SEQ ID No. 364 or a sequence with at least 80% sequence identity thereto;
An antibody comprising a HC variable region comprising or consisting of SEQ ID No. 372 or a sequence with at least 80% sequence identity thereto and a LC variable region comprising or consisting of SEQ ID No. 374 or a sequence with at least 80% sequence identity thereto;
An antibody comprising a HC variable region comprising or consisting of SEQ ID No. 382 or a sequence with at least 80% sequence identity thereto and a LC variable region comprising or consisting of SEQ ID No. 384 or a sequence with at least 80% sequence identity thereto;
An antibody comprising a HC variable region comprising or consisting of SEQ ID No. 392 or a sequence with at least 80% sequence identity thereto and a LC variable region comprising or consisting of SEQ ID No. 394 or a sequence with at least 80% sequence identity thereto;
An antibody comprising a HC variable region comprising or consisting of SEQ ID No. 402 or a sequence with at least 80% sequence identity thereto and a LC variable region comprising or consisting of SEQ ID No. 404 or a sequence with at least 80% sequence identity thereto;
An antibody comprising a HC variable region comprising or consisting of SEQ ID No. 412 or a sequence with at least 80% sequence identity thereto and a LC variable region comprising or consisting of SEQ ID No. 414 or a sequence with at least 80% sequence identity thereto;
An antibody comprising a HC variable region comprising or consisting of SEQ ID No. 422 or a sequence with at least 80% sequence identity thereto and a LC variable region comprising or consisting of SEQ ID No. 424 or a sequence with at least 80% sequence identity thereto;
An antibody comprising a HC variable region comprising or consisting of SEQ ID No. 432 or a sequence with at least 80% sequence identity thereto and a LC variable region comprising or consisting of SEQ ID No. 434 or a sequence with at least 80% sequence identity thereto;
An antibody comprising a HC variable region comprising or consisting of SEQ ID No. 442 or a sequence with at least 80% sequence identity thereto and a LC variable region comprising or consisting of SEQ ID No. 444 or a sequence with at least 80% sequence identity thereto;
An antibody comprising a HC variable region comprising or consisting of SEQ ID No. 452 or a sequence with at least 80% sequence identity thereto and a LC variable region comprising or consisting of SEQ ID No. 454 or a sequence with at least 80% sequence identity thereto;
An antibody comprising a HC variable region comprising or consisting of SEQ ID No. 462 or a sequence with at least 80% sequence identity thereto and a LC variable region comprising or consisting of SEQ ID No. 464 or a sequence with at least 80% sequence identity thereto;
An antibody comprising a HC variable region comprising or consisting of SEQ ID No. 472 or a sequence with at least 80% sequence identity thereto and a LC variable region comprising or consisting of SEQ ID No. 474 or a sequence with at least 80% sequence identity thereto;
An antibody comprising a HC variable region comprising or consisting of SEQ ID No. 482 or a sequence with at least 80% sequence identity thereto and a LC variable region comprising or consisting of SEQ ID No. 484 or a sequence with at least 80% sequence identity thereto;
An antibody comprising a HC variable region comprising or consisting of SEQ ID No. 492 or a sequence with at least 80% sequence identity thereto and a LC variable region comprising or consisting of SEQ ID No. 494 or a sequence with at least 80% sequence identity thereto;
An antibody comprising a HC variable region comprising or consisting of SEQ ID No. 502 or a sequence with at least 80% sequence identity thereto and a LC variable region comprising or consisting of SEQ ID No. 504 or a sequence with at least 80% sequence identity thereto;
An antibody comprising a HC variable region comprising or consisting of SEQ ID No. 512 or a sequence with at least 80% sequence identity thereto and a LC variable region comprising or consisting of SEQ ID No. 514 or a sequence with at least 80% sequence identity thereto;
An antibody comprising a HC variable region comprising or consisting of SEQ ID No. 522 or a sequence with at least 80% sequence identity thereto and a LC variable region comprising or consisting of SEQ ID No. 524 or a sequence with at least 80% sequence identity thereto;
An antibody comprising a HC variable region comprising or consisting of SEQ ID No. 532 or a sequence with at least 80% sequence identity thereto and a LC variable region comprising or consisting of SEQ ID No. 534 or a sequence with at least 80% sequence identity thereto;
An antibody comprising a HC variable region comprising or consisting of SEQ ID No. 542 or a sequence with at least 80% sequence identity thereto and a LC variable region comprising or consisting of SEQ ID No. 544 or a sequence with at least 80% sequence identity thereto;
An antibody comprising a HC variable region comprising or consisting of SEQ ID No. 552 or a sequence with at least 80% sequence identity thereto and a LC variable region comprising or consisting of SEQ ID No. 554 or a sequence with at least 80% sequence identity thereto;
An antibody comprising a HC variable region comprising or consisting of SEQ ID No. 562 or a sequence with at least 80% sequence identity thereto and a LC variable region comprising or consisting of SEQ ID No. 564 or a sequence with at least 80% sequence identity thereto;
An antibody comprising a HC variable region comprising or consisting of SEQ ID No. 572 or a sequence with at least 80% sequence identity thereto and a LC variable region comprising or consisting of SEQ ID No. 574 or a sequence with at least 80% sequence identity thereto;
An antibody comprising a HC variable region comprising or consisting of SEQ ID No. 582 or a sequence with at least 80% sequence identity thereto and a LC variable region comprising or consisting of SEQ ID No. 584 or a sequence with at least 80% sequence identity thereto;
An antibody comprising a HC variable region comprising or consisting of SEQ ID No. 592 or a sequence with at least 80% sequence identity thereto and a LC variable region comprising or consisting of SEQ ID No. 594 or a sequence with at least 80% sequence identity thereto;
An antibody comprising a HC variable region comprising or consisting of SEQ ID No. 602 or a sequence with at least 80% sequence identity thereto and a LC variable region comprising or consisting of SEQ ID No. 604 or a sequence with at least 80% sequence identity thereto;
An antibody comprising a HC variable region comprising or consisting of SEQ ID No. 612 or a sequence with at least 80% sequence identity thereto and a LC variable region comprising or consisting of SEQ ID No. 614 or a sequence with at least 80% sequence identity thereto;
An antibody comprising a HC variable region comprising or consisting of SEQ ID No. 622 or a sequence with at least 80% sequence identity thereto and a LC variable region comprising or consisting of SEQ ID No. 624 or a sequence with at least 80% sequence identity thereto;
An antibody comprising a HC variable region comprising or consisting of SEQ ID No. 632 or a sequence with at least 80% sequence identity thereto and a LC variable region comprising or consisting of SEQ ID No. 634 or a sequence with at least 80% sequence identity thereto;
An antibody comprising a HC variable region comprising or consisting of SEQ ID No. 642 or a sequence with at least 80% sequence identity thereto and a LC variable region comprising or consisting of SEQ ID No. 644 or a sequence with at least 80% sequence identity thereto;
An antibody comprising a HC variable region comprising or consisting of SEQ ID No. 652 or a sequence with at least 80% sequence identity thereto and a LC variable region comprising or consisting of SEQ ID No. 654 or a sequence with at least 80% sequence identity thereto;
An antibody comprising a HC variable region comprising or consisting of SEQ ID No. 662 or a sequence with at least 80% sequence identity thereto and a LC variable region comprising or consisting of SEQ ID No. 664 or a sequence with at least 80% sequence identity thereto;
An antibody comprising a HC variable region comprising or consisting of SEQ ID No. 672 or a sequence with at least 80% sequence identity thereto and a LC variable region comprising or consisting of SEQ ID No. 674 or a sequence with at least 80% sequence identity thereto;
An antibody comprising a HC variable region comprising or consisting of SEQ ID No. 682 or a sequence with at least 80% sequence identity thereto and a LC variable region comprising or consisting of SEQ ID No. 684 or a sequence with at least 80% sequence identity thereto;
An antibody comprising a HC variable region comprising or consisting of SEQ ID No. 692 or a sequence with at least 80% sequence identity thereto and a LC variable region comprising or consisting of SEQ ID No. 694 or a sequence with at least 80% sequence identity thereto;
An antibody comprising a HC variable region comprising or consisting of SEQ ID No. 702 or a sequence with at least 80% sequence identity thereto and a LC variable region comprising or consisting of SEQ ID No. 704 or a sequence with at least 80% sequence identity thereto; or
An antibody comprising a HC variable region comprising or consisting of SEQ ID No. 712 or a sequence with at least 80% sequence identity thereto and a LC variable region comprising or consisting of SEQ ID No. 714 or a sequence with at least 80% sequence identity thereto.
An antibody comprising a HC variable region comprising or consisting of SEQ ID No. 738 or a sequence with at least 80% sequence identity thereto and a LC variable region comprising or consisting of SEQ ID No. 740 or a sequence with at least 80% sequence identity thereto.
An antibody comprising a HC variable region comprising or consisting of SEQ ID No. 748 or a sequence with at least 80% sequence identity thereto and a LC variable region comprising or consisting of SEQ ID No. 750 or a sequence with at least 80% sequence identity thereto.
An antibody comprising a HC variable region comprising or consisting of SEQ ID No. 758 or a sequence with at least 80% sequence identity thereto and a LC variable region comprising or consisting of SEQ ID No. 760 or a sequence with at least 80% sequence identity thereto.
An antibody comprising a HC variable region comprising or consisting of SEQ ID No. 768 or a sequence with at least 80% sequence identity thereto and a LC variable region comprising or consisting of SEQ ID No. 770 or a sequence with at least 80% sequence identity thereto.
An antibody comprising a HC variable region comprising or consisting of SEQ ID No. 778 or a sequence with at least 80% sequence identity thereto and a LC variable region comprising or consisting of SEQ ID No. 780 or a sequence with at least 80% sequence identity thereto.
An antibody comprising a HC variable region comprising or consisting of SEQ ID No. 788 or a sequence with at least 80% sequence identity thereto and a LC variable region comprising or consisting of SEQ ID No. 790 or a sequence with at least 80% sequence identity thereto.
An antibody comprising a HC variable region comprising or consisting of SEQ ID No. 798 or a sequence with at least 80% sequence identity thereto and a LC variable region comprising or consisting of SEQ ID No. 800 or a sequence with at least 80% sequence identity thereto.

In an embodiment the antibody is selected from one of those recited above but comprises a sequence identity of 85%, 90%, 95%, 96%, 97%, 98% or 99% identity to the HC and/or LC variable region sequences that are listed.

In one embodiment the antibody is selected from one of those recited above and comprises HC variable region and/or LC variable region which comprises 1 to 20, e.g. 1 to 10, e.g., 2, 3, 4 or 5 amino acid substitutions compared to the sequences as defined by the SEQ ID Nos above.

In a preferred embodiment the antibody comprises a HC variable region comprising or consisting of SEQ ID No. 572 or a sequence with at least 60%, 70%, 80%, 90% or 95% sequence identity thereto and a LC variable region comprising or consisting of SEQ ID No. 574 or a sequence with at least 60%, 70%, 80%, 90% or 95% sequence identity thereto; or
a HC variable region comprising or consisting of SEQ ID No. 738 or a sequence with at least 60%, 70%, 80%, 90% or 95% sequence identity thereto and a LC variable region comprising or consisting of SEQ ID No. 740 or a sequence with at least 60%, 70%, 80%, 90% or 95% sequence identity thereto; or
a HC variable region comprising or consisting of SEQ ID No. 272 or a sequence with at least 60%, 70%, 80%, 90% or 95% sequence identity thereto and a LC variable region comprising or consisting of SEQ ID No. 274 or a sequence with at least 60%, 70%, 80%, 90% or 95% sequence identity thereto; or
a HC variable region comprising or consisting of SEQ ID No. 392 or a sequence with at least 60%, 70%, 80%, 90% or 95% sequence identity thereto and a LC variable region comprising or consisting of SEQ ID No. 394 or a sequence with at least 60%, 70%, 80%, 90% or 95% sequence identity thereto.

Fragments of the antibodies listed above are also provided.

The amino acid sequences of the VH and VL regions of the antibodies are shown in Table 2. An antibody or fragment may therefore be selected from an antibody comprising or consisting of a sequence shown in Table 2. In one embodiment, the antibody is selected from PMX025, PMX026, PMX027, PMX028, PMX029, PMX030, PMX031, PMX032, PMX033, PMX034, PMX035, PMX036, PMX037, PMX038, PMX039, PMX040, PMX041, PMX042, PMX043, PMX044, PMX045, PMX046, PMX047, PMX048, PMX049, PMX050, PMX051, PMX052, PMX053, PMX054, PMX055, PMX056, PMX057, PMX058, PMX059, PMX060, PMX061, PMX062, PMX063, PMX064, PMX065, PMX082, PMX083, PMX084, PMX085, PMX086, PMX087, PMX088, PMX089, PMX090, PMX091, PMX092, PMX093, PMX094, PMX095, PMX096, PMX097, PMX098, PMX099, PMX100, PMX101, PMX102, PMX103, PMX104, PMX105, PMX106, PMX107, PMX108, PMX109, PMX110, PMX111 PMX154, PMX155, PMX 156, PMX291, PMX292, PMX293, or PMX294 as set out in Table 2. In one embodiment, the antibody is PMX051 or PMX063. Sequences are also shown in Table 2. In one embodiment, the antibody is selected from PMX051, PMX63, PMX97 or PMX154.

All sequences for antibody and antibody fragments designated as PMX as shown in the figures herein and in Table 2 are within the scope of the invention.

Sequence identity as described in the various embodiments above is at least 80%. In one embodiment, sequence identity is at least 79%, 80%, 81%, 82%, 83%, 84%, 85%, 86%, 87%, 88%, 89%, 90%, 91%, 92%, 93%, 94%, 95%, 96%, 97%, 98% or 99%. In one embodiment, said sequence identity is at least 90%, 91%, 92%, 93%, 94%, 95%, 96%, 97%, 98% or 99%. The embodiments described herein also cover sequences with sequence similarity of at least 80%. In one embodiment, sequence similarity is at least 79%, 80%, 81%, 82%, 83%, 84%, 85%, 86%, 87%, 88%, 89%, 90%, 91%, 92%, 93%, 94%, 95%, 96%, 97%, 98% or 99%. In one embodiment, said sequence similarity is at least 90%, 91%, 92%, 93%, 94%, 95%, 96%, 97%, 98% or 99%.

As described above, the antibody or fragment defined by reference to the sequence may comprise one or more amino acid substitutions. In one embodiment, the modification is a conservative sequence modification. As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within one or more the CDR region and/or one or more framework region the antibody or fragment of the invention can be replaced with other amino acid residues from the same side chain family and the altered antibody can be tested for retained function (using the functional assays described herein or known in the art.

Thus, these amino acid changes can typically be made without altering the biological activity, function, or other desired property of the polypeptide, such as its affinity or its specificity for antigen. In general, single amino acid substitutions in nonessential regions of a polypeptide do not substantially alter biological activity. Furthermore, substitutions of amino acids that are similar in structure or function are less likely to disrupt the polypeptides' biological activity. Abbreviations for the amino acid residues of the polypeptides and peptides described herein, and conservative substitutions for these amino acid residues are shown in Table 1 below.

TABLE 1
Amino Acid Residues and Examples of
Conservative Amino Acid Substitutions

| Original residue | |
|---|---|
| Three letter code, single letter code | Conservative substitution |
| Alanine, Ala, A | Gly, Ser |
| Arginine, Arg, R | Lys, His |
| Asparagine, Asn, N | Gln, His |
| Aspartic acid Asp, D | Glu, Asn |
| Cysteine, Cys, C | Ser, Ala |
| Glutamine, Gln, Q | Asn |
| Glutamic acid, Glu, E | Asp, Gln |
| Glycine, Gly, G | Ala |
| Histidein, His, H | Asn, Gln |
| Isoleucine, Ile, I | Leu, Val |
| Leucine, Leu, L | Ile, Val |
| Lysine, lys, K | Ar, His |
| Methionine, Met, M | Leu, Ile, Tyr |
| Phenylalanine, Phe, F | Tyr, Met, Leu |
| Proline, Pro, P | Ala |
| Serine, Ser, S | Thr |
| Threonine, Thr, T | Ser |
| Tryptophan, Trp, W | Tyr, Phe |
| Tyrosine, Tyr, Y | Try, Phe |
| Valine, Val, V | Ile, Leu |

Antibodies described herein may comprise suitable Fc regions. In one embodiment, the antibody or antigen-binding portion thereof comprises an Fc region, for example a canine Fc region, for example a canine IgGB Fc region. In one embodiment, the Fc portion of the antibody may be modified. For example, modifications may be made to the Fc region to improve certain properties, e.g. to provide reduced complement- and FcγR-mediated effector functions.

Exemplary modified Fc regions for canine antibodies of the invention based on a canine IgG-B Fc region are provided in SEQ ID Nos 722 to 730 which show the IgG-B constant region, including the Fc region. These sequences comprise modifications compared to wild type Fc IgG-B regions. The modified Fc regions reduce or abolish canine IgG-B effector function when compared to the same polypeptide comprising a wild-type IgG-B Fc domain. This is shown in the examples. The amino acid substitutions reside in the lower hinge, proline sandwich region and SHED region. Thus, an antibody of the invention may include a modified Fc region having the modifications as shown in SEQ ID Nos 722 to 730. Modifications are with reference to the wt sequence as shown in SEQ ID NO. 721.

Thus, with reference to the wild type residue in canine IgG-B constant region (SEQ ID NO: 721), the antibodies may have the following amino acid substitutions at the following positions in the Fc domain:
E119G;
M120S or A;
L121A;
D153G;
P154R:
D156N;
N211H;
K212I;
A213G;
P215G and/or
P217S.

In one embodiment, the Fc region comprises
a) an amino acid substitution at position 120 of SEQ ID NO: 721 to S and
b) an amino acid substitution at position 211 of SEQ ID NO: 721 to H, an amino acid substitution at position 212 of SEQ ID NO: 721 to I and an amino acid substitution at position 213 of SEQ ID NO: 721 to G.

In one embodiment, the Fc region comprises
a) an amino acid substitution at position 120 of SEQ ID NO: 721 to S;
b) an amino acid substitution at position 153 of SEQ ID NO: 721 to G and an amino acid substitution at position 154 of SEQ ID NO: 721 to R and
c) an amino acid substitution at position 211 of SEQ ID NO: 721 to H, an amino acid substitution at position 212 of SEQ ID NO: 721 to I and an amino acid substitution at position 213 of SEQ ID NO: 721 to G.

In one embodiment, the Fc region comprises
a) an amino acid substitution at position 120 of SEQ ID NO: 721 to S;
b) an amino acid substitution at position 153 of SEQ ID NO: 721 to G and an amino acid substitution at position 154 of SEQ ID NO: 721 to R and
c) an amino acid substitution at position 211 of SEQ ID NO: 721 to H, an amino acid substitution at position 212 of SEQ ID NO: 721 to I and an amino acid substitution at position 213 of SEQ ID NO: 721 to G and an amino acid substitution at position 217 of SEQ ID NO: 721 to S.

In one embodiment, the Fc region comprises
a) an amino acid substitution at position 120 of SEQ ID NO: 721 to S;
b) an amino acid substitution at position 153 of SEQ ID NO: 721 to G and an amino acid substitution at position 154 of SEQ ID NO: 721 to R and
c) an amino acid substitution at position 211 of SEQ ID NO: 721 to H, an amino acid substitution at position 212 of SEQ ID NO: 721 to I and an amino acid substitution at position 213 of SEQ ID NO: 141 to G and an amino acid substitution at position 215 of SEQ ID NO: 721 to G.

In one embodiment, the Fc region comprises an amino acid substitution at position 120 of SEQ ID NO: 721 to A and at position 121 to A.

In one embodiment, the Fc region comprises
a) an amino acid substitution at position 120 of SEQ ID NO: 721 to A and at position 121 to A and
b) an amino acid substitution at position 217 of SEQ ID NO: 721 to S.

In one embodiment, the Fc region comprises
a) an amino acid substitution at position 120 of SEQ ID NO: 721 to A and at position 121 to A and
b) an amino acid substitution at position 215 of SEQ ID NO: 721 to G.

In one embodiment, the Fc region comprises
a) an amino acid substitution at position 119 of SEQ ID NO: 721 to G and
b) an amino acid substitution at position 156 of SEQ ID NO: 721 to N.

In one embodiment, the Fc region comprises
a) an amino acid substitution at position 120 of SEQ ID NO: 721 to A and at position 121 to A;
b) an amino acid substitution at position 156 of SEQ ID NO: 721 to N and
c) an amino acid substitution at position 217 of SEQ ID NO: 721 to S.

In another aspect, there are provided binding molecules, e.g. antibodies, antibody fragments or antibody mimetics that bind at or near the same epitope or an overlapping epitope on the companion animal OX40L or OX40 respectively (e.g. dog OX40 or OX40L) as any of the OX40L/OX40 antibodies of the invention (i.e., antibodies that have the ability to cross-compete for binding to OX40L/OX40 with an antibody of the invention). The antibodies of the invention can thus be used as a reference antibody.

Such cross-competing antibodies can be identified based on their ability to cross-compete with an antibody described herein in standard OX40L/OX40 binding assays. For example, SPR analysis such as BIAcore® analysis, BLI analysis such as FortBio Octet®, ELISA assays or flow cytometry may be used to demonstrate cross-competition with the antibodies.

In one embodiment, there is provided a binding agent capable of binding companion animal OX40L or OX40 respectively (e.g. dog OX40 or OX40L) wherein an antibody of the invention displaces the binding agent in a competitive assay.

Included within the scope of this invention are antibody derivatives. A “derivative” of an antibody contains additional chemical moieties not normally a part of the protein. Covalent modifications of the protein are included within the scope of this invention. Such modifications may be introduced into the molecule by reacting targeted amino acid residues of the antibody with an organic derivatizing agent that is capable of reacting with selected side chains or terminal residues. For example, derivatization with bifunctional agents, well-known in the art, is useful for cross-linking the antibody or fragment to a water-insoluble support matrix or to other macromolecular carriers.

The antibody or fragment thereof as described herein can be used as a building block in a multispecific, for example bispecific or trispecific, binding agent that provides dual targeting of a companion animal OX40L or OX40 respectively (e.g. dog OX40 or OX40L) expressing cell. Thus, the antibody or fragment thereof as described herein is linked to another therapeutic entity that targets a different antigen. This other therapeutic entity is for example selected from an antibody or antibody fragment (e.g., a Fab, F(ab′)2, Fv, a single chain Fv fragment (scFv) or single domain antibody, for example a VH or VHH domain), CDR region or antibody mimetic protein. Suitable non-immunogenic linker peptides are known in the art, for example, linkers that include G and/or S residues, (G4S)n, (SG4)n or G4(SG4)n peptide linkers, wherein “n” is generally a number between 1 and 10.

In another embodiment, the antibody or fragment thereof according to the invention is linked to a further moiety that may serve to prolong the half-life of the molecule. The further moiety may comprise a protein, for example an antibody, or part thereof that binds a serum albumin, e.g., dog or cat serum albumin. Other modifications that prolong half-life are also known and include, for example, modification by PEG or by incorporation in a liposome.

In one embodiment, the antibody or fragment thereof according to the invention is labelled with a detectable or functional label. A label can be any molecule that produces or can be induced to produce a signal, including but not limited to fluorophores, fluorescers, radiolabels, enzymes, chemiluminescers, a nuclear magnetic resonance active label or photosensitizers. Thus, the binding may be detected and/or measured by detecting fluorescence or luminescence, radioactivity, enzyme activity or light absorbance.

In still other embodiments, the antibody or fragment thereof according to the invention is coupled to a toxin. In one embodiment, the therapeutic moiety is a toxin, for example a cytotoxic radionuclide, chemical toxin or protein toxin.

The term “half-life” as used herein refers to the time taken for the serum concentration of the amino acid sequence, compound or polypeptide to be reduced by 50%, in vivo, for example due to degradation of the sequence or compound and/or clearance or sequestration of the sequence or compound by natural mechanisms. Half-life may be increased by at least 1.5 times, preferably at least 2 times, such as at least 5 times, for example at least 10 times or more than 20 times, greater than the half-life of the corresponding antibodies of the invention. For example, increased half-life may be more than 1 hours, preferably more than 2 hours, more preferably more than 6 hours, such as more than 12 hours, or even more than 24, 48 or 72 hours, compared to the corresponding antibodies of the invention. The in vivo half-life of an amino acid sequence, compound or polypeptide of the invention can be determined in any manner known per se, such as by pharmacokinetic analysis. Suitable techniques will be clear to the person skilled in the art. Half-life can for example be expressed using parameters such as the t½-alpha t½-beta and the area under the curve (AUC).

The antibodies and fragments of the invention may also be used in cell therapy, for example chimeric antigen receptor T-cell (CAR-T) therapy.

### Nucleic Acids

In another aspect, the invention relates to a nucleic acid sequence encoding an amino acid sequence of an antibody or antibody fragment as described herein.

In one embodiment, the nucleic acid encodes a HC variable region which comprises or consists of a sequence selected from SEQ ID No. 11, SEQ ID No. 21, SEQ ID No. 31, SEQ ID No. 41, SEQ ID No. 51, SEQ ID No. 61, SEQ ID No. 71, SEQ ID No. 81, SEQ ID No. 91, SEQ ID No. 101, SEQ ID No. 111, SEQ ID No. 121, SEQ ID No. 131, SEQ ID No.141, SEQ ID No. 151, SEQ ID No.161, SEQ ID No.171, SEQ ID No.181, SEQ ID No.191, SEQ ID No. 201, SEQ ID No. 211, SEQ ID No. 221, SEQ ID No. 231, SEQ ID No. 241, SEQ ID No. 251, SEQ ID No. 261, SEQ ID No. 271, SEQ ID No. 281, SEQ ID No. 291, SEQ ID No.301, SEQ ID No. 311, SEQ ID No. 321, SEQ ID No. 331, SEQ ID No. 341, SEQ ID No. 351, SEQ ID No. 361, SEQ ID No. 371, SEQ ID No. 381, SEQ ID No.391, SEQ ID No.401, SEQ ID No. 411, SEQ ID No.421, SEQ ID No. 431, SEQ ID No. 441, SEQ ID No. 451, SEQ ID No. 461, SEQ ID No. 471, SEQ ID No. 481, SEQ ID No. 491, SEQ ID No. 501, SEQ ID No. 511, SEQ ID No. 521, SEQ ID No. 531, SEQ ID No. 541, SEQ ID No. 551, SEQ ID No. 561, SEQ ID No. 571, SEQ ID No. 581, SEQ ID No. 591, SEQ ID No. 601, SEQ ID No. 611, SEQ ID No. 621, SEQ ID No. 631, SEQ ID No. 641, SEQ ID No. 651, SEQ ID No. 661, SEQ ID No. 671, SEQ ID No. 681, SEQ ID No. 691, SEQ ID No.701, SEQ ID No. 711, SEQ ID No. 737, SEQ ID No. 747, SEQ ID No. 757, SEQ ID No. 767. SEQ ID No. 777. SEQ ID No. 787.

### SEQ ID No. 797.

In one embodiment, the nucleic acid encodes a LC variable region which comprises or consists of a sequence selected from SEQ ID No. 13, SEQ ID No. 23, SEQ ID No. 33, SEQ ID No. 43, SEQ ID No. 53, SEQ ID No. 63, SEQ ID No. 73, SEQ ID No. 83, SEQ ID No. 93, SEQ ID No. 103, SEQ ID No. 113, SEQ ID No. 123, SEQ ID No. 133, SEQ ID No. 143, SEQ ID No.153, SEQ ID No. 163, SEQ ID No.173, SEQ ID No.183, SEQ ID No.193, SEQ ID No. 203, SEQ ID No. 213, SEQ ID No. 223, SEQ ID No. 233, SEQ ID No. 243, SEQ ID No. 253, SEQ ID No. 263, SEQ ID No. 273, SEQ ID No. 283, SEQ ID No. 293, SEQ ID No.303, SEQ ID No. 313, SEQ ID No. 323, SEQ ID No. 343, SEQ ID No. 353, SEQ ID No. 363, SEQ ID No. 373, SEQ ID No. 383, SEQ ID No. 393, SEQ ID No. 403, SEQ ID No. 413, SEQ ID No. 423, SEQ ID No. 433, SEQ ID No. 443, SEQ ID No. 453, SEQ ID No. 463, SEQ ID No. 473, SEQ ID No. 483, SEQ ID No. 493, SEQ ID No.503, SEQ ID No. 513, SEQ ID No. 523, SEQ ID No. 533, SEQ ID No. 543, SEQ ID No. 553, SEQ ID No. 563, SEQ ID No. 573, SEQ ID No. 583, SEQ ID No. 593, SEQ ID No. 603, SEQ ID No. 613, SEQ ID No. 623, SEQ ID No. 633, SEQ ID No. 643, SEQ ID No. 653, SEQ ID No. 663, SEQ ID No.673, SEQ ID No. 683, SEQ ID No. 693, SEQ ID No. 703, SEQ ID No. 713, SEQ ID No. 739, SEQ ID No. 749, SEQ ID No. 759, SEQ ID No. 769, SEQ ID No. 779, SEQ ID No. 789 or SEQ ID No. 799.

### Exemplary Methods for Making the Antibody

An antibody or fragment described herein can be obtained from a mammal, for example a rodent, for example a transgenic animal, that expresses antibodies upon stimulation with an OX40L or OX40 antigen of the target companion animal, e.g. dog or cat OX40L or OX40. Suitably companion animal antibody genes have been introduced such that companion animal antibodies are generated. The transgenic rodent, for example a mouse, preferably has a reduced capacity to express endogenous antibody genes. Thus, in one embodiment, the rodent has a reduced capacity to express endogenous light and/or heavy chain antibody genes. The rodent, for example a mouse, may therefore comprise modifications to disrupt expression of endogenous kappa and lambda light and/or heavy chain antibody genes so that no functional mouse light and/or heavy chains are produced, for example as further explained below. Such transgenic rodents are described in the art and this is further explained in the examples below.

Other methods may involve speciation of a mouse monoclonal typically following the steps of immunising a mouse with an OX40L or OX40 antigen of the target companion animal, isolating B cells and fusing them to a fusion partner cell line, isolating mouse monoclonal antibodies by selection. Speciation is then carried out by chimerization and/or further informatic-guided speciation. Strategies for speciation, such as strategies for caninization or felinization, are described, for example, in WO2013/011407, see Example 5.

In other methods, a set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art (see for example Phage Display of Peptides and Proteins: A Laboratory Manual, Academic Press; 1st edition (Oct. 28, 1996) Brian K. Kay, Jill Winter, John McCafferty).

It is also possible to generate libraries, for example phage libraries, by isolating a cell or tissue expressing an antigen-specific, heavy chain-only antibody, cloning the sequence encoding the V_{H} domain(s) from mRNA derived from the isolated cell or tissue and displaying the encoded protein using a library. The antibody or fragment can be expressed in bacterial, yeast or other expression systems.

### Exemplary Therapeutic Applications

In one aspect, we provide an antibody or fragment as described herein for use in the treatment of disease.

In another aspect, there is provided a pharmaceutical composition comprising an antibody or fragment as described herein and optionally a pharmaceutically acceptable carrier. The term pharmaceutical composition as used herein refers to a composition that is used to treat a companion animal, that is for veterinary use, i.e. a veterinary composition. In preferred embodiments the animal that is treated is a dog or a cat.

An antibody or fragment thereof as described herein or the pharmaceutical composition of the invention can be administered by any convenient route, including but not limited to oral, topical, parenteral, sublingual, rectal, vaginal, ocular, intranasal, pulmonary, intradermal, intravitreal, intramuscular, intraperitoneal, intravenous, subcutaneous, intracerebral, transdermal, transmucosal, by inhalation, or topical, particularly to the ears, nose, eyes, or skin or by inhalation.

In one embodiment, administration is subcutaneous. The concentration of the antibody or antibody fragment may be 10 to 50 mg/ml, e.g. 10, 20, 30, 40 or 50 mg/ml. In one embodiment, the concentration is 25 to 35 mg/ml, for example about 30 mg/ml. In one embodiment, the antibody is provided as a dose in 1 ml of solution.

Parenteral administration includes, for example, intravenous, intramuscular, intraarterial, intraperitoneal, intranasal, rectal, intravesical, intradermal, topical or subcutaneous administration. Preferably, the compositions are administered parenterally.

The pharmaceutically acceptable carrier or vehicle can be particulate, so that the compositions are, for example, in tablet or powder form. The term “carrier” refers to a diluent, adjuvant or excipient, with which a drug antibody conjugate of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the antibody or fragment thereof of the present invention or compositions and pharmaceutically acceptable carriers are sterile. Water is a preferred carrier when the drug antibody conjugates of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition of the invention can be in the form of a liquid, e.g., a solution, emulsion or suspension. The liquid can be useful for delivery by injection, infusion (e.g., IV infusion) or subcutaneously.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

The composition can be in the form of a liquid, e. g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units.

In specific embodiments, it can be desirable to administer the composition locally to the area in need of treatment, or by injection, intravenous injection or infusion.

The amount of the therapeutic that is effective/active in the treatment of a particular disorder or condition will depend on the nature of the disorder or condition and the animal to be treated and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each subject's circumstances. Factors like age, body weight, sex, diet, time of administration, rate of excretion, condition of the host, drug combinations, reaction sensitivities and severity of the disease shall be taken into account.

Typically, the amount is at least about 0.01% of an antibody of the present invention by weight of the composition. When intended for oral administration, this amount can be varied to range from about 0.1% to about 80% by weight of the composition. Preferred oral compositions can comprise from about 4% to about 50% of the antibody of the present invention by weight of the composition.

Preferred compositions of the present invention are prepared so that a parenteral dosage unit contains from about 0.01% to about 2% by weight of the antibody of the present invention.

For administration by injection, the composition can comprise from about typically about 0.1 mg/kg to about 250 mg/kg of the subject's body weight, preferably, between about 0.1 mg/kg and about 20 mg/kg of the animal's body weight, and more preferably about 1 mg/kg to about 10 mg/kg of the animal's body weight. In one embodiment, the composition is administered at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, 4, 5, 6, 7, 8 or more weeks.

In one embodiment, post-treatment, the subject has at least 7 days, or at least 14 days, or at least 21 days, or at least 28 days, or at least 40 days, or at least 50 days, or at least 60 days disease progression-free.

In one embodiment, the number of days of survival, the number of disease-free days, or the number of disease-progression free days is at least 2 months, or at least 3 months, or at least 4 months, e.g. at least 5 months, such as at least 6 months.

In one embodiment, the number of days of survival, the number of disease-free days, or the number of disease-progression free days is at least 9 months, or at least one year. The invention provides methods of treating or preventing OX40L/OX40-mediated diseases or disorders in a companion animal, e.g., a dog, cat or horse, comprising administering an effective amount of an antibody or fragment of the present invention to the animal in need thereof.

As used herein, “treat”, “treating” or “treatment” means inhibiting or relieving a disease or disorder. For example, treatment can include a postponement of development of the symptoms associated with a disease or disorder, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., companion animal patients, being treated. Many medical treatments are effective for some, but not all, patients that undergo the treatment.

Suitably, for treating a condition such as atopic dermatitis, post-treatment, a reduction in pruritus score is observed. Suitable methods for measuring pruritus score will be known to those skilled in the art.

In one embodiment, an antagonistic antibody in accordance with the invention, or fragment thereof, suppresses a GVHD reaction in a xenogenic graft versus host reaction such as described in WO2013/0008171. In an embodiment the antibody in accordance with the invention or a fragment thereof shows activity in an antigen recall study similar to what set out in Saghari M. et al, Clin Pharmacol Ther. 2022 Jan. 29. doi: 10.1002/cpt.2539 or a house dust mites canine model as set out in Marsella R. et al, Vet. Dermatol 2006 February; 17(1):24-35. doi: 10.1111/j.1365-3164.2005.00496.x.

The term “subject” or “patient” refers to a companion animal which is the object of treatment, observation, or experiment. By way of example only, a subject includes, but is not limited to, a dog, cat or horse. For the avoidance of doubt, the treatment of humans is excluded.

In one embodiment, a “patient” is one which is non-responsive to Cytopoint or Apoquel.

As used herein, the term “effective amount” means an amount of an anti-OX40L/OX40 antibody, that when administered alone or in combination with an additional therapeutic agent to a cell, tissue, or subject, is effective to achieve the desired therapeutic or prophylactic effect under the conditions of administration

In another aspect, the invention relates to the use of an antibody or fragment as described herein, or pharmaceutical composition of the invention in the manufacture of a medicament for the treatment or prevention of a disease as defined herein.

As mentioned above, the antibody or fragment according to the invention is useful in the treatment or prevention of an OX40L/OX40 mediated disease.

The term OX40L/OX40 mediated disease refers to any disease or disorder that is mediated by the OX40L/OX40 signalling pathway and which can be treated/alleviated by targeting the OX40L/OX40 antigen. An OX40L-mediated disease and OX40-mediated disease refer to any disease or condition that is completely or partially caused by or is the result of OX40L or OX40 respectively. In certain embodiments, OX40L or OX40 is aberrantly (e.g., highly) expressed on the surface of a cell. In some embodiments, OX40L or OX40 may be aberrantly upregulated on a particular cell type. In other embodiments, normal, aberrant or excessive cell signalling is caused by binding of OX40 to OX40L.

Evidence in humans suggests that disruption of the OX40/OX40L axis reduces T cell activation, proliferation and memory T cell establishment, and as such an anti-OX40L/OX40 antibody could be used to prevent, treat or ameliorate the symptoms of a number of diseases, including atopic dermatitis, type 1 diabetes, Chron's disease, ulcerative colitis and other autoimmune diseases.

In one embodiment the anti-OX40L/OX40 antibody is used to prevent treat or ameliorate symptoms of a disease. In one embodiment, the disease such is selected from allergy, asthma, and diseases associated with autoimmunity and inflammation, which includes multiple sclerosis, rheumatoid arthritis, inflammatory bowel disease, graft-versus-host disease, experimental autoimmune encephalomyelitis (EAE), experimental leishmaniasis, collagen-induced arthritis, colitis (such as ulcerative colitis), contact hypersensitivity reactions, type 1 diabetes, Crohn's Disease, and Grave's Disease. In one embodiment the disease is an inflammatory condition which refers to pathological states resulting in inflammation or an autoimmune disease. In particular, the disease is selected from the following non-limiting list: inflammatory skin diseases, including atopic dermatitis (atopy), allergic dermatitis, pruritus, psoriasis, scleroderma, or eczema; responses associated with inflammatory bowel disease (such as Crohn's disease and ulcerative colitis); type 1 diabetes; ischemic reperfusion; adult respiratory distress syndrome; asthma; meningitis; encephalitis; uveitis; autoimmune diseases such as rheumatoid arthritis, Sjorgen's syndrome, vasculitis; diseases involving leukocyte diapedesis; central nervous system (CNS) inflammatory disorder, multiple organ injury syndrome secondary to septicaemia or trauma; bacterial pneumonia, antigen-antibody complex mediated diseases; inflammations of the lung, including pleurisy, alveolitis, vasculitis, pneumonia, chronic bronchitis, bronchiectasis, and cystic fibrosis; etc. Other diseases include equine indications such as sweet itch, summer recurrent dermatitis or equine insect bite hypersensitivity.

Exemplary Combinations with Other Agents

The antibodies, antibody fragments or pharmaceutical composition of the invention may be administered as the sole active ingredient or in combination with one or more other therapeutic agent. A therapeutic agent is a compound or molecule which is useful in the treatment of a disease.

Examples of therapeutic agents include antibodies, antibody fragments, drugs, toxins, nucleases, hormones, an anti-inflammatory agent, immunomodulators, pro-apoptotic agents, anti-angiogenic agents, boron compounds, photoactive agents or dyes and radioisotopes. An antibody molecule includes a full antibody or fragment thereof (e.g., a Fab, F(ab′)2, Fv, a single chain Fv fragment (scFv) or a single domain antibody, for example a VH domain, or antibody mimetic protein.

In one embodiment, the antibody or antibody fragment or the pharmaceutical composition described herein is used in combination with an existing therapy or therapeutic agent. Thus, in another aspect, the invention also relates to a combination therapy comprising administration of the antibody or antibody fragment or the pharmaceutical composition described herein and another therapy.

In one embodiment, the therapeutic agent is selected from the following non-limiting list: rapamycin (sirolimus), tacrolimus, cyclosporin, corticosteroids (e.g. methylprednisolone), methotrexate, mycophenolate mofetil, anti-CD28 antibodies, anti-IL12/IL-23 antibodies, anti-CD20 antibodies, anti-CD30 antibodies, CTLA4-Fc molecules, CCR5 receptor antagonists, anti-CD40L antibodies, anti-VI_A4 antibodies, anti-LFA1 antibodies, fludarabine, anti-CD52 antibodies, anti-CD45 antibodies, cyclophosphamide, anti-thymocyte globulins, anti-complement C5 antibodies, anti-a4b7 integrin antibodies, anti-IL6 antibodies, anti-IL6-R antibodies, anti-IL2R antibodies, anti-CD25 antibodies, anti-TNFa/TNFa-Fc molecules, HDAC inhibitors, JAK inhibitors, such as JAK-1 and JAK-3 inhibitors, anti-IL-31 antibodies, SYK inhibitors, anti-IL-4Ra antibodies, anti-IL-13 antibodies, anti-TSLP antibodies, and PDE4 inhibitors lokietmab (Cytopoint®), cyclosporin (Atopica®) and oclacitinib (Apoquel®). In some embodiments, the antibody or antibody fragment or the pharmaceutical composition described herein may be administered with two or more therapeutic agents.

The antibody or antibody fragment or the pharmaceutical composition described herein may be administered at the same time or at a different time as the other therapy or therapeutic compound or therapy, e.g., simultaneously, separately or sequentially.

### Exemplary Kits

In another aspect, the invention provides a kit for the treatment or prevention of a disease for example as listed herein and/or for detecting OX40L/OX40 for diagnosis, prognosis or monitoring disease comprising an antibody or fragment of the invention. Such a kit may contain other components, packaging, instructions, or material to aid in the detection of OX40L/OX40 protein. The kit may include a labelled antibody that binds to OX40L/OX40 or a binding molecule comprising an antibody that binds to OX40L/OX40 and one or more compounds for detecting the label.

The invention in another aspect provides an antibody or fragment thereof that binds to OX40L/OX40, or pharmaceutical composition described herein packaged in lyophilized form, or packaged in an aqueous medium.

### Exemplary Non Therapeutic Applications

In another aspect, an antibody or fragment that binds to OX40L/OX40 as described herein is used for non-therapeutic purposes, such as diagnostic tests and assays. A method for detecting the presence of a companion animal OX40L/OX40 in a test sample comprises contacting said sample with an antibody or fragment thereof as described herein and at least one detectable label and detecting binding of said antibody to a companion animal OX40L/OX40.

Modifications of antibodies for diagnostic purposes are well known in the art. For example, antibodies may be modified with a ligand group such as biotin, or a detectable marker group such as a fluorescent group, a radioisotope, or an enzyme. Compounds of the invention can be used for diagnostic purposes and e.g. labelled using conventional techniques. Suitable detectable labels include but are not limited to fluorophores, chromophores, radioactive atoms, electron-dense reagents, enzymes, and ligands having specific binding partners.

In some embodiments, the binding of antigen to antibody is detected without the use of a solid support. For example, the binding of antigen to antibody can be detected in a liquid format.

In other embodiments, an antibody or fragment can, for example, be fixed to nitrocellulose, or another solid support which is capable of immobilizing cells, cell particles or soluble proteins. The support can then be washed with suitable buffers followed by treatment with the detectably labelled antibody. The solid phase support can then be washed with the buffer a second time to remove unbound peptide or antibody. The amount of bound label on the solid support can then be detected by known method steps. “Solid phase support” or “carrier” refers to any support capable of binding peptide, antigen, or antibody. Well-known supports or carriers, include glass, polystyrene, polypropylene, polyethylene, polyvinylidenefluoride (PVDF), dextran, nylon, amylases, natural and modified celluloses, polyacrylamides, agaroses, and magnetite. The nature of the carrier can be either soluble to some extent or insoluble for the purposes of the present invention. The support material can have virtually any possible structural configuration so long as the coupled molecule is capable of binding to OX40L/OX40 or an anti-OX40L/OX40 antibody. Thus, the support configuration can be spherical, as in a bead, or cylindrical, as in the inside surface of a test tube, or the external surface of a rod. Alternatively, the surface can be flat, such as a sheet, culture dish, test strip, etc. For example, supports may include polystyrene beads. Those skilled in the art will know many other suitable carriers for binding antibody, peptide or antigen, or can ascertain the same by routine experimentation. Well known method steps can determine binding activity of a given lot of anti-OX40L/OX40 antibody. Those skilled in the art can determine operative and optimal assay conditions by routine experimentation.

For the purposes of the present invention, the OX40L/OX40 which is detected by the above assays can be present in a biological test sample. Any sample containing OX40L/OX40 may be used. For example, the sample is a biological fluid such as, for example, blood, serum, lymph, urine, feces, inflammatory exudate, cerebrospinal fluid, amniotic fluid, a tissue extract or homogenate, and the like.

### Nucleic Acid Construct and Hosts Cells

Furthermore, the invention relates to a nucleic acid construct comprising at least one nucleic acid as defined herein, i.e. nucleic acid molecules encoding antibodies of the invention or nucleic acid molecules encoding an OX40 protein. The construct may be in the form of a plasmid, vector, transcription or expression cassette.

An expression vector can be, for example, a plasmid, such as pBR322, pUC, or Co1E1, or an adenovirus vector, such as an adenovirus Type 2 vector or Type 5 vector. Vectors suitable for use in the present invention include, for example, bacterial vectors, mammalian vectors, viral vectors (such as retroviral, adenoviral, adeno-associated viral, herpes virus, simian virus 40 (SV40), and bovine papilloma virus vectors) and baculovirus-derived vectors for use in insect cells. Polynucleotides in such vectors are preferably operably linked to a promoter, which is selected based on, e.g., the cell type in which expression is sought.

The expression vector can be transferred to a host cell by conventional techniques and the transfected cells are then cultured by conventional techniques to produce an antibody of the invention. The invention includes host cells containing polynucleotides encoding an antibody of the invention (e.g., whole antibody, a heavy or light chain thereof, or portion thereof, or a single chain antibody of the invention, or a fragment or variant thereof), operably linked to a heterologous promoter. For the expression of entire antibody molecules, vectors encoding both the heavy and light chains may be co-expressed in the host cell for expression of the entire immunoglobulin molecule. In one embodiment, the expression vector is one which enables heterologous expression e.g. expression in a host organism from different species.

The invention also relates to an isolated recombinant host cell comprising one or more nucleic acid construct as described herein, i.e. nucleic acid molecules encoding antibodies of the invention or nucleic acid molecules encoding an OX40 protein. Host cells useful in the present invention are prokaryotic, yeast, or higher eukaryotic cells and include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g. Baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

Prokaryotes useful as host cells in the present invention include gram negative or gram-positive organisms such as E. coli, B. subtilis, Enterobacter, Erwinia, Klebsiella, Proteus, Salmonella, Serratia, and Shigella, as well as Bacilli, Pseudomonas, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting. In one embodiment, a method of making an anti-OX40 antibody as described herein is provided, wherein the method comprises culturing the host cell under conditions suitable for expression of the polynucleotide encoding the antibody and isolating the antibody.

In some embodiments, the nucleic acid may also comprise a leader sequence. Any suitable leader sequences may be used, including the native immunoglobulin germline leader sequence, or others, such as the Campath leader sequence (see U.S. Pat. No. 8,362,208B2), may be chosen to enhance protein expression.

### Assays and Expression Systems

The invention also relates to a heterologous assay or expression system comprising a companion animal (e.g. dog) OX40 and a cell line derived from a different species, e.g. a human cell line such as HEK.

The assay comprises contacting a companion animal (e.g. dog) OX40 with a cell line derived from a different species, e.g. a cell line from a different mammal, e.g. a rodent cell line or a human cell line such as HEK. For example, the cell line is transfected with companion animal (e.g. dog) OX40 such that it expresses companion animal (e.g. dog) OX40 in a stable or transient manner.

The cell line may comprise a reporter gene and activation of OX40 by OX40L can be assessed using the reporter gene, e.g. by quantification of reporter gene expression. Suitable reporter genes are known to the skilled person and may include fluorescent markers or alkaline phosphatase (SEAP).

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, ABAB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The present invention is described in more detail in the following non-limiting examples.

### EXAMPLES

### Example 1: Cloning Canine OX40L and OX40

### OX40L Sequence Prediction

The nucleotide sequence and gene structure for canine OX40L was predicted based on human OX40L nucleotide sequence alignment to canine CamFam3.1 genome assembly from the UCSC Genome Browser (University of Santa Cruz). This gave 3 predicted exons (FIG. 1A). Unlike many transmembrane proteins which consist of a 5′ terminus followed by the extracellular domain, OX40L protein structure is reversed (see FIG. 1B).

### OX40 Sequence Prediction

The nucleotide sequence for canine OX40 was predicted based on nucleotide sequence homology alignment of human OX40 to the canine reference genome. This gave 7 predicted exons with extracellular domain at the N terminus (FIG. 1A).

### OX40L and OX40 Cloning

The sequence for both OX40L and OX40 were confirmed by cloning from PBMCs isolated from beagle blood using standard protocols. Beagle whole blood was supplied by Envigo RMS (Alconbury, Huntingdon, UK) and PBMCs were isolated using a Ficoll gradient. Briefly, 10 ml whole blood was diluted with 25 ml phosphate buffered saline (PBS) and layered onto 15 ml Ficoll Paque Plus (Sigma Aldrich) before centrifuging at 800 rcf for 10 min at room temperature, with slow acceleration and no brake. The interphase disk, which represents the PBMC fraction was collected into PBS. The PBMCs were counted and resuspended at 10⁶ cells ml-1 in RPMI media (Sigma Aldrich) supplemented with 1 μg ml⁻¹ canine IL-2.

To stimulate OX40 and OX40L expression, 5 mg ml⁻¹ Phytohaemagglutinin-L (PHA-L; Fisher Scientific) was added, and PBMC's were harvested 1 and 4 days later. Total RNA was isolated from activated PBMC's with the QIAGEN RNeasy Mini Kit (Qiagen, Hilden, DE), yielding 160 μg-440 μg RNA following an on-column DNAse digestion. Specific cDNA amplification of OX40 and OX40L was undertaken using the SuperScript IV One-Step RT-PCR kit (ThermoFisher, Massachusetts, US) with 3′ and 5′ terminal primers generated from predicted OX40 and OX40L nucleotides sequences. Primer sequences are shown in Table 2 (SEQ ID NO: 7 to 10).

OX40 and OX40L RT-PCR products were subcloned into pJET using the CloneJET PCR Cloning Kit (ThermoFisher), before being transferred to pcDNA3.1 for mammalian expression.

The nucleotide and amino acid sequence for canine OX40L (sequence of cloned OX40L/Full length membrane form for cellular expression is shown in Table 2 (SEQ ID NO:1 and 2).

For OX40, the presence of potential splice variants in the RT-PCR products was observed. To isolate the individual splice variant transcripts, the RT-PCR products were subcloned and two different transcripts were identified with sequences as shown in Table 2 (nucleotide, amino acid translation), the short and the long variant (SEQ ID NO:3 to 6).

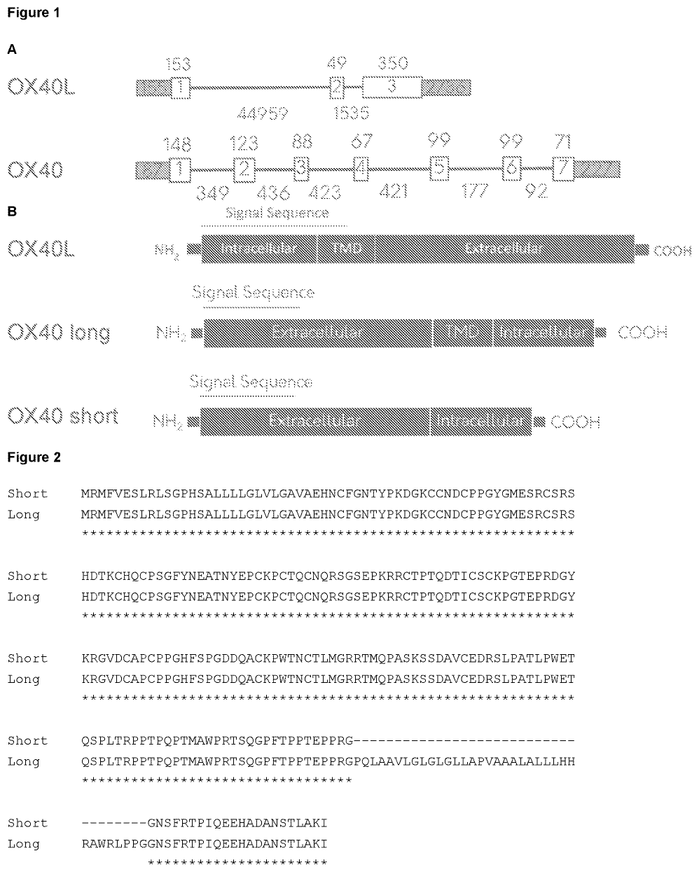

FIG. 2 shows an alignment of these splice variants. The long splice variant of OX40 consists of exons arranged in the sequence 1-2-3-4-5-6-7. The short splice variant does not contain exon 6, and has the arrangement 1-2-3-4-5-7

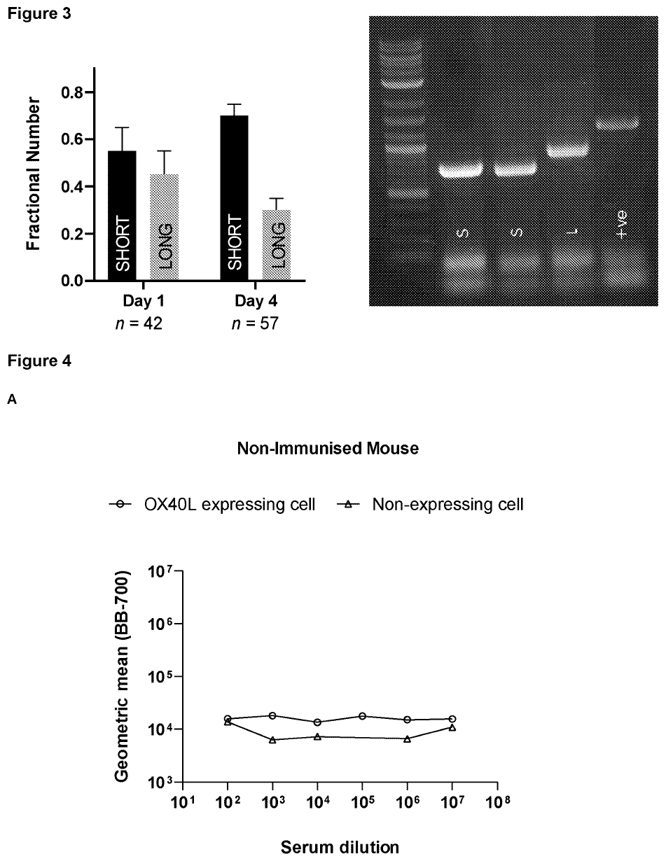

The relative abundance of OX40 splice-variants were assessed using a single-step PCR of pJET-OX40 colonies. Briefly, single colonies were picked from LB-amp plates into 10 μl OneTaq Quick-Load PCR mix (NEB) and amplified with the following PCR cycle: 1 cycle of 94° C. for 30 s; 30 cycles of 94° C. for 30 s, 61° C. for 30 s, 68° C. for 2 min; 1 cycle of 68° C. for 5 min. The abundance of the two splice variants was determined by assessing the relative lengths of the resultant PCR products (FIG. 3). The short splice variant codes for a truncated product. As this product lacks the transmembrane region in its entirety, it seems most likely that the resultant short variant would be secreted.

### Example 2: Generation of Canine OX40 and OX40L Stably Expressing Cells

Human embryonic kidney (HEK) 293 cells were grown on 90 mm round tissue culture plates as monolayers in DMEM/F12 (Life Technologies, California, US) supplemented with 10% fetal bovine serum (FBS; Sigma Aldrich) at 37° C. in a moist atmosphere containing 5% CO₂. HEK293 cells were transfected with plasmids encoding either OX40 (SEQ. ID 5) or OX40L (SEQ. ID 2) cDNA using polyethyleneimine (PEI MAX: 40 kDa, Polysciences Inc., Eppelheim, Germany). 30 μl of PEI MAX (1 mg ml⁻¹), 5 μg cDNA and 1 ml DMEM/F12 were incubated for 10 min at room temperature, added dropwise to a 90 mm plate of 70-80% confluent HEK293 cells, and incubated for 2-3 days. Stably transfected cells were selected 48 hrs later using a suitable antibiotic.

Mouse embryonic fibroblasts (MEF) were grown on 90 mm round tissue culture plates as monolayers in DMEM-high glucose (Life Technologies) supplemented with 10% FBS, 10% β-mercaptoethanol and 10% non-essential amino acids at 37° C. in a moist atmosphere and 5% CO₂. Cells were transfected with plasmids encoding OX40L cDNA (SEQ. ID 2) using Lipofectamine LTX (ThermoFisher Scientific, Waltham, MA, USA) according to the manufacturer's recommended instructions. Stably transfected cells were selected 48 hrs later using a suitable antibiotic.

### Example 3: Canine OX40L Immunisation Using Ky9™ Transgenic Platform

Ky9™ mice, substantially as described in WO2018/189520 and WO2020/074874, for example, were immunised with OX40L expression DNA construct or MEFs with OX40L stably expressed. The transgenic mice have been modified compared to wild type mice by insertion of immunoglobulin heavy (IGH) chain and light chain (IGL) variable (V) region genes, IGH D region genes and IGH and IGL J region genes from a dog into a mouse allowing the production of antibody heavy chains that comprise a variable antibody region originating from the expression of canine DNA in the mouse, in combination with a constant region. The constant region may be the rodent immunoglobulin (IG) constant region, resulting in production of chimeric heavy chains having canine variable region and a rodent constant region. Information concerning, or the nucleic acid comprising, the variable region of such chimeric antibody chains may be used to generate fully canine antibodies, for therapeutic use in dogs for example. The rodent containing the canine DNA may also serve as an animal model for understanding of disease and testing of medicines.

For DNA immunisation, a prime and boost regime using hydrodynamic tail vein injection (HTVI) was performed and tissues harvested.

For cell-based immunisation, a prime and boost regime using MEF cells stably expressing OX40L was performed and tissues harvested.

A further immunisation regime was performed using HTVI DNA immunisation for the prime combined with OX40L-expressing MEF cell boosts.

### Determination of Serum Titres:

Mice were bled prior to immunisation and 10 days after each subsequent boost. Serum and red blood cells were separated using microvette 200 Z-gel tubes (Starstedt AG & Co. KG, Germany). Post-immunisation serum was serially diluted in FACS buffer (PBS+3% FBS) and added to either 10⁵ wild type cells or 10⁵ of the same cells stably-expressing OX40L. Mouse antibodies were detected with 1/200 dilution of BB700 conjugated 2° monoclonal antibodies against isotypes IgG1, IgG2a, IgG2b (BD OptiBuild™, Becton Dickinson). Stained cells were analysed by flow cytometry using a BD Accuri C6 Flow Cytometer (Becton Dickinson, NJ, USA) or Beckman Coulter's CytoFLEX S. Pre-immunisation serum was used as control (FIG. 4).

### Example 4: Isolation of OX40-L-Specific Antibody Producing Cells

### Tissue Isolation:

Spleens, lymph nodes and bone marrow were harvested from mice immunised with canine OX40L as described above. Splenocytes were prepared by cutting the spleen into pieces and forcing these through a 45 μm cell strainer (Falcon) while rinsing with RPMI-1640 (Lonza, Basel, CH)+10% FBS on ice. A similar process was used for lymphocytes from lymph nodes. Bone marrow was collected from femur and tibia and flushing the marrow with RPMI-1640 using a 21-gauge needle, through a 45 μm cell strainer pre-wetted with RPMI-1640. All cell types were pelleted at 300 g for 5 min, before either being directly used for flow sorting or resuspended in FBS+10% dimethyl sulfoxide (DMSO) and being frozen at −150° C.

### Cell Sorting:

Antigen-specific cells can be captured by fluorescent labelled VLPs or antigen protein probes. VLPs are generated from HEK cells stably transfected with OX40L, and subsequently transiently transfected with the retrovirus gag protein, and fluorescently labelled MA (gag matrix fragment p15-GFP fusion protein); the gag expression enables VLP budding from cells, and MA labels the VLPs for fluorescence-detection.

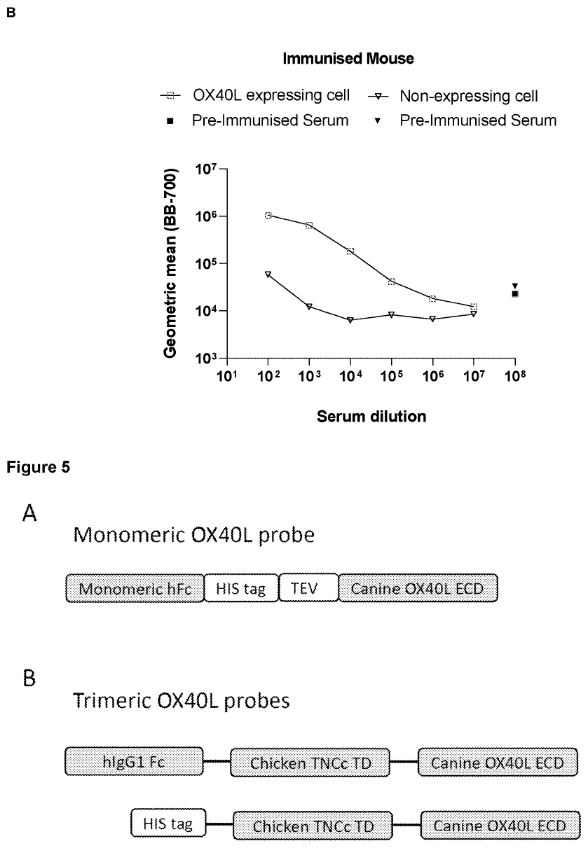

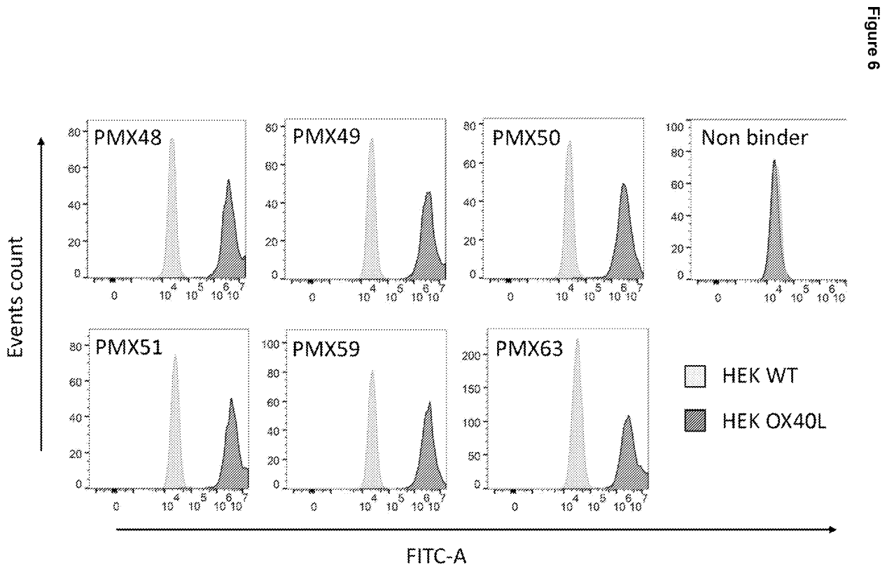

Antigen-specific B cells can also be captured by labelled antigen protein probes. Monomeric OX40L protein containing canine OX40L extra cellular domain (ECD), or trimeric soluble canine OX40L extra cellular domain probes (shown in the schematic in FIG. 5 and with reference to Willett et al. Mol Immunol. 2009, 46(6); 1020-1030) were synthesised in expression vectors and expressed in CHO cells. Fc tagged probes were purified from culture supernatant using Mab select SuRe protein A resin (Cytiva) or by AKTA using Mab Select SuRe columns (Cytiva). HIS tagged trimeric canine OX40L protein was purified using HIS-Pur Ni-NTA resin (ThermoFisher).

Conjugation of OX40L ECD TNCc HIS (MW=62.143 kDa; trimeric, Seq ID 811) to Alexa Fluor 647 was carried out using the Microscale Protein Labeling Kit (Molecular Probes—Invitrogen catalogue number A30009) according to the manufacturer's protocol. Degree of labelling was determined using a NanoDrop spectrophotometer.

Similarly, the conjugation of monomeric dOX40L-mvhfc (MW=40.435 kDa) (Seq ID 807) and canine OX40L ECD TNCc Fc (MW=280.980 kDa; hexameric, Seq ID 809) to Alexa Fluor 647 was carried out using the Alexa Fluor 647 Antibody labelling kit (Molecular Probes—Invitrogen catalog number A20186) following manufacturer's protocol for antibody labelling. Degree of labelling was determined using a NanoDrop spectrophotometer.

Probe specificity was determined on splenocytes from OX40L immunised or irrelevant antigen immunised mice. The optimal probe concentration was also determined based on maximum signal in relevant immunisation and minimally staining on irrelevant immunisation.

Sorted B cells are prepared using the 10XGenomics Chromium Single Cell Immune Profiling system and the V(D)J Kit (10XGenomics) according to the manufacturer's instructions. Nucleotide sequences of expressed antibodies are determined by Illumina MiSeq sequencing with 600 cycles (2×300 cycles) or by Ilumina iSeq, Miseq, MiniSeq, Nextseq, Hiseq 4000 or Novaseq sequencing with 2×150 cycles. The sequences are analysed using custom tools based on the pRESTO/Change-O (Yale University)/IgBlast (NCBI, USA) software to identify paired VH and VL sequences and a clonal lineage information is also constructed based on the identities of the heavy chain V, D, J and light chain V, J genes.

An example of the analysis of antibody sequences of sorted Ag-specific single B-cells is shown in FIG. 5 of WO2015/040401, and shows antibody sequences that are arranged by heavy-chain V-gene family usage, and clustered to generate the displayed phylogenetic trees. From phylogenetic trees such as these, candidate clones are selected. The nucleic acid and amino acid sequences of candidate clones VH and VL and their corresponding CDRs are given in the Sequences Table 2 below.

For instance, anti-canine OX40L mAbs PMX025-027 are all encoded by the same heavy chain V-gene (cIGHV3-5) and light chain V-gene (cIGLV3-14), anti-canine OX40L mAbs PMX039, 041, 043, 044, 046, 050 are all encoded by the same heavy chain V-gene (cIGHV3-18) and light chain V-gene (cIGLV3-3) (Table 3).

PMX025-065, PMX082-111, PMX154-156 and PMX291-294 have the sequences set out in Table 2 below.

### Example 5: Generation of Monoclonal Antibodies from Single Cells

In some examples, the heavy chain and light chain V(D)J sequence of selected candidate clones are synthesised and cloned into expression vectors containing the Fc regions, such as human IgG4, canine IgGB for the heavy chain, and human IgGK or canine IGK or IGL constant regions for the light chain. In some examples the IgGB constant region includes mutations that render the constant region effector deficient. Suitable sequences for these mutated Fc regions are given in the Sequence table below (Seq ID 723 to 730).

DNA encoding for canine heavy chain variable regions selected as described above are cloned in expression vectors upstream constant regions (CH1-hinge-CH2-CH3) of canine IgGB Def2 (SEQ ID NO:723). DNA encoding for canine light chain variable regions are cloned in expression vectors upstream constant regions of canine kappa or lambda light chains.

The expression vectors encoding the heavy chain and light chain are co-transfected into a suitable mammalian cell line such as CHO cells to obtain stable or transient expression.

For antibody production, 6×10⁶ selected stably transfected CHO cells are seeded in 3 ml culture media and incubated at 32° C., 5% CO₂ with shaking at 200 rpm. 4% HyClone Cell Boost 7a supplement+0.4% HyClone Cell Boost 7b supplement+1% glucose is added to the media on days 1, 4, 7 and 10. Culture supernatants are collected on day 12 and the IgG concentration determined on a protein A chip using surface plasmon resonance (Biacore 8K, Cytiva Life Sciences).

### Example 6: Binding Assays of OX40L Candidate Antibodies

### Binding Assays: Binding of OX40L Candidate Antibodies to Cell Surface Expressed Canine OX40L Proteins.

CHO supernatants diluted in FACS buffer (PBS containing 3% FBS) at 1, 5 or 10 μg/ml were used to screen for their binding capacity to canine OX40L using a cell-based assay. In brief, HEK293 or MEF cells (or any other commonly used cell lines) expressing canine OX40L at the cell surface are incubated with 100 μll of FACS buffer containing the candidate antibody for 30′ on ice. Cells are washed with 150 μl FACS buffer followed by centrifugation at 300 g for 3 min. The cell pellet is resuspended in FACS buffer containing a 1:1000 dilution of a fluorescently labelled secondary antibody recognising the canine Fc region of the test antibody for 30 min in the dark, followed by washing with 150 μl FACS buffer. Cells are resuspended in FACS buffer and flow cytometry performed using either a Cytoflex (Beckton Dickinson) or an Accuri (Beckman Coulter) cytometer, followed by data analysis using FlowJo (FIG. 6 and Table 4).

### Example 7: Functional Assays for OX40 and OX40L Activity of Candidate Antibodies

HEK-Blue—NF_{K}B functional assay:

### Cell-Based Signalling Inhibition Using HEK-Blue—NF_{K}B Assay:

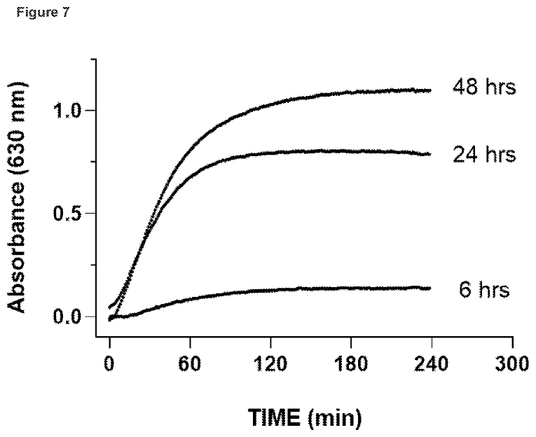

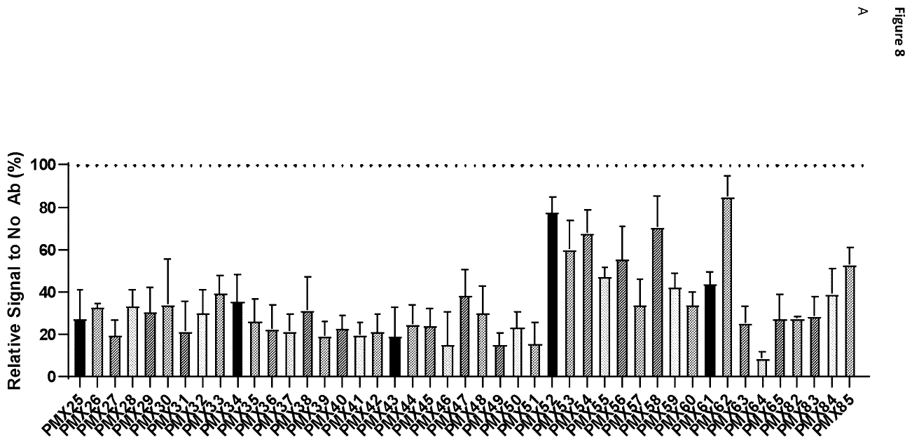

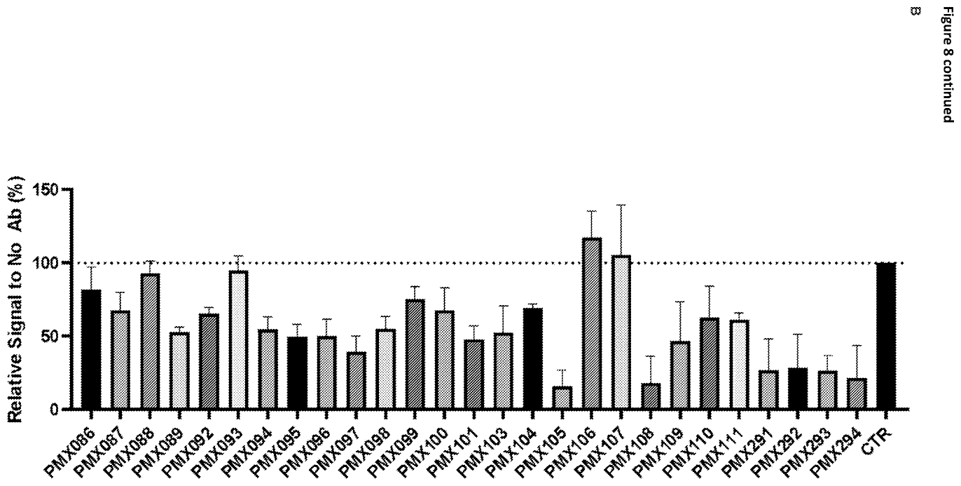

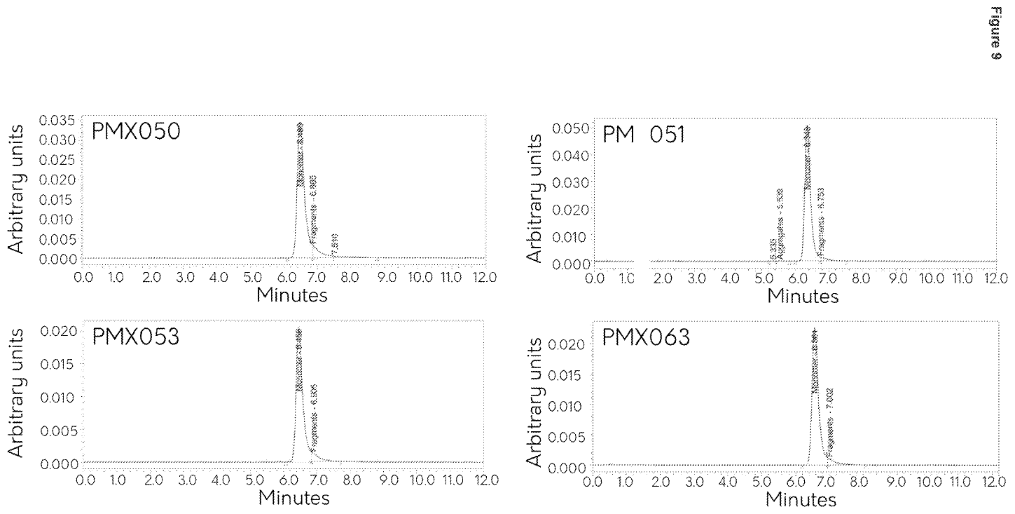

In order to quantify the signalling activation of OX40 and OX40L, a cell-based reporter assay was developed. Since OX40 signals via the NF_{K}B signalling pathway, canine OX40 (long form) (Seq ID 5 and 6) was stably transfected into HEK-Blue™ Null1-v cells (HEK blue OX40 cells). HEK-Blue™ Null1-v cells express a secreted embryonic alkaline phosphatase (SEAP) reporter gene under the control of the IFN-β minimal promoter fused to five NF-κB and AP-1 binding sites. Therefore, stimulation of canine OX40 expressing HEK-Blue™ Null1-v cells induces production and secretion of SEAP, the activity of which can be detected using the QuantiBlue detection kit (Invivogen, San Diego, USA). To provide the signalling molecule for canine OX40, canine OX40L (Seq ID 1 and 2) was transfected into wild type HEK cells (HEK OX40L cells). Co-culturing of HEK blue OX40 cells with HEK OX40L cells with 1:1 ratio results in NF_{K}B signalling activation (FIG. 7).

Prior to the assay, candidate antibodies were batch purified from CHO supernatant using protein A resin (MabSelect SuRe LX, Cytiva). In brief, MabSelect SuRe LX resin is washed in PBS and diluted to a 10% slurry. An appropriate volume of the 10% slurry is added to the supernatant and incubated 5-10′ at RT before being loaded into a filter column. After 3 washes with PBS, the antibodies are eluted using IgG elute buffer (Pierce) directly into neutralisation buffer (10 mM TRIS pH8.0) with 8:1 IgG elute: neutralisation buffer ratio. Antibody concentrations are quantified by measuring absorbance at 280 nm, using Nanodrop One (Thermo Fisher).

To determine the inhibitory effect of candidate antibodies on OX40L-OX40 signalling, 5,000 HEK-blue-OX40 cells and 5,000 HEK-OX40L cells are seeded in a 96 well plate in presence of protein A purified anti-OX40L antibodies at a fixed concentration of 6.6 nM. After 24 h incubation at 37° C., 20 μl of supernatant is collected and the presence of SEAP is assessed by the addition of 180 μl of QuantiBlue reagent and a further 4 hr incubation at 37° C., followed by measuring absorbance at 630 nm with a ClarioStar plate reader (BMG Labtech). Data is analysed using the dedicated MARS software version 3.40 R2 (BMG Labtech) and GraphPad Prism version 9.1. Data are shown in FIG. 8 and Table 5.

### Ex Vivo PBMC Activation Assay.

PBMCs are isolated from freshly drawn canine whole blood, with sodium heparin anticoagulant, using Ficoll-Paque plus (Cytiva, GE17-1440-02) density gradient centrifugation. In brief, canine blood is diluted 1:1 with phosphate buffer saline (PBS) and carefully layered on top of Ficoll-paque plus, centrifugated at 800 rcf for 20′ with slow acceleration and no break at the end. The top layer and interphase disk are diluted with PBS and centrifuged at 1300 rpm for 10′ to collect PBMCs. Pelleted PBMC is washed for a second time with PBS to remove all remnants of Ficoll. After a second centrifugation, PBMCs are resuspended in media (PBMC media=RPMI+10% heat inactivated foetal bovine serum+1% penicillin-streptomycin+1% non-essential amino acids+1% L-glutamine+1% sodium pyruvate+1% HEPES) before use or freezing.

Freshly isolated or thawed PBMCs are seeded in a 96 well plate in 200 μl/well of PBMC media supplemented with PHA (Sigma Aldrich) or ConA (ThermoFisher) and cultured with or without purified anti-OX40L antibodies at specified concentrations at 37° C., 5% CO₂. Supernatant is then collected at day 3-5 for cytokine quantification. IFN-γ is measured by ELISA (MABtech) according to manufacturer's recommendations.

Alternatively, freshly isolated or thawed PBMCs are seeded in a 96 well plate precoated with a combination of anti-canine CD3 (Biorad) and CD28 (eBioscience) antibodies and cultured with or without additional canine OX40L trimeric protein or HEK cells expressing canine OX40L, and/or purified anti-OX40L antibodies at different concentrations at 37° C., 5% CO₂. Supernatant is then collected at day 3-5 for cytokines quantification. IFN-γ is measured by ELISA (MABtech) according to manufacturer's recommendations.

Table 6 and 7 show raw and normalised IFN-γ levels obtained from PBMCs cultured for 3 days in a 96 well plate (1 to 2×10\hat{ }⁵ PBMC per well) precoated with a combination of anti-canine CD3 (Biorad) and CD28 (eBioscience) antibodies and cultured with 5000 HEK cells expressing canine OX40L (or 5000 wild type HEK cells as control), and the indicated anti-OX40L antibodies at 66, 6.6 or 0.66 nM. Table 6 show the calculated IFN-γ levels in supernatant. As levels of secreted IFN-γ varies greatly between experiments due to the usage of different PBMC donors, we normalised the level of secreted IFN-γ in wells with PBMC activated with CD3-CD28 in presence of HEK cells expressing OX40L and anti-OX40L antibodies for the level of secreted IFN-γ in wells with PBMCs activated with CD3-CD28 (0%) and PBMC activated with CD3-CD28 in presence of HEK cells expressing OX40L but no anti-OX40L antibodies (100%) in the same experiment (Table 7).

### Example 8: Affinity Determination of Anti-OX40L Antibodies Candidates

### Determination of Antibody Affinity by Surface Plasmon Resonance (SPR)

Affinity (K_{d}) of OX40L antibodies is measured by SPR, using a recombinant canine OX40L ECD-TNCc-HIS protein (Seq ID 811) as the capturing agent (FIG. 5B).

OX40L ECD-TNCc-HIS protein is purified from CHO supernatant using HIS-Pur Ni NTA resin (ThermoFisher). In brief, CHO supernatant is buffer exchanged into PBS pH 8.0 containing 10 mM imidazole, using PD10 desalting columns (Cytiva). The buffer exchanged supernatant is then incubated 1 h at 4° C. with HIS-Pur Ni NTA resin in rotation. Resin is applied to a gravity filter column and washed 3 times with PBS pH 8.0 containing 10 mM imidazole before elution with 2.5 ml of PBS pH 8.0 containing 500 mM imidazole and buffer exchange to PBS pH7.4 using PD10 desalting columns.

Functional affinity is determined using a Biakore 8K (Cytiva). in brief, purified OX40L ECD-TNCc-HIS protein is covalently bound at low density to the surface of a CM5 sensor chip (Cytiva) by amine coupling, using the manufacturer's recommended protocol. Candidate antibodies are subsequently passed across the chip surface at a range of concentrations and affinity determined using the dedicated software (Biacore Insight Evaluation Software). Curves were fitted using a bivalent analyte binding model. Results are summarised in Table 8.

### Example 9: Determination of Candidate Anti-OX40L Antibodies Stability

After protein A purification, anti-OX40L antibodies were eluted using IgG elution buffer (Pierce) and buffer acidity was neutralised using 1 M TRIS pH 8.0. Candidates' stability was assessed in such buffer or after buffer exchange into sodium acetate buffer pH 5.5 (20 mM Sodium acetate 100 mM NaCl pH 5.5).

The tendency of candidates to form aggregates or fragments was first assessed by high performance liquid chromatography-size exclusion chromatography (HPLC-SEC). In brief, HPLC-SEC chromatography (column: BioResolve SEC mAb 200A, 2.5 um column WATERS) was performed using ACQUITY H-class Bio (WATERS) using PBS as mobile phase with isocratic 0.575 mL/min flow rate. 10 μL of each sample have been injected in H-SEC using the above protocol. Percentage of monomeric species and Area (indicative of antibody concentration) was determined for each molecule by manual integration on the chromatogram (FIG. 9 and Table 9).

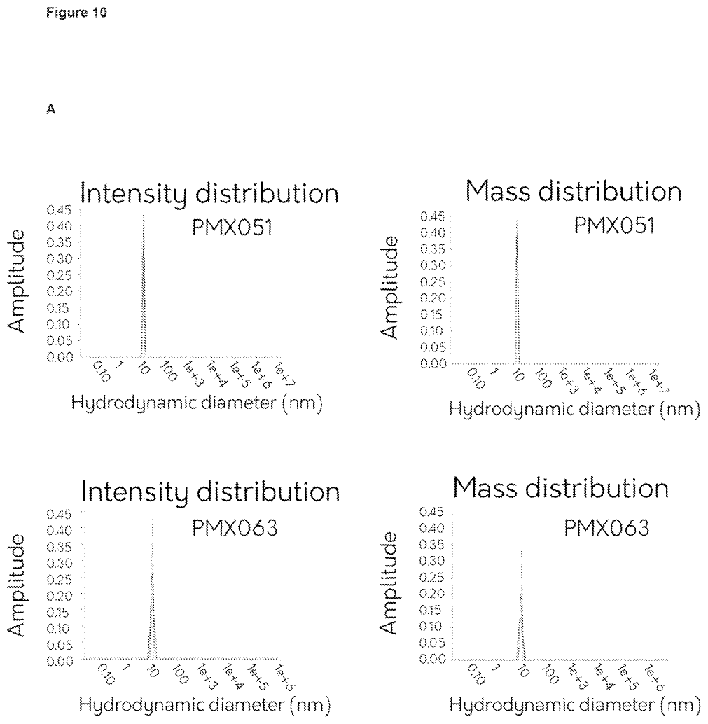

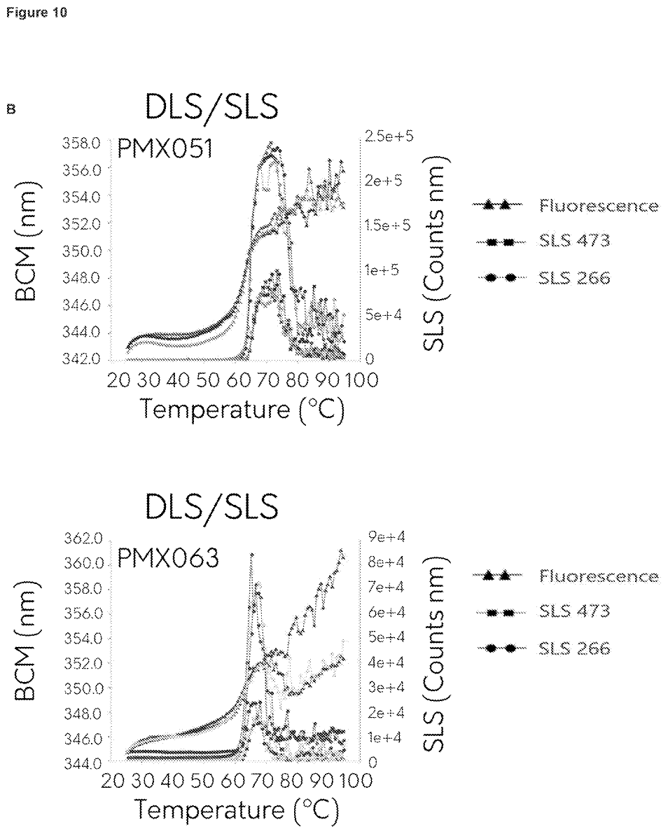

Candidate stability, fragmentation and aggregation propensity was further assessed by evaluating melting temperature (Tm1), aggregation temperature (Tagg266) and initial diameter by static light scattering (SLS) and dynamic light scattering (DLS) using UNcle (Unchained lab). In brief, 3×9 μL of each sample was loaded into the Uni. The data from individual experiments were analysed and outliers discarded, and the remaining data averaged. Thermal ramp stability measurements were taken over 25 to 95° C. at a ramp rate of 0.3° C./min with an incubation period of 120 s, while 10 DLS measurements per sample was recorded at the start and the finish. DLS measurements (size distribution and polydispersity) were calculated with the UNcle software (version 5.03) correlation function. For SLS the intensity of scattered light was measured at 266 nm and was used to calculate the aggregation temperature (Tagg) with UNcle software (version 5.03). The intrinsic fluorescence emission was measured at 473 nm and the barycentric mean (BCM) was used to calculate the melting temperature (Tm) using the same software. Data are summarised in Table 9 and an example of the profile showing PMX051 and PMX063 are shown in FIG. 10.

### Example 10: Sequence Analysis and Candidate Clustering

To expand on existing identified candidates with desirable properties, we searched paired B cell receptor sequences derived from canine OX40L immunisation. Given a query CDR3H peptide, we searched for candidate sequences with a CDR3H of the same length but allowing up to two amino acid variations. Using PMX051 and PMX063 as the lead sequence, 26 new antibodies (PMX086-PMX111) are identified (Table 2).

The 78 anti-OX40L antibodies were subsequently sub-grouped into 14 family groups by V-gene pair.

### Example 11 Fc Variants

### Fc Variants Construction

For antibody production, DNA constructs were generated to encode chimeric antibodies comprising selected canine IgG constant regions fused to the variable regions of anti-human CD20 antibodies, Rituximab (see U.S. Pat. No. 5,736,137) or Ofatumumab (sequence available on Drugbank https://go.drugbank.com/drugs/DB06650).

The canine IgG-B mutant variants (Def 1 to 9, Seq ID 722 to 730) were generated by site directed mutagenesis. Specifically, human Ofatumumab or Rituximab variable region and canine IgG-B mutant variants are PCR amplified using Q5 high fidelity DNA polymerase and assembled into a mammalian expression vector upstream sequences encoding for either canine IgGB WT or Def 1 to 9 (Seq ID 721 to 730) using NEBuilder HIFI DNA Assembly (New England Biolabs). Ofa-VH-cIgGB WT Ofatumumab sequence is shown in SEQ ID NO. 731. In the expression vector, the heavy chain and the antibiotic resistant gene expression units are flanked by DNA transposon piggyBac terminal inverted repeats to mediate stable integration into host cells in the presence of piggyBac transposase. The expression vectors encoding the heavy chain and light chain are co-transfected into a suitable mammalian cell line such as CHO cells together with PiggyBac transposase to obtain stable expression. For antibody production, 3×10⁶ selected CHO cells are seeded in 3 ml culture media and incubated at 32° C., 5% CO₂ with shaking at 200 rpm. 4% HyClone Cell Boost 7a supplement+0.4% HyClone Cell Boost 7b supplement+1% glucose is added to the media on days 1, 4, 7 and 10. Culture supernatants are collected on day 12 and the IgG concentration determined using surface plasmon resonance using protein A chip (Biacore 8K, Cytiva Life Sciences).

### Complement Dependent Cytotoxicity (CDC) Activity

Canine lymphoid tumour cell lines such as CLBL1 (University of Veterinary Medicine Vienna) or CLC (Umeki S. et al J. Vet. Med. Sci., 75:467-474 (2013) PubMed=23196801; DOI=10.1292/jvms.12-0448) may be used as target cells. These cells are stably transfected or nucleofected with an expression construct encoding for the human CD20 protein (Seq ID 732) to generate hCD20-expressing cells. The expression constructs were generated using the same method as described above for antibody expression.

Transfected cells were selected for puromycin resistance and hCD20^{high} (top 5%) cells were FACS sorted by staining for hCD20 expression using anti-human CD20 antibody (clone: 2H7, BioLegend).

To assess CDC activity, untransfected (wild type) target lymphoid cells or equivalent hCD20-expressing cells were used in a cell killing assay in which 5000 target cells per well of 96-well plate were incubated with anti-human CD20 canine Fc chimeric antibody as described above and canine complement preserved serum (BioIVT) at a final dilution of 1:12, for 2 hours at 37° C., 5% CO₂. The assay was set up using media (RPMI+1% L-glutamine+20% fetal bovine serum for CLBL-1 cells made using heat inactivated serum so that canine complement preserved serum would be the only source of complement.

Live cells were then quantified using CellTitre-Glo® Luminescent Cell Viability Assay (Promega) following the assay protocol. This assay uses the ATP content of live cells as an indication of cell viability. Luminescence was measured on a CLARIOstar (BMG Labtech). Data was analyzed using MARS software (BMG Labtech) and the number of live cells remaining was used to calculate the percentage of killing in the presence of antibodies using Microsoft Excel. Background signal was obtained from a sample of cells treated with 1% triton (where no cells were left alive) and subtracted from the signal obtained from each test samples. Max signal (0% killing) was obtained from a sample of cells treated identically but where antibodies were omitted. Graphs were plotted in Microsoft Excel or GraphPad Prism. Exemplary CDC assays were performed using hCD20 expressing CLBL1 cells and WT CLBL1 cells and a titration of IgGB WT or Def mutant antibodies with Ofatumumab variable regions. Antibodies were used at serial 1:3 dilutions ranging from 10 μg/ml to 0.01 10 μg/ml.

All Def mutants tested (Def1, 2, 3, 5, 6, 7, 8, 9) completely abrogated the ability of canine IgGB WT to kill hCD20 CLBL1 cells by complement dependent cytotoxicity (data not shown).

### Antibody Dependent Cellular Cytotoxicity (ADCC) Activity

Canine cell lines, such as MDCK II cells (ATCC) were stably transfected or nucleofected with a construct encoding for the human CD20 protein and a construct expressing a fluorescent protein (i.e. GFP), as described above. Wild type MDCK II cells expressing GFP were used as a control. Canine peripheral blood mononuclear cells (PBMCs, Envigo) were used as a source of effector cells. PBMCs were isolated from freshly drawn whole blood, with sodium heparin anticoagulant, using Ficoll-Paque plus (Cytiva, GE17-1440-02) density gradient centrifugation. In brief, 10 ml blood was diluted 1:1 with phosphate buffer saline (PBS) and carefully layered on top of 15 ml of Ficoll-paque plus, centrifugated at 800 rcf for 20′ with slow acceleration and no break at the end. The top layer and interphase disk were diluted with PBS and centrifuged at 1300 rpm for 10′ to collect PBMC in the pellet which was washed a second time in PBS to remove all remnants of Ficoll. After a second centrifugation, PBMCs were resuspended in media (PBMC media=RPMI+10% heat inactivated foetal bovine serum+1% penicillin-streptomycin+1% non-essential amino acids+1% L-glutamine+1% sodium pyruvate+1% HEPES) supplemented with 50 ng/ml recombinant canine IL-2 (R&D systems) and incubated for 24 h at 37° C., 5% CO₂ before use in the ADCC assay.

To assess ADCC activity of Def mutants antibodies, MDCK II cells wild type or expressing hCD20, both expressing GFP, were co-cultured with PBMCs at an effector: target ratio of 50:1 or 100:1 and a titration of IgGB WT or Def mutant antibodies with Ofatumumab variable regions for 24 h at 37° C., 5% CO₂, in a 1:1 mix of MDCK II media (DMEM+1% L-glutamine+10% fetal bovine serum) and PBMC media.

GFP signal, which is proportional to the number of live cells per well, was used as a measure of the number of live cells remaining in the well at the end of the 24 h incubation. GFP signal was measured on a CLARIOstar (BMG Labtech). Data was analysed using MARS (BMG Labtech) and percentage of killing in the presence of antibodies was calculated using Microsoft Excel. Background signal was obtained from a sample of cells treated with 1% triton (where no cells were left alive) and subtracted from the signal obtained from each test samples. Max signal (0% killing) was obtained from a sample of cells treated identically but where antibodies were omitted. Graphs were plotted in Microsoft Excel or GraphPad Prism.

Def mutants 2, 3 and 7 were the best at reducing canine IgGB ADCC activity and strongly reduced canine IgGB ADCC activity at up to 10 μg/ml (data not shown).

### Example 12 Long Term Stability and Thermal Stability

After protein A purification, anti-OX40L antibodies were eluted using IgG elution buffer (Pierce) and buffer acidity was neutralised using 1 M TRIS pH 8.0. Candidates' stability was assessed in such buffer or after buffer exchange into sodium acetate buffer pH 5.5 or glycine buffer. After buffer exchange the candidate antibodies were concentrated to 5 mg/ml before proceeding with analysis or incubation at 4° C. or 37° C.

For long term stability, candidate antibodies were incubated at 4° C. for up to 4 months. For thermal stability assessment, candidate antibodies were incubated at 37° C. for up to 28 days.

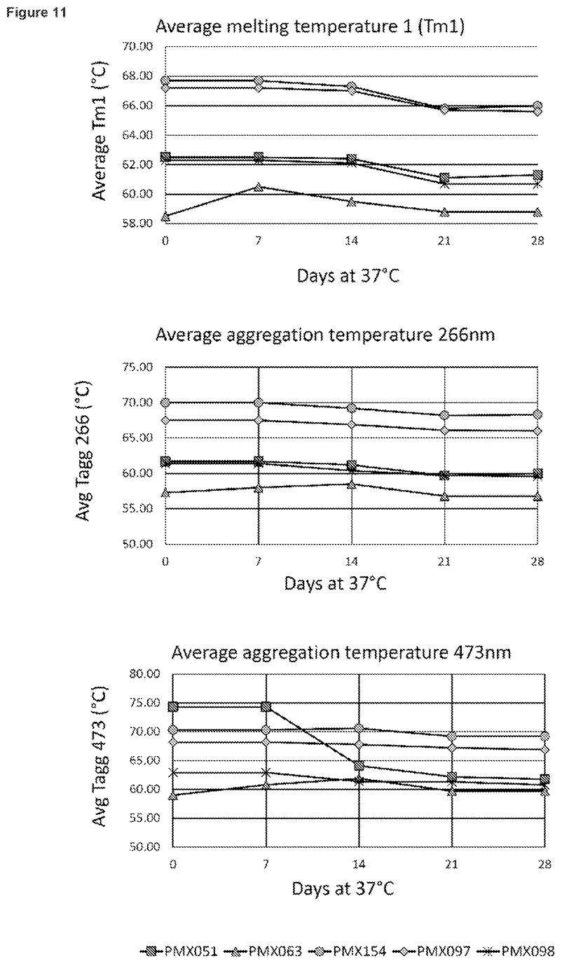

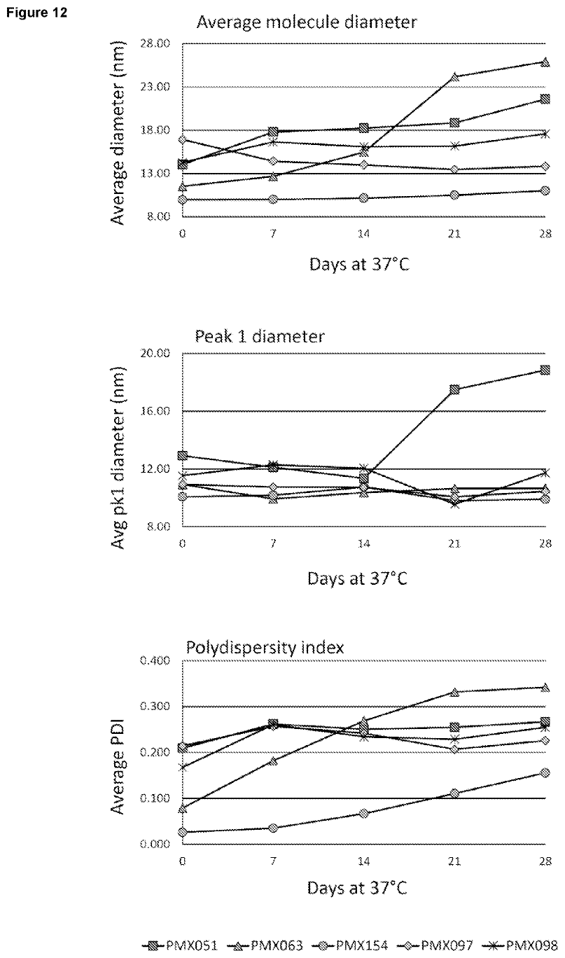

Candidate stability, denaturation and aggregation propensity was further assessed by evaluating melting temperature (Tm1), aggregation temperature (Tagg 266 and Tagg 473) and initial diameter by static light scattering (SLS) and dynamic light scattering (DLS) using UNcle (Unchained lab). In brief, 3×9 μL of each sample was loaded into the Uni. The data from individual experiments were analysed and outliers discarded, and the remaining data averaged. Thermal ramp stability measurements were taken over 25 to 95° C. at a ramp rate of 0.3° C./min with an incubation period of 120 s, while 10 DLS measurements per sample was recorded at the start and the finish. DLS measurements (size distribution and polydispersity) were calculated with the UNcle software (version 5.03) correlation function. For SLS the intensity of scattered light was measured at 266 nm or 473 nm and was used to calculate the aggregation temperature (Tagg 266 and Tagg 473, respectively) with UNcle software (version 5.03). The intrinsic fluorescence emission was measured at 473 nm and the barycentric mean (BCM) was used to calculate the melting temperature (Tm) using the same software. Data for thermal stability at 37° C. for PMX051, PMX063, PMX097, PMX098 and PMX154 are shown in FIGS. 11 and 12. Data for long term stability at 4° C. for PMX097 and PMX154 are summarised in Table 10.

### Example 13 Pharmacokinetic Profile

To determine the pharmacokinetic profile of anti-OX40L antibodies, laboratory beagle dogs were injected intravenously with 3.0 mg/kg of either PMX097 or PMX154. Blood samples were collected the day before injection and after 2 hours, 6 hours, 24 hours, 48 hours, 4 days, 8 days, 15 days, 21 days, 29 days, and 35 days. After coagulation, blood was centrifuged at 7000 g for 5 minutes to separate the serum which was then stored at −80° C.

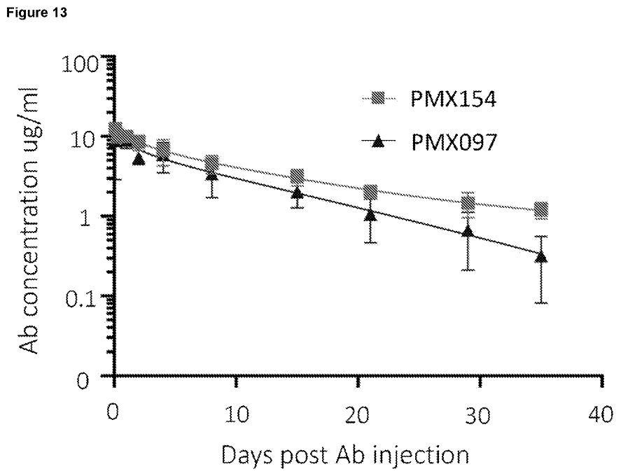

After collection of all time points the pharmacokinetic profile was determined by enzyme-linked immunosorbent assay (ELISA). Briefly, ELISA plates were coated overnight at 4° C. with HIS tagged trimeric OX40L protein (0.2 μg/ml in PBS, Seq ID 811). The next day plates are blocked for 2 hours with blocking buffer (PBS containing 0.05% tween 20 and 1% bovine serum albumin fraction V (BSA)) before washing 5 times with PBS containing 0.05% tween 20. Before application, sera are diluted 1 in 2000 in blocking buffer. Serial dilutions (one in two dilutions) of PMX097 or PMX154 were prepared in canine serum from a top concentration of 50 μg/ml. The standard dilutions were also diluted 1 in 2000 in blocking buffer before applying to the ELISA plates. After incubating sera and standard for 2 hours the plates were washed 5 times and incubated 1 hour with a secondary antibody anti dog IgG conjugated with biotin (Merck) diluted 1 in 100.000 in blocking buffer. After 5 more washes plates are incubated with streptavidin-HRP (Biolegends) diluted 1 in 5000 in blocking buffer, then washed again before adding TMB substrate (Pierce) and stopping the reaction with 0.2N sulphuric acid right before data acquisition. Absorbance at 450 nm and 650 nm was quantified using a plate reader (CLARIOstar, BMB Labtech). Data were analysed using the integrated MARS software (BMG Labtech). Concentration of samples in serum is determined by the MARS software using the standard curve generated from the serial dilution of PMX097 or PMX154, plotted as a four-parameter logistic curve fitting algorithm. Data shown in FIG. 13 were plotted using Prism and the half-life was calculated fitting the points with a two-phase decay curve fitting.

### Example 14 Pharmacodynamic Profile

The ability of PMX097 and PMX157 to suppress T cell activation in vivo was assessed in laboratory beagle dogs. To induce T cell activation, we immunised the dogs with keyhole limpet hemocyanin (KLH). The schedule of the procedures is listed in table 11. Briefly, 20 dogs were divided in five groups that received a different treatment regime (Table 11). A single dose of 3 mg/kg or 0.5 mg/kg of PMX097 or PMX154, or vehicle control, was infused intravenously on day 0. The day following antibodies injection, all dogs received a first KLH injection. A second, intradermal, KLH injection was performed 3 weeks later and, two days after that, two full thickness skin biopsies (punch biopsies) were collected: one at the KLH injection site and one in a non-injected area, as control.

For serum preparation, blood samples were collected the day before injection and after 2 hours, 6 hours, 24 hours, 48 hours, 4 days, 8 days, 15 days, 21 days, 29 days, and 35 days. After coagulation, blood was centrifuged at 7000 g for 5 minutes to separate the serum which was then frozen. For PBMC isolation, whole blood was collected in tubes containing sodium heparin the day before antibody injection and after 8 days, 15 days, 21 days, 29 days, and 35 days. PBMC were isolated from whole blood on the same day of collection using lymphocyte separation medium (Corning) and frozen right after for long term storage in liquid nitrogen. In brief, canine blood is diluted 1:1 with phosphate buffer saline (PBS) and carefully layered on top of lymphocyte separation medium, centrifugated at 800 rcf for 20′ with slow acceleration and no break at the end. The top layer and interphase disk are diluted with PBS and centrifuged at 400 rcf for 10′ to collect PBMCs. Pelleted PBMC is washed for a second time with PBS to remove all remnants of lymphocyte separation medium. After a second centrifugation, PBMCs are resuspended in media (PBMC media=RPMI+10% heat inactivated foetal bovine serum+1% penicillin-streptomycin+1% non-essential amino acids+1% L-glutamine+1% sodium pyruvate+1% HEPES) and diluted 1 to 1 with cold freezing medium (80% heat inactivated FBS, 20% DMSO) before freezing.

### Determination of T Cell Activation by Interferon Gamma ELISpot

The number of activated T cells in response to KLH stimulation was determined by interferon gamma (IFNY) enzyme-linked immunosorbent spot (ELISpot) sing the canine IFNY ELISpot flex ALP kit (Mabtech).

Frozen PBMC were thawed and rested to eliminate dying cells by culturing in PBMC medium in low adhesion polypropylene tubes in a humidified incubator at 37° C. with 5% CO₂ for between 18 hours and 48 hours. The day before setting up the experiment, ELISpot plates (Millipore) were activated by adding 15 μl of 35% ethanol per well, immediately followed by 5 washes with sterile distilled water. After activation plates were coated by incubating overnight at 4° C. with anti-canine IFNY antibody (MT13) diluted to 15 μg/ml in PBS. The next day plates were washed 5 times with PBS and blocked for at least 30 minutes with PBMC medium. After blocking 50 μl of PBMC medium alone (for no stimulation control) or containing KLH (Merk) at 100 μg/ml (2×) was added to each well. As positive control, 50 μl of PBMC medium containing Phytohemagglutinine-L (PHA) (sigma-Aldrich) at 20 μg/ml (2×) was added to the correspondent wells.

Before seeding onto ELISpot plates, PBMC were counted using an automated CellDrop (DeNovix) cell counter with primary AO/PI settings. Briefly, PBMC were resuspended and 10 μl of PBMC were mixed with 10 μl of acridine orange/propidium iodine (AO/PI) working solution to discriminate between live and dead cells before loading onto a CellDrop automated cell counter. After centrifugation to remove dead cells and debris, PBMC were then resuspended at 4.4×10\hat{ }6 cells per ml of PBMC medium and 50 μl of the cell suspension was added to the plates on top of the stimuli and incubated in a humidified incubator at 37° C. with 5% CO₂ for 20 hours.

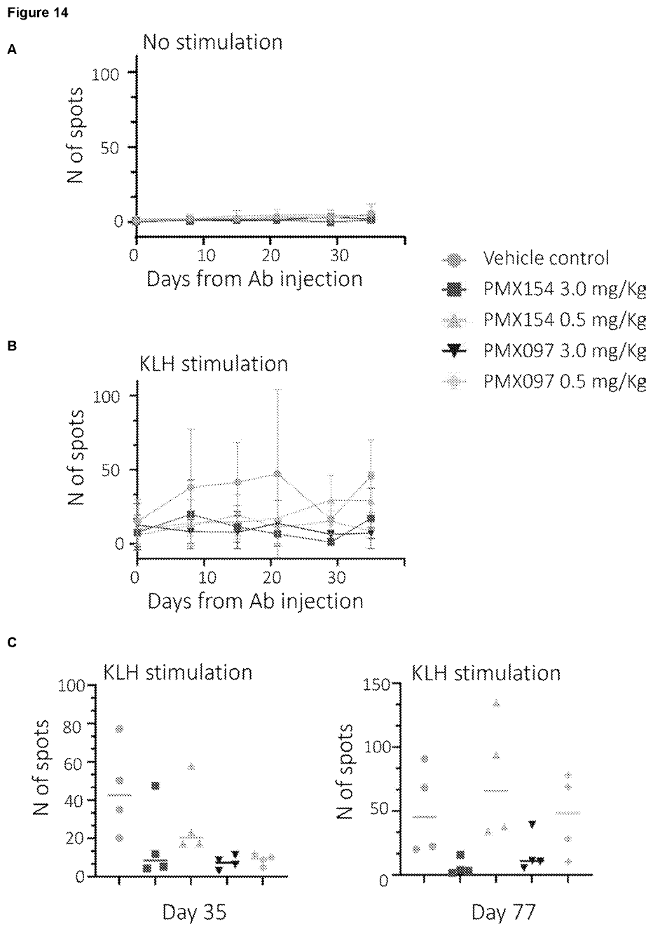

The next days the cells are discarded, and plates washed 5 times with PBS before incubating 1 hour with the detection antibody (MT166-biotin) diluted to 0.5 μg/ml in PBS with 0.5% FBS. Following 5 more washes with PBS, plates are then incubated 1 hours with streptavidin-ALP diluted 1 in 1000 in PBS with 0.5% FBS and washed another 5 times with PBS before adding BCIP/NBT plus substrate (Mabtech) till the spots become apparent. After stopping the reaction by washing with water, plates were air dried. Spots were quantified using an ELISpot counter (Immunospot). After quality control using the ELISpot counter integrated software, data were plotted using Prism. Data are presented in FIG. 14 and show a strong reduction of the number of KLH-responsive circulating T cells in dogs treated with PMX097 and PMX154 at all time points analysed. These data demonstrate the ability of both PMX097 and PMX154 to suppress T cell activation in vivo for up to 35 days, both when administered at 3.0 mg/ml and 0.5 mg/ml.

### Determination of Anti-KLH IgM and IgG Serum Titre

B cell activation and antibody secretion is also dependent upon helper T cells stimulation. Therefore, a reduced T cell activation should translate in a reduce humoral response. To evaluate the ability of PMX097 and PMX154 to reduce T cell-dependent antibody response (TCDAR) following KLH immunisation (Kawai R., Aida T. et al, The Journal of Toxicological Sciences, Vol. 38, No. 4, 571-579, 2013; Saghari M, Gal P, et al, Clinical Pharmacology & Therapeutics, Vol 111, N 5, May 2022), anti-KLH IgM and IgG serum titre was quantified by ELISA.

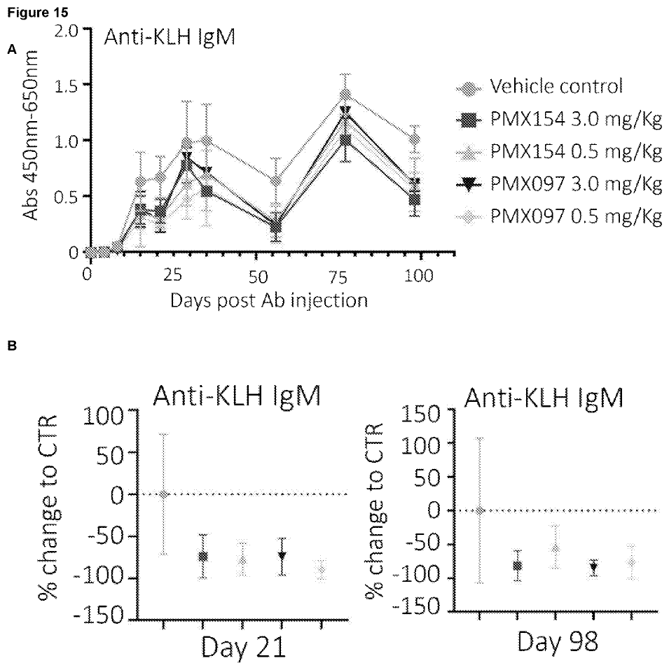

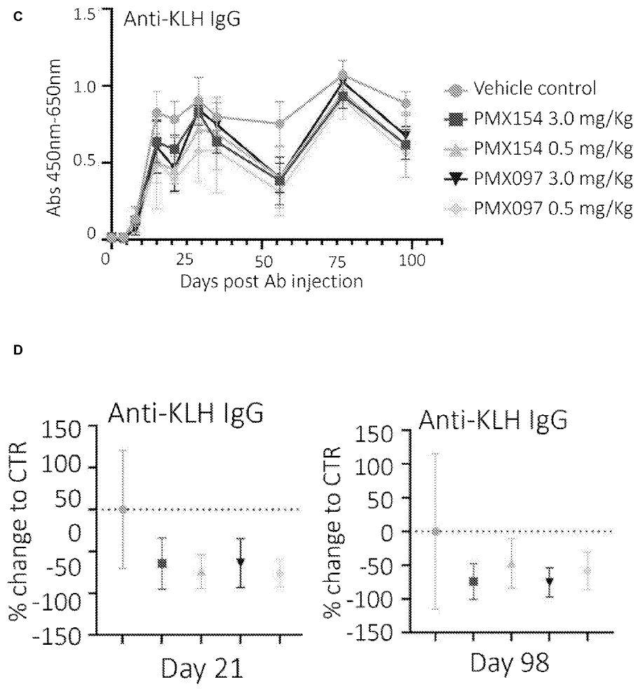

ELISA plates were coated overnight at 4° C. with KLH protein diluted to 1 μg/ml in PBS. The next day plates are blocked for 2 hours with blocking buffer (PBS containing 0.05% tween 20 and 1% bovine serum albumin fraction V (BSA)) before washing 5 times with PBS containing 0.05% tween 20. Before application sera are diluted 1 in 6000 or 1 in 15000 in blocking buffer, for anti-KLH IgM or IgG detection, respectively. After incubating sera and standard for 2 hours the plates were washed 5 times and incubated 1 hour with a secondary antibody: anti dog IgG conjugated with biotin (Merck) or goat anti dog IgM diluted 1 in 5000 or 1 in 500 in blocking buffer, respectively. After 5 more washes plates are incubated with either streptavidin-HRP (Biolegends) or an anti-goat HRP conjugated antibody diluted 1 in 5000 or 1 in 10000 in blocking buffer, respectively, then washed again before adding TMB substrate (Pierce) and stopping the reaction with 0.2N sulphuric acid right before data acquisition. Absorbance at 450 nm and 650 nm was quantified using a plate reader (CLARIOstar, BMB Labtech). Data were analysed using the integrated MARS software (BMG Labtech). Due to the unavailability of a standard, data are presented as blank corrected difference of absorbance (Abs) at 450 nm minus blank corrected Abs at 650 nm (FIG. 15 A and C) and as normalised to vehicle control treated samples (FIG. 14 B and D). Data shown in FIG. 15 were plotted using Prism and show the ability of both PMX097 and PMX154 to reduce both anti-KLH IgM and IgG serum titre for up to 98 days post administration.

### Effect of PMX097 and PMX154 on Local Inflammation in the Skin.

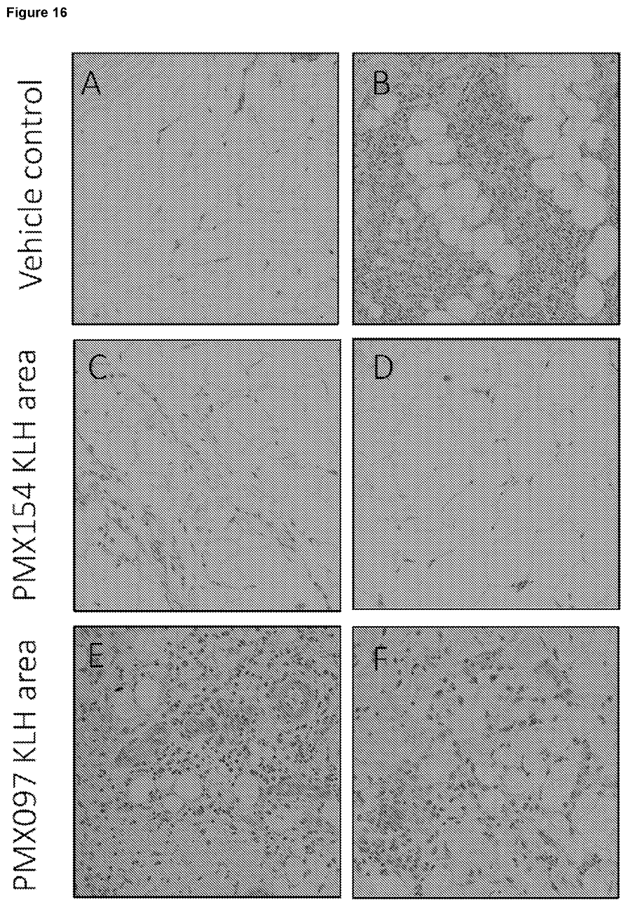

To evaluate the ability of PMX097 and PMX154 to reduce local inflammation in the skin, the second KLH immunisation was administered intradermally. This allowed collection of skin biopsies and histological analysis of the injection site. As control each dog also produced a skin biopsy from a non-injected site. Skin biopsies were fixed overnight in 10% normal buffered formalin and then moved into 70% ethanol the next day. After complete dehydration through incubation in ethanol 95% and then ethanol absolute for 1 hour each, biopsies were moved into xylene and then embedded in paraffin before cutting into 5 μm thick sections. Skin sections were then stained with haematoxylin and eosin and imaged using a wide field microscope (Nikon). FIG. 16 shows representative images that illustrate the severe reduction in immune infiltrates in the subcutaneous fat near KLH injection sites of dogs treated with PMX097 and PMX154.

To obtain an objective assessment of the phenotype, we next submitted haematoxylin and eosin-stained slides to histopathological evaluation. Microscopic changes were graded as to severity utilizing a standard grading system whereby 0=no significant change, 1=minimal, 2=mild, 3=moderate, and 4=marked. International Harmonization of Nomenclature and Diagnostic (INHAND) Criteria standards are used as the basis of evaluation—https://www.toxpath.org/inhand.asp. All tissue sections contained portions of epidermis and dermis, but deeper subcutaneous adipose tissue and muscular layer are variably present. When the subcutaneous adipose tissue or muscular layer were absent in a tissue section, the findings associated with those layers were excluded from analysis and represented by a “-” symbol in the tables. Use of numerical grades allows a mechanism to calculate a total score lesion score which can be used to assess prevalence and severity of tissue changes within and between groups. Results of histopathological analysis are summarised in Table 12 and 13.

Treatment with PMX097 and PMX154 at both 3.0 mg/kg and 0.5 mg/kg reduced the severity of immune infiltrates in KLH injected areas.

### Example 15 Safety Profile of PMX097 and PMX154 in Laboratory Beagle Dogs

The safety profile of PMX097 and PMX154 was assessed in laboratory beagle dogs, after intravenous injection of the two compounds at either 3.0 mg/kg or 0.5 mg/kg (Table 11). During and after antibody injection dogs were regularly monitored. To determine the effect of PMX097 and PMX154 on the animal health, complete blood chemistry and blood cellular count was performed on each dog at 3 time points: right before (pre-bleed), 24 hours and 15 days after antibody injection.

Regular monitoring did not reveal any abnormality. All animals in all groups tolerated well the administration and did not show any visible side effects for the entire duration of the study. Blood cell count and biochemistry parameters were mostly within range at all time points and there was no indication of toxicity in all treatment groups.

TABLE 2
SEQUENCES All sequence for antibody and antibody fragments designated as “PMX” followed
by a number and shown below are within the scope of the invention.

| SEQ | | |
|---|---|---|
| ID | | |
| NO: | Description | Sequence |
| 1 | Wild type canine | MEGVQPLDQNVGNTPGRRFQKNKVLLVAAIIQGLGLLLCFTYI |
| | OX40L amino acid | CLHFYASQVPPQYPPIQSIRVQFTRCENEKGCIITSPSKDETM |
| | | KVQDNSIIINCDGFYLISLKGYFSEELSLSLYYRKGRGPLFSLS |
| | | KVTSVDSIGVAYLAFKDKVYFNVTTHSTSYKDIQVNGGELILIH |
| | | QNPGGFCAY |
| 2 | Wild type canine | ATGGAAGGGGTCCAACCCCTGGACCAGAATGTGGGAAACA |
| | OX40L nucleic acid | CACCAGGGCGAAGATTCCAGAAGAACAAGGTATTGCTGGT |
| | | GGCAGCCATAATTCAGGGGCTGGGTCTGCTCCTGTGTTTC |
| | | ACCTACATCTGCCTGCACTTCTATGCTTCTCAGGTGCCGCC |
| | | TCAGTATCCTCCAATTCAAAGTATCAGAGTACAATTTACCA |
| | | GGTGTGAGAATGAGAAAGGTTGCATCATCACATCCCCAAG |
| | | CAAGGATGAAACTATGAAGGTGCAAGACAACTCAATCATCA |
| | | TTAACTGTGATGGGTTTTATCTCATCTCCCTGAAGGGTTAC |
| | | TTCTCTGAGGAGCTCAGCCTCAGCCTTTATTACCGAAAGG |
| | | GTCGGGGACCCCTCTTCTCTCTGAGCAAGGTCACATCTGT |
| | | TGACTCCATTGGAGTGGCCTATCTGGCTTTCAAGGACAAA |
| | | GTCTACTTTAATGTGACCACTCACAGTACCTCCTACAAAGA |
| | | CATCCAGGTGAATGGTGGGGAATTGATTCTCATTCATCAAA |
| | | ATCCTGGTGGCTTCTGTGCCTACTGA |
| 3 | Canine OX40 splice | ATGAGGATGTTCGTGGAGTCCCTGCGGCTCAGCGGTCCTC |
| | short variant nucleic | ACTCAGCCCTCCTGCTCCTGGGGCTTGTGCTGGGTGCCGT |
| | acid | AGCTGAGCACAACTGTTTCGGGAACACCTACCCCAAAGAC |
| | | GGCAAGTGCTGCAATGACTGCCCACCAGGTTATGGAATGG |
| | | AGAGCCGCTGCAGTAGGAGCCATGACACCAAATGTCATCA |
| | | GTGTCCATCTGGCTTCTACAATGAGGCTACAAATTACGAAC |
| | | CCTGCAAGCCCTGCACTCAGTGCAATCAGAGAAGTGGGAG |
| | | TGAACCCAAGAGGAGATGCACACCCACGCAGGACACCATC |
| | | TGCAGCTGTAAGCCAGGCACAGAGCCCCGGGACGGCTAC |
| | | AAGCGTGGAGTCGACTGTGCCCCATGCCCACCCGGACACT |
| | | TCTCCCCAGGGGATGACCAGGCCTGCAAGCCCTGGACCA |
| | | ACTGTACCtTGATGggAAGGCGTACAATGCAGCCGGCCAGC |
| | | AAGAGCTCAGACGCTGTCTGTGAGGACAGGAGCCTCCCC |
| | | GCCACACTGCCATGGGAGACCCAGAGCCCCCTGACCCGG |
| | | CCCCCTACCCCTCAGCCCACTATGGCCTGGCCCAGGACCT |
| | | CGCAGGGGCCCTTCACACCCCCTACGGAGCCCCCCAGGG |
| | | GTGGAAATAGCTTCCGGACCCCCATCCAAGAGGAGCATGC |
| | | TGACGCCAACTCCACCCTGGCCAAGATCTGA |
| 4 | Canine OX40 splice | MRMFVESLRLSGPHSALLLLGLVLGAVAEHNCFGNTYPKDGK |
| | short variant amino | CCNDCPPGYGMESRCSRSHDTKCHQCPSGFYNEATNYEPC |
| | acid | KPCTQCNQRSGSEPKRRCTPTQDTICSCKPGTEPRDGYKRG |
| | | VDCAPCPPGHFSPGDDQACKPWTNCTLMGRRTMQPASKSS |
| | | DAVCEDRSLPATLPWETQSPLTRPPTPQPTMAWPRTSQGPF |
| | | TPPTEPPRGGNSFRTPIQEEHADANSTLAKI |
| 5 | Canine OX40 splice | ATGAGGATGTTCGTGGAGTCCCTGCGGCTCAGCGGTCCTC |
| | long variant nucleic | ACTCAGCCCTCCTGCTCCTGGGGCTTGTGCTGGGTGCCGT |
| | acid | AGCTGAGCACAACTGTTTCGGGAACACCTACCCCAAAGAC |
| | | GGCAAGTGCTGCAATGACTGCCCACCAGGTTATGGAATGG |
| | | AGAGCCGCTGCAGTAGGAGCCATGACACCAAATGTCATCA |
| | | GTGTCCATCTGGCTTCTACAATGAGGCTACAAATTACGAAC |
| | | CCTGCAAGCCCTGCACTCAGTGCAATCAGAGAAGTGGGAG |
| | | TGAACCCAAGAGGAGATGCACACCCACGCAGGACACCATC |
| | | TGCAGCTGTAAGCCAGGCACAGAGCCCCGGGACGGCTAC |
| | | AAGCGTGGAGTCGACTGTGCCCCATGCCCACCCGGACACT |
| | | TCTCCCCAGGGGATGACCAGGCCTGCAAGCCCTGGACCA |
| | | ACTGTACCtTGATGggAAGGCGTACAATGCAGCCGGCCAGC |
| | | AAGAGCTCAGACGCTGTCTGTGAGGACAGGAGCCTCCCC |
| | | GCCACACTGCCATGGGAGACCCAGAGCCCCCTGACCCGG |
| | | CCCCCTACCCCTCAGCCCACTATGGCCTGGCCCAGGACCT |
| | | CGCAGGGGCCCTTCACACCCCCTACGGAGCCCCCCAGGG |
| | | GCCCCCAGCTGGCTGCTGTCCTGGGCTTGGGCCTAGGCT |
| | | TGCTGGCCCCCGTGGCAGCCGCACTGGCCTTGCTCCTGC |
| | | ACCACAGAGCCTGGCGGCTGCCCCCCGGTGGAAATAGCT |
| | | TCCGGACCCCCATCCAAGAGGAGCATGCTGACGCCAACTC |
| | | CACCCTGGCCAAGATCTGA |
| 6 | Canine OX40 splice | MRMFVESLRLSGPHSALLLLGLVLGAVAEHNCFGNTYPKDGK |
| | long variant amino acid | CCNDCPPGYGMESRCSRSHDTKCHQCPSGFYNEATNYEPC |
| | | KPCTQCNQRSGSEPKRRCTPTQDTICSCKPGTEPRDGYKRG |
| | | VDCAPCPPGHFSPGDDQACKPWTNCTLMGRRTMQPASKSS |
| | | DAVCEDRSLPATLPWETQSPLTRPPTPQPTMAWPRTSQGPF |
| | | TPPTEPPRGPQLAAVLGLGLGLLAPVAAALALLLHHRAWRLP |
| | | PGGNSFRTPIQEEHADANSTLAKI |
| 7 | OX40 Forward primer | GGCAGAGATGAGGATGTTCG |
| 8 | OX40 Reverse primer | CACTGGCTGCTCAGATCTTGG |
| 9 | OX40L Forward | GCCACAGTTTTCATCTCCCT |
| 10 | OX40L Reverse | GCTTGGCTTAGGTGCAGC |
| 11 | PMX025 Heavy chain | GAGGTGCACCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAGCTACCACATGAGTTGGGTCCGCCAGGC |
| | | TCCAGGGAAGGGACTTCAGTGGGTCGCATTCATTAACGCT |
| | | GGTGAAATTAGCACAAACTATGCAGACGCTGTGAAGGGCC |
| | | GATTCACCATCTCCAGAGACAACGCCAAGAATATATTGTAT |
| | | CTTCAGATGAACAGCCTGAGAGCCGAGGACACGGCCGTGT |
| | | ATTACTGTGCGAGTGGACATATTTGGATACGTCCCTTTGAC |
| | | TACTGGGGCCAGGGAACCCTGGTCACCGTCTCCTCAG |
| 12 | PMX025 Heavy chain | EVHLVESGGDLVKPGGSLRLSCVASGFTFSSYHMSWVRQAP |
| | variable region amino | GKGLQWVAFINAGEISTNYADAVKGRFTISRDNAKNILYLQMN |
| | acid sequence | SLRAEDTAVYYCASGHIWIRPFDYWGQGTLVTVSS |
| 13 | PMX025 light chain | TCCTATGTGCTGACACAGCTGCCATCCATGAGTGTGACCC |
| | variable region | TGAGGCAGACGGCCCGCATCACCTGTGAGGGAGACAGCA |
| | nucleotide sequence | TTGGAAGGAAAAGAGTTAACTGGTACCAGCAGAAGTTGGG |
| | | CCAGGTCCCTGTACTAATTATTTATGATGATGACAGCAGGC |
| | | CGTCAGGGATCCCTGAGCGATTCTCCGGCGCCAACTCGG |
| | | GGAACACAGCCACCCTGACCATCAGCGGGGCCCTGGCCG |
| | | AGGACGAGGCTGAATATTACTGCCAGGTGTGGGACAGCAG |
| | | TAATAATTGGGTATTCGGTGAGGGGACCCAGCTGACCGTC |
| | | CTCG |
| 14 | PMX025 light chain | SYVLTQLPSMSVTLRQTARITCEGDSIGRKRVNWYQQKLGQV |
| | variable region amino | PVLIIYDDDSRPSGIPERFSGANSGNTATLTISGALAEDEAEYY |
| | acid sequence | CQVWDSSNNWVFGEGTQLTVL |
| 15 | PMX025 heavy chain | GFTFSSY |
| | CDR1 | |
| 16 | PMX025 heavy chain | NAGEIS |
| | CDR2 | |
| 17 | PMX025 heavy chain | CASGHIWIRPFDYW |
| | CDR3 | |
| 18 | PMX025 light chain | EGDSIGRKRVN |
| | CDR1 | |
| 19 | PMX025 light chain | DDDSRPS |
| | CDR2 | |
| 20 | PMX025 light chain | CQVWDSSNNWVF |
| | CDR3 | |
| 21 | PMX026 Heavy chain | GAGGTGCACCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAGCTACCACATGAGTTGGGTCCGCCAGGC |
| | | TCCAGGGAAGGGACTTCAGTGGGTCGCATTCATTAACGCT |
| | | GGTGAAATTAGCACAAACTATGCAGACGCTGTGAAGGGCC |
| | | GATTCACCATCTCCAGAGACAACGCCAAGAATATATTGTAT |
| | | CTTCAGATGAACAGCCTGAGAGCCGAGGACACGGCCGTGT |
| | | ATTACTGTGCGAGTGGACATATTTGGATACGTCCCTTTGAC |
| | | TACTGGGGCCAGGGAACCCTGGTCACCGTCTCCTCAG |
| 22 | PMX026 Heavy chain | EVHLVESGGDLVKPGGSLRLSCVASGFTFSSYHMSWVRQAP |
| | variable region amino | GKGLQWVAFINAGEISTNYADAVKGRFTISRDNAKNILYLQMN |
| | acid sequence | SLRAEDTAVYYCASGHIWIRPFDYWGQGTLVTVSS |
| 23 | PMX026 light chain | TCCTATGTGCTGACACAGCTGCCATCCATGAGTGTGACCC |
| | variable region | TGAGGCAGACGGCCCGCATCACCTGTGAGGGAGACAGCA |
| | nucleotide sequence | TTGGAAGGAAAAGAGTTAACTGGTACCAGCAGAAGTTGGG |
| | | CCAGGTCCCTGTACTAATTATTTATGATGATGACAGCAGGC |
| | | CGTCAGGGATCCCTGAGCGATTCTCCGGCGCCAACTCGG |
| | | GGAACACAGCCACCCTGACCATCAGCGGGGCCCTGGCCG |
| | | AGGACGAGGCTGAATATTACTGCCAGGTGTGGGACAGCAG |
| | | TAATAATTGGGTATTCGGTGAGGGGACCCAGCTGACCGTC |
| | | CTCG |
| 24 | PMX026 light chain | SYVLTQLPSMSVTLRQTARITCEGDSIGRKRVNWYQQKLGQV |
| | variable region amino | PVLIIYDDDSRPSGIPERFSGANSGNTATLTISGALAEDEAEYY |
| | acid sequence | CQVWDSSNNWVFGEGTQLTVL |
| 25 | PMX026 heavy chain | GFTFSSY |
| | CDR1 | |
| 26 | PMX026 heavy chain | NAGEIS |
| | CDR2 | |
| 27 | PMX026 heavy chain | CASGHIWIRPFDYW |
| | CDR3 | |
| 28 | PMX026 light chain | EGDSIGRKRVN |
| | CDR1 | |
| 29 | PMX026 light chain | DDDSRPS |
| | CDR2 | |
| 30 | PMX026 light chain | CQVWDSSNNWVF |
| | CDR3 | |
| 31 | PMX027 Heavy chain | GAGGTGCACCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTACCTACCACATGAGTTGGGTCCGCCAGGC |
| | | TCCAGGGAAGGGACTTCAGTGGGTCGCATTCATTAACGCT |
| | | GGTGAAATTAGCACAAACTATGCAGACGCTGTGAAGGGCC |
| | | GATTCACCATCTCCAGAGACAACGCCAAGAATATATTCTAT |
| | | CTTCAGATGAACAGCCTGAGAGCCGAGGACACGGCCGTGT |
| | | ATTACTGTGCGACTGGACATATTTGGATACGTCCCTTTGAC |
| | | TACTGGGGCCAGGGAACCCTGGTCACCGTCTCCTCAG |
| 32 | PMX027 Heavy chain | EVHLVESGGDLVKPGGSLRLSCVASGFTFSTYHMSWVRQAP |
| | variable region amino | GKGLQWVAFINAGEISTNYADAVKGRFTISRDNAKNIFYLQMN |
| | acid sequence | SLRAEDTAVYYCATGHIWIRPFDYWGQGTLVTVSS |
| 33 | PMX027 light chain | TCCTATGTACTGACACAGCTGCCATCCATGAGTGTGACCCT |
| | variable region | GAGGCAGACGGCCCGCATCACCTGTGAGGGAAACAGCAT |
| | nucleotide sequence | TGGAAGGAAAAGAGTTAACTGGTACCAGCAGAAGTTGGGC |
| | | CAGGTCCCTGTACTAATTATTTATGATGATGACAGCAGGCC |
| | | GTCAGGGATCCCTGAGCGATTCTCCGGCGCCAACTCGGG |
| | | GAACACAGCCACCCTGACCATCAGCGGGGCCCTGGCCGA |
| | | GGACGAGGCTGAATATTACTGCCAGGTGTGGGACAGCAGT |
| | | AATAATTGGGTATTCGGTGAGGGGACCCAGCTGACCGTCC |
| | | TCG |
| 34 | PMX027 light chain | SYVLTQLPSMSVTLRQTARITCEGNSIGRKRVNWYQQKLGQV |
| | variable region amino | PVLIIYDDDSRPSGIPERFSGANSGNTATLTISGALAEDEAEYY |
| | acid sequence | CQVWDSSNNWVFGEGTQLTVL |
| 35 | PMX027 heavy chain | GFTFSTY |
| | CDR1 | |
| 36 | PMX027 heavy chain | NAGEIS |
| | CDR2 | |
| 37 | PMX027 heavy chain | CATGHIWIRPFDYW |
| | CDR3 | |
| 38 | PMX027 light chain | EGNSIGRKRVN |
| | CDR1 | |
| 39 | PMX027 light chain | DDDSRPS |
| | CDR2 | |
| 40 | PMX027 light chain | CQVWDSSNNWVF |
| | CDR3 | |
| 41 | PMX028 Heavy chain | GAGGTGCAGATGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTGACTACCACATGAGCTGGGTCCGCCAGGC |
| | | TCCAGGGAAGGGACTTCAGTGGGTCGCATACATTACCAGT |
| | | GGTGGAAGTAGCACAAGCTATGCAGACGCTGTGAAGGGC |
| | | CGATTCACCATCTCCAGAGACAACGCCAAGAACACGCTGT |
| | | ATCTTCAGATGAACAGCCTGAGAGCCGAGGACACGGCCGT |
| | | GTATTATTGTGCGAGTGATCTAGTTCTAGTAGTTGGCGGGG |
| | | GGGTCTACTGGGGCCAGGGAACCCTGGTCACCGTCTCCT |
| | | CAG |
| 42 | PMX028 Heavy chain | EVQMVESGGDLVKPGGSLRLSCVASGFTFSDYHMSWVRQA |
| | variable region amino | PGKGLQWVAYITSGGSSTSYADAVKGRFTISRDNAKNTLYLQ |
| | acid sequence | MNSLRAEDTAVYYCASDLVLVVGGGVYWGQGTLVTVSS |
| 43 | PMX028 light chain | TCCTATGAACTGACTCAGCCACCATCCGTGAATGTGACCCT |
| | variable region | GAGGGAGACGGCCCACATCACCTGTGGGGGAGACAGCAT |
| | nucleotide sequence | TGGAAGTAAATATGTTCAATGGATCCAGCAGAATCCAGGCC |
| | | AGGCCCCCGTGGTGATTATCTATAAAGATAGCAACAGGCC |
| | | GACAGGGATCCCTGAGCGATTCTCTGGCGCCAACTCAGGG |
| | | AACACGGCTACCCTGACCATCAGTGGGGCCCTGGCCGAA |
| | | GACGAGGCTGACTATTACTGCCAGGTGGGGGACAGTGGTA |
| | | CTAAGGCGGTATTCGGTGAAGGGACCCAGCTGACCGTCCT |
| | | CG |
| 44 | PMX028 light chain | SYELTQPPSVNVTLRETAHITCGGDSIGSKYVQWIQQNPGQA |
| | variable region amino | PVVIIYKDSNRPTGIPERFSGANSGNTATLTISGALAEDEADYY |
| | acid sequence | CQVGDSGTKAVFGEGTQLTVL |
| 45 | PMX028 heavy chain | GFTFSDY |
| | CDR1 | |
| 46 | PMX028 heavy chain | TSGGSS |
| | CDR2 | |
| 47 | PMX028 heavy chain | CASDLVLVVGGGVYW |
| | CDR3 | |
| 48 | PMX028 light chain | GGDSIGSKYVQ |
| | CDR1 | |
| 49 | PMX028 light chain | KDSNRPT |
| | CDR2 | |
| 50 | PMX028 light chain | CQVGDSGTKAVF |
| | CDR3 | |
| 51 | PMX029 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTTCCTACCACATGAACTGGGTCCGCCAGGC |
| | | TCCAGGGAAGGGGCTTCAGTGGGTCGCATACATTAACACT |
| | | GGTGGAAGTCGCACAAGCTATGCAGACGCTGTGAAGGGC |
| | | CGATTCACCATCTCCAGAGACAACGCCAAGAACACGTTGT |
| | | ATCTTCAGATGAACAGCCTGAGAGCCGAGGACACGGCCGT |
| | | GTATTACTGTGCGAATAACTACTGGGATAATTTTGACTCCT |
| | | GGGGCCAGGGAACCCTGGTCACCGTCTCCTCAG |
| 52 | PMX029 Heavy chain | EVQLVESGGDLVKPGGSLRLSCVASGFTFSSYHMNWVRQAP |
| | variable region amino | GKGLQWVAYINTGGSRTSYADAVKGRFTISRDNAKNTLYLQM |
| | acid sequence | NSLRAEDTAVYYCANNYWDNFDSWGQGTLVTVSS |
| 53 | PMX029 light chain | TCCTATGAGCTGACTCAGCCACCATCCGTGAATGTGACCC |
| | variable region | TGAGGGAGACGGCCCACATCACCTGTGGGGGAGACAGCA |
| | nucleotide sequence | TTGGAAGTAAATATGTTCAATGGATCCAGCAGAATCCAGGC |
| | | CAGGCCCCCGTGGTGATTATCTATAAAGATACCAACAGGC |
| | | CGACAGGGATCCCTGAGCGATTCTCTGGCGCCAACTCAGG |
| | | GAACACGGCTACCCTGACCATCAGTGGGGCCCTGGCCGA |
| | | AGACGAGGCTGACTATTACTGCCAGGTGGGGGACAGTGGT |
| | | ACTAAGGCTAGTGTGTTCGGCGGAGGCACCCATCTGACCG |
| | | TCCTCG |
| 54 | PMX029 light chain | SYELTQPPSVNVTLRETAHITCGGDSIGSKYVQWIQQNPGQA |
| | variable region amino | PVVIIYKDTNRPTGIPERFSGANSGNTATLTISGALAEDEADYY |
| | acid sequence | CQVGDSGTKASVFGGGTHLTVL |
| 55 | PMX029 heavy chain | GFTFSSY |
| | CDR1 | |
| 56 | PMX029 heavy chain | NTGGSR |
| | CDR2 | |
| 57 | PMX029 heavy chain | CANNYWDNFDSW |
| | CDR3 | |
| 58 | PMX029 light chain | GGDSIGSKYVQ |
| | CDR1 | |
| 59 | PMX029 light chain | KDTNRPT |
| | CDR2 | |
| 60 | PMX029 light chain | CQVGDSGTKASVF |
| | CDR3 | |
| 61 | PMX030 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAACTACCACATGAACTGGGTCCGCCAGGC |
| | | TCCAGGGAAGGGACTTCAGTGGGTCGCATACGTTGTCAGT |
| | | GGTGGAAGTACCACAAGCTATGCAGACGCTGTGAAGGGCC |
| | | GATTCACCATCTCCAGAGACAACGCCAAGAACACACTGTAT |
| | | CTTCAGATGAACAGCCTGAGAGCCGAGGACACGGCCGTGT |
| | | ATTACTGTGCGAAGGGGACTAGTAGTTGGGGGGTTCCTAT |
| | | GGACTACTGGGGCCATGGCACCTCACTCTTCGTGTCCTCA |
| | | G |
| 62 | PMX030 Heavy chain | EVQLVESGGDLVKPGGSLRLSCVASGFTFSNYHMNWVRQAP |
| | variable region amino | GKGLQWVAYVVSGGSTTSYADAVKGRFTISRDNAKNTLYLQ |
| | acid sequence | MNSLRAEDTAVYYCAKGTSSWGVPMDYWGHGTSLFVSS |
| 63 | PMX030 light chain | TCCTATGTGCTGACACAGCTGCCATCCGTGAGTGTGACCC |
| | variable region | TGAGGCAGACGGCCCGCATCACCTGTGGGGGAGACAGCA |
| | nucleotide sequence | TTAGAAGTAAAAATGTTTACTGGTACCAGCAGAAGCTGGGC |
| | | CAGGCCCCTGTACTGATTATCTATGATGATAGCAGCAGGC |
| | | CGTCAGGGATCCCTGAGCGATTCTCCGGCGCCAACTCGG |
| | | GGAACACGGCCACCCTGACCATCAGCGGGGCCCTGGCCG |
| | | AGGATGAGGCTGACTATTACTGCCAGGTGTGGGACAGCAG |
| | | TACTAGTGTGTTCGGCGGAGGCACCCATCTGACCGTCCTC |
| | | G |
| 64 | PMX030 light chain | SYVLTQLPSVSVTLRQTARITCGGDSIRSKNVYWYQQKLGQA |
| | variable region amino | PVLIIYDDSSRPSGIPERFSGANSGNTATLTISGALAEDEADYY |
| | acid sequence | CQVWDSSTSVFGGGTHLTVL |
| 65 | PMX030 heavy chain | GFTFSNY |
| | CDR1 | |
| 66 | PMX030 heavy chain | VSGGST |
| | CDR2 | |
| 67 | PMX030 heavy chain | CAKGTSSWGVPMDYW |
| | CDR3 | |
| 68 | PMX030 light chain | GGDSIRSKNVY |
| | CDR1 | |
| 69 | PMX030 light chain | DDSSRPS |
| | CDR2 | |
| 70 | PMX030 light chain | CQVWDSSTSVF |
| | CDR3 | |
| 71 | PMX031 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAACTACCACATGAACTGGGTCCGCCAGGC |
| | | TCCAGGGAAGGGACTTCAGTGGGTCGCATACGTTGTCAGT |
| | | GGTGGAACTACCACGACCTATGCAGACGCTGTGAAGGGCC |
| | | GATTCACCATCTCCAGAGACAACGCCAAGAACACACTGTAT |
| | | CTTCAGATGAACAGCCTGAGAGCCGAGGACACGGCCGTGT |
| | | ATTACTGTGCGAAGGGGACTAGTACTTGGGGGGTTCCTAT |
| | | GGACTACTGGGGCCATGGCACCTCACTCTTCGTGTCCTCA |
| | | G |
| 72 | PMX031 Heavy chain | EVQLVESGGDLVKPGGSLRLSCVASGFTFSNYHMNWVRQAP |
| | variable region amino | GKGLQWVAYVVSGGTTTTYADAVKGRFTISRDNAKNTLYLQ |
| | acid sequence | MNSLRAEDTAVYYCAKGTSTWGVPMDYWGHGTSLFVSS |
| 73 | PMX031 light chain | TCCTATGTGCTGACACAGCTGCCATCCGTGAGTGTGACCC |
| | variable region | TGAGGCAGACGGCCCGCATCACCTGTGGGGGAGACAGCA |
| | nucleotide sequence | TTAGAAGTAAAAATGTTTACTGGTACCAGAAGAAGCTGGGC |
| | | CAGGCCCCTGTACTGATTATCTATGATGATAGCAGCAGGC |
| | | CGTCAGGGATCCCTGAGCGATTCTCCGGCGCCAACTCGG |
| | | GGAACACGGCCACCCTGACCATCAGCGGGGCCCTGGCCG |
| | | AGGATGAGGCTGACTATTACTGCCAGGTGTGGGACAGCAG |
| | | TACTAGTGTGTTCGGCGGAGGCACCCATCTGACCGTCCTC |
| | | G |
| 74 | PMX031 light chain | SYVLTQLPSVSVTLRQTARITCGGDSIRSKNVYWYQKKLGQA |
| | variable region amino | PVLIIYDDSSRPSGIPERFSGANSGNTATLTISGALAEDEADYY |
| | acid sequence | CQVWDSSTSVFGGGTHLTVL |
| 75 | PMX031 heavy chain | GFTFSNY |
| | CDR1 | |
| 76 | PMX031 heavy chain | VSGGTT |
| | CDR2 | |
| 77 | PMX031 heavy chain | CAKGTSTWGVPMDYW |
| | CDR3 | |
| 78 | PMX031 light chain | GGDSIRSKNVY |
| | CDR1 | |
| 79 | PMX031 light chain | DDSSRPS |
| | CDR2 | |
| 80 | PMX031 light chain | CQVWDSSTSVF |
| | CDR3 | |
| 81 | PMX032 Heavy chain | GAGGTGCAGCTGGTGGAGTCTGGGGGAGACCTGGTGAAG |
| | variable region nucleic | CCGGGGGGGTCCCTGAGACTTTCCTGTGTGGCCTCTGGAT |
| | acid sequence | TCACCTTCAGCAGTTATCACATGAGCTGGGTCCGCCAGGC |
| | | TCCAGGGAAGGGGCTTCAGTGGGTCGCATACATTACCAGT |
| | | GGTGGAAGAACCACAAGCTATGCAGACGCTGTGAAGGGC |
| | | CGATTCATCATCTCCAGAGACAACGCCAAGAACACGTTGTA |
| | | TCTTCAGATGAACAGCCTGAGAGCCGAGGACACGGCCGTG |
| | | TATTACTGTGCGAGTGATCTGGTTCTAATATTAGGGGGGGC |
| | | TGACTACTGGGGCCAGGGAACCCTGGTCACCGTCTCCTCA |
| | | G |
| 82 | PMX032 Heavy chain | EVQLVESGGDLVKPGGSLRLSCVASGFTFSSYHMSWVRQAP |
| | variable region amino | GKGLQWVAYITSGGRTTSYADAVKGRFIISRDNAKNTLYLQM |
| | acid sequence | NSLRAEDTAVYYCASDLVLILGGADYWGQGTLVTVSS |
| 83 | PMX032 light chain | TCCTATGAGCTGACTCAGCCACCATCCGTGAATGTGACCC |
| | variable region | TGAGGGAGACGGCCCACATCACCTGTGGGGGAGACAGCA |
| | nucleotide sequence | TTGGAAGGAAATATGTTCAATGGATCCAGCAGAATCCAGG |
| | | CCAGGCCCCCGTGGTGATTATCTATAAAGATAACAACAGG |
| | | CCGACAGGGATCCCTGAGCGATTCTCTGGCGCCAACTCAG |
| | | GGAACACGGCTACCCTGACCATCAGTGGGGCCCTGGCCG |
| | | AAGACGAGGCTGACTATTACTGCCAGGTGGGGGACAGTG |
| | | GTTCTAAGGCTGTGTTCGGCGGAGGCACCCACCTGACCGT |
| | | CCTCG |
| 84 | PMX032 light chain | SYELTQPPSVNVTLRETAHITCGGDSIGRKYVQWIQQNPGQA |
| | variable region amino | PVVIIYKDNNRPTGIPERFSGANSGNTATLTISGALAEDEADYY |
| | acid sequence | CQVGDSGSKAVFGGGTHLTVL |
| 85 | PMX032 heavy chain | GFTFSSY |
| | CDR1 | |
| 86 | PMX032 heavy chain | TSGGRT |
| | CDR2 | |
| 87 | PMX032 heavy chain | CASDLVLILGGADYW |
| | CDR3 | |
| 88 | PMX032 light chain | GGDSIGRKYVQ |
| | CDR1 | |
| 89 | PMX032 light chain | KDNNRPT |
| | CDR2 | |
| 90 | PMX032 light chain | CQVGDSGSKAVF |
| | CDR3 | |
| 91 | PMX033 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAGTTATCACATGAGCTGGGTCCGCCAGGC |
| | | TCCAGGGAAGGGGCTTCAGTGGGTCGCATACATTACCAGT |
| | | GGTGGAAGAACCACAAGCTATGCAGACGCTGTGAAGGGC |
| | | CGATTCACCATCTCCAGAGACAATGCCAAGAACACGTTGTA |
| | | TCTTCAGATGAAAAGCCTGAGAGCCGAGGACACGGCCGTG |
| | | TATTACTGTGCGAGTGATCTGGTTCTAGTATTAGGGGGGG |
| | | CTGAGTACTGGGGCCAGGGAACCCTGGTCACCGTCTCCTC |
| | | AG |
| 92 | PMX033 Heavy chain | EVQLVESGGDLVKPGGSLRLSCVASGFTFSSYHMSWVRQAP |
| | variable region amino | GKGLQWVAYITSGGRTTSYADAVKGRFTISRDNAKNTLYLQM |
| | acid sequence | KSLRAEDTAVYYCASDLVLVLGGAEYWGQGTLVTVSS |
| 93 | PMX033 light chain | TCCTATGAGCTGACTCAGCCACCATCCGTGAATGTGACCC |
| | variable region | TGAGGGAGACGGCCCACATCACCTGTGGGGGAGACAGCA |
| | nucleotide sequence | TTGGAAGGAAATATGTTCAATGGATCCAGCAGAATCCAGG |
| | | CCAGGCCCCCGTCGTGATTATCTATAAAGATAGCAACAGG |
| | | CCGACAGGGATCCCTGAGCGATTCTCTGGCGCCAACTCAG |
| | | GGAACACGGCTACCCTGACCATCAGTGGGGCCCTGGCCG |
| | | AAGACGAGGCTGACTATTACTGCCAGGTGGGGGACAGTG |
| | | GTTCTAAGGCTGTGTTCGGCGGAGGCACCCACCTGACCGT |
| | | CCTCG |
| 94 | PMX033 light chain | SYELTQPPSVNVTLRETAHITCGGDSIGRKYVQWIQQNPGQA |
| | variable region amino | PVVIIYKDSNRPTGIPERFSGANSGNTATLTISGALAEDEADYY |
| | acid sequence | CQVGDSGSKAVFGGGTHLTVL |
| 95 | PMX033 heavy chain | GFTFSSY |
| | CDR1 | |
| 96 | PMX033 heavy chain | TSGGRT |
| | CDR2 | |
| 97 | PMX033 heavy chain | CASDLVLVLGGAEYW |
| | CDR3 | |
| 98 | PMX033 light chain | GGDSIGRKYVQ |
| | CDR1 | |
| 99 | PMX033 light chain | KDSNRPT |
| | CDR2 | |
| 100 | PMX033 light chain | CQVGDSGSKAVF |
| | CDR3 | |
| 101 | PMX034 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAATTATCACATGAGCTGGGTCCGCCAGGCT |
| | | CCAGGGAAGGGGCTTCAGTGGGTCGCATACATTGTCAGTG |
| | | GTGGAAGTAGCACAACCTATGCAGACGCTGTGAAGGGCCG |
| | | ATTCACCATCTCCAGAGACAACGCCAAGAACACGCTGTATC |
| | | TTCAGATGACCAGCCTACGAGCCGAGGATACGGCCGTGTA |
| | | TTACTGTGCGAGTGATCTGGTTCTACTAGTAGGGGGGGCT |
| | | GACTACTGGGGCCAGGGAACCCTGGTCACCGTCTCCTCAG |
| 102 | PMX034 Heavy chain | EVQLVESGGDLVKPGGSLRLSCVASGFTFSNYHMSWVRQAP |
| | variable region amino | GKGLQWVAYIVSGGSSTTYADAVKGRFTISRDNAKNTLYLQM |
| | acid sequence | TSLRAEDTAVYYCASDLVLLVGGADYWGQGTLVTVSS |
| 103 | PMX034 light chain | TCCTATGAGCTGACTCAGCCACCATCCGTGAATGTGACCC |
| | variable region | TGAGGGAGACGGCCCACATCACCTGTGGGGGAGACAGCA |
| | nucleotide sequence | TTGGAAGTAAATATGTTCAATGGATCCAGCAGAATCCAGGC |
| | | CAGGCCCCCGTGGTGATTATCTATAAAGATAGCAACAGGC |
| | | CGACAGGGATCCCTGAGCGATTCTCTGGCGCCAACTCAGG |
| | | GAACACGGCTACCCTGACCATCAGTGGGGCCCTGGCCGA |
| | | AGACGAGGCTGACTATTACTGCCAGGTGGGGGACAGTGGT |
| | | ACTAAGGCTGTGTTCGGCGGAGGCACCCACCTGACCGTCC |
| | | TCG |
| 104 | PMX034 light chain | SYELTQPPSVNVTLRETAHITCGGDSIGSKYVQWIQQNPGQA |
| | variable region amino | PVVIIYKDSNRPTGIPERFSGANSGNTATLTISGALAEDEADYY |
| | acid sequence | CQVGDSGTKAVFGGGTHLTVL |
| 105 | PMX034 heavy chain | GFTFSNY |
| | CDR1 | |
| 106 | PMX034 heavy chain | VSGGSS |
| | CDR2 | |
| 107 | PMX034 heavy chain | CASDLVLLVGGADYW |
| | CDR3 | |
| 108 | PMX034 light chain | GGDSIGSKYVQ |
| | CDR1 | |
| 109 | PMX034 light chain | KDSNRPT |
| | CDR2 | |
| 110 | PMX034 light chain | CQVGDSGTKAVF |
| | CDR3 | |
| 111 | PMX035 Heavy chain | GAGGTGCAGCTGGTGGAGTCTGGGGGAGACCTGGTGAAG |
| | variable region nucleic | CCTGGGGGGTCCCTGAGACTCTCCTGTGTGGCCTCTGGAT |
| | acid sequence | TCACCTTCAGTAACTTCGAAATGTACTGGGTCCGCCAGGCT |
| | | CCAGGGAAAGGGCTGGAGTGGGTCGCAAGGATTTATAAGA |
| | | GTGGAAGTACCACATACCATTCAGAACGTGTAAAGGGCCG |
| | | ATTCACCATCTCCAAAGACAGCGCCACGAACATGGCGTTT |
| | | CTGCAGATGAACAGCCTGAGAGCCGAGGACACGGCCGTA |
| | | TATTACTGTACGAGTGCTAGTTCTACTGGTTGGCGTTTTGA |
| | | CTACTGGGGCCAGGGAACCCTGGTCACCGTCTCCTCAG |
| 112 | PMX035 Heavy chain | EVQLVESGGDLVKPGGSLRLSCVASGFTFSNFEMYWVRQAP |
| | variable region amino | GKGLEWVARIYKSGSTTYHSERVKGRFTISKDSATNMAFLQM |
| | acid sequence | NSLRAEDTAVYYCTSASSTGWRFDYWGQGTLVTVSS |
| 113 | PMX035 light chain | TCCTATGTGCTGACACAGTTGCCATCCGTGAGTGTGACCC |
| | variable region | TGAGGCAGACGGCCCGCATCACCTGTGGGGGAGACAGCA |
| | nucleotide sequence | TTGGAAGAAAAAATGTTTGCTGGTTCCAGCAGAAGCTGGG |
| | | CCAGGCCCCTGTACTGATTATCTATGATGATAACAGCAGGC |
| | | CGTCAGGGATCCCTGAGCGATTCTCCGGCGCCAACTCGG |
| | | GGAACACGGCCACCCTGACCATCAGCGGGGCCCTGGCCG |
| | | AGGATGAGGCTGACTATTACTGCCAGGTGTGGGACAGCAG |
| | | TACTGGTATATTCGGCGGAGGCACCCATCTGACCGTCCTC |
| | | G |
| 114 | PMX035 light chain | SYVLTQLPSVSVTLRQTARITCGGDSIGRKNVCWFQQKLGQA |
| | variable region amino | PVLIIYDDNSRPSGIPERFSGANSGNTATLTISGALAEDEADYY |
| | acid sequence | CQVWDSSTGIFGGGTHLTVL |
| 115 | PMX035 heavy chain | GFTFSNF |
| | CDR1 | |
| 116 | PMX035 heavy chain | YKSGST |
| | CDR2 | |
| 117 | PMX035 heavy chain | CTSASSTGWRFDYW |
| | CDR3 | |
| 118 | PMX035 light chain | GGDSIGRKNVC |
| | CDR1 | |
| 119 | PMX035 light chain | DDNSRPS |
| | CDR2 | |
| 120 | PMX035 light chain | CQVWDSSTGIF |
| | CDR3 | |
| 121 | PMX036 Heavy chain | GAGGTGCAGTTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAGTTATCACATGAGCTGGGTCCGCCAGGC |
| | | TCCAGGGAAGGGGCTTCAGTGGGTCGCATACATTACCAGT |
| | | GGTGGAAGAACCACAAGCTATGCAGACGCTGTGAAGGGC |
| | | CGATTCACCATCTCCAGAGACAACGCCAAGAACACGCTGT |
| | | ATCTTCAGATGAACAGCCTGAGAGCCGAGGACACGGCCGT |
| | | GTATTACTGTGCGAGTGATCTGGTTCTAGTATTAGGGGGG |
| | | GCTGACTACTGGGGCCAGGGAACCCTGGTCACCGTCTCCT |
| | | CAG |
| 122 | PMX036 Heavy chain | EVQLVESGGDLVKPGGSLRLSCVASGFTFSSYHMSWVRQAP |
| | variable region amino | GKGLQWVAYITSGGRTTSYADAVKGRFTISRDNAKNTLYLQM |
| | acid sequence | NSLRAEDTAVYYCASDLVLVLGGADYWGQGTLVTVSS |
| 123 | PMX036 light chain | TCCTATGAGCTGACTCAGCCACCATCCGTGAATGTGACCC |
| | variable region | TGAGGGAGACGGCCCACATCACCTGTGGGGGAGACAGCA |
| | nucleotide sequence | TTGGAAGGAAATATGTTCAATGGATCCAGCAGAATCCAGG |
| | | CCAGGCCCCCGTGGTGATTATCTATAAAGATACCAACAGG |
| | | CCGACAGGGATCCCTGAGCGATTCTCTGGCGCCAACTCAG |
| | | GGAACACGGCTACCCTGACCATCAGTGGGGCCCTGGCCG |
| | | AAGACGAGGCTGACTATTACTGCCAGGTGGGGGACAGTG |
| | | GTACTAAGGCTGTGTTCGGCGGAGGCACCCACCTGACCGT |
| | | CCTCG |
| 124 | PMX036 light chain | SYELTQPPSVNVTLRETAHITCGGDSIGRKYVQWIQQNPGQA |
| | variable region amino | PVVIIYKDTNRPTGIPERFSGANSGNTATLTISGALAEDEADYY |
| | acid sequence | CQVGDSGTKAVFGGGTHLTVL |
| 125 | PMX036 heavy chain | GFTFSSY |
| | CDR1 | |
| 126 | PMX036 heavy chain | TSGGRT |
| | CDR2 | |
| 127 | PMX036 heavy chain | CASDLVLVLGGADYW |
| | CDR3 | |
| 128 | PMX036 light chain | GGDSIGRKYVQ |
| | CDR1 | |
| 129 | PMX036 light chain | KDTNRPT |
| | CDR2 | |
| 130 | PMX036 light chain | CQVGDSGTKAVF |
| | CDR3 | |
| 131 | PMX037 Heavy chain | GAGGTGCAGCTGCTGGAGTCTGGGGGAGACCTGGTGAAG |
| | variable region nucleic | CCTGGGGGGTCCCTGAGACTCTCCTGTGTGGCCTCTGGAT |
| | acid sequence | TCACCTTCAGTAACTACGAAATGTACTGGGTCCGCCAGGC |
| | | TCCAGGGAAAGGGCTGGAGTGGGTCGCAAGGTTTTATGAG |
| | | AGTGGAAGTACCACATACTCTGCAGAAGCTGTTCCGGGCC |
| | | GATTCACCATCTCCAGAGACAACGCCAAGAACATGGCCTA |
| | | TCTGCAGATGAACAGCCTGAGAGCCGAGGACACGGCCGT |
| | | ATATTACTGTGCGAGTGCTAGTTCTAGTTCTTGGCGTTTTG |
| | | ACTACTGGGGCCAGGGAACCCTGGTCACCGTCTCCTCAG |
| 132 | PMX037 Heavy chain | EVQLLESGGDLVKPGGSLRLSCVASGFTFSNYEMYWVRQAP |
| | variable region amino | GKGLEWVARFYESGSTTYSAEAVPGRFTISRDNAKNMAYLQ |
| | acid sequence | MNSLRAEDTAVYYCASASSSSWRFDYWGQGTLVTVSS |
| 133 | PMX037 light chain | TCCTATGTGCTGACACAGCTGCCATCCGTGAGTGTGACCC |
| | variable region | TGGGGCAGACGGCCCGCATCACCTGTGGGGGAGACAGCA |
| | nucleotide sequence | TTGGAAGTAAAAATGTTTACTGGTTCCAGCAGAAGCTGGGC |
| | | CAGGCCCCTGTACTGATTATCTATGATGATCGCAGCAGGC |
| | | CGTCAGGGATCCCTGAGCGATTCTCCGGCGCCAACTCGG |
| | | GGAACACGGCCACCCTGACCATCAGCGGGGCCCTGGCCG |
| | | AGGATGAGGCTGACTATTACTGCCAGGTGTGGGACAGCAG |
| | | TACTGGTGTGTTCGGCGGAGGCACCCATCTGACCGTCCTC |
| | | G |
| 134 | PMX037 light chain | SYVLTQLPSVSVTLGQTARITCGGDSIGSKNVYWFQQKLGQA |
| | variable region amino | PVLIIYDDRSRPSGIPERFSGANSGNTATLTISGALAEDEADYY |
| | acid sequence | CQVWDSSTGVFGGGTHLTVL |
| 135 | PMX037 heavy chain | GFTFSNY |
| | CDR1 | |
| 136 | PMX037 heavy chain | YESGST |
| | CDR2 | |
| 137 | PMX037 heavy chain | CASASSSSWRFDYW |
| | CDR3 | |
| 138 | PMX037 light chain | GGDSIGSKNVY |
| | CDR1 | |
| 139 | PMX037 light chain | DDRSRPS |
| | CDR2 | |
| 140 | PMX037 light chain | CQVWDSSTGVF |
| | CDR3 | |
| 141 | PMX038 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAGTTATCACATGAGCTGGGTCCGCCAGGC |
| | | TCCAGGGAAGGGGCTTCAGTGGGTCGCATACATTACCAGT |
| | | GGTGGAAGAACCACAAGCTATGCAGACGCTGTGAAGGGC |
| | | CGATTCACCATCTCCAGAGACAATGCCAAGAACACGTTGTA |
| | | TCTTCAGATGAAAAGCCTGAGAGCCGAGGACACGGCCGTG |
| | | TATTACTGTGCGAGGGATCTGGTTCTAGTATTAGGGGGGG |
| | | CTGAGTACTGGGGCCAGGGAACCCTGGTCACCGTCTCCTC |
| | | AG |
| 142 | PMX038 Heavy chain | EVQLVESGGDLVKPGGSLRLSCVASGFTFSSYHMSWVRQAP |
| | variable region amino | GKGLQWVAYITSGGRTTSYADAVKGRFTISRDNAKNTLYLQM |
| | acid sequence | KSLRAEDTAVYYCARDLVLVLGGAEYWGQGTLVTVSS |
| 143 | PMX038 light chain | TCCTATGAGCTGACTCAGCCACCATCCGTGAATGTGACCC |
| | variable region | TGAGGGAGACGGCCCACATCACCTGTGGGGGAGACAGCA |
| | nucleotide sequence | TTGGAAGGAAATATGTTCAATGGATCCAGCAGAATCCAGG |
| | | CCAGGCCCCCGTCGTGATTATCTATAAAGATAGCAACAGG |
| | | CCGACAGGGATCCCTGAGCGATTCTCTGGCGCCAACTCAG |
| | | GGAACACGGCTACCCTGACCATCAGTGGGGCCCTGGCCG |
| | | AAGACGAGGCTGACTATTACTGCCAGGTGGGGGACAGTG |
| | | GTTCTAAGGCTGTGTTCGGCGGAGGCACCCACCTGACCGT |
| | | CCTCG |
| 144 | PMX038 light chain | SYELTQPPSVNVTLRETAHITCGGDSIGRKYVQWIQQNPGQA |
| | variable region amino | PVVIIYKDSNRPTGIPERFSGANSGNTATLTISGALAEDEADYY |
| | acid sequence | CQVGDSGSKAVFGGGTHLTVL |
| 145 | PMX038 heavy chain | GFTFSSY |
| | CDR1 | |
| 146 | PMX038 heavy chain | TSGGRT |
| | CDR2 | |
| 147 | PMX038 heavy chain | CARDLVLVLGGAEYW |
| | CDR3 | |
| 148 | PMX038 light chain | GGDSIGRKYVQ |
| | CDR1 | |
| 149 | PMX038 light chain | KDSNRPT |
| | CDR2 | |
| 150 | PMX038 light chain | CQVGDSGSKAVF |
| | CDR3 | |
| 151 | PMX039 Heavy chain | GAGGTGCAGCTGGTGGAGTCTGGGGGAGACCTTGTGAAA |
| | variable region nucleic | CCTGAGGGGTCCCTGAGACTCTCCTGTGTGGTCTCTGGCT |
| | acid sequence | TCACCTTCATTACCAACGACATGAGCTGGGTCCGCCAGGC |
| | | TCCAGGGAAGGGGCTGCAGTGGGTCGCATACATTACCAAT |
| | | GATGGAAGGTACACAAGTTACACAGACGCTGTGAAGGGCC |
| | | GATTCACCATCTCCAGAGACAATGCCAAGAACACGCTGTAT |
| | | CTGCAGATGAACAGCCTGAGAACTGAGGACACAGCCGTTT |
| | | ATTACTGTGCGAAGCATAGTAGCAACTACTACAGGGGGAG |
| | | GTTTGGTATGGACTACTGGGGCCATGGCACCTCACTCTTC |
| | | GTGTCCTCAG |
| 152 | PMX039 Heavy chain | EVQLVESGGDLVKPEGSLRLSCVVSGFTFITNDMSWVRQAP |
| | variable region amino | GKGLQWVAYITNDGRYTSYTDAVKGRFTISRDNAKNTLYLQM |
| | acid sequence | NSLRTEDTAVYYCAKHSSNYYRGRFGMDYWGHGTSLFVSS |
| 153 | PMX039 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTACTGATTATCTATTATGATAGCACCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAGTGTGTTCGGCGGAGGCACCCATCTGACCGTCCTCG |
| 154 | PMX039 light chain | SYVLTQLPSKNVTLKQPAHITCGGDNIGSKSVHWYQQKLGQA |
| | variable region amino | PVLIIYYDSTRPTGIPERFSGANSGNTATLTISGALAEDEADYY |
| | acid sequence | CQVWDSSASVFGGGTHLTVL |
| 155 | PMX039 heavy chain | GFTFITN |
| | CDR1 | |
| 156 | PMX039 heavy chain | TNDGRY |
| | CDR2 | |
| 157 | PMX039 heavy chain | CAKHSSNYYRGRFGMDYW |
| | CDR3 | |
| 158 | PMX039 light chain | GGDNIGSKSVH |
| | CDR1 | |
| 159 | PMX039 light chain | YDSTRPT |
| | CDR2 | |
| 160 | PMX039 light chain | CQVWDSSASVF |
| | CDR3 | |
| 161 | PMX040 Heavy chain | GAGGTGCAGCTGGTGGAGTCTGGGGGAGACCTGGTGAAG |
| | variable region nucleic | CCTGGGGGGTCCCTGAGACTCTCCTGTGTGGCCTCTGGAT |
| | acid sequence | TCACCTTCAGTAACTTCGAAATGTACTGGGTCCGCCAGGCT |
| | | CCAGGGAAAGGGCTGGAGTGGGTCGCAAGGATTTATGAG |
| | | AGTGGAACTACCACATACCATGCAGAAGGTGTAAAGGGCC |
| | | GATTCACCATTTCCAAAGACAACGTCAAGAACATGGCGTAT |
| | | CTGCAGATGAACAGCCTGAGAGCCGAGGACACGGCCGTA |
| | | TATTACTGTACGAGTGCTAGTTCTAGTAGTTGGCGTTTTGA |
| | | CTACTGGGGCCAGGGAACCCTGGTCACCGTCTCCTCAG |
| 162 | PMX040 Heavy chain | EVQLVESGGDLVKPGGSLRLSCVASGFTFSNFEMYWVRQAP |
| | variable region amino | GKGLEWVARIYESGTTTYHAEGVKGRFTISKDNVKNMAYLQM |
| | acid sequence | NSLRAEDTAVYYCTSASSSSWRFDYWGQGTLVTVSS |
| 163 | PMX040 light chain | TCCTATGTGCTGACACAGCTGCCATCCGTGAGTGTGACCC |
| | variable region | TGAGGCAGACGGCCCGCATCACCTGTGGGGGAGACAGCA |
| | nucleotide sequence | TTGGAAGAAAAAATGTTTGCTGGTTCCAGCAGAAGCTGGG |
| | | CCAGGCCCCTATACTGATTATCTATGATGATAGCAGCAGGC |
| | | CGTCAGGGATCCCTGAGCGATTCTCCGGCGCCAACTCGG |
| | | GGAACACGGCCACCCTGACCATCAGCGGGGCCCTGGCCG |
| | | AGGATGAGGCTGACTATTACTGCCAGGTGTGGGACAGCAG |
| | | TACTGGTATATTCGGCGGAGGCACCCATCTGACCGTCCTC |
| | | G |
| 164 | PMX040 light chain | SYVLTQLPSVSVTLRQTARITCGGDSIGRKNVCWFQQKLGQA |
| | variable region amino | PILIIYDDSSRPSGIPERFSGANSGNTATLTISGALAEDEADYY |
| | acid sequence | CQVWDSSTGIFGGGTHLTVL |
| 165 | PMX040 heavy chain | GFTFSNF |
| | CDR1 | |
| 166 | PMX040 heavy chain | YESGTT |
| | CDR2 | |
| 167 | PMX040 heavy chain | CTSASSSSWRFDYW |
| | CDR3 | |
| 168 | PMX040 light chain | GGDSIGRKNVC |
| | CDR1 | |
| 169 | PMX040 light chain | DDSSRPS |
| | CDR2 | |
| 170 | PMX040 light chain | CQVWDSSTGIF |
| | CDR3 | |
| 171 | PMX041 Heavy chain | GAGGTGCATCTGGTGGAGTCTGGGGGAGACCTTGTGAAAC |
| | variable region nucleic | CTGAGGGGTCCCTGAGACTCTCCTGTGTGGTCTCTGGCTT |
| | acid sequence | CACCTTCACTACTAACGACATGAGCTGGGTCCGCCAGGCT |
| | | CCAGGGAAGGGGCTGCAGTGGGTCGCATACATTACCAGT |
| | | GATGGAAGGATCACAACTTACACAGACGCTGTGAAGGGCC |
| | | GATTCACCATCTCCAGAGACAATGCCAAGAACACGCTGTAT |
| | | CTGCAGATGAACAGCCTGAGAACTGAGGACACAGCCGTGT |
| | | ATTATTGTGCGAAACATAGTAGTAACTGGTCCAGGGGGAG |
| | | GTTTGGTTTGGACTACTGGGGCCATGGCACCTCACTCTTC |
| | | GTGTCCTCAG |
| 172 | PMX041 Heavy chain | EVHLVESGGDLVKPEGSLRLSCVVSGFTFTTNDMSWVRQAP |
| | variable region amino | GKGLQWVAYITSDGRITTYTDAVKGRFTISRDNAKNTLYLQMN |
| | acid sequence | SLRTEDTAVYYCAKHSSNWSRGRFGLDYWGHGTSLFVSS |
| 173 | PMX041 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTACTGATTATCTATTATGATAGCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAACAGTG |
| | | CTAGTGTGTTCGGCGGAGGCACCCATCTGACCGTCCTCG |
| 174 | PMX041 light chain | SYVLTQLPSKNVTLKQPAHITCGGDNIGSKSVHWYQQKLGQA |
| | variable region amino | PVLIIYYDSSRPTGIPERFSGANSGNTATLTISGALAEDEADYY |
| | acid sequence | CQVWDNSASVFGGGTHLTVL |
| 175 | PMX041 heavy chain | GFTFTTN |
| | CDR1 | |
| 176 | PMX041 heavy chain | TSDGRI |
| | CDR2 | |
| 177 | PMX041 heavy chain | CAKHSSNWSRGRFGLDYW |
| | CDR3 | |
| 178 | PMX041 light chain | GGDNIGSKSVH |
| | CDR1 | |
| 179 | PMX041 light chain | YDSSRPT |
| | CDR2 | |
| 180 | PMX041 light chain | CQVWDNSASVF |
| | CDR3 | |
| 181 | PMX042 Heavy chain | GAAGTGCAGCTGGTGGAGTCTGGGGGAGACCTCGTGAAG |
| | variable region nucleic | CCTGGGGGGTCCCTGAGACTTTCCTGTGTGGCCTCTGGAT |
| | acid sequence | TCACCTTCAGTAGTTATCACATGAGCTGGGTCCGCCAGGC |
| | | TCCAGGGAAGGGGCTTCAGTGGGTCGCATACATTACCAGT |
| | | GGTGGAAGTACCACAAGCTATGCAGACGCTGTGAAGGGCC |
| | | GATTCACCATCTCCAGAGACAACGCCAAGAACACGCTGTA |
| | | TCTTCAGATGGACAGCCTGAGAGCCGACGACACGGCCGT |
| | | GTATTACTGTGCGAGTGATCTGGTTCTGCTATTAGGGGGG |
| | | GCTGACTACTGGGGCCAGGGAACCCTGGTCACCGTCTCCT |
| | | CAG |
| 182 | PMX042 Heavy chain | EVQLVESGGDLVKPGGSLRLSCVASGFTFSSYHMSWVRQAP |
| | variable region amino | GKGLQWVAYITSGGSTTSYADAVKGRFTISRDNAKNTLYLQM |
| | acid sequence | DSLRADDTAVYYCASDLVLLLGGADYWGQGTLVTVSS |
| 183 | PMX042 light chain | TCCTATGAGCTGACTCAGCCACCATCCGTGAATGTGACCC |
| | variable region | TGAGGGAGACGGCCCACATCACCTGTGGGGGAGACAGCA |
| | nucleotide sequence | TTGGAAGGAAATATGTTCAATGGATCCAGCAGCATCCAGG |
| | | CCAGGCCCCCGTGGTGATTATCTATAAAGATTACAACAGG |
| | | CCGACAGGGATCCCTGAGCGATTCTCTGGCGCCAACTCAG |
| | | GGAACACGGCTACCCTGACCATCAGTGGGGCCCTGGCCG |
| | | AAGACGAGGCTGACTATTACTGCCAGGTGGGGGACAGTG |
| | | GTACTAAGGCTGTGTTCGGCGGAGGCACCCACCTGACCGT |
| | | CCTCG |
| 184 | PMX042 light chain | SYELTQPPSVNVTLRETAHITCGGDSIGRKYVQWIQQHPGQA |
| | variable region amino | PVVIIYKDYNRPTGIPERFSGANSGNTATLTISGALAEDEADYY |
| | acid sequence | CQVGDSGTKAVFGGGTHLTVL |
| 185 | PMX042 heavy chain | GFTFSSY |
| | CDR1 | |
| 186 | PMX042 heavy chain | TSGGST |
| | CDR2 | |
| 187 | PMX042 heavy chain | CASDLVLLLGGADYW |
| | CDR3 | |
| 188 | PMX042 light chain | GGDSIGRKYVQ |
| | CDR1 | |
| 189 | PMX042 light chain | KDYNRPT |
| | CDR2 | |
| 190 | PMX042 light chain | CQVGDSGTKAVF |
| | CDR3 | |
| 191 | PMX043 Heavy chain | GAGGTGCAGCTGGTGGAGTCTGGGGGAGACCTTGTGAAA |
| | variable region nucleic | CCTGAGGGGTCCCTGAGACTCTCCTGTGTGGTCTCTGGCT |
| | acid sequence | TCACCTTCAGTAGCTACGACATGAGCTGGGTCCGCCAGGC |
| | | TCCAGGGAAGGGGCTGCAGTGGGTCGCATACATCACCAAT |
| | | GACGGAAGGTACACAAGTTACACAGACGCTGTGAAGGGCC |
| | | GATTCACCATCTCCAGAGACAATGCCAAGAACACGCTGTAT |
| | | CTGCAGATGAACAGCCTGAGAACTGAGGACACAGCCATCT |
| | | ATTACTGTGCGAAGCATGGTAGCAGCTGGTCCAGGGGGAG |
| | | ATTTGCTATGGACTACTGGGGCCAAGGCACCTCACTCTTC |
| | | GTGTCCTCAG |
| 192 | PMX043 Heavy chain | EVQLVESGGDLVKPEGSLRLSCVVSGFTFSSYDMSWVRQAP |
| | variable region amino | GKGLQWVAYITNDGRYTSYTDAVKGRFTISRDNAKNTLYLQM |
| | acid sequence | NSLRTEDTAIYYCAKHGSSWSRGRFAMDYWGQGTSLFVSS |
| 193 | PMX043 chain | TCCTATGTGCTGACACAGCTGCCATCCAAAAATGTGACCCT |
| | variable region | GAACCAGCCGGCCCACATCACCTGTGGGGGAGACAGCAT |
| | nucleotide sequence | TGGAACTAAAAGTGTTCACTGGTACCAGCAGAAGCTGGGC |
| | | CAGGCCCCTGTCCTGATTATCTTTTATGATAGCAGCAGGCC |
| | | GACAGGGATCCCTGAGCGATTCTCCGGCGCCAACTCGGG |
| | | GAACACGGCCACCCTGACCATCAGCGGGGCCCTGGCCGA |
| | | GGACGAGGCTGACTATTACTGCCAGGTGTGGGACAGCAAT |
| | | GCTAGTGTGTTCGGCGGAGGCACCCATCTGACCGTCCTCG |
| 194 | PMX043 light chain | SYVLTQLPSKNVTLNQPAHITCGGDSIGTKSVHWYQQKLGQA |
| | variable region amino | PVLIIFYDSSRPTGIPERFSGANSGNTATLTISGALAEDEADYY |
| | acid sequence | CQVWDSNASVFGGGTHLTVL |
| 195 | PMX043 heavy chain | GFTFSSY |
| | CDR1 | |
| 196 | PMX043 heavy chain | TNDGRY |
| | CDR2 | |
| 197 | PMX043 heavy chain | CAKHGSSWSRGRFAMDYW |
| | CDR3 | |
| 198 | PMX043 light chain | GGDSIGTKSVH |
| | CDR1 | |
| 199 | PMX043 light chain | YDSSRPT |
| | CDR2 | |
| 200 | PMX043 light chain | CQVWDSNASVF |
| | CDR3 | |
| 201 | PMX044 Heavy chain | GAGGTGCAGCTGGTGGAGTCTGGGGGAGACCCTGTGAAA |
| | variable region nucleic | CCTGAGGGGTCCCAGAGACTCTCCTGTGTGGTCTCTGGCT |
| | acid sequence | TCACCTTCAGTAGCTACGACATGAGCTGGGTCCGCCAGGC |
| | | TCCAGGGAAGGGGCTGCAGTGGGTCGCATACATTACCAGT |
| | | GATGGAAGGAGCACAAGTTACACAGACGCTGTGAAGGGCC |
| | | GATTCACCATCTCCAGAGACAATGCCAGGAACACGCTGTTT |
| | | CTGCAGATGAACAGCCTGAGAACTGAGGACACAGCCGTGT |
| | | ATTACTGTGCGAGGCATAGTAGCAGCTGGTCCAGGGGGAG |
| | | GTTTGGTATGGACTACTGGGGCCATGGCACCTCGCTCTTC |
| | | GTGTCCTCAG |
| 202 | PMX044 Heavy chain | EVQLVESGGDPVKPEGSQRLSCVVSGFTFSSYDMSWVRQA |
| | variable region amino | PGKGLQWVAYITSDGRSTSYTDAVKGRFTISRDNARNTLFLQ |
| | acid sequence | MNSLRTEDTAVYYCARHSSSWSRGRFGMDYWGHGTSLFVS |
| | | S |
| 203 | PMX044 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTACTGATTATCTATTATGATAGCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTGGTGTGTTCGGCGGAGGCACCCATCTGACCGTCCTCG |
| 204 | PMX044 light chain | SYVLTQLPSKNVTLKQPAHITCGGDNIGSKSVHWYQQKLGQA |
| | variable region amino | PVLIIYYDSSRPTGIPERFSGANSGNTATLTISGALAEDEADYY |
| | acid sequence | CQVWDSSAGVFGGGTHLTVL |
| 205 | PMX044 heavy chain | GFTFSSY |
| | CDR1 | |
| 206 | PMX044 heavy chain | TSDGRS |
| | CDR2 | |
| 207 | PMX044 heavy chain | CARHSSSWSRGRFGMDYW |
| | CDR3 | |
| 208 | PMX044 light chain | GGDNIGSKSVH |
| | CDR1 | |
| 209 | PMX044 light chain | YDSSRPT |
| | CDR2 | |
| 210 | PMX044 light chain | CQVWDSSAGVF |
| | CDR3 | |
| 211 | PMX045 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAGTTATCACATGACCTGGGTCCGCCAGGC |
| | | TCCAGGGAAGGGGCTTCAGTGGGTCGCATACATTACCAGT |
| | | GGTGGAATTACCACAAGCTATGCAGACGCTGTGAAGGGCC |
| | | GATTCACCATCTCCAGAGACAACGCCAAGAACACGCTGTA |
| | | TCTTCAGATGAGCAGCCTGAGAGCCGAGGACACGGCCATG |
| | | TATTACTGTGCGAGGGATCTGGTTCTACTATTAGGGGGGG |
| | | CTGACTACTGGGGCCAGGGAACCCTGGTCACCGTCTCCTC |
| | | AG |
| 212 | PMX045 Heavy chain | EVQLVESGGDLVKPGGSLRLSCVASGFTFSSYHMTWVRQAP |
| | variable region amino | GKGLQWVAYITSGGITTSYADAVKGRFTISRDNAKNTLYLQMS |
| | acid sequence | SLRAEDTAMYYCARDLVLLLGGADYWGQGTLVTVSS |
| 213 | PMX045 light chain | TCCTATGAGCTGACTCAGCCACCATCCGTGAATGTGACCC |
| | variable region | TGAGGGAGACGGCCCACATCACCTGTGGGGGAGACAGCA |
| | nucleotide sequence | TTGGAAGGAAATATGTTCAATGGATCCAGCAGAATCCAGG |
| | | CCAGGCCCCCGTGGTGATTATCTATAAAGATAGCAACAGG |
| | | CCGACAGGGATCCCTGAGCGATTCTCTGGCGCCAACTCAG |
| | | GGAACACGGCTACCCTGACCATCAGTGGGGCCCTGGCCG |
| | | AAGACGAGGCTGACTATTACTGCCAGGTGGGGGACAGTG |
| | | GTACTAAGGCTGTGTTCGGCGGAGGCACCCACCTGACCGT |
| | | CCTCG |
| 214 | PMX045 light chain | SYELTQPPSVNVTLRETAHITCGGDSIGRKYVQWIQQNPGQA |
| | variable region amino | PVVIIYKDSNRPTGIPERFSGANSGNTATLTISGALAEDEADYY |
| | acid sequence | CQVGDSGTKAVFGGGTHLTVL |
| 215 | PMX045 heavy chain | GFTFSSY |
| | CDR1 | |
| 216 | PMX045 heavy chain | TSGGIT |
| | CDR2 | |
| 217 | PMX045 heavy chain | CARDLVLLLGGADYW |
| | CDR3 | |
| 218 | PMX045 light chain | GGDSIGRKYVQ |
| | CDR1 | |
| 219 | PMX045 light chain | KDSNRPT |
| | CDR2 | |
| 220 | PMX045 light chain | CQVGDSGTKAVF |
| | CDR3 | |
| 221 | PMX046 Heavy chain | GAGGTGCAGCTGGTGGAGTCTGGGGGAGACCTTGTGAAA |
| | variable region nucleic | CCTGAGGGGTCCCTGAGACTCTCCTGTGTGGTCGCTGGCT |
| | acid sequence | TCACCTTCAATAGCTACGACATGAGCTGGGTCCGCCAGGC |
| | | TCCAGGGAAGGGGCTGCAGTGGGTCGCATACATTACCAGT |
| | | GATGGAAGGTTCACAAGTTACACAGACGCTGTGAAGGGCC |
| | | GATTCACCATCTCCAGAGACAATGCCAAGAACACGCTCTAT |
| | | CTGCAGATGAGCAGCCTGAGAACTGAGGACACAGCCGTGT |
| | | ATTACTGTGCGAGACATAGTAGCAGCTGGTACAGGGGGAG |
| | | GTTTGGTATGGACTTCTGGGGCCATGGCACCTCACTCTTC |
| | | GTGTCCTCAG |
| 222 | PMX046 Heavy chain | EVQLVESGGDLVKPEGSLRLSCVVAGFTFNSYDMSWVRQAP |
| | variable region amino | GKGLQWVAYITSDGRFTSYTDAVKGRFTISRDNAKNTLYLQM |
| | acid sequence | SSLRTEDTAVYYCARHSSSWYRGRFGMDFWGHGTSLFVSS |
| 223 | PMX046 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTATACTGATTATCTATTATGATAGCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAGTGTGTTCGGCGGAGGCACCCATCTGACCGTCCTCG |
| 224 | PMX046 light chain | SYVLTQLPSKNVTLKQPAHITCGGDNIGSKSVHWYQQKLGQA |
| | variable region amino | PILIIYYDSSRPTGIPERFSGANSGNTATLTISGALAEDEADYYC |
| | acid sequence | QVWDSSASVFGGGTHLTVL |
| 225 | PMX046 heavy chain | GFTFNSY |
| | CDR1 | |
| 226 | PMX046 heavy chain | TSDGRF |
| | CDR2 | |
| 227 | PMX046 heavy chain | CARHSSSWYRGRFGMDFW |
| | CDR3 | |
| 228 | PMX046 light chain | GGDNIGSKSVH |
| | CDR1 | |
| 229 | PMX046 light chain | YDSSRPT |
| | CDR2 | |
| 230 | PMX046 light chain | CQVWDSSASVF |
| | CDR3 | |
| 231 | PMX047 Heavy chain | GAGGTGCAGCTGGTGGAGTCTGGGGGAGACCTGGTGAAG |
| | variable region nucleic | CCTGGGGGGTCCCTGAGACTGTCCTGTGTGGCCTCTGGAT |
| | acid sequence | TCATCTTCAGTAGCTACAGCATGAGCTGGGTCCGCCAGGC |
| | | TCCTGAGAAGGGGCTGCAGTTGGTCGCAGGTATTAACAGC |
| | | GCTGGAAGTAGCACGTACTACACAGACGCTGTGAAGGGCC |
| | | GATTCACCATCTCCAGAGACAACGCCAAGAACACAGTGTAT |
| | | CTGCAGATGAACAGCCTGAGAGCCGAGGACACGGCCATTT |
| | | ATTACTGTGCAAAGCGTTGGGTTTTTGACTTCTGGGGCCAG |
| | | GGAACCCTGGTCACCGTCTCCTCAG |
| 232 | PMX047 Heavy chain | EVQLVESGGDLVKPGGSLRLSCVASGFIFSSYSMSWVRQAP |
| | variable region amino | EKGLQLVAGINSAGSSTYYTDAVKGRFTISRDNAKNTVYLQM |
| | acid sequence | NSLRAEDTAIYYCAKRWVFDFWGQGTLVTVSS |
| 233 | PMX047 light chain | CATATTGTCATGACACAGACCCCACTGTCCCTGTCCGTCAG |
| | variable region | CCCTGGAGAGCCGGCCTCCATCTCCTGCAAGGCCAGTCA |
| | nucleotide sequence | GAGCCTCCTGCTCAGTTATGGAAACACCTATTTGTATTGGT |
| | | TCCGACAGAAGCCAGGCCAGTCTCCACAGCGTTTGATCTA |
| | | TAAGGTCTCCAACAGAGACCCTGGGGTCCCAGACAGGTTC |
| | | AGTGGCAGCGGGTCAGGGACAGATTTCACCCTGAGAATCA |
| | | GCAGAGTGGAGGCTGATGATGCTGGAGTTTATTACTGCGG |
| | | GCAAGGTATACAAGATCCGTTCACTTTTGGCCAAGGGACC |
| | | AAACTGGAGATCAAAC |
| 234 | PMX047 light chain | HIVMTQTPLSLSVSPGEPASISCKASQSLLLSYGNTYLYWFRQ |
| | variable region amino | KPGQSPQRLIYKVSNRDPGVPDRFSGSGSGTDFTLRISRVEA |
| | acid sequence | DDAGVYYCGQGIQDPFTFGQGTKLEIK |
| 235 | PMX047 heavy chain | GFIFSSY |
| | CDR1 | |
| 236 | PMX047 heavy chain | NSAGSS |
| | CDR2 | |
| 237 | PMX047 heavy chain | CAKRWVFDFW |
| | CDR3 | |
| 238 | PMX047 light chain | KASQSLLLSYGNTYLY |
| | CDR1 | |
| 239 | PMX047 light chain | KVSNRDP |
| | CDR2 | |
| 240 | PMX047 light chain | CGQGIQDPFTF |
| | CDR3 | |
| 24 | PMX048 Heavy chain | GAGGTGCAGCTGGTGGAGTCTGGGGGAGACCTGGTGAAG |
| | variable region nucleic | CCTGGGGGGTCCCTGAGACTCTCCTGTGTGGCCTCTGGAT |
| | acid sequence | TCACCTTCAATAACTACGAAATGTACTGGGTCCGCCAGGCT |
| | | CCAGGGAAAGGGCTGGAGTGGGTCGCAAGGATTTATGAG |
| | | AGTGGAACAACCACATATTCTGCAGAAGCTGATACGGGCC |
| | | GATTCACCATCTCCAGAGACAACGCCAAGAACATGGTTTTT |
| | | CTGCAGATGAACAGCCTGAGAGCCGAGGACACGGCCGTC |
| | | TATTACTGTGCGAGTGCTAGTTCTAGTACTTGGCGTTTTGA |
| | | CTACTGGGGCCAGGGAACCCTGGTCACCGTCTCCTCAG |
| 242 | PMX048 Heavy chain | EVQLVESGGDLVKPGGSLRLSCVASGFTFNNYEMYWVRQAP |
| | variable region amino | GKGLEWVARIYESGTTTYSAEADTGRFTISRDNAKNMVFLQM |
| | acid sequence | NSLRAEDTAVYYCASASSSTWRFDYWGQGTLVTVSS |
| 243 | PMX048 light chain | TCCTATGTGCTGACACAATTGCCTTCCGTGAGTGTGACCCT |
| | variable region | GGGGCAGACGGCCCGCATCACCTGTGGGGGAGACAGCAT |
| | nucleotide sequence | TGGAAGAAAAAATGTTTACTGGTTCCAGCAGAAGCTGGGC |
| | | CAGGCCCCTGTACTGATTATCTATGATGATCGCAGCAGGC |
| | | CGTCAGGGATCCCTGAGCGATTCTCCGGCGCCAACTCGG |
| | | GGAACACGGCCACCCTGACCATCAGCGGGGCCCTGGCCG |
| | | AGGATGAGGCTGACTATTACTGCCAGGTGTGGGACAGCAG |
| | | TACTGGTGTGTTCGGCGGAGGCACCCATCTGACCGTCCTC |
| | | G |
| 244 | PMX048 light chain | SYVLTQLPSVSVTLGQTARITCGGDSIGRKNVYWFQQKLGQA |
| | variable region amino | PVLIIYDDRSRPSGIPERFSGANSGNTATLTISGALAEDEADYY |
| | acid sequence | CQVWDSSTGVFGGGTHLTVL |
| 245 | PMX048 heavy chain | GFTFNNY |
| | CDR1 | |
| 246 | PMX048 heavy chain | YESGTT |
| | CDR2 | |
| 247 | PMX048 heavy chain | CASASSSTWRFDYW |
| | CDR3 | |
| 248 | PMX048 light chain | GGDSIGRKNVY |
| | CDR1 | |
| 249 | PMX048 light chain | DDRSRPS |
| | CDR2 | |
| 250 | PMX048 light chain | CQVWDSSTGVF |
| | CDR3 | |
| 251 | PMX049 Heavy chain | GAGGTGCAGCTGGTGGAGTCTGGGGGAGACCTGGTGAAG |
| | variable region nucleic | CCTGGGGGGTCCCTGAGACTCTCCTGTGTGGCCTCTGGAT |
| | acid sequence | TCACCTTCAATAACTACGAAATGTACTGGGTCCGCCAGGCT |
| | | CCAGGGAAAGGGCTGGAGTGGGTCGCAAGGATTTATGAG |
| | | AGTGGAAGTACCACTTTTTCTGCAGAAGCTGTTACGGGCC |
| | | GATTCACCATCTCCAGAGACAACGCCAAGAACATGGTGTAT |
| | | CTGCAGATGAACAGCCTGAGAGCCGAGGACACGGCCGTG |
| | | TATTACTGTGCGAGTGCTAGTTCTAGTTCTTGGCGTTTTGA |
| | | CTACTGGGGCCAGGGAACCCTGGTCACCGTCTCCTCAG |
| 252 | PMX049 Heavy chain | EVQLVESGGDLVKPGGSLRLSCVASGFTFNNYEMYWVRQAP |
| | variable region amino | GKGLEWVARIYESGSTTFSAEAVTGRFTISRDNAKNMVYLQM |
| | acid sequence | NSLRAEDTAVYYCASASSSSWRFDYWGQGTLVTVSS |
| 253 | PMX049 light chain | TCCTATGTGCTGACACAGCTGCCATCCGTGAGTGTGACCC |
| | variable region | TGGGGCAGACGGCCCGCATCACCTGTGGGGGAGACAGCA |
| | nucleotide sequence | TTGGGCGAAAAAATGTTTGCTGGTTCCAGCAGAGGCTGGG |
| | | CCAGGCCCCTGAACTGATTATCTATGATGATCGCAGCAGG |
| | | CCGTCAGGGATCCCTGAGCGATTCTCCGGCGCCAACTCG |
| | | GGGAACACGGCCACCCTGACCATCAGCGGGGCCCTGGCC |
| | | GAGGATGAGGCTGACTATTACTGCCAGGTGTGGGACAGCA |
| | | GTACTGGTGTGTTCGGCGGAGGCACCCATCTGACCGTCCT |
| | | CG |
| 254 | PMX049 light chain | SYVLTQLPSVSVTLGQTARITCGGDSIGRKNVCWFQQRLGQA |
| | variable region amino | PELIIYDDRSRPSGIPERFSGANSGNTATLTISGALAEDEADYY |
| | acid sequence | CQVWDSSTGVFGGGTHLTVL |
| 255 | PMX049 heavy chain | GFTFNNY |
| | CDR1 | |
| 256 | PMX049 heavy chain | YESGST |
| | CDR2 | |
| 257 | PMX049 heavy chain | CASASSSSWRFDYW |
| | CDR3 | |
| 258 | PMX049 light chain | GGDSIGRKNVC |
| | CDR1 | |
| 259 | PMX049 light chain | DDRSRPS |
| | CDR2 | |
| 260 | PMX049 light chain | CQVWDSSTGVF |
| | CDR3 | |
| 261 | PMX050 Heavy chain | GAGGTGCAGCTGGTGGAGTCTGGGGGAGACCTTGTGAAA |
| | variable region nucleic | CCTGAGGGGTCCCTGAGACTCTCCTGTGTGGTCTCTGGCT |
| | acid sequence | TCACCTTCACTACTTACGACATGAGCTGGGTCCGCCAGGC |
| | | TCCAGGGAAGGGGCTGCAGTGGGTCGCATACATTACCAAT |
| | | GATGGAAGGATCACAACTTACACAGACGCTGTGAAGGGCC |
| | | GATTCACCATCTCCAGAGACAATGCCAAGAACACGCTGTAT |
| | | CTGCAGATGAACAGCCTGAGAACTGTTGACACAGGCGTGT |
| | | ATTATTGTGCGAGGCATAGTAGCAGCTGGTCCAGGGGGAG |
| | | GTTTGGTTTGGACTACTGGGGCCATGGTACTTCAGTCTTCG |
| | | TGTCCTCAG |
| 262 | PMX050 Heavy chain | EVQLVESGGDLVKPEGSLRLSCVVSGFTFTTYDMSWVRQAP |
| | variable region amino | GKGLQWVAYITNDGRITTYTDAVKGRFTISRDNAKNTLYLQM |
| | acid sequence | NSLRTVDTGVYYCARHSSSWSRGRFGLDYWGHGTSVFVSS |
| 263 | PMX050 light chain | TCCTATGTGCTGACACAGTTGCCATCCAAAAATGTGACCCT |
| | variable region | GAAGCAGCCGGCCCACATCACCTGTGGGGGAGACAACATT |
| | nucleotide sequence | GGAAGGAAAGGTGTTCACTGGTACCAGCAGAAGCTGGGC |
| | | CAGGCCCCTGTATTGATTATCTATTATGATAGCAGCAGGCC |
| | | GACAGGGATCCCTGAGCGATTCTCCGGCGCCAACTCGGG |
| | | GAACACGGCCACCCTGACCATCAGCGGGGCCCTGGCCGA |
| | | GGACGAGGCTGACTATTACTGCCAGGTGTGGGACAACAGT |
| | | GCTAGTGTGTTCGGCGGAGGCACCCATCTGACCGTCCTCG |
| 264 | PMX050 light chain | SYVLTQLPSKNVTLKQPAHITCGGDNIGRKGVHWYQQKLGQA |
| | variable region amino | PVLIIYYDSSRPTGIPERFSGANSGNTATLTISGALAEDEADYY |
| | acid sequence | CQVWDNSASVFGGGTHLTVL |
| 265 | PMX050 heavy chain | GFTFTTY |
| | CDR1 | |
| 266 | PMX050 heavy chain | TNDGRI |
| | CDR2 | |
| 267 | PMX050 heavy chain | CARHSSSWSRGRFGLDYW |
| | CDR3 | |
| 268 | PMX050 light chain | GGDNIGRKGVH |
| | CDR1 | |
| 269 | PMX050 light chain | YDSSRPT |
| | CDR2 | |
| 270 | PMX050 light chain | CQVWDNSASVF |
| | CDR3 | |
| 271 | PMX051 Heavy chain | GAGGTACAGTTGGTGGAGTCTGGGGGAGACCTGGTGAAG |
| | variable region nucleic | CCGGGGGGGTCCCTGAGACTTTCCTGTGTGGCCTCTGGAT |
| | acid sequence | TCTCCTTCAATACCTACCACATGAACTGGGTCCGCCAGGCT |
| | | CCAGGGAAGGGGCTTCACTGGGTCGCATTCATTAACAGCG |
| | | ATGGGGGTGGCACAAGTTATGCAGACACTGTGAAGGGCC |
| | | GATTCACCATCTCCAGAGACTACGCCAAGAATACACTCTAT |
| | | CTTCAGATGAATAGTCTGAGAGCCGAAGACACGGCCGTTT |
| | | ATTATTGTGTGAGTGGATCTATATGGACTCATTTTGACTACT |
| | | GGGGCCAGGGAACCCTGGTCACCGTCTCCTCAG |
| 272 | PMX051 Heavy chain | EVQLVESGGDLVKPGGSLRLSCVASGFSFNTYHMNWVRQAP |
| | variable region amino | GKGLHWVAFINSDGGGTSYADTVKGRFTISRDYAKNTLYLQM |
| | acid sequence | NSLRAEDTAVYYCVSGSIWTHFDYWGQGTLVTVSS |
| 273 | PMX051 light chain | TCCTATGTGCTGACACAGCTGCCATCCGTGAGTATGACCC |
| | variable region | TGAGGCAGACGGCCCACATCACCTGTGGGGGAGACATCAT |
| | nucleotide sequence | TGGAAGTAAAAGTGTTTATTGGTACCAGCAGAAGCTGGGC |
| | | CAGGCCCCTGTACTGATTATTTATAGAGATACCAACAGGCC |
| | | GACAGGGATCCCTGAGCGATTCTCTGGCGCCAGCTCGGG |
| | | GAACACGGCCACCCTGACCATCAGCGGGGCCCTGGCCGA |
| | | GGACGAGGCTGACTATTACTGCCAGGTTTGGGACAGCAGT |
| | | ACTCAGGCTGTGTTCGGCGGAGGCACCCATCTGACCGTCC |
| | | TCG |
| 274 | PMX051 light chain | SYVLTQLPSVSMTLRQTAHITCGGDIIGSKSVYWYQQKLGQA |
| | variable region amino | PVLIIYRDTNRPTGIPERFSGASSGNTATLTISGALAEDEADYY |
| | acid sequence | CQVWDSSTQAVFGGGTHLTVL |
| 275 | PMX051 heavy chain | GFSFNTY |
| | CDR1 | |
| 276 | PMX051 heavy chain | NSDGGG |
| | CDR2 | |
| 277 | PMX051 heavy chain | CVSGSIWTHFDYW |
| | CDR3 | |
| 278 | PMX051 light chain | GGDIIGSKSVY |
| | CDR1 | |
| 279 | PMX051 light chain | RDTNRPT |
| | CDR2 | |
| 280 | PMX051 light chain | CQVWDSSTQAVF |
| | CDR3 | |
| 281 | PMX052 Heavy chain | GAGGTGCAGCTGGTGGAGTCTGGGGGAGACCTGGTGAAG |
| | variable region nucleic | CCTGCGGGGTCCCTGAGACTGTCCTGTGTGGCCTCTGGAT |
| | acid sequence | TCACCTTCAGTAGCTACAGCATGAGCTGGGTCCGCCAGGC |
| | | TCCTGAGAAGGGGCTGCAGTTGGTCGCAGGTATTAGCGGC |
| | | GGTGGAAGTCGCACATACTACTCAGACGCTGTGAAGGGCC |
| | | GATTCACCATCTCCAGAGACAACGCCAAGAACACAGTGTAT |
| | | CTGCAGATGAACAGCCTGAGAGCCGAGGACACGGCCATTT |
| | | ATTACTGTGCAAAGGATGGTGGCAGCTGGGCGGGATATAA |
| | | TTTTGACTACTGGGGCCAGGGAACCCTGGTCACCGTCTCC |
| | | TCAG |
| 282 | PMX052 Heavy chain | EVQLVESGGDLVKPAGSLRLSCVASGFTFSSYSMSWVRQAP |
| | variable region amino | EKGLQLVAGISGGGSRTYYSDAVKGRFTISRDNAKNTVYLQM |
| | acid sequence | NSLRAEDTAIYYCAKDGGSWAGYNFDYWGQGTLVTVSS |
| 283 | PMX052 light chain | GATATTGTCATGACACAGACGCCACCGTCCCTGTCTGTCA |
| | variable region | GCCCTAGAGAGACGGCCTCCATCTCCTGCAAGGCCAGTCA |
| | nucleotide sequence | GAGCCTCCTGCACAGTGATGGAAGCACCTATTTGGATTGG |
| | | TACCTGCAAAAGCCAGGCCAGTCTCCACAGCTTCTGATATA |
| | | CTTGGGTTCCAACCGCTTCACTGGCGTGTCAGACAGGTTC |
| | | AGTGGCAGCGGGTCAGGGACAGAATTCACCCTGAGAATCA |
| | | GCAGAGTGGAGGCTAACGATACTGGAGTTTATTACTGCGG |
| | | GCAAGGTACACAGCTTCCTTATACTTTCGGCCAGGGAACC |
| | | GAGCTGGAGATAAAAC |
| 284 | PMX052 light chain | DIVMTQTPPSLSVSPRETASISCKASQSLLHSDGSTYLDWYLQ |
| | variable region amino | KPGQSPQLLIYLGSNRFTGVSDRFSGSGSGTEFTLRISRVEAN |
| | acid sequence | DTGVYYCGQGTQLPYTFGQGTELEIK |
| 285 | PMX052 heavy chain | GFTFSSY |
| | CDR1 | |
| 286 | PMX052 heavy chain | SGGGSR |
| | CDR2 | |
| 287 | PMX052 heavy chain | CAKDGGSWAGYNFDYW |
| | CDR3 | |
| 288 | PMX052 light chain | KASQSLLHSDGSTYLD |
| | CDR1 | |
| 289 | PMX052 light chain | LGSNRFT |
| | CDR2 | |
| 290 | PMX052 light chain | CGQGTQLPYTF |
| | CDR3 | |
| 291 | PMX053 Heavy chain | GAGGTGCAGCTGGTGGAGTCTGGGGGAGACCTGGTGAAG |
| | variable region nucleic | CCTGCGGGGTCCCTAAGACTGTCCTGTGTGGCCTCTGGTT |
| | acid sequence | TCACCTTCAGTAGCTACAGCATGAGCTGGGTCCGCCAGGC |
| | | TCCTGAGAAGGGGCTGCAGTTGGTCGCAGGTATTAACAGC |
| | | GCTGGAAGTAGCACATACTACACAGACGCTGTGAAGGGCC |
| | | GATTCACCATATCCAGAGACAACGCCAAGAACACAGTGTAT |
| | | TTGCAGATGAACAGCCTGAGAGCCGAGGACACGGCCATTT |
| | | ATTACTGTGCAAAGCGTTGGGTTTTTGACTACTGGGGCCA |
| | | GGGAACCCTGGTCACCGTCTCCTCAG |
| 292 | PMX053 Heavy chain | EVQLVESGGDLVKPAGSLRLSCVASGFTFSSYSMSWVRQAP |
| | variable region amino | EKGLQLVAGINSAGSSTYYTDAVKGRFTISRDNAKNTVYLQM |
| | acid sequence | NSLRAEDTAIYYCAKRWVFDYWGQGTLVTVSS |
| 293 | PMX053 light chain | GATATTGTCATGACACAGACCCCACTGTCCCTGTCCGTCA |
| | variable region | GCCCTGGAGAGCCGGCCTCCATCTCCTGCAAGGCCAGTC |
| | nucleotide sequence | AGAGCCTCCCGCACAGTCTTGGGAACACCTATTTATACTG |
| | | GTTCCGACAGAAGCCAGGCCAGTCTCCACAGCGTTTGATC |
| | | TATAAGGTCTCCAACAGAGACCCTGGGGTCCCAGACAGGT |
| | | TCAGTGGCAGCGGGTCAGGGACAGATTTCACCCTGAGAAT |
| | | CAGCAGAGTGGAGGCTGATGATGCTGGAATTTATTACTGC |
| | | GGGCAAGGTATACAAGATCCGTTCACTTTTGGCCAAGGGA |
| | | CCAAACTGGAGATCAAAC |
| 294 | PMX053 light chain | DIVMTQTPLSLSVSPGEPASISCKASQSLPHSLGNTYLYWFRQ |
| | variable region amino | KPGQSPQRLIYKVSNRDPGVPDRFSGSGSGTDFTLRISRVEA |
| | acid sequence | DDAGIYYCGQGIQDPFTFGQGTKLEIK |
| 295 | PMX053 heavy chain | GFTFSSY |
| | CDR1 | |
| 296 | PMX053 heavy chain | NSAGSS |
| | CDR2 | |
| 297 | PMX053 heavy chain | CAKRWVFDYW |
| | CDR3 | |
| 298 | PMX053 light chain | KASQSLPHSLGNTYLY |
| | CDR1 | |
| 299 | PMX053 light chain | KVSNRDP |
| | CDR2 | |
| 300 | PMX053 light chain | CGQGIQDPFTF |
| | CDR3 | |
| 301 | PMX054 Heavy chain | GAGGTGCACCTGGTGGAGTCTGGGGGAGACCTTGTGAAA |
| | variable region nucleic | CCTGAGGGGTCCCTGAGACTCTCCTGTGTGGTCTCTGGCT |
| | acid sequence | TCACCTTCAGTAGCTACGACATGAGCTGGGTCCGCCAGGC |
| | | TCCAGGGAAGGGGCTGCAGTGGGTCGCATACATTAGCAGT |
| | | GATGGAAGGAGCACAGGTTACACAGACGCTGTGAAGGGC |
| | | CGATTCACCATCTCCAGAGACAATGCCAAGAAAACGCTGTA |
| | | TCTGCAGATGAACAGCCTGAGAAGTGAGGACACAGCCGTT |
| | | TATTACTGTGCGAAGGAGACGCCTAGGTGGGGCTTTGACT |
| | | ACTGGGGCCAGGGAACCCTGGTCACCGTCTCCTCAG |
| 302 | PMX054 Heavy chain | EVHLVESGGDLVKPEGSLRLSCVVSGFTFSSYDMSWVRQAP |
| | variable region amino | GKGLQWVAYISSDGRSTGYTDAVKGRFTISRDNAKKTLYLQM |
| | acid sequence | NSLRSEDTAVYYCAKETPRWGFDYWGQGTLVTVSS |
| 303 | PMX054 light chain | TCCTATGTGCTGACACAGCTGCCATCCGTGAGTGTGACCC |
| | variable region | TGAGGCAGACGGCCCGCATCACCTGTGGGGGAGCCAGTA |
| | nucleotide sequence | TTGGAAGTAAAAATGTTTACTGGCACCAGCAGAAGCTGGG |
| | | CCAGGCCCCTTTACTGATTATCTATGATGATAGCAGCAGGC |
| | | CGTCAGGGATCCCTGAGCGATTCTCCGGCGCCAACTCGG |
| | | GGAACACGGCCACCCTGACCATCAGCGGGGCCCTGGCCG |
| | | AAGATGAGGCTGACTATTACTGCCAGGTGTGGGACAGCAG |
| | | TACTAGTGTGTTCGGCGGAGGCACCCATCTGACCGTCCTC |
| | | G |
| 304 | PMX054 light chain | SYVLTQLPSVSVTLRQTARITCGGASIGSKNVYWHQQKLGQA |
| | variable region amino | PLLIIYDDSSRPSGIPERFSGANSGNTATLTISGALAEDEADYY |
| | acid sequence | CQVWDSSTSVFGGGTHLTVL |
| 305 | PMX054 heavy chain | GFTFSSY |
| | CDR1 | |
| 306 | PMX054 heavy chain | SSDGRS |
| | CDR2 | |
| 307 | PMX054 heavy chain | CAKETPRWGFDYW |
| | CDR3 | |
| 308 | PMX054 light chain | GGASIGSKNVY |
| | CDR1 | |
| 309 | PMX054 light chain | DDSSRPS |
| | CDR2 | |
| 310 | PMX054 light chain | CQVWDSSTSVF |
| | CDR3 | |
| 311 | PMX055 Heavy chain | GAGGTGCAGCTGGAGGAGTCTGGGGGAGACCTTGTGAAA |
| | variable region nucleic | CCTGAGGGGTCCCTGAGACTCTCCTGTGTGGTCTCTGGCT |
| | acid sequence | TCACCCTCAGTACCTTCGGCATGAGCTGGGTCCGCCAGGC |
| | | TCCAGGGAAGGGGCTGCAGTGGGTCGCATACATTAGCAGT |
| | | GATGGAAGGAGCACAAGTTACACAGACGCTGTGAAGGGCC |
| | | GATTCACCATCTCCAGAGACAATGCCAAGAACACGCTGTAT |
| | | CTGCAGATGAACAGCCTGCGAACTGACGACACAGCCGTGT |
| | | ATTACTGTGTCCAGTTGGGCCGGGGATTTGGTATAGACTA |
| | | CTGGGGCCATGGCACCTCACTCTTCGTGTCCTCAG |
| 312 | PMX055 Heavy chain | EVQLEESGGDLVKPEGSLRLSCVVSGFTLSTFGMSWVRQAP |
| | variable region amino | GKGLQWVAYISSDGRSTSYTDAVKGRFTISRDNAKNTLYLQM |
| | acid sequence | NSLRTDDTAVYYCVQLGRGFGIDYWGHGTSLFVSS |
| 313 | PMX055 light chain | TCCTATGTGCTGACACAGCTGCCATCCAAAAATGTGACCCT |
| | variable region | GAGGCAGCCGGCCCACATCCCCTGTGGGGGAGACAACAT |
| | nucleotide sequence | TGGAAGTAAAAGTGTTCACTGGTACCAGCAGAAGCTGGGC |
| | | CAGGCCCCTGTACTGATTATCTATTATGATACCAGCAGGCC |
| | | GACAGGGATCCCTGAGCGATTCTCCGGCGCCAACTCGGG |
| | | GAACACGGCCACCCTGACCATCAGCGGGGCCCTGGCCGA |
| | | GGACGAGGCTGACTATTACTGCCAGGTGTGGGACAGCAGT |
| | | GCTGCTGTGTTCGGCGGAGGCACCCACCTGACCGTCCTC |
| | | G |
| 314 | PMX055 light chain | SYVLTQLPSKNVTLRQPAHIPCGGDNIGSKSVHWYQQKLGQA |
| | variable region amino | PVLIIYYDTSRPTGIPERFSGANSGNTATLTISGALAEDEADYY |
| | acid sequence | CQVWDSSAAVFGGGTHLTVL |
| 315 | PMX055 heavy chain | GFTLSTF |
| | CDR1 | |
| 316 | PMX055 heavy chain | SSDGRS |
| | CDR2 | |
| 317 | PMX055 heavy chain | CVQLGRGFGIDYW |
| | CDR3 | |
| 318 | PMX055 light chain | GGDNIGSKSVH |
| | CDR1 | |
| 319 | PMX055 light chain | YDTSRPT |
| | CDR2 | |
| 320 | PMX055 light chain | CQVWDSSAAVF |
| | CDR3 | |
| 321 | PMX056 Heavy chain | GAGGTGCAGCTGGTGGAGTCTGGGGGAGACCTTGTGAAA |
| | variable region nucleic | CCTGAGGGGTCCCTGAGACTCTCCTGTGTGGTCTCTGGCT |
| | acid sequence | TCACCTTGAGTAGCTACGGCATGAGCTGGGTCCGCCAGGC |
| | | TCCAGGGAGGGGGCTGCAGTGGGTCGCATACATTAGCAG |
| | | TGATGGAAGGAGCACAAGTTACACAGACGCTGTGAAGGGC |
| | | CGATTCACCATCTCCAGAGACAATGCCAAGAACACGCTGT |
| | | ATCTGCAGATGAACAGCCTGAGAAGTGAGGACACAGCCGT |
| | | GTATTACTGTGTCCAGTTGGGCCGGGGCTTTGGCATGGAC |
| | | TATTGGGGCCATGGCACCTCACTCTTCGTGTCCTCAG |
| 322 | PMX056 Heavy chain | EVQLVESGGDLVKPEGSLRLSCVVSGFTLSSYGMSWVRQAP |
| | variable region amino | GRGLQWVAYISSDGRSTSYTDAVKGRFTISRDNAKNTLYLQM |
| | acid sequence | NSLRSEDTAVYYCVQLGRGFGMDYWGHGTSLFVSS |
| 323 | PMX056 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGCGTTCACTGGTACCAGCAGAAGCTGGGCC |
| | | AGGCCCCTGTAGTGATTATCTATTATGATAGTACCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTGCTGTGTTCGGCGGAGGCACCCACCTGACCGTCCTCG |
| 324 | PMX056 light chain | SYVLTQLPSKNVTLKQPAHITCGGDNIGSKSVHWYQQKLGQA |
| | variable region amino | PVVIIYYDSTRPTGIPERFSGANSGNTATLTISGALAEDEADYY |
| | acid sequence | CQVWDSSAAVFGGGTHLTVL |
| 325 | PMX056 heavy chain | GFTLSSY |
| | CDR1 | |
| 326 | PMX056 heavy chain | SSDGRS |
| | CDR2 | |
| 327 | PMX056 heavy chain | CVQLGRGFGMDYW |
| | CDR3 | |
| 328 | PMX056 light chain | GGDNIGSKSVH |
| | CDR1 | |
| 329 | PMX056 chain | YDSTRPT |
| | CDR2 | |
| 330 | PMX056 light chain | CQVWDSSAAVF |
| | CDR3 | |
| 331 | PMX057 Heavy chain | GAGGTGCAACTGGTGGAGTCTGGGGGAGACCTTGTGAAA |
| | variable region nucleic | CCTGAGGGGTCCCTGAGACTCTCCTGTGTGGTCTCTGGCT |
| | acid sequence | TCACCTTCAATAACTACGGCATGAGTTGGGTCCGCCAGGC |
| | | TCCAGGGAAGGGGCTGCAGTGGGTCGCATACATTAGTATT |
| | | GATGGAAGGAGCACAAGTTACACAGACGCTGTGAAGGGCC |
| | | GATTCACCATCTCCAGAGACAATGCCAAGAACACGCTGTAT |
| | | CTGCATATAAACAGCCTGAGAACTGAGGACACAGCCGTGT |
| | | ATTACTGTACGAAGTTGGGGAGGGGGTTCACTTTTGGTTAC |
| | | TGGGGCCAGGGCACCCTGGTCACTGTCTCCTCAG |
| 332 | PMX057 Heavy chain | EVQLVESGGDLVKPEGSLRLSCVVSGFTFNNYGMSWVRQAP |
| | variable region amino | GKGLQWVAYISIDGRSTSYTDAVKGRFTISRDNAKNTLYLHIN |
| | acid sequence | SLRTEDTAVYYCTKLGRGFTFGYWGQGTLVTVSS |
| 333 | PMX057 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGCACTGATTATCTATTATGATAGCAGCAGGCC |
| | | GACAGGGATCCCTGAGCGATTCTCCGGCGCCAACTCGGG |
| | | GAACACGGCCACCCTGACCATCAGCGGGGCCCTGGCCGA |
| | | GGACGAGGCTGACTATTACTGCCAGGTGTGGGACAGCAGT |
| | | GTTAGTGTGTTCGGCGGAGGCACCCATCTGACCGTCCTCG |
| 334 | PMX057 light chain | SYVLTQLPSKNVTLKQPAHITCGGDNIGSKSVHWYQQKLGQA |
| | variable region amino | PALIIYYDSSRPTGIPERFSGANSGNTATLTISGALAEDEADYY |
| | acid sequence | CQVWDSSVSVFGGGTHLTVL |
| 335 | PMX057 heavy chain | GFTFNNY |
| | CDR1 | |
| 336 | PMX057 heavy chain | SIDGRS |
| | CDR2 | |
| 337 | PMX057 heavy chain | CTKLGRGFTFGYW |
| | CDR3 | |
| 338 | PMX057 light chain | GGDNIGSKSVH |
| | CDR1 | |
| 339 | PMX057 light chain | YDSSRPT |
| | CDR2 | |
| 340 | PMX057 light chain | CQVWDSSVSVF |
| | CDR3 | |
| 341 | PMX058 Heavy chain | GAGGTGCAGCTGGTGGAGTCTGGGGGAGACCTTGTGAAA |
| | variable region nucleic | CCTGAGGGGTCCCTGAGACTCTCCTGTGTGGTCTCTGACT |
| | acid sequence | TCACCTTCGGTAGCTACGGCATGAGCTGGGTCCGCCAGGC |
| | | TCCAGGGAAGGGGCTGCAGTGGGTCGCATACATTAGCAGT |
| | | GATGGAAGGAGCACAAGTTACACAGACGCTGTGAAGGGCC |
| | | GATTCACCATCTCCAGAGACAATGCCAAGAACACGCTACAT |
| | | CTGCAGATGAACAGCCTGAGAACTGAGGACACAGCCGTGT |
| | | ATTACTGTGCGAAGTTGGGGAGGGGGTTTGCTTTTGGTTA |
| | | CTGGGGCCAGGGCACCCTGGTCACTGTCTCCTCAG |
| 342 | PMX058 Heavy chain | EVQLVESGGDLVKPEGSLRLSCVVSDFTFGSYGMSWVRQAP |
| | variable region amino | GKGLQWVAYISSDGRSTSYTDAVKGRFTISRDNAKNTLHLQM |
| | acid sequence | NSLRTEDTAVYYCAKLGRGFAFGYWGQGTLVTVSS |
| 343 | PMX058 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTACTGATTATCTATTATGATAGCAACAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAGTGTGTTCGGCGGAGGCACCCATCTGACCGTCCTCG |
| 344 | PMX058 light chain | SYVLTQLPSKNVTLKQPAHITCGGDNIGSKSVHWYQQKLGQA |
| | variable region amino | PVLIIYYDSNRPTGIPERFSGANSGNTATLTISGALAEDEADYY |
| | acid sequence | CQVWDSSASVFGGGTHLTVL |
| 345 | PMX058 heavy chain | DFTFGSY |
| | CDR1 | |
| 346 | PMX058 heavy chain | SSDGRS |
| | CDR2 | |
| 347 | PMX058 heavy chain | CAKLGRGFAFGYW |
| | CDR3 | |
| 348 | PMX058 light chain | GGDNIGSKSVH |
| | CDR1 | |
| 349 | PMX058 light chain | YDSNRPT |
| | CDR2 | |
| 350 | PMX058 light chain | CQVWDSSASVF |
| | CDR3 | |
| 351 | PMX059 Heavy chain | GAGGTTCACCTGGTGGAGTCTGGGGGAGACCTTGTGAAAC |
| | variable region nucleic | CTGAGGGGTCCCTGAGACTCTCCTGTGTGGTCTCTGGCTT |
| | acid sequence | CACCTTCAGTAACTACGTCATGAACTGGGTCCGCCAGGCT |
| | | CCAGGGAAGGGGCTGCAGTGGGTCGCATTCATTAGCGGT |
| | | GATGGAAGGAGCACAACTTACACAGACGCTGTGAAGAGCC |
| | | GATTCACCATCTCCAGAGACAATGCCAAGAACACGCTGTAT |
| | | CTGCAGATGAGCAGCCTGAGAACTGAGGACACAGCCATAT |
| | | ACTTCTGTGCGAAGGAGAAATATAGTAGCGGCTGGTCTTTT |
| | | GAGTACTGGGGCCAGGGAACCCTGGTCACCGTCTCCTCA |
| | | G |
| 352 | PMX059 Heavy chain | EVHLVESGGDLVKPEGSLRLSCVVSGFTFSNYVMNWVRQAP |
| | variable region amino | GKGLQWVAFISGDGRSTTYTDAVKSRFTISRDNAKNTLYLQM |
| | acid sequence | SSLRTEDTAIYFCAKEKYSSGWSFEYWGQGTLVTVSS |
| 353 | PMX059 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTATTGATTATCTATTATGACAGCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAGTGTGTTCGGCGGAGGCACCCATCTGACCGTCCTCG |
| 354 | PMX059 light chain | SYVLTQLPSKNVTLKQPAHITCGGDNIGSKSVHWYQQKLGQA |
| | variable region amino | PVLIIYYDSSRPTGIPERFSGANSGNTATLTISGALAEDEADYY |
| | acid sequence | CQVWDSSASVFGGGTHLTVL |
| 355 | PMX059 heavy chain | GFTFSNY |
| | CDR1 | |
| 356 | PMX059 heavy chain | SGDGRS |
| | CDR2 | |
| 357 | PMX059 heavy chain | CAKEKYSSGWSFEYW |
| | CDR3 | |
| 358 | PMX059 light chain | GGDNIGSKSVH |
| | CDR1 | |
| 359 | PMX059 light chain | YDSSRPT |
| | CDR2 | |
| 360 | PMX059 light chain | CQVWDSSASVF |
| | CDR3 | |
| 361 | PMX060 Heavy chain | GAGGTGCAGCTGGTGGAGTCTGGGGGAGACCTGATGAAG |
| | variable region nucleic | CCTGGGGGGTCCCTGAGACTTTCCTGTGTGGCCTCTGGAT |
| | acid sequence | TCACCTTCAGTAGCTTCCCCATGAGTTGGGTCCGCCAGGC |
| | | TCCAGGGAAGGGGCTTCAGTGGGTCGCATACATTCACAGT |
| | | GGTGGAAGTAGCACAAGCTATGCAGACGCTGTGAAGGGC |
| | | CGATTCACCATCTCCAGAGACAACGCCAAGAATACGCTGT |
| | | ATCTTCAGATGAACAGCCTGCGAGCCGAGGACACGGCCAT |
| | | ATATTACTGTGCGCGTACCTTCCCTACGGTAGCTGCTGACA |
| | | ACTGGTTCTACTACTGGGGCCAAGGGACCCTGGTCACTGT |
| | | GTCCTCAG |
| 362 | PMX060 Heavy chain | EVQLVESGGDLMKPGGSLRLSCVASGFTFSSFPMSWVRQAP |
| | variable region amino | GKGLQWVAYIHSGGSSTSYADAVKGRFTISRDNAKNTLYLQM |
| | acid sequence | NSLRAEDTAIYYCARTFPTVAADNWFYYWGQGTLVTVSS |
| 363 | PMX060 light chain | TCCTATGTGCTGACACAGCTGCCATCCAAAAATGTGACCCT |
| | variable region | GAAGCAGGCGGCCCACATCACCTGTGGGGGAGACAACAT |
| | nucleotide sequence | TGGAAGTAAAAGTGTTCACTGGTACCAGCAGAAGCTGGGC |
| | | CAGGCCCCTGTACTGATTATCTATTATGATAGCAGCAGGCC |
| | | GACAGGGATCCCTGAGCGATTCTCCGGCGCCAACTCGGG |
| | | GAACACGGCCACCCTGACCATCAGCGGGGCCCTGGCCGA |
| | | GGACGAGGCTGACTATTACTGCCAGGTGTGGGACAGCAGT |
| | | GCTAAGTGGGTATTCGGTGAAGGGACCCAGCTGACCGTCC |
| | | TCG |
| 364 | PMX060 light chain | SYVLTQLPSKNVTLKQAAHITCGGDNIGSKSVHWYQQKLGQA |
| | variable region amino | PVLIIYYDSSRPTGIPERFSGANSGNTATLTISGALAEDEADYY |
| | acid sequence | CQVWDSSAKWVFGEGTQLTVL |
| 365 | PMX060 heavy chain | GFTFSSF |
| | CDR1 | |
| 366 | PMX060 heavy chain | HSGGSS |
| | CDR2 | |
| 367 | PMX060 heavy chain | CARTFPTVAADNWFYYW |
| | CDR3 | |
| 368 | PMX060 light chain | GGDNIGSKSVH |
| | CDR1 | |
| 369 | PMX060 light chain | YDSSRPT |
| | CDR2 | |
| 370 | PMX060 light chain | CQVWDSSAKWVF |
| | CDR3 | |
| 371 | PMX061 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAGTTTCCCCATGAGCTGGGTCCGCCAGGC |
| | | TCCAGGGAAGGGGCTTCAGTGGGTCGCATACATTAACAGT |
| | | GGTGGAAGTAGCACAAGTTATGCAGACGTTGTGAAGGGCC |
| | | GATTCACCATCTCCAGAGACAACGCCAAGAACACGCTGTA |
| | | TCTTCAGATGAACAGCCTGAGAGCCGACGACACGGCCGTA |
| | | TATTACTGTGCGCGTACCTTCCCTACGGTAGCTACTGACAA |
| | | CTGGTTCTACTACTGGGGCCAAGGGACCCTGGTCACTGTG |
| | | TCCTCAG |
| 372 | PMX061 Heavy chain | EVQLVESGGDLVKPGGSLRLSCVASGFTFSSFPMSWVRQAP |
| | variable region amino | GKGLQWVAYINSGGSSTSYADVVKGRFTISRDNAKNTLYLQM |
| | acid sequence | NSLRADDTAVYYCARTFPTVATDNWFYYWGQGTLVTVSS |
| 373 | PMX061 light chain | TCCTATGTGCTGACACAGCTGCCATCCAAAAATGTGACCCT |
| | variable region | GAAGCAGGCGGCCCACATCACCTGTGGGGGAGACAACAT |
| | nucleotide sequence | TGGAAGTAAAAGTGTTCACTGGTACCAGCAGAAGCTGGGC |
| | | CAGGCCCCTGTACTGATTATCTATTATGATAGCAGCAGGCC |
| | | GACAGGGATCCCTGAGCGATTCTCCGGCGCCAACTCGGG |
| | | GAACACGGCCACCCTGACCATCAGCGGGGCCCTGGCCGA |
| | | GGACGAGGCTGACTATTACTGCCAGGTGTGGGACAGCAGT |
| | | GCTAAGTGGGTATTCGGTGAAGGGACCCAGCTGACCGTCC |
| | | TCG |
| 374 | PMX061 light chain | SYVLTQLPSKNVTLKQAAHITCGGDNIGSKSVHWYQQKLGQA |
| | variable region amino | PVLIIYYDSSRPTGIPERFSGANSGNTATLTISGALAEDEADYY |
| | acid sequence | CQVWDSSAKWVFGEGTQLTVL |
| 375 | PMX061 heavy chain | GFTFSSF |
| | CDR1 | |
| 376 | PMX061 heavy chain | NSGGSS |
| | CDR2 | |
| 377 | PMX061 heavy chain | CARTFPTVATDNWFYYW |
| | CDR3 | |
| 378 | PMX061 light chain | GGDNIGSKSVH |
| | CDR1 | |
| 379 | PMX061 light chain | YDSSRPT |
| | CDR2 | |
| 380 | PMX061 light chain | CQVWDSSAKWVF |
| | CDR3 | |
| 381 | PMX062 Heavy chain | GAGGTGCAGTTGGTGGAGTCTGGGGGAGACCTTGTGAAA |
| | variable region nucleic | CCTGAGGGGTCCCTGAGACTCTCCTGTGTGGTCTCTGGCT |
| | acid sequence | TCACCTTCAGTAGTTATGACATGAGCTGGGTCCGCCAGGC |
| | | TCCAGGGAAGGGGCTGCAGTGGGTCGCATACATTAGCTTT |
| | | GATGGAAGGAGCACAAGTTACACAGACGTTGTGAAGGGCC |
| | | GATTCACCATCTCCAGAGACAATGCCAAGACCACGCTGTAT |
| | | CTGCAGATGAACACCCTGAGAATTGAGGACACAGCCGTGT |
| | | ATTACTGTGCGAAGTTAAGTTCTAGTGGTTGGAGTATGGAC |
| | | TATTGGGGCCATGGCACCTCACTCTTCGTGTCCTCAG |
| 382 | PMX062 Heavy chain | EVQLVESGGDLVKPEGSLRLSCVVSGFTFSSYDMSWVRQAP |
| | variable region amino | GKGLQWVAYISFDGRSTSYTDVVKGRFTISRDNAKTTLYLQM |
| | acid sequence | NTLRIEDTAVYYCAKLSSSGWSMDYWGHGTSLFVSS |
| 383 | PMX062 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTACTGATTATCTATTATGATAGCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAGTGTGTTCGGCGGAGGCACCCATCTGACCGTCCTCG |
| 384 | PMX062 light chain | SYVLTQLPSKNVTLKQPAHITCGGDNIGSKSVHWYQQKLGQA |
| | variable region amino | PVLIIYYDSSRPTGIPERFSGANSGNTATLTISGALAEDEADYY |
| | acid sequence | CQVWDSSASVFGGGTHLTVL |
| 385 | PMX062 heavy chain | GFTFSSY |
| | CDR1 | |
| 386 | PMX062 heavy chain | SFDGRS |
| | CDR2 | |
| 387 | PMX062 heavy chain | CAKLSSSGWSMDYW |
| | CDR3 | |
| 388 | PMX062 light chain | GGDNIGSKSVH |
| | CDR1 | |
| 389 | PMX062 light chain | YDSSRPT |
| | CDR2 | |
| 390 | PMX062 light chain | CQVWDSSASVF |
| | CDR3 | |
| 391 | PMX063 Heavy chain | GAGGTACAACTGGTGGAGTCTGGGGGAGACCTGGTGAGG |
| | variable region nucleic | CCTGGGGGGTCCCTGAGACTTTCCTGTGTGGCCTCTGGAT |
| | acid sequence | TCACCTTCAATAATTACCACATGAGCTGGGTCCGCCAGGCT |
| | | CCTGGGAAGGGGCTTCAGTGGGTCGCATTCATTAACAGTG |
| | | GTGGAAGTGGTACAACCTTTGCAGACGTTGTGAGGGGCCG |
| | | ATTCACCATCTCCAGAGACTACGCCAAAAACGCCCTGTATC |
| | | TTCAGATGAACAGCCTGAGAGCCGAGGACACGGCCGTATA |
| | | TTATTGTGCGAGTGGATCTATATGGACTCATTTTGACTACT |
| | | GGGGCCAGGGAACCCTGGTCACCGTCTCCTCAG |
| 392 | PMX063 Heavy chain | EVQLVESGGDLVRPGGSLRLSCVASGFTFNNYHMSWVRQAP |
| | variable region amino | GKGLQWVAFINSGGSGTTFADVVRGRFTISRDYAKNALYLQM |
| | acid sequence | NSLRAEDTAVYYCASGSIWTHFDYWGQGTLVTVSS |
| 393 | PMX063 light chain | TCCTTTGTGCTGACACAGCTGCCATCCGTGAGTGTGACCC |
| | variable region | TGAGGCAGACGGCCCGCATCACCTGTGGGGGAGACATCA |
| | nucleotide sequence | TTGGAAGTAAAAATGTTTACTGGTACCAGCAGAGGCTGGG |
| | | CCAGGCCCCTGTATTGATTATCTATAGAGATAACAACAGGC |
| | | CGACAGGGATCCCTGAGCGATTCTCTGGCGCCAGCTCGG |
| | | GGAACACGGCCACCCTGACCATCAGCGGGGCCCTGGCCG |
| | | AGGACGAGGCTGACTATTACTGCCAGGTGTGGGACAACAG |
| | | TAATAAGGCTATGTTCGGCGGAGGCACCCATCTGACCGTC |
| | | CTCG |
| 394 | PMX063 light chain | SFVLTQLPSVSVTLRQTARITCGGDIIGSKNVYWYQQRLGQAP |
| | variable region amino | VLIIYRDNNRPTGIPERFSGASSGNTATLTISGALAEDEADYYC |
| | acid sequence | QVWDNSNKAMFGGGTHLTVL |
| 395 | PMX063 heavy chain | GFTFNNY |
| | CDR1 | |
| 396 | PMX063 heavy chain | NSGGSG |
| | CDR2 | |
| 397 | PMX063 heavy chain | CASGSIWTHFDYW |
| | CDR3 | |
| 398 | PMX063 light chain | GGDIIGSKNVY |
| | CDR1 | |
| 399 | PMX063 light chain | RDNNRPT |
| | CDR2 | |
| 400 | PMX063 light chain | CQVWDNSNKAMF |
| | CDR3 | |
| 401 | PMX064 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAGTTATCACATGAGCTGGGTCCGCCAGGC |
| | | TCCAGGGAAGGGGCTTCAGTGGGTCGCATATATTACCAGT |
| | | GGTGGGATTACCACAAGCTATGCAGACGCTGTGAAGGGCC |
| | | GATTCACCATCTCCAGAGACAACGCCAAGAACACGCTGTA |
| | | TCTTCAGATGAACAGCCTGAGAGCCGAGGACACGGCCGTG |
| | | TATTACTGTGCGAGTGATCTGGTTCTAATATTAGGGGGGGC |
| | | TGACTACTGGGGCCAGGGAACCCTGGTCACCGTCTCCTCA |
| | | G |
| 402 | PMX064 Heavy chain | EVQLVESGGDLVKPGGSLRLSCVASGFTFSSYHMSWVRQAP |
| | variable region amino | GKGLQWVAYITSGGITTSYADAVKGRFTISRDNAKNTLYLQM |
| | acid sequence | NSLRAEDTAVYYCASDLVLILGGADYWGQGTLVTVSS |
| 403 | PMX064 light chain | TCCTATGAGCTGACTCAGCCACCATCCGTGAATGTGACCC |
| | variable region | TGAGTGAGACGGCCCACATCACCTGTGGGGGAGACAGCA |
| | | TTGGAAGGAAATATGTTCAATGGATCCAGCAGAATCCAGG |
| | nucleotide sequence | CCAGGCCCCCGTGGTGATTATCTATAAAGATAGCAACAGG |
| | | CCGACAGGGATCCCTGAGCGCTTCTCTGGCGCCAACTCAG |
| | | GGAACACGGCTACCCTGACCATCAGTGGGGCCCTGGCCG |
| | | AAGACGAGGCTGACTATTACTGCCAGGTGGGGGACAGTG |
| | | GTACTAAGGCTGTGTTCGGCGGAGGCACCCACCTGACCGT |
| | | CCTCG |
| 404 | PMX064 light chain | SYELTQPPSVNVTLSETAHITCGGDSIGRKYVQWIQQNPGQA |
| | variable region amino | PVVIIYKDSNRPTGIPERFSGANSGNTATLTISGALAEDEADYY |
| | acid sequence | CQVGDSGTKAVFGGGTHLTVL |
| 405 | PMX064 heavy chain | GFTFSSY |
| | CDR1 | |
| 406 | PMX064 heavy chain | TSGGIT |
| | CDR2 | |
| 407 | PMX064 heavy chain | CASDLVLILGGADYW |
| | CDR3 | |
| 408 | PMX064 light chain | GGDSIGRKYVQ |
| | CDR1 | |
| 409 | PMX064 light chain | KDSNRPT |
| | CDR2 | |
| 410 | PMX064 light chain | CQVGDSGTKAVF |
| | CDR3 | |
| 411 | PMX065 Heavy chain | GAGGTGCAGCTGGTGGAGTCTGGGGGAGACCTGGTGAAG |
| | variable region nucleic | CCTGGGGGGTCCCTGAGACTCTCCTGTGTGGCCTCTGGAT |
| | acid sequence | TCACCTTCAATAACTACGAAATGTACTGGGTCCGCCAGGCT |
| | | CCAGGGAAAGGGCTGGAGTGGGTCGCAAGGATTTATGAG |
| | | AGTGGAAGTACCACTTTTTCTGCAGAAGCTGTTACGGGCC |
| | | GATTCACCATCTCCAGAGACAACGCCAAGAACATGGTGTAT |
| | | CTGCAGATGAACAGCCTGAGAGCCGAGGACACGGCCGTG |
| | | TATTACTGTGCGAGTGCTAGTTCTAGTTCTTGGCGTTTTGA |
| | | CTACTGGGGCCAGGGAACCCTGGTCACCGTCTCCTCAG |
| 412 | PMX065 Heavy chain | EVQLVESGGDLVKPGGSLRLSCVASGFTFNNYEMYWVRQAP |
| | variable region amino | GKGLEWVARIYESGSTTFSAEAVTGRFTISRDNAKNMVYLQM |
| | acid sequence | NSLRAEDTAVYYCASASSSSWRFDYWGQGTLVTVSS |
| 413 | PMX065 light chain | TCCTATGTGCTGACACAGCTGCCATCCGTGAGTGTGACCC |
| | variable region | TGGGGCAGACGGCCCGCATCACCTGTGGGGGAGACAGCA |
| | nucleotide sequence | TTGGGCGAAAAAATGTTTGCTGGTTCCAGCAGAGGCTGGG |
| | | CCAGGCCCCTGAACTGATTATCTATGATGATCGCAGCAGG |
| | | CCGTCAGGGATCCCTGAGCGATTCTCCGGCGCCAACTCG |
| | | GGGAACACGGCCACCCTGACCATCAGCGGGGCCCTGGCC |
| | | GAGGATGAGGCTGACTATTACTGCCAGGTGTGGGACAGCA |
| | | GTACTGGTGTGTTCGGCGGAGGCACCCATCTGACCGTCCT |
| | | CG |
| 414 | PMX065 light chain | SYVLTQLPSVSVTLGQTARITCGGDSIGRKNVCWFQQRLGQA |
| | variable region amino | PELIIYDDRSRPSGIPERFSGANSGNTATLTISGALAEDEADYY |
| | acid sequence | CQVWDSSTGVFGGGTHLTVL |
| 415 | PMX065 heavy chain | GFTFNNY |
| | CDR1 | |
| 416 | PMX065 heavy chain | YESGST |
| | CDR2 | |
| 417 | PMX065 heavy chain | CASASSSSWRFDYW |
| | CDR3 | |
| 418 | PMX065 light chain | GGDSIGRKNVC |
| | CDR1 | |
| 419 | PMX065 light chain | DDRSRPS |
| | CDR2 | |
| 420 | PMX065 light chain | CQVWDSSTGVF |
| | CDR3 | |
| 421 | PMX082 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAGCCACCACATGACCTGGGTCCGCCAGGC |
| | | TCCAGGGAAGGGGCTTCAGTGGGTCGCATATATTGTCAGT |
| | | GGTGGAACTAACACTAACTATGCAGACGCTGTGAAGGGCC |
| | | GGTTCACCATCTCCAGAGACAACGCCAAGAACACGCTGTA |
| | | TCTTCAGATGAACAGCCTGAGAGCCGAGGACACGGCCGTG |
| | | TATTACTGTGCGAGTGATCTAGTTCTAGCAGTTGGGGCTCC |
| | | TTTCTACTGGGGCCAGGGAACCCTGGTCACCGTCTCCTCA |
| | | G |
| 422 | PMX082 Heavy chain | EVQLVESGGDLVKPGGSLRLSCVASGFTFSSHHMTWVRQAP |
| | variable region amino | GKGLQWVAYIVSGGTNTNYADAVKGRFTISRDNAKNTLYLQM |
| | acid sequence | NSLRAEDTAVYYCASDLVLAVGAPFYWGQGTLVTVSS |
| 423 | PMX082 light chain | TCCTATGAGCTGACTCAGCCACCATCCGTGAATGTGACCC |
| | variable region | TGAGGGAGACGGCCCACATCACCTGTGGGGGAGACAGCA |
| | nucleotide sequence | TTGGAAGAAAATATGTTCAATGGATCCAGCAGAATCCAGGC |
| | | CAGGCCCCCGTGGTAATTATCTATAAAGACATCAACAGGC |
| | | CGACAGGGATCCCTGAGCGATTCTCTGGCGCCAACTCAGG |
| | | GAACACGGCTACCCTGACCATCAGTGGGGCCCTGGCCGA |
| | | AGACGAGGCTGACTATTACTGCCAGGTGGGGGACAGTGGT |
| | | ACTAAGGCGGTATTCGGTGAAGGGACCCAGCTGACCGTCC |
| | | TCG |
| 424 | PMX082 light chain | SYELTQPPSVNVTLRETAHITCGGDSIGRKYVQWIQQNPGQA |
| | variable region amino | PVVIIYKDINRPTGIPERFSGANSGNTATLTISGALAEDEADYY |
| | acid sequence | CQVGDSGTKAVFGEGTQLTVL |
| 425 | PMX082 heavy chain | GFTFSSH |
| | CDR1 | |
| 426 | PMX082 heavy chain | VSGGTN |
| | CDR2 | |
| 427 | PMX082 heavy chain | CASDLVLAVGAPFYW |
| | CDR3 | |
| 428 | PMX082 light chain | GGDSIGRKYVQ |
| | CDR1 | |
| 429 | PMX082 light chain | KDINRPT |
| | CDR2 | |
| 430 | PMX082 light chain | CQVGDSGTKAVF |
| | CDR3 | |
| 431 | PMX083 Heavy chain | GAGGTGCAACTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGAACCTACCACATGAGCTGGGTCCGCCAGGC |
| | | TCCAGGGAAGGGGCTTCAGTGGGTCGCATACATTACCCGT |
| | | GATGGAATTAGCACAACCTATACAGACGCTGTGAAGGGCC |
| | | GATTCACCATCTCCAGAGACAACGCCAAGAACACTCTGTTT |
| | | CTTCAGATGAACAGCCTGAGAGCCGATGACACGGCCGTGT |
| | | ATTACTGTGCGAGTGATCTAGTTCTAGTACTTGGGGCTCCT |
| | | TTCTACTGGGGCCAGGGAACCCTGGTCACCGTCTCCTCAG |
| 432 | PMX083 Heavy chain | EVQLVESGGDLVKPGGSLRLSCVASGFTFRTYHMSWVRQAP |
| | variable region amino | GKGLQWVAYITRDGISTTYTDAVKGRFTISRDNAKNTLFLQMN |
| | acid sequence | SLRADDTAVYYCASDLVLVLGAPFYWGQGTLVTVSS |
| 433 | PMX083 light chain | TCCTATGAGCTGACTCAGCCACCATCCGTGAATGTGACCC |
| | variable region | TGGGGGAGACGGCCCACATCACCTGTGGGGGAGACAGCA |
| | nucleotide sequence | TTGGAAGAAAATATGTTCAATGGATCCAGCAGAATCCAGGC |
| | | CAGGCCCCCGTGGTGATTATCTATAAAGATAGCAACAGGC |
| | | CGACAGGGATCCCTGAGCGATTCTCTGGCGCCAACTCAGG |
| | | GAACACGGCTACCCTGACCATCAGTGGGGCCCTGGCCGA |
| | | AGACGAGGCTGACTATTACTGCCAGGTGGGGGACAGTGGT |
| | | ACTAAGGCGGTATTCGGTGAAGGGACCCAGCTGACCGTCC |
| | | TCG |
| 434 | PMX083 light chain | SYELTQPPSVNVTLGETAHITCGGDSIGRKYVQWIQQNPGQA |
| | variable region amino | PVVIIYKDSNRPTGIPERFSGANSGNTATLTISGALAEDEADYY |
| | acid sequence | CQVGDSGTKAVFGEGTQLTVL |
| 435 | PMX083 heavy chain | GFTFRTY |
| | CDR1 | |
| 436 | PMX083 heavy chain | TRDGIS |
| | CDR2 | |
| 437 | PMX083 heavy chain | CASDLVLVLGAPFYW |
| | CDR3 | |
| 438 | PMX083 light chain | GGDSIGRKYVQ |
| | CDR1 | |
| 439 | PMX083 light chain | KDSNRPT |
| | CDR2 | |
| 440 | PMX083 light chain | CQVGDSGTKAVF |
| | CDR3 | |
| 441 | PMX084 Heavy chain | GAGGTGCAGCTGGTGGAGTCTGGGGGAGACCTTGTGAAA |
| | variable region nucleic | CCTGAGGGGTCCCTGAGACTCTCCTGTGTGGTCTCTGGCT |
| | acid sequence | TCACTTTCAGTAATTACGACATGAGCTGGGTCCGCCAGGC |
| | | TCCAGGGAAGGGGCTGCAGTGGGTCGCATTCATTAGCAAT |
| | | GATGGGAGGATCACAGGTTACACAGACGCTGTGGAGGGC |
| | | CGATTCACCATCTCCAGAGACAACGCCAAGAACACGCTGT |
| | | ATCTGCAGATGAACAGCCTGAGAACTGAGGACACAGCCGT |
| | | GTATTACTGTGCGAAGGAGACTAGTCGTTGGGGGTTTGAC |
| | | TACTGGGGCCAGGGAACCCTGGTCACCGTCTCCTCAG |
| 442 | PMX084 Heavy chain | EVQLVESGGDLVKPEGSLRLSCVVSGFTFSNYDMSWVRQAP |
| | variable region amino | GKGLQWVAFISNDGRITGYTDAVEGRFTISRDNAKNTLYLQM |
| | acid sequence | NSLRTEDTAVYYCAKETSRWGFDYWGQGTLVTVSS |
| 443 | PMX084 light chain | TCCTATGTGCTGACACAGCTGCCATCCGTGAGTGTGACCC |
| | variable region | TGAGGCAGACGGCCCGCATCACCTGTGGGGGAGACAGCA |
| | nucleotide sequence | TTGGAAGTAAAAATGTTTACTGGCACCAGCAGAAGCTGGG |
| | | CCAGGCCCCTGTACTGATTATCTATGATGATAGCAGCAGG |
| | | CCGTCAGGGATCCCTGAGCGATTCTCCGGCGCCAATTCGG |
| | | GGAATACGGCCACCCTGACCATCAGCGGGGCCCTGGCCG |
| | | AGGATGAGGCTGACTATTACTGTCAGGTGTGGGACAGTAG |
| | | TACTAGTGTGTTCGGCGGAGGCACCCATCTGACCGTCCTC |
| | | G |
| 444 | PMX084 light chain | SYVLTQLPSVSVTLRQTARITCGGDSIGSKNVYWHQQKLGQA |
| | variable region amino | PVLIIYDDSSRPSGIPERFSGANSGNTATLTISGALAEDEADYY |
| | acid sequence | CQVWDSSTSVFGGGTHLTVL |
| 445 | PMX084 heavy chain | GFTFSNY |
| | CDR1 | |
| 446 | PMX084 heavy chain | SNDGRI |
| | CDR2 | |
| 447 | PMX084 heavy chain | CAKETSRWGFDYW |
| | CDR3 | |
| 448 | PMX084 light chain | GGDSIGSKNVY |
| | CDR1 | |
| 449 | PMX084 light chain | DDSSRPS |
| | CDR2 | |
| 450 | PMX084 light chain | CQVWDSSTSVF |
| | CDR3 | |
| 451 | PMX085 Heavy chain | GAGGTTAAGCTGGTGGAGTCTGGGGGAGACCTTGTGAAAC |
| | variable region nucleic | CTGAGGGGTCCCTGAGACTCTCCTGTGTGGTCTCTGGCTT |
| | acid sequence | CACCTTTAGTAGGTACGGCATGAGCTGGGTCCGCCAGGCT |
| | | CCAGGGAAGGGGCTGCAGTGGGTCGCGTACATTAGCAGT |
| | | GATGGAAGGAGCTCAAGTTACACAGACGCTGTGCAGGGCC |
| | | GATTCACCATCTCCAGCGACAATGCCAAGAACACGCTGTAT |
| | | CTGCAGATGAACAGCCTGACAACTGAGGACACAGCCGTGT |
| | | ATTACTGTGCGAAGTTGGGGCGCGGATTTAATTTTAACTTC |
| | | TGGGGCCAGGGAACCCTGGTCACCGTCTCCTCAG |
| 452 | PMX085 Heavy chain | EVKLVESGGDLVKPEGSLRLSCVVSGFTFSRYGMSWVRQAP |
| | variable region amino | GKGLQWVAYISSDGRSSSYTDAVQGRFTISSDNAKNTLYLQM |
| | acid sequence | NSLTTEDTAVYYCAKLGRGFNFNFWGQGTLVTVSS |
| 453 | PMX085 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTACTGATTATCTATTATGATAGCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAGTGTGTTCGGCGGAGGCACCCATCTGACCGTCCTCG |
| 454 | PMX085 light chain | SYVLTQLPSKNVTLKQPAHITCGGDNIGSKSVHWYQQKLGQA |
| | variable region amino | PVLIIYYDSSRPTGIPERFSGANSGNTATLTISGALAEDEADYY |
| | acid sequence | CQVWDSSASVFGGGTHLTVL |
| 455 | PMX085 heavy chain | GFTFSRY |
| | CDR1 | |
| 456 | PMX085 heavy chain | SSDGRS |
| | CDR2 | |
| 457 | PMX085 heavy chain | CAKLGRGFNFNFW |
| | CDR3 | |
| 458 | PMX085 light chain | GGDNIGSKSVH |
| | CDR1 | |
| 459 | PMX085 light chain | YDSSRPT |
| | CDR2 | |
| 460 | PMX085 light chain | CQVWDSSASVF |
| | CDR3 | |
| 461 | PMX086 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAGCTACCACATGAGCTGGGTCCGCCAGGC |
| | | TCCAGGGAAGGGACTTCAGTGGGTCGCATTCATTAACAGT |
| | | GCTGAAAGTAGCACAACCTATGCAGACGCTGTGAAGGGCC |
| | | GATTCACCATCTCCAGAGACAACGCCAAGAACACGCTGTA |
| | | TCTTCAGATGAACAGCCTGAGAGCCGAGGACACGGCCGTA |
| | | TATTACTGTGCGGGTGGATATATATGGACCCATTTTGACTA |
| | | CTGGGGCCAGGGAACCCTGGTCACCGTCTCCTCAG |
| 462 | PMX086 Heavy chain | EVQLVESGGDLVKPGGSLRLSCVASGFTFSSYHMSWVRQAP |
| | variable region amino | GKGLQWVAFINSAESSTTYADAVKGRFTISRDNAKNTLYLQM |
| | acid sequence | NSLRAEDTAVYYCAGGYIWTHFDYWGQGTLVTVSS |
| 463 | PMX086 light chain | TCCTATGTGCTGACACAGCTGCCATCCGTGAGTGTGACCC |
| | variable region | TGAGGCAGACGGCCCGCATCACCTGTGGGGGAGACATCA |
| | nucleotide sequence | TTGGAAGTAAAAGTGTTTACTGGTACCAGCAGAATCTGGGC |
| | | CAGGCCCCTGTACTGATTATCTATAGAGATACCAACAGGCC |
| | | GACAGGGATCCCTGAGCGATTCTCTGGCGCCAGCTCGGG |
| | | GAACACGGCCACCCTGACCATCAGCGGGGCCCTGGCCGA |
| | | GGACGAGGCTGACTATTACTGCCAGGTGTGGGACAGCAGT |
| | | ACTAGTGTGTTCGGCGGAGGCACCCATCTGACCGTCCTCG |
| 464 | PMX086 light chain | SYVLTQLPSVSVTLRQTARITCGGDIIGSKSVYWYQQNLGQAP |
| | variable region amino | VLIIYRDTNRPTGIPERFSGASSGNTATLTISGALAEDEADYYC |
| | acid sequence | QVWDSSTSVFGGGTHLTVL |
| 465 | PMX086 heavy chain | GFTFSSY |
| | CDR1 | |
| 466 | PMX086 heavy chain | NSAESS |
| | CDR2 | |
| 467 | PMX086 heavy chain | CAGGYIWTHFDYW |
| | CDR3 | |
| 468 | PMX086 light chain | GGDIIGSKSVY |
| | CDR1 | |
| 469 | PMX086 light chain | RDTNRPT |
| | CDR2 | |
| 470 | PMX086 light chain | CQVWDSSTSVF |
| | CDR3 | |
| 471 | PMX087 Heavy chain | GAGGTGCACCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAACTACCACATGAGCTGGGTCCGCCAGGC |
| | | TCCAGGGAAGGGGCTTCAGTGGGTCGCATTCATTAACAGT |
| | | GGTGGAAGTGGCACAAGCTATGCAGACGCTGTGAAGGGC |
| | | CGATTCACCATCTCCAGAGACTACGCCAAGAGCACGCTAT |
| | | ATCTTCAGATGAACAGCCTGAGAGCCGAGGACACGGCCGT |
| | | CTATTACTGTGCGAGTGGATATATATGGACCCATTTTGACT |
| | | ACTGGGGCCAGGGAACCCTGGTCACCGTCTCCTCAG |
| 472 | PMX087 Heavy chain | EVHLVESGGDLVKPGGSLRLSCVASGFTFSNYHMSWVRQAP |
| | variable region amino | GKGLQWVAFINSGGSGTSYADAVKGRFTISRDYAKSTLYLQM |
| | acid sequence | NSLRAEDTAVYYCASGYIWTHFDYWGQGTLVTVSS |
| 473 | PMX087 light chain | TCCTATGTGCTGACACAGCTGCCATCCGTGAGTGTGACCC |
| | variable region | TGAGGCAGACGGCCCGCATCACCTGTGGGGGAGACATCA |
| | nucleotide sequence | TTGGAAGTAAAAATGTTTACTGGTACCAGCAGAAGCTGGG |
| | | CCAGGCCCCTATACTGATTATCTATAGAGATAGCAACAGGC |
| | | CGACAGGGATCCCTGAGCGATTCTCTGGCGCCAGCTCGG |
| | | GGAACACGGCCACCCTGACCATCAGCGGGGCCCTGGCCG |
| | | AGGACGAGGCTGACTATTACTGCCAGGTGTGGGACAGCAG |
| | | TACTAGTGTGTTCGGCGGAGGCACCCATCTGACCGTCCTC |
| | | G |
| 474 | PMX087 light chain | SYVLTQLPSVSVTLRQTARITCGGDIIGSKNVYWYQQKLGQAP |
| | variable region amino | ILIIYRDSNRPTGIPERFSGASSGNTATLTISGALAEDEADYYC |
| | acid sequence | QVWDSSTSVFGGGTHLTVL |
| 475 | PMX087 heavy chain | GFTFSNY |
| | CDR1 | |
| 476 | PMX087 heavy chain | NSGGSG |
| | CDR2 | |
| 477 | PMX087 heavy chain | CASGYIWTHFDYW |
| | CDR3 | |
| 478 | PMX087 light chain | GGDIIGSKNVY |
| | CDR1 | |
| 479 | PMX087 light chain | RDSNRPT |
| | CDR2 | |
| 480 | PMX087 light chain | CQVWDSSTSVF |
| | CDR3 | |
| 481 | PMX088 Heavy chain | GAGGTGCAGTTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAATACCTACCACATGAACTGGGTCCGCCAGGC |
| | | TCCAGGGAAGGGGCTTCACTGGGTCGCATTCATTAACAGC |
| | | GGTGGGACTGGCACAGGCTATTCAGACACTGTGAAGGGC |
| | | CGATTCACCATCTCCAGAGACTACGCCAAGAATACACTCTA |
| | | TCTTCAGATGAATAGTCTGAGAGCCGAAGACACGGCCGTT |
| | | TATTATTGTGTGAGTGGATCTATATGGACTCATTTTGACTAC |
| | | TGGGGCCAGGGAACCCTGGTCACCGTCTCCTCAG |
| 482 | PMX088 Heavy chain | EVQLVESGGDLVKPGGSLRLSCVASGFTFNTYHMNWVRQAP |
| | variable region amino | GKGLHWVAFINSGGTGTGYSDTVKGRFTISRDYAKNTLYLQM |
| | acid sequence | NSLRAEDTAVYYCVSGSIWTHFDYWGQGTLVTVSS |
| 483 | PMX088 light chain | TCCTATGTGCTGACACAGCTGCCATCCGTGAGTATGACCC |
| | variable region | TGAGGCAGACGGCCCACATCACCTGTGGGGGAGACATCAT |
| | nucleotide sequence | TGGAAGTAAAAGTGTTTACTGGTACCAGCAGAAGCTGGGC |
| | | CAGGCCCCTGTACTGATTATTTATAGAGATACCAACAGGCC |
| | | GACAGGGATCCCTGAGCGATTCTCTGGCGCCAGCTCGGG |
| | | GAACACGGCCACCCTGACCATCAGCGGGGCCCTGGCCGA |
| | | GGACGAGGCTGACTATTACTGCCAGGTTTGGGACAGCAAT |
| | | ACTCAGGCTGTGTTCGGCGGAGGCACCCATCTGACCGTCC |
| | | TCG |
| 484 | PMX088 light chain | SYVLTQLPSVSMTLRQTAHITCGGDIIGSKSVYWYQQKLGQA |
| | variable region amino | PVLIIYRDTNRPTGIPERFSGASSGNTATLTISGALAEDEADYY |
| | acid sequence | CQVWDSNTQAVFGGGTHLTVL |
| 485 | PMX088 heavy chain | GFTFNTY |
| | CDR1 | |
| 486 | PMX088 heavy chain | NSGGTG |
| | CDR2 | |
| 487 | PMX088 heavy chain | CVSGSIWTHFDYW |
| | CDR3 | |
| 488 | PMX088 light chain | GGDIIGSKSVY |
| | CDR1 | |
| 489 | PMX088 light chain | RDTNRPT |
| | CDR2 | |
| 490 | PMX088 light chain | CQVWDSNTQAVF |
| | CDR3 | |
| 491 | PMX089 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAATAACTTCCACATGAGTTGGGTCCGCCAGGCT |
| | | CCAGGGAAGGGGCTTCAGTGGGTCGCATTCATTAACAGTG |
| | | ATGGAGGTAATAGAAACTATGCAGACGTTGTGAAGGGCCG |
| | | AGTCACCATCTCCAGAGACTACGCCAAGAATACGCTGTATC |
| | | TTCAGATAAACAGTCTGAGAGCCGAGGACACGGCCGTGTA |
| | | TTACTGTGCGGGTGGATCTATATGGACTCACTTTGACTACT |
| | | GGGGCCAGGGAACCCTGGTCACCGTCTCCTCAG |
| 492 | PMX089 Heavy chain | EVQLVESGGDLVKPGGSLRLSCVASGFTFNNFHMSWVRQAP |
| | variable region amino | GKGLQWVAFINSDGGNRNYADVVKGRVTISRDYAKNTLYLQI |
| | acid sequence | NSLRAEDTAVYYCAGGSIWTHFDYWGQGTLVTVSS |
| 493 | PMX089 light chain | TCCTATGTGTTGACACAGCTGCCATCCATGAGTGTGACCCT |
| | variable region | GAGGCAGACGGCCCGCATCACCTGTGAGGGAGACATCATT |
| | nucleotide sequence | GCAAGTAAAAGAATTTACTGGTACCAACAGAAGCTGGGCC |
| | | AGGTCCCTGTACTGCTTATCTATGCTGATAGCAGCAGGCC |
| | | GTCAGGGATCCCTGAACGATTCTCCGGCGCCAGCTCGGG |
| | | GAACACAGCCACCCTGACCATCAGCGGGGCCCTGGCCGA |
| | | GGACGAGGCTGACTATTACTGCCAGGTGTGGGACAACAAT |
| | | GCTAATAGTGTGTTCGGCGGAGGCACCCATCTGACCGTCC |
| | | TCG |
| 494 | PMX089 light chain | SYVLTQLPSMSVTLRQTARITCEGDIIASKRIYWYQQKLGQVP |
| | variable region amino | VLLIYADSSRPSGIPERFSGASSGNTATLTISGALAEDEADYYC |
| | acid sequence | QVWDNNANSVFGGGTHLTVL |
| 495 | PMX089 heavy chain | GFTFNNF |
| | CDR1 | |
| 496 | PMX089 heavy chain | NSDGGN |
| | CDR2 | |
| 497 | PMX089 heavy chain | CAGGSIWTHFDYW |
| | CDR3 | |
| 498 | PMX089 light chain | EGDIIASKRIY |
| | CDR1 | |
| 499 | PMX089 light chain | ADSSRPS |
| | CDR2 | |
| 500 | PMX089 light chain | CQVWDNNANSVF |
| | CDR3 | |
| 501 | PMX090 Heavy chain | GAGGTACAGTTGGTGGAATCTGGGGGAGACCTGGTGAAG |
| | variable region nucleic | CCTGGGGGGTCCCTGAGACTTTCCTGTGTGGCCTCTGGAT |
| | acid sequence | TCACCTTCAATACCTACCACATGAACTGGGTCCGCCAGGC |
| | | TCCAGGGAAGGGACTTCACTGGGTCGCATTCATTAACAGC |
| | | GGTGGAGGTGGCACAAGCTATGCAGACACTGTGCAGGGC |
| | | CGATTCACCATCTCCAGAGACTACGCCAAGAACGCACTCT |
| | | ATCTTCAGATGAACAGCCTGAGAGCCGAGGACACGGCCGT |
| | | TTATTACTGTGCGAGTGGATCTATATGGACTCATTTTGACTA |
| | | CTGGGGCCAGGGAACCCTGGTCACCGTCTCCTCAG |
| 502 | PMX090 Heavy chain | EVQLVESGGDLVKPGGSLRLSCVASGFTFNTYHMNWVRQAP |
| | variable region amino | GKGLHWVAFINSGGGGTSYADTVQGRFTISRDYAKNALYLQ |
| | acid sequence | MNSLRAEDTAVYYCASGSIWTHFDYWGQGTLVTVSS |
| 503 | PMX090 light chain | TCCTATGTGCTGACACAGCTGCCATTCATGCGTGTGACCCT |
| | variable region | GAGTCAGACGGCCCGCATCACCTGTGAGGGAGACAACATT |
| | nucleotide sequence | GGAAGTAAAAGAGTTTTCTGGTACCAGCAGAAGCCGGGCC |
| | | AGGTCCCTGTAGTGATTATTCATGATGATAGCAGCAGGCC |
| | | GTCAGGGATCCCTGAGCGATTCTCCGGCGCCAATTCGGG |
| | | GAACACAGCCACCCTGACCATCAGCGGGGCCCTGGCCGA |
| | | GGACGAGGCTGCCTATTACTGCCAGGTGTGGGACAGCAGT |
| | | ACTAATAGTGTGTTCGGCGGGGGCACCCATCTGACCGTCC |
| | | TCG |
| 504 | PMX090 light chain | SYVLTQLPFMRVTLSQTARITCEGDNIGSKRVFWYQQKPGQV |
| | variable region amino | PVVIIHDDSSRPSGIPERFSGANSGNTATLTISGALAEDEAAYY |
| | acid sequence | CQVWDSSTNSVFGGGTHLTVL |
| 505 | PMX090 heavy chain | GFTFNTY |
| | CDR1 | |
| 506 | PMX090 heavy chain | NSGGGG |
| | CDR2 | |
| 507 | PMX090 heavy chain | CASGSIWTHFDYW |
| | CDR3 | |
| 508 | PMX090 light chain | EGDNIGSKRVF |
| | CDR1 | |
| 509 | PMX090 light chain | DDSSRPS |
| | CDR2 | |
| 510 | PMX090 light chain | CQVWDSSTNSVF |
| | CDR3 | |
| 511 | PMX091 Heavy chain | GAGGTGCAGCTGGTGGAGTCTGGGGGAGACCTGGTGAGG |
| | variable region nucleic | CCTGGGGGGTCCCTGAGACTTTCCTGTGTGGCCTCTGGAT |
| | acid sequence | TCACCTTCAGTAACTACCACATGAATTGGGTCCGCCAGGCT |
| | | CCAGGGAAGGGGCTTCAGTGGGTCGCATTCATTAACAGTG |
| | | GTGGAGGTGGCACATCCTATGCAGACGTTGTGAGGGGCC |
| | | GATTCACCATCTCCAGAGACTACGCCAAAAACACATTGTAT |
| | | CTTCAGATGAACAGCCTGAGCGCCGAGGACACGGCCGTAT |
| | | ATTACTGTGCGAGTGGATCTATATGGACTCATTTTGACTAC |
| | | TGGGGCCAGGGAACCCTGGTCACCGTCTCCTCAG |
| 512 | PMX091 Heavy chain | EVQLVESGGDLVRPGGSLRLSCVASGFTFSNYHMNWVRQAP |
| | variable region amino | GKGLQWVAFINSGGGGTSYADVVRGRFTISRDYAKNTLYLQ |
| | acid sequence | MNSLSAEDTAVYYCASGSIWTHFDYWGQGTLVTVSS |
| 513 | PMX091 light chain | TCCTATGTGCTGACACAGCTGACATCCATGAGTGTGACCCT |
| | variable region | GAGGCAGGCGGCCCGCTTCACCTGTGAGAGAGACACCAT |
| | nucleotide sequence | TGGAAGTAAAAGAGTTTACTGGTACCAGCAGAGGCTGGGC |
| | | CAGGTCCCTGTCCTGATTATCTATGATTATAACATCAGGCC |
| | | GTCAGGGATCCCTGAGCGACTCTCCGGCGCCTATTCGGG |
| | | GAACACAGCCGCCCTGACCATCAGCGGGGCCCTGGCCGA |
| | | GGACGAGGCTGACTATTTCTGCCAGGTGTGGGACAGCAGT |
| | | GCTAATAGTGTGTTCGGCGGAGGCACCCATCTGACCGTCC |
| | | TCG |
| 514 | PMX091 light chain | SYVLTQLTSMSVTLRQAARFTCERDTIGSKRVYWYQQRLGQ |
| | variable region amino | VPVLIIYDYNIRPSGIPERLSGAYSGNTAALTISGALAEDEADYF |
| | acid sequence | CQVWDSSANSVFGGGTHLTVL |
| 515 | PMX091 heavy chain | GFTFSNY |
| | CDR1 | |
| 516 | PMX091 heavy chain | NSGGGG |
| | CDR2 | |
| 517 | PMX091 heavy chain | CASGSIWTHFDYW |
| | CDR3 | |
| 518 | PMX091 light chain | ERDTIGSKRVY |
| | CDR1 | |
| 519 | PMX091 light chain | DYNIRPS |
| | CDR2 | |
| 520 | PMX091 light chain | CQVWDSSANSVF |
| | CDR3 | |
| 521 | PMX092 Heavy chain | GAGGTGCAGTTGGTGGAGTCTGGGGGAGACCTGGTGAAG |
| | variable region nucleic | CCGGGGGGGTCCCTGAGACTTTCCTGTGTGGCCTCTGGAT |
| | acid sequence | TCACCTTCAATAACCTCCACATGAGTTGGGTCCGCCAGGC |
| | | TCCAGGGAAGGGGCTTCAGTGGGTCGCATTCATTAACAGT |
| | | GATGGACGTAATAGAAACTATGCAGACGCTGTGAAGGGCC |
| | | GAGTCACCATCTCCAGAGACTACGCCAAGAATACGCTGTA |
| | | TCTTCAGATAAACAGTCTGAGAGCCGAAGACACGGCCGTG |
| | | TATTACTGTACGGGTGGATCTATATGGACTCACTTTGACTA |
| | | CTGGGGCCAGGGAACCCTGGTCACCGTCTCCTCAG |
| 522 | PMX092 Heavy chain | EVQLVESGGDLVKPGGSLRLSCVASGFTFNNLHMSWVRQAP |
| | variable region amino | GKGLQWVAFINSDGRNRNYADAVKGRVTISRDYAKNTLYLQI |
| | acid sequence | NSLRAEDTAVYYCTGGSIWTHFDYWGQGTLVTVSS |
| 523 | PMX092 light chain | TCCTATGTGTTGACACAGCTGCCATCCATGAGTGTGACCCT |
| | variable region | GAGGCAGACGGCCCGCATCACCTGTGAGGGAGACATCATT |
| | nucleotide sequence | GCAAGTAAAAGAGTTTACTGGTACCAACAGAAGCTGGGCC |
| | | AGGTCCCTGTACTGTTTATCTATGCTGATAACAGCAGGCCG |
| | | TCAGGGATCCCTGAACGATTCTCCGGCGCCAGCTCGGGG |
| | | AACACAGCCACCCTGACCATCAGCGGGGCCCTGGCCGAG |
| | | GACGAGGCTGACTATTACTGCCAGGTGTGGGACAGCAATG |
| | | CTAATAGTGTGTTCGGCGGAGGCACCCATCTGACCGTCCT |
| | | CG |
| 524 | PMX092 light chain | SYVLTQLPSMSVTLRQTARITCEGDIIASKRVYWYQQKLGQVP |
| | variable region amino | VLFIYADNSRPSGIPERFSGASSGNTATLTISGALAEDEADYY |
| | acid sequence | CQVWDSNANSVFGGGTHLTVL |
| 525 | PMX092 heavy chain | GFTFNNL |
| | CDR1 | |
| 526 | PMX092 heavy chain | NSDGRN |
| | CDR2 | |
| 527 | PMX092 heavy chain | CTGGSIWTHFDYW |
| | CDR3 | |
| 528 | PMX092 light chain | EGDIIASKRVY |
| | CDR1 | |
| 529 | PMX092 light chain | ADNSRPS |
| | CDR2 | |
| 530 | PMX092 light chain | CQVWDSNANSVF |
| | CDR3 | |
| 531 | PMX093 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAGCTACCACATGAGCTGGGTCCGCCAGGC |
| | | TCCAGGGAAGGGGCTTCAGTGGGTCGCATTCATTAACAGT |
| | | GGTGGAGGTGGCACAAACTATGCAGACGCTGTGAAGGGC |
| | | CGATTCACCATCTCCAGAGACTACGCCAAGAATACGCTGTA |
| | | TCTTCAGATGAACAGCCTGAGAGCCGAGGACACGGCCGTG |
| | | TATTACTGTGTGGGTGGATCTATATGGACTCATTTTGACTA |
| | | CTGGGGCCAGGGAACCCTGGTCACCGTCTCCTCAG |
| 532 | PMX093 Heavy chain | EVQLVESGGDLVKPGGSLRLSCVASGFTFSSYHMSWVRQAP |
| | variable region amino | GKGLQWVAFINSGGGGTNYADAVKGRFTISRDYAKNTLYLQ |
| | acid sequence | MNSLRAEDTAVYYCVGGSIWTHFDYWGQGTLVTVSS |
| 533 | PMX093 light chain | TCCTATGTGCTGACACAGCTGCCATCCATGAGTGTGACCC |
| | variable region | TGAGGCAGACGGCCCGCATCACCTGTGAGGGAGACATCAT |
| | nucleotide sequence | TGCAAGTAAAAGAGTTTACTGGTACCAACAGAAGCTGGGC |
| | | CAGGTCCCTGTACTGCTTATCTATGGTGATAGCAGCAGGC |
| | | CGTCAGGGATCCCTGAGCGATTCTCCGGCGCCAACTCGG |
| | | GGAACACAGCCACCCTGACCATCAGCGGGGCCCTGGCCG |
| | | AGGACGAGGCTGACTATTACTGCCAGGTGTGGGACAGCG |
| | | GTACTAATAGTGTGTTCGGCGGAGGCACCCATCTGACCGT |
| | | CCTCG |
| 534 | PMX093 light chain | SYVLTQLPSMSVTLRQTARITCEGDIIASKRVYWYQQKLGQVP |
| | variable region amino | VLLIYGDSSRPSGIPERFSGANSGNTATLTISGALAEDEADYY |
| | acid sequence | CQVWDSGTNSVFGGGTHLTVL |
| 535 | PMX093 heavy chain | GFTFSSY |
| | CDR1 | |
| 536 | PMX093 heavy chain | NSGGGG |
| | CDR2 | |
| 547 | PMX093 heavy chain | CVGGSIWTHFDYW |
| | CDR3 | |
| 538 | PMX093 light chain | EGDIIASKRVY |
| | CDR1 | |
| 539 | PMX093 light chain | GDSSRPS |
| | CDR2 | |
| 540 | PMX093 light chain | CQVWDSGTNSVF |
| | CDR3 | |
| 541 | PMX094 Heavy chain | GACATACATCTGGTGGAGTCTGGGGGAGACCTGGTGAGG |
| | variable region nucleic | CCTGGGGGGTCCCTGAGACTTTCCTGTGTGGCCTCTGGAT |
| | acid sequence | TCACCTTCAGTAACCTCCACATGAACTGGGTCCGCCAGGC |
| | | TCCAGGGAAGGGGCTTCAGTGGGTCGCATTCATTAACAGT |
| | | GGTGGAAGTGGCACAACCTATGCAGACGTTGTGAGGGGC |
| | | CGATTCACCATCTCCAGAGACTACGCCAAAAACACACTGTA |
| | | TCTTCAGATGAATAGCCTGAGAGCCGACGACACGGCCGTA |
| | | TATTACTGTGCGAGTGGATCTATATGGACTCATTTTGACTA |
| | | CTGGGGCCAGGGAACCCTGGTCACCGTCTCCTCAG |
| 542 | PMX094 Heavy chain | DIHLVESGGDLVRPGGSLRLSCVASGFTFSNLHMNWVRQAP |
| | variable region amino | GKGLQWVAFINSGGSGTTYADVVRGRFTISRDYAKNTLYLQM |
| | acid sequence | NSLRADDTAVYYCASGSIWTHFDYWGQGTLVTVSS |
| 543 | PMX094 light chain | TCCTTTGTGTTGACACAGCTGCCATCCGTGAGTGTGACCCT |
| | variable region | GAGGCAGACGGCCCGCATCACCTGTGGGGGAGACAGTAT |
| | nucleotide sequence | TGGAAGTAAAAGTGTTTACTGGTACCAGCAGAAGTTGGGC |
| | | CAGGCCCCTGTACTGATTATCTATAGAGATAGCAACAGGC |
| | | CGACAGGGATCCCTGAGCGAATCTCTGGCGCCAGCTCGG |
| | | GGAACACGGCCACCCTGACCATCAGCGGGGCCCTGGCCG |
| | | AGGACGAGGCTGACTATTACTGCCAGGTGTGGGACAGCAG |
| | | TAATAAGGCTATGTTCGGCGGAGGCACCCATCTGACCGTC |
| | | CTCG |
| 544 | PMX094 light chain | SFVLTQLPSVSVTLRQTARITCGGDSIGSKSVYWYQQKLGQA |
| | variable region amino | PVLIIYRDSNRPTGIPERISGASSGNTATLTISGALAEDEADYY |
| | acid sequence | CQVWDSSNKAMFGGGTHLTVL |
| 545 | PMX094 heavy chain | GFTFSNL |
| | CDR1 | |
| 546 | PMX094 heavy chain | NSGGSG |
| | CDR2 | |
| 547 | PMX094 heavy chain | CASGSIWTHFDYW |
| | CDR3 | |
| 548 | PMX094 light chain | GGDSIGSKSVY |
| | CDR1 | |
| 549 | PMX094 light chain | RDSNRPT |
| | CDR2 | |
| 550 | PMX094 light chain | CQVWDSSNKAMF |
| | CDR3 | |
| 551 | PMX095 Heavy chain | GCGGTGCAGCTGGTGGAGTCTGGGGGAGACCTGGTGAGG |
| | variable region nucleic | CCTGGGGGGTCCCTGAGACTTTCCTGTGTGGCCTCTGGAT |
| | acid sequence | TCACCTTCAGTAGTTACCACATGAACTGGGTCCGCCAGGC |
| | | TCCTGGGAAGGGGCTTCAGTGGGTCGCATTCATTAACAGT |
| | | GGAGGAAGTAGTACAACCTATGCAGACGTTGTCAGGGGCC |
| | | GATTCACCATCTCCAGAGACTACGCCAAAAACACGCTATTT |
| | | CTTCAGATGAACAGCCTGAGAGCCGAGGACACGGCCGTGT |
| | | ATTATTGTGCGATTGGATCTATATGGACACATTTTGACTACT |
| | | GGGGCCAGGGAACCCTGGTCACCGTCTCCTCAG |
| 552 | PMX095 Heavy chain | AVQLVESGGDLVRPGGSLRLSCVASGFTFSSYHMNWVRQAP |
| | variable region amino | GKGLQWVAFINSGGSSTTYADVVRGRFTISRDYAKNTLFLQM |
| | acid sequence | NSLRAEDTAVYYCAIGSIWTHFDYWGQGTLVTVSS |
| 553 | PMX095 light chain | TCCTTTGTGCTGACACAGCTGCCATCCGTGAGTGTGACCC |
| | variable region | TGAGGCAGACGGCCCGCATCACCTGTGGGGGAGACATCA |
| | nucleotide sequence | TTGGAAGTAAAAATGTTTACTGGTACCAGCAGAGGCTGGG |
| | | CCAGGCCCCTGTATTGATTCTCTATAGAGATACCAACAGGC |
| | | CGACAGGGATCCCTGAGCGATTCTCTGGCGCCAGCTCGG |
| | | GGAACACGGCCACCCTGACCATCAGCGGGGCCCTGGCCG |
| | | AGGACGAGGCTGACTATTACTGCCAGGTGTGGGACAACAG |
| | | TAATAAGGCTATGTTCGGCGGAGGCACCCATCTGACCGTC |
| | | CTCG |
| 554 | PMX095 light chain | SFVLTQLPSVSVTLRQTARITCGGDIIGSKNVYWYQQRLGQAP |
| | variable region amino | VLILYRDTNRPTGIPERFSGASSGNTATLTISGALAEDEADYYC |
| | acid sequence | QVWDNSNKAMFGGGTHLTVL |
| 555 | PMX095 heavy chain | GFTFSSY |
| | CDR1 | |
| 556 | PMX095 heavy chain | NSGGSS |
| | CDR2 | |
| 557 | PMX095 heavy chain | CAIGSIWTHFDYW |
| | CDR3 | |
| 558 | PMX095 light chain | GGDIIGSKNVY |
| | CDR1 | |
| 559 | PMX095 light chain | RDTNRPT |
| | CDR2 | |
| 560 | PMX095 light chain | CQVWDNSNKAMF |
| | CDR3 | |
| 561 | PMX096 Heavy chain | GAGGTACAGTTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAATACTTACCACATGAACTGGGTCCGCCAGGCT |
| | | CCAGGGAAGGGGCTTCACTGGGTCGCATTTATTAACAGCG |
| | | GTGGAAGTGACACAAGCTATGCAGACACTGTGCAGGGCCG |
| | | GTTCACCATCTCCAGAGACTACGCCAAGAACACACTCTATC |
| | | TTCAGATGAACAGCCTGAGAGCCGAGGACACGGCCGTGTA |
| | | TTACTGTGCGAGTGGATATATATGGACTCATTTTGACTACT |
| | | GGGGCCAGGGAACCCTAGTCACCGTCTCCTCAG |
| 562 | PMX096 Heavy chain | EVQLVESGGDLVKPGGSLRLSCVASGFTFNTYHMNWVRQAP |
| | variable region amino | GKGLHWVAFINSGGSDTSYADTVQGRFTISRDYAKNTLYLQM |
| | acid sequence | NSLRAEDTAVYYCASGYIWTHFDYWGQGTLVTVSS |
| 563 | PMX096 light chain | TCCTATGTGCTGACACAGCTGCCATCCGTGAGTGTGACCC |
| | variable region | TGAGGCAGACGGCCCACATCACCTGTGGGGGAGACATCAT |
| | nucleotide sequence | TGGAAGTAAAAGTGTTTACTGGTACCAGCAGAAGCTGGGC |
| | | CAGGCCCCTGTAATGATTATCTATAGAGATAACAACAGGCC |
| | | GACAGGGATCCCTGAGCGATTCTCTGGCGCCAGCTCGGG |
| | | GAACACGGCCACCCTGACCATCAGCGGGTCCCTGGCCGA |
| | | GGACGAGGCTGACTATTACTGCCAGGTTTGGGACAGCAGG |
| | | ACTCAGGCTTTGTTCGGCGGAGGCACCCATCTGACCGTCC |
| | | TCG |
| 564 | PMX096 light chain | SYVLTQLPSVSVTLRQTAHITCGGDIIGSKSVYWYQQKLGQAP |
| | variable region amino | VMIIYRDNNRPTGIPERFSGASSGNTATLTISGSLAEDEADYY |
| | acid sequence | CQVWDSRTQALFGGGTHLTVL |
| 565 | PMX096 heavy chain | GFTFNTY |
| | CDR1 | |
| 566 | PMX096 heavy chain | NSGGSD |
| | CDR2 | |
| 567 | PMX096 heavy chain | CASGYIWTHFDYW |
| | CDR3 | |
| 568 | PMX096 light chain | GGDIIGSKSVY |
| | CDR1 | |
| 569 | PMX096 light chain | RDNNRPT |
| | CDR2 | |
| 570 | PMX096 light chain | CQVWDSRTQALF |
| | CDR3 | |
| 571 | PMX097 Heavy chain | GAGGTGCAGCTGGTGGAGTCTGGGGGAGACCTGGTGAGG |
| | variable region nucleic | CCTGGGGGGTCCCTGAGACTTTCCTGTGTGGCCTCTGGAA |
| | acid sequence | TCACCTTCAGTAGTTACCACATGAGTTGGGTCCGCCAGGC |
| | | TCCTGGGAAGGGGCTTCAGTGGGTCGCACTCATAAACAGT |
| | | GGTGGAAGTAGTATAACCTATGCAGACGTTGTGAGGGGCC |
| | | GATTCACCATCTCCAGAGACTACGCCAAAAACACGCTGTAT |
| | | CTTCAGATGAACAGCCTGAGAGCCGAGGACACGGCCGTGT |
| | | ATTATTGTGCGAGTGGATCTATATGGACTCATTTTGACTATT |
| | | GGGGCCAGGGGACCCGGGTCACCGTCTCCTCAG |
| 572 | PMX097 Heavy chain | EVQLVESGGDLVRPGGSLRLSCVASGITFSSYHMSWVRQAP |
| | variable region amino | GKGLQWVALINSGGSSITYADVVRGRFTISRDYAKNTLYLQM |
| | acid sequence | NSLRAEDTAVYYCASGSIWTHFDYWGQGTRVTVSS |
| 573 | PMX097 light chain | TCCTTTGTGCTGACACAGCTGCCATCCGTGAGTGTGACCC |
| | variable region | TGAGGCAGACGGCCCGCATCACCTGTGGGGGAGACATCA |
| | nucleotide sequence | TTGGAAGTAAAAATGTTTACTGGTATCAACAGAGGCTGGGC |
| | | CAGGCCCCTGTATTGATTATCTATAGAGATAGCAACAGGCC |
| | | GACAGGGATCCCTGAGCGATTCTCTGGCGCCAGCTCGGG |
| | | GAACACGGCCACCCTGACCATCAGCGGGGCCCTGGCCGA |
| | | GGACGAGGCTGACTATTACTGCCAGGTGTGGGACAACAGT |
| | | AATAAGGCTATGTTCGGCGGAGGCACCCATCTGACCGTCC |
| | | TCG |
| 574 | PMX097 light chain | SFVLTQLPSVSVTLRQTARITCGGDIIGSKNVYWYQQRLGQAP |
| | variable region amino | VLIIYRDSNRPTGIPERFSGASSGNTATLTISGALAEDEADYYC |
| | acid sequence | QVWDNSNKAMFGGGTHLTVL |
| 575 | PMX097 heavy chain | GITFSSY |
| | CDR1 | |
| 576 | PMX097 heavy chain | NSGGSS |
| | CDR2 | |
| 577 | PMX097 heavy chain | CASGSIWTHFDYW |
| | CDR3 | |
| 578 | PMX097 light chain | GGDIIGSKNVY |
| | CDR1 | |
| 579 | PMX097 light chain | RDSNRPT |
| | CDR2 | |
| 580 | PMX097 light chain | CQVWDNSNKAMF |
| | CDR3 | |
| 581 | PMX098 Heavy chain | GAGGTGCAATTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAATACCTACCACATGAACTGGGTCCGCCAGGC |
| | | TCCAGGGAAGGGGCTTCACTGGGTCGCATTCATTAACAGC |
| | | GGTGGAAGTGGCACAAGCTATGCAGACACTGTGAAGGGC |
| | | CGATTCACCATCTCCAGAGACTACGCCAAGAACACACTCTA |
| | | TCTTCAGATGAACAGCCTGAGAGCCGAGGACACGGCCGTG |
| | | TATTACTGTGCGAGTGGATCTATATGGACTCATTTTGACTA |
| | | CTGGGGCCAGGGAACCCTGGTCACCGTCTCCTCAG |
| 582 | PMX098 Heavy chain | EVQLVESGGDLVKPGGSLRLSCVASGFTFNTYHMNWVRQAP |
| | variable region amino | GKGLHWVAFINSGGSGTSYADTVKGRFTISRDYAKNTLYLQM |
| | acid sequence | NSLRAEDTAVYYCASGSIWTHFDYWGQGTLVTVSS |
| 583 | PMX098 light chain | TCCTATGTGCTGACACAGCTGCCATCCGTGAGTTTGACCCT |
| | variable region | GAGGCAGACGGCCCACATCACCTGTGGGGGAGACATCATT |
| | nucleotide sequence | GGAAGTAAAAGTGTTTACTGGTACCAGCAGAAGCTGGGCC |
| | | AGGCCCCTGTATTGATTATCTATAGAGATAACAACAGGCCG |
| | | ACAGGGATCCCTGAGCGATTCTCTGGCGCCAGCTCGGGG |
| | | AACACGGCCACCCTGACCATCAGCGGGGCCCTGGCCGAG |
| | | GACGAGGCTGACTATTACTGCCAGGTTTGGGACAGCAGTA |
| | | CTCAGGCTATGTTCGGCGGAGGCACCCATCTGACCGTCCT |
| | | CG |
| 584 | PMX098 light chain | SYVLTQLPSVSLTLRQTAHITCGGDIIGSKSVYWYQQKLGQAP |
| | variable region amino | VLIIYRDNNRPTGIPERFSGASSGNTATLTISGALAEDEADYYC |
| | acid sequence | QVWDSSTQAMFGGGTHLTVL |
| 585 | PMX098 heavy chain | GFTFNTY |
| | CDR1 | |
| 586 | PMX098 heavy chain | NSGGSG |
| | CDR2 | |
| 587 | PMX098 heavy chain | CASGSIWTHFDYW |
| | CDR3 | |
| 588 | PMX098 light chain | GGDIIGSKSVY |
| | CDR1 | |
| 589 | PMX098 light chain | RDNNRPT |
| | CDR2 | |
| 590 | PMX098 light chain | CQVWDSSTQAMF |
| | CDR3 | |
| 591 | PMX099 Heavy chain | GAGGTGCAGCTGGTGGAGTCTGGGGGAGACCTGGTGAGG |
| | variable region nucleic | CCTGGGGGGTCCCTGAGACTTTCCTGTGTGGCCTCTGGAT |
| | acid sequence | TCACCTTCAGTAGCTACCACATGAACTGGGTCCGCCAGGC |
| | | TCCAGGGAAGGGGCTTCAGTGGGTCGCATTCATTAACAGT |
| | | GGTGGAAGTAGCACAACCTATGCAGACGTTGTGAGGGGCC |
| | | GATTCACCATCTCCAGAGACTACGCCAAAAACACGCTGTAT |
| | | CTTCAGATGAACAGCCTGAGAGCCGAGGACACGGCCGTGT |
| | | ATTACTGTGCGAGTGGATCTATATGGACTCATTTTGACTAC |
| | | TGGGGCCAGGGAACCCTGGTCACCGTCTCCTCAG |
| 592 | PMX099 Heavy chain | EVQLVESGGDLVRPGGSLRLSCVASGFTFSSYHMNWVRQAP |
| | variable region amino | GKGLQWVAFINSGGSSTTYADVVRGRFTISRDYAKNTLYLQM |
| | acid sequence | NSLRAEDTAVYYCASGSIWTHFDYWGQGTLVTVSS |
| 593 | PMX099 light chain | TCCTTTGTGCTGACACAGCTGCCATCCGTAAGTGTGACCCT |
| | variable region | GAGGCAGACGGCCCGCATCACCTGTGGGGGAGACAGCAT |
| | nucleotide sequence | TGGAACTAAGAGTGTTTACTGGTACCAGCAGAAGCTGGGC |
| | | CAGGCCCCTGTATTGATTATCTATAGAGATAGCAACAGGCC |
| | | GACAGGGATCCCTGAGCGATTCTCTGGCGCCAGCTCGGG |
| | | GAACACGGCCACCCTGACCATCAGCGGGGCCCTGGCCGA |
| | | GGACGAGGCTGACTATTACTGCCAGGTGTGGGACAGCAGT |
| | | AATAAGGCTATGTTCGGCGGAGGCACCCATCTGACCGTCC |
| | | TCG |
| 594 | PMX099 light chain | SFVLTQLPSVSVTLRQTARITCGGDSIGTKSVYWYQQKLGQA |
| | variable region amino | PVLIIYRDSNRPTGIPERFSGASSGNTATLTISGALAEDEADYY |
| | acid sequence | CQVWDSSNKAMFGGGTHLTVL |
| 595 | PMX099 heavy chain | GFTFSSY |
| | CDR1 | |
| 596 | PMX099 heavy chain | NSGGSS |
| | CDR2 | |
| 597 | PMX099 heavy chain | CASGSIWTHFDYW |
| | CDR3 | |
| 598 | PMX099 light chain | GGDSIGTKSVY |
| | CDR1 | |
| 599 | PMX099 light chain | RDSNRPT |
| | CDR2 | |
| 600 | PMX099 light chain | CQVWDSSNKAMF |
| | CDR3 | |
| 601 | PMX100 Heavy chain | GAGGTGCAGCTGGTGGAGTCTGGGGGAGACCTGGTGAGG |
| | variable region nucleic | CCTGGGGGGTCCCTGAGACTTTCCTGTGTGGCCTCTGGAT |
| | acid sequence | TCACCTTCAGTAGTTACCACATGAGCTGGGTCCGCCAGGC |
| | | TCCTGGGAAGGGGCTTCAGTGGGTCGCATTCATTAACAGT |
| | | GGTGGAAGTAGTACAACCTATGCAGACGTTGTGAGGGGCC |
| | | GATTCACCATCTCCAGAGACTACGCCAAAAACACGCTGTAT |
| | | CTTCAGATGAACAGCCTGAGAGCCGAGGACACGGCCGTGT |
| | | ATTATTGTGCGAGTGGATCTATATGGACTCATTTTGACTACT |
| | | GGGGCCAGGGAACCCTGGTCACCGTCTCCTCAG |
| 602 | PMX100 Heavy chain | EVQLVESGGDLVRPGGSLRLSCVASGFTFSSYHMSWVRQAP |
| | variable region amino | GKGLQWVAFINSGGSSTTYADVVRGRFTISRDYAKNTLYLQM |
| | acid sequence | NSLRAEDTAVYYCASGSIWTHFDYWGQGTLVTVSS |
| 603 | PMX100 light chain | TCCTTTGTGCTGACACAGCTGCCATCCGTGAGTGTGACCC |
| | variable region | TGAGGCAGACGGCCCGCATCACCTGTGGGGGAGACAGCA |
| | nucleotide sequence | TTGGAAGTAAAAGTGTTTACTGGTACCAGCAGAGGCTGGG |
| | | CCAGGCCCCTGTATTGATTATCTATAGAGATAGCAACAGGC |
| | | CGACAGGGATCCCTGAGCGATTCTCTGGCGCCAGCTCGG |
| | | GGAACACGGCCACCCTGACCATCAGCGGGGCCCTGGCCG |
| | | AGGACGAGGCTGACTATTACTGCCAGGTGTGGGACAACAG |
| | | TAATAAGGCTATGTTCGGCGGAGGCACCCATCTGACCGTC |
| | | CTCG |
| 604 | PMX100 light chain | SFVLTQLPSVSVTLRQTARITCGGDSIGSKSVYWYQQRLGQA |
| | variable region amino | PVLIIYRDSNRPTGIPERFSGASSGNTATLTISGALAEDEADYY |
| | acid sequence | CQVWDNSNKAMFGGGTHLTVL |
| 605 | PMX100 heavy chain | GFTFSSY |
| | CDR1 | |
| 606 | PMX100 heavy chain | NSGGSS |
| | CDR2 | |
| 607 | PMX100 heavy chain | CASGSIWTHFDYW |
| | CDR3 | |
| 608 | PMX100 light chain | GGDSIGSKSVY |
| | CDR1 | |
| 609 | PMX100 light chain | RDSNRPT |
| | CDR2 | |
| 610 | PMX100 light chain | CQVWDNSNKAMF |
| | CDR3 | |
| 611 | PMX101 Heavy chain | GAGGTGCAGCTGGTGGAGTCTGGGGGAGACCTGGTGAGG |
| | variable region nucleic | CCTGGGGGGTCCCTGAGACTTTCCTGTGTGGCCTCTGGAT |
| | acid sequence | TCACCTTCAGTAACTACCACATGAGCTGGGTCCGCCAGGC |
| | | TCCAGGGAAGGGGCTTCAGTGGGTCGCATTCATTAACAGT |
| | | GGTGGAAGTAGCACAAGCTATGCAGACGCTGTGAGGGGC |
| | | CGATTCACCATCTCCAGAGACTACGCCAAGAACACGCTGT |
| | | ATCTTCAGATGAACAGCCTGAGAGCCGAGGACACGGCCGT |
| | | CTATTACTGTGCGAGTGGATCTATATGGACTCATTTTGACT |
| | | ATTGGGGCCAGGGAACCCTGGTCACCGTCTCCTCAG |
| 612 | PMX101 Heavy chain | EVQLVESGGDLVRPGGSLRLSCVASGFTFSNYHMSWVRQAP |
| | variable region amino | GKGLQWVAFINSGGSSTSYADAVRGRFTISRDYAKNTLYLQM |
| | acid sequence | NSLRAEDTAVYYCASGSIWTHFDYWGQGTLVTVSS |
| 613 | PMX101 light chain | TCCTTTGTGCTGACACAGCTGCCATCCGTGAGTGTGACCC |
| | variable region | TGAGGCAGACGGCCCGCATCACCTGTGGGGGAGACATCA |
| | nucleotide sequence | TTGGAAGTAAAAGTGTTTACTGGTTCCAGCAGAAGCTGGG |
| | | CCAGGCCCCTATACTGATTATCTATAGAGATGACAACAGGC |
| | | CGACAGGGATCCCTGAGCGATTCTCTGGCGCCAGCTCGG |
| | | GGAACACGGCCACCCTGATCATCAGCGGGGCCCTGGCCG |
| | | AGGACGAGGCTCACTATTACTGCCAGGTGTGGGACAGCAG |
| | | TTCTAAGGCCATGTTCGGCGGAGGCACCCATCTGACCGTC |
| | | CTCG |
| 614 | PMX101 light chain | SFVLTQLPSVSVTLRQTARITCGGDIIGSKSVYWFQQKLGQAP |
| | variable region amino | ILIIYRDDNRPTGIPERFSGASSGNTATLIISGALAEDEAHYYCQ |
| | acid sequence | VWDSSSKAMFGGGTHLTVL |
| 615 | PMX101 heavy chain | GFTFSNY |
| | CDR1 | |
| 616 | PMX101 heavy chain | NSGGSS |
| | CDR2 | |
| 617 | PMX101 heavy chain | CASGSIWTHFDYW |
| | CDR3 | |
| 618 | PMX101 light chain | GGDIIGSKSVY |
| | CDR1 | |
| 619 | PMX101 light chain | RDDNRPT |
| | CDR2 | |
| 620 | PMX101 light chain | CQVWDSSSKAMF |
| | CDR3 | |
| 621 | PMX102 Heavy chain | GAGGTACAACTGGTGGAGTCTGGGGGAGACCTGGTGAGG |
| | variable region nucleic | CCTGGGGGGTCCCTGAGACTTTCCTGTGTGGCCTCTGGAT |
| | acid sequence | TCACCTTCAATAATTACCACATGAGCTGGGTCCGCCAGGCT |
| | | CCTGGGAAGGGGCTTCAGTGGGTCGCATACATTAACAGTG |
| | | GTGGAAGTAGCACAAGTTATGCAGACGCTGTGAAGGGCCG |
| | | ATTCACCATCTCCAGAGACAACGCCAAGAACACGCTGTATC |
| | | TTCAGATGAACAGCCTGAGAGCCGAGGACACGGCCTTATA |
| | | TTATTGTGCGAGTGGATCTATATGGACTCATTTTGACTACT |
| | | GGGGCCAGGGAACCCTGGTCACCGTCTCCTCAG |
| 622 | PMX102 Heavy chain | EVQLVESGGDLVRPGGSLRLSCVASGFTFNNYHMSWVRQAP |
| | variable region amino | GKGLQWVAYINSGGSSTSYADAVKGRFTISRDNAKNTLYLQM |
| | acid sequence | NSLRAEDTALYYCASGSIWTHFDYWGQGTLVTVSS |
| 623 | PMX102 light chain | TCCTTTGTGCTGACACAGCTGCCATCCGTGAGTGTGACCC |
| | variable region | TGAGGCAGACGGCCCGCATCACCTGTGGGGGAGACATCA |
| | nucleotide sequence | TTGGAAGTAAAAATGTTTACTGGTACCAGCAGAGGCTGGG |
| | | CCAGGCCCCTGTATTGATTATCTATAGAGATAGTAACAGGC |
| | | CGACAGGGATCCCTGAGCGATTCTCTGGCGCCAGCTCGG |
| | | GGAACACGGCCACCCTGACCATCAGCGGGGCCCTGGCCG |
| | | AGGACGAGGGTGACTATTACTGCCAGGTGTGGGACAACAG |
| | | TAATAAGGCTATATTCGGCGGAGGCACCCATCTGACCGTC |
| | | CTCG |
| 624 | PMX102 light chain | SFVLTQLPSVSVTLRQTARITCGGDIIGSKNVYWYQQRLGQAP |
| | variable region amino | VLIIYRDSNRPTGIPERFSGASSGNTATLTISGALAEDEGDYYC |
| | acid sequence | QVWDNSNKAIFGGGTHLTVL |
| 625 | PMX102 heavy chain | GFTFNNY |
| | CDR1 | |
| 626 | PMX102 heavy chain | NSGGSS |
| | CDR2 | |
| 627 | PMX102 heavy chain | CASGSIWTHFDYW |
| | CDR3 | |
| 628 | PMX102 light chain | GGDIIGSKNVY |
| | CDR1 | |
| 629 | PMX102 light chain | RDSNRPT |
| | CDR2 | |
| 630 | PMX102 light chain | CQVWDNSNKAIF |
| | CDR3 | |
| 631 | PMX103 Heavy chain | GAGGTACAGCTGGTGGAGTCTGGGGGAGACCTGGTGAGG |
| | variable region nucleic | CCTGGGGGGTCCCTGAGACTCTCCTGTGTGGCCTCTGGAT |
| | acid sequence | TCACCTTCAGTAACCTCCACATGAATTGGGTCCGCCAGGC |
| | | TCCAGGGAAGGGGCTTCAGTGGGTCGCATTCATTAACAGT |
| | | GGTGGAAGTGGCACAACCTATGCAGACGTTGTGAGGGGC |
| | | CGATTCACCATCTCCAGGGACTACGCCAAAAACACACTGA |
| | | ATCTTCAGATGACTAGCCTGAGAGCCGAGGACACGGCCGT |
| | | ATATTATTGTGCGACTGGATCTATATGGACTCATTTTGACTA |
| | | TTGGGGCCAGGGAACCCTGGTCACCGTCTCCTCAG |
| 632 | PMX103 Heavy chain | EVQLVESGGDLVRPGGSLRLSCVASGFTFSNLHMNWVRQAP |
| | variable region amino | GKGLQWVAFINSGGSGTTYADVVRGRFTISRDYAKNTLNLQM |
| | acid sequence | TSLRAEDTAVYYCATGSIWTHFDYWGQGTLVTVSS |
| 633 | PMX103 light chain | TCCTTTGTGTTGACACAGCTGCCATCCGTGAGTGTGACCCT |
| | variable region | GAGGCAGACGGCCCGCATCACCTGTGGGGGAGACAGTAT |
| | nucleotide sequence | TGGAAGTAAAAGTGTTTACTGGTACCAGCAGAAGTTGGGC |
| | | CAGGCCCCTGTATTGATTATCTATAGAGATAGCAACAGGCC |
| | | GACAGGGATCCCTGAGCGAATCTCTGGCGCCAGCTCGGG |
| | | GAACACGGCCACCCTGACCATCAGCGGGGCCCTGGCCGA |
| | | GGACGAGGCTGACTATTACTGCCAGGTGTGGGACAGCAGT |
| | | AATAAGGCTATGTTCGGCGGAGGCACCCATCTGACCGTCC |
| | | TCG |
| 634 | PMX103 light chain | SFVLTQLPSVSVTLRQTARITCGGDSIGSKSVYWYQQKLGQA |
| | variable region amino | PVLIIYRDSNRPTGIPERISGASSGNTATLTISGALAEDEADYY |
| | acid sequence | CQVWDSSNKAMFGGGTHLTVL |
| 635 | PMX103 heavy chain | GFTFSNL |
| | CDR1 | |
| 636 | PMX103 heavy chain | NSGGSG |
| | CDR2 | |
| 637 | PMX103 heavy chain | CATGSIWTHFDYW |
| | CDR3 | |
| 638 | PMX103 light chain | GGDSIGSKSVY |
| | CDR1 | |
| 639 | PMX103 light chain | RDSNRPT |
| | CDR2 | |
| 640 | PMX103 light chain | CQVWDSSNKAMF |
| | CDR3 | |
| 641 | PMX104 Heavy chain | GAGGTGCAGCTGGTGGAGTCTGGGGGAGACCTGGTGAGG |
| | variable region nucleic | CCTGGGGGGTCCCTGAGACTTTCCTGTGTGGCCTCTGGAT |
| | acid sequence | TCACCTTCAGTAACTACCACATGAGCTGGGTCCGCCAGGC |
| | | TCCAGGGAAGGGGCTTCAGTGGGTCGCATTCATTAACAGT |
| | | GGTGGAACTAGTACAACCTATGCAGACGCTGTGAGGGGCC |
| | | GATTCACCACCTCCAGAGACTACGCCAAGAACACGCTGTA |
| | | TCTTCAGATGAACAGCCTGAGAGCCGAGGACACGGCCGTC |
| | | TATTACTGTACGAGTGGATCTATATGGACTCATTTTGACTAT |
| | | TGGGGCCAGGGAACCCTGGTCACCGTCTCCTCAG |
| 642 | PMX104 Heavy chain | EVQLVESGGDLVRPGGSLRLSCVASGFTFSNYHMSWVRQAP |
| | variable region amino | GKGLQWVAFINSGGTSTTYADAVRGRFTTSRDYAKNTLYLQ |
| | acid sequence | MNSLRAEDTAVYYCTSGSIWTHFDYWGQGTLVTVSS |
| 643 | PMX104 light chain | TCCTTTGTGCTGACACAGCTGCCATCCGTGAGTGTGACCC |
| | variable region | TGAGGCAGACGGCCCGCATCACCTGTGGGGGAGACATCA |
| | nucleotide sequence | TTGGAAGTAAAAGTGTTTACTGGTTCCAGCAGAAGCTGGG |
| | | CCAGGCCCCTATACTGATTATCTATAGAGATGACAACAGGC |
| | | CGACAGGGATCCCTGAGCGATTCTCTGGCGCCAGCTCGG |
| | | GGAACACGGCCACCCTGATCATCAGCGGGGCCCTGGCCG |
| | | AGGACGAGGCTCACTATTACTGCCAGGTGTGGGACAGCAG |
| | | TTCTAAGGCCATGTTCGGCGGAGGCACCCATCTGACCGTC |
| | | CTCG |
| 644 | PMX104 light chain | SFVLTQLPSVSVTLRQTARITCGGDIIGSKSVYWFQQKLGQAP |
| | variable region amino | ILIIYRDDNRPTGIPERFSGASSGNTATLIISGALAEDEAHYYCQ |
| | acid sequence | VWDSSSKAMFGGGTHLTVL |
| 645 | PMX104 heavy chain | GFTFSNY |
| | CDR1 | |
| 646 | PMX104 heavy chain | NSGGTS |
| | CDR2 | |
| 647 | PMX104 heavy chain | CTSGSIWTHFDYW |
| | CDR3 | |
| 648 | PMX104 light chain | GGDIIGSKSVY |
| | CDR1 | |
| 649 | PMX104 light chain | RDDNRPT |
| | CDR2 | |
| 650 | PMX104 light chain | CQVWDSSSKAMF |
| | CDR3 | |
| 651 | PMX105 Heavy chain | GAGGTGCAATTGGTGGAGTCTGGTGGAGACCTGGTGAAG |
| | variable region nucleic | CCTGGGGGGTCCCTGAGACTTTCCTGTGTGGCCTCTGGAT |
| | acid sequence | TCACCTTCAATACCTACCACATGAACTGGGTCCGCCAGGC |
| | | TCCAGGGAAGGGGCTTCACTGGGTCGCATTCATTAACAGC |
| | | GGTGGAAGTGGCACAAGCTATGAAGACACTGTGCAGGGC |
| | | CGATTCACCATCTCCAGAGACTACGCCAAGAACACACTCTA |
| | | TCTTCAGATGAACAGCCTGAGAGCCGAGGACACGGCCGTA |
| | | TATTACTGTGCGTGTGGATCTATATGGACTCATTTTGACTA |
| | | CTGGGGCCAGGGAACCCTGGTCACCGTCTCCTCAG |
| 652 | PMX105 Heavy chain | EVQLVESGGDLVKPGGSLRLSCVASGFTFNTYHMNWVRQAP |
| | variable region amino | GKGLHWVAFINSGGSGTSYEDTVQGRFTISRDYAKNTLYLQM |
| | acid sequence | NSLRAEDTAVYYCACGSIWTHFDYWGQGTLVTVSS |
| 653 | PMX105 light chain | TCCTATGTGCTGACACAGCTGCCATCCGTGAGTGTGACCC |
| | variable region | TGAGGCAGACGGCCCACATCACCTGTGGGGGAGACATCAT |
| | nucleotide sequence | TGGAAGTAAAAGTGTTTACTGGTACCAGCAGAAGCTGGGC |
| | | CAGGCCCCTGTAATGATTATCTATAGAGATAACAACAGGCC |
| | | GACAGGGATCCCTGAGCGATTCTCTGGCGCCAGCTCGGG |
| | | GAACACGGCCACCCTGACCATCAGCGGGTCCCTGGCCGA |
| | | GGACGAGGCTGACTATTACTGCCAGGTTTGGGACAGCAGG |
| | | ACTCAGGCTTTGTTCGGCGGAGGCACCCATCTGACCGTCC |
| | | TCG |
| 654 | PMX105 light chain | SYVLTQLPSVSVTLRQTAHITCGGDIIGSKSVYWYQQKLGQAP |
| | variable region amino | VMIIYRDNNRPTGIPERFSGASSGNTATLTISGSLAEDEADYY |
| | acid sequence | CQVWDSRTQALFGGGTHLTVL |
| 655 | PMX105 heavy chain | GFTFNTY |
| | CDR1 | |
| 656 | PMX105 heavy chain | NSGGSG |
| | CDR2 | |
| 657 | PMX105 heavy chain | CACGSIWTHFDYW |
| | CDR3 | |
| 658 | PMX105 light chain | GGDIIGSKSVY |
| | CDR1 | |
| 659 | PMX105 light chain | RDNNRPT |
| | CDR2 | |
| 660 | PMX105 light chain | CQVWDSRTQALF |
| | CDR3 | |
| 661 | PMX106 Heavy chain | GAGGTGCAGCTGGTGGAGTCTGGGGGAGACCTGGTGAAG |
| | variable region nucleic | CCTGGGGGGTCCCTGAGACTTTCCTGTGTGGCCTCTGGAT |
| | acid sequence | TCACCTTCAGTAGCTACCACATGAGCTGGGTCCGCCAGGC |
| | | TCCAGGGAAGGGGCTTCAGTGGGTCGCATACATTAACAGT |
| | | GGTGGAAGTAGCACAAGCTATGCAGACGCTGTGAAGGGC |
| | | CGATTCACCATCTCCAGAGACAACGCCAAGAACACGCTGT |
| | | ATCTTCAGATGAACAGCCTGAGAGCCGAGGACACGGCCGT |
| | | GTATTACTGTGCGGGCTTTATATGGACTCATTTTGACTACT |
| | | GGGGCCAGGGAACCCTGGTCACCGTCTCCTCAG |
| 662 | PMX106 Heavy chain | EVQLVESGGDLVKPGGSLRLSCVASGFTFSSYHMSWVRQAP |
| | variable region amino | GKGLQWVAYINSGGSSTSYADAVKGRFTISRDNAKNTLYLQM |
| | acid sequence | NSLRAEDTAVYYCAGFIWTHFDYWGQGTLVTVSS |
| 663 | PMX106 light chain | GATATTGTCATGACACAGACGCCACCGTCCCTGTCTGTCA |
| | variable region | GCCCTAGAGAGACGGCCTCCATCTCCTGCAAGGCCAGTCA |
| | nucleotide sequence | GAGCCTCCTGCACAGTGATGGAAACACCTATTTGGATTGG |
| | | TACCTGCAAAAGCCAGGCCAGTCTCCACAGCTTCTGATCTA |
| | | CTTGGTTTCCAACCGCTTCACTGGCGTGTCAGACAGGTTC |
| | | AGTGGCAGCGGGTCAGGGACAGATTTCACCCTGAGAATCA |
| | | GCAGAGTGGAGGCTAACGATACTGGAGTTTATTACTGCGG |
| | | GCAAGGTACACAGCTGTGGACGTTCGGAGCAGGAACCAA |
| | | GGTGGAGCTCAAAC |
| 664 | PMX106 light chain | DIVMTQTPPSLSVSPRETASISCKASQSLLHSDGNTYLDWYLQ |
| | variable region amino | KPGQSPQLLIYLVSNRFTGVSDRFSGSGSGTDFTLRISRVEAN |
| | acid sequence | DTGVYYCGQGTQLWTFGAGTKVELK |
| 665 | PMX106 heavy chain | GFTFSSY |
| | CDR1 | |
| 666 | PMX106 heavy chain | NSGGSS |
| | CDR2 | |
| 667 | PMX106 heavy chain | CAGFIWTHFDYW |
| | CDR3 | |
| 668 | PMX106 light chain | KASQSLLHSDGNTYLD |
| | CDR1 | |
| 669 | PMX106 light chain | LVSNRFT |
| | CDR2 | |
| 670 | PMX106 light chain | CGQGTQLWTF |
| | CDR3 | |
| 671 | PMX107 Heavy chain | GCGGTGCAGCTGGTGGAGTCTGGGGGAGACCTGGTGAGG |
| | variable region nucleic | CCTGGGGGGTCCCTGAGACTTTCCTGTGTGGCCTCTGGAT |
| | acid sequence | TCACCTTCAGTAGTTACCACATGAACTGGGTCCGCCAGGC |
| | | TCCTGGGAAGGGGCTTCAGTGGGTCGCATTCATTAACAGT |
| | | GGAGGAAGTAGTACAACCTATGCAGACGTTGTCAGGGGCC |
| | | GATTCACCATCTCCAGAGACTACGCCAAAAACACGCTATTT |
| | | CTTCAGATGAACAGCCTGAGAGCCGAGGACACGGCCGTGT |
| | | ATTATTGTGCGATTGTATCTATATGGACACATTTTGACTACT |
| | | GGGGCCAGGGAACCCTGGTCACCGTCTCCTCAG |
| 672 | PMX107 Heavy chain | AVQLVESGGDLVRPGGSLRLSCVASGFTFSSYHMNWVRQAP |
| | variable region amino | GKGLQWVAFINSGGSSTTYADVVRGRFTISRDYAKNTLFLQM |
| | acid sequence | NSLRAEDTAVYYCAIVSIWTHFDYWGQGTLVTVSS |
| 673 | PMX107 light chain | TCCTTTGTGCTGACACAGCTGCCATCCGTGAGTGTGACCC |
| | variable region | TGAGGCAGACGGCCCGCATCACCTGTGGGGGAGACATCA |
| | nucleotide sequence | TTGGAAGTAAAAATGTTTACTGGTACCAGCAGAGGCTGGG |
| | | CCAGGCCCCTGTATTGATTCTCTATAGAGATACCAACAGGC |
| | | CGACAGGGATCCCTGAGCGATTCTCTGGCGCCAGCTCGG |
| | | GGAACACGGCCACCCTGACCATCAGCGGGGCCCTGGCCG |
| | | AGGACGAGGCTGACTATTACTGCCAGGTGTGGGACAACAG |
| | | TAATAAGGCTATGTTCGGCGGAGGCACCCATCTGACCGTC |
| | | CTCG |
| 674 | PMX107 light chain | SFVLTQLPSVSVTLRQTARITCGGDIIGSKNVYWYQQRLGQAP |
| | variable region amino | VLILYRDTNRPTGIPERFSGASSGNTATLTISGALAEDEADYYC |
| | acid sequence | QVWDNSNKAMFGGGTHLTVL |
| 675 | PMX107 heavy chain | GFTFSSY |
| | CDR1 | |
| 676 | PMX107 heavy chain | NSGGSS |
| | CDR2 | |
| 677 | PMX107 heavy chain | CAIVSIWTHFDYW |
| | CDR3 | |
| 678 | PMX107 light chain | GGDIIGSKNVY |
| | CDR1 | |
| 679 | PMX107 light chain | RDTNRPT |
| | CDR2 | |
| 680 | PMX107 light chain | CQVWDNSNKAMF |
| | CDR3 | |
| 681 | PMX108 Heavy chain | GAGGTGCAGCTGGTGGAGTCTGGGGGAGACCTGGTGAGG |
| | variable region nucleic | CCGGGGGGGTCCCTGAGACTTTCCTGTGTGGCCTCTGGAT |
| | acid sequence | TCACCTTCACTAACTACCACATGAGCTGGGTCCGCCAGGC |
| | | TCCAGGGAAGGGGCTTCAGTGGGTCGCATTCATTAACAGT |
| | | GGTGGAAGTAGCACAACCTATACAGACGTTGTGAGGGGCC |
| | | GATTCACCATCTCCAGAGACTACGCCAAAAACACGCTGTAT |
| | | CTTCAGATGAACAGCCTGACAGCCGAGGACACGGCCGTGT |
| | | ATTACTGTGCGAGTGGAGCTATATGGACTCATTTTGACCAC |
| | | TGGGGCCAGGGAACCCTGGTCACCGTCTCCTCAG |
| 682 | PMX108 Heavy chain | EVQLVESGGDLVRPGGSLRLSCVASGFTFTNYHMSWVRQAP |
| | variable region amino | GKGLQWVAFINSGGSSTTYTDVVRGRFTISRDYAKNTLYLQM |
| | acid sequence | NSLTAEDTAVYYCASGAIWTHFDHWGQGTLVTVSS |
| 683 | PMX108 light chain | TCCTTTGTACTGACACAGCTGCCATCCGTGAGTGTGACCCT |
| | variable region | GAGGCAGACGGCCCGCATCACCTGTGGGGGAGACAGCGT |
| | nucleotide sequence | TGGAAGTAAAAGTGTTTACTGGTACCAGCAGAAGCTGGGC |
| | | CAGGCCCCTGTACTGATTATCTATAGAGATAGCAACAGGC |
| | | CGACAGGGATCCCTGAGCGATTCTCTGGCGCCAGCTCGG |
| | | GGAACACGGCCACCCTGACCATCAGCGGGGCCCTGGCCG |
| | | AGGACGAGGCTGACTATTACTGCCAGGTGTGGGACAACAG |
| | | TAATAAGGCTATATTCGGCGGAGGCACCCATCTGACCGTC |
| | | CTCG |
| 684 | PMX108 light chain | SFVLTQLPSVSVTLRQTARITCGGDSVGSKSVYWYQQKLGQA |
| | variable region amino | PVLIIYRDSNRPTGIPERFSGASSGNTATLTISGALAEDEADYY |
| | acid sequence | CQVWDNSNKAIFGGGTHLTVL |
| 685 | PMX108 heavy chain | GFTFTNY |
| | CDR1 | |
| 686 | PMX108 heavy chain | NSGGSS |
| | CDR2 | |
| 687 | PMX108 heavy chain | CASGAIWTHFDHW |
| | CDR3 | |
| 688 | PMX108 light chain | GGDSVGSKSVY |
| | CDR1 | |
| 689 | PMX108 light chain | RDSNRPT |
| | CDR2 | |
| 690 | PMX108 light chain | CQVWDNSNKAIF |
| | CDR3 | |
| 691 | PMX109 Heavy chain | GAGGTGCAGCTGGTGGAGTCTGGGGGAGACCTGGTGAGG |
| | variable region nucleic | CCGGGGGGGTCCCTGAGACTTTCCTGTGTGGCCTCTGGAT |
| | acid sequence | TCACCTTCACTAACTACCACATGAGCTGGGTCCGCCAGGC |
| | | TCCAGGGAAGGGGCTTCAGTGGGTCGCATTCATTAACAGT |
| | | GGTGGAAGTAGCACAACCTATACAGACGTTGTGAGGGGCC |
| | | GATTCACCATCTCCAGAGACTACGCCAAAAACACGCTGTAT |
| | | CTTCAGATGAACAGCCTGACAGCCGAGGACACGGCCGTGT |
| | | ATTACTGTGCGAGTGGATCTATATGGACTCATTTTGACCAC |
| | | TGGGGCCAGGGAACCCTGGTCACCGTCTCCTCAG |
| 692 | PMX109 Heavy chain | EVQLVESGGDLVRPGGSLRLSCVASGFTFTNYHMSWVRQAP |
| | variable region amino | GKGLQWVAFINSGGSSTTYTDVVRGRFTISRDYAKNTLYLQM |
| | acid sequence | NSLTAEDTAVYYCASGSIWTHFDHWGQGTLVTVSS |
| 693 | PMX109 light chain | TCCTTTGTACTGACACAGCTGCCATCCGTGAGTGTGACCCT |
| | variable region | GAGGCAGACGGCCCGCATCACCTGTGGGGGAGACAGCGT |
| | | TGGAAGTAAAAGTGTTTACTGGTACCAGCAGAAGCTGGGC |
| | nucleotide sequence | CAGGCCCCTGTACTGATTATCTATAGAGATAGCAACAGGC |
| | | CGACAGGGATCCCTGAGCGATTCTCTGGCGCCAGCTCGG |
| | | GGAACACGGCCACCCTGACCATCAGCGGGGCCCTGGCCG |
| | | AGGACGAGGCTGACTATTACTGCCAGGTGTGGGACAACAG |
| | | TAATAAGGCTATATTCGGCGGAGGCACCCATCTGACCGTC |
| | | CTCG |
| 694 | PMX109 light chain | SFVLTQLPSVSVTLRQTARITCGGDSVGSKSVYWYQQKLGQA |
| | variable region amino | PVLIIYRDSNRPTGIPERFSGASSGNTATLTISGALAEDEADYY |
| | acid sequence | CQVWDNSNKAIFGGGTHLTVL |
| 695 | PMX109 heavy chain | GFTFTNY |
| | CDR1 | |
| 696 | PMX109 heavy chain | NSGGSS |
| | CDR2 | |
| 697 | PMX109 heavy chain | CASGSIWTHFDHW |
| | CDR3 | |
| 698 | PMX109 light chain | GGDSVGSKSVY |
| | CDR1 | |
| 699 | PMX109 light chain | RDSNRPT |
| | CDR2 | |
| 700 | PMX109 light chain | CQVWDNSNKAIF |
| | CDR3 | |
| 701 | PMX110 Heavy chain | GAGGTGCAGCTGGTGGAGTCTGGGGGAGACCTGGTGAGG |
| | variable region nucleic | CCTGGGGGGTCCCTGAGACTTTCCTGTGTGGCCTCTGGAT |
| | acid sequence | TCACCTTCAGTAGCTACCACATGAGCTGGGTCCGCCAGGC |
| | | TCCAGGGAAGGGGCTTCAGTGGGTCGCATTCATTAACAGT |
| | | GGTGGAAGTAGCACAACCTATGCAGACGTTGTGAGGGGCC |
| | | GATTCACCATCTCCAGAGACTACGCCAAAAACACGCTGTAT |
| | | CTTCAGATGAACAGCCTGAGAGCCGAGGACACGGCCGTGT |
| | | ATTACTGTGCGAGTGGATCTATATTGACTCATTTTGACTACT |
| | | GGGGCCAGGGAACCCTGGTCACCGTCTCCTCAG |
| 702 | PMX110 Heavy chain | EVQLVESGGDLVRPGGSLRLSCVASGFTFSSYHMSWVRQAP |
| | variable region amino | GKGLQWVAFINSGGSSTTYADVVRGRFTISRDYAKNTLYLQM |
| | acid sequence | NSLRAEDTAVYYCASGSILTHFDYWGQGTLVTVSS |
| 703 | PMX110 light chain | TCCTTTGTGCTGACACAGCTGCCATCCGTGAGTGTGACCC |
| | variable region | TGAGGCAGACGGCCCGCATCACCTGTGGGGGAGACAGCG |
| | nucleotide sequence | TTGGAAGTAAAAGTGTTTACTGGTACCAGCAGAAGCTGGG |
| | | CCAGGCCCCTGTACTGATTATCTATAGAGATAGCAACAGG |
| | | CCGACAGGGATCCCTGAGCGATTCTCTGGCGCCAGCTCG |
| | | GGGAACACGGCCACCCTGACCATCAGCGGGGCCCTGGCC |
| | | GAGGACGAGGCTGACTATTACTGCCAGGTGTGGGACAACA |
| | | GTAATAAGGCTATGTTCGGCGGAGGCACCCATCTGACCGT |
| | | CCTCG |
| 704 | PMX110 light chain | SFVLTQLPSVSVTLRQTARITCGGDSVGSKSVYWYQQKLGQA |
| | variable region amino | PVLIIYRDSNRPTGIPERFSGASSGNTATLTISGALAEDEADYY |
| | acid sequence | CQVWDNSNKAMFGGGTHLTVL |
| 705 | PMX110 heavy chain | GFTFSSY |
| | CDR1 | |
| 706 | PMX110 heavy chain | NSGGSS |
| | CDR2 | |
| 707 | PMX110 heavy chain | CASGSILTHFDYW |
| | CDR3 | |
| 708 | PMX110 light chain | GGDSVGSKSVY |
| | CDR1 | |
| 709 | PMX110 light chain | RDSNRPT |
| | CDR2 | |
| 710 | PMX110 light chain | CQVWDNSNKAMF |
| | CDR3 | |
| 711 | PMX111 Heavy chain | GAGGTGCAGCTGGTGGAGTCTGGGGGAGACCTGGTGAGG |
| | variable region nucleic | CCGGGGGGGTCCCTGAGACTTTCCTGTGTGGCCTCTGGAT |
| | acid sequence | TCACCTTCAGTAGCTACCACATGAACTGGGTCCGCCAGGC |
| | | TCCAGGGAAGGGGCTTCAGTGGGTCGCATTCATTAATAGT |
| | | GGAGCAAGTAGCGCAACCTATGCAGACGTTGTGAGGGGC |
| | | CGATTCACCATCTCCAGAGACTACGCCAAAAACACGCTGTA |
| | | TCTTCAGATGAACAGCCTGAGAGCCGAGGACACGGCCGTG |
| | | TATTATTGTGCGAGTGGATCTATTTGGACTCATTTTGACTTC |
| | | TGGGGCCAGGGAACCCTGGTCACCGTCTCCTCAG |
| 712 | PMX111 Heavy chain | EVQLVESGGDLVRPGGSLRLSCVASGFTFSSYHMNWVRQAP |
| | variable region amino | GKGLQWVAFINSGASSATYADVVRGRFTISRDYAKNTLYLQM |
| | acid sequence | NSLRAEDTAVYYCASGSIWTHFDFWGQGTLVTVSS |
| 713 | PMX111 light chain | TCCTTTGTGCTGACACAGCTGCCATCCGTGAGTGTGACCC |
| | variable region | TGAGGCAGACGGCCCGCATCACCTGTGGGGGAGACAGCG |
| | nucleotide sequence | TTGGAAGTAAAAGTGTTTACTGGTACCAGCAGAAGCTGGG |
| | | CCAGGCCCCTGTACTGATTATCTATAGAGATAGCAACAGG |
| | | CCGACAGGGATCCCTGAGCGATTCTCTGGCGCCAGCTCG |
| | | GGGAACACGGCCACCCTGACCATCAGCGGGGCCCTGGCC |
| | | GAGGACGAGGCTGACTATTACTGCCAGGTGTGGGACAACA |
| | | GTAATAAGGCTATGTTCGGCGGAGGCACCCATCTGACCGT |
| | | CCTCG |
| 714 | PMX111 light chain | SFVLTQLPSVSVTLRQTARITCGGDSVGSKSVYWYQQKLGQA |
| | variable region amino | PVLIIYRDSNRPTGIPERFSGASSGNTATLTISGALAEDEADYY |
| | acid sequence | CQVWDNSNKAMFGGGTHLTVL |
| 715 | PMX111 heavy chain | GFTFSSY |
| | CDR1 | |
| 716 | PMX111 heavy chain | NSGASS |
| | CDR2 | |
| 717 | PMX111 heavy chain | CASGSIWTHFDFW |
| | CDR3 | |
| 718 | PMX111 light chain | GGDSVGSKSVY |
| | CDR1 | |
| 719 | PMX111 light chain | RDSNRPT |
| | CDR2 | |
| 720 | PMX111 light chain | CQVWDNSNKAMF |
| | CDR3 | |
| 721 | WT IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPEMLGGPS |
| | | VFIFPPKPKDTLLIARTPEVTCVVVDLDPEDPEVQISWFVDGK |
| | | QMQTAKTQPREEQFNGTYRVVSVLPIGHQDWLKGKQFTCKV |
| | | NNKALPSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 722 | Def 1 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPESLGGPS |
| | | VFIFPPKPKDTLLIARTPEVTCVVVDLDPEDPEVQISWFVDGK |
| | | QMQTAKTQPREEQFNGTYRVVSVLPIGHQDWLKGKQFTCKV |
| | | NHIGLPSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 723 | Def 2 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPESLGGPS |
| | | VFIFPPKPKDTLLIARTPEVTCVVVDLGREDPEVQISWFVDGK |
| | | QMQTAKTQPREEQFNGTYRVVSVLPIGHQDWLKGKQFTCKV |
| | | NHIGLPSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 724 | Def 3 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPESLGGPS |
| | | VFIFPPKPKDTLLIARTPEVTCVVVDLGREDPEVQISWFVDGK |
| | | QMQTAKTQPREEQFNGTYRVVSVLPIGHQDWLKGKQFTCKV |
| | | NHIGLPSSIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 725 | Def 4 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPESLGGPS |
| | | VFIFPPKPKDTLLIARTPEVTCVVVDLGREDPEVQISWFVDGK |
| | | QMQTAKTQPREEQFNGTYRVVSVLPIGHQDWLKGKQFTCKV |
| | | NHIGLGSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 726 | Def 5 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPEAAGGPS |
| | | VFIFPPKPKDTLLIARTPEVTCVVVDLDPEDPEVQISWFVDGK |
| | | QMQTAKTQPREEQFNGTYRVVSVLPIGHQDWLKGKQFTCKV |
| | | NNKALPSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 727 | Def 6 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPEAAGGPS |
| | | VFIFPPKPKDTLLIARTPEVTCVVVDLDPEDPEVQISWFVDGK |
| | | QMQTAKTQPREEQFNGTYRVVSVLPIGHQDWLKGKQFTCKV |
| | | NNKALPSSIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 728 | Def 7 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPEAAGGPS |
| | | VFIFPPKPKDTLLIARTPEVTCVVVDLDPEDPEVQISWFVDGK |
| | | QMQTAKTQPREEQFNGTYRVVSVLPIGHQDWLKGKQFTCKV |
| | | NNKALGSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 729 | Def 8 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPGMLGGPS |
| | | VFIFPPKPKDTLLIARTPEVTCVVVDLDPENPEVQISWFVDGK |
| | | QMQTAKTQPREEQFNGTYRVVSVLPIGHQDWLKGKQFTCKV |
| | | NNKALPSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 730 | Def 9 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPEAAGGPS |
| | | VFIFPPKPKDTLLIARTPEVTCVVVDLDPENPEVQISWFVDGK |
| | | QMQTAKTQPREEQFNGTYRVVSVLPIGHQDWLKGKQFTCKV |
| | | NNKALPSSIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 731 | Ofa-VH-cIgGB WT | MELGLSWIFLLAILKGVQCEVQLVESGGGLVQPGRSLRLSCA |
| | | ASGFTFNDYAMHWVRQAPGKGLEWVSTISWNSGSIGYADSV |
| | | KGRFTISRDNAKKSLYLQMNSLRAEDTALYYCAKDIQYGNYY |
| | | YGMDVWGQGTTVTVSSASTTAPSVFPLAPSCGSTSGSTVAL |
| | | ACLVSGYFPEPVTVSWNSGSLTSGVHTFPSVLQSSGLYSLSS |
| | | MVTVPSSRWPSETFTCNVAHPASKTKVDKPVPKRENGRVPR |
| | | PPDCPKCPAPEMLGGPSVFIFPPKPKDTLLIARTPEVTCVVVD |
| | | LDPEDPEVQISWFVDGKQMQTAKTQPREEQFNGTYRVVSVL |
| | | PIGHQDWLKGKQFTCKVNNKALPSPIERTISKARGQAHQPSV |
| | | YVLPPSREELSKNTVSLTCLIKDFFPPDIDVEWQSNGQQEPES |
| | | KYRTTPPQLDEDGSYFLYSKLSVDKSRWQRGDTFICAVMHEA |
| | | LHNHYTQKSLSHSPGK |
| 732 | hCD20 DNA sequence | 5′ATGACAACACCCAGAAATTCAGTAAATGGGACTTTCCCG |
| | used for generation of | GCAGAGCCAATGAAAGGCCCTATTGCTATGCAATCTGGTC |
| | target cells: | CAAAACCACTCTTCAGGAGGATGTCTTCACTGGTGGGCCC |
| | | CACGCAAAGCTTCTTCATGAGGGAATCTAAGACTTTGGGG |
| | | GCTGTCCAGATTATGAATGGGCTCTTCCACATTGCCCTGG |
| | | GGGGTCTTCTGATGATCCCAGCAGGGATCTATGCACCCAT |
| | | CTGTGTGACTGTGTGGTACCCTCTCTGGGGAGGCATTATG |
| | | TATATTATTTCCGGATCACTCCTGGCAGCAACGGAGAAAAA |
| | | CTCCAGGAAGTGTTTGGTCAAAGGAAAAATGATAATGAATT |
| | | CATTGAGCCTCTTTGCTGCCATTTCTGGAATGATTCTTTCAA |
| | | TCATGGACATACTTAATATTAAAATTTCCCATTTTTTAAAAAT |
| | | GGAGAGTCTGAATTTTATTAGAGCTCACACACCATATATTA |
| | | ACATATACAACTGTGAACCAGCTAATCCCTCTGAGAAAAAC |
| | | TCCCCATCTACCCAATACTGTTACAGCATACAATCTCTGTT |
| | | CTTGGGCATTTTGTCAGTGATGCTGATCTTTGCCTTCTTCC |
| | | AGGAACTTGTAATAGCTGGCATCGTTGAGAATGAATGGAAA |
| | | AGAACGTGCTCCAGACCCAAATCTAACATAGTTCTCCTGTC |
| | | AGCAGAAGAAAAAAAAGAACAGACTATTGAAATAAAAGAAG |
| | | AAGTGGTTGGGCTAACTGAAACATCTTCCCAACCAAAGAAT |
| | | GAAGAAGACATTGAAATTATTCCAATCCAAGAAGAGGAAGA |
| | | AGAAGAAACAGAGACGAACTTTCCAGAACCTCCCCAAGAT |
| | | CAGGAATCCTCACCAATAGAAAATGACAGCTCTCCTTAA3′ |
| 733 | IgG-B | MEFVLGWVFLVAILQGVQGEVQLVESGGDLVKPAGSLRLSCV |
| | | ASGFTFSNNAMNWVRQAPGKGLQWVAGINSGGSTASADAV |
| | | KGRFTISRDNAKNTVYLQMNSLTAEDTAVYYCAKVIGNWIATS |
| | | DLDYWGQGTLVIVSSASTTAPSVFPLAPSCGSTSGSTVALAC |
| | | LVSGYFPEPVTVSWNSGSLTSGVHTFPSVLQSSGLYSLSSMV |
| | | TVPSSRWPSETFTCNVAHPASKTKVDKPVPKRENGRVPRPP |
| | | DCPKCPAPEMLGGPSVFIFPPKPKDTLLIARTPEVTCVVVDLD |
| | | PEDPEVQISWFVDGKQMQTAKTQPREEQFNGTYRVVSVLPI |
| | | GHQDWLKGKQFTCKVNNKALPSPIERTISKARGQAHQPSVYV |
| | | LPPSREELSKNTVSLTCLIKDFFPPDIDVEWQSNGQQEPESKY |
| | | RTTPPQLDEDGSYFLYSKLSVDKSRWQRGDTFICAVMHEALH |
| | | NHYTQKSLSHSPGK |
| 734 | IgG-A | MEFVLGWVFLVAILQGVQGEVQLVESGGDLVKPAGSLRLSCV |
| | | ASGFTFSNNAMNWVRQAPGKGLQWVAGINSGGSTASADAV |
| | | KGRFTISRDNAKNTVYLQMNSLTAEDTAVYYCAKVIGNWIATS |
| | | DLDYWGQGTLVIVSSASTTAPSVFPLAPSCGSTSGSTVALAC |
| | | LVSGYFPEPVTVSWNSGSLTSGVHTFPSVLQSSGLYSLSSMV |
| | | TVPSSRWPSETFTCNVVHPASNTKVDKPVFNECRCTDTPPC |
| | | PVPEPLGGPSVLIFPPKPKDILRITRTPEVTCVVLDLGREDPEV |
| | | QISWFVDGKEVHTAKTQSREQQFNGTYRVVSVLPIEHQDWLT |
| | | GKEFKCRVNHIDLPSPIERTISKARGRAHKPSVYVLPPSPKEL |
| | | SSSDTVSITCLIKDFYPPDIDVEWQSNGQQEPERKHRMTPPQ |
| | | LDEDGSYFLYSKLSVDKSRWQQGDPFTCAVMHETLQNHYTD |
| | | LSLSHSPGK |
| 735 | IgG-C | MEFVLGWVFLVAILQGVQGEVQLVESGGDLVKPAGSLRLSCV |
| | | ASGFTFSNNAMNWVRQAPGKGLQWVAGINSGGSTASADAV |
| | | KGRFTISRDNAKNTVYLQMNSLTAEDTAVYYCAKVIGNWIATS |
| | | DLDYWGQGTLVIVSSASTTAPSVFPLAPSCGSQSGSTVALAC |
| | | LVSGYIPEPVTVSWNSGSLTSGVHTFPSILQSSGLYSLSSMVT |
| | | VPSSRWPSETFTCNVAHPATNTKVDKPVVKECECKCNCNNC |
| | | PCPGCGLLGGPSVFIFPPKPKDILVTARTPTVTCVVVDLDPEN |
| | | PEVQISWFVDSKQVQTANTQPREEQSNGTYRVVSVLPIGHQ |
| | | DWLSGKQFKCKVNNKALPSPIEEIISKTPGQAHQPNVYVLPPS |
| | | RDEMSKNTVTLTCLVKDFFPPEIDVEWQSNGQQEPESKYRM |
| | | TPPQLDEDGSYFLYSKLSVDKSRWQRGDTFICAVMHEALHN |
| | | HYTQKSLSHSPGK |
| 736 | IgG-D | MEFVLGWVFLVAILQGVQGEVQLVESGGDLVKPAGSLRLSCV |
| | | ASGFTFSNNAMNWVRQAPGKGLQWVAGINSGGSTASADAV |
| | | KGRFTISRDNAKNTVYLQMNSLTAEDTAVYYCAKVIGNWIATS |
| | | DLDYWGQGTLVIVSSASSTAPSVFPLAPSCGSTSGSTVALAC |
| | | LVSGYFPEPVTVSWNSGSLTSGVHTFPSVLKSSGLYSLSSMV |
| | | TVPSSRLPSETFTCNVVHPATNTKVDKPVPKESTCKCISPCPV |
| | | PESLGGPSVFIFPPKPKDILRITRTPEVTCVVLDLGREDPEVQI |
| | | SWFVDGKEVHTAKTQPREQQFNSTYRVVSVLPIEHQDWLTG |
| | | KEFKCRVNHIGLPSPIERTISKARGQAHQPGVYVLPPSPKELS |
| | | SSDTVTLTCLIKDFFPPEIDVEWQSNGQPEPESKYHTTAPQLD |
| | | EDGSYFLYSKLSVDKSRWQQGDPFTCAVMHEALQNHYTDLS |
| | | LSHSPGK |
| 737 | PMX154 Heavy chain | GAGGTGCAGTTGGTGGAGTCTGGGGGAGACCTTGTGAAA |
| | variable region nucleic | CCTGAGGGGTCCCTGAGACTCTCCTGTGTGGTCTCTGGCT |
| | acid sequence | TCACCTTCAGTGGCTTCGACATGAGCTGGGTCCGCCAGGC |
| | | TCCAGGGAAGGGGCTGCAGTGGGTCGCATACATTAGTACT |
| | | GATGGAAGGAGCACAGGTTACACAGACGCTGTGAAGGGC |
| | | CGATTCACCATCTCCAGAGACAATGTCAAGAACACGCTGTA |
| | | TCTGCAGATGAACAGCCTGAGAACTGAGGACACAGCCGTG |
| | | TATTACTGTGCGAAGGAAACTCGTCATTGGGGCTTTGACTA |
| | | CTGGGGCCAGGGAACCCTAGTCATCGTCTCCTCAG |
| 738 | PMX154 Heavy chain | EVQLVESGGDLVKPEGSLRLSCVVSGFTFSGFDMSWVRQAP |
| | variable region amino | GKGLQWVAYISTDGRSTGYTDAVKGRFTISRDNVKNTLYLQM |
| | acid sequence | NSLRTEDTAVYYCAKETRHWGFDYWGQGTLVIVSS |
| 739 | PMX154 light chain | TCCTATGTGTTGACACAGCTGTCATCCGTGAGTGTGAGCCT |
| | variable region | GAGGCAGACGGCCCACATCACCTGTGGGGGAGACAGCAT |
| | nucleotide sequence | TGGACGTAAAAATGTTTACTGGCACCAGCAGAAGCTGGGC |
| | | CGGGCCCCTATACTGATTATCTATGATGATAGCAGCAGGC |
| | | CGTCAGGGATCCCTGAGCGATTCTCCGGCGCCAACTCGG |
| | | GGAACACGGCCACCCTGACCATCAGCGGGGCCCTGGCCG |
| | | AGGATGAGGCTGCCTATTACTGCCAGGTGTGGGACAGCAG |
| | | TACTAGTGTGTTCGGCGGAGGCACCCATCTGACCGTCCTC |
| | | G |
| 740 | PMX154 light chain | SYVLTQLSSVSVSLRQTAHITCGGDSIGRKNVYWHQQKLGRA |
| | variable region amino | PILIIYDDSSRPSGIPERFSGANSGNTATLTISGALAEDEAAYYC |
| | acid sequence | QVWDSSTSVFGGGTHLTVL |
| 741 | PMX154 heavy chain | GFTFSGF |
| | CDR1 | |
| 742 | PMX154 heavy chain | STDGRS |
| | CDR2 | |
| 743 | PMX154 heavy chain | CAKETRHWGFDYW |
| | CDR3 | |
| 744 | PMX154 light chain | GGDSIGRKNVY |
| | CDR1 | |
| 745 | PMX154 light chain | DDSSRPS |
| | CDR2 | |
| 746 | PMX154 light chain | CQVWDSSTSVF |
| | CDR3 | |
| 747 | PMX155 Heavy chain | GAGTTGCACCTAGTGGAGTCTGGGGGAGACCTGGTGAAG |
| | variable region nucleic | CCTGCGGGGTCCCTGAGACTGTCCTGTGTGGCCTCTGGAT |
| | acid sequence | TCACCTTCAGTAGCTTCAGAATGAGTTGGGTCCGCCAGTCT |
| | | CCTGGGAAGGGACTGCAATTTGTCGCAGGTATCAACAGCG |
| | | ATGGATATAACACACAGTACAGAGACGATGTGAAGGGTCG |
| | | ATTCACCATCTCCAGAGACAACACCGAAAAGACACTATATC |
| | | TACAGATGAACAACCTGAGAGTCGAGGACACGGCCATGTA |
| | | TTACTGTGTAAAGGATCAGGGAAGTTGGGGAGGAAATTTT |
| | | GACTTCTGGGGCCAGGGAACCCTGGTCACCGTCTCCTCAG |
| 748 | PMX155 Heavy chain | ELHLVESGGDLVKPAGSLRLSCVASGFTFSSFRMSWVRQSP |
| | variable region amino | GKGLQFVAGINSDGYNTQYRDDVKGRFTISRDNTEKTLYLQM |
| | acid sequence | NNLRVEDTAMYYCVKDQGSWGGNFDFWGQGTLVTVSS |
| 749 | PMX155 light chain | TCCTATGGCCTGACTCAGCCACCATCCGTGAATGTGACCC |
| | variable region | TGAGGGAGACGGCCCACATCACCTGTGGGGGAGACAGCA |
| | nucleotide sequence | TTGGAAGTAAATATGTTCAATGGATCCAGCAGATTCCAGGC |
| | | CAGGCCCCCGTAGTGATTATCTATAAAGATAGGAACAGGC |
| | | CGACAGGGATCCCTGAGCGATTCTCTGGCGCCAACTCAGG |
| | | GAACACGGCTACCCTGACCATCAGTGGGGCCCTGGCCGA |
| | | AGACGAGGCTGACTATTACTGCCAGGTGGGGGACAGTGGT |
| | | ACTAGTGTGTTCGGCGGAGGCACCCATCTGACCGTCCTCG |
| 750 | PMX155 light chain | SYGLTQPPSVNVTLRETAHITCGGDSIGSKYVQWIQQIPGQAP |
| | variable region amino | VVIIYKDRNRPTGIPERFSGANSGNTATLTISGALAEDEADYYC |
| | acid sequence | QVGDSGTSVFGGGTHLTVL |
| 751 | PMX155 heavy chain | GFTFSSF |
| | CDR1 | |
| 752 | PMX155 heavy chain | NSDGYN |
| | CDR2 | |
| 753 | PMX155 heavy chain | CVKDQGSWGGNFDFW |
| | CDR3 | |
| 754 | PMX155 light chain | GGDSIGSKYVQ |
| | CDR1 | |
| 755 | PMX155 light chain | KDRNRPT |
| | CDR2 | |
| 756 | PMX155 light chain | CQVGDSGTSVF |
| | CDR3 | |
| 757 | PMX156 Heavy chain | GAGTTGCAGCTGGTGGAGTCTGGGGGAGACCTGGTGAAG |
| | variable region nucleic | CCTGCGGGGTCCCTGAGACTGTCCTGTGTGGCCTCTGGAT |
| | acid sequence | TCACCTTCAGTAACTTCAGAATGAGTTGGGTCCGCCAGGC |
| | | TCCTGGGAAGGGACTGCAATTTGTCGCAGGTATCAACAGC |
| | | GATGGATATAGCACACAGTACAGAGACGATGTGAAGGGTC |
| | | GATTCACCATCTCCAGAGACAACACCGAAAAGACACTATAT |
| | | CTACAGATGAACAACCTGAGAGTCGAGGACACGGCCATGT |
| | | ATTACTGTGTAAAGGATCAGGGAAGTTGGGGAGGAAATTTT |
| | | GACTACTGGGGCCAGGGAACCCTGGTCACCGTCTCCTCAG |
| 758 | PMX156 Heavy chain | ELQLVESGGDLVKPAGSLRLSCVASGFTFSNFRMSWVRQAP |
| | variable region amino | GKGLQFVAGINSDGYSTQYRDDVKGRFTISRDNTEKTLYLQM |
| | acid sequence | NNLRVEDTAMYYCVKDQGSWGGNFDYWGQGTLVTVSS |
| 759 | PMX156 light chain | TCCTATGGCCTGACTCAGCCACCATCCGTGAATGTGACCC |
| | variable region | TGAGGGAGACGGCCCACATCACCTGTGGGGGAGACAGCA |
| | nucleotide sequence | TTGGAAGTAAATATGTTCAATGGATCCAGCAGATTCCAGGC |
| | | CAGGCCCCCGTAGTGATTATCTATAAAGATAGGAACAGGC |
| | | CGACAGGGATCCCTGAGCGATTCTCTGGCGCCAACTCAGG |
| | | GAACACGGCTACCCTGACCATCAGTGGGGCCCTGGCCGA |
| | | AGACGAGGCTGACTATTACTGCCAGGTGGGGGACAGTGGT |
| | | ACTAGTGTGTTCGGCGGAGGCACCCATCTGACCGTCCTCG |
| 760 | PMX156 light chain | SYGLTQPPSVNVTLRETAHITCGGDSIGSKYVQWIQQIPGQAP |
| | variable region amino | VVIIYKDRNRPTGIPERFSGANSGNTATLTISGALAEDEADYYC |
| | acid sequence | QVGDSGTSVFGGGTHLTVL |
| 761 | PMX156 heavy chain | GFTFSNF |
| | CDR1 | |
| 762 | PMX156 heavy chain | NSDGYS |
| | CDR2 | |
| 763 | PMX156 heavy chain | CVKDQGSWGGNFDYW |
| | CDR3 | |
| 764 | PMX156 light chain | GGDSIGSKYVQ |
| | CDR1 | |
| 765 | PMX155 light chain | KDRNRPT |
| | CDR2 | |
| 766 | PMX156 light chain | CQVGDSGTSVF |
| | CDR3 | |
| 767 | PMX291 Heavy chain | GAGGTGCAGCTGGTGGAGTCTGGGGGAGACCTGTTGAAG |
| | variable region nucleic | CCTGGGGGGTCCCTGAGACTCTCCTGTGTAGCCTCTGGAT |
| | acid sequence | TCCCCTTCAGGAACTACGACATGACCTGGGTCCGCCAGGC |
| | | TCCTGGGAAGGGGCTGCAGTGGGTCGCCGCTATTCACTAT |
| | | GATGGAAGTAACACATACTACACTGACGCTGTGAAGGGCC |
| | | GATTCACCATTTCCAGAGACAACGCCAGGAACACAGTGTTT |
| | | CTGCAGATGAACAGCCTGAGAGCCGAGGACACGGCTGTG |
| | | TATTACTGTGCGAAGAAATTTAATAATTACCGTCCTTTTGAT |
| | | TACTGGGGCCAGGGAACCCTGGTCACCGTCTCCTCAG |
| 768 | PMX291 Heavy chain | EVQLVESGGDLLKPGGSLRLSCVASGFPFRNYDMTWVRQAP |
| | variable region amino | GKGLQWVAAIHYDGSNTYYTDAVKGRFTISRDNARNTVFLQM |
| | acid sequence | NSLRAEDTAVYYCAKKFNNYRPFDYWGQGTLVTVSS |
| 769 | PMX291 light chain | GATATTGTCATGACACAGACGCCACCGTCCCTGTCTGTCA |
| | variable region | GCCCTAGAGAGACGCCCTCCATCTCCTGCAAGGCCAGTCA |
| | nucleotide sequence | GAGCCTCCTGCGCAGTGATGGAAACACCTATTTGGAATGG |
| | | TACCTGCAAAAGCCCGGCCAGTCTCCACAGCTTCTGATCT |
| | | ACTTGGTTTCCAACCGCTTCACTGGCGTGTCAGACAGGTT |
| | | CAGTGGCAGCGGGTCAGGGACAGATTTCACCCTGAGAATC |
| | | AGCAGAGTGGAGGCTAACGATGCTGGAGTTTATTACTGCG |
| | | GGCAAGGTACACAGCTTCCGTGGACGTTCGGAGCAGGAA |
| | | CCAAGGTGGAGCTCAAAC |
| 770 | PMX291 light chain | DIVMTQTPPSLSVSPRETPSISCKASQSLLRSDGNTYLEWYLQ |
| | variable region amino | KPGQSPQLLIYLVSNRFTGVSDRFSGSGSGTDFTLRISRVEAN |
| | acid sequence | DAGVYYCGQGTQLPWTFGAGTKVELK |
| 771 | PMX291 heavy chain | GFPFRNY |
| | CDR1 | |
| 772 | PMX291 heavy chain | HYDGSN |
| | CDR2 | |
| 773 | PMX291 heavy chain | CAKKFNNYRPFDYW |
| | CDR3 | |
| 774 | PMX291 light chain | KASQSLLRSDGNTYLE |
| | CDR1 | |
| 775 | PMX291 light chain | LVSNRFT |
| | CDR2 | |
| 776 | PMX291 light chain | CGQGTQLPWTF |
| | CDR3 | |
| 777 | PMX292 Heavy chain | GCGGTGCAGCTGGTGGAGTCTGGGGGAGACCTGGTGAGG |
| | variable region nucleic | CCGGGGGGGTCCCTGAGACTCTCCTGTGTAGCCTCTGGAT |
| | acid sequence | TCACCTTCAGTAACTTCGACATGTACTGGGTCCGCCAGGC |
| | | TCCTGGGAGGGGGCTGCAGTGGGTCGCAACTATTTCCTAT |
| | | GATGGATTTAACACATACTCCACTGACACTGTGCGGGGCC |
| | | GATTCACCATCTCCAGAGACAACGCCAGGAACACAGTGTA |
| | | TCTGCAGATGAACAGTCTCAGAGTCGAGGACACGGCTGTT |
| | | TTTTACTGTACGGCTAGGAGGTTTGGACTTGGGTACTGGG |
| | | GCCAGGGAACCCTGGTCACCGTCTCCTCAG |
| 778 | PMX292 Heavy chain | AVQLVESGGDLVRPGGSLRLSCVASGFTFSNFDMYWVRQAP |
| | variable region amino | GRGLQWVATISYDGFNTYSTDTVRGRFTISRDNARNTVYLQM |
| | acid sequence | NSLRVEDTAVFYCTARRFGLGYWGQGTLVTVSS |
| 779 | PMX292 light chain | GATATTTTCATGGCACAGACGCCACCGTCCCTGTCTGTCA |
| | variable region | GCCCTAGAGAGACGGCCTCCATCTCCTGTAAGGCCAGTCA |
| | nucleotide sequence | GAGCCTCCTGCACAGTGATGGAAACACCTATTTGGATTGG |
| | | TACCTGCAAAAGCCAGGCCAGTCTCCACAGCTTCTGATCTA |
| | | CTTGGTTTCCAACCGCTTCCCTGGCGTGTCAGACAGGTTC |
| | | AGTGGCAGCGGGTCAGGGACAGGTTTCACCCTGAGAATCA |
| | | GCAGAGTGGAGGCTAGTGATACTGGAGTTTATTACTGCGG |
| | | GCAAGGTACACAGCTTCCTTATACCTTCAGCCAGGGAACC |
| | | AAGCTGGAGATAAAAC |
| 780 | PMX292 light chain | DIFMAQTPPSLSVSPRETASISCKASQSLLHSDGNTYLDWYLQ |
| | variable region amino | KPGQSPQLLIYLVSNRFPGVSDRFSGSGSGTGFTLRISRVEAS |
| | acid sequence | DTGVYYCGQGTQLPYTFSQGTKLEIK |
| 781 | PMX292 heavy chain | GFTFSNF |
| | CDR1 | |
| 782 | PMX292 heavy chain | SYDGFN |
| | CDR2 | |
| 783 | PMX292 heavy chain | CTARRFGLGYW |
| | CDR3 | |
| 784 | PMX292 light chain | KASQSLLHSDGNTYLD |
| | CDR1 | |
| 785 | PMX292 light chain | LVSNRFP |
| | CDR2 | |
| 786 | PMX292 light chain | CGQGTQLPYTF |
| | CDR3 | |
| 787 | PMX293 Heavy chain | GAGGTGCGACTGCTGGAGTCTGGGGGAGACCTTGTGAAA |
| | variable region nucleic | CCTGCGGGGTCCCTGAGACTCTCCTGTGCGGTTTCTGGCT |
| | acid sequence | TCACCTTCAGTACCTACGACATGAGTTGGGTCCGCCAGGC |
| | | TCCAGGGAAGGGGCTGCAGTGGGTCGCATACATTACCTAT |
| | | GATGGAAGGACCACAAGTTACACAGACACTGTGAAGGGCC |
| | | GATTCACCGTCTCCAGAGACAATGCCAAGAACATGCTGTAT |
| | | CTGAAGATGAACAGCCTGAGAAGTGAGGACGCAGCCGTTT |
| | | ATTACTGTGCGAAGGCCCATAGGGGGTGGTTTTTTGACCA |
| | | CTGGGGCCAGGGAACCCTGGTCACCGTCTCCTCAG |
| 788 | PMX293 Heavy chain | EVRLLESGGDLVKPAGSLRLSCAVSGFTFSTYDMSWVRQAP |
| | variable region amino | GKGLQWVAYITYDGRTTSYTDTVKGRFTVSRDNAKNMLYLK |
| | acid sequence | MNSLRSEDAAVYYCAKAHRGWFFDHWGQGTLVTVSS |
| 789 | PMX293 light chain | TCCTATGTGCTGACACAGCTGCCATCCGTGAGTGTGACCC |
| | variable region | TGAGGCAGACGGCCCGCATCACCTGTGGGGGAGACAGTA |
| | nucleotide sequence | TTGGAAGTAAAAATGTTTACTGGTACCAACAGAAACTGGGC |
| | | CAGGCCCCTGTATTGATTATCTATGATGATACCAGCAGGCC |
| | | GTCAGGGATCCCTGAGCGATTCTCCGGCGCCAATTCGGG |
| | | GACCACGGCCACCCTGACCATCAGCGGGGCCCTGGCCGA |
| | | GGATGAGGCTGACTATTACTGCCAGGTGTGGGACAGCAGT |
| | | ACTGGTGTGTTCGGCGGAGGCACCCACCTGACCGTCCTC |
| | | G |
| 790 | PMX293 light chain | SYVLTQLPSVSVTLRQTARITCGGDSIGSKNVYWYQQKLGQA |
| | variable region amino | PVLIIYDDTSRPSGIPERFSGANSGTTATLTISGALAEDEADYY |
| | acid sequence | CQVWDSSTGVFGGGTHLTVL |
| 791 | PMX293 heavy chain | GFTFSTY |
| | CDR1 | |
| 792 | PMX293 heavy chain | TYDGRT |
| | CDR2 | |
| 793 | PMX293 heavy chain | CAKAHRGWFFDHW |
| | CDR3 | |
| 794 | PMX293 light chain | GGDSIGSKNVY |
| | CDR1 | |
| 795 | PMX293 light chain | DDTSRPS |
| | CDR2 | |
| 796 | PMX293 light chain | CQVWDSSTGVF |
| | CDR3 | |
| 797 | PMX294 Heavy chain | GAGGTGCAGCTGGTGGAGTCTGGGGGAGACCTTGTGAAA |
| | variable region nucleic | CCTGAGGGGTCCCTGAGACTCTCCTGTGCGGTCTCTGGCT |
| | acid sequence | TCACCTTCAGTAGTAACGACATGAGTTGGGTCCGCCAGGC |
| | | TCCAGGGAAGGGGCTGCAGTGGGTCGCATACATTACCTAT |
| | | GATGGAAGGAGCACAAGTTACACAGACATTGTGAAGGGCC |
| | | GATTCACCGTCTCCAGAGACAATGCCAAGAACACGCTGTA |
| | | TCTGCAGATGAATAGCCTGAGAAGTGAGGACGCAGCCGTG |
| | | TACTACTGTGCGAAGGCCCAGAGGGGGTGGTTTTTTGACT |
| | | ACTGGGGCCAGGGAACCCTGGTCACCGTCTCCTCAG |
| 798 | PMX294 Heavy chain | EVQLVESGGDLVKPEGSLRLSCAVSGFTFSSNDMSWVRQAP |
| | variable region amino | GKGLQWVAYITYDGRSTSYTDIVKGRFTVSRDNAKNTLYLQM |
| | acid sequence | NSLRSEDAAVYYCAKAQRGWFFDYWGQGTLVTVSS |
| 799 | PMX294 light chain | TCCTATGTGCTGACACAGCTGCCATCCGTGAGTGTGACCC |
| | variable region | TGAGGCAGACGGCCCGCATCACCTGTGGGGGAGACAGTA |
| | nucleotide sequence | TTGGAAGTAAAAATGTTTACTGGTACCAACAGAAACTGGGC |
| | | CAGGCCCCTGTACTGATTATCTATGATGATGACAGCAGGC |
| | | CGTCAGGGATCCCTGAGCGAATCTCCGGCGCCAATTCGG |
| | | GGAACACGGCCACCCTGACCATCAGCGGGGCCCTGGCCG |
| | | AGGATGAGGCTGACTATTACTGCCAGGTGTGGGACAGCAG |
| | | TACTGGTGTGTTCGGCGGAGGCACCCACCTGACCGTCCTC |
| | | G |
| 800 | PMX294 light chain | SYVLTQLPSVSVTLRQTARITCGGDSIGSKNVYWYQQKLGQA |
| | variable region amino | PVLIIYDDDSRPSGIPERISGANSGNTATLTISGALAEDEADYY |
| | acid sequence | CQVWDSSTGVFGGGTHLTVL |
| 801 | PMX294 heavy chain | GFTFSSN |
| | CDR1 | |
| 802 | PMX294 heavy chain | TYDGRS |
| | CDR2 | |
| 803 | PMX294 heavy chain | CAKAQRGWFFDYW |
| | CDR3 | |
| 804 | PMX294 light chain | GGDSIGSKNVY |
| | CDR1 | |
| 805 | PMX294 light chain | DDDSRPS |
| | CDR2 | |
| 806 | PMX294 light chain | CQVWDSSTGVF |
| | CDR3 | |

TABLE 3
VH and VL gene usage

| PMX number | VH_name | VL_name | Family |
|---|---|---|---|
| PMX025 | IGHV3-5cf | IGLV3-14cf | Family 1 |
| PMX026 | IGHV3-5cf | IGLV3-14cf | Family 1 |
| PMX027 | IGHV3-5cf | IGLV3-14cf | Family 1 |
| PMX028 | IGHV3-5cf | IGLV3-21cf | Family 2 |
| PMX029 | IGHV3-5cf | IGLV3-21cf | Family 2 |
| PMX030 | IGHV3-5cf | IGLV3-24cf | Family 3 |
| PMX031 | IGHV3-5cf | IGLV3-24cf | Family 3 |
| PMX032 | IGHV3-5cf | IGLV3-21cf | Family 2 |
| PMX033 | IGHV3-5cf | IGLV3-21cf | Family 2 |
| PMX034 | IGHV3-5cf | IGLV3-21cf | Family 2 |
| PMX035 | IGHV3-8cf | IGLV3-24cf | Family 4 |
| PMX036 | IGHV3-5cf | IGLV3-21cf | Family 2 |
| PMX037 | IGHV3-8cf | IGLV3-24cf | Family 4 |
| PMX038 | IGHV3-5cf | IGLV3-21cf | Family 2 |
| PMX039 | IGHV3-18cf | IGLV3-3cf | Family 5 |
| PMX040 | IGHV3-8cf | IGLV3-24cf | Family 4 |
| PMX041 | IGHV3-18cf | IGLV3-3cf | Family 5 |
| PMX042 | IGHV3-5cf | IGLV3-21cf | Family 2 |
| PMX043 | IGHV3-18cf | IGLV3-3cf | Family 5 |
| PMX044 | IGHV3-18cf | IGLV3-3cf | Family 5 |
| PMX045 | IGHV3-5cf | IGLV3-21cf | Family 2 |
| PMX046 | IGHV3-18cf | IGLV3-3cf | Family 5 |
| PMX047 | IGHV3-19cf | IGKV2-5cf | Family 6 |
| PMX048 | IGHV3-8cf | IGLV3-24cf | Family 4 |
| PMX049 | IGHV3-8cf | IGLV3-24cf | Family 4 |
| PMX050 | IGHV3-18cf | IGLV3-3cf | Family 5 |
| PMX051 | IGHV3-44cf | IGLV3-8cf | Family 7 |
| PMX052 | IGHV3-19cf | IGKV2-4cf | Family 8 |
| PMX053 | IGHV3-19cf | IGKV2-5cf | Family 6 |
| PMX054 | IGHV3-18cf | IGLV3-24cf | Family 12 |
| PMX055 | IGHV3-18cf | IGLV3-3cf | Family 5 |
| PMX056 | IGHV3-18cf | IGLV3-3cf | Family 5 |
| PMX057 | IGHV3-18cf | IGLV3-3cf | Family 5 |
| PMX058 | IGHV3-18cf | IGLV3-3cf | Family 5 |
| PMX059 | IGHV3-18cf | IGLV3-3cf | Family 5 |
| PMX060 | IGHV3-5cf | IGLV3-3cf | Family 9 |
| PMX061 | IGHV3-5cf | IGLV3-3cf | Family 9 |
| PMX062 | IGHV3-18cf | IGLV3-3cf | Family 5 |
| PMX063 | IGHV3-5cf | IGLV3-8cf | Family 10 |
| PMX064 | IGHV3-5cf | IGLV3-21cf | Family 2 |
| PMX065 | IGHV3-8cf | IGLV3-24cf | Family 4 |
| PMX082 | IGHV3-5cf | IGLV3-21cf | Family 2 |
| PMX083 | IGHV3-5cf | IGLV3-21cf | Family 2 |
| PMX084 | IGHV3-18cf | IGLV3-24cf | Family 12 |
| PMX085 | IGHV3-18cf | IGLV3-3cf | Family 5 |
| PMX086 | IGHV3-5cf | IGLV3-8cf | Family 10 |
| PMX087 | IGHV3-5cf | IGLV3-8cf | Family 10 |
| PMX088 | IGHV3-44cf | IGLV3-8cf | Family 7 |
| PMX089 | IGHV3-5cf | IGLV3-14cf | Family 1 |
| PMX090 | IGHV3-5cf | IGLV3-14cf | Family 1 |
| PMX091 | IGHV3-5cf | IGLV3-14cf | Family 1 |
| PMX092 | IGHV3-5cf | IGLV3-14cf | Family 1 |
| PMX093 | IGHV3-5cf | IGLV3-14cf | Family 1 |
| PMX094 | IGHV3-5cf | IGLV3-8cf | Family 10 |
| PMX095 | IGHV3-5cf | IGLV3-8cf | Family 10 |
| PMX096 | IGHV3-5cf | IGLV3-8cf | Family 10 |
| PMX097 | IGHV3-5cf | IGLV3-8cf | Family 10 |
| PMX098 | IGHV3-5cf | IGLV3-8cf | Family 10 |
| PMX099 | IGHV3-5cf | IGLV3-8cf | Family 10 |
| PMX100 | IGHV3-5cf | IGLV3-8cf | Family 10 |
| PMX101 | IGHV3-5cf | IGLV3-8cf | Family 10 |
| PMX102 | IGHV3-5cf | IGLV3-8cf | Family 10 |
| PMX103 | IGHV3-5cf | IGLV3-8cf | Family 10 |
| PMX104 | IGHV3-5cf | IGLV3-8cf | Family 10 |
| PMX105 | IGHV3-5cf | IGLV3-8cf | Family 10 |
| PMX106 | IGHV3-5cf | IGKV2-4cf | Family 11 |
| PMX107 | IGHV3-5cf | IGLV3-8cf | Family 10 |
| PMX108 | IGHV3-5cf | IGLV3-8cf | Family 10 |
| PMX109 | IGHV3-5cf | IGLV3-8cf | Family 10 |
| PMX110 | IGHV3-5cf | IGLV3-8cf | Family 10 |
| PMX111 | IGHV3-5cf | IGLV3-8cf | Family 10 |
| PMX154 | IGHV3-18cf | IGLV3-24cf | Family 12 |
| PMX155 | IGHV3-19cf | IGLV3-21cf | Family 13 |
| PMX156 | IGHV3-19cf | IGLV3-21cf | Family 13 |
| PMX291 | IGHV3-41cf | IGKV2-4cf | Family 14 |
| PMX292 | IGHV3-41cf | IGKV2-4cf | Family 14 |
| PMX293 | IGHV3-18cf | IGLV3-24cf | Family 12 |
| PMX294 | IGHV3-18cf | IGLV3-24cf | Family 12 |

TABLE 4
Geomeans obtained from cell binding assay

| | GEO mean OX40L | GEO mean | GEO mean |
|---|---|---|---|
| Antibody | expressing HEK | WT HEK | difference |
| PMX025 | 2830000 | 18587 | 2811413 |
| PMX026 | 1390000 | 26716 | 1363284 |
| PMX027 | 1650000 | 33774 | 1616226 |
| PMX028 | 1050000 | 30825 | 1019175 |
| PMX029 | 1090000 | 28348 | 1061652 |
| PMX030 | 1420000 | 29779 | 1390221 |
| PMX031 | 1550000 | 39212 | 1510788 |
| PMX032 | 1450000 | 28489 | 1421511 |
| PMX033 | 1460000 | 28385 | 1431615 |
| PMX034 | 1390000 | 27398 | 1362602 |
| PMX035 | 1410000 | 26814 | 1383186 |
| PMX036 | 1310000 | 30928 | 1279072 |
| PMX037 | 1420000 | 27618 | 1392382 |
| PMX038 | 1560000 | 27561 | 1532439 |
| PMX039 | 1520000 | 29268 | 1490732 |
| PMX040 | 1180000 | 27194 | 1152806 |
| PMX041 | 1560000 | 28620 | 1531380 |
| PMX042 | 1510000 | 28750 | 1481250 |
| PMX043 | 1280000 | 33769 | 1246231 |
| PMX044 | 1530000 | 28856 | 1501144 |
| PMX045 | 1470000 | 28564 | 1441436 |
| PMX046 | 1250000 | 28149 | 1221851 |
| PMX047 | 1950000 | 28632 | 1921368 |
| PMX048 | 3920000 | 23672 | 3896328 |
| PMX049 | 3500000 | 24403 | 3475597 |
| PMX050 | 3080000 | 23859 | 3056141 |
| PMX051 | 3940000 | 24704 | 3915296 |
| PMX052 | 765000 | 30802 | 734198 |
| PMX053 | 3030000 | 30828 | 2999172 |
| PMX054 | 3270000 | 30515 | 3239485 |
| PMX055 | 3260000 | 30461 | 3229539 |
| PMX056 | 3160000 | 31120 | 3128880 |
| PMX057 | 3320000 | 29889 | 3290111 |
| PMX058 | 3340000 | 29794 | 3310206 |
| PMX059 | 3140000 | 29668 | 3110332 |
| PMX060 | 3100000 | 29772 | 3070228 |
| PMX061 | 3290000 | 29916 | 3260084 |
| PMX062 | 2810000 | 29671 | 2780329 |
| PMX063 | 3180000 | 30351 | 3149649 |
| PMX064 | 3450000 | 29540 | 3420460 |
| PMX065 | 3550000 | 29783 | 3520217 |
| PMX082 | 544000 | 12535 | 531465 |
| PMX083 | 701000 | 11438 | 689562 |
| PMX084 | 622000 | 12275 | 609725 |
| PMX085 | 839000 | 12581 | 826419 |
| PMX086 | 857000 | 7973 | 849027 |
| PMX087 | 1050000 | 7984 | 1042016 |
| PMX088 | 783000 | 8225 | 774775 |
| PMX105 | 584000 | 3533 | 580467 |
| PMX106 | 7759 | 3656 | 4103 |
| PMX107 | 58797 | 3625 | 55172 |
| PMX108 | 611000 | 3520 | 607480 |
| PMX109 | 633000 | 3599 | 629401 |
| PMX110 | 31927 | 3525 | 28402 |
| PMX111 | 647000 | 3572 | 643428 |
| PMX154 | 789000 | 8196 | 780804 |
| PMX155 | 738000 | 7995 | 730005 |
| PMX156 | 680000 | 7941 | 672059 |
| PMX291 | 463000 | 3826 | 459174 |
| PMX292 | 471000 | 3757 | 467243 |
| PMX293 | 602000 | 3571 | 598429 |
| PMX294 | 595000 | 3543 | 591457 |
| neg CTR | 19634 | 24019 | −4385 |

TABLE 5
Normalised HEK blue assay results, showing the percentage of OX40 signal inhibition.

| | % Signal Inhibition | | | | |
|---|---|---|---|---|---|
| Ab | Rep1 | Rep2 | Rep3 | Average | SD |
| PMX25 | 87.86279683 | 61.7414248 | 68.33773087 | 72.6473175 | 13.58347974 |
| PMX26 | 67.6056338 | 65.14084507 | 68.66197183 | 67.13615023 | 1.806901915 |
| PMX27 | 72.17741935 | 83.46774194 | 85.48387097 | 80.37634409 | 7.171678264 |
| PMX28 | 61.69354839 | 75.40322581 | 62.09677419 | 66.39784946 | 7.801490248 |
| PMX29 | 75.80645161 | 76.20967742 | 56.0483871 | 69.35483871 | 11.52548865 |
| PMX30 | 70.56451613 | 83.87096774 | 42.33870968 | 65.59139785 | 21.20804197 |
| PMX31 | 83.45070423 | 89.08450704 | 62.67605634 | 78.40375587 | 13.90882213 |
| PMX32 | 76.76056338 | 57.3943662 | 75.70422535 | 69.95305164 | 10.8889576 |
| PMX33 | 55.81395349 | 69.76744186 | 55.42635659 | 60.33591731 | 8.170238632 |
| PMX34 | 76.35658915 | 64.72868217 | 51.5503876 | 64.2118863 | 12.41117308 |
| PMX35 | 85.27131783 | 67.44186047 | 67.82945736 | 73.51421189 | 10.18379659 |
| PMX36 | 87.59689922 | 79.06976744 | 65.50387597 | 77.39018088 | 11.14186598 |
| PMX37 | 85.65891473 | 79.84496124 | 69.76744186 | 78.42377261 | 8.04049503 |
| PMX38 | 79.83870968 | 75 | 50.80645161 | 68.5483871 | 15.55427542 |
| PMX39 | 81.69014085 | 86.61971831 | 73.5915493 | 80.63380282 | 6.578007638 |
| PMX40 | 75.35211268 | 84.15492958 | 71.83098592 | 77.11267606 | 6.347801541 |
| PMX41 | 86.36990852 | 78.19287576 | 75.63755302 | 80.0667791 | 5.606201827 |
| PMX42 | 77.82258065 | 87.5 | 70.96774194 | 78.76344086 | 8.306190621 |
| PMX43 | 69.15888 | 77.33333 | 96.052632 | 80.84828067 | 13.78711836 |
| PMX44 | 75.70093 | 66 | 83.55263 | 75.08452 | 8.79253524 |
| PMX45 | 79.92957746 | 81.69014085 | 66.1971831 | 75.93896714 | 8.482432613 |
| PMX46 | 84.83671488 | 69.50477845 | 100.1686513 | 84.83671488 | 15.33193642 |
| PMX47 | 49.3485391 | 73.12009148 | 62.28748533 | 61.58537197 | 11.90131917 |
| PMX48 | 57.47664 | 69.33333 | 82.89474 | 69.90157 | 12.71857399 |
| PMX49 | 84.57944 | 79.33333 | 90.131579 | 84.68144967 | 5.399847206 |
| PMX50 | 68.69159 | 83.33333 | 77.63158 | 76.55216667 | 7.380310794 |
| PMX51 | 82.24299 | 75.33333 | 94.736842 | 84.10438733 | 9.834768401 |
| PMX52 | 15.6626506 | 29.18340027 | 21.68674699 | 22.17759929 | 6.773726438 |
| PMX53 | 24.49799197 | 45.78313253 | 48.86211513 | 39.71441321 | 13.26742778 |
| PMX54 | 21.01740295 | 32.7978581 | 42.03480589 | 31.95002231 | 10.53432133 |
| PMX55 | 48.32663989 | 57.69745649 | 52.34270415 | 52.78893351 | 4.701318051 |
| PMX56 | 27.30923695 | 57.02811245 | 48.4605087 | 44.2659527 | 15.29701328 |
| PMX57 | 52.07496653 | 72.02141901 | 73.89558233 | 65.99732262 | 12.09347433 |
| PMX58 | 12.4497992 | 35.34136546 | 39.49129853 | 29.0941544 | 14.56301501 |
| PMX59 | 51.13788487 | 64.25702811 | 57.96519411 | 57.78670237 | 6.561392711 |
| PMX60 | 58.90227577 | 68.40696118 | 71.08433735 | 66.13119143 | 6.401952986 |
| PMX61 | 51.00401606 | 61.57965194 | 55.68942436 | 56.09103079 | 5.299243752 |
| PMX62 | 4.149933066 | 22.08835341 | 18.7416332 | 14.99330656 | 9.538563602 |
| PMX63 | 68.27309237 | 82.99866131 | 72.02141901 | 74.43105756 | 7.652801032 |
| PMX64 | 88.88888889 | 94.77911647 | 89.69210174 | 91.1200357 | 3.194204403 |
| PMX65 | 62.65060241 | 85.27443106 | 69.87951807 | 72.60151718 | 11.55492817 |
| PMX82 | 72.78911565 | 71.88208617 | 72.10884354 | 72.26001512 | 0.4720331065 |
| PMX83 | 65.98639456 | 65.75963719 | 81.63265306 | 71.12622827 | 9.09953714 |
| PMX84 | 47.16553288 | 66.89342404 | 68.02721088 | 60.69538927 | 11.73090485 |
| PMX85 | 43.76417234 | 56.23582766 | 40.81632653 | 46.93877551 | 8.185281473 |
| PMX154 | 62.57309942 | 77.19298246 | 51.4619883 | 63.74269006 | 12.90530789 |
| PMX155 | 19.88304094 | 37.42690058 | 40.93567251 | 32.74853801 | 11.2791237 |
| PMX156 | 21.6374269 | 34.50292398 | 49.12280702 | 35.0877193 | 13.75201873 |
| PMX086 | 5.847953216 | 35.0877193 | 14.03508772 | 18.32358674 | 15.08424337 |
| PMX087 | 29.8245614 | 46.19883041 | 21.6374269 | 32.55360624 | 12.50605453 |
| PMX088 | 16.95906433 | 3.50877193 | 1.169590643 | 7.212475634 | 8.521439764 |
| PMX089 | 43.2748538 | 49.70760234 | 49.12280702 | 47.36842105 | 3.55717107 |
| PMX092 | 33.33333333 | 39.18128655 | 31.57894737 | 34.69785575 | 3.980619465 |
| PMX093 | 0 | −0.5847953216 | 16.95906433 | 5.458089669 | 9.964427227 |
| PMX094 | 38.59649123 | 54.97076023 | 42.69005848 | 45.41910331 | 8.521439764 |
| PMX095 | 40.93567251 | 52.63157895 | 57.30994152 | 50.29239766 | 8.434038071 |
| PMX096 | 36.84210526 | 59.06432749 | 54.38596491 | 50.09746589 | 11.71538339 |
| PMX097 | 56.14035088 | 73.0994152 | 53.21637427 | 60.81871345 | 10.73541506 |
| PMX098 | 40.35087719 | 54.97076023 | 39.76608187 | 45.02923977 | 8.614573019 |
| PMX099 | 25.14619883 | 32.74853801 | 16.37426901 | 24.75633528 | 8.194093395 |
| PMX100 | 49.12280702 | 28.07017544 | 19.88304094 | 32.35867446 | 15.08424337 |
| PMX101 | 62.57309942 | 50.29239766 | 44.44444444 | 52.43664717 | 9.252588137 |
| PMX103 | 57.30994152 | 26.31578947 | 59.06432749 | 47.56335283 | 18.42182615 |
| PMX104 | 30.99415205 | 28.07017544 | 33.91812865 | 30.99415205 | 2.923976608 |
| PMX105 | 85.43956044 | 94.78021978 | 72.52747253 | 84.24908425 | 11.17403755 |
| PMX106 | −9.89010989 | −4.120879121 | −37.63736264 | −17.21611722 | 17.91902493 |
| PMX107 | 7.142857143 | 20.87912088 | −43.95604396 | −5.311355311 | 34.16475092 |
| PMX108 | 95.6043956 | 89.83516484 | 61.26373626 | 82.23443223 | 18.38881652 |
| PMX109 | 67.85714286 | 69.50549451 | 22.52747253 | 53.2967033 | 26.65967812 |
| PMX110 | 55.21978022 | 43.13186813 | 13.73626374 | 37.36263736 | 21.33503112 |
| PMX111 | 44.23076923 | 37.36263736 | 35.16483516 | 38.91941392 | 4.729212943 |
| PMX291 | 95.87912088 | 70.32967033 | 53.57142857 | 73.26007326 | 21.30553104 |
| PMX292 | 97.52747253 | 54.3956044 | 62.91208791 | 71.61172161 | 22.84408527 |
| PMX293 | 81.59340659 | 77.47252747 | 62.08791209 | 73.71794872 | 10.28050219 |
| PMX294 | 93.95604396 | 88.73626374 | 53.02197802 | 78.57142857 | 22.27986409 |

TABLE 6
Calculated IFN-γ levels in supernatant from PBMC activated
with CD3 and CD28 antibodies in presence of HEK cells expressing
OX40L and the indicated anti-OX40L antibody.

| | | | | | SD | | |
|---|---|---|---|---|---|---|---|
| | | mean | | | IFNg | | IFNg |
| | | IFNg at | IFNg at | IFNg at | at | IFNg at | at |
| Exp | Antibody | 10 ug/ml | 1 ug/ml | 0.1 ug/ml | 10 ug/ml | 1 ug/ml | 0.1 ug/ml |
| 1 | PMX26 | 1814.78 | 3433.28 | 5414.20 | 45.21 | 1375.01 | 3356.63 |
| | PMX27 | 1990.07 | 3288.97 | 4044.77 | 208.16 | 588.51 | 722.95 |
| | PMX28 | 3747.50 | 4167.48 | | 1137.43 | 1708.61 | |
| | PMX29 | 4098.23 | 4809.27 | | 255.24 | 1688.96 | |
| | PMX30 | 3576.96 | 3853.98 | 5362.51 | 532.12 | 264.01 | 780.97 |
| | PMX31 | 2912.32 | 3002.01 | 4036.50 | 347.72 | 287.54 | 1033.25 |
| | PMX32 | 2024.93 | 3618.68 | 4223.01 | 767.06 | 1546.09 | 2435.17 |
| | PMX33 | 2531.64 | 3951.58 | 4723.81 | 906.87 | 1184.23 | 1455.97 |
| | PMX34 | 2556.40 | 5339.85 | 7042.10 | 1272.17 | 3076.37 | 4007.35 |
| | PMX35 | 4176.35 | 3976.67 | 4495.16 | 956.20 | 709.57 | 419.94 |
| | PMX36 | 2326.33 | 4754.37 | 5649.25 | 1129.18 | 2516.11 | 1417.65 |
| | PMX37 | 2461.41 | 3790.18 | 4648.46 | 1804.94 | 1137.25 | 1786.08 |
| | PMX38 | 2442.77 | 4449.14 | 4525.85 | 1295.10 | 1040.33 | 1710.84 |
| | PMX39 | 2933.59 | 3249.37 | 4135.69 | 806.02 | 493.70 | 852.67 |
| | PMX40 | 3645.35 | 3412.83 | 3715.94 | 91.46 | 195.43 | 339.31 |
| | PMX41 | 2723.49 | 4487.53 | 5710.89 | 2252.21 | 1492.53 | 3591.86 |
| | PMX42 | 2905.62 | 3655.92 | 3704.68 | 191.37 | 272.16 | 398.88 |
| | PMX43 | 2528.79 | 3085.59 | 3545.46 | 379.45 | 345.11 | 759.76 |
| | PMX44 | 4019.73 | 5116.65 | 6095.48 | 2087.93 | 2502.78 | 3813.82 |
| | PMX45 | 3053.12 | 3325.59 | 3590.83 | 215.13 | 161.04 | 260.98 |
| | PMX46 | 2645.25 | 3882.33 | 3866.09 | 716.92 | 1161.45 | 1534.08 |
| | PMX48 | 2652.66 | 3832.05 | 4265.84 | 713.49 | 1120.59 | 1425.55 |
| | PMX49 | 2470.91 | 4239.59 | 4974.37 | 1049.96 | 1745.68 | 2370.81 |
| | PMX50 | 1899.51 | 4270.99 | 5333.26 | 659.02 | 1613.99 | 1699.31 |
| | PMX51 | 1596.68 | 3469.98 | 4544.13 | 1006.82 | 1168.05 | 180.60 |
| | | | | | mean | sd | |
| | | | | Activated PBMC | 885.59 | 184.72 | |
| | | | | only | | | |
| | | | | Activated PBMC | 2459.88 | 365.80 | |
| | | | | plus HEK WT | | | |
| | | | | Activated PBMC | 3407.36 | 865.71 | |
| | | | | plus HEK OX40L | | | |
| 2 | PMX25 | 2770.11 | 3057.09 | 3805.02 | 1045.67 | 681.15 | 1274.56 |
| | PMX47 | 2768.58 | 4677.64 | 5507.66 | 769.44 | 2263.08 | 3049.61 |
| | | | | | mean | sd | |
| | | | | Activated PBMC | 386.55 | 168.92 | |
| | | | | only | | | |
| | | | | Activated PBMC | 1817.34 | 250.69 | |
| | | | | plus HEK WT | | | |
| | | | | Activated PBMC | 4212.20 | 968.56 | |
| | | | | plus HEK OX40L | | | |
| 3 | PMX52 | 1315.32 | 3054.47 | 4681.29 | 269.15 | 526.97 | 217.93 |
| | PMX53 | 1737.69 | 2811.98 | 3777.63 | 399.51 | 690.91 | 302.86 |
| | PMX54 | 1368.39 | 2407.42 | 4081.28 | 374.45 | 209.24 | 918.24 |
| | PMX55 | 882.63 | 2862.91 | 3709.13 | 442.93 | 210.87 | 331.55 |
| | PMX56 | 1323.43 | 2611.83 | 3784.45 | 475.85 | 267.59 | 580.60 |
| | PMX57 | 1050.92 | 2394.91 | 3696.83 | 189.37 | 322.04 | 349.06 |
| | PMX58 | 1341.49 | 2542.25 | 4250.69 | 197.27 | 159.80 | 1123.55 |
| | PMX59 | 1317.75 | 3125.89 | 3563.65 | 243.01 | 812.81 | 1050.36 |
| | PMX60 | 1115.59 | 2097.85 | 3075.43 | 274.87 | 185.73 | 459.90 |
| | PMX61 | 1192.01 | 2321.95 | 3470.64 | 631.57 | 401.53 | 608.19 |
| | PMX62 | 1701.95 | 3331.77 | 4447.21 | 563.04 | 576.85 | 810.82 |
| | PMX63 | 731.17 | 2591.77 | 3520.33 | 35.02 | 345.75 | 1022.69 |
| | PMX64 | 1409.13 | 2188.59 | 3356.51 | 217.58 | 115.17 | 1339.26 |
| | PMX65 | 1961.45 | 2775.70 | 3712.55 | 221.52 | 173.26 | 947.67 |
| | | | | | mean | sd | |
| | | | | Activated PBMC | 403.44 | 273.42 | |
| | | | | only | | | |
| | | | | Activated PBMC | 1936.31 | 591.88 | |
| | | | | plus HEK WT | | | |
| | | | | Activated PBMC | 3340.52 | 596.95 | |
| | | | | plus HEK OX40L | | | |
| 4 | PMX82 | 651.66 | 1484.65 | 1743.93 | 556.18 | 199.46 | 67.16 |
| | PMX83 | 772.03 | 1318.61 | 1512.28 | 100.81 | 164.38 | 62.11 |
| | PMX84 | 805.25 | 1338.86 | 1732.75 | 130.18 | 274.27 | 225.94 |
| | PMX85 | 492.34 | 1303.38 | 1752.97 | 121.61 | 168.09 | 173.12 |
| | | | | | mean | sd | |
| | | | | Activated PBMC | 285.11 | 92.85 | |
| | | | | only | | | |
| | | | | Activated PBMC | 1019.26 | 60.26 | |
| | | | | plus HEK WT | | | |
| | | | | Activated PBMC | 1817.28 | 174.80 | |
| | | | | plus HEK OX40L | | | |
| 5 | PMX025 | 1359.97 | 2746.66 | 3081.29 | 413.02 | 634.79 | 621.45 |
| | PMX033 | 1340.55 | 2058.00 | 2654.86 | 166.80 | 667.24 | 428.35 |
| | PMX034 | 1016.67 | 1326.64 | 2383.32 | 320.74 | 254.87 | 622.78 |
| | PMX036 | 1198.65 | 1464.58 | 1900.32 | 17.46 | 219.68 | 539.28 |
| | PMX037 | 1670.23 | 1755.54 | 1974.41 | 352.94 | 323.52 | 510.13 |
| | PMX041 | 1674.07 | 2323.12 | 2114.73 | 445.58 | 665.34 | 125.76 |
| | PMX044 | 1224.32 | 2098.99 | 2491.96 | 334.83 | 488.84 | 573.10 |
| | PMX046 | 1639.40 | 2471.90 | 3456.61 | 454.11 | 541.02 | 386.48 |
| | PMX048 | 1544.70 | 1904.61 | 2996.43 | 136.77 | 117.45 | 395.65 |
| | PMX049 | 1582.82 | 1922.43 | 2551.69 | 316.23 | 175.51 | 277.21 |
| | PMX050 | 1497.70 | 2481.89 | 2723.57 | 270.41 | 303.37 | 315.16 |
| | PMX051 | 958.94 | 1635.98 | 2861.09 | 139.68 | 162.57 | 68.92 |
| | PMX053 | 1545.71 | 3434.39 | 3427.15 | 80.61 | 634.34 | 311.61 |
| | PMX063 | 1112.67 | 2394.40 | 2979.97 | 81.81 | 215.38 | 576.27 |
| | | | | | mean | sd | |
| | | | | Activated PBMC | 742.35 | 45.45 | |
| | | | | only | | | |
| | | | | Activated PBMC | 1481.56 | 161.15 | |
| | | | | plus HEK WT | | | |
| | | | | Activated PBMC | 2144.71 | 523.58 | |
| | | | | plus HEK OX40L | | | |

TABLE 6
Experiment 6

| | mean | | | SD | | |
|---|---|---|---|---|---|---|
| | IFNg at | IFNg at | IFNg at 0.1 | IFNg at | IFNg at | IFNg at |
| Antibody | 10 ug/ml | 1 ug/ml | ug/ml | 10 ug/ml | 1 ug/ml | 0.1 ug/ml |
| PMX044 | 1722.38 | 1759.58 | 2329 | 278.952 | 187.664 | 224.124 |
| PMX050 | 1245.66 | 1719.77 | 2531.82 | 42.7925 | 219.931 | 113.751 |
| PMX051 | 1305.74 | 1354.63 | 2365.89 | 252.408 | 324.315 | 46.1784 |
| PMX063 | 1102.77 | 1113.98 | 2113.32 | 205.53 | 654.097 | 132.529 |
| PMX097 | 1336.77 | 1383.23 | 2310.45 | 588.363 | 174.174 | 48.2587 |
| PMX098 | 1362.69 | 1461.92 | 2362.47 | 238.854 | 199.5 | 109.641 |
| PMX154 | 1345.93 | 1273.41 | 2521.93 | 549.126 | 249.381 | 340.828 |
| PMX155 | 987.95 | 1199.91 | 1899.39 | 217.595 | 326.565 | 485.313 |
| | | | | mean | sd | |
| | | | Activated PBMC only | 1178.41 | 117.19 | |
| | | | Activated PBMC plus HEK | 1887.27 | 556.10 | |
| | | | WT | | | |
| | | | Activated PBMC plus HEK | 2498.20 | 149.14 | |
| | | | OX40L | | | |

TABLE 6
Experiment 7

| | mean | | | SD | | |
|---|---|---|---|---|---|---|
| | IFNg at | IFNg at | IFNg at 0.1 | IFNg at | IFNg at | IFNg at |
| Antibody | 10 ug/ml | 1 ug/ml | ug/ml | 10 ug/ml | 1 ug/ml | 0.1 ug/ml |
| PMX051 | 1536.3 | 1815.21 | 1875.8 | 96.6475 | 592.227 | 627.987 |
| PMX063 | 1738 | 2020.37 | 2575.4 | 73.6416 | 280.029 | 371.363 |
| PMX097 | 1318.61 | 1561.57 | 1557.5 | 227.078 | 276.753 | 149.508 |
| PMX154 | 1764.41 | 2285.1 | 2407.64 | 143.011 | 402.333 | 443.682 |
| PMX155 | 1192.73 | 1475.6 | 1518.13 | 210.448 | 124.464 | 281.658 |
| PMX156 | 1350.71 | 1418.98 | 1626.17 | 592.994 | 693.986 | 1140.91 |
| | | | | mean | sd | |
| | | | Activated PBMC only | 2494.90 | 343.44 | |
| | | | Activated PBMC plus HEK | 1732.29 | 594.21 | |
| | | | WT | | | |
| | | | Activated PBMC plus HEK | 2522.38 | 681.991 | |
| | | | OX40L | | | |

TABLE 6
Experiment 8

| | IFNg at | IFNg at | IFNg at | IFNg at 10 | IFNg at | IFNg at |
|---|---|---|---|---|---|---|
| Antibody | 10 ug/ml | 1 ug/ml | 0.1 ug/ml | ug/ml | 1 ug/ml | 0.1 ug/ml |
| PMX155 | 357.9 | 913.51 | 992.1 | 152.3108213 | 277.3656686 | 113.2125951 |
| PMX156 | 356.41 | 1040.08 | 911.54 | 44.25830092 | 353.2083203 | 263.9567016 |
| PMX154 | 298.13 | 553.2 | 908.56 | 119.0447945 | 133.027378 | 31.03897389 |
| PMX086 | 603.78 | 629.04 | 796.25 | 115.4089823 | 269.0244727 | 35.34964215 |
| PMX087 | 486.52 | 778.08 | 959.18 | 159.0133985 | 47.25368557 | 330.1825272 |
| PMX088 | 548.81 | 511.83 | 797.76 | 174.4212418 | 110.5179031 | 153.3083628 |
| PMX089 | 392.55 | 597.97 | 628.25 | 113.3400596 | 48.49787727 | 160.525939 |
| PMX099 | 456.31 | 475.78 | 389.09 | 74.26587305 | 107.6157177 | 191.4946819 |
| PMX100 | 466.78 | 419.54 | 437.56 | 152.4082006 | 117.8966615 | 59.98653099 |
| PMX101 | 439.32 | 373.57 | 482.51 | 190.5790206 | 81.896249 | 99.86248995 |
| PMX092 | 428.95 | 482.03 | 414.24 | 64.19260004 | 116.2632276 | 164.677411 |
| PMX093 | 185.73 | 308.17 | | 134.5765626 | 10.13415512 | |
| PMX097 | 207.72 | 523.94 | | 19.93420427 | 107.4632384 | |
| PMX098 | 232.29 | 284.08 | 389.82 | 69.03847406 | 11.403802 | 206.5741751 |
| | | | | | mean | sd |
| | | | | Activated PBMC only | 165.552 | 47.70432339 |
| | | | | Activated PBMC plus | 453.67 | 98.27820267 |
| | | | | HEK WT | | |
| | | | | Activated PBMC plus | 682.185 | 87.44989348 |
| | | | | HEK OX40L | | |

TABLE 6
Experiment 9

| | IFNg at | IFNg at | IFNg at | IFNg at | IFNg at | IFNg at |
|---|---|---|---|---|---|---|
| Antibody | 10 ug/ml | 1 ug/ml | 0.1 ug/ml | 10 ug/ml | 1 ug/ml | 0.1 ug/ml |
| PMX097 | 951.94 | 1177.41 | 1335.68 | 145.4115267 | 251.8712832 | 80.03178181 |
| PMX063 | 870.03 | 1500.67 | 1455.14 | 178.699512 | 127.4849685 | 189.0959383 |
| PMX105 | 1104.64 | 1115.07 | 1326.7 | 399.3461607 | 180.9313431 | 193.302486 |
| PMX294 | 932.35 | 1308.39 | 1646.86 | 74.29910094 | 56.79637048 | 144.9110255 |
| PMX293 | 1034.17 | 1230.16 | 1651.52 | 125.7269374 | 179.1317669 | 250.3058343 |
| PMX291 | 983.92 | 1364.04 | 1442.19 | 169.5388902 | 261.9525531 | 176.134747 |
| PMX292 | 962.85 | 1316.63 | 1258.25 | 210.8505326 | 329.8375541 | 234.001241 |
| | | | | | mean | sd |
| | | | | Activated PBMC only | 498.05 | 26.86289076 |
| | | | | Activated PBMC plus | 396.12 | 103.2492266 |
| | | | | HEK WT | | |
| | | | | Activated PBMC plus | 1683.16 | 23.41211866 |
| | | | | HEK OX40L | | |

Experiment 10

| | IFNg at | IFNg at | IFNg at | IFNg at | IFNg at | IFNg at |
|---|---|---|---|---|---|---|
| Antibody | 10 ug/ml | 1 ug/ml | 0.1 ug/ml | 10 ug/ml | 1 ug/ml | 0.1 ug/ml |
| PMX105 | 193.7766667 | 302.6883333 | 652.1316667 | 35.68407535 | 128.4039884 | 73.91448054 |
| PMX108 | 413.9133333 | 534.4916667 | 687.6116667 | 23.13906779 | 51.02767248 | 132.1214445 |
| PMX109 | 402.9333333 | 483.9466667 | 512.375 | 2.1185923 | 149.3776638 | 208.7832167 |
| PMX111 | 589.9716667 | 417.425 | 525.77 | 196.7615302 | 4.422943025 | 49.29426361 |
| | | | | | mean | sd |
| | | | | Activated PBMC | 133.3694444 | 63.04364532 |
| | | | | only | | |
| | | | | Activated PBMC | 254.0545833 | 110.5967559 |
| | | | | plus HEK WT | | |
| | | | | Activated PBMC | 781.07125 | 211.8972647 |
| | | | | plus HEK OX40L | | |

TABLE 7
Normalised IFN-γ levels in supernatant from PBMC activated with CD3 and CD28 antibodies
in presence of HEK cells expressing OX40L and the indicated anti-OX40L antibody.

| | | mean | | | SD | | |
|---|---|---|---|---|---|---|---|
| | | Relative | Relative | Relative | Relative | Relative | Relative |
| | | IFNg at | IFNg at | IFNg at | IFNg at | IFNg at | IFNg at |
| | | 10 ug/ml | 1 ug/ml | 0.1 ug/ml | 10 ug/ml | 1 ug/ml | 0.1 ug/ml |
| Exp | Antibody | (%) | (%) | (%) | (%) | (%) | (%) |
| 1 | PMX26 | 36.85 | 101.03 | 103.12 | 1.79 | 54.53 | 18.89 |
| | PMX27 | 43.80 | 95.31 | 125.28 | 8.25 | 23.34 | 28.67 |
| | PMX28 | 113.49 | 130.14 | | 45.10 | 67.75 | |
| | PMX29 | 127.40 | 155.59 | | 10.12 | 66.98 | |
| | PMX30 | 106.73 | 117.71 | 177.53 | 21.10 | 10.47 | 30.97 |
| | PMX31 | 80.37 | 83.93 | 124.95 | 13.79 | 11.40 | 40.97 |
| | PMX32 | 45.18 | 108.38 | 132.34 | 30.42 | 61.31 | 96.57 |
| | PMX33 | 65.27 | 121.58 | 152.20 | 35.96 | 46.96 | 57.74 |
| | PMX34 | 66.26 | 176.63 | 244.13 | 50.45 | 121.99 | 158.91 |
| | PMX35 | 130.49 | 122.58 | 143.14 | 37.92 | 28.14 | 16.65 |
| | PMX36 | 57.13 | 153.42 | 188.90 | 44.78 | 99.78 | 56.22 |
| | PMX37 | 62.49 | 115.18 | 149.22 | 71.57 | 45.10 | 70.83 |
| | PMX38 | 61.75 | 141.31 | 144.35 | 51.36 | 41.25 | 67.84 |
| | PMX39 | 81.21 | 93.74 | 128.88 | 31.96 | 19.58 | 33.81 |
| | PMX40 | 109.44 | 100.22 | 112.24 | 3.63 | 7.75 | 13.46 |
| | PMX41 | 72.88 | 142.83 | 191.35 | 89.31 | 59.19 | 142.43 |
| | PMX42 | 80.10 | 109.86 | 111.79 | 7.59 | 10.79 | 15.82 |
| | PMX43 | 65.16 | 87.24 | 105.48 | 15.05 | 13.69 | 30.13 |
| | PMX44 | 124.28 | 167.78 | 206.60 | 82.80 | 99.25 | 151.24 |
| | PMX45 | 85.95 | 96.76 | 107.28 | 8.53 | 6.39 | 10.35 |
| | PMX46 | 69.78 | 118.83 | 118.19 | 28.43 | 46.06 | 60.83 |
| | PMX48 | 70.07 | 116.84 | 134.04 | 28.29 | 44.44 | 56.53 |
| | PMX49 | 62.87 | 133.00 | 162.14 | 41.64 | 69.22 | 94.01 |
| | PMX50 | 40.21 | 134.25 | 176.37 | 26.13 | 64.00 | 67.39 |
| | PMX51 | 28.20 | 102.48 | 145.08 | 39.93 | 46.32 | 7.16 |
| 2 | PMX25 | 57.57 | 66.01 | 88.02 | 30.77 | 20.04 | 37.50 |
| | PMX47 | 57.52 | 113.69 | 138.12 | 22.64 | 66.59 | 89.73 |
| 3 | PMX52 | 31.05 | 90.26 | 145.65 | 9.16 | 17.94 | 7.42 |
| | PMX53 | 45.43 | 82.00 | 114.88 | 13.60 | 23.52 | 10.31 |
| | PMX54 | 32.85 | 68.23 | 125.22 | 12.75 | 7.12 | 31.26 |
| | PMX55 | 16.32 | 83.74 | 112.55 | 15.08 | 7.18 | 11.29 |
| | PMX56 | 31.32 | 75.19 | 115.11 | 16.20 | 9.11 | 19.77 |
| | PMX57 | 22.05 | 67.80 | 112.13 | 6.45 | 10.96 | 11.88 |
| | PMX58 | 31.94 | 72.82 | 130.99 | 6.72 | 5.44 | 38.25 |
| | PMX59 | 31.13 | 92.69 | 107.60 | 8.27 | 27.67 | 35.76 |
| | PMX60 | 24.25 | 57.69 | 90.97 | 9.36 | 6.32 | 15.66 |
| | PMX61 | 26.85 | 65.32 | 104.43 | 21.50 | 13.67 | 20.71 |
| | PMX62 | 44.21 | 99.70 | 137.68 | 19.17 | 19.64 | 27.61 |
| | PMX63 | 11.16 | 74.51 | 106.12 | 1.19 | 11.77 | 34.82 |
| | PMX64 | 34.24 | 60.78 | 100.54 | 7.41 | 3.92 | 45.60 |
| | PMX65 | 53.05 | 80.77 | 112.67 | 7.54 | 5.90 | 32.27 |
| 4 | PMX82 | 23.92 | 78.29 | 95.21 | 36.30 | 13.02 | 4.38 |
| | PMX83 | 31.78 | 67.45 | 80.09 | 6.58 | 10.73 | 4.05 |
| | PMX84 | 33.95 | 68.78 | 94.48 | 8.50 | 17.90 | 14.75 |
| | PMX85 | 13.53 | 66.46 | 95.80 | 7.94 | 10.97 | 11.30 |
| 5 | PMX025 | 44.04 | 142.92 | 166.79 | 29.45 | 45.27 | 44.31 |
| | PMX033 | 42.66 | 93.82 | 136.38 | 11.89 | 47.58 | 30.54 |
| | PMX034 | 19.56 | 41.66 | 117.01 | 22.87 | 18.17 | 44.41 |
| | PMX036 | 32.54 | 51.50 | 82.57 | 1.25 | 15.66 | 38.46 |
| | PMX037 | 66.17 | 72.25 | 87.86 | 25.17 | 23.07 | 36.38 |
| | PMX041 | 66.44 | 112.72 | 97.86 | 31.77 | 47.44 | 8.97 |
| | PMX044 | 34.37 | 96.74 | 124.76 | 23.88 | 34.86 | 40.87 |
| | PMX046 | 63.97 | 123.33 | 193.55 | 32.38 | 38.58 | 27.56 |
| | PMX048 | 57.21 | 82.88 | 160.73 | 9.75 | 8.38 | 28.21 |
| | PMX049 | 59.93 | 84.15 | 129.02 | 22.55 | 12.52 | 19.77 |
| | PMX050 | 53.86 | 124.04 | 141.28 | 19.28 | 21.63 | 22.47 |
| | PMX051 | 15.44 | 63.72 | 151.08 | 9.96 | 11.59 | 4.91 |
| | PMX053 | 57.29 | 191.96 | 191.45 | 5.75 | 45.23 | 22.22 |
| | PMX063 | 26.41 | 117.80 | 159.56 | 5.83 | 15.36 | 41.09 |
| 6 | PMX044 | 41.22 | 44.04 | 87.18 | 21.14 | 14.22 | 16.98 |
| | PMX050 | 5.10 | 41.02 | 102.55 | 3.24 | 16.66 | 8.62 |
| | PMX051 | 9.65 | 13.35 | 89.97 | 19.12 | 24.57 | 3.50 |
| | PMX063 | −5.73 | −4.88 | 70.84 | 15.57 | 49.56 | 10.04 |
| | PMX097 | 12.00 | 15.52 | 85.77 | 44.58 | 13.20 | 3.66 |
| | PMX098 | 13.96 | 21.48 | 89.72 | 18.10 | 15.12 | 8.31 |
| | PMX154 | 12.69 | 7.20 | 101.80 | 41.61 | 18.90 | 25.82 |
| | PMX155 | −14.43 | 1.63 | 54.63 | 16.49 | 24.74 | 36.77 |

Experiment 8

| | mean | | | SD | | |
|---|---|---|---|---|---|---|
| | Relative IFNg | | Relative IFNg | Relative IFNg | | Relative IFNg |
| | at 10 ug/ml | Relative IFNg | at 0.1 ug/ml | at 10 ug/ml | Relative IFNg | at 0.1 ug/ml |
| Antibody | (%) | at 1 ug/ml (%) | (%) | (%) | at 1 ug/ml (%) | (%) |
| PMX155 | 37.23107119 | 144.7754983 | 159.9874572 | 29.48143486 | 53.68717611 | 21.9135431 |
| PMX156 | 36.9426653 | 169.274514 | 144.3941831 | 8.566680974 | 68.3673556 | 51.09172305 |
| PMX154 | 25.66193023 | 75.03353444 | 143.8173713 | 23.04242944 | 25.74891229 | 6.007934818 |
| PMX086 | 84.82384981 | 89.71320067 | 122.0785354 | 22.338678 | 52.0726459 | 6.842312076 |
| PMX087 | 62.12688698 | 118.5615321 | 153.6154291 | 30.77879239 | 9.146470622 | 63.91046008 |
| PMX088 | 74.18380165 | 67.02591588 | 122.3708125 | 33.76114995 | 21.39195581 | 29.67451998 |
| PMX089 | 43.93795983 | 83.6992604 | 89.56028748 | 21.93821524 | 9.387297612 | 31.07156125 |
| PMX099 | 56.27940917 | 60.04804184 | 43.26823877 | 14.37497664 | 20.83020591 | 37.06590208 |
| PMX100 | 58.30599284 | 49.16217121 | 52.65014043 | 29.50028368 | 22.82019568 | 11.61105291 |
| PMX101 | 52.99080779 | 40.26417205 | 61.35070737 | 36.88866577 | 15.85191983 | 19.3294834 |
| PMX092 | 50.98358022 | 61.25779809 | 48.13629791 | 12.42518384 | 22.50402658 | 31.87512432 |
| PMX093 | 3.905673854 | 27.60528267 | | 26.048774 | 1.961577197 | |
| PMX097 | 8.162080239 | 69.36993959 | | 3.858484509 | 20.80069186 | |
| PMX098 | 12.917874 | 22.94239818 | 43.4095383 | 13.36315606 | 2.207331316 | 39.98470385 |

Experiment 9

| | mean | | | SD | | |
|---|---|---|---|---|---|---|
| | Relative IFNg | | Relative IFNg | Relative IFNg | | Relative IFNg |
| | at 10 ug/ml | Relative IFNg | at 0.1 ug/ml | at 10 ug/ml | Relative IFNg | at 0.1 ug/ml |
| Antibody | (%) | at 1 ug/ml (%) | (%) | (%) | at 1 ug/ml (%) | (%) |
| PMX097 | 38.29939837 | 57.32463653 | 70.67951498 | 12.26987594 | 21.25298776 | 6.753109991 |
| PMX063 | 31.38780366 | 84.6014294 | 80.75959194 | 15.07872788 | 10.75722663 | 15.95598201 |
| PMX105 | 51.18427825 | 52.06436533 | 69.92177941 | 33.69696996 | 15.26705057 | 16.31093198 |
| PMX294 | 36.64638726 | 68.37677515 | 96.93699319 | 6.269384356 | 4.792497784 | 12.22764346 |
| PMX293 | 45.23799479 | 61.77570015 | 97.33020563 | 10.60888335 | 15.1152017 | 21.12089463 |
| PMX291 | 40.99788205 | 73.07254179 | 79.66686637 | 14.30575138 | 22.10364887 | 14.86231211 |
| PMX292 | 39.21998802 | 69.07206926 | 64.14594426 | 17.79164235 | 27.83180921 | 19.74510729 |

Experiment 10

| | mean | | | SD | | |
|---|---|---|---|---|---|---|
| | Relative IFNg | Relative IFNg | Relative IFNg | Relative IFNg | | Relative IFNg |
| | at 10 ug/ml | at 1 ug/ml | at 0.1 ug/ml | at 10 ug/ml | Relative IFNg | at 0.1 ug/ml |
| Antibody | (%) | (%) | (%) | (%) | at 1 ug/ml (%) | (%) |
| PMX105 | 9.326399485 | 26.14149091 | 80.09275049 | 5.509336216 | 19.82455021 | 11.41180542 |
| PMX108 | 43.31374253 | 61.93007411 | 85.5705791 | 3.57248725 | 7.878265059 | 20.39849575 |
| PMX109 | 41.61851823 | 54.12632982 | 58.51543743 | 0.3270937294 | 23.06271817 | 32.23446105 |
| PMX111 | 70.49574297 | 43.85591582 | 60.58351844 | 30.37840868 | 0.6828670758 | 7.610640576 |

TABLE 8
Functional affinity of anti OX40L antibodies for trimeric
canine OX40L determined by surface plasmon resonance (SPR).

| | | SPR Koff | SPR Koff |
|---|---|---|---|
| PMX number | SPR KD1 | (kd1) | (kd2) |
| PMX25 | 1.95E−07 | 1.29E−02 | 1.69E−03 |
| PMX26 | 9.03E−07 | 1.91E−03 | 1.31E−03 |
| PMX27 | 3.16E−07 | 1.61E−02 | 5.05E−04 |
| PMX28 | 1.70E−08 | 9.35E−03 | 6.27E−04 |
| PMX29 | 5.16E−08 | 2.94E−02 | 7.79E−04 |
| PMX30 | 1.14E−07 | 2.83E−02 | 9.24E−04 |
| PMX31 | 1.54E−07 | 1.24E−02 | 6.06E−04 |
| PMX32 | 7.20E−09 | 5.86E−03 | 1.71E−05 |
| PMX33 | 3.64E−08 | 1.03E−02 | 1.01E−03 |
| PMX34 | 7.23E−06 | 1.35E−01 | 2.52E−04 |
| PMX35 | 2.69E−07 | 3.11E−02 | 4.98E−04 |
| PMX36 | 2.16E−08 | 1.66E−03 | 5.22E−02 |
| PMX37 | 3.08E−07 | 1.83E−02 | 8.62E−04 |
| PMX38 | 1.00E−07 | 1.05E−02 | 7.96E−04 |
| PMX39 | 4.64E−08 | 1.28E−02 | 1.39E−03 |
| PMX40 | 1.26E−09 | 7.48E−04 | 4.36E−01 |
| PMX41 | 1.59E−06 | 1.34E−02 | 4.09E−04 |
| PMX42 | 2.74E−07 | 2.92E−02 | 5.60E−04 |
| PMX43 | 1.70E−08 | 2.49E−03 | 2.20E−03 |
| PMX44 | 8.23E−09 | 1.71E−04 | 5.98E−02 |
| PMX45 | 1.04E−08 | 4.84E−03 | 5.54E−04 |
| PMX46 | 4.04E−08 | 1.01E−03 | 2.02E−05 |
| PMX47 | 1.25E−08 | 4.51E−04 | 1.50E−05 |
| PMX48 | 1.22E−08 | 4.85E−03 | 2.10E+01 |
| PMX49 | 1.56E−08 | 2.11E−03 | 2.20E+01 |
| PMX50 | 1.31E−09 | 5.25E−04 | 1.61E−01 |
| PMX51 | 7.92E−09 | 1.19E−03 | 4.81E+00 |
| PMX52 | 1.20E−08 | 2.76E−03 | 7.25E−02 |
| PMX53 | 3.40E−09 | 5.87E−04 | 5.65E−01 |
| PMX54 | 5.76E−09 | 3.45E−02 | 8.48E−02 |
| PMX55 | 5.43E−09 | 3.66E−03 | 4.01E−01 |
| PMX56 | 6.10E−08 | 1.48E−02 | 6.24E−04 |
| PMX57 | 1.29E−09 | 4.72E−03 | 2.20E−01 |
| PMX58 | 8.46E−09 | 1.44E−02 | 3.23E−01 |
| PMX59 | 1.00E−08 | 7.37E−03 | 7.37E−03 |
| PMX60 | 7.09E−08 | 9.33E−03 | 9.25E−04 |
| PMX61 | 8.96E−08 | 7.46E−03 | 7.29E−04 |
| PMX62 | N/D | N/D | N/D |
| PMX63 | 2.71E−10 | 9.05E−05 | 5.21E+00 |
| PMX64 | 1.45E−07 | 3.52E−02 | 9.21E−04 |
| PMX65 | 1.07E−07 | 1.25E−02 | 4.12E−01 |
| PMX097 | 4.92E−10 | 7.16E−05 | 7.44E−01 |
| PMX098 | 1.41E−08 | 1.47E−03 | 1.03E+00 |
| PMX154 | 1.56E−08 | 3.84E−03 | 6.94E+00 |
| PMX155 | 9.06E−08 | 3.20E−02 | 2.94E−04 |
| PMX156 | 1.98E−06 | 1.02E−01 | 2.38E−04 |
| PMX105 | 1.76E−08 | 1.74E−03 | 1.23E+00 |
| PMX294 | 1.44E−08 | 1.55E−03 | 2.69E+00 |
| PMX293 | 1.00E−09 | 3.87E−04 | 1.45E+02 |
| PMX104 | 1.76E−08 | 1.74E−03 | 1.23E+00 |

TABLE 9
Stability determination of OX40L antibodies.

| | | | Initial | % monomer |
|---|---|---|---|---|
| Antibody | Tm1 | Tagg266 | diameter | (SEC) |
| PMX25 | | | | 91.01 |
| PMX26 | | 67.40 | 9.85 | 97.37 |
| PMX27 | | 68.00 | 9.79 | 97.90 |
| PMX28 | 60.10 | 63.10 | 9.99 | 97.74 |
| PMX29 | 61.50 | 62.50 | 10.07 | 95.28 |
| PMX30 | 60.90 | 59.00 | 12.60 | 93.17 |
| PMX31 | 58.9 | 57.4 | 10.84 | 94.07 |
| PMX32 | 60.2 | 61.7 | 10.13 | 97.11 |
| PMX33 | 62.50 | 62.40 | 10.52 | 32.73 |
| PMX34 | 57.70 | 58.70 | 10.76 | 97.07 |
| PMX35 | | 51.1 | 14.20 | 94.89 |
| PMX36 | 61 | 62.30 | 10.13 | 95.33 |
| PMX37 | | 55.90 | 11.00 | 97.87 |
| PMX38 | 58.2 | 59.1 | 10.41 | 89.30 |
| PMX39 | 59.6 | 59.9 | 12.09 | |
| PMX40 | | 54.5 | 24.88 | 97.75 |
| PMX41 | 60.00 | 60.00 | 11.70 | 94.88 |
| PMX42 | 62.9 | 64.1 | | 96.90 |
| PMX43 | 66.90 | 69.10 | 11.19 | 96.06 |
| PMX44 | 62.30 | 62.90 | 12.39 | 88.77 |
| PMX45 | 57.8 | 58.6 | 11.88 | 93.19 |
| PMX46 | 62.30 | 62.90 | 12.39 | 95.25 |
| PMX47 | | | | 75.43 |
| PMX48 | 64.6 | 61.4 | 9.97 | 97.22 |
| PMX49 | 64.6 | 61.4 | 9.97 | 97.32 |
| PMX50 | 64.9 | 65.9 | 42.33 | 89.26 |
| PMX51 | 63 | 62.5 | 10.24 | 96.68 |
| PMX53 | 61.2 | 58.4 | 9.72 | 93.21 |
| PMX63 | 63 | 61 | 9.74 | 94.58 |
| PMX097 | 65.8 | 66.7 | 10.1 | 99.28 |
| PMX098 | 62.2 | 60.6 | 14.66 | 97.5 |
| PMX154 | 67.2 | 68.8 | 10.04 | 99.19 |
| PMX155 | 62.5 | 62.1 | 10.6 | 99.43 |
| PMX156 | 63.4 | 62.7 | 10.33 | 99.63 |

TABLE 10
Long term stability of PMX097 and PMX154 at 4° C.

| | After purification | 4 months at 4 C. |
|---|---|---|
| | 5 mg/ml | 5 mg/ml |
| PMX097 | | |
| Tm | 65.8 ± 0.17 | 69.4 ± 0.85 |
| Tagg 266 nm | 66.7 ± 0.23 | 64.7 ± 0.94 |
| Z AVG diam | 10.1 | 9.92 ± 0.08 |
| PDI | 0.05 | 0.061 ± 0.018 |
| PMX154 | | |
| Tm | 67.2 ± 0.21 | 66.71 ± 0.35 (N = 2) |
| Tagg 266 nm | 68.8 ± 0.23 | 61.4 ± 3.5 |
| Z AVG diam | 10.04 | 10.23 ± 0.20 |
| PDI | 0.03 | 0.060 ± 0.016 |

TABLE 11
Treatment regime of dogs in the single dose antigen recall dog study biopsies

| | Test | 1^{st} | 2^{nd} | Skin | 3^{rd} |
|---|---|---|---|---|---|
| | substance | immunisation | immunisation | biopsies | immunisation |
| Group | (Day 0) | (day 1) | (day 21) | (day 23) | (day 63) |
| 1 | Vehicle control | 100 μg | 10 μg KLH | 2 biopsies per | 100 μg KLH |
| 2 | PMX154 at 3.0 | KLH + 100 μg | intradermal | dog, one in KLH | intramuscular |
| | mg/kg | alhydrogel | | injected area, | |
| 3 | PMX154 at 0.5 | intramuscular | | one in control | |
| | mg/kg | injection | | area | |
| 4 | PMX097 at 3.0 | | | | |
| | mg/kg | | | | |
| 5 | PMX097 at 0.5 | | | | |
| | mg/kg | | | | |

TABLE 12
Histological findings in non-injected skin areas from dogs in the single dose antigen recall study.

| | Group 1 | | Group 2 | | Group 3 | |
|---|---|---|---|---|---|---|
| | Non injected area | | Non injected area | | Non injected area | |
| | N of dogs | | N of dogs | | N of dogs | |
| | with | Mean | with | Mean | with | Mean |
| | abnormalities | score | abnormalities | score | abnormalities | score |
| Hyperkeratosis | 0 | 0 | 0 | 0 | 0 | 0 |
| Infiltrate, lymphocytic, dermal | 0 | 0 | 0 | 0 | 0 | 0 |
| Infiltrate, histiocytic/macrophagic, | 0 | 0 | 0 | 0 | 0 | 0 |
| dermal | | | | | | |
| Infiltrate, neutrophilic, dermal | 0 | 0 | 0 | 0 | 0 | 0 |
| Infiltrate, lymphocytic, | 0 | 0 | 0 | 0 | 0 | 0 |
| subcutaneous/muscular | | | | | | |
| Infiltrate, histiocytic/macrophagic, | 0 | 0 | 0 | 0 | 0 | 0 |
| subcutaneous/muscular | | | | | | |
| Infiltrate, neutrophilic, | 0 | 0 | 0 | 0 | 0 | 0 |
| subcutaneous/muscular | | | | | | |
| Fibroplasia, subcutaneous | 0 | 0 | 0 | 0 | 0 | 0 |
| Hemorrhage, subcutaneous | 0 | 0 | 0 | 0 | 0 | 0 |
| Myofiber loss | — | — | — | — | 0 | 0 |
| Myofiber degeneration | — | — | — | — | 0 | 0 |
| Artifact | 4 | 1 | 4 | 1 | 4 | 1 |
| Sum score | 0 | 0 | 0 | 0 | 0 | 0 |
| No Significant findings | 4 | | 4 | | 4 | |

| | |
|---|---|
| Group 4 | Group 5 |
| Non injected area | Non injected area |

| | N of dogs | | N of dogs | |
|---|---|---|---|---|
| | with | Mean | with | Mean |
| | abnormalities | score | abnormalities | score |
| Hyperkeratosis | 0 | 0 | 0 | 0 |
| Infiltrate, lymphocytic, dermal | 0 | 0 | 0 | 0 |
| Infiltrate, histiocytic/macrophagic, | 0 | 0 | 0 | 0 |
| dermal | | | | |
| Infiltrate, neutrophilic, dermal | 0 | 0 | 0 | 0 |
| Infiltrate, lymphocytic, | 0 | 0 | 0 | 0 |
| subcutaneous/muscular | | | | |
| Infiltrate, histiocytic/macrophagic, | 0 | 0 | 0 | 0 |
| subcutaneous/muscular | | | | |
| Infiltrate, neutrophilic, | 0 | 0 | 0 | 0 |
| subcutaneous/muscular | | | | |
| Fibroplasia, subcutaneous | 0 | 0 | 0 | 0 |
| Hemorrhage, subcutaneous | 0 | 0 | 0 | 0 |
| Myofiber loss | 0 | 0 | — | — |
| Myofiber degeneration | 0 | 0 | — | — |
| Artifact | 4 | 1 | 4 | 1 |
| Sum score | 0 | 0 | 0 | 0 |
| No Significant findings | 4 | | 4 | |

TABLE 13
Histological findings in KLH-injected skin areas from dogs in the single dose antigen recall study.

| | Group 1 | | Group 2 | | Group 3 | |
|---|---|---|---|---|---|---|
| | KLH injected area | | KLH injected area | | KLH injected area | |
| | N of dogs | | N of dogs | | N of dogs | |
| | with | Mean | with | Mean | with | Mean |
| | abnormalities | score | abnormalities | score | abnormalities | score |
| Hyperkeratosis | 1 | 0.3 | 0 | 0 | 0 | 0 |
| Infiltrate, lymphocytic, dermal | 4 | 1.3 | 3 | 1.3 | 0 | 0 |
| Infiltrate, histiocytic/macrophagic, | 2 | 0.5 | 0 | 0 | 0 | 0 |
| dermal | | | | | | |
| Infiltrate, neutrophilic, dermal | 2 | 0.5 | 0 | 0 | 0 | 0 |
| Infiltrate, lymphocytic, | 3 | 2 | 4 | 1.3 | 0 | 0 |
| subcutaneous/muscular | | | | | | |
| Infiltrate, histiocytic/macrophagic, | 3 | 2.7 | 4 | 1 | 0 | 0 |
| subcutaneous/muscular | | | | | | |
| Infiltrate, neutrophilic, | 3 | 2.3 | 4 | 1.3 | 0 | 0 |
| subcutaneous/muscular | | | | | | |
| Fibroplasia, subcutaneous | 3 | 1.3 | 1 | 0.3 | 0 | 0 |
| Hemorrhage, subcutaneous | 3 | 1 | 3 | 0.8 | 1 | 0.5 |
| Myofiber loss | 1 | 1 | 1 | 1 | — | — |
| Myofiber degeneration | 0 | 0 | 1 | 1 | — | — |
| Artifact | 3 | 0.8 | 4 | 1 | 4 | 1 |
| Sum score | 25 | 12.8 | 21 | 7.8 | 1 | 0.5 |
| N. of dogs with No Significant | 0 | | 0 | | 3 | |
| findings | | | | | | |

| | |
|---|---|
| Group 4 | Group 5 |
| KLH injected area | KLH injected area |

| | N of dogs | | N of dogs | |
|---|---|---|---|---|
| | with | Mean | with | Mean |
| | abnormalities | score | abnormalities | score |
| Hyperkeratosis | 0 | 0 | 0 | 0 |
| Infiltrate, lymphocytic, dermal | 3 | 1.8 | 2 | 1 |
| Infiltrate, histiocytic/macrophagic, | 2 | 1 | 2 | 1 |
| dermal | | | | |
| Infiltrate, neutrophilic, dermal | 2 | 0.8 | 2 | 1 |
| Infiltrate, lymphocytic, | 2 | 1.3 | 3 | 1.7 |
| subcutaneous/muscular | | | | |
| Infiltrate, histiocytic/macrophagic, | 2 | 1 | 3 | 2 |
| subcutaneous/muscular | | | | |
| Infiltrate, neutrophilic, | 2 | 1 | 3 | 2 |
| subcutaneous/muscular | | | | |
| Fibroplasia, subcutaneous | 1 | 0.7 | 0 | 0 |
| Hemorrhage, subcutaneous | 1 | 0.7 | 1 | 0.3 |
| Myofiber loss | 0 | 0 | 0 | 0 |
| Myofiber degeneration | 0 | 0 | 0 | 0 |
| Artifact | 4 | 1 | 0 | 0 |
| Sum score | 15 | 8.2 | 16 | 9 |
| N. of dogs with No Significant | 1 | | 0 | |
| findings | | | | |

Sequences Used in Experimental

SEQ ID NO. 807
Monomeric OX40L probe amino acid sequence
NGGPSVFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQVNLTCLV
KGFYPSDIAVEWESNGQPENNYKTTPPVLDSDGSFFLNSTLTVDKSRWQQGNVFSCSVMHEALHNH
YTQKSLSLSHHHHHHENLYFQGQVPPQYPPIQSIRVQFTRCENEKGCIITSPSKDETMKVQDNSIIINC
DGFYLISLKGYFSEELSLSLYYRKGRGPLFSLSKVTSVDSIGVAYLAFKDKVYFNVTTHSTSYKDIQVN
GGELILIHQNPGGFCAY
SEQ ID NO. 808
Trimeric OX40L probe (hIgG1 Fc fusion) DNA sequence
gacaaaactcacacatgcccaccgtgcccagcacctgaactcctggggggaccgtcagtcttcctcttccccccaaaacccaaggacaccct
catgatctcccggacccctgaggtcacatgcgtggtggtggacgtgagccacgaagaccctgaggtcaagttcaactggtacgtggacggcgt
ggaggtgcataatgccaagacaaagccgcgggaggagcagtacaacagcacgtaccgtgtggtcagcgtcctcaccgtcctgcaccagga
ctggctgaatggcaaggagtacaagtgcaaggtctccaacaaagccctcccagcccccatcgagaaaaccatctccaaagccaaagggc
agccccgagaaccacaggtgtacaccctgcccccatcccgggatgagctgaccaagaaccaggtcagcctgacctgcctggtcaaaggctt
ctatcccagcgacatcgccgtggagtgggagagcaatgggcagccggagaacaactacaagaccacgcctcccgtgctggactccgacg
gctccttcttcctctacagcaagctcaccgtggacaagagcaggtggcagcaggggaacgtcttctcatgctccgtgatgcatgaggctctgca
caaccactacacacagaagagcctctccctgtctccgggtaaaAGGAGCCCCCAGCCCCAGCCCAAGCCCCAGCCC
AAGCCCGAGCCCGAGGGCAGCCTGCACGCCTGCGGCTGCGCCGCCGCCCCCGACATCAAGGA
CCTGCTGAGCAGGCTGGAGGAGCTGGAGGGCCTGGTGAGCAGCCTGAGGGAGCAGGGCACCG
GCGGCGGCAGCGGCGGCAGGGGCCTGCAGCAGGTGCCGCCTCAGTATCCTCCAATTCAAAGTA
TCAGAGTACAATTTACCAGGTGTGAGAATGAGAAAGGTTGCATCATCACATCCCCAAGCAAGGAT
GAAACTATGAAGGTGCAAGACAACTCAATCATCATTAACTGTGATGGGTTTTATCTCATCTCCCTG
AAGGGTTACTTCTCTGAGGAGCTCAGCCTCAGCCTTTATTACCGAAAGGGTCGGGGACCCCTCTT
CTCTCTGAGCAAGGTCACATCTGTTGACTCCATTGGAGTGGCCTATCTGGCTTTCAAGGACAAAG
TCTACTTTAATGTGACCACTCACAGTACCTCCTACAAAGACATCCAGGTGAATGGTGGGGAATTG
ATTCTCATTCATCAAAATCCTGGTGGCTTCTGTGCCTAC
SEQ ID NO. 809
Trimeric OX40L probe (hIgG1 Fc fusion) amino acid sequence
DKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNV
FSCSVMHEALHNHYTQKSLSLSPGKRSPQPQPKPQPKPEPEGSLHACGCAAAPDIKDLLSRLEELEG
LVSSLREQGTGGGSGGRGLQQVPPQYPPIQSIRVQFTRCENEKGCIITSPSKDETMKVQDNSIIINCDG
FYLISLKGYFSEELSLSLYYRKGRGPLFSLSKVTSVDSIGVAYLAFKDKVYFNVTTHSTSYKDIQVNGGE
LILIHQNPGGFCAY
SEQ ID NO. 810
Trimeric OX40L probe (HIS tag) DNA sequence
CACCATCACcatcaccatGGCCCCGGCCAGGTGCAGCTGCACGCCTGCGGCTGCGCCGCCGCCCC
CGACATCAAGGACCTGCTGAGCAGGCTGGAGGAGCTGGAGGGCCTGGTGAGCAGCCTGAGGGA
GCAGGGCACCGGCGGCGGCAGCGGCGGCAGGGGCCTGCAGCAGGTGCCGCCTCAGTATCCTC
CAATTCAAAGTATCAGAGTACAATTTACCAGGTGTGAGAATGAGAAAGGTTGCATCATCACATCCC
CAAGCAAGGATGAAACTATGAAGGTGCAAGACAACTCAATCATCATTAACTGTGATGGGTTTTATC
TCATCTCCCTGAAGGGTTACTTCTCTGAGGAGCTCAGCCTCAGCCTTTATTACCGAAAGGGTCGG
GGACCCCTCTTCTCTCTGAGCAAGGTCACATCTGTTGACTCCATTGGAGTGGCCTATCTGGCTTT
CAAGGACAAAGTCTACTTTAATGTGACCACTCACAGTACCTCCTACAAAGACATCCAGGTGAATG
GTGGGGAATTGATTCTCATTCATCAAAATCCTGGTGGCTTCTGTGCCTAC
SEQ ID NO. 811
Trimeric OX40L probe (HIS tag) amino acid sequence
HHHHHHGPGQVQLHACGCAAAPDIKDLLSRLEELEGLVSSLREQGTGGGSGGRGLQQVPPQYPPIQ
SIRVQFTRCENEKGCIITSPSKDETMKVQDNSIIINCDGFYLISLKGYFSEELSLSLYYRKGRGPLFSLSK
VTSVDSIGVAYLAFKDKVYFNVTTHSTSYKDIQVNGGELILIHQNPGGFCAY

1. (canceled)

2. An antibody or fragment thereof that specifically binds to companion animal OX40L, wherein the antibody is selected from the group consisting of:
an antibody comprising a HC CDR1 comprising SEQ ID No: 575 or a sequence with at least 80% sequence identity thereto, a HC CDR2 comprising SEQ ID No: 576 or a sequence with at least 80% sequence identity thereto, a HC CDR3 comprising SEQ ID No: 577 or a sequence with at least 80% sequence identity thereto, a LC CDR1 comprising SEQ ID No: 578 or a sequence with at least 80% sequence identity thereto, a LC CDR2 comprising SEQ ID No: 579 or a sequence with at least 80% sequence identity thereto and a LC CDR3 comprising SEQ ID No: 580 or a sequence with at least 80% sequence identity thereto;
an antibody comprising a HC CDR1 comprising SEQ ID No: 741 or a sequence with at least 80%, sequence identity thereto, a HC CDR2 comprising SEQ ID No: 742 or a sequence with at least 80%, sequence identity thereto, a HC CDR3 comprising SEQ ID No: 743 or a sequence with at least 80% sequence identity thereto, a LC CDR1 comprising SEQ ID No: 744 or a sequence with at least 80% sequence identity thereto, a LC CDR2 comprising SEQ ID No: 745 or a sequence with at least 80% sequence identity thereto and a LC CDR3 comprising SEQ ID No: 746 or a sequence with at least 80% sequence identity thereto; or
an antibody comprising a HC CDR1 comprising SEQ ID No: 275 or a sequence with at least 80% sequence identity thereto, a HC CDR2 comprising SEQ ID No: 276 or a sequence with at least 80% sequence identity thereto, a HC CDR3 comprising SEQ ID No: 277 or a sequence with at least 80% sequence identity thereto, a LC CDR1 comprising SEQ ID No: 278 or a sequence with at least 80% sequence identity thereto, a LC CDR2 comprising SEQ ID No: 279 or a sequence with at least 80% sequence identity thereto and a LC CDR3 comprising SEQ ID No: 280 or a sequence with at least 80% sequence identity thereto;
an antibody comprising a HC CDR1 comprising SEQ ID No: 395 or a sequence with at least 80% sequence identity thereto, a HC CDR2 comprising SEQ ID No: 396 or a sequence with at least 80% sequence identity thereto, a HC CDR3 comprising SEQ ID No: 397 or a sequence with at least 80% sequence identity thereto, a LC CDR1 comprising SEQ ID No: 398 or a sequence with at least 80% sequence identity thereto, a LC CDR2 comprising SEQ ID No: 399 or a sequence with at least 80% sequence identity thereto and a LC CDR3 comprising SEQ ID No: 400 or a sequence with at least 80% sequence identity thereto;
an antibody comprising a HC CDR1 comprising SEQ ID No: 15 or a sequence with at least 80% sequence identity thereto, a HC CDR2 comprising SEQ ID No: 16 or a sequence with at least 80% sequence identity thereto, a HC CDR3 comprising SEQ ID No: 17 or a sequence with at least 80% sequence identity thereto, a LC CDR1 comprising SEQ ID No: 18 or a sequence with at least 80% sequence identity thereto, a LC CDR2 comprising SEQ ID No: 19 or a sequence with at least 80% sequence identity thereto and a LC CDR3 comprising SEQ ID No: 20 or a sequence with at least 80% sequence identity thereto;
an antibody comprising a HC CDR1 comprising SEQ ID No: 25 or a sequence with at least 80% sequence identity thereto, a HC CDR2 comprising SEQ ID No: 26 or a sequence with at least 80% sequence identity thereto, a HC CDR3 comprising SEQ ID No: 27 or a sequence with at least 80% sequence identity thereto, a LC CDR1 comprising SEQ ID No: 28 or a sequence with at least 80% sequence identity thereto, a LC CDR2 comprising SEQ ID No: 29 or a sequence with at least 80% sequence identity thereto and a LC CDR3 comprising SEQ ID No: 30 or a sequence with at least 80% sequence identity thereto;
an antibody comprising a HC CDR1 comprising SEQ ID No: 35 or a sequence with at least 80% sequence identity thereto, a HC CDR2 comprising SEQ ID No: 36 or a sequence with at least 80% sequence identity thereto, a HC CDR3 comprising SEQ ID No: 37 or a sequence with at least 80% sequence identity thereto, a LC CDR1 comprising SEQ ID No: 38 or a sequence with at least 80% sequence identity thereto, a LC CDR2 comprising SEQ ID No: 39 or a sequence with at least 80% sequence identity thereto and a LC CDR3 comprising SEQ ID No: 40 or a sequence with at least 80% sequence identity thereto;
an antibody comprising a HC CDR1 comprising SEQ ID No: 45 or a sequence with at least 80% sequence identity thereto, a HC CDR2 comprising SEQ ID No: 46 or a sequence with at least 80% sequence identity thereto, a HC CDR3 comprising SEQ ID No: 47 or a sequence with at least 80% sequence identity thereto, a LC CDR1 comprising SEQ ID No: 48 or a sequence with at least 80% sequence identity thereto, a LC CDR2 comprising SEQ ID No: 49 or a sequence with at least 80% sequence identity thereto and a LC CDR3 comprising SEQ ID No: 50 or a sequence with at least 80% sequence identity thereto;
an antibody comprising a HC CDR1 comprising SEQ ID No: 55 or a sequence with at least 80% sequence identity thereto, a HC CDR2 comprising SEQ ID No: 56 or a sequence with at least 80% sequence identity thereto, a HC CDR3 comprising SEQ ID No: 57, or a sequence with at least 80% sequence identity thereto, a LC CDR1 comprising SEQ ID No: 58 or a sequence with at least 80% sequence identity thereto, a LC CDR2 comprising SEQ ID No: 59 or a sequence with at least 80% sequence identity thereto and a LC CDR3 comprising SEQ ID No: 60 or a sequence with at least 80% sequence identity thereto;
an antibody comprising a HC CDR1 comprising SEQ ID No: 65 or a sequence with at least 80% sequence identity thereto, a HC CDR2 comprising SEQ ID No: 66 or a sequence with at least 80% sequence identity thereto, a HC CDR3 comprising SEQ ID No: 67 or a sequence with at least 80% sequence identity thereto, a LC CDR1 comprising SEQ ID No: 68 or a sequence with at least 80% sequence identity thereto, a LC CDR2 comprising SEQ ID No: 69 or a sequence with at least 80% sequence identity thereto and a LC CDR3 comprising SEQ ID No: 70 or a sequence with at least 80% sequence identity thereto;
an antibody comprising a HC CDR1 comprising SEQ ID No: 75 or a sequence with at least 80% sequence identity thereto, a HC CDR2 comprising SEQ ID No: 76 or a sequence with at least 80% sequence identity thereto, a HC CDR3 comprising SEQ ID No: 77 or a sequence with at least 80% sequence identity thereto, a LC CDR1 comprising SEQ ID No: 78 or a sequence with at least 80% sequence identity thereto, a LC CDR2 comprising SEQ ID No: 79 or a sequence with at least 80% sequence identity thereto and a LC CDR3 comprising SEQ ID No: 80 or a sequence with at least 80% sequence identity thereto;
an antibody comprising a HC CDR1 comprising SEQ ID No: 85 or a sequence with at least 80% sequence identity thereto, a HC CDR2 comprising SEQ ID No: 86 or a sequence with at least 80% sequence identity thereto, a HC CDR3 comprising SEQ ID No: 87 or a sequence with at least 80% sequence identity thereto, a LC CDR1 comprising SEQ ID No: 88 or a sequence with at least 80% sequence identity thereto, a LC CDR2 comprising SEQ ID No: 89 or a sequence with at least 80% sequence identity thereto and a LC CDR3 comprising SEQ ID No: 90 or a sequence with at least 80% sequence identity thereto;
an antibody comprising a HC CDR1 comprising SEQ ID No: 95 or a sequence with at least 80% sequence identity thereto, a HC CDR2 comprising SEQ ID No: 96 or a sequence with at least 80% sequence identity thereto, a HC CDR3 comprising SEQ ID No: 97 or a sequence with at least 80% sequence identity thereto, a LC CDR1 comprising SEQ ID No: 98 or a sequence with at least 80% sequence identity thereto, a LC CDR2 comprising SEQ ID No: 99 or a sequence with at least 80% sequence identity thereto and a LC CDR3 comprising SEQ ID No: 100 or a sequence with at least 80% sequence identity thereto;
an antibody comprising a HC CDR1 comprising SEQ ID No: 105 or a sequence with at least 80% sequence identity thereto, a HC CDR2 comprising SEQ ID No: 106 or a sequence with at least 80% sequence identity thereto, a HC CDR3 comprising SEQ ID No: 107 or a sequence with at least 80% sequence identity thereto, a LC CDR1 comprising SEQ ID No: 108 or a sequence with at least 80% sequence identity thereto, a LC CDR2 comprising SEQ ID No: 109 or a sequence with at least 80% sequence identity thereto and a LC CDR3 comprising SEQ ID No: 110 or a sequence with at least 80% sequence identity thereto;
an antibody comprising a HC CDR1 comprising SEQ ID No: 115 or a sequence with at least 80% sequence identity thereto, a HC CDR2 comprising SEQ ID No: 116 or a sequence with at least 80% sequence identity thereto, a HC CDR3 comprising SEQ ID No: 117 or a sequence with at least 80% sequence identity thereto, a LC CDR1 comprising SEQ ID No: 118 or a sequence with at least 80% sequence identity thereto, a LC CDR2 comprising SEQ ID No: 119 or a sequence with at least 80% sequence identity thereto and a LC CDR3 comprising SEQ ID No: 120 or a sequence with at least 80% sequence identity thereto;
an antibody comprising a HC CDR1 comprising SEQ ID No: 125 or a sequence with at least 80% sequence identity thereto, a HC CDR2 comprising SEQ ID No: 126 or a sequence with at least 80% sequence identity thereto, a HC CDR3 comprising SEQ ID No: 127 or a sequence with at least 80% sequence identity thereto, a LC CDR1 comprising SEQ ID No: 128 or a sequence with at least 80% sequence identity thereto, a LC CDR2 comprising SEQ ID No: 129 or a sequence with at least 80% sequence identity thereto and a LC CDR3 comprising SEQ ID No: 130 or a sequence with at least 80% sequence identity thereto; or
an antibody comprising a HC CDR1 comprising SEQ ID No: 135 or a sequence with at least 80% sequence identity thereto, a HC CDR2 comprising SEQ ID No: 136 or a sequence with at least 80% sequence identity thereto, a HC CDR3 comprising SEQ ID No: 137 or a sequence with at least 80% sequence identity thereto, a LC CDR1 comprising SEQ ID No: 138 or a sequence with at least 80% sequence identity thereto, a LC CDR2 comprising SEQ ID No: 139 or a sequence with at least 80% sequence identity thereto and a LC CDR3 comprising SEQ ID No: 140 or a sequence with at least 80% sequence identity thereto.
an antibody comprising a HC CDR1 comprising SEQ ID No: 145 or a sequence with at least 80% sequence identity thereto, a HC CDR2 comprising SEQ ID No: 146 or a sequence with at least 80% sequence identity thereto, a HC CDR3 comprising SEQ ID No: 147 or a sequence with at least 80% sequence identity thereto, a LC CDR1 comprising SEQ ID No: 148 or a sequence with at least 80% sequence identity thereto, a LC CDR2 comprising SEQ ID No: 149 or a sequence with at least 80% sequence identity thereto and a LC CDR3 comprising SEQ ID No: 150 or a sequence with at least 80% sequence identity thereto;
an antibody comprising a HC CDR1 comprising SEQ ID No: 155 or a sequence with at least 80% sequence identity thereto, a HC CDR2 comprising SEQ ID No: 156 or a sequence with at least 80% sequence identity thereto, a HC CDR3 comprising SEQ ID No: 157 or a sequence with at least 80% sequence identity thereto, a LC CDR1 comprising SEQ ID No: 158 or a sequence with at least 80% sequence identity thereto, a LC CDR2 comprising SEQ ID No: 159 or a sequence with at least 80% sequence identity thereto and a LC CDR3 comprising SEQ ID No: 160 or a sequence with at least 80% sequence identity thereto;
an antibody comprising a HC CDR1 comprising SEQ ID No: 165 or a sequence with at least 80% sequence identity thereto, a HC CDR2 comprising SEQ ID No: 166 or a sequence with at least 80% sequence identity thereto, a HC CDR3 comprising SEQ ID No: 167 or a sequence with at least 80% sequence identity thereto, a LC CDR1 comprising SEQ ID No: 168 or a sequence with at least 80% sequence identity thereto, a LC CDR2 comprising SEQ ID No: 169 or a sequence with at least 80% sequence identity thereto and a LC CDR3 comprising SEQ ID No: 170 or a sequence with at least 80% sequence identity thereto;
an antibody comprising a HC CDR1 comprising SEQ ID No: 175 or a sequence with at least 80% sequence identity thereto, a HC CDR2 comprising SEQ ID No: 176 or a sequence with at least 80% sequence identity thereto, a HC CDR3 comprising SEQ ID No: 177 or a sequence with at least 80% sequence identity thereto, a LC CDR1 comprising SEQ ID No: 178 or a sequence with at least 80% sequence identity thereto, a LC CDR2 comprising SEQ ID No: 179 or a sequence with at least 80% sequence identity thereto and a LC CDR3 comprising SEQ ID No: 180 or a sequence with at least 80% sequence identity thereto;
an antibody comprising a HC CDR1 comprising SEQ ID No: 185 or a sequence with at least 80% sequence identity thereto, a HC CDR2 comprising SEQ ID No: 186 or a sequence with at least 80% sequence identity thereto, a HC CDR3 comprising SEQ ID No: 187 or a sequence with at least 80% sequence identity thereto, a LC CDR1 comprising SEQ ID No: 188 or a sequence with at least 80% sequence identity thereto, a LC CDR2 comprising SEQ ID No: 189 or a sequence with at least 80% sequence identity thereto and a LC CDR3 comprising SEQ ID No: 190 or a sequence with at least 80% sequence identity thereto;
an antibody comprising a HC CDR1 comprising SEQ ID No: 195 or a sequence with at least 80% sequence identity thereto, a HC CDR2 comprising SEQ ID No: 196 or a sequence with at least 80% sequence identity thereto, a HC CDR3 comprising SEQ ID No: 197 or a sequence with at least 80% sequence identity thereto, a LC CDR1 comprising SEQ ID No: 198 or a sequence with at least 80% sequence identity thereto, a LC CDR2 comprising SEQ ID No: 199 or a sequence with at least 80% sequence identity thereto and a LC CDR3 comprising SEQ ID No: 200 or a sequence with at least 80% sequence identity thereto;
an antibody comprising a HC CDR1 comprising SEQ ID No: 205 or a sequence with at least 80% sequence identity thereto, a HC CDR2 comprising SEQ ID No: 206 or a sequence with at least 80% sequence identity thereto, a HC CDR3 comprising SEQ ID No: 207 or a sequence with at least 80% sequence identity thereto, a LC CDR1 comprising SEQ ID No: 208 or a sequence with at least 80% sequence identity thereto, a LC CDR2 comprising SEQ ID No: 209 or a sequence with at least 80% sequence identity thereto and a LC CDR3 comprising SEQ ID No: 210 or a sequence with at least 80% sequence identity thereto;
an antibody comprising a HC CDR1 comprising SEQ ID No: 215 or a sequence with at least 80% sequence identity thereto, a HC CDR2 comprising SEQ ID No: 216 or a sequence with at least 80% sequence identity thereto, a HC CDR3 comprising SEQ ID No: 217 or a sequence with at least 80% sequence identity thereto, a LC CDR1 comprising SEQ ID No: 218 or a sequence with at least 80% sequence identity thereto, a LC CDR2 comprising SEQ ID No: 219 or a sequence with at least 80% sequence identity thereto and a LC CDR3 comprising SEQ ID No: 220 or a sequence with at least 80% sequence identity thereto;
an antibody comprising a HC CDR1 comprising SEQ ID No: 225 or a sequence with at least 80% sequence identity thereto, a HC CDR2 comprising SEQ ID No: 226 or a sequence with at least 80% sequence identity thereto, a HC CDR3 comprising SEQ ID No: 227 or a sequence with at least 80% sequence identity thereto, a LC CDR1 comprising SEQ ID No: 228 or a sequence with at least 80% sequence identity thereto, a LC CDR2 comprising SEQ ID No: 229 or a sequence with at least 80% sequence identity thereto and a LC CDR3 comprising SEQ ID No: 230 or a sequence with at least 80% sequence identity thereto;
an antibody comprising a HC CDR1 comprising SEQ ID No: 235 or a sequence with at least 80% sequence identity thereto, a HC CDR2 comprising SEQ ID No: 236 or a sequence with at least 80% sequence identity thereto, a HC CDR3 comprising SEQ ID No: 237 or a sequence with at least 80% sequence identity thereto, a LC CDR1 comprising SEQ ID No: 238 or a sequence with at least 80% sequence identity thereto, a LC CDR2 comprising SEQ ID No: 239 or a sequence with at least 80% sequence identity thereto and a LC CDR3 comprising SEQ ID No: 240 or a sequence with at least 80% sequence identity thereto;
an antibody comprising a HC CDR1 comprising SEQ ID No: 245 or a sequence with at least 80% sequence identity thereto, a HC CDR2 comprising SEQ ID No: 246 or a sequence with at least 80% sequence identity thereto, a HC CDR3 comprising SEQ ID No: 247 or a sequence with at least 80% sequence identity thereto, a LC CDR1 comprising SEQ ID No: 248 or a sequence with at least 80% sequence identity thereto, a LC CDR2 comprising SEQ ID No: 249 or a sequence with at least 80% sequence identity thereto and a LC CDR3 comprising SEQ ID No: 250 or a sequence with at least 80% sequence identity thereto;
an antibody comprising a HC CDR1 comprising SEQ ID No: 255 or a sequence with at least 80% sequence identity thereto, a HC CDR2 comprising SEQ ID No: 256 or a sequence with at least 80% sequence identity thereto, a HC CDR3 comprising SEQ ID No: 257 or a sequence with at least 80% sequence identity thereto, a LC CDR1 comprising SEQ ID No: 258 or a sequence with at least 80% sequence identity thereto, a LC CDR2 comprising SEQ ID No: 259 or a sequence with at least 80% sequence identity thereto and a LC CDR3 comprising SEQ ID No: 260 or a sequence with at least 80% sequence identity thereto;
an antibody comprising a HC CDR1 comprising SEQ ID No: 265 or a sequence with at least 80% sequence identity thereto, a HC CDR2 comprising SEQ ID No: 266 or a sequence with at least 80% sequence identity thereto, a HC CDR3 comprising SEQ ID No: 267 or a sequence with at least 80% sequence identity thereto, a LC CDR1 comprising SEQ ID No: 268 or a sequence with at least 80% sequence identity thereto, a LC CDR2 comprising SEQ ID No: 269 or a sequence with at least 80% sequence identity thereto and a LC CDR3 comprising SEQ ID No: 270 or a sequence with at least 80% sequence identity thereto;
an antibody comprising a HC CDR1 comprising SEQ ID No: 285 or a sequence with at least 80% sequence identity thereto, a HC CDR2 comprising SEQ ID No: 286 or a sequence with at least 80% sequence identity thereto, a HC CDR3 comprising SEQ ID No: 287 or a sequence with at least 80% sequence identity thereto, a LC CDR1 comprising SEQ ID No: 288 or a sequence with at least 80% sequence identity thereto, a LC CDR2 comprising SEQ ID No: 289 or a sequence with at least 80% sequence identity thereto and a LC CDR3 comprising SEQ ID No: 290 or a sequence with at least 80% sequence identity thereto;
an antibody comprising a HC CDR1 comprising SEQ ID No: 295 or a sequence with at least 80% sequence identity thereto, a HC CDR2 comprising SEQ ID No: 296 or a sequence with at least 80% sequence identity thereto, a HC CDR3 comprising SEQ ID No: 297 or a sequence with at least 80% sequence identity thereto, a LC CDR1 comprising SEQ ID No: 298 or a sequence with at least 80% sequence identity thereto, a LC CDR2 comprising SEQ ID No: 299 or a sequence with at least 80% sequence identity thereto and a LC CDR3 comprising SEQ ID No: 300 or a sequence with at least 80% sequence identity thereto;
an antibody comprising a HC CDR1 comprising SEQ ID No: 305 or a sequence with at least 80% sequence identity thereto, a HC CDR2 comprising SEQ ID No: 306 or a sequence with at least 80% sequence identity thereto, a HC CDR3 comprising SEQ ID No: 307 or a sequence with at least 80% sequence identity thereto, a LC CDR1 comprising SEQ ID No: 308 or a sequence with at least 80% sequence identity thereto, a LC CDR2 comprising SEQ ID No: 309 or a sequence with at least 80% sequence identity thereto and a LC CDR3 comprising SEQ ID No: 310 or a sequence with at least 80% sequence identity thereto;
an antibody comprising a HC CDR1 comprising SEQ ID No: 315 or a sequence with at least 80% sequence identity thereto, a HC CDR2 comprising SEQ ID No: 316 or a sequence with at least 80% sequence identity thereto, a HC CDR3 comprising SEQ ID No: 317 or a sequence with at least 80% sequence identity thereto, a LC CDR1 comprising SEQ ID No: 318 or a sequence with at least 80% sequence identity thereto, a LC CDR2 comprising SEQ ID No: 319 or a sequence with at least 80% sequence identity thereto and a LC CDR3 comprising SEQ ID No: 320 or a sequence with at least 80% sequence identity thereto;
an antibody comprising a HC CDR1 comprising SEQ ID No: 325 or a sequence with at least 80% sequence identity thereto, a HC CDR2 comprising SEQ ID No: 326 or a sequence with at least 80% sequence identity thereto, a HC CDR3 comprising SEQ ID No: 327 or a sequence with at least 80% sequence identity thereto, a LC CDR1 comprising SEQ ID No: 328 or a sequence with at least 80% sequence identity thereto, a LC CDR2 comprising SEQ ID No: 329 or a sequence with at least 80% sequence identity thereto and a LC CDR3 comprising SEQ ID No: 330 or a sequence with at least 80% sequence identity thereto;
an antibody comprising a HC CDR1 comprising SEQ ID No: 335 or a sequence with at least 80% sequence identity thereto, a HC CDR2 comprising SEQ ID No: 336 or a sequence with at least 80% sequence identity thereto, a HC CDR3 comprising SEQ ID No: 337 or a sequence with at least 80% sequence identity thereto, a LC CDR1 comprising SEQ ID No: 338 or a sequence with at least 80% sequence identity thereto, a LC CDR2 comprising SEQ ID No: 339 or a sequence with at least 80% sequence identity thereto and a LC CDR3 comprising SEQ ID No: 340 or a sequence with at least 80% sequence identity thereto;
an antibody comprising a HC CDR1 comprising SEQ ID No: 345 or a sequence with at least 80% sequence identity thereto, a HC CDR2 comprising SEQ ID No: 346 or a sequence with at least 80% sequence identity thereto, a HC CDR3 comprising SEQ ID No: 347 or a sequence with at least 80% sequence identity thereto, a LC CDR1 comprising SEQ ID No: 348 or a sequence with at least 80% sequence identity thereto, a LC CDR2 comprising SEQ ID No: 349 or a sequence with at least 80% sequence identity thereto and a LC CDR3 comprising SEQ ID No: 350 or a sequence with at least 80% sequence identity thereto;
an antibody comprising a HC CDR1 comprising SEQ ID No: 355 or a sequence with at least 80% sequence identity thereto, a HC CDR2 comprising SEQ ID No: 356 or a sequence with at least 80% sequence identity thereto, a HC CDR3 comprising SEQ ID No: 357 or a sequence with at least 80% sequence identity thereto, a LC CDR1 comprising SEQ ID No: 358 or a sequence with at least 80% sequence identity thereto, a LC CDR2 comprising SEQ ID No: 359 or a sequence with at least 80% sequence identity thereto and a LC CDR3 comprising SEQ ID No: 360 or a sequence with at least 80% sequence identity thereto;
an antibody comprising a HC CDR1 comprising SEQ ID No: 365 or a sequence with at least 80% sequence identity thereto, a HC CDR2 comprising SEQ ID No: 366 or a sequence with at least 80% sequence identity thereto, a HC CDR3 comprising SEQ ID No: 367 or a sequence with at least 80% sequence identity thereto, a LC CDR1 comprising SEQ ID No: 368 or a sequence with at least 80% sequence identity thereto, a LC CDR2 comprising SEQ ID No: 369 or a sequence with at least 80% sequence identity thereto and a LC CDR3 comprising SEQ ID No: 370 or a sequence with at least 80% sequence identity thereto;
an antibody comprising a HC CDR1 comprising SEQ ID No: 375 or a sequence with at least 80% sequence identity thereto, a HC CDR2 comprising SEQ ID No: 376 or a sequence with at least 80% sequence identity thereto, a HC CDR3 comprising SEQ ID No: 377 or a sequence with at least 80% sequence identity thereto, a LC CDR1 comprising SEQ ID No: 378 or a sequence with at least 80% sequence identity thereto, a LC CDR2 comprising SEQ ID No: 379 or a sequence with at least 80% sequence identity thereto and a LC CDR3 comprising SEQ ID No: 380 or a sequence with at least 80% sequence identity thereto;
an antibody comprising a HC CDR1 comprising SEQ ID No: 385 or a sequence with at least 80% sequence identity thereto, a HC CDR2 comprising SEQ ID No: 386 or a sequence with at least 80% sequence identity thereto, a HC CDR3 comprising SEQ ID No: 387 or a sequence with at least 80% sequence identity thereto, a LC CDR1 comprising SEQ ID No: 388 or a sequence with at least 80% sequence identity thereto, a LC CDR2 comprising SEQ ID No: 389 or a sequence with at least 80% sequence identity thereto and a LC CDR3 comprising SEQ ID No: 390 or a sequence with at least 80% sequence identity thereto;
an antibody comprising a HC CDR1 comprising SEQ ID No: 405 or a sequence with at least 80% sequence identity thereto, a HC CDR2 comprising SEQ ID No: 406 or a sequence with at least 80% sequence identity thereto, a HC CDR3 comprising SEQ ID No: 407 or a sequence with at least 80% sequence identity thereto, a LC CDR1 comprising SEQ ID No: 408 or a sequence with at least 80% sequence identity thereto, a LC CDR2 comprising SEQ ID No: 409 or a sequence with at least 80% sequence identity thereto and a LC CDR3 comprising SEQ ID No: 410 or a sequence with at least 80% sequence identity thereto;
an antibody comprising a HC CDR1 comprising SEQ ID No: 415 or a sequence with at least 80% sequence identity thereto, a HC CDR2 comprising SEQ ID No: 416 or a sequence with at least 80% sequence identity thereto, a HC CDR3 comprising SEQ ID No: 417 or a sequence with at least 80% sequence identity thereto, a LC CDR1 comprising SEQ ID No: 418 or a sequence with at least 80% sequence identity thereto, a LC CDR2 comprising SEQ ID No: 419 or a sequence with at least 80% sequence identity thereto and a LC CDR3 comprising SEQ ID No: 420 or a sequence with at least 80% sequence identity thereto;
an antibody comprising a HC CDR1 comprising SEQ ID No: 425 or a sequence with at least 80% sequence identity thereto, a HC CDR2 comprising SEQ ID No: 426 or a sequence with at least 80% sequence identity thereto, a HC CDR3 comprising SEQ ID No: 427 or a sequence with at least 80% sequence identity thereto, a LC CDR1 comprising SEQ ID No: 428 or a sequence with at least 80% sequence identity thereto, a LC CDR2 comprising SEQ ID No: 429 or a sequence with at least 80% sequence identity thereto and a LC CDR3 comprising SEQ ID No: 430 or a sequence with at least 80% sequence identity thereto;
an antibody comprising a HC CDR1 comprising SEQ ID No: 435 or a sequence with at least 80% sequence identity thereto, a HC CDR2 comprising SEQ ID No: 436 or a sequence with at least 80% sequence identity thereto, a HC CDR3 comprising SEQ ID No: 437 or a sequence with at least 80% sequence identity thereto, a LC CDR1 comprising SEQ ID No: 438 or a sequence with at least 80% sequence identity thereto, a LC CDR2 comprising SEQ ID No: 439 or a sequence with at least 80% sequence identity thereto and a LC CDR3 comprising SEQ ID No: 440 or a sequence with at least 80% sequence identity thereto;
an antibody comprising a HC CDR1 comprising SEQ ID No: 445 or a sequence with at least 80% sequence identity thereto, a HC CDR2 comprising SEQ ID No: 446 or a sequence with at least 80% sequence identity thereto, a HC CDR3 comprising SEQ ID No: 447 or a sequence with at least 80% sequence identity thereto, a LC CDR1 comprising SEQ ID No: 448 or a sequence with at least 80% sequence identity thereto, a LC CDR2 comprising SEQ ID No: 449 or a sequence with at least 80% sequence identity thereto and a LC CDR3 comprising SEQ ID No: 440 or a sequence with at least 80% sequence identity thereto;
an antibody comprising a HC CDR1 comprising SEQ ID No: 455 or a sequence with at least 80% sequence identity thereto, a HC CDR2 comprising SEQ ID No: 456 or a sequence with at least 80% sequence identity thereto, a HC CDR3 comprising SEQ ID No: 457 or a sequence with at least 80% sequence identity thereto, a LC CDR1 comprising SEQ ID No: 458 or a sequence with at least 80% sequence identity thereto, a LC CDR2 comprising SEQ ID No: 459 or a sequence with at least 80% sequence identity thereto and a LC CDR3 comprising SEQ ID No: 460 or a sequence with at least 80% sequence identity thereto;
an antibody comprising a HC CDR1 comprising SEQ ID No: 465 or a sequence with at least 80% sequence identity thereto, a HC CDR2 comprising SEQ ID No: 466 or a sequence with at least 80% sequence identity thereto, a HC CDR3 comprising SEQ ID No: 467 or a sequence with at least 80% sequence identity thereto, a LC CDR1 comprising SEQ ID No: 468 or a sequence with at least 80% sequence identity thereto, a LC CDR2 comprising SEQ ID No: 469 or a sequence with at least 80% sequence identity thereto and a LC CDR3 comprising SEQ ID No: 470 or a sequence with at least 80% sequence identity thereto;
an antibody comprising a HC CDR1 comprising SEQ ID No: 475 or a sequence with at least 80% sequence identity thereto, a HC CDR2 comprising SEQ ID No: 476 or a sequence with at least 80% sequence identity thereto, a HC CDR3 comprising SEQ ID No: 477 or a sequence with at least 80% sequence identity thereto, a LC CDR1 comprising SEQ ID No: 478 or a sequence with at least 80% sequence identity thereto, a LC CDR2 comprising SEQ ID No: 479 or a sequence with at least 80% sequence identity thereto and a LC CDR3 comprising SEQ ID No: 480 or a sequence with at least 80% sequence identity thereto;
an antibody comprising a HC CDR1 comprising SEQ ID No: 485 or a sequence with at least 80% sequence identity thereto, a HC CDR2 comprising SEQ ID No: 486 or a sequence with at least 80% sequence identity thereto, a HC CDR3 comprising SEQ ID No: 487 or a sequence with at least 80% sequence identity thereto, a LC CDR1 comprising SEQ ID No: 488 or a sequence with at least 80% sequence identity thereto, a LC CDR2 comprising SEQ ID No: 489 or a sequence with at least 80% sequence identity thereto and a LC CDR3 comprising SEQ ID No: 490 or a sequence with at least 80% sequence identity thereto;
an antibody comprising a HC CDR1 comprising SEQ ID No: 495 or a sequence with at least 80% sequence identity thereto, a HC CDR2 comprising SEQ ID No: 496 or a sequence with at least 80% sequence identity thereto, a HC CDR3 comprising SEQ ID No: 497 or a sequence with at least 80% sequence identity thereto, a LC CDR1 comprising SEQ ID No: 498 or a sequence with at least 80% sequence identity thereto, a LC CDR2 comprising SEQ ID No: 499 or a sequence with at least 80% sequence identity thereto and a LC CDR3 comprising SEQ ID No: 500 or a sequence with at least 80% sequence identity thereto;
an antibody comprising a HC CDR1 comprising SEQ ID No: 505 or a sequence with at least 80% sequence identity thereto, a HC CDR2 comprising SEQ ID No: 506 or a sequence with at least 80% sequence identity thereto, a HC CDR3 comprising SEQ ID No: 507 or a sequence with at least 80% sequence identity thereto, a LC CDR1 comprising SEQ ID No: 508 or a sequence with at least 80% sequence identity thereto, a LC CDR2 comprising SEQ ID No: 509 or a sequence with at least 80% sequence identity thereto and a LC CDR3 comprising SEQ ID No: 510 or a sequence with at least 80% sequence identity thereto;
an antibody comprising a HC CDR1 comprising SEQ ID No: 515 or a sequence with at least 80% sequence identity thereto, a HC CDR2 comprising SEQ ID No: 516 or a sequence with at least 80% sequence identity thereto, a HC CDR3 comprising SEQ ID No: 517 or a sequence with at least 80% sequence identity thereto, a LC CDR1 comprising SEQ ID No: 518 or a sequence with at least 80% sequence identity thereto, a LC CDR2 comprising SEQ ID No: 519 or a sequence with at least 80% sequence identity thereto and a LC CDR3 comprising SEQ ID No: 520 or a sequence with at least 80% sequence identity thereto;
an antibody comprising a HC CDR1 comprising SEQ ID No: 525 or a sequence with at least 80% sequence identity thereto, a HC CDR2 comprising SEQ ID No: 526 or a sequence with at least 80% sequence identity thereto, a HC CDR3 comprising SEQ ID No: 527 or a sequence with at least 80% sequence identity thereto, a LC CDR1 comprising SEQ ID No: 528 or a sequence with at least 80% sequence identity thereto, a LC CDR2 comprising SEQ ID No: 529 or a sequence with at least 80% sequence identity thereto and a LC CDR3 comprising SEQ ID No: 530 or a sequence with at least 80% sequence identity thereto;
an antibody comprising a HC CDR1 comprising SEQ ID No: 535 or a sequence with at least 80% sequence identity thereto, a HC CDR2 comprising SEQ ID No: 536 or a sequence with at least 80% sequence identity thereto, a HC CDR3 comprising SEQ ID No: 537 or a sequence with at least 80% sequence identity thereto, a LC CDR1 comprising SEQ ID No: 538 or a sequence with at least 80% sequence identity thereto, a LC CDR2 comprising SEQ ID No: 539 or a sequence with at least 80% sequence identity thereto and a LC CDR3 comprising SEQ ID No: 540 or a sequence with at least 80% sequence identity thereto;
an antibody comprising a HC CDR1 comprising SEQ ID No: 545 or a sequence with at least 80% sequence identity thereto, a HC CDR2 comprising SEQ ID No: 546 or a sequence with at least 80% sequence identity thereto, a HC CDR3 comprising SEQ ID No: 547 or a sequence with at least 80% sequence identity thereto, a LC CDR1 comprising SEQ ID No: 548 or a sequence with at least 80% sequence identity thereto, a LC CDR2 comprising SEQ ID No: 549 or a sequence with at least 80% sequence identity thereto and a LC CDR3 comprising SEQ ID No: 550 or a sequence with at least 80% sequence identity thereto;
an antibody comprising a HC CDR1 comprising SEQ ID No: 555 or a sequence with at least 80% sequence identity thereto, a HC CDR2 comprising SEQ ID No: 556 or a sequence with at least 80% sequence identity thereto, a HC CDR3 comprising SEQ ID No: 557 or a sequence with at least 80% sequence identity thereto, a LC CDR1 comprising SEQ ID No: 558 or a sequence with at least 80% sequence identity thereto, a LC CDR2 comprising SEQ ID No: 559 or a sequence with at least 80% sequence identity thereto and a LC CDR3 comprising SEQ ID No: 560 or a sequence with at least 80% sequence identity thereto;
an antibody comprising a HC CDR1 comprising SEQ ID No: 565 or a sequence with at least 80% sequence identity thereto, a HC CDR2 comprising SEQ ID No: 566 or a sequence with at least 80% sequence identity thereto, a HC CDR3 comprising SEQ ID No: 567 or a sequence with at least 80% sequence identity thereto, a LC CDR1 comprising SEQ ID No: 568 or a sequence with at least 80% sequence identity thereto, a LC CDR2 comprising SEQ ID No: 569 or a sequence with at least 80% sequence identity thereto and a LC CDR3 comprising SEQ ID No: 570 or a sequence with at least 80% sequence identity thereto;
an antibody comprising a HC CDR1 comprising SEQ ID No: 585 or a sequence with at least 80% sequence identity thereto, a HC CDR2 comprising SEQ ID No: 586 or a sequence with at least 80% sequence identity thereto, a HC CDR3 comprising SEQ ID No: 587 or a sequence with at least 80% sequence identity thereto, a LC CDR1 comprising SEQ ID No: 588 or a sequence with at least 80% sequence identity thereto, a LC CDR2 comprising SEQ ID No: 589 or a sequence with at least 80% sequence identity thereto and a LC CDR3 comprising SEQ ID No: 590 or a sequence with at least 80% sequence identity thereto;
an antibody comprising a HC CDR1 comprising SEQ ID No: 595 or a sequence with at least 80% sequence identity thereto, a HC CDR2 comprising SEQ ID No: 596 or a sequence with at least 80% sequence identity thereto, a HC CDR3 comprising SEQ ID No: 597 or a sequence with at least 80% sequence identity thereto, a LC CDR1 comprising SEQ ID No: 598 or a sequence with at least 80% sequence identity thereto, a LC CDR2 comprising SEQ ID No: 599 or a sequence with at least 80% sequence identity thereto and a LC CDR3 comprising SEQ ID No: 600 or a sequence with at least 80% sequence identity thereto;
an antibody comprising a HC CDR1 comprising SEQ ID No: 605 or a sequence with at least 80% sequence identity thereto, a HC CDR2 comprising SEQ ID No: 606 or a sequence with at least 80% sequence identity thereto, a HC CDR3 comprising SEQ ID No: 607 or a sequence with at least 80% sequence identity thereto, a LC CDR1 comprising SEQ ID No: 608 or a sequence with at least 80% sequence identity thereto, a LC CDR2 comprising SEQ ID No: 609 or a sequence with at least 80% sequence identity thereto and a LC CDR3 comprising SEQ ID No: 610 or a sequence with at least 80% sequence identity thereto;
an antibody comprising a HC CDR1 comprising SEQ ID No: 615 or a sequence with at least 80% sequence identity thereto, a HC CDR2 comprising SEQ ID No: 616 or a sequence with at least 80% sequence identity thereto, a HC CDR3 comprising SEQ ID No: 617 or a sequence with at least 80% sequence identity thereto, a LC CDR1 comprising SEQ ID No: 618 or a sequence with at least 80% sequence identity thereto, a LC CDR2 comprising SEQ ID No: 619 or a sequence with at least 80% sequence identity thereto and a LC CDR3 comprising SEQ ID No: 620 or a sequence with at least 80% sequence identity thereto;
an antibody comprising a HC CDR1 comprising SEQ ID No: 625 or a sequence with at least 80% sequence identity thereto, a HC CDR2 comprising SEQ ID No: 626 or a sequence with at least 80% sequence identity thereto, a HC CDR3 comprising SEQ ID No: 627 or a sequence with at least 80% sequence identity thereto, a LC CDR1 comprising SEQ ID No: 628 or a sequence with at least 80% sequence identity thereto, a LC CDR2 comprising SEQ ID No: 629 or a sequence with at least 80% sequence identity thereto and a LC CDR3 comprising SEQ ID No: 630 or a sequence with at least 80% sequence identity thereto;
an antibody comprising a HC CDR1 comprising SEQ ID No: 635 or a sequence with at least 80% sequence identity thereto, a HC CDR2 comprising SEQ ID No: 636 or a sequence with at least 80% sequence identity thereto, a HC CDR3 comprising SEQ ID No: 637 or a sequence with at least 80% sequence identity thereto, a LC CDR1 comprising SEQ ID No: 638 or a sequence with at least 80% sequence identity thereto, a LC CDR2 comprising SEQ ID No: 639 or a sequence with at least 80% sequence identity thereto and a LC CDR3 comprising SEQ ID No: 640 or a sequence with at least 80% sequence identity thereto;
an antibody comprising a HC CDR1 comprising SEQ ID No: 645 or a sequence with at least 80% sequence identity thereto, a HC CDR2 comprising SEQ ID No: 646 or a sequence with at least 80% sequence identity thereto, a HC CDR3 comprising SEQ ID No: 647 or a sequence with at least 80% sequence identity thereto, a LC CDR1 comprising SEQ ID No: 648 or a sequence with at least 80% sequence identity thereto, a LC CDR2 comprising SEQ ID No: 649 or a sequence with at least 80% sequence identity thereto and a LC CDR3 comprising SEQ ID No: 650 or a sequence with at least 80% sequence identity thereto;
an antibody comprising a HC CDR1 comprising SEQ ID No: 655 or a sequence with at least 80% sequence identity thereto, a HC CDR2 comprising SEQ ID No: 656 or a sequence with at least 80% sequence identity thereto, a HC CDR3 comprising SEQ ID No: 657 or a sequence with at least 80% sequence identity thereto, a LC CDR1 comprising SEQ ID No: 658 or a sequence with at least 80% sequence identity thereto, a LC CDR2 comprising SEQ ID No: 659 or a sequence with at least 80% sequence identity thereto and a LC CDR3 comprising SEQ ID No: 660 or a sequence with at least 80% sequence identity thereto;
an antibody comprising a HC CDR1 comprising SEQ ID No: 665 or a sequence with at least 80% sequence identity thereto, a HC CDR2 comprising SEQ ID No: 666 or a sequence with at least 80% sequence identity thereto, a HC CDR3 comprising SEQ ID No: 667 or a sequence with at least 80% sequence identity thereto, a LC CDR1 comprising SEQ ID No: 668 or a sequence with at least 80% sequence identity thereto, a LC CDR2 comprising SEQ ID No: 669 or a sequence with at least 80% sequence identity thereto and a LC CDR3 comprising SEQ ID No: 670 or a sequence with at least 80% sequence identity thereto;
an antibody comprising a HC CDR1 comprising SEQ ID No: 675 or a sequence with at least 80% sequence identity thereto, a HC CDR2 comprising SEQ ID No: 676 or a sequence with at least 80% sequence identity thereto, a HC CDR3 comprising SEQ ID No: 677 or a sequence with at least 80% sequence identity thereto, a LC CDR1 comprising SEQ ID No: 678 or a sequence with at least 80% sequence identity thereto, a LC CDR2 comprising SEQ ID No: 679 or a sequence with at least 80% sequence identity thereto and a LC CDR3 comprising SEQ ID No: 680 or a sequence with at least 80% sequence identity thereto;
an antibody comprising a HC CDR1 comprising SEQ ID No: 685 or a sequence with at least 80% sequence identity thereto, a HC CDR2 comprising SEQ ID No: 686 or a sequence with at least 80% sequence identity thereto, a HC CDR3 comprising SEQ ID No: 687 or a sequence with at least 80% sequence identity thereto, a LC CDR1 comprising SEQ ID No: 688 or a sequence with at least 80% sequence identity thereto, a LC CDR2 comprising SEQ ID No: 689 or a sequence with at least 80% sequence identity thereto and a LC CDR3 comprising SEQ ID No: 690 or a sequence with at least 80% sequence identity thereto;
an antibody comprising a HC CDR1 comprising SEQ ID No: 695 or a sequence with at least 80% sequence identity thereto, a HC CDR2 comprising SEQ ID No: 696 or a sequence with at least 80% sequence identity thereto, a HC CDR3 comprising SEQ ID No: 697 or a sequence with at least 80% sequence identity thereto, a LC CDR1 comprising SEQ ID No: 698 or a sequence with at least 80% sequence identity thereto, a LC CDR2 comprising SEQ ID No: 699 or a sequence with at least 80% sequence identity thereto and a LC CDR3 comprising SEQ ID No: 700 or a sequence with at least 80% sequence identity thereto;
an antibody comprising a HC CDR1 comprising SEQ ID No: 705 or a sequence with at least 80% sequence identity thereto, a HC CDR2 comprising SEQ ID No: 706 or a sequence with at least 80% sequence identity thereto, a HC CDR3 comprising SEQ ID No: 707 or a sequence with at least 80% sequence identity thereto, a LC CDR1 comprising SEQ ID No: 708 or a sequence with at least 80% sequence identity thereto, a LC CDR2 comprising SEQ ID No: 709 or a sequence with at least 80% sequence identity thereto and a LC CDR3 comprising SEQ ID No: 710 or a sequence with at least 80% sequence identity thereto;
an antibody comprising a HC CDR1 comprising SEQ ID No: 715 or a sequence with at least 80% sequence identity thereto, a HC CDR2 comprising SEQ ID No: 716 or a sequence with at least 80% sequence identity thereto, a HC CDR3 comprising SEQ ID No: 717 or a sequence with at least 80% sequence identity thereto, a LC CDR1 comprising SEQ ID No: 718 or a sequence with at least 80% sequence identity thereto, a LC CDR2 comprising SEQ ID No: 719 or a sequence with at least 80% sequence identity thereto and a LC CDR3 comprising SEQ ID No: 720 or a sequence with at least 80% sequence identity thereto;
an antibody comprising a HC CDR1 comprising SEQ ID No: 751 or a sequence with at least 80% sequence identity thereto, a HC CDR2 comprising SEQ ID No: 752 or a sequence with at least 80% sequence identity thereto, a HC CDR3 comprising SEQ ID No: 753 or a sequence with at least 80% sequence identity thereto, a LC CDR1 comprising SEQ ID No: 754 or a sequence with at least 80% sequence identity thereto, a LC CDR2 comprising SEQ ID No: 755 or a sequence with at least 80% sequence identity thereto and a LC CDR3 comprising SEQ ID No: 756 or a sequence with at least 80% sequence identity thereto;
an antibody comprising a HC CDR1 comprising SEQ ID No: 761 or a sequence with at least 80% sequence identity thereto, a HC CDR2 comprising SEQ ID No: 762 or a sequence with at least 80% sequence identity thereto, a HC CDR3 comprising SEQ ID No: 763 or a sequence with at least 80% sequence identity thereto, a LC CDR1 comprising SEQ ID No: 764 or a sequence with at least 80% sequence identity thereto, a LC CDR2 comprising SEQ ID No: 765 or a sequence with at least 80% sequence identity thereto and a LC CDR3 comprising SEQ ID No: 766 or a sequence with at least 80% sequence identity thereto;
an antibody comprising a HC CDR1 comprising SEQ ID No: 771 or a sequence with at least 80% sequence identity thereto, a HC CDR2 comprising SEQ ID No: 772 or a sequence with at least 80% sequence identity thereto, a HC CDR3 comprising SEQ ID No: 773 or a sequence with at least 80% sequence identity thereto, a LC CDR1 comprising SEQ ID No: 774 or a sequence with at least 80% sequence identity thereto, a LC CDR2 comprising SEQ ID No: 775 or a sequence with at least 80% sequence identity thereto and a LC CDR3 comprising SEQ ID No: 776 or a sequence with at least 80% sequence identity thereto;
an antibody comprising a HC CDR1 comprising SEQ ID No: 781 or a sequence with at least 80% sequence identity thereto, a HC CDR2 comprising SEQ ID No: 782 or a sequence with at least 80% sequence identity thereto, a HC CDR3 comprising SEQ ID No: 783 or a sequence with at least 80% sequence identity thereto, a LC CDR1 comprising SEQ ID No: 784 or a sequence with at least 80% sequence identity thereto, a LC CDR2 comprising SEQ ID No: 785 or a sequence with at least 80% sequence identity thereto and a LC CDR3 comprising SEQ ID No: 786 or a sequence with at least 80% sequence identity thereto;
an antibody comprising a HC CDR1 comprising SEQ ID No: 791 or a sequence with at least 80% sequence identity thereto, a HC CDR2 comprising SEQ ID No: 792 or a sequence with at least 80% sequence identity thereto, a HC CDR3 comprising SEQ ID No: 793 or a sequence with at least 80% sequence identity thereto, a LC CDR1 comprising SEQ ID No: 794 or a sequence with at least 80% sequence identity thereto, a LC CDR2 comprising SEQ ID No: 795 or a sequence with at least 80% sequence identity thereto and a LC CDR3 comprising SEQ ID No: 796 or a sequence with at least 80% sequence identity thereto; and
an antibody comprising a HC CDR1 comprising SEQ ID No: 801 or a sequence with at least 80% sequence identity thereto, a HC CDR2 comprising SEQ ID No: 802 or a sequence with at least 80% sequence identity thereto, a HC CDR3 comprising SEQ ID No: 803 or a sequence with at least 80% sequence identity thereto, a LC CDR1 comprising SEQ ID No: 804 or a sequence with at least 80% sequence identity thereto, a LC CDR2 comprising SEQ ID No: 805 or a sequence with at least 80% sequence identity thereto and a LC CDR3 comprising SEQ ID No: 806 or a sequence with at least 80% sequence identity thereto.

3. The antibody or fragment according to claim 2 wherein said companion animal is a dog or a cat.

4. The antibody or fragment according to claim 2 wherein the antibody or fragment binds to canine OX40L.

5. The antibody or fragment according to claim 4 wherein said antibody or fragment is capable of:
a) reducing, inhibiting or neutralising OX40 activity or activation in the companion animal or in a cell of the companion animal;
b) modifying secretion of a cytokine in the companion animal or in a cell of the companion animal and/or c) decreasing proliferation of leukocytes in the companion animal.

6. The antibody or fragment according to claim 5 wherein the antibody is a canine or caninized antibody.

7. An antibody or antibody fragment that specifically binds to companion animal OX40L wherein the antibody is selected from the group consisting of:
an antibody comprising a HC variable region comprising or consisting of SEQ ID No. 12 or a sequence with at least 80% sequence identity thereto and a LC variable region comprising or consisting of SEQ ID No. 14 or a sequence with at least 80% sequence identity thereto;
an antibody comprising a HC variable region comprising or consisting of SEQ ID No. 22 or a sequence with at least 80% sequence identity thereto and a LC variable region comprising or consisting of SEQ ID No. 24 or a sequence with at least 80% sequence identity thereto;
an antibody comprising a HC variable region comprising or consisting of SEQ ID No. 32 or a sequence with at least 80% sequence identity thereto and a LC variable region comprising or consisting of SEQ ID No. 34 or a sequence with at least 80% sequence identity thereto;
an antibody comprising a HC variable region comprising or consisting of SEQ ID No. 42 or a sequence with at least 80% sequence identity thereto and a LC variable region comprising or consisting of SEQ ID No. 44 or a sequence with at least 80% sequence identity thereto;
an antibody comprising a HC variable region comprising or consisting of SEQ ID No. 52 or a sequence with at least 80% sequence identity thereto and a LC variable region comprising or consisting of SEQ ID No. 54 or a sequence with at least 80% sequence identity thereto;
an antibody comprising a HC variable region comprising or consisting of SEQ ID No. 62 or a sequence with at least 80% sequence identity thereto and a LC variable region comprising or consisting of SEQ ID No. 64 or a sequence with at least 80% sequence identity thereto;
an antibody comprising a HC variable region comprising or consisting of SEQ ID No. 72 or a sequence with at least 80% sequence identity thereto and a LC variable region comprising or consisting of SEQ ID No. 7 4 or a sequence with at least 80% sequence identity thereto;
an antibody comprising a HC variable region comprising or consisting of SEQ ID No. 82 or a sequence with at least 80% sequence identity thereto and a LC variable region comprising or consisting of SEQ ID No. 84 or a sequence with at least 80% sequence identity thereto;
an antibody comprising a HC variable region comprising or consisting of SEQ ID No. 92 or a sequence with at least 80% sequence identity thereto and a LC variable region comprising or consisting of SEQ ID No. 94 or a sequence with at least 80% sequence identity thereto;
an antibody comprising a HC variable region comprising or consisting of SEQ ID No. 102 or a sequence with at least 80% sequence identity thereto and a LC variable region comprising or consisting of SEQ ID No. 104 or a sequence with at least 80% sequence identity thereto;
an antibody comprising a HC variable region comprising or consisting of SEQ ID No. 112 or a sequence with at least 80% sequence identity thereto and a LC variable region comprising or consisting of SEQ ID No. 114 or a sequence with at least 80% sequence identity thereto;
an antibody comprising a HC variable region comprising or consisting of SEQ ID No. 122 or a sequence with at least 80% sequence identity thereto and a LC variable region comprising or consisting of SEQ ID No. 124 or a sequence with at least 80% sequence identity thereto;
an antibody comprising a HC variable region comprising or consisting of SEQ ID No. 132 or a sequence with at least 80% sequence identity thereto and a LC variable region comprising or consisting of SEQ ID No. 134 or a sequence with at least 80% sequence identity thereto;
an antibody comprising a HC variable region comprising or consisting of SEQ ID No. 142 or a sequence with at least 80% sequence identity thereto and a LC variable region comprising or consisting of SEQ ID No. 144 or a sequence with at least 80% sequence identity thereto;
an antibody comprising a HC variable region comprising or consisting of SEQ ID No. 152 or a sequence with at least 80% sequence identity thereto and a LC variable region comprising or consisting of SEQ ID No. 154 or a sequence with at least 80% sequence identity thereto;
an antibody comprising a HC variable region comprising or consisting of SEQ ID No. 162 or a sequence with at least 80% sequence identity thereto and a LC variable region comprising or consisting of SEQ ID No. 164 or a sequence with at least 80% sequence identity thereto;
an antibody comprising a HC variable region comprising or consisting of SEQ ID No. 172 or a sequence with at least 80% sequence identity thereto and a LC variable region comprising or consisting of SEQ ID No. 174 or a sequence with at least 80% sequence identity thereto;
an antibody comprising a HC variable region comprising or consisting of SEQ ID No. 182 or a sequence with at least 80% sequence identity thereto and a LC variable region comprising or consisting of SEQ ID No. 184 or a sequence with at least 80% sequence identity thereto;
an antibody comprising a HC variable region comprising or consisting of SEQ ID No.192 or a sequence with at least 80% sequence identity thereto and a LC variable region comprising or consisting of SEQ ID No. 194 or a sequence with at least 80% sequence identity thereto;
an antibody comprising a HC variable region comprising or consisting of SEQ ID No. 202 or a sequence with at least 80% sequence identity thereto and a LC variable region comprising or consisting of SEQ ID No. 204 or a sequence with at least 80% sequence identity thereto;
an antibody comprising a HC variable region comprising or consisting of SEQ ID No. 212 or a sequence with at least 80% sequence identity thereto and a LC variable region comprising or consisting of SEQ ID No. 214 or a sequence with at least 80% sequence identity thereto;
an antibody comprising a HC variable region comprising or consisting of SEQ ID No. 222 or a sequence with at least 80% sequence identity thereto and a LC variable region comprising or consisting of SEQ ID No. 224 or a sequence with at least 80% sequence identity thereto;
an antibody comprising a HC variable region comprising or consisting of SEQ ID No. 232 or a sequence with at least 80% sequence identity thereto and a LC variable region comprising or consisting of SEQ ID No. 234 or a sequence with at least 80% sequence identity thereto;
an antibody comprising a HC variable region comprising or consisting of SEQ ID No. 242 or a sequence with at least 80% sequence identity thereto and a LC variable region comprising or consisting of SEQ ID No. 244 or a sequence with at least 80% sequence identity thereto;
an antibody comprising a HC variable region comprising or consisting of SEQ ID No. 252 or a sequence with at least 80% sequence identity thereto and a LC variable region comprising or consisting of SEQ ID No. 254 or a sequence with at least 80% sequence identity thereto;
an antibody comprising a HC variable region comprising or consisting of SEQ ID No. 262 or a sequence with at least 80% sequence identity thereto and a LC variable region comprising or consisting of SEQ ID No. 264 or a sequence with at least 80% sequence identity thereto;
an antibody comprising a HC variable region comprising or consisting of SEQ ID No. 272 or a sequence with at least 80% sequence identity thereto and a LC variable region comprising or consisting of SEQ ID No. 274 or a sequence with at least 80% sequence identity thereto;
an antibody comprising a HC variable region comprising or consisting of SEQ ID No. 282 or a sequence with at least 80% sequence identity thereto and a LC variable region comprising or consisting of SEQ ID No. 284 or a sequence with at least 80% sequence identity thereto;
an antibody comprising a HC variable region comprising or consisting of SEQ ID No. 292 or a sequence with at least 80% sequence identity thereto and a LC variable region comprising or consisting of SEQ ID No. 294 or a sequence with at least 80% sequence identity thereto;
an antibody comprising a HC variable region comprising or consisting of SEQ ID No. 302 or a sequence with at least 80% sequence identity thereto and a LC variable region comprising or consisting of SEQ ID No. 304 or a sequence with at least 80% sequence identity thereto;
an antibody comprising a HC variable region comprising or consisting of SEQ ID No. 312 or a sequence with at least 80% sequence identity thereto and a LC variable region comprising or consisting of SEQ ID No. 314 or a sequence with at least 80% sequence identity thereto;
an antibody comprising a HC variable region comprising or consisting of SEQ ID No. 322 or a sequence with at least 80% sequence identity thereto and a LC variable region comprising or consisting of SEQ ID No. 324 or a sequence with at least 80% sequence identity thereto;
an antibody comprising a HC variable region comprising or consisting of SEQ ID No. 332 or a sequence with at least 80% sequence identity thereto and a LC variable region comprising or consisting of SEQ ID No. 334 or a sequence with at least 80% sequence identity thereto;
an antibody comprising a HC variable region comprising or consisting of SEQ ID No. 342 or a sequence with at least 80% sequence identity thereto and a LC variable region comprising or consisting of SEQ ID No. 344 or a sequence with at least 80% sequence identity thereto;
an antibody comprising a HC variable region comprising or consisting of SEQ ID No. 352 or a sequence with at least 80% sequence identity thereto and a LC variable region comprising or consisting of SEQ ID No. 354 or a sequence with at least 80% sequence identity thereto;
an antibody comprising a HC variable region comprising or consisting of SEQ ID No. 362 or a sequence with at least 80% sequence identity thereto and a LC variable region comprising or consisting of SEQ ID No. 364 or a sequence with at least 80% sequence identity thereto;
an antibody comprising a HC variable region comprising or consisting of SEQ ID No. 372 or a sequence with at least 80% sequence identity thereto and a LC variable region comprising or consisting of SEQ ID No. 374 or a sequence with at least 80% sequence identity thereto
an antibody comprising a HC variable region comprising or consisting of SEQ ID No. 382 or a sequence with at least 80% sequence identity thereto and a LC variable region comprising or consisting of SEQ ID No. 384 or a sequence with at least 80% sequence identity thereto;
an antibody comprising a HC variable region comprising or consisting of SEQ ID No. 392 or a sequence with at least 80% sequence identity thereto and a LC variable region comprising or consisting of SEQ ID No. 394 or a sequence with at least 80% sequence identity thereto;
an antibody comprising a HC variable region comprising or consisting of SEQ ID No. 402 or a sequence with at least 80% sequence identity thereto and a LC variable region comprising or consisting of SEQ ID No. 404 or a sequence with at least 80% sequence identity thereto;
an antibody comprising a HC variable region comprising or consisting of SEQ ID No. 412 or a sequence with at least 80% sequence identity thereto and a LC variable region comprising or consisting of SEQ ID No. 414 or a sequence with at least 80% sequence identity thereto;
an antibody comprising a HC variable region comprising or consisting of SEQ ID No. 422 or a sequence with at least 80% sequence identity thereto and a LC variable region comprising or consisting of SEQ ID No. 424 or a sequence with at least 80% sequence identity thereto;
an antibody comprising a HC variable region comprising or consisting of SEQ ID No. 432 or a sequence with at least 80% sequence identity thereto and a LC variable region comprising or consisting of SEQ ID No. 434 or a sequence with at least 80% sequence identity thereto;
an antibody comprising a HC variable region comprising or consisting of SEQ ID No. 442 or a sequence with at least 80% sequence identity thereto and a LC variable region comprising or consisting of SEQ ID No. 444 or a sequence with at least 80% sequence identity thereto;
an antibody comprising a HC variable region comprising or consisting of SEQ ID No. 452 or a sequence with at least 80% sequence identity thereto and a LC variable region comprising or consisting of SEQ ID No. 454 or a sequence with at least 80% sequence identity thereto;
an antibody comprising a HC variable region comprising or consisting of SEQ ID No. 462 or a sequence with at least 80% sequence identity thereto and a LC variable region comprising or consisting of SEQ ID No. 464 or a sequence with at least 80% sequence identity thereto;
an antibody comprising a HC variable region comprising or consisting of SEQ ID No. 472 or a sequence with at least 80% sequence identity thereto and a LC variable region comprising or consisting of SEQ ID No. 474 or a sequence with at least 80% sequence identity thereto;
an antibody comprising a HC variable region comprising or consisting of SEQ ID No. 482 or a sequence with at least 80% sequence identity thereto and a LC variable region comprising or consisting of SEQ ID No. 484 or a sequence with at least 80% sequence identity thereto;
an antibody comprising a HC variable region comprising or consisting of SEQ ID No. 492 or a sequence with at least 80% sequence identity thereto and a LC variable region comprising or consisting of SEQ ID No. 494 or a sequence with at least 80% sequence identity thereto;
an antibody comprising a HC variable region comprising or consisting of SEQ ID No. 502 or a sequence with at least 80% sequence identity thereto and a LC variable region comprising or consisting of SEQ ID No. 504 or a sequence with at least 80% sequence identity thereto;
an antibody comprising a HC variable region comprising or consisting of SEQ ID No. 512 or a sequence with at least 80% sequence identity thereto and a LC variable region comprising or consisting of SEQ ID No. 514 or a sequence with at least 80% sequence identity thereto;
an antibody comprising a HC variable region comprising or consisting of SEQ ID No. 522 or a sequence with at least 80% sequence identity thereto and a LC variable region comprising or consisting of SEQ ID No. 524 or a sequence with at least 80% sequence identity thereto;
an antibody comprising a HC variable region comprising or consisting of SEQ ID No. 532 or a sequence with at least 80% sequence identity thereto and a LC variable region comprising or consisting of SEQ ID No. 534 or a sequence with at least 80% sequence identity thereto;
an antibody comprising a HC variable region comprising or consisting of SEQ ID No. 542 or a sequence with at least 80% sequence identity thereto and a LC variable region comprising or consisting of SEQ ID No. 544 or a sequence with at least 80% sequence identity thereto;
an antibody comprising a HC variable region comprising or consisting of SEQ ID No. 552 or a sequence with at least 80% sequence identity thereto and a LC variable region comprising or consisting of SEQ ID No. 554 or a sequence with at least 80% sequence identity thereto;
an antibody comprising a HC variable region comprising or consisting of SEQ ID No. 562 or a sequence with at least 80% sequence identity thereto and a LC variable region comprising or consisting of SEQ ID No. 564 or a sequence with at least 80% sequence identity thereto;
an antibody comprising a HC variable region comprising or consisting of SEQ ID No. 572 or a sequence with at least 80% sequence identity thereto and a LC variable region comprising or consisting of SEQ ID No. 574 or a sequence with at least 80% sequence identity thereto;
an antibody comprising a HC variable region comprising or consisting of SEQ ID No. 582 or a sequence with at least 80% sequence identity thereto and a LC variable region comprising or consisting of SEQ ID No. 584 or a sequence with at least 80% sequence identity thereto;
an antibody comprising a HC variable region comprising or consisting of SEQ ID No. 592 or a sequence with at least 80% sequence identity thereto and a LC variable region comprising or consisting of SEQ ID No. 594 or a sequence with at least 80% sequence identity thereto;
an antibody comprising a HC variable region comprising or consisting of SEQ ID No. 602 or a sequence with at least 80% sequence identity thereto and a LC variable region comprising or consisting of SEQ ID No. 604 or a sequence with at least 80% sequence identity thereto;
an antibody comprising a HC variable region comprising or consisting of SEQ ID No. 612 or a sequence with at least 80% sequence identity thereto and a LC variable region comprising or consisting of SEQ ID No. 614 or a sequence with at least 80% sequence identity thereto;
an antibody comprising a HC variable region comprising or consisting of SEQ ID No. 622 or a sequence with at least 80% sequence identity thereto and a LC variable region comprising or consisting of SEQ ID No. 624 or a sequence with at least 80% sequence identity thereto;
an antibody comprising a HC variable region comprising or consisting of SEQ ID No. 632 or a sequence with at least 80% sequence identity thereto and a LC variable region comprising or consisting of SEQ ID No. 634 or a sequence with at least 80% sequence identity thereto;
an antibody comprising a HC variable region comprising or consisting of SEQ ID No. 642 or a sequence with at least 80% sequence identity thereto and a LC variable region comprising or consisting of SEQ ID No. 644 or a sequence with at least 80% sequence identity thereto;
an antibody comprising a HC variable region comprising or consisting of SEQ ID No. 652 or a sequence with at least 80% sequence identity thereto and a LC variable region comprising or consisting of SEQ ID No. 654 or a sequence with at least 80% sequence identity thereto;
an antibody comprising a HC variable region comprising or consisting of SEQ ID No. 662 or a sequence with at least 80% sequence identity thereto and a LC variable region comprising or consisting of SEQ ID No. 664 or a sequence with at least 80% sequence identity thereto;
an antibody comprising a HC variable region comprising or consisting of SEQ ID No. 672 or a sequence with at least 80% sequence identity thereto and a LC variable region comprising or consisting of SEQ ID No. 674 or a sequence with at least 80% sequence identity thereto;
an antibody comprising a HC variable region comprising or consisting of SEQ ID No. 682 or a sequence with at least 80% sequence identity thereto and a LC variable region comprising or consisting of SEQ ID No. 684 or a sequence with at least 80% sequence identity thereto;
an antibody comprising a HC variable region comprising or consisting of SEQ ID No. 692 or a sequence with at least 80% sequence identity thereto and a LC variable region comprising or consisting of SEQ ID No. 694 or a sequence with at least 80% sequence identity thereto;
an antibody comprising a HC variable region comprising or consisting of SEQ ID No. 702 or a sequence with at least 80% sequence identity thereto and a LC variable region comprising or consisting of SEQ ID No. 704 or a sequence with at least 80% sequence identity thereto;
an antibody comprising a HC variable region comprising or consisting of SEQ ID No. 712 or a sequence with at least 80% sequence identity thereto and a LC variable region comprising or consisting of SEQ ID No. 714 or a sequence with at least 80% sequence identity thereto; or
an antibody comprising a HC variable region comprising or consisting of SEQ ID No. 738 or a sequence with at least 80% sequence identity thereto and a LC variable region comprising or consisting of SEQ ID No. 740 or a sequence with at least 80% sequence identity thereto;
an antibody comprising a HC variable region comprising or consisting of SEQ ID No. 748 or a sequence with at least 80% sequence identity thereto and a LC variable region comprising or consisting of SEQ ID No. 750 or a sequence with at least 80% sequence identity thereto;
an antibody comprising a HC variable region comprising or consisting of SEQ ID No. 758 or a sequence with at least 80% sequence identity thereto and a LC variable region comprising or consisting of SEQ ID No. 760 or a sequence with at least 80% sequence identity thereto;
an antibody comprising a HC variable region comprising or consisting of SEQ ID No. 768 or a sequence with at least 80% sequence identity thereto and a LC variable region comprising or consisting of SEQ ID No. 770 or a sequence with at least 80% sequence identity thereto;
an antibody comprising a HC variable region comprising or consisting of SEQ ID No. 778 or a sequence with at least 80% sequence identity thereto and a LC variable region comprising or consisting of SEQ ID No. 780 or a sequence with at least 80% sequence identity thereto;
an antibody comprising a HC variable region comprising or consisting of SEQ ID No. 788 or a sequence with at least 80% sequence identity thereto and a LC variable region comprising or consisting of SEQ ID No. 790 or a sequence with at least 80% sequence identity thereto, or;
an antibody comprising a HC variable region comprising or consisting of SEQ ID No. 798 or a sequence with at least 80% sequence identity thereto and a LC variable region comprising or consisting of SEQ ID No. 800 or a sequence with at least 80% sequence identity thereto.

8. The antibody or fragment according to claim 2 wherein said fragment is selected from a F(ab′)2, Fab, Fv, scFv, heavy chain, light chain, variable heavy (VH), variable light (VL) chain, CDR region, single VH or VL domain, maxibodies, minibodies, intrabodies, diabodies, triabodies, tetrabodies, and bis-scFv, and polypeptides that contain at least a portion of an immunoglobulin that is sufficient to confer specific antigen binding to the polypeptide.

9. The antibody or fragment according to claim 8 wherein said antibody or fragment is conjugated to another moiety.

10. The antibody or fragment according to claim 9 further comprising a therapeutic moiety, half-life extending moiety or label.

11. (canceled)

12. (canceled)

13. A pharmaceutical composition comprising an antibody or fragment thereof according to claim 2.

14. (canceled)

15. A method of treating or preventing an OX40 or OX40L-mediated disease comprising administering to a subject in need thereof the pharmaceutical composition according to claim 13.

16. The method of claim 15 wherein said disease is selected from an inflammatory or autoimmune disease.

17. The method according claim 16 wherein said disease is selected from an inflammatory skin diseases, including atopic dermatitis, allergic dermatitis, pruritus, psoriasis, scleroderma, or eczema; responses associated with inflammatory bowel disease (such as Crohn's disease and ulcerative colitis); ischemic reperfusion; adult respiratory distress syndrome; asthma; meningitis; encephalitis; uveitis; autoimmune diseases such as rheumatoid arthritis, Sjorgen's syndrome, vasculitis; diseases involving leukocyte diapedesis; central nervous system (CNS) inflammatory disorder, multiple organ injury syndrome secondary to septicaemia or trauma, bacterial pneumonia, antigen-antibody complex mediated diseases; inflammations of the lung, including pleurisy, alveolitis, vasculitis, pneumonia, chronic bronchitis, bronchiectasis, and cystic fibrosis.

18. The method according to claim 17 wherein said antibody or fragment is administered together with one or more therapeutic agent.

19. The method according claim 18 wherein said one or more therapeutic agent is selected from rapamycin (sirolimus), tacrolimus, cyclosporine (e.g. Atopica®), corticosteroids (e.g. methylprednisolone), methotrexate, mycophenolate mofetil, anti-CD28 antibodies, anti-IL 12/IL-23 antibodies, antiCD20 antibodies, anti-CD30 antibodies, CTLA4-Fc molecules, CCRS receptor antagonists, antiCD40L antibodies, anti-VI_A4 antibodies, anti-LFA1 antibodies, fludarabine, anti-CD52 antibodies, anti-CD45 antibodies, cyclophosphamide, anti-thymocyte globulins, anticomplement CS antibodies, anti-a4b7 integrin antibodies, anti-IL6 antibodies, anti-IL6-R antibodies, anti-IL2R antibodies, anti-CD25 antibodies, anti-TNFa/TNFa-Fc molecules, HDAC inhibitors, JAK inhibitors, such as JAK-1 and JAK-3 inhibitors, anti-IL-31 antibodies, SYK inhibitors, anti-IL-4Ra antibodies, anti-IL-13 antibodies, anti-TSLP antibodies, PDE4 inhibitors, lokietmab (Cytopoint®), and oclacitinib (Apoquel®).

20. A method of decreasing the secretion of cytokines comprising administering to a subject in need thereof the pharmaceutical composition of claim 13.

21. (canceled)

22. (canceled)

23. (canceled)

24. A kit comprising the pharmaceutical composition according to claim 13.

25. A method for detecting OX40L or OX40 in a companion animal comprising contacting a test sample with an antibody or fragment according to claim 2.


SEQUENCE LISTING (811 sequences)

SEQ ID NO: 1 - protein, 183 aa, Canis sp.
MEGVQPLDQNVGNTPGRRFQKNKVLLVAAIIQGLGLLLCFTYICLHFYASQVPPQYPPIQSIRVQFTRCENEKGCIITSPSKDETMKVQDNSIIINCDGFYLISLKGYFSEELSLSLYYRKGRGPLFSLSKVTSVDSIGVAYLAFKDKVYFNVTTHSTSYKDIQVNGGELILIHQNPGGFCAY

SEQ ID NO: 2 - DNA, 552 bp, Canis sp.
  source 1..552: mol_type = genomic DNA
atggaaggggtccaacccctggaccagaatgtgggaaacacaccagggcgaagattccagaagaacaaggtattgctggtggcagccataattcaggggctgggtctgctcctgtgtttcacctacatctgcctgcacttctatgcttctcaggtgccgcctcagtatcctccaattcaaagtatcagagtacaatttaccaggtgtgagaatgagaaaggttgcatcatcacatccccaagcaaggatgaaactatgaaggtgcaagacaactcaatcatcattaactgtgatgggttttatctcatctccctgaagggttacttctctgaggagctcagcctcagcctttattaccgaaagggtcggggacccctcttctctctgagcaaggtcacatctgttgactccattggagtggcctatctggctttcaaggacaaagtctactttaatgtgaccactcacagtacctcctacaaagacatccaggtgaatggtggggaattgattctcattcatcaaaatcctggtggcttctgtgcctactga

SEQ ID NO: 3 - DNA, 708 bp, Canis sp.
  source 1..708: mol_type = genomic DNA
atgaggatgttcgtggagtccctgcggctcagcggtcctcactcagccctcctgctcctggggcttgtgctgggtgccgtagctgagcacaactgtttcgggaacacctaccccaaagacggcaagtgctgcaatgactgcccaccaggttatggaatggagagccgctgcagtaggagccatgacaccaaatgtcatcagtgtccatctggcttctacaatgaggctacaaattacgaaccctgcaagccctgcactcagtgcaatcagagaagtgggagtgaacccaagaggagatgcacacccacgcaggacaccatctgcagctgtaagccaggcacagagccccgggacggctacaagcgtggagtcgactgtgccccatgcccacccggacacttctccccaggggatgaccaggcctgcaagccctggaccaactgtaccttgatgggaaggcgtacaatgcagccggccagcaagagctcagacgctgtctgtgaggacaggagcctccccgccacactgccatgggagacccagagccccctgacccggccccctacccctcagcccactatggcctggcccaggacctcgcaggggcccttcacaccccctacggagccccccaggggtggaaatagcttccggacccccatccaagaggagcatgctgacgccaactccaccctggccaagatctga

SEQ ID NO: 4 - protein, 235 aa, Canis sp.
MRMFVESLRLSGPHSALLLLGLVLGAVAEHNCFGNTYPKDGKCCNDCPPGYGMESRCSRSHDTKCHQCPSGFYNEATNYEPCKPCTQCNQRSGSEPKRRCTPTQDTICSCKPGTEPRDGYKRGVDCAPCPPGHFSPGDDQACKPWTNCTLMGRRTMQPASKSSDAVCEDRSLPATLPWETQSPLTRPPTPQPTMAWPRTSQGPFTPPTEPPRGGNSFRTPIQEEHADANSTLAKI

SEQ ID NO: 5 - DNA, 813 bp, Canis sp.
  source 1..813: mol_type = genomic DNA
atgaggatgttcgtggagtccctgcggctcagcggtcctcactcagccctcctgctcctggggcttgtgctgggtgccgtagctgagcacaactgtttcgggaacacctaccccaaagacggcaagtgctgcaatgactgcccaccaggttatggaatggagagccgctgcagtaggagccatgacaccaaatgtcatcagtgtccatctggcttctacaatgaggctacaaattacgaaccctgcaagccctgcactcagtgcaatcagagaagtgggagtgaacccaagaggagatgcacacccacgcaggacaccatctgcagctgtaagccaggcacagagccccgggacggctacaagcgtggagtcgactgtgccccatgcccacccggacacttctccccaggggatgaccaggcctgcaagccctggaccaactgtaccttgatgggaaggcgtacaatgcagccggccagcaagagctcagacgctgtctgtgaggacaggagcctccccgccacactgccatgggagacccagagccccctgacccggccccctacccctcagcccactatggcctggcccaggacctcgcaggggcccttcacaccccctacggagccccccaggggcccccagctggctgctgtcctgggcttgggcctaggcttgctggcccccgtggcagccgcactggccttgctcctgcaccacagagcctggcggctgccccccggtggaaatagcttccggacccccatccaagaggagcatgctgacgccaactccaccctggccaagatctga

SEQ ID NO: 6 - protein, 270 aa, Canis sp.
MRMFVESLRLSGPHSALLLLGLVLGAVAEHNCFGNTYPKDGKCCNDCPPGYGMESRCSRSHDTKCHQCPSGFYNEATNYEPCKPCTQCNQRSGSEPKRRCTPTQDTICSCKPGTEPRDGYKRGVDCAPCPPGHFSPGDDQACKPWTNCTLMGRRTMQPASKSSDAVCEDRSLPATLPWETQSPLTRPPTPQPTMAWPRTSQGPFTPPTEPPRGPQLAAVLGLGLGLLAPVAAALALLLHHRAWRLPPGGNSFRTPIQEEHADANSTLAKI

SEQ ID NO: 7 - DNA, 20 bp, synthetic construct
  source 1..20: mol_type = other DNA
ggcagagatgaggatgttcg

SEQ ID NO: 8 - DNA, 21 bp, synthetic construct
  source 1..21: mol_type = other DNA
cactggctgctcagatcttgg

SEQ ID NO: 9 - DNA, 20 bp, synthetic construct
  source 1..20: mol_type = other DNA
gccacagttttcatctccct

SEQ ID NO: 10 - DNA, 18 bp, synthetic construct
  source 1..18: mol_type = other DNA
gcttggcttaggtgcagc

SEQ ID NO: 11 - DNA, 358 bp, Canis sp.
  source 1..358: mol_type = genomic DNA
gaggtgcacctggtggagtctgggggagacctggtgaagcctggggggtccctgagactttcctgtgtggcctctggattcaccttcagtagctaccacatgagttgggtccgccaggctccagggaagggacttcagtgggtcgcattcattaacgctggtgaaattagcacaaactatgcagacgctgtgaagggccgattcaccatctccagagacaacgccaagaatatattgtatcttcagatgaacagcctgagagccgaggacacggccgtgtattactgtgcgagtggacatatttggatacgtccctttgactactggggccagggaaccctggtcaccgtctcctcag

SEQ ID NO: 12 - protein, 119 aa, Canis sp.
EVHLVESGGDLVKPGGSLRLSCVASGFTFSSYHMSWVRQAPGKGLQWVAFINAGEISTNYADAVKGRFTISRDNAKNILYLQMNSLRAEDTAVYYCASGHIWIRPFDYWGQGTLVTVSS

SEQ ID NO: 13 - DNA, 322 bp, Canis sp.
  source 1..322: mol_type = other DNA
tcctatgtgctgacacagctgccatccatgagtgtgaccctgaggcagacggcccgcatcacctgtgagggagacagcattggaaggaaaagagttaactggtaccagcagaagttgggccaggtccctgtactaattatttatgatgatgacagcaggccgtcagggatccctgagcgattctccggcgccaactcggggaacacagccaccctgaccatcagcggggccctggccgaggacgaggctgaatattactgccaggtgtgggacagcagtaataattgggtattcggtgaggggacccagctgaccgtcctcg

SEQ ID NO: 14 - protein, 107 aa, Canis sp.
SYVLTQLPSMSVTLRQTARITCEGDSIGRKRVNWYQQKLGQVPVLIIYDDDSRPSGIPERFSGANSGNTATLTISGALAEDEAEYYCQVWDSSNNWVFGEGTQLTVL

SEQ ID NO: 15 - protein, 7 aa, Canis sp.
GFTFSSY

SEQ ID NO: 16 - protein, 6 aa, Canis sp.
NAGEIS

SEQ ID NO: 17 - protein, 14 aa, Canis sp.
CASGHIWIRPFDYW

SEQ ID NO: 18 - protein, 11 aa, Canis sp.
EGDSIGRKRVN

SEQ ID NO: 19 - protein, 7 aa, Canis sp.
DDDSRPS

SEQ ID NO: 20 - protein, 12 aa, Canis sp.
CQVWDSSNNWVF

SEQ ID NO: 21 - DNA, 358 bp, Canis sp.; identical to SEQ ID NO: 11
  source 1..358: mol_type = other DNA

SEQ ID NO: 22 - protein, 119 aa, Canis sp.; identical to SEQ ID NO: 12

SEQ ID NO: 23 - DNA, 322 bp, Canis sp.; identical to SEQ ID NO: 13
  source 1..322: mol_type = other DNA

SEQ ID NO: 24 - protein, 107 aa, Canis sp.; identical to SEQ ID NO: 14

SEQ ID NO: 25 - protein, 7 aa, Canis sp.
GFTFSSY

SEQ ID NO: 26 - protein, 6 aa, Canis sp.
NAGEIS

SEQ ID NO: 27 - protein, 14 aa, Canis sp.
CASGHIWIRPFDYW

SEQ ID NO: 28 - protein, 11 aa, Canis sp.
EGDSIGRKRVN

SEQ ID NO: 29 - protein, 7 aa, Canis sp.
DDDSRPS

SEQ ID NO: 30 - protein, 12 aa, Canis sp.
CQVWDSSNNWVF

SEQ ID NO: 31 - DNA, 358 bp, Canis sp.
  source 1..358: mol_type = other DNA
gaggtgcacctggtggagtctgggggagacctggtgaagcctggggggtccctgagactttcctgtgtggcctctggattcaccttcagtacctaccacatgagttgggtccgccaggctccagggaagggacttcagtgggtcgcattcattaacgctggtgaaattagcacaaactatgcagacgctgtgaagggccgattcaccatctccagagacaacgccaagaatatattctatcttcagatgaacagcctgagagccgaggacacggccgtgtattactgtgcgactggacatatttggatacgtccctttgactactggggccagggaaccctggtcaccgtctcctcag

SEQ ID NO: 32 - protein, 119 aa, Canis sp.
EVHLVESGGDLVKPGGSLRLSCVASGFTFSTYHMSWVRQAPGKGLQWVAFINAGEISTNYADAVKGRFTISRDNAKNIFYLQMNSLRAEDTAVYYCATGHIWIRPFDYWGQGTLVTVSS

SEQ ID NO: 33 - DNA, 322 bp, Canis sp.
  source 1..322: mol_type = other DNA
tcctatgtactgacacagctgccatccatgagtgtgaccctgaggcagacggcccgcatcacctgtgagggaaacagcattggaaggaaaagagttaactggtaccagcagaagttgggccaggtccctgtactaattatttatgatgatgacagcaggccgtcagggatccctgagcgattctccggcgccaactcggggaacacagccaccctgaccatcagcggggccctggccgaggacgaggctgaatattactgccaggtgtgggacagcagtaataattgggtattcggtgaggggacccagctgaccgtcctcg

SEQ ID NO: 34 - protein, 107 aa, Canis sp.
SYVLTQLPSMSVTLRQTARITCEGNSIGRKRVNWYQQKLGQVPVLIIYDDDSRPSGIPERFSGANSGNTATLTISGALAEDEAEYYCQVWDSSNNWVFGEGTQLTVL

SEQ ID NO: 35 - protein, 7 aa, Canis sp.
GFTFSTY

SEQ ID NO: 36 - protein, 6 aa, Canis sp.
NAGEIS

SEQ ID NO: 37 - protein, 14 aa, Canis sp.
CATGHIWIRPFDYW

SEQ ID NO: 38 - protein, 11 aa, Canis sp.
EGNSIGRKRVN

SEQ ID NO: 39 - protein, 7 aa, Canis sp.
DDDSRPS

SEQ ID NO: 40 - protein, 12 aa, Canis sp.
CQVWDSSNNWVF

SEQ ID NO: 41 - DNA, 361 bp, Canis sp.
  source 1..361: mol_type = other DNA
gaggtgcagatggtggagtctgggggagacctggtgaagcctggggggtccctgagactttcctgtgtggcctctggattcaccttcagtgactaccacatgagctgggtccgccaggctccagggaagggacttcagtgggtcgcatacattaccagtggtggaagtagcacaagctatgcagacgctgtgaagggccgattcaccatctccagagacaacgccaagaacacgctgtatcttcagatgaacagcctgagagccgaggacacggccgtgtattattgtgcgagtgatctagttctagtagttggcgggggggtctactggggccagggaaccctggtcaccgtctcctcag

SEQ ID NO: 42 - protein, 120 aa, Canis sp.
EVQMVESGGDLVKPGGSLRLSCVASGFTFSDYHMSWVRQAPGKGLQWVAYITSGGSSTSYADAVKGRFTISRDNAKNTLYLQMNSLRAEDTAVYYCASDLVLVVGGGVYWGQGTLVTVSS

SEQ ID NO: 43 - DNA, 322 bp, Canis sp.
  source 1..322: mol_type = other DNA
tcctatgaactgactcagccaccatccgtgaatgtgaccctgagggagacggcccacatcacctgtgggggagacagcattggaagtaaatatgttcaatggatccagcagaatccaggccaggcccccgtggtgattatctataaagatagcaacaggccgacagggatccctgagcgattctctggcgccaactcagggaacacggctaccctgaccatcagtggggccctggccgaagacgaggctgactattactgccaggtgggggacagtggtactaaggcggtattcggtgaagggacccagctgaccgtcctcg

SEQ ID NO: 44 - protein, 107 aa, Canis sp.
SYELTQPPSVNVTLRETAHITCGGDSIGSKYVQWIQQNPGQAPVVIIYKDSNRPTGIPERFSGANSGNTATLTISGALAEDEADYYCQVGDSGTKAVFGEGTQLTVL

SEQ ID NO: 45 - protein, 7 aa, Canis sp.
GFTFSDY

SEQ ID NO: 46 - protein, 6 aa, Canis sp.
TSGGSS

SEQ ID NO: 47 - protein, 15 aa, Canis sp.
CASDLVLVVGGGVYW

SEQ ID NO: 48 - protein, 11 aa, Canis sp.
GGDSIGSKYVQ

SEQ ID NO: 49 - protein, 7 aa, Canis sp.
KDSNRPT

SEQ ID NO: 50 - protein, 12 aa, Canis sp.
CQVGDSGTKAVF

SEQ ID NO: 51 - DNA, 352 bp, Canis sp.
  source 1..352: mol_type = other DNA
gaggtgcagctggtggagtctgggggagacctggtgaagcctggggggtccctgagactttcctgtgtggcctctggattcaccttcagttcctaccacatgaactgggtccgccaggctccagggaaggggcttcagtgggtcgcatacattaacactggtggaagtcgcacaagctatgcagacgctgtgaagggccgattcaccatctccagagacaacgccaagaacacgttgtatcttcagatgaacagcctgagagccgaggacacggccgtgtattactgtgcgaataactactgggataattttgactcctggggccagggaaccctggtcaccgtctcctcag

SEQ ID NO: 52 - protein, 117 aa, Canis sp.
EVQLVESGGDLVKPGGSLRLSCVASGFTFSSYHMNWVRQAPGKGLQWVAYINTGGSRTSYADAVKGRFTISRDNAKNTLYLQMNSLRAEDTAVYYCANNYWDNFDSWGQGTLVTVSS

SEQ ID NO: 53 - DNA, 325 bp, Canis sp.
  source 1..325: mol_type = other DNA
tcctatgagctgactcagccaccatccgtgaatgtgaccctgagggagacggcccacatcacctgtgggggagacagcattggaagtaaatatgttcaatggatccagcagaatccaggccaggcccccgtggtgattatctataaagataccaacaggccgacagggatccctgagcgattctctggcgccaactcagggaacacggctaccctgaccatcagtggggccctggccgaagacgaggctgactattactgccaggtgggggacagtggtactaaggctagtgtgttcggcggaggcacccatctgaccgtcctcg

SEQ ID NO: 54 - protein, 108 aa, Canis sp.
SYELTQPPSVNVTLRETAHITCGGDSIGSKYVQWIQQNPGQAPVVIIYKDTNRPTGIPERFSGANSGNTATLTISGALAEDEADYYCQVGDSGTKASVFGGGTHLTVL

SEQ ID NO: 55 - protein, 7 aa, Canis sp.
GFTFSSY

SEQ ID NO: 56 - protein, 6 aa, Canis sp.
NTGGSR

SEQ ID NO: 57 - protein, 12 aa, Canis sp.
CANNYWDNFDSW

SEQ ID NO: 58 - protein, 11 aa, Canis sp.
GGDSIGSKYVQ

SEQ ID NO: 59 - protein, 7 aa, Canis sp.
KDTNRPT

SEQ ID NO: 60 - protein, 13 aa, Canis sp.
CQVGDSGTKASVF

SEQ ID NO: 61 - DNA, 361 bp, Canis sp.
  source 1..361: mol_type = other DNA
gaggtgcagctggtggagtctgggggagacctggtgaagcctggggggtccctgagactttcctgtgtggcctctggattcaccttcagtaactaccacatgaactgggtccgccaggctccagggaagggacttcagtgggtcgcatacgttgtcagtggtggaagtaccacaagctatgcagacgctgtgaagggccgattcaccatctccagagacaacgccaagaacacactgtatcttcagatgaacagcctgagagccgaggacacggccgtgtattactgtgcgaaggggactagtagttggggggttcctatggactactggggccatggcacctcactcttcgtgtcctcag

SEQ ID NO: 62 - protein, 120 aa, Canis sp.
EVQLVESGGDLVKPGGSLRLSCVASGFTFSNYHMNWVRQAPGKGLQWVAYVVSGGSTTSYADAVKGRFTISRDNAKNTLYLQMNSLRAEDTAVYYCAKGTSSWGVPMDYWGHGTSLFVSS

SEQ ID NO: 63 - DNA, 319 bp, Canis sp.
  source 1..319: mol_type = other DNA
tcctatgtgctgacacagctgccatccgtgagtgtgaccctgaggcagacggcccgcatcacctgtgggggagacagcattagaagtaaaaatgtttactggtaccagcagaagctgggccaggcccctgtactgattatctatgatgatagcagcaggccgtcagggatccctgagcgattctccggcgccaactcggggaacacggccaccctgaccatcagcggggccctggccgaggatgaggctgactattactgccaggtgtgggacagcagtactagtgtgttcggcggaggcacccatctgaccgtcctcg

SEQ ID NO: 64 - protein, 106 aa, Canis sp.
SYVLTQLPSVSVTLRQTARITCGGDSIRSKNVYWYQQKLGQAPVLIIYDDSSRPSGIPERFSGANSGNTATLTISGALAEDEADYYCQVWDSSTSVFGGGTHLTVL

SEQ ID NO: 65 - protein, 7 aa, Canis sp.
GFTFSNY

SEQ ID NO: 66 - protein, 6 aa, Canis sp.
VSGGST

SEQ ID NO: 67 - protein, 15 aa, Canis sp.
CAKGTSSWGVPMDYW

SEQ ID NO: 68 - protein, 11 aa, Canis sp.
GGDSIRSKNVY

SEQ ID NO: 69 - protein, 7 aa, Canis sp.
DDSSRPS

SEQ ID NO: 70 - protein, 11 aa, Canis sp.
CQVWDSSTSVF

SEQ ID NO: 71 - DNA, 361 bp, Canis sp.
  source 1..361: mol_type = other DNA
gaggtgcagctggtggagtctgggggagacctggtgaagcctggggggtccctgagactttcctgtgtggcctctggattcaccttcagtaactaccacatgaactgggtccgccaggctccagggaagggacttcagtgggtcgcatacgttgtcagtggtggaactaccacgacctatgcagacgctgtgaagggccgattcaccatctccagagacaacgccaagaacacactgtatcttcagatgaacagcctgagagccgaggacacggccgtgtattactgtgcgaaggggactagtacttggggggttcctatggactactggggccatggcacctcactcttcgtgtcctcag

SEQ ID NO: 72 - protein, 120 aa, Canis sp.
EVQLVESGGDLVKPGGSLRLSCVASGFTFSNYHMNWVRQAPGKGLQWVAYVVSGGTTTTYADAVKGRFTISRDNAKNTLYLQMNSLRAEDTAVYYCAKGTSTWGVPMDYWGHGTSLFVSS

SEQ ID NO: 73 - DNA, 319 bp, Canis sp.
  source 1..319: mol_type = other DNA
tcctatgtgctgacacagctgccatccgtgagtgtgaccctgaggcagacggcccgcatcacctgtgggggagacagcattagaagtaaaaatgtttactggtaccagaagaagctgggccaggcccctgtactgattatctatgatgatagcagcaggccgtcagggatccctgagcgattctccggcgccaactcggggaacacggccaccctgaccatcagcggggccctggccgaggatgaggctgactattactgccaggtgtgggacagcagtactagtgtgttcggcggaggcacccatctgaccgtcctcg

SEQ ID NO: 74 - protein, 106 aa, Canis sp.
SYVLTQLPSVSVTLRQTARITCGGDSIRSKNVYWYQKKLGQAPVLIIYDDSSRPSGIPERFSGANSGNTATLTISGALAEDEADYYCQVWDSSTSVFGGGTHLTVL

SEQ ID NO: 75 - protein, 7 aa, Canis sp.
GFTFSNY

SEQ ID NO: 76 - protein, 6 aa, Canis sp.
VSGGTT

SEQ ID NO: 77 - protein, 15 aa, Canis sp.
CAKGTSTWGVPMDYW

SEQ ID NO: 78 - protein, 11 aa, Canis sp.
GGDSIRSKNVY

SEQ ID NO: 79 - protein, 7 aa, Canis sp.
DDSSRPS

SEQ ID NO: 80 - protein, 11 aa, Canis sp.
CQVWDSSTSVF

SEQ ID NO: 81 - DNA, 361 bp, Canis sp.
  source 1..361: mol_type = other DNA
gaggtgcagctggtggagtctgggggagacctggtgaagccgggggggtccctgagactttcctgtgtggcctctggattcaccttcagcagttatcacatgagctgggtccgccaggctccagggaaggggcttcagtgggtcgcatacattaccagtggtggaagaaccacaagctatgcagacgctgtgaagggccgattcatcatctccagagacaacgccaagaacacgttgtatcttcagatgaacagcctgagagccgaggacacggccgtgtattactgtgcgagtgatctggttctaatattagggggggctgactactggggccagggaaccctggtcaccgtctcctcag

SEQ ID NO: 82 - protein, 120 aa, Canis sp.
EVQLVESGGDLVKPGGSLRLSCVASGFTFSSYHMSWVRQAPGKGLQWVAYITSGGRTTSYADAVKGRFIISRDNAKNTLYLQMNSLRAEDTAVYYCASDLVLILGGADYWGQGTLVTVSS

SEQ ID NO: 83 - DNA, 322 bp, Canis sp.
  source 1..322: mol_type = other DNA
tcctatgagctgactcagccaccatccgtgaatgtgaccctgagggagacggcccacatcacctgtgggggagacagcattggaaggaaatatgttcaatggatccagcagaatccaggccaggcccccgtggtgattatctataaagataacaacaggccgacagggatccctgagcgattctctggcgccaactcagggaacacggctaccctgaccatcagtggggccctggccgaagacgaggctgactattactgccaggtgggggacagtggttctaaggctgtgttcggcggaggcacccacctgaccgtcctcg

SEQ ID NO: 84 - protein, 107 aa, Canis sp.
SYELTQPPSVNVTLRETAHITCGGDSIGRKYVQWIQQNPGQAPVVIIYKDNNRPTGIPERFSGANSGNTATLTISGALAEDEADYYCQVGDSGSKAVFGGGTHLTVL

SEQ ID NO: 85 - protein, 7 aa, Canis sp.
GFTFSSY

SEQ ID NO: 86 - protein, 6 aa, Canis sp.
TSGGRT

SEQ ID NO: 87 - protein, 15 aa, Canis sp.
CASDLVLILGGADYW

SEQ ID NO: 88 - protein, 11 aa, Canis sp.
GGDSIGRKYVQ

SEQ ID NO: 89 - protein, 7 aa, Canis sp.
KDNNRPT

SEQ ID NO: 90 - protein, 12 aa, Canis sp.
CQVGDSGSKAVF

SEQ ID NO: 91 - DNA, 361 bp, Canis sp.
  source 1..361: mol_type = other DNA
gaggtgcagctggtggagtctgggggagacctggtgaagcctggggggtccctgagactttcctgtgtggcctctggattcaccttcagtagttatcacatgagctgggtccgccaggctccagggaaggggcttcagtgggtcgcatacattaccagtggtggaagaaccacaagctatgcagacgctgtgaagggccgattcaccatctccagagacaatgccaagaacacgttgtatcttcagatgaaaagcctgagagccgaggacacggccgtgtattactgtgcgagtgatctggttctagtattagggggggctgagtactggggccagggaaccctggtcaccgtctcctcag

SEQ ID NO: 92 - protein, 120 aa, Canis sp.
EVQLVESGGDLVKPGGSLRLSCVASGFTFSSYHMSWVRQAPGKGLQWVAYITSGGRTTSYADAVKGRFTISRDNAKNTLYLQMKSLRAEDTAVYYCASDLVLVLGGAEYWGQGTLVTVSS

SEQ ID NO: 93 - DNA, 322 bp, Canis sp.
  source 1..322: mol_type = other DNA
tcctatgagctgactcagccaccatccgtgaatgtgaccctgagggagacggcccacatcacctgtgggggagacagcattggaaggaaatatgttcaatggatccagcagaatccaggccaggcccccgtcgtgattatctataaagatagcaacaggccgacagggatccctgagcgattctctggcgccaactcagggaacacggctaccctgaccatcagtggggccctggccgaagacgaggctgactattactgccaggtgggggacagtggttctaaggctgtgttcggcggaggcacccacctgaccgtcctcg

SEQ ID NO: 94 - protein, 107 aa, Canis sp.
SYELTQPPSVNVTLRETAHITCGGDSIGRKYVQWIQQNPGQAPVVIIYKDSNRPTGIPERFSGANSGNTATLTISGALAEDEADYYCQVGDSGSKAVFGGGTHLTVL

SEQ ID NO: 95 - protein, 7 aa, Canis sp.
GFTFSSY

SEQ ID NO: 96 - protein, 6 aa, Canis sp.
TSGGRT

SEQ ID NO: 97 - protein, 15 aa, Canis sp.
CASDLVLVLGGAEYW

SEQ ID NO: 98 - protein, 11 aa, Canis sp.
GGDSIGRKYVQ

SEQ ID NO: 99 - protein, 7 aa, Canis sp.
KDSNRPT

SEQ ID NO: 100 - protein, 12 aa, Canis sp.
CQVGDSGSKAVF

SEQ ID NO: 101 - DNA, 361 bp, Canis sp.
  source 1..361: mol_type = other DNA
gaggtgcagctggtggagtctgggggagacctggtgaagcctggggggtccctgagactttcctgtgtggcctctggattcaccttcagtaattatcacatgagctgggtccgccaggctccagggaaggggcttcagtgggtcgcatacattgtcagtggtggaagtagcacaacctatgcagacgctgtgaagggccgattcaccatctccagagacaacgccaagaacacgctgtatcttcagatgaccagcctacgagccgaggatacggccgtgtattactgtgcgagtgatctggttctactagtagggggggctgactactggggccagggaaccctggtcaccgtctcctcag

SEQ ID NO: 102 - protein, 120 aa, Canis sp.
EVQLVESGGDLVKPGGSLRLSCVASGFTFSNYHMSWVRQAPGKGLQWVAYIVSGGSSTTYADAVKGRFTISRDNAKNTLYLQMTSLRAEDTAVYYCASDLVLLVGGADYWGQGTLVTVSS

SEQ ID NO: 103 - DNA, 322 bp, Canis sp.
  source 1..322: mol_type = other DNA
tcctatgagctgactcagccaccatccgtgaatgtgaccctgagggagacggcccacatcacctgtgggggagacagcattggaagtaaatatgttcaatggatccagcagaatccaggccaggcccccgtggtgattatctataaagatagcaacaggccgacagggatccctgagcgattctctggcgccaactcagggaacacggctaccctgaccatcagtggggccctggccgaagacgaggctgactattactgccaggtgggggacagtggtactaaggctgtgttcggcggaggcacccacctgaccgtcctcg

SEQ ID NO: 104 - protein, 107 aa, Canis sp.
SYELTQPPSVNVTLRETAHITCGGDSIGSKYVQWIQQNPGQAPVVIIYKDSNRPTGIPERFSGANSGNTATLTISGALAEDEADYYCQVGDSGTKAVFGGGTHLTVL

SEQ ID NO: 105 - protein, 7 aa, Canis sp.
GFTFSNY

SEQ ID NO: 106 - protein, 6 aa, Canis sp.
VSGGSS

SEQ ID NO: 107 - protein, 15 aa, Canis sp.
CASDLVLLVGGADYW

SEQ ID NO: 108 - protein, 11 aa, Canis sp.
GGDSIGSKYVQ

SEQ ID NO: 109 - protein, 7 aa, Canis sp.
KDSNRPT

SEQ ID NO: 110 - protein, 12 aa, Canis sp.
CQVGDSGTKAVF

SEQ ID NO: 111 - DNA, 358 bp, Canis sp.
  source 1..358: mol_type = other DNA
gaggtgcagctggtggagtctgggggagacctggtgaagcctggggggtccctgagactctcctgtgtggcctctggattcaccttcagtaacttcgaaatgtactgggtccgccaggctccagggaaagggctggagtgggtcgcaaggatttataagagtggaagtaccacataccattcagaacgtgtaaagggccgattcaccatctccaaagacagcgccacgaacatggcgtttctgcagatgaacagcctgagagccgaggacacggccgtatattactgtacgagtgctagttctactggttggcgttttgactactggggccagggaaccctggtcaccgtctcctcag

SEQ ID NO: 112 - protein, 119 aa, Canis sp.
EVQLVESGGDLVKPGGSLRLSCVASGFTFSNFEMYWVRQAPGKGLEWVARIYKSGSTTYHSERVKGRFTISKDSATNMAFLQMNSLRAEDTAVYYCTSASSTGWRFDYWGQGTLVTVSS

SEQ ID NO: 113 - DNA, 319 bp, Canis sp.
  source 1..319: mol_type = other DNA
tcctatgtgctgacacagttgccatccgtgagtgtgaccctgaggcagacggcccgcatcacctgtgggggagacagcattggaagaaaaaatgtttgctggttccagcagaagctgggccaggcccctgtactgattatctatgatgataacagcaggccgtcagggatccctgagcgattctccggcgccaactcggggaacacggccaccctgaccatcagcggggccctggccgaggatgaggctgactattactgccaggtgtgggacagcagtactggtatattcggcggaggcacccatctgaccgtcctcg

SEQ ID NO: 114 - protein, 106 aa, Canis sp.
SYVLTQLPSVSVTLRQTARITCGGDSIGRKNVCWFQQKLGQAPVLIIYDDNSRPSGIPERFSGANSGNTATLTISGALAEDEADYYCQVWDSSTGIFGGGTHLTVL

SEQ ID NO: 115 - protein, 7 aa, Canis sp.
GFTFSNF

SEQ ID NO: 116 - protein, 6 aa, Canis sp.
YKSGST

SEQ ID NO: 117 - protein, 14 aa, Canis sp.
CTSASSTGWRFDYW

SEQ ID NO: 118 - protein, 11 aa, Canis sp.
GGDSIGRKNVC

SEQ ID NO: 119 - protein, 7 aa, Canis sp.
DDNSRPS

SEQ ID NO: 120 - protein, 11 aa, Canis sp.
CQVWDSSTGIF

SEQ ID NO: 121 - DNA, 361 bp, Canis sp.
  source 1..361: mol_type = other DNA
gaggtgcagttggtggagtctgggggagacctggtgaagcctggggggtccctgagactttcctgtgtggcctctggattcaccttcagtagttatcacatgagctgggtccgccaggctccagggaaggggcttcagtgggtcgcatacattaccagtggtggaagaaccacaagctatgcagacgctgtgaagggccgattcaccatctccagagacaacgccaagaacacgctgtatcttcagatgaacagcctgagagccgaggacacggccgtgtattactgtgcgagtgatctggttctagtattagggggggctgactactggggccagggaaccctggtcaccgtctcctcag

SEQ ID NO: 122 - protein, 120 aa, Canis sp.
EVQLVESGGDLVKPGGSLRLSCVASGFTFSSYHMSWVRQAPGKGLQWVAYITSGGRTTSYADAVKGRFTISRDNAKNTLYLQMNSLRAEDTAVYYCASDLVLVLGGADYWGQGTLVTVSS

SEQ ID NO: 123 - DNA, 322 bp, Canis sp.
  source 1..322: mol_type = other DNA
tcctatgagctgactcagccaccatccgtgaatgtgaccctgagggagacggcccacatcacctgtgggggagacagcattggaaggaaatatgttcaatggatccagcagaatccaggccaggcccccgtggtgattatctataaagataccaacaggccgacagggatccctgagcgattctctggcgccaactcagggaacacggctaccctgaccatcagtggggccctggccgaagacgaggctgactattactgccaggtgggggacagtggtactaaggctgtgttcggcggaggcacccacctgaccgtcctcg

SEQ ID NO: 124 - protein, 107 aa, Canis sp.
SYELTQPPSVNVTLRETAHITCGGDSIGRKYVQWIQQNPGQAPVVIIYKDTNRPTGIPERFSGANSGNTATLTISGALAEDEADYYCQVGDSGTKAVFGGGTHLTVL

SEQ ID NO: 125 - protein, 7 aa, Canis sp.
GFTFSSY

SEQ ID NO: 126 - protein, 6 aa, Canis sp.
TSGGRT

SEQ ID NO: 127 - protein, 15 aa, Canis sp.
CASDLVLVLGGADYW

SEQ ID NO: 128 - protein, 11 aa, Canis sp.
GGDSIGRKYVQ

SEQ ID NO: 129 - protein, 7 aa, Canis sp.
KDTNRPT

SEQ ID NO: 130 - protein, 12 aa, Canis sp.
CQVGDSGTKAVF

SEQ ID NO: 131 - DNA, 358 bp, Canis sp.
  source 1..358: mol_type = other DNA
gaggtgcagctgctggagtctgggggagacctggtgaagcctggggggtccctgagactctcctgtgtggcctctggattcaccttcagtaactacgaaatgtactgggtccgccaggctccagggaaagggctggagtgggtcgcaaggttttatgagagtggaagtaccacatactctgcagaagctgttccgggccgattcaccatctccagagacaacgccaagaacatggcctatctgcagatgaacagcctgagagccgaggacacggccgtatattactgtgcgagtgctagttctagttcttggcgttttgactactggggccagggaaccctggtcaccgtctcctcag

SEQ ID NO: 132 - protein, 119 aa, Canis sp.
EVQLLESGGDLVKPGGSLRLSCVASGFTFSNYEMYWVRQAPGKGLEWVARFYESGSTTYSAEAVPGRFTISRDNAKNMAYLQMNSLRAEDTAVYYCASASSSSWRFDYWGQGTLVTVSS

SEQ ID NO: 133 - DNA, 319 bp, Canis sp.
  source 1..319: mol_type = other DNA
tcctatgtgctgacacagctgccatccgtgagtgtgaccctggggcagacggcccgcatcacctgtgggggagacagcattggaagtaaaaatgtttactggttccagcagaagctgggccaggcccctgtactgattatctatgatgatcgcagcaggccgtcagggatccctgagcgattctccggcgccaactcggggaacacggccaccctgaccatcagcggggccctggccgaggatgaggctgactattactgccaggtgtgggacagcagtactggtgtgttcggcggaggcacccatctgaccgtcctcg

SEQ ID NO: 134 - protein, 106 aa, Canis sp.
SYVLTQLPSVSVTLGQTARITCGGDSIGSKNVYWFQQKLGQAPVLIIYDDRSRPSGIPERFSGANSGNTATLTISGALAEDEADYYCQVWDSSTGVFGGGTHLTVL

SEQ ID NO: 135 - protein, 7 aa, Canis sp.
GFTFSNY

SEQ ID NO: 136 - protein, 6 aa, Canis sp.
YESGST

SEQ ID NO: 137 - protein, 14 aa, Canis sp.
CASASSSSWRFDYW

SEQ ID NO: 138 - protein, 11 aa, Canis sp.
GGDSIGSKNVY

SEQ ID NO: 139 - protein, 7 aa, Canis sp.
DDRSRPS

SEQ ID NO: 140 - protein, 11 aa, Canis sp. [sequence omitted]

SEQ ID NO: 141 - DNA, 361 bp, Canis sp.
  source 1..361: mol_type = other DNA
gaggtgcagctggtggagtctgggggagacctggtgaagcctggggggtccctgagactttcctgtgtggcctctggattcaccttcagtagttatcacatgagctgggtccgccaggctccagggaaggggcttcagtgggtcgcatacattaccagtggtggaagaaccacaagctatgcagacgctgtgaagggccgattcaccatctccagagacaatgccaagaacacgttgtatcttcagatgaaaagcctgagagccgaggacacggccgtgtattactgtgcgagggatctggttctagtattagggggggctgagtactggggccagggaaccctggtcaccgtctcctcag

SEQ ID NO: 142 - protein, 120 aa, Canis sp.
EVQLVESGGDLVKPGGSLRLSCVASGFTFSSYHMSWVRQAPGKGLQWVAYITSGGRTTSYADAVKGRFTISRDNAKNTLYLQMKSLRAEDTAVYYCARDLVLVLGGAEYWGQGTLVTVSS

SEQ ID NO: 143 - DNA, 322 bp, Canis sp.; identical to SEQ ID NO: 93
  source 1..322: mol_type = other DNA

SEQ ID NO: 144 - protein, 107 aa, Canis sp.; identical to SEQ ID NO: 94

SEQ ID NO: 145 - protein, 7 aa, Canis sp.
GFTFSSY

SEQ ID NO: 146 - protein, 6 aa, Canis sp.
TSGGRT

SEQ ID NO: 147 - protein, 15 aa, Canis sp.
CARDLVLVLGGAEYW

SEQ ID NO: 148 - protein, 11 aa, Canis sp.
GGDSIGRKYVQ

SEQ ID NO: 149 - protein, 7 aa, Canis sp.
KDSNRPT

SEQ ID NO: 150 - protein, 12 aa, Canis sp.
CQVGDSGSKAVF

SEQ ID NO: 151 - DNA, 370 bp, Canis sp.
  source 1..370: mol_type = other DNA
gaggtgcagctggtggagtctgggggagaccttgtgaaacctgaggggtccctgagactctcctgtgtggtctctggcttcaccttcattaccaacgacatgagctgggtccgccaggctccagggaaggggctgcagtgggtcgcatacattaccaatgatggaaggtacacaagttacacagacgctgtgaagggccgattcaccatctccagagacaatgccaagaacacgctgtatctgcagatgaacagcctgagaactgaggacacagccgtttattactgtgcgaagcatagtagcaactactacagggggaggtttggtatggactactggggccatggcacctcactcttcgtgtcctcag

SEQ ID NO: 152 - protein, 123 aa, Canis sp.
EVQLVESGGDLVKPEGSLRLSCVVSGFTFITNDMSWVRQAPGKGLQWVAYITNDGRYTSYTDAVKGRFTISRDNAKNTLYLQMNSLRTEDTAVYYCAKHSSNYYRGRFGMDYWGHGTSLFVSS

SEQ ID NO: 153 - DNA, 319 bp, Canis sp.
  source 1..319: mol_type = other DNA
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcaccaggccgacagggatccctgagcgattctccggcgccaactcggggaacacggccaccctgaccatcagcggggccctggccgaggacgaggctgactattactgccaggtgtgggacagcagtgctagtgtgttcggcggaggcacccatctgaccgtcctcg

SEQ ID NO: 154 - protein, 106 aa, Canis sp.
SYVLTQLPSKNVTLKQPAHITCGGDNIGSKSVHWYQQKLGQAPVLIIYYDSTRPTGIPERFSGANSGNTATLTISGALAEDEADYYCQVWDSSASVFGGGTHLTVL

SEQ ID NO: 155 - protein, 7 aa, Canis sp.
GFTFITN

SEQ ID NO: 156 - protein, 6 aa, Canis sp.
TNDGRY

SEQ ID NO: 157 - protein, 18 aa, Canis sp.
CAKHSSNYYRGRFGMDYW

SEQ ID NO: 158 - protein, 11 aa, Canis sp.
GGDNIGSKSVH

SEQ ID NO: 159 - protein, 7 aa, Canis sp.
YDSTRPT

SEQ ID NO: 160 - protein, 11 aa, Canis sp.
CQVWDSSASVF

SEQ ID NO: 161 - DNA, 358 bp, Canis sp.
  source 1..358: mol_type = other DNA
gaggtgcagctggtggagtctgggggagacctggtgaagcctggggggtccctgagactctcctgtgtggcctctggattcaccttcagtaacttcgaaatgtactgggtccgccaggctccagggaaagggctggagtgggtcgcaaggatttatgagagtggaactaccacataccatgcagaaggtgtaaagggccgattcaccatttccaaagacaacgtcaagaacatggcgtatctgcagatgaacagcctgagagccgaggacacggccgtatattactgtacgagtgctagttctagtagttggcgttttgactactggggccagggaaccctggtcaccgtctcctcag

SEQ ID NO: 162 - protein, 119 aa, Canis sp.
EVQLVESGGDLVKPGGSLRLSCVASGFTFSNFEMYWVRQAPGKGLEWVARIYESGTTTYHAEGVKGRFTISKDNVKNMAYLQMNSLRAEDTAVYYCTSASSSSWRFDYWGQGTLVTVSS

SEQ ID NO: 163 - DNA, 319 bp, Canis sp.
  source 1..319: mol_type = other DNA
tcctatgtgctgacacagctgccatccgtgagtgtgaccctgaggcagacggcccgcatcacctgtgggggagacagcattggaagaaaaaatgtttgctggttccagcagaagctgggccaggcccctatactgattatctatgatgatagcagcaggccgtcagggatccctgagcgattctccggcgccaactcggggaacacggccaccctgaccatcagcggggccctggccgaggatgaggctgactattactgccaggtgtgggacagcagtactggtatattcggcggaggcacccatctgaccgtcctcg

SEQ ID NO: 164 - protein, 106 aa, Canis sp.
SYVLTQLPSVSVTLRQTARITCGGDSIGRKNVCWFQQKLGQAPILIIYDDSSRPSGIPERFSGANSGNTATLTISGALAEDEADYYCQVWDSSTGIFGGGTHLTVL

SEQ ID NO: 165 - protein, 7 aa, Canis sp.
GFTFSNF

SEQ ID NO: 166 - protein, 6 aa, Canis sp.
YESGTT

SEQ ID NO: 167 - protein, 14 aa, Canis sp.
CTSASSSSWRFDYW

SEQ ID NO: 168 - protein, 11 aa, Canis sp.
GGDSIGRKNVC

SEQ ID NO: 169 - protein, 7 aa, Canis sp.
DDSSRPS

SEQ ID NO: 170 - protein, 11 aa, Canis sp.
CQVWDSSTGIF

SEQ ID NO: 171 - DNA, 370 bp, Canis sp.
  source 1..370: mol_type = other DNA
gaggtgcatctggtggagtctgggggagaccttgtgaaacctgaggggtccctgagactctcctgtgtggtctctggcttcaccttcactactaacgacatgagctgggtccgccaggctccagggaaggggctgcagtgggtcgcatacattaccagtgatggaaggatcacaacttacacagacgctgtgaagggccgattcaccatctccagagacaatgccaagaacacgctgtatctgcagatgaacagcctgagaactgaggacacagccgtgtattattgtgcgaaacatagtagtaactggtccagggggaggtttggtttggactactggggccatggcacctcactcttcgtgtcctcag

SEQ ID NO: 172 - protein, 123 aa, Canis sp.
EVHLVESGGDLVKPEGSLRLSCVVSGFTFTTNDMSWVRQAPGKGLQWVAYITSDGRITTYTDAVKGRFTISRDNAKNTLYLQMNSLRTEDTAVYYCAKHSSNWSRGRFGLDYWGHGTSLFVSS

SEQ ID NO: 173 - DNA, 319 bp, Canis sp.
  source 1..319: mol_type = other DNA
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacaacagtgctagtgtgttcggcggaggcacccatctgaccgtcctcg

SEQ ID NO: 174 - protein, 106 aa, Canis sp.
SYVLTQLPSKNVTLKQPAHITCGGDNIGSKSVHWYQQKLGQAPVLIIYYDSSRPTGIPERFSGANSGNTATLTISGALAEDEADYYCQVWDNSASVFGGGTHLTVL

SEQ ID NO: 175 - protein, 7 aa, Canis sp.
GFTFTTN

SEQ ID NO: 176 - protein, 6 aa, Canis sp.
TSDGRI

SEQ ID NO: 177 - protein, 18 aa, Canis sp.
CAKHSSNWSRGRFGLDYW

SEQ ID NO: 178 - protein, 11 aa, Canis sp.
GGDNIGSKSVH

SEQ ID NO: 179 - protein, 7 aa, Canis sp.
YDSSRPT

SEQ ID NO: 180 - protein, 11 aa, Canis sp.
CQVWDNSASVF

SEQ ID NO: 181 - DNA, 361 bp, Canis sp.
  source 1..361: mol_type = other DNA
gaagtgcagctggtggagtctgggggagacctcgtgaagcctggggggtccctgagactttcctgtgtggcctctggattcaccttcagtagttatcacatgagctgggtccgccaggctccagggaaggggcttcagtgggtcgcatacattaccagtggtggaagtaccacaagctatgcagacgctgtgaagggccgattcaccatctccagagacaacgccaagaacacgctgtatcttcagatggacagcctgagagccgacgacacggccgtgtattactgtgcgagtgatctggttctgctattagggggggctgactactggggccagggaaccctggtcaccgtctcctcag

SEQ ID NO: 182 - protein, 120 aa, Canis sp.
EVQLVESGGDLVKPGGSLRLSCVASGFTFSSYHMSWVRQAPGKGLQWVAYITSGGSTTSYADAVKGRFTISRDNAKNTLYLQMDSLRADDTAVYYCASDLVLLLGGADYWGQGTLVTVSS

SEQ ID NO: 183 - DNA, 322 bp, Canis sp.
  source 1..322: mol_type = other DNA
tcctatgagctgactcagccaccatccgtgaatgtgaccctgagggagacggcccacatcacctgtgggggagacagcattggaaggaaatatgttcaatggatccagcagcatccaggccaggcccccgtggtgattatctataaagattacaacaggccgacagggatccctgagcgattctctggcgccaactcagggaacacggctaccctgaccatcagtggggccctggccgaagacgaggctgactattactgccaggtgggggacagtggtactaaggctgtgttcggcggaggcacccacctgaccgtcctcg

SEQ ID NO: 184 - protein, 107 aa, Canis sp.
SYELTQPPSVNVTLRETAHITCGGDSIGRKYVQWIQQHPGQAPVVIIYKDYNRPTGIPERFSGANSGNTATLTISGALAEDEADYYCQVGDSGTKAVFGGGTHLTVL

SEQ ID NO: 185 - protein, 7 aa, Canis sp.
GFTFSSY

SEQ ID NO: 186 - protein, 6 aa, Canis sp.
TSGGST

SEQ ID NO: 187 - protein, 15 aa, Canis sp.
CASDLVLLLGGADYW

SEQ ID NO: 188 - protein, 11 aa, Canis sp.
GGDSIGRKYVQ

SEQ ID NO: 189 - protein, 7 aa, Canis sp.
KDYNRPT

SEQ ID NO: 190 - protein, 12 aa, Canis sp.
CQVGDSGTKAVF

SEQ ID NO: 191 - DNA, 370 bp, Canis sp.
  source 1..370: mol_type = other DNA
gaggtgcagctggtggagtctgggggagaccttgtgaaacctgaggggtccctgagactctcctgtgtggtctctggcttcaccttcagtagctacgacatgagctgggtccgccaggctccagggaaggggctgcagtgggtcgcatacatcaccaatgacggaaggtacacaagttacacagacgctgtgaagggccgattcaccatctccagagacaatgccaagaacacgctgtatctgcagatgaacagcctgagaactgaggacacagccatctattactgtgcgaagcatggtagcagctggtccagggggagatttgctatggactactggggccaaggcacctcactcttcgtgtcctcag

SEQ ID NO: 192 - protein, 123 aa, Canis sp.
EVQLVESGGDLVKPEGSLRLSCVVSGFTFSSYDMSWVRQAPGKGLQWVAYITNDGRYTSYTDAVKGRFTISRDNAKNTLYLQMNSLRTEDTAIYYCAKHGSSWSRGRFAMDYWGQGTSLFVSS

SEQ ID NO: 193 - DNA, 319 bp, Canis sp.
  source 1..319: mol_type = other DNA
tcctatgtgctgacacagctgccatccaaaaatgtgaccctgaaccagccggcccacatcacctgtgggggagacagcattggaactaaaagtgttcactggtaccagcagaagctgggccaggcccctgtcctgattatcttttatgatagcagcaggccgacagggatccctgagcgattctccggcgccaactcggggaacacggccaccctgaccatcagcggggccctggccgaggacgaggctgactattactgccaggtgtgggacagcaatgctagtgtgttcggcggaggcacccatctgaccgtcctcg

SEQ ID NO: 194 - protein, 106 aa, Canis sp.
SYVLTQLPSKNVTLNQPAHITCGGDSIGTKSVHWYQQKLGQAPVLIIFYDSSRPTGIPERFSGANSGNTATLTISGALAEDEADYYCQVWDSNASVFGGGTHLTVL

SEQ ID NO: 195 - protein, 7 aa, Canis sp.
GFTFSSY

SEQ ID NO: 196 - protein, 6 aa, Canis sp.
TNDGRY

SEQ ID NO: 197 - protein, 18 aa, Canis sp.
CAKHGSSWSRGRFAMDYW

SEQ ID NO: 198 - protein, 11 aa, Canis sp.
GGDSIGTKSVH

SEQ ID NO: 199 - protein, 7 aa, Canis sp.
YDSSRPT

SEQ ID NO: 200 - protein, 11 aa, Canis sp.
CQVWDSNASVF

SEQ ID NO: 201 - DNA, 370 bp, Canis sp.
  source 1..370: mol_type = other DNA
gaggtgcagctggtggagtctgggggagaccctgtgaaacctgaggggtcccagagactctcctgtgtggtctctggcttcaccttcagtagctacgacatgagctgggtccgccaggctccagggaaggggctgcagtgggtcgcatacattaccagtgatggaaggagcacaagttacacagacgctgtgaagggccgattcaccatctccagagacaatgccaggaacacgctgtttctgcagatgaacagcctgagaactgaggacacagccgtgtattactgtgcgaggcatagtagcagctggtccagggggaggtttggtatggactactggggccatggcacctcgctcttcgtgtcctcag

SEQ ID NO: 202 - protein, 123 aa, Canis sp.
EVQLVESGGDPVKPEGSQRLSCVVSGFTFSSYDMSWVRQAPGKGLQWVAYITSDGRSTSYTDAVKGRFTISRDNARNTLFLQMNSLRTEDTAVYYCARHSSSWSRGRFGMDYWGHGTSLFVSS

SEQ ID NO: 203 - DNA, 319 bp, Canis sp.
  source 1..319: mol_type = other DNA
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctggtgtgttcggcggaggcacccatctgaccgtcctcg

SEQ ID NO: 204 - protein, 106 aa, Canis sp.
SYVLTQLPSKNVTLKQPAHITCGGDNIGSKSVHWYQQKLGQAPVLIIYYDSSRPTGIPERFSGANSGNTATLTISGALAEDEADYYCQVWDSSAGVFGGGTHLTVL

SEQ ID NO: 205 - protein, 7 aa, Canis sp.
GFTFSSY

SEQ ID NO: 206 - protein, 6 aa, Canis sp.
TSDGRS

SEQ ID NO: 207 - protein, 18 aa, Canis sp.
CARHSSSWSRGRFGMDYW

SEQ ID NO: 208 - protein, 11 aa, Canis sp.
GGDNIGSKSVH

SEQ ID NO: 209 - protein, 7 aa, Canis sp.
YDSSRPT

SEQ ID NO: 210 - protein, 11 aa, Canis sp. [sequence omitted]

SEQ ID NO: 211 - DNA, 361 bp, Canis sp.
  source 1..361: mol_type = other DNA
gaggtgcagctggtggagtctgggggagacctggtgaagcctggggggtccctgagactttcctgtgtggcctctggattcaccttcagtagttatcacatgacctgggtccgccaggctccagggaaggggcttcagtgggtcgcatacattaccagtggtggaattaccacaagctatgcagacgctgtgaagggccgattcaccatctccagagacaacgccaagaacacgctgtatcttcagatgagcagcctgagagccgaggacacggccatgtattactgtgcgagggatctggttctactattagggggggctgactactggggccagggaaccctggtcaccgtctcctcag

SEQ ID NO: 212 - protein, 120 aa, Canis sp.
EVQLVESGGDLVKPGGSLRLSCVASGFTFSSYHMTWVRQAPGKGLQWVAYITSGGITTSYADAVKGRFTISRDNAKNTLYLQMSSLRAEDTAMYYCARDLVLLLGGADYWGQGTLVTVSS

SEQ ID NO: 213 - DNA, 322 bp, Canis sp.
  source 1..322: mol_type = other DNA
tcctatgagctgactcagccaccatccgtgaatgtgaccctgagggagacggcccacatcacctgtgggggagacagcattggaaggaaatatgttcaatggatccagcagaatccaggccaggcccccgtggtgattatctataaagatagcaacaggccgacagggatccctgagcgattctctggcgccaactcagggaacacggctaccctgaccatcagtggggccctggccgaagacgaggctgactattactgccaggtgggggacagtggtactaaggctgtgttcggcggaggcacccacctgaccgtcctcg

SEQ ID NO: 214 - protein, 107 aa, Canis sp.
SYELTQPPSVNVTLRETAHITCGGDSIGRKYVQWIQQNPGQAPVVIIYKDSNRPTGIPERFSGANSGNTATLTISGALAEDEADYYCQVGDSGTKAVFGGGTHLTVL

SEQ ID NO: 215 - protein, 7 aa, Canis sp.
GFTFSSY

SEQ ID NO: 216 - protein, 6 aa, Canis sp.
TSGGIT

SEQ ID NO: 217 - protein, 15 aa, Canis sp.
CARDLVLLLGGADYW

SEQ ID NO: 218 - protein, 11 aa, Canis sp.
GGDSIGRKYVQ

SEQ ID NO: 219 - protein, 7 aa, Canis sp.
KDSNRPT

SEQ ID NO: 220 - protein, 12 aa, Canis sp.
CQVGDSGTKAVF

SEQ ID NO: 221 - DNA, 370 bp, Canis sp.
  source 1..370: mol_type = other DNA
gaggtgcagctggtggagtctgggggagaccttgtgaaacctgaggggtccctgagactctcctgtgtggtcgctggcttcaccttcaatagctacgacatgagctgggtccgccaggctccagggaaggggctgcagtgggtcgcatacattaccagtgatggaaggttcacaagttacacagacgctgtgaagggccgattcaccatctccagagacaatgccaagaacacgctctatctgcagatgagcagcctgagaactgaggacacagccgtgtattactgtgcgagacatagtagcagctggtacagggggaggtttggtatggacttctggggccatggcacctcactcttcgtgtcctcag

SEQ ID NO: 222 - protein, 123 aa, Canis sp.
EVQLVESGGDLVKPEGSLRLSCVVAGFTFNSYDMSWVRQAPGKGLQWVAYITSDGRFTSYTDAVKGRFTISRDNAKNTLYLQMSSLRTEDTAVYYCARHSSSWYRGRFGMDFWGHGTSLFVSS

SEQ ID NO: 223 - DNA, 319 bp, Canis sp.
  source 1..319: mol_type = other DNA
tcctatgtgctgacacagctgccatccaaaaatgtgaccctgaagcagccggcccacatcacctgtgggggagacaacattggaagtaaaagtgttcactggtaccagcagaagctgggccaggcccctatactgattatctattatgatagcagcaggccgacagggatccctgagcgattctccggcgccaactcggggaacacggccaccctgaccatcagcggggccctggccgaggacgaggctgactattactgccaggtgtgggacagcagtgctagtgtgttcggcggaggcacccatctgaccgtcctcg

SEQ ID NO: 224 - protein, 106 aa, Canis sp.
SYVLTQLPSKNVTLKQPAHITCGGDNIGSKSVHWYQQKLGQAPILIIYYDSSRPTGIPERFSGANSGNTATLTISGALAEDEADYYCQVWDSSASVFGGGTHLTVL

SEQ ID NO: 225 - protein, 7 aa, Canis sp.
GFTFNSY

SEQ ID NO: 226 - protein, 6 aa, Canis sp.
TSDGRF

SEQ ID NO: 227 - protein, 18 aa, Canis sp.
CARHSSSWYRGRFGMDFW

SEQ ID NO: 228 - protein, 11 aa, Canis sp.
GGDNIGSKSVH

SEQ ID NO: 229 - protein, 7 aa, Canis sp.
YDSSRPT

SEQ ID NO: 230 - protein, 11 aa, Canis sp.
CQVWDSSASVF

SEQ ID NO: 231 - DNA, 346 bp, Canis sp.
  source 1..346: mol_type = other DNA
gaggtgcagctggtggagtctgggggagacctggtgaagcctggggggtccctgagactgtcctgtgtggcctctggattcatcttcagtagctacagcatgagctgggtccgccaggctcctgagaaggggctgcagttggtcgcaggtattaacagcgctggaagtagcacgtactacacagacgctgtgaagggccgattcaccatctccagagacaacgccaagaacacagtgtatctgcagatgaacagcctgagagccgaggacacggccatttattactgtgcaaagcgttgggtttttgacttctggggccagggaaccctggtcaccgtctcctcag

SEQ ID NO: 232 - protein, 115 aa, Canis sp.
EVQLVESGGDLVKPGGSLRLSCVASGFIFSSYSMSWVRQAPEKGLQLVAGINSAGSSTYYTDAVKGRFTISRDNAKNTVYLQMNSLRAEDTAIYYCAKRWVFDFWGQGTLVTVSS

SEQ ID NO: 233 - DNA, 337 bp, Canis sp.
  source 1..337: mol_type = other DNA
catattgtcatgacacagaccccactgtccctgtccgtcagccctggagagccggcctccatctcctgcaaggccagtcagagcctcctgctcagttatggaaacacctatttgtattggttccgacagaagccaggccagtctccacagcgtttgatctataaggtctccaacagagaccctggggtcccagacaggttcagtggcagcgggtcagggacagatttcaccctgagaatcagcagagtggaggctgatgatgctggagtttattactgcgggcaaggtatacaagatccgttcacttttggccaagggaccaaactggagatcaaac

SEQ ID NO: 234 - protein, 112 aa, Canis sp.
HIVMTQTPLSLSVSPGEPASISCKASQSLLLSYGNTYLYWFRQKPGQSPQRLIYKVSNRDPGVPDRFSGSGSGTDFTLRISRVEADDAGVYYCGQGIQDPFTFGQGTKLEIK

SEQ ID NO: 235 - protein, 7 aa, Canis sp.
GFIFSSY

SEQ ID NO: 236 - protein, 6 aa, Canis sp.
NSAGSS

SEQ ID NO: 237 - protein, 10 aa, Canis sp.
CAKRWVFDFW

SEQ ID NO: 238 - protein, 16 aa, Canis sp.
KASQSLLLSYGNTYLY

SEQ ID NO: 239 - protein, 7 aa, Canis sp.
KVSNRDP

SEQ ID NO: 240 - protein, 11 aa, Canis sp.
CGQGIQDPFTF

SEQ ID NO: 241 - DNA, 358 bp, Canis sp.
  source 1..358: mol_type = other DNA
gaggtgcagctggtggagtctgggggagacctggtgaagcctggggggtccctgagactctcctgtgtggcctctggattcaccttcaataactacgaaatgtactgggtccgccaggctccagggaaagggctggagtgggtcgcaaggatttatgagagtggaacaaccacatattctgcagaagctgatacgggccgattcaccatctccagagacaacgccaagaacatggtttttctgcagatgaacagcctgagagccgaggacacggccgtctattactgtgcgagtgctagttctagtacttggcgttttgactactggggccagggaaccctggtcaccgtctcctcag

SEQ ID NO: 242 - protein, 119 aa, Canis sp.
EVQLVESGGDLVKPGGSLRLSCVASGFTFNNYEMYWVRQAPGKGLEWVARIYESGTTTYSAEADTGRFTISRDNAKNMVFLQMNSLRAEDTAVYYCASASSSTWRFDYWGQGTLVTVSS

SEQ ID NO: 243 - DNA, 319 bp, Canis sp.
  source 1..319: mol_type = other DNA
tcctatgtgctgacacaattgccttccgtgagtgtgaccctggggcagacggcccgcatcacctgtgggggagacagcattggaagaaaaaatgtttactggttccagcagaagctgggccaggcccctgtactgattatctatgatgatcgcagcaggccgtcagggatccctgagcgattctccggcgccaactcggggaacacggccaccctgaccatcagcggggccctggccgaggatgaggctgactattactgccaggtgtgggacagcagtactggtgtgttcggcggaggcacccatctgaccgtcctcg

SEQ ID NO: 244 - protein, 106 aa, Canis sp.
SYVLTQLPSVSVTLGQTARITCGGDSIGRKNVYWFQQKLGQAPVLIIYDDRSRPSGIPERFSGANSGNTATLTISGALAEDEADYYCQVWDSSTGVFGGGTHLTVL

SEQ ID NO: 245 - protein, 7 aa, Canis sp.
GFTFNNY

SEQ ID NO: 246 - protein, 6 aa, Canis sp.
YESGTT

SEQ ID NO: 247 - protein, 14 aa, Canis sp.
CASASSSTWRFDYW

SEQ ID NO: 248 - protein, 11 aa, Canis sp.
GGDSIGRKNVY

SEQ ID NO: 249 - protein, 7 aa, Canis sp.
DDRSRPS

SEQ ID NO: 250 - protein, 11 aa, Canis sp. [sequence omitted]

SEQ ID NO: 251 - DNA, 358 bp, Canis sp.
  source 1..358: mol_type = other DNA
gaggtgcagctggtggagtctgggggagacctggtgaagcctggggggtccctgagactctcctgtgtggcctctggattcaccttcaataactacgaaatgtactgggtccgccaggctccagggaaagggctggagtgggtcgcaaggatttatgagagtggaagtaccactttttctgcagaagctgttacgggccgattcaccatctccagagacaacgccaagaacatggtgtatctgcagatgaacagcctgagagccgaggacacggccgtgtattactgtgcgagtgctagttctagttcttggcgttttgactactggggccagggaaccctggtcaccgtctcctcag

SEQ ID NO: 252 - protein, 119 aa, Canis sp.
EVQLVESGGDLVKPGGSLRLSCVASGFTFNNYEMYWVRQAPGKGLEWVARIYESGSTTFSAEAVTGRFTISRDNAKNMVYLQMNSLRAEDTAVYYCASASSSSWRFDYWGQGTLVTVSS

SEQ ID NO: 253 - DNA, 319 bp, Canis sp.
  source 1..319: mol_type = other DNA
tcctatgtgctgacacagctgccatccgtgagtgtgaccctggggcagacggcccgcatcacctgtgggggagacagcattgggcgaaaaaatgtttgctggttccagcagaggctgggccaggcccctgaactgattatctatgatgatcgcagcaggccgtcagggatccctgagcgattctccggcgccaactcggggaacacggccaccctgaccatcagcggggccctggccgaggatgaggctgactattactgccaggtgtgggacagcagtactggtgtgttcggcggaggcacccatctgaccgtcctcg

SEQ ID NO: 254 - protein, 106 aa, Canis sp.
SYVLTQLPSVSVTLGQTARITCGGDSIGRKNVCWFQQRLGQAPELIIYDDRSRPSGIPERFSGANSGNTATLTISGALAEDEADYYCQVWDSSTGVFGGGTHLTVL

SEQ ID NO: 255 - protein, 7 aa, Canis sp.
GFTFNNY

SEQ ID NO: 256 - protein, 6 aa, Canis sp.
YESGST

SEQ ID NO: 257 - protein, 14 aa, Canis sp.
CASASSSSWRFDYW

SEQ ID NO: 258 - protein, 11 aa, Canis sp.
GGDSIGRKNVC

SEQ ID NO: 259 - protein, 7 aa, Canis sp.
DDRSRPS

SEQ ID NO: 260 - protein, 11 aa, Canis sp. [sequence omitted]

SEQ ID NO: 261 - DNA, 370 bp, Canis sp.
  source 1..370: mol_type = other DNA
gaggtgcagctggtggagtctgggggagaccttgtgaaacctgaggggtccctgagactctcctgtgtggtctctggcttcaccttcactacttacgacatgagctgggtccgccaggctccagggaaggggctgcagtgggtcgcatacattaccaatgatggaaggatcacaacttacacagacgctgtgaagggccgattcaccatctccagagacaatgccaagaacacgctgtatctgcagatgaacagcctgagaactgttgacacaggcgtgtattattgtgcgaggcatagtagcagctggtccagggggaggtttggtttggactactggggccatggtacttcagtcttcgtgtcctcag

SEQ ID NO: 262 - protein, 123 aa, Canis sp.
EVQLVESGGDLVKPEGSLRLSCVVSGFTFTTYDMSWVRQAPGKGLQWVAYITNDGRITTYTDAVKGRFTISRDNAKNTLYLQMNSLRTVDTGVYYCARHSSSWSRGRFGLDYWGHGTSVFVSS

SEQ ID NO: 263 - DNA, 319 bp, Canis sp.
  source 1..319: mol_type = other DNA
tcctatgtgctgacacagttgccatccaaaaatgtgaccctgaagcagccggcccacatcacctgtgggggagacaacattggaaggaaaggtgttcactggtaccagcagaagctgggccaggcccctgtattgattatctattatgatagcagcaggccgacagggatccctgagcgattctccggcgccaactcggggaacacggccaccctgaccatcagcggggccctggccgaggacgaggctgactattactgccaggtgtgggacaacagtgctagtgtgttcggcggaggcacccatctgaccgtcctcg

SEQ ID NO: 264 - protein, 106 aa, Canis sp.
SYVLTQLPSKNVTLKQPAHITCGGDNIGRKGVHWYQQKLGQAPVLIIYYDSSRPTGIPERFSGANSGNTATLTISGALAEDEADYYCQVWDNSASVFGGGTHLTVL

SEQ ID NO: 265 - protein, 7 aa, Canis sp.
GFTFTTY

SEQ ID NO: 266 - protein, 6 aa, Canis sp.
TNDGRI

SEQ ID NO: 267 - protein, 18 aa, Canis sp.
CARHSSSWSRGRFGLDYW

SEQ ID NO: 268 - protein, 11 aa, Canis sp.
GGDNIGRKGVH

SEQ ID NO: 269 - protein, 7 aa, Canis sp.
YDSSRPT

SEQ ID NO: 270 - protein, 11 aa, Canis sp.
CQVWDNSASVF

SEQ ID NO: 271 - DNA, 355 bp, Canis sp.
  source 1..355: mol_type = other DNA
gaggtacagttggtggagtctgggggagacctggtgaagccgggggggtccctgagactttcctgtgtggcctctggattctccttcaatacctaccacatgaactgggtccgccaggctccagggaaggggcttcactgggtcgcattcattaacagcgatgggggtggcacaagttatgcagacactgtgaagggccgattcaccatctccagagactacgccaagaatacactctatcttcagatgaatagtctgagagccgaagacacggccgtttattattgtgtgagtggatctatatggactcattttgactactggggccagggaaccctggtcaccgtctcctcag

SEQ ID NO: 272 - protein, 118 aa, Canis sp.
EVQLVESGGDLVKPGGSLRLSCVASGFSFNTYHMNWVRQAPGKGLHWVAFINSDGGGTSYADTVKGRFTISRDYAKNTLYLQMNSLRAEDTAVYYCVSGSIWTHFDYWGQGTLVTVSS

SEQ ID NO: 273 - DNA, 322 bp, Canis sp.
  source 1..322: mol_type = other DNA
tcctatgtgctgacacagctgccatccgtgagtatgaccctgaggcagacggcccacatcacctgtgggggagacatcattggaagtaaaagtgtttattggtaccagcagaagctgggccaggcccctgtactgattatttatagagataccaacaggccgacagggatccctgagcgattctctggcgccagctcggggaacacggccaccctgaccatcagcggggccctggccgaggacgaggctgactattactgccaggtttgggacagcagtactcaggctgtgttcggcggaggcacccatctgaccgtcctcg

SEQ ID NO: 274 - protein, 107 aa, Canis sp.
SYVLTQLPSVSMTLRQTAHITCGGDIIGSKSVYWYQQKLGQAPVLIIYRDTNRPTGIPERFSGASSGNTATLTISGALAEDEADYYCQVWDSSTQAVFGGGTHLTVL

SEQ ID NO: 275 - protein, 7 aa, Canis sp.
GFSFNTY

SEQ ID NO: 276 - protein, 6 aa, Canis sp.
NSDGGG

SEQ ID NO: 277 - protein, 13 aa, Canis sp.
CVSGSIWTHFDYW

SEQ ID NO: 278 - protein, 11 aa, Canis sp.
GGDIIGSKSVY

SEQ ID NO: 279 - protein, 7 aa, Canis sp.
RDTNRPT

SEQ ID NO: 280 - protein, 12 aa, Canis sp.
CQVWDSSTQAVF

SEQ ID NO: 281 - DNA, 364 bp, Canis sp.
  source 1..364: mol_type = other DNA
gaggtgcagctggtggagtctgggggagacctggtgaagcctgcggggtccctgagactgtcctgtgtggcctctggattcaccttcagtagctacagcatgagctgggtccgccaggctcctgagaaggggctgcagttggtcgcaggtattagcggcggtggaagtcgcacatactactcagacgctgtgaagggccgattcaccatctccagagacaacgccaagaacacagtgtatctgcagatgaacagcctgagagccgaggacacggccatttattactgtgcaaaggatggtggcagctgggcgggatataattttgactactggggccagggaaccctggtcaccgtctcctcag

SEQ ID NO: 282 - protein, 121 aa, Canis sp.
EVQLVESGGDLVKPAGSLRLSCVASGFTFSSYSMSWVRQAPEKGLQLVAGISGGGSRTYYSDAVKGRFTISRDNAKNTVYLQMNSLRAEDTAIYYCAKDGGSWAGYNFDYWGQGTLVTVSS

SEQ ID NO: 283 - DNA, 337 bp, Canis sp.
  source 1..337: mol_type = other DNA
gatattgtcatgacacagacgccaccgtccctgtctgtcagccctagagagacggcctccatctcctgcaaggccagtcagagcctcctgcacagtgatggaagcacctatttggattggtacctgcaaaagccaggccagtctccacagcttctgatatacttgggttccaaccgcttcactggcgtgtcagacaggttcagtggcagcgggtcagggacagaattcaccctgagaatcagcagagtggaggctaacgatactggagtttattactgcgggcaaggtacacagcttccttatactttcggccagggaaccgagctggagataaaac

SEQ ID NO: 284 - protein, 112 aa, Canis sp.
DIVMTQTPPSLSVSPRETASISCKASQSLLHSDGSTYLDWYLQKPGQSPQLLIYLGSNRFTGVSDRFSGSGSGTEFTLRISRVEANDTGVYYCGQGTQLPYTFGQGTELEIK

SEQ ID NO: 285 - protein, 7 aa, Canis sp.
GFTFSSY

SEQ ID NO: 286 - protein, 6 aa, Canis sp.
SGGGSR

SEQ ID NO: 287 - protein, 16 aa, Canis sp.
CAKDGGSWAGYNFDYW

SEQ ID NO: 288 - protein, 16 aa, Canis sp.
KASQSLLHSDGSTYLD

SEQ ID NO: 289 - protein, 7 aa, Canis sp.
LGSNRFT

SEQ ID NO: 290 - protein, 11 aa, Canis sp.
CGQGTQLPYTF

SEQ ID NO: 291 - DNA, 346 bp, Canis sp.
  source 1..346: mol_type = other DNA
gaggtgcagctggtggagtctgggggagacctggtgaagcctgcggggtccctaagactgtcctgtgtggcctctggtttcaccttcagtagctacagcatgagctgggtccgccaggctcctgagaaggggctgcagttggtcgcaggtattaacagcgctggaagtagcacatactacacagacgctgtgaagggccgattcaccatatccagagacaacgccaagaacacagtgtatttgcagatgaacagcctgagagccgaggacacggccatttattactgtgcaaagcgttgggtttttgactactggggccagggaaccctggtcaccgtctcctcag

SEQ ID NO: 292 - protein, 115 aa, Canis sp.
EVQLVESGGDLVKPAGSLRLSCVASGFTFSSYSMSWVRQAPEKGLQLVAGINSAGSSTYYTDAVKGRFTISRDNAKNTVYLQMNSLRAEDTAIYYCAKRWVFDYWGQGTLVTVSS

SEQ ID NO: 293 - DNA, 337 bp, Canis sp.
  source 1..337: mol_type = other DNA
gatattgtcatgacacagaccccactgtccctgtccgtcagccctggagagccggcctccatctcctgcaaggccagtcagagcctcccgcacagtcttgggaacacctatttatactggttccgacagaagccaggccagtctccacagcgtttgatctataaggtctccaacagagaccctggggtcccagacaggttcagtggcagcgggtcagggacagatttcaccctgagaatcagcagagtggaggctgatgatgctggaatttattactgcgggcaaggtatacaagatccgttcacttttggccaagggaccaaactggagatcaaac

SEQ ID NO: 294 - protein, 112 aa, Canis sp.
DIVMTQTPLSLSVSPGEPASISCKASQSLPHSLGNTYLYWFRQKPGQSPQRLIYKVSNRDPGVPDRFSGSGSGTDFTLRISRVEADDAGIYYCGQGIQDPFTFGQGTKLEIK

SEQ ID NO: 295 - protein, 7 aa, Canis sp.
GFTFSSY

SEQ ID NO: 296 - protein, 6 aa, Canis sp.
NSAGSS

SEQ ID NO: 297 - protein, 10 aa, Canis sp.
CAKRWVFDYW

SEQ ID NO: 298 - protein, 16 aa, Canis sp.
KASQSLPHSLGNTYLY

SEQ ID NO: 299 - protein, 7 aa, Canis sp.
KVSNRDP

SEQ ID NO: 300 - protein, 11 aa, Canis sp.
CGQGIQDPFTF

SEQ ID NO: 301 - DNA, 355 bp, Canis sp.
  source 1..355: mol_type = other DNA
gaggtgcacctggtggagtctgggggagaccttgtgaaacctgaggggtccctgagactctcctgtgtggtctctggcttcaccttcagtagctacgacatgagctgggtccgccaggctccagggaaggggctgcagtgggtcgcatacattagcagtgatggaaggagcacaggttacacagacgctgtgaagggccgattcaccatctccagagacaatgccaagaaaacgctgtatctgcagatgaacagcctgagaagtgaggacacagccgtttattactgtgcgaaggagacgcctaggtggggctttgactactggggccagggaaccctggtcaccgtctcctcag

SEQ ID NO: 302 - protein, 118 aa, Canis sp.
EVHLVESGGDLVKPEGSLRLSCVVSGFTFSSYDMSWVRQAPGKGLQWVAYISSDGRSTGYTDAVKGRFTISRDNAKKTLYLQMNSLRSEDTAVYYCAKETPRWGFDYWGQGTLVTVSS

SEQ ID NO: 303 - DNA, 319 bp, Canis sp.
  source 1..319: mol_type = other DNA
tcctatgtgctgacacagctgccatccgtgagtgtgaccctgaggcagacggcccgcatcacctgtgggggagccagtattggaagtaaaaatgtttactggcaccagcagaagctgggccaggcccctttactgattatctatgatgatagcagcaggccgtcagggatccctgagcgattctccggcgccaactcggggaacacggccaccctgaccatcagcggggccctggccgaagatgaggctgactattactgccaggtgtgggacagcagtactagtgtgttcggcggaggcacccatctgaccgtcctcg

SEQ ID NO: 304 - protein, 106 aa, Canis sp.
SYVLTQLPSVSVTLRQTARITCGGASIGSKNVYWHQQKLGQAPLLIIYDDSSRPSGIPERFSGANSGNTATLTISGALAEDEADYYCQVWDSSTSVFGGGTHLTVL

SEQ ID NO: 305 - protein, 7 aa, Canis sp.
GFTFSSY

SEQ ID NO: 306 - protein, 6 aa, Canis sp.
SSDGRS

SEQ ID NO: 307 - protein, 13 aa, Canis sp.
CAKETPRWGFDYW

SEQ ID NO: 308 - protein, 11 aa, Canis sp.
GGASIGSKNVY

SEQ ID NO: 309 - protein, 7 aa, Canis sp.
DDSSRPS

SEQ ID NO: 310 - protein, 11 aa, Canis sp.
CQVWDSSTSVF

SEQ ID NO: 311 - DNA, 355 bp, Canis sp.
  source 1..355: mol_type = other DNA
gaggtgcagctggaggagtctgggggagaccttgtgaaacctgaggggtccctgagactctcctgtgtggtctctggcttcaccctcagtaccttcggcatgagctgggtccgccaggctccagggaaggggctgcagtgggtcgcatacattagcagtgatggaaggagcacaagttacacagacgctgtgaagggccgattcaccatctccagagacaatgccaagaacacgctgtatctgcagatgaacagcctgcgaactgacgacacagccgtgtattactgtgtccagttgggccggggatttggtatagactactggggccatggcacctcactcttcgtgtcctcag

SEQ ID NO: 312 - protein, 118 aa, Canis sp.
EVQLEESGGDLVKPEGSLRLSCVVSGFTLSTFGMSWVRQAPGKGLQWVAYISSDGRSTSYTDAVKGRFTISRDNAKNTLYLQMNSLRTDDTAVYYCVQLGRGFGIDYWGHGTSLFVSS

SEQ ID NO: 313 - DNA, 319 bp, Canis sp.
  source 1..319: mol_type = other DNA
tcctatgtgctgacacagctgccatccaaaaatgtgaccctgaggcagccggcccacatcccctgtgggggagacaacattggaagtaaaagtgttcactggtaccagcagaagctgggccaggcccctgtactgattatctattatgataccagcaggccgacagggatccctgagcgattctccggcgccaactcggggaacacggccaccctgaccatcagcggggccctggccgaggacgaggctgactattactgccaggtgtgggacagcagtgctgctgtgttcggcggaggcacccacctgaccgtcctcg

SEQ ID NO: 314 - protein, 106 aa, Canis sp.
SYVLTQLPSKNVTLRQPAHIPCGGDNIGSKSVHWYQQKLGQAPVLIIYYDTSRPTGIPERFSGANSGNTATLTISGALAEDEADYYCQVWDSSAAVFGGGTHLTVL

SEQ ID NO: 315 - protein, 7 aa, Canis sp.
GFTLSTF

SEQ ID NO: 316 - protein, 6 aa, Canis sp.
SSDGRS

SEQ ID NO: 317 - protein, 13 aa, Canis sp.
CVQLGRGFGIDYW

SEQ ID NO: 318 - protein, 11 aa, Canis sp.
GGDNIGSKSVH

SEQ ID NO: 319 - protein, 7 aa, Canis sp.
YDTSRPT

SEQ ID NO: 320 - protein, 11 aa, Canis sp. [sequence omitted]

SEQ ID NO: 321 - DNA, 355 bp, Canis sp.
  source 1..355: mol_type = other DNA
gaggtgcagctggtggagtctgggggagaccttgtgaaacctgaggggtccctgagactctcctgtgtggtctctggcttcaccttgagtagctacggcatgagctgggtccgccaggctccagggagggggctgcagtgggtcgcatacattagcagtgatggaaggagcacaagttacacagacgctgtgaagggccgattcaccatctccagagacaatgccaagaacacgctgtatctgcagatgaacagcctgagaagtgaggacacagccgtgtattactgtgtccagttgggccggggctttggcatggactattggggccatggcacctcactcttcgtgtcctcag

SEQ ID NO: 322 - protein, 118 aa, Canis sp.
EVQLVESGGDLVKPEGSLRLSCVVSGFTLSSYGMSWVRQAPGRGLQWVAYISSDGRSTSYTDAVKGRFTISRDNAKNTLYLQMNSLRSEDTAVYYCVQLGRGFGMDYWGHGTSLFVSS

SEQ ID NO: 323 - DNA, 319 bp, Canis sp.
  source 1..319: mol_type = other DNA
tcctatgtgctgacacagctgccatccaaaaatgtgaccctgaagcagccggcccacatcacctgtgggggagacaacattggaagtaaaagcgttcactggtaccagcagaagctgggccaggcccctgtagtgattatctattatgatagtaccaggccgacagggatccctgagcgattctccggcgccaactcggggaacacggccaccctgaccatcagcggggccctggccgaggacgaggctgactattactgccaggtgtgggacagcagtgctgctgtgttcggcggaggcacccacctgaccgtcctcg

SEQ ID NO: 324 - protein, 106 aa, Canis sp.
SYVLTQLPSKNVTLKQPAHITCGGDNIGSKSVHWYQQKLGQAPVVIIYYDSTRPTGIPERFSGANSGNTATLTISGALAEDEADYYCQVWDSSAAVFGGGTHLTVL

SEQ ID NO: 325 - protein, 7 aa, Canis sp.
GFTLSSY

SEQ ID NO: 326 - protein, 6 aa, Canis sp.
SSDGRS

SEQ ID NO: 327 - protein, 13 aa, Canis sp.
CVQLGRGFGMDYW

SEQ ID NO: 328 - protein, 11 aa, Canis sp.
GGDNIGSKSVH

SEQ ID NO: 329 - protein, 7 aa, Canis sp.
YDSTRPT

SEQ ID NO: 330 - protein, 11 aa, Canis sp. [sequence omitted]

SEQ ID NO: 331 - DNA, 355 bp, Canis sp.
  source 1..355: mol_type = other DNA
gaggtgcaactggtggagtctgggggagaccttgtgaaacctgaggggtccctgagactctcctgtgtggtctctggcttcaccttcaataactacggcatgagttgggtccgccaggctccagggaaggggctgcagtgggtcgcatacattagtattgatggaaggagcacaagttacacagacgctgtgaagggccgattcaccatctccagagacaatgccaagaacacgctgtatctgcatataaacagcctgagaactgaggacacagccgtgtattactgtacgaagttggggagggggttcacttttggttactggggccagggcaccctggtcactgtctcctcag

SEQ ID NO: 332 - protein, 118 aa, Canis sp.
EVQLVESGGDLVKPEGSLRLSCVVSGFTFNNYGMSWVRQAPGKGLQWVAYISIDGRSTSYTDAVKGRFTISRDNAKNTLYLHINSLRTEDTAVYYCTKLGRGFTFGYWGQGTLVTVSS

SEQ ID NO: 333 - DNA, 319 bp, Canis sp.
  source 1..319: mol_type = other DNA
tcctatgtgctgacacagctgccatccaaaaatgtgaccctgaagcagccggcccacatcacctgtgggggagacaacattggaagtaaaagtgttcactggtaccagcagaagctgggccaggcccctgcactgattatctattatgatagcagcaggccgacagggatccctgagcgattctccggcgccaactcggggaacacggccaccctgaccatcagcggggccctggccgaggacgaggctgactattactgccaggtgtgggacagcagtgttagtgtgttcggcggaggcacccatctgaccgtcctcg

SEQ ID NO: 334 - protein, 106 aa, Canis sp.
SYVLTQLPSKNVTLKQPAHITCGGDNIGSKSVHWYQQKLGQAPALIIYYDSSRPTGIPERFSGANSGNTATLTISGALAEDEADYYCQVWDSSVSVFGGGTHLTVL

SEQ ID NO: 335 - protein, 7 aa, Canis sp.
GFTFNNY

SEQ ID NO: 336 - protein, 6 aa, Canis sp.
SIDGRS

SEQ ID NO: 337 - protein, 13 aa, Canis sp.
CTKLGRGFTFGYW

SEQ ID NO: 338 - protein, 11 aa, Canis sp.
GGDNIGSKSVH

SEQ ID NO: 339 - protein, 7 aa, Canis sp.
YDSSRPT

SEQ ID NO: 340 - protein, 11 aa, Canis sp.
CQVWDSSVSVF

SEQ ID NO: 341 - DNA, 355 bp, Canis sp.
  source 1..355: mol_type = other DNA
gaggtgcagctggtggagtctgggggagaccttgtgaaacctgaggggtccctgagactctcctgtgtggtctctgacttcaccttcggtagctacggcatgagctgggtccgccaggctccagggaaggggctgcagtgggtcgcatacattagcagtgatggaaggagcacaagttacacagacgctgtgaagggccgattcaccatctccagagacaatgccaagaacacgctacatctgcagatgaacagcctgagaactgaggacacagccgtgtattactgtgcgaagttggggagggggtttgcttttggttactggggccagggcaccctggtcactgtctcctcag

SEQ ID NO: 342 - protein, 118 aa, Canis sp.
EVQLVESGGDLVKPEGSLRLSCVVSDFTFGSYGMSWVRQAPGKGLQWVAYISSDGRSTSYTDAVKGRFTISRDNAKNTLHLQMNSLRTEDTAVYYCAKLGRGFAFGYWGQGTLVTVSS

SEQ ID NO: 343 - DNA, 319 bp, Canis sp.
  source 1..319: mol_type = other DNA
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcaacaggccgacagggatccctgagcgattctccggcgccaactcggggaacacggccaccctgaccatcagcggggccctggccgaggacgaggctgactattactgccaggtgtgggacagcagtgctagtgtgttcggcggaggcacccatctgaccgtcctcg

SEQ ID NO: 344 - protein, 106 aa, Canis sp.
SYVLTQLPSKNVTLKQPAHITCGGDNIGSKSVHWYQQKLGQAPVLIIYYDSNRPTGIPERFSGANSGNTATLTISGALAEDEADYYCQVWDSSASVFGGGTHLTVL

SEQ ID NO: 345 - protein, 7 aa, Canis sp.
DFTFGSY

SEQ ID NO: 346 - protein, 6 aa, Canis sp.
SSDGRS

SEQ ID NO: 347 - protein, 13 aa, Canis sp.
CAKLGRGFAFGYW

SEQ ID NO: 348 - protein, 11 aa, Canis sp.
GGDNIGSKSVH

SEQ ID NO: 349 - protein, 7 aa, Canis sp.
YDSNRPT

SEQ ID NO: 350 - protein, 11 aa, Canis sp.
CQVWDSSASVF

SEQ ID NO: 351 - DNA, 361 bp, Canis sp.
  source 1..361: mol_type = other DNA
gaggttcacctggtggagtctgggggagaccttgtgaaacctgaggggtccctgagactctcctgtgtggtctctggcttcaccttcagtaactacgtcatgaactgggtccgccaggctccagggaaggggctgcagtgggtcgcattcattagcggtgatggaaggagcacaacttacacagacgctgtgaagagccgattcaccatctccagagacaatgccaagaacacgctgtatctgcagatgagcagcctgagaactgaggacacagccatatacttctgtgcgaaggagaaatatagtagcggctggtcttttgagtactggggccagggaaccctggtcaccgtctcctcag

SEQ ID NO: 352 - protein, 120 aa, Canis sp.
EVHLVESGGDLVKPEGSLRLSCVVSGFTFSNYVMNWVRQAPGKGLQWVAFISGDGRSTTYTDAVKSRFTISRDNAKNTLYLQMSSLRTEDTAIYFCAKEKYSSGWSFEYWGQGTLVTVSS

SEQ ID NO: 353 - DNA, 319 bp, Canis sp.
  source 1..319: mol_type = other DNA
tcctatgtgctgacacagctgccatccaaaaatgtgaccctgaagcagccggcccacatcacctgtgggggagacaacattggaagtaaaagtgttcactggtaccagcagaagctgggccaggcccctgtattgattatctattatgacagcagcaggccgacagggatccctgagcgattctccggcgccaactcggggaacacggccaccctgaccatcagcggggccctggccgaggacgaggctgactattactgccaggtgtgggacagcagtgctagtgtgttcggcggaggcacccatctgaccgtcctcg

SEQ ID NO: 354 - protein, 106 aa, Canis sp.
SYVLTQLPSKNVTLKQPAHITCGGDNIGSKSVHWYQQKLGQAPVLIIYYDSSRPTGIPERFSGANSGNTATLTISGALAEDEADYYCQVWDSSASVFGGGTHLTVL

SEQ ID NO: 355 - protein, 7 aa, Canis sp.
GFTFSNY

SEQ ID NO: 356 - protein, 6 aa, Canis sp.
SGDGRS

SEQ ID NO: 357 - protein, 15 aa, Canis sp.
CAKEKYSSGWSFEYW

SEQ ID NO: 358 - protein, 11 aa, Canis sp.
GGDNIGSKSVH

SEQ ID NO: 359 - protein, 7 aa, Canis sp.
YDSSRPT

SEQ ID NO: 360 - protein, 11 aa, Canis sp.
CQVWDSSASVF

SEQ ID NO: 361 - DNA, 367 bp, Canis sp.
  source 1..367: mol_type = other DNA
gaggtgcagctggtggagtctgggggagacctgatgaagcctggggggtccctgagactttcctgtgtggcctctggattcaccttcagtagcttccccatgagttgggtccgccaggctccagggaaggggcttcagtgggtcgcatacattcacagtggtggaagtagcacaagctatgcagacgctgtgaagggccgattcaccatctccagagacaacgccaagaatacgctgtatcttcagatgaacagcctgcgagccgaggacacggccatatattactgtgcgcgtaccttccctacggtagctgctgacaactggttctactactggggccaagggaccctggtcactgtgtcctcag

SEQ ID NO: 362 - protein, 122 aa, Canis sp.
EVQLVESGGDLMKPGGSLRLSCVASGFTFSSFPMSWVRQAPGKGLQWVAYIHSGGSSTSYADAVKGRFTISRDNAKNTLYLQMNSLRAEDTAIYYCARTFPTVAADNWFYYWGQGTLVTVSS

SEQ ID NO: 363 - DNA, 322 bp, Canis sp.
  source 1..322: mol_type = other DNA
tcctatgtgctgacacagctgccatccaaaaatgtgaccctgaagcaggcggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaagtgggtattcggtgaagggacccagctgaccgtcctcg

SEQ ID NO: 364 - protein, 107 aa, Canis sp.
SYVLTQLPSKNVTLKQAAHITCGGDNIGSKSVHWYQQKLGQAPVLIIYYDSSRPTGIPERFSGANSGNTATLTISGALAEDEADYYCQVWDSSAKWVFGEGTQLTVL

SEQ ID NO: 365 - protein, 7 aa, Canis sp.
GFTFSSF

SEQ ID NO: 366 - protein, 6 aa, Canis sp.
HSGGSS

SEQ ID NO: 367 - protein, 17 aa, Canis sp.
CARTFPTVAADNWFYYW

SEQ ID NO: 368 - protein, 11 aa, Canis sp.
GGDNIGSKSVH

SEQ ID NO: 369 - protein, 7 aa, Canis sp.
YDSSRPT

SEQ ID NO: 370 - protein, 12 aa, Canis sp.
CQVWDSSAKWVF

SEQ ID NO: 371 - DNA, 367 bp, Canis sp.
  source 1..367: mol_type = other DNA
gaggtgcagctggtggagtctgggggagacctggtgaagcctggggggtccctgagactttcctgtgtggcctctggattcaccttcagtagtttccccatgagctgggtccgccaggctccagggaaggggcttcagtgggtcgcatacattaacagtggtggaagtagcacaagttatgcagacgttgtgaagggccgattcaccatctccagagacaacgccaagaacacgctgtatcttcagatgaacagcctgagagccgacgacacggccgtatattactgtgcgcgtaccttccctacggtagctactgacaactggttctactactggggccaagggaccctggtcactgtgtcctcag

SEQ ID NO: 372 - protein, 122 aa, Canis sp.
EVQLVESGGDLVKPGGSLRLSCVASGFTFSSFPMSWVRQAPGKGLQWVAYINSGGSSTSYADVVKGRFTISRDNAKNTLYLQMNSLRADDTAVYYCARTFPTVATDNWFYYWGQGTLVTVSS

SEQ ID NO: 373 - DNA, 322 bp, Canis sp.; identical to SEQ ID NO: 363
  source 1..322: mol_type = other DNA

SEQ ID NO: 374 - protein, 107 aa, Canis sp.; identical to SEQ ID NO: 364

SEQ ID NO: 375 - protein, 7 aa, Canis sp.
GFTFSSF

SEQ ID NO: 376 - protein, 6 aa, Canis sp.
NSGGSS

SEQ ID NO: 377 - protein, 17 aa, Canis sp.
CARTFPTVATDNWFYYW

SEQ ID NO: 378 - protein, 11 aa, Canis sp.
GGDNIGSKSVH

SEQ ID NO: 379 - protein, 7 aa, Canis sp.
YDSSRPT

SEQ ID NO: 380 - protein, 12 aa, Canis sp.
CQVWDSSAKWVF

SEQ ID NO: 381 - DNA, 358 bp, Canis sp.
  source 1..358: mol_type = other DNA
gaggtgcagttggtggagtctgggggagaccttgtgaaacctgaggggtccctgagactctcctgtgtggtctctggcttcaccttcagtagttatgacatgagctgggtccgccaggctccagggaaggggctgcagtgggtcgcatacattagctttgatggaaggagcacaagttacacagacgttgtgaagggccgattcaccatctccagagacaatgccaagaccacgctgtatctgcagatgaacaccctgagaattgaggacacagccgtgtattactgtgcgaagttaagttctagtggttggagtatggactattggggccatggcacctcactcttcgtgtcctcag

SEQ ID NO: 382 - protein, 119 aa, Canis sp.
EVQLVESGGDLVKPEGSLRLSCVVSGFTFSSYDMSWVRQAPGKGLQWVAYISFDGRSTSYTDVVKGRFTISRDNAKTTLYLQMNTLRIEDTAVYYCAKLSSSGWSMDYWGHGTSLFVSS

SEQ ID NO: 383 - DNA, 319 bp, Canis sp.
  source 1..319: mol_type = other DNA
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctagtgtgttcggcggaggcacccatctgaccgtcctcg

SEQ ID NO: 384 - protein, 106 aa, Canis sp.; identical to SEQ ID NO: 354

SEQ ID NO: 385 - protein, 7 aa, Canis sp.
GFTFSSY

SEQ ID NO: 386 - protein, 6 aa, Canis sp.
SFDGRS

SEQ ID NO: 387 - protein, 14 aa, Canis sp.
CAKLSSSGWSMDYW

SEQ ID NO: 388 - protein, 11 aa, Canis sp.
GGDNIGSKSVH

SEQ ID NO: 389 - protein, 7 aa, Canis sp.
YDSSRPT

SEQ ID NO: 390 - protein, 11 aa, Canis sp.
CQVWDSSASVF

SEQ ID NO: 391 - DNA, 355 bp, Canis sp.
  source 1..355: mol_type = other DNA
gaggtacaactggtggagtctgggggagacctggtgaggcctggggggtccctgagactttcctgtgtggcctctggattcaccttcaataattaccacatgagctgggtccgccaggctcctgggaaggggcttcagtgggtcgcattcattaacagtggtggaagtggtacaacctttgcagacgttgtgaggggccgattcaccatctccagagactacgccaaaaacgccctgtatcttcagatgaacagcctgagagccgaggacacggccgtatattattgtgcgagtggatctatatggactcattttgactactggggccagggaaccctggtcaccgtctcctcag

SEQ ID NO: 392 - protein, 118 aa, Canis sp.
EVQLVESGGDLVRPGGSLRLSCVASGFTFNNYHMSWVRQAPGKGLQWVAFINSGGSGTTFADVVRGRFTISRDYAKNALYLQMNSLRAEDTAVYYCASGSIWTHFDYWGQGTLVTVSS

SEQ ID NO: 393 - DNA, 322 bp, Canis sp.
  source 1..322: mol_type = other DNA
tcctttgtgctgacacagctgccatccgtgagtgtgaccctgaggcagacggcccgcatcacctgtgggggagacatcattggaagtaaaaatgtttactggtaccagcagaggctgggccaggcccctgtattgattatctatagagataacaacaggccgacagggatccctgagcgattctctggcgccagctcggggaacacggccaccctgaccatcagcggggccctggccgaggacgaggctgactattactgccaggtgtgggacaacagtaataaggctatgttcggcggaggcacccatctgaccgtcctcg

SEQ ID NO: 394 - protein, 107 aa, Canis sp.
SFVLTQLPSVSVTLRQTARITCGGDIIGSKNVYWYQQRLGQAPVLIIYRDNNRPTGIPERFSGASSGNTATLTISGALAEDEADYYCQVWDNSNKAMFGGGTHLTVL

SEQ ID NO: 395 - protein, 7 aa, Canis sp.
GFTFNNY

SEQ ID NO: 396 - protein, 6 aa, Canis sp.
NSGGSG

SEQ ID NO: 397 - protein, 13 aa, Canis sp.
CASGSIWTHFDYW

SEQ ID NO: 398 - protein, 11 aa, Canis sp.
GGDIIGSKNVY

SEQ ID NO: 399 - protein, 7 aa, Canis sp.
RDNNRPT

SEQ ID NO: 400 - protein, 12 aa, Canis sp.
CQVWDNSNKAMF

SEQ ID NO: 401 - DNA, 361 bp, Canis sp.
  source 1..361: mol_type = other DNA
gaggtgcagctggtggagtctgggggagacctggtgaagcctggggggtccctgagactttcctgtgtggcctctggattcaccttcagtagttatcacatgagctgggtccgccaggctccagggaaggggcttcagtgggtcgcatatattaccagtggtgggattaccacaagctatgcagacgctgtgaagggccgattcaccatctccagagacaacgccaagaacacgctgtatcttcagatgaacagcctgagagccgaggacacggccgtgtattactgtgcgagtgatctggttctaatattagggggggctgactactggggccagggaaccctggtcaccgtctcctcag

SEQ ID NO: 402 - protein, 120 aa, Canis sp.
EVQLVESGGDLVKPGGSLRLSCVASGFTFSSYHMSWVRQAPGKGLQWVAYITSGGITTSYADAVKGRFTISRDNAKNTLYLQMNSLRAEDTAVYYCASDLVLILGGADYWGQGTLVTVSS

SEQ ID NO: 403 - DNA, 322 bp, Canis sp.
  source 1..322: mol_type = other DNA
tcctatgagctgactcagccaccatccgtgaatgtgaccctgagtgagacggcccacatcacctgtgggggagacagcattggaaggaaatatgttcaatggatccagcagaatccaggccaggcccccgtggtgattatctataaagatagcaacaggccgacagggatccctgagcgcttctctggcgccaactcagggaacacggctaccctgaccatcagtggggccctggccgaagacgaggctgactattactgccaggtgggggacagtggtactaaggctgtgttcggcggaggcacccacctgaccgtcctcg

SEQ ID NO: 404 - protein, 107 aa, Canis sp.
SYELTQPPSVNVTLSETAHITCGGDSIGRKYVQWIQQNPGQAPVVIIYKDSNRPTGIPERFSGANSGNTATLTISGALAEDEADYYCQVGDSGTKAVFGGGTHLTVL

SEQ ID NO: 405 - protein, 7 aa, Canis sp.
GFTFSSY

SEQ ID NO: 406 - protein, 6 aa, Canis sp.
TSGGIT

SEQ ID NO: 407 - protein, 15 aa, Canis sp.
CASDLVLILGGADYW

SEQ ID NO: 408 - protein, 11 aa, Canis sp.
GGDSIGRKYVQ

SEQ ID NO: 409 - protein, 7 aa, Canis sp.
KDSNRPT

SEQ ID NO: 410 - protein, 12 aa, Canis sp.
CQVGDSGTKAVF

SEQ ID NO: 411 - DNA, 358 bp, Canis sp.; identical to SEQ ID NO: 251
  source 1..358: mol_type = other DNA

SEQ ID NO: 412 - protein, 119 aa, Canis sp.; identical to SEQ ID NO: 252

SEQ ID NO: 413 - DNA, 319 bp, Canis sp.; identical to SEQ ID NO: 253
  source 1..319: mol_type = other DNA

SEQ ID NO: 414 - protein, 106 aa, Canis sp.; identical to SEQ ID NO: 254

SEQ ID NO: 415 - protein, 7 aa, Canis sp.
GFTFNNY

SEQ ID NO: 416 - protein, 6 aa, Canis sp.
YESGST

SEQ ID NO: 417 - protein, 14 aa, Canis sp.
CASASSSSWRFDYW

SEQ ID NO: 418 - protein, 11 aa, Canis sp.
GGDSIGRKNVC

SEQ ID NO: 419 - protein, 7 aa, Canis sp.
DDRSRPS

SEQ ID NO: 420 - protein, 11 aa, Canis sp. [sequence omitted]

SEQ ID NO: 421 - DNA, 361 bp, Canis sp.
  source 1..361: mol_type = other DNA
gaggtgcagctggtggagtctgggggagacctggtgaagcctggggggtccctgagactttcctgtgtggcctctggattcaccttcagtagccaccacatgacctgggtccgccaggctccagggaaggggcttcagtgggtcgcatatattgtcagtggtggaactaacactaactatgcagacgctgtgaagggccggttcaccatctccagagacaacgccaagaacacgctgtatcttcagatgaacagcctgagagccgaggacacggccgtgtattactgtgcgagtgatctagttctagcagttggggctcctttctactggggccagggaaccctggtcaccgtctcctcag

SEQ ID NO: 422 - protein, 120 aa, Canis sp.
EVQLVESGGDLVKPGGSLRLSCVASGFTFSSHHMTWVRQAPGKGLQWVAYIVSGGTNTNYADAVKGRFTISRDNAKNTLYLQMNSLRAEDTAVYYCASDLVLAVGAPFYWGQGTLVTVSS

SEQ ID NO: 423 - DNA, 322 bp, Canis sp.
  source 1..322: mol_type = other DNA
tcctatgagctgactcagccaccatccgtgaatgtgaccctgagggagacggcccacatcacctgtgggggagacagcattggaagaaaatatgttcaatggatccagcagaatccaggccaggcccccgtggtaattatctataaagacatcaacaggccgacagggatccctgagcgattctctggcgccaactcagggaacacggctaccctgaccatcagtggggccctggccgaagacgaggctgactattactgccaggtgggggacagtggtactaaggcggtattcggtgaagggacccagctgaccgtcctcg

SEQ ID NO: 424 - protein, 107 aa, Canis sp.
SYELTQPPSVNVTLRETAHITCGGDSIGRKYVQWIQQNPGQAPVVIIYKDINRPTGIPERFSGANSGNTATLTISGALAEDEADYYCQVGDSGTKAVFGEGTQLTVL

SEQ ID NO: 425 - protein, 7 aa, Canis sp.
GFTFSSH

SEQ ID NO: 426 - protein, 6 aa, Canis sp.
VSGGTN

SEQ ID NO: 427 - protein, 15 aa, Canis sp.
CASDLVLAVGAPFYW

SEQ ID NO: 428 - protein, 11 aa, Canis sp.
GGDSIGRKYVQ

SEQ ID NO: 429 - protein, 7 aa, Canis sp.
KDINRPT

SEQ ID NO: 430 - protein, 12 aa, Canis sp.
CQVGDSGTKAVF

SEQ ID NO: 431 - DNA, 361 bp, Canis sp.
  source 1..361: mol_type = other DNA
gaggtgcaactggtggagtctgggggagacctggtgaagcctggggggtccctgagactttcctgtgtggcctctggattcaccttcagaacctaccacatgagctgggtccgccaggctccagggaaggggcttcagtgggtcgcatacattacccgtgatggaattagcacaacctatacagacgctgtgaagggccgattcaccatctccagagacaacgccaagaacactctgtttcttcagatgaacagcctgagagccgatgacacggccgtgtattactgtgcgagtgatctagttctagtacttggggctcctttctactggggccagggaaccctggtcaccgtctcctcag

SEQ ID NO: 432 - protein, 120 aa, Canis sp.
EVQLVESGGDLVKPGGSLRLSCVASGFTFRTYHMSWVRQAPGKGLQWVAYITRDGISTTYTDAVKGRFTISRDNAKNTLFLQMNSLRADDTAVYYCASDLVLVLGAPFYWGQGTLVTVSS

SEQ ID NO: 433 - DNA, 322 bp, Canis sp.
  source 1..322: mol_type = other DNA
tcctatgagctgactcagccaccatccgtgaatgtgaccctgggggagacggcccacatcacctgtgggggagacagcattggaagaaaatatgttcaatggatccagcagaatccaggccaggcccccgtggtgattatctataaagatagcaacaggccgacagggatccctgagcgattctctggcgccaactcagggaacacggctaccctgaccatcagtggggccctggccgaagacgaggctgactattactgccaggtgggggacagtggtactaaggcggtattcggtgaagggacccagctgaccgtcctcg

SEQ ID NO: 434 - protein, 107 aa, Canis sp.
SYELTQPPSVNVTLGETAHITCGGDSIGRKYVQWIQQNPGQAPVVIIYKDSNRPTGIPERFSGANSGNTATLTISGALAEDEADYYCQVGDSGTKAVFGEGTQLTVL

SEQ ID NO: 435 - protein, 7 aa, Canis sp.
GFTFRTY

SEQ ID NO: 436 - protein, 6 aa, Canis sp.
TRDGIS

SEQ ID NO: 437 - protein, 15 aa, Canis sp.
CASDLVLVLGAPFYW

SEQ ID NO: 438 - protein, 11 aa, Canis sp.
GGDSIGRKYVQ

SEQ ID NO: 439 - protein, 7 aa, Canis sp.
KDSNRPT

SEQ ID NO: 440 - protein, 12 aa, Canis sp.
CQVGDSGTKAVF

SEQ ID NO: 441 - DNA, 355 bp, Canis sp.
  source 1..355: mol_type = other DNA
gaggtgcagctggtggagtctgggggagaccttgtgaaacctgaggggtccctgagactctcctgtgtggtctctggcttcactttcagtaattacgacatgagctgggtccgccaggctccagggaaggggctgcagtgggtcgcattcattagcaatgatgggaggatcacaggttacacagacgctgtggagggccgattcaccatctccagagacaacgccaagaacacgctgtatctgcagatgaacagcctgagaactgaggacacagccgtgtattactgtgcgaaggagactagtcgttgggggtttgactactggggccagggaaccctggtcaccgtctcctcag

SEQ ID NO: 442 - protein, 118 aa, Canis sp.
EVQLVESGGDLVKPEGSLRLSCVVSGFTFSNYDMSWVRQAPGKGLQWVAFISNDGRITGYTDAVEGRFTISRDNAKNTLYLQMNSLRTEDTAVYYCAKETSRWGFDYWGQGTLVTVSS

SEQ ID NO: 443 - DNA, 319 bp, Canis sp.
  source 1..319: mol_type = other DNA
tcctatgtgctgacacagctgccatccgtgagtgtgaccctgaggcagacggcccgcatcacctgtgggggagacagcattggaagtaaaaatgtttactggcaccagcagaagctgggccaggcccctgtactgattatctatgatgatagcagcaggccgtcagggatccctgagcgattctccggcgccaattcggggaatacggccaccctgaccatcagcggggccctggccgaggatgaggctgactattactgtcaggtgtgggacagtagtactagtgtgttcggcggaggcacccatctgaccgtcctcg

SEQ ID NO: 444 - protein, 106 aa, Canis sp.
SYVLTQLPSVSVTLRQTARITCGGDSIGSKNVYWHQQKLGQAPVLIIYDDSSRPSGIPERFSGANSGNTATLTISGALAEDEADYYCQVWDSSTSVFGGGTHLTVL

SEQ ID NO: 445 - protein, 7 aa, Canis sp.
GFTFSNY

SEQ ID NO: 446 - protein, 6 aa, Canis sp.
SNDGRI

SEQ ID NO: 447 - protein, 13 aa, Canis sp.
CAKETSRWGFDYW

SEQ ID NO: 448 - protein, 11 aa, Canis sp.
GGDSIGSKNVY

SEQ ID NO: 449 - protein, 7 aa, Canis sp.
DDSSRPS

SEQ ID NO: 450 - protein, 11 aa, Canis sp.
CQVWDSSTSVF

SEQ ID NO: 451 - DNA, 355 bp, Canis sp.
  source 1..355: mol_type = other DNA
gaggttaagctggtggagtctgggggagaccttgtgaaacctgaggggtccctgagactctcctgtgtggtctctggcttcacctttagtaggtacggcatgagctgggtccgccaggctccagggaaggggctgcagtgggtcgcgtacattagcagtgatggaaggagctcaagttacacagacgctgtgcagggccgattcaccatctccagcgacaatgccaagaacacgctgtatctgcagatgaacagcctgacaactgaggacacagccgtgtattactgtgcgaagttggggcgcggatttaattttaacttctggggccagggaaccctggtcaccgtctcctcag

SEQ ID NO: 452 - protein, 118 aa, Canis sp.
EVKLVESGGDLVKPEGSLRLSCVVSGFTFSRYGMSWVRQAPGKGLQWVAYISSDGRSSSYTDAVQGRFTISSDNAKNTLYLQMNSLTTEDTAVYYCAKLGRGFNFNFWGQGTLVTVSS

SEQ ID NO: 453 - DNA, 319 bp, Canis sp.; identical to SEQ ID NO: 383
  source 1..319: mol_type = other DNA

SEQ ID NO: 454 - protein, 106 aa, Canis sp.; identical to SEQ ID NO: 354

SEQ ID NO: 455 - protein, 7 aa, Canis sp.
GFTFSRY

SEQ ID NO: 456 - protein, 6 aa, Canis sp.
SSDGRS

SEQ ID NO: 457 - protein, 13 aa, Canis sp.
CAKLGRGFNFNFW

SEQ ID NO: 458 - protein, 11 aa, Canis sp.
GGDNIGSKSVH

SEQ ID NO: 459 - protein, 7 aa, Canis sp.
YDSSRPT

SEQ ID NO: 460 - protein, 11 aa, Canis sp.
CQVWDSSASVF

SEQ ID NO: 461 - DNA, 355 bp, Canis sp.
  source 1..355: mol_type = other DNA
gaggtgcagctggtggagtctgggggagacctggtgaagcctggggggtccctgagactttcctgtgtggcctctggattcaccttcagtagctaccacatgagctgggtccgccaggctccagggaagggacttcagtgggtcgcattcattaacagtgctgaaagtagcacaacctatgcagacgctgtgaagggccgattcaccatctccagagacaacgccaagaacacgctgtatcttcagatgaacagcctgagagccgaggacacggccgtatattactgtgcgggtggatatatatggacccattttgactactggggccagggaaccctggtcaccgtctcctcag

SEQ ID NO: 462 - protein, 118 aa, Canis sp.
EVQLVESGGDLVKPGGSLRLSCVASGFTFSSYHMSWVRQAPGKGLQWVAFINSAESSTTYADAVKGRFTISRDNAKNTLYLQMNSLRAEDTAVYYCAGGYIWTHFDYWGQGTLVTVSS

SEQ ID NO: 463 - DNA, 319 bp, Canis sp.
  source 1..319: mol_type = other DNA
tcctatgtgctgacacagctgccatccgtgagtgtgaccctgaggcagacggcccgcatcacctgtgggggagacatcattggaagtaaaagtgtttactggtaccagcagaatctgggccaggcccctgtactgattatctatagagataccaacaggccgacagggatccctgagcgattctctggcgccagctcggggaacacggccaccctgaccatcagcggggccctggccgaggacgaggctgactattactgccaggtgtgggacagcagtactagtgtgttcggcggaggcacccatctgaccgtcctcg

SEQ ID NO: 464 - protein, 106 aa, Canis sp.
SYVLTQLPSVSVTLRQTARITCGGDIIGSKSVYWYQQNLGQAPVLIIYRDTNRPTGIPERFSGASSGNTATLTISGALAEDEADYYCQVWDSSTSVFGGGTHLTVL

SEQ ID NO: 465 - protein, 7 aa, Canis sp.
GFTFSSY

SEQ ID NO: 466 - protein, 6 aa, Canis sp.
NSAESS

SEQ ID NO: 467 - protein, 13 aa, Canis sp.
CAGGYIWTHFDYW

SEQ ID NO: 468 - protein, 11 aa, Canis sp.
GGDIIGSKSVY

SEQ ID NO: 469 - protein, 7 aa, Canis sp.
RDTNRPT

SEQ ID NO: 470 - protein, 11 aa, Canis sp.
CQVWDSSTSVF

SEQ ID NO: 471 - DNA, 355 bp, Canis sp.
  source 1..355: mol_type = other DNA
gaggtgcacctggtggagtctgggggagacctggtgaagcctggggggtccctgagactttcctgtgtggcctctggattcaccttcagtaactaccacatgagctgggtccgccaggctccagggaaggggcttcagtgggtcgcattcattaacagtggtggaagtggcacaagctatgcagacgctgtgaagggccgattcaccatctccagagactacgccaagagcacgctatatcttcagatgaacagcctgagagccgaggacacggccgtctattactgtgcgagtggatatatatggacccattttgactactggggccagggaaccctggtcaccgtctcctcag

SEQ ID NO: 472 - protein, 118 aa, Canis sp.
EVHLVESGGDLVKPGGSLRLSCVASGFTFSNYHMSWVRQAPGKGLQWVAFINSGGSGTSYADAVKGRFTISRDYAKSTLYLQMNSLRAEDTAVYYCASGYIWTHFDYWGQGTLVTVSS

SEQ ID NO: 473 - DNA, 319 bp, Canis sp.
  source 1..319: mol_type = other DNA
tcctatgtgctgacacagctgccatccgtgagtgtgaccctgaggcagacggcccgcatcacctgtgggggagacatcattggaagtaaaaatgtttactggtaccagcagaagctgggccaggcccctatactgattatctatagagatagcaacaggccgacagggatccctgagcgattctctggcgccagctcggggaacacggccaccctgaccatcagcggggccctggccgaggacgaggctgactattactgccaggtgtgggacagcagtactagtgtgttcggcggaggcacccatctgaccgtcctcg

SEQ ID NO: 474 - protein, 106 aa, Canis sp.
SYVLTQLPSVSVTLRQTARITCGGDIIGSKNVYWYQQKLGQAPILIIYRDSNRPTGIPERFSGASSGNTATLTISGALAEDEADYYCQVWDSSTSVFGGGTHLTVL

SEQ ID NO: 475 - protein, 7 aa, Canis sp.
GFTFSNY

SEQ ID NO: 476 - protein, 6 aa, Canis sp.
NSGGSG

SEQ ID NO: 477 - protein, 13 aa, Canis sp.
CASGYIWTHFDYW

SEQ ID NO: 478 - protein, 11 aa, Canis sp.
GGDIIGSKNVY

SEQ ID NO: 479 - protein, 7 aa, Canis sp.
RDSNRPT

SEQ ID NO: 480 - protein, 11 aa, Canis sp.
CQVWDSSTSVF

SEQ ID NO: 481 - DNA, 355 bp, Canis sp.
  source 1..355: mol_type = other DNA
gaggtgcagttggtggagtctgggggagacctggtgaagcctggggggtccctgagactttcctgtgtggcctctggattcaccttcaatacctaccacatgaactgggtccgccaggctccagggaaggggcttcactgggtcgcattcattaacagcggtgggactggcacaggctattcagacactgtgaagggccgattcaccatctccagagactacgccaagaatacactctatcttcagatgaatagtctgagagccgaagacacggccgtttattattgtgtgagtggatctatatggactcattttgactactggggccagggaaccctggtcaccgtctcctcag

SEQ ID NO: 482 - protein, 118 aa, Canis sp.
EVQLVESGGDLVKPGGSLRLSCVASGFTFNTYHMNWVRQAPGKGLHWVAFINSGGTGTGYSDTVKGRFTISRDYAKNTLYLQMNSLRAEDTAVYYCVSGSIWTHFDYWGQGTLVTVSS

SEQ ID NO: 483 - DNA, 322 bp, Canis sp.
  source 1..322: mol_type = other DNA
tcctatgtgctgacacagctgccatccgtgagtatgaccctgaggcagacggcccacatcacctgtgggggagacatcattggaagtaaaagtgtttactggtaccagcagaagctgggccaggcccctgtactgattatttatagagataccaacaggccgacagggatccctgagcgattctctggcgccagctcggggaacacggccaccctgaccatcagcggggccctggccgaggacgaggctgactattactgccaggtttgggacagcaatactcaggctgtgttcggcggaggcacccatctgaccgtcctcg

SEQ ID NO: 484 - protein, 107 aa, Canis sp.
SYVLTQLPSVSMTLRQTAHITCGGDIIGSKSVYWYQQKLGQAPVLIIYRDTNRPTGIPERFSGASSGNTATLTISGALAEDEADYYCQVWDSNTQAVFGGGTHLTVL

SEQ ID NO: 485 - protein, 7 aa, Canis sp.
GFTFNTY

SEQ ID NO: 486 - protein, 6 aa, Canis sp.
NSGGTG

SEQ ID NO: 487 - protein, 13 aa, Canis sp.
CVSGSIWTHFDYW

SEQ ID NO: 488 - protein, 11 aa, Canis sp.
GGDIIGSKSVY

SEQ ID NO: 489 - protein, 7 aa, Canis sp.
RDTNRPT

SEQ ID NO: 490 - protein, 12 aa, Canis sp.
CQVWDSNTQAVF

SEQ ID NO: 491 - DNA, 355 bp, Canis sp.
  source 1..355: mol_type = other DNA
gaggtgcagctggtggagtctgggggagacctggtgaagcctggggggtccctgagactttcctgtgtggcctctggattcaccttcaataacttccacatgagttgggtccgccaggctccagggaaggggcttcagtgggtcgcattcattaacagtgatggaggtaatagaaactatgcagacgttgtgaagggccgagtcaccatctccagagactacgccaagaatacgctgtatcttcagataaacagtctgagagccgaggacacggccgtgtattactgtgcgggtggatctatatggactcactttgactactggggccagggaaccctggtcaccgtctcctcag

SEQ ID NO: 492 - protein, 118 aa, Canis sp.
EVQLVESGGDLVKPGGSLRLSCVASGFTFNNFHMSWVRQAPGKGLQWVAFINSDGGNRNYADVVKGRVTISRDYAKNTLYLQINSLRAEDTAVYYCAGGSIWTHFDYWGQGTLVTVSS

SEQ ID NO: 493 - DNA, 322 bp, Canis sp.
  source 1..322: mol_type = other DNA
tcctatgtgttgacacagctgccatccatgagtgtgaccctgaggcagacggcccgcatcacctgtgagggagacatcattgcaagtaaaagaatttactggtaccaacagaagctgggccaggtccctgtactgcttatctatgctgatagcagcaggccgtcagggatccctgaacgattctccggcgccagctcggggaacacagccaccctgaccatcagcggggccctggccgaggacgaggctgactattactgccaggtgtgggacaacaatgctaatagtgtgttcggcggaggcacccatctgaccgtcctcg

SEQ ID NO: 494 - protein, 107 aa, Canis sp.
SYVLTQLPSMSVTLRQTARITCEGDIIASKRIYWYQQKLGQVPVLLIYADSSRPSGIPERFSGASSGNTATLTISGALAEDEADYYCQVWDNNANSVFGGGTHLTVL

SEQ ID NO: 495 - protein, 7 aa, Canis sp.
GFTFNNF

SEQ ID NO: 496 - protein, 6 aa, Canis sp.
NSDGGN

SEQ ID NO: 497 - protein, 13 aa, Canis sp.
CAGGSIWTHFDYW

SEQ ID NO: 498 - protein, 11 aa, Canis sp.
EGDIIASKRIY

SEQ ID NO: 499 - protein, 7 aa, Canis sp.
ADSSRPS

SEQ ID NO: 500 - protein, 12 aa, Canis sp.
CQVWDNNANSVF

SEQ ID NO: 501 - DNA, 355 bp, Canis sp.
  source 1..355: mol_type = other DNA
gaggtacagttggtggaatctgggggagacctggtgaagcctggggggtccctgagactttcctgtgtggcctctggattcaccttcaatacctaccacatgaactgggtccgccaggctccagggaagggacttcactgggtcgcattcattaacagcggtggaggtggcacaagctatgcagacactgtgcagggccgattcaccatctccagagactacgccaagaacgcactctatcttcagatgaacagcctgagagccgaggacacggccgtttattactgtgcgagtggatctatatggactcattttgactactggggccagggaaccctggtcaccgtctcctcag

SEQ ID NO: 502 - protein, 118 aa, Canis sp.
EVQLVESGGDLVKPGGSLRLSCVASGFTFNTYHMNWVRQAPGKGLHWVAFINSGGGGTSYADTVQGRFTISRDYAKNALYLQMNSLRAEDTAVYYCASGSIWTHFDYWGQGTLVTVSS

SEQ ID NO: 503 - DNA, 322 bp, Canis sp.
  source 1..322: mol_type = other DNA
tcctatgtgctgacacagctgccattcatgcgtgtgaccctgagtcagacggcccgcatcacctgtgagggagacaacattggaagtaaaagagttttctggtaccagcagaagccgggccaggtccctgtagtgattattcatgatgatagcagcaggccgtcagggatccctgagcgattctccggcgccaattcggggaacacagccaccctgaccatcagcggggccctggccgaggacgaggctgcctattactgccaggtgtgggacagcagtactaatagtgtgttcggcgggggcacccatctgaccgtcctcg

SEQ ID NO: 504 - protein, 107 aa, Canis sp.
SYVLTQLPFMRVTLSQTARITCEGDNIGSKRVFWYQQKPGQVPVVIIHDDSSRPSGIPERFSGANSGNTATLTISGALAEDEAAYYCQVWDSSTNSVFGGGTHLTVL

SEQ ID NO: 505 - protein, 7 aa, Canis sp.
GFTFNTY

SEQ ID NO: 506 - protein, 6 aa, Canis sp.
NSGGGG

SEQ ID NO: 507 - protein, 13 aa, Canis sp.
CASGSIWTHFDYW

SEQ ID NO: 508 - protein, 11 aa, Canis sp.
EGDNIGSKRVF

SEQ ID NO: 509 - protein, 7 aa, Canis sp.
DDSSRPS

SEQ ID NO: 510 - protein, 12 aa, Canis sp.
CQVWDSSTNSVF

SEQ ID NO: 511 - DNA, 355 bp, Canis sp.
  source 1..355: mol_type = other DNA
gaggtgcagctggtggagtctgggggagacctggtgaggcctggggggtccctgagactttcctgtgtggcctctggattcaccttcagtaactaccacatgaattgggtccgccaggctccagggaaggggcttcagtgggtcgcattcattaacagtggtggaggtggcacatcctatgcagacgttgtgaggggccgattcaccatctccagagactacgccaaaaacacattgtatcttcagatgaacagcctgagcgccgaggacacggccgtatattactgtgcgagtggatctatatggactcattttgactactggggccagggaaccctggtcaccgtctcctcag

SEQ ID NO: 512 - protein, 118 aa, Canis sp.
EVQLVESGGDLVRPGGSLRLSCVASGFTFSNYHMNWVRQAPGKGLQWVAFINSGGGGTSYADVVRGRFTISRDYAKNTLYLQMNSLSAEDTAVYYCASGSIWTHFDYWGQGTLVTVSS

SEQ ID NO: 513 - DNA, 322 bp, Canis sp.
  source 1..322: mol_type = other DNA
tcctatgtgctgacacagctgacatccatgagtgtgaccctgaggcaggcggcccgcttcacctgtgagagagacaccattggaagtaaaagagtttactggtaccagcagaggctgggccaggtccctgtcctgattatctatgattataacatcaggccgtcagggatccctgagcgactctccggcgcctattcggggaacacagccgccctgaccatcagcggggccctggccgaggacgaggctgactatttctgccaggtgtgggacagcagtgctaatagtgtgttcggcggaggcacccatctgaccgtcctcg

SEQ ID NO: 514 - protein, 107 aa, Canis sp.
SYVLTQLTSMSVTLRQAARFTCERDTIGSKRVYWYQQRLGQVPVLIIYDYNIRPSGIPERLSGAYSGNTAALTISGALAEDEADYFCQVWDSSANSVFGGGTHLTVL

SEQ ID NO: 515 - protein, 7 aa, Canis sp.
GFTFSNY

SEQ ID NO: 516 - protein, 6 aa, Canis sp.
NSGGGG

SEQ ID NO: 517 - protein, 13 aa, Canis sp.
CASGSIWTHFDYW

SEQ ID NO: 518 - protein, 11 aa, Canis sp.
ERDTIGSKRVY

SEQ ID NO: 519 - protein, 7 aa, Canis sp.
DYNIRPS

SEQ ID NO: 520 - protein, 12 aa, Canis sp.
CQVWDSSANSVF

SEQ ID NO: 521 - DNA, 355 bp, Canis sp.
  source 1..355: mol_type = other DNA
gaggtgcagttggtggagtctgggggagacctggtgaagccgggggggtccctgagactttcctgtgtggcctctggattcaccttcaataacctccacatgagttgggtccgccaggctccagggaaggggcttcagtgggtcgcattcattaacagtgatggacgtaatagaaactatgcagacgctgtgaagggccgagtcaccatctccagagactacgccaagaatacgctgtatcttcagataaacagtctgagagccgaagacacggccgtgtattactgtacgggtggatctatatggactcactttgactactggggccagggaaccctggtcaccgtctcctcag

SEQ ID NO: 522 - protein, 118 aa, Canis sp.
EVQLVESGGDLVKPGGSLRLSCVASGFTFNNLHMSWVRQAPGKGLQWVAFINSDGRNRNYADAVKGRVTISRDYAKNTLYLQINSLRAEDTAVYYCTGGSIWTHFDYWGQGTLVTVSS

SEQ ID NO: 523 - DNA, 322 bp, Canis sp.
  source 1..322: mol_type = other DNA
tcctatgtgttgacacagctgccatccatgagtgtgaccctgaggcagacggcccgcatcacctgtgagggagacatcattgcaagtaaaagagtttactggtaccaacagaagctgggccaggtccctgtactgtttatctatgctgataacagcaggccgtcagggatccctgaacgattctccggcgccagctcggggaacacagccaccctgaccatcagcggggccctggccgaggacgaggctgactattactgccaggtgtgggacagcaatgctaatagtgtgttcggcggaggcacccatctgaccgtcctcg

SEQ ID NO: 524 - protein, 107 aa, Canis sp.
SYVLTQLPSMSVTLRQTARITCEGDIIASKRVYWYQQKLGQVPVLFIYADNSRPSGIPERFSGASSGNTATLTISGALAEDEADYYCQVWDSNANSVFGGGTHLTVL

SEQ ID NO: 525 - protein, 7 aa, Canis sp.
GFTFNNL

SEQ ID NO: 526 - protein, 6 aa, Canis sp.
NSDGRN

SEQ ID NO: 527 - protein, 13 aa, Canis sp.
CTGGSIWTHFDYW

SEQ ID NO: 528 - protein, 11 aa, Canis sp.
EGDIIASKRVY

SEQ ID NO: 529 - protein, 7 aa, Canis sp.
ADNSRPS

SEQ ID NO: 530 - protein, 12 aa, Canis sp.
CQVWDSNANSVF

SEQ ID NO: 531 - DNA, 355 bp, Canis sp.
  source 1..355: mol_type = other DNA
gaggtgcagctggtggagtctgggggagacctggtgaagcctggggggtccctgagactttcctgtgtggcctctggattcaccttcagtagctaccacatgagctgggtccgccaggctccagggaaggggcttcagtgggtcgcattcattaacagtggtggaggtggcacaaactatgcagacgctgtgaagggccgattcaccatctccagagactacgccaagaatacgctgtatcttcagatgaacagcctgagagccgaggacacggccgtgtattactgtgtgggtggatctatatggactcattttgactactggggccagggaaccctggtcaccgtctcctcag

SEQ ID NO: 532 - protein, 118 aa, Canis sp.
EVQLVESGGDLVKPGGSLRLSCVASGFTFSSYHMSWVRQAPGKGLQWVAFINSGGGGTNYADAVKGRFTISRDYAKNTLYLQMNSLRAEDTAVYYCVGGSIWTHFDYWGQGTLVTVSS

SEQ ID NO: 533 - DNA, 322 bp, Canis sp.
  source 1..322: mol_type = other DNA
tcctatgtgctgacacagctgccatccatgagtgtgaccctgaggcagacggcccgcatcacctgtgagggagacatcattgcaagtaaaagagtttactggtaccaacagaagctgggccaggtccctgtactgcttatctatggtgatagcagcaggccgtcagggatccctgagcgattctccggcgccaactcggggaacacagccaccctgaccatcagcggggccctggccgaggacgaggctgactattactgccaggtgtgggacagcggtactaatagtgtgttcggcggaggcacccatctgaccgtcctcg

SEQ ID NO: 534 - protein, 107 aa, Canis sp.
SYVLTQLPSMSVTLRQTARITCEGDIIASKRVYWYQQKLGQVPVLLIYGDSSRPSGIPERFSGANSGNTATLTISGALAEDEADYYCQVWDSGTNSVFGGGTHLTVL

SEQ ID NO: 535 - protein, 7 aa, Canis sp.
GFTFSSY

SEQ ID NO: 536 - protein, 6 aa, Canis sp.
NSGGGG

SEQ ID NO: 537 - protein, 13 aa, Canis sp.
CVGGSIWTHFDYW

SEQ ID NO: 538 - protein, 11 aa, Canis sp.
EGDIIASKRVY

SEQ ID NO: 539 - protein, 7 aa, Canis sp.
GDSSRPS

SEQ ID NO: 540 - protein, 12 aa, Canis sp.
CQVWDSGTNSVF

SEQ ID NO: 541 - DNA, 355 bp, Canis sp.
  source 1..355: mol_type = other DNA
gacatacatctggtggagtctgggggagacctggtgaggcctggggggtccctgagactttcctgtgtggcctctggattcaccttcagtaacctccacatgaactgggtccgccaggctccagggaaggggcttcagtgggtcgcattcattaacagtggtggaagtggcacaacctatgcagacgttgtgaggggccgattcaccatctccagagactacgccaaaaacacactgtatcttcagatgaatagcctgagagccgacgacacggccgtatattactgtgcgagtggatctatatggactcattttgactactggggccagggaaccctggtcaccgtctcctcag

SEQ ID NO: 542 - protein, 118 aa, Canis sp.
DIHLVESGGDLVRPGGSLRLSCVASGFTFSNLHMNWVRQAPGKGLQWVAFINSGGSGTTYADVVRGRFTISRDYAKNTLYLQMNSLRADDTAVYYCASGSIWTHFDYWGQGTLVTVSS

SEQ ID NO: 543 - DNA, 322 bp, Canis sp.
  source 1..322: mol_type = other DNA
tcctttgtgttgacacagctgccatccgtgagtgtgaccctgaggcagacggcccgcatcacctgtgggggagacagtattggaagtaaaagtgtttactggtaccagcagaagttgggccaggcccctgtactgattatctatagagatagcaacaggccgacagggatccctgagcgaatctctggcgccagctcggggaacacggccaccctgaccatcagcggggccctggccgaggacgaggctgactattactgccaggtgtgggacagcagtaataaggctatgttcggcggaggcacccatctgaccgtcctcg

SEQ ID NO: 544 - protein, 107 aa, Canis sp.
SFVLTQLPSVSVTLRQTARITCGGDSIGSKSVYWYQQKLGQAPVLIIYRDSNRPTGIPERISGASSGNTATLTISGALAEDEADYYCQVWDSSNKAMFGGGTHLTVL

SEQ ID NO: 545 - protein, 7 aa, Canis sp.
GFTFSNL

SEQ ID NO: 546 - protein, 6 aa, Canis sp.
NSGGSG

SEQ ID NO: 547 - protein, 13 aa, Canis sp.
CASGSIWTHFDYW

SEQ ID NO: 548 - protein, 11 aa, Canis sp.
GGDSIGSKSVY

SEQ ID NO: 549 - protein, 7 aa, Canis sp.
RDSNRPT

SEQ ID NO: 550 - protein, 12 aa, Canis sp.
CQVWDSSNKAMF

SEQ ID NO: 551 - DNA, 355 bp, Canis sp.
  source 1..355: mol_type = other DNA
gcggtgcagctggtggagtctgggggagacctggtgaggcctggggggtccctgagactttcctgtgtggcctctggattcaccttcagtagttaccacatgaactgggtccgccaggctcctgggaaggggcttcagtgggtcgcattcattaacagtggaggaagtagtacaacctatgcagacgttgtcaggggccgattcaccatctccagagactacgccaaaaacacgctatttcttcagatgaacagcctgagagccgaggacacggccgtgtattattgtgcgattggatctatatggacacattttgactactggggccagggaaccctggtcaccgtctcctcag

SEQ ID NO: 552 - protein, 118 aa, Canis sp.
AVQLVESGGDLVRPGGSLRLSCVASGFTFSSYHMNWVRQAPGKGLQWVAFINSGGSSTTYADVVRGRFTISRDYAKNTLFLQMNSLRAEDTAVYYCAIGSIWTHFDYWGQGTLVTVSS

SEQ ID NO: 553 - DNA, 322 bp, Canis sp.
  source 1..322: mol_type = other DNA
tcctttgtgctgacacagctgccatccgtgagtgtgaccctgaggcagacggcccgcatcacctgtgggggagacatcattggaagtaaaaatgtttactggtaccagcagaggctgggccaggcccctgtattgattctctatagagataccaacaggccgacagggatccctgagcgattctctggcgccagctcggggaacacggccaccctgaccatcagcggggccctggccgaggacgaggctgactattactgccaggtgtgggacaacagtaataaggctatgttcggcggaggcacccatctgaccgtcctcg

SEQ ID NO: 554 - protein, 107 aa, Canis sp.
SFVLTQLPSVSVTLRQTARITCGGDIIGSKNVYWYQQRLGQAPVLILYRDTNRPTGIPERFSGASSGNTATLTISGALAEDEADYYCQVWDNSNKAMFGGGTHLTVL

SEQ ID NO: 555 - protein, 7 aa, Canis sp.
GFTFSSY

SEQ ID NO: 556 - protein, 6 aa, Canis sp.
NSGGSS

SEQ ID NO: 557 - protein, 13 aa, Canis sp.
CAIGSIWTHFDYW

SEQ ID NO: 558 - protein, 11 aa, Canis sp.
GGDIIGSKNVY

SEQ ID NO: 559 - protein, 7 aa, Canis sp.
RDTNRPT

SEQ ID NO: 560 - protein, 12 aa, Canis sp.
CQVWDNSNKAMF

SEQ ID NO: 561 - DNA, 355 bp, Canis sp.
  source 1..355: mol_type = other DNA
gaggtacagttggtggagtctgggggagacctggtgaagcctggggggtccctgagactttcctgtgtggcctctggattcaccttcaatacttaccacatgaactgggtccgccaggctccagggaaggggcttcactgggtcgcatttattaacagcggtggaagtgacacaagctatgcagacactgtgcagggccggttcaccatctccagagactacgccaagaacacactctatcttcagatgaacagcctgagagccgaggacacggccgtgtattactgtgcgagtggatatatatggactcattttgactactggggccagggaaccctagtcaccgtctcctcag

SEQ ID NO: 562 - protein, 118 aa, Canis sp.
EVQLVESGGDLVKPGGSLRLSCVASGFTFNTYHMNWVRQAPGKGLHWVAFINSGGSDTSYADTVQGRFTISRDYAKNTLYLQMNSLRAEDTAVYYCASGYIWTHFDYWGQGTLVTVSS

SEQ ID NO: 563 - DNA, 322 bp, Canis sp.
  source 1..322: mol_type = other DNA
tcctatgtgctgacacagctgccatccgtgagtgtgaccctgaggcagacggcccacatcacctgtgggggagacatcattggaagtaaaagtgtttactggtaccagcagaagctgggccaggcccctgtaatgattatctatagagataacaacaggccgacagggatccctgagcgattctctggcgccagctcggggaacacggccaccctgaccatcagcgggtccctggccgaggacgaggctgactattactgccaggtttgggacagcaggactcaggctttgttcggcggaggcacccatctgaccgtcctcg

SEQ ID NO: 564 - protein, 107 aa, Canis sp.
SYVLTQLPSVSVTLRQTAHITCGGDIIGSKSVYWYQQKLGQAPVMIIYRDNNRPTGIPERFSGASSGNTATLTISGSLAEDEADYYCQVWDSRTQALFGGGTHLTVL

SEQ ID NO: 565 - protein, 7 aa, Canis sp.
GFTFNTY

SEQ ID NO: 566 - protein, 6 aa, Canis sp.
NSGGSD

SEQ ID NO: 567 - protein, 13 aa, Canis sp.
CASGYIWTHFDYW

SEQ ID NO: 568 - protein, 11 aa, Canis sp.
GGDIIGSKSVY

SEQ ID NO: 569 - protein, 7 aa, Canis sp.
RDNNRPT

SEQ ID NO: 570 - protein, 12 aa, Canis sp.
CQVWDSRTQALF

SEQ ID NO: 571 - DNA, 355 bp, Canis sp.
  source 1..355: mol_type = other DNA
gaggtgcagctggtggagtctgggggagacctggtgaggcctggggggtccctgagactttcctgtgtggcctctggaatcaccttcagtagttaccacatgagttgggtccgccaggctcctgggaaggggcttcagtgggtcgcactcataaacagtggtggaagtagtataacctatgcagacgttgtgaggggccgattcaccatctccagagactacgccaaaaacacgctgtatcttcagatgaacagcctgagagccgaggacacggccgtgtattattgtgcgagtggatctatatggactcattttgactattggggccaggggacccgggtcaccgtctcctcag

SEQ ID NO: 572 - protein, 118 aa, Canis sp.
EVQLVESGGDLVRPGGSLRLSCVASGITFSSYHMSWVRQAPGKGLQWVALINSGGSSITYADVVRGRFTISRDYAKNTLYLQMNSLRAEDTAVYYCASGSIWTHFDYWGQGTRVTVSS

SEQ ID NO: 573 - DNA, 322 bp, Canis sp.
  source 1..322: mol_type = other DNA
tcctttgtgctgacacagctgccatccgtgagtgtgaccctgaggcagacggcccgcatcacctgtgggggagacatcattggaagtaaaaatgtttactggtatcaacagaggctgggccaggcccctgtattgattatctatagagatagcaacaggccgacagggatccctgagcgattctctggcgccagctcggggaacacggccaccctgaccatcagcggggccctggccgaggacgaggctgactattactgccaggtgtgggacaacagtaataaggctatgttcggcggaggcacccatctgaccgtcctcg

SEQ ID NO: 574 - protein, 107 aa, Canis sp.
SFVLTQLPSVSVTLRQTARITCGGDIIGSKNVYWYQQRLGQAPVLIIYRDSNRPTGIPERFSGASSGNTATLTISGALAEDEADYYCQVWDNSNKAMFGGGTHLTVL

SEQ ID NO: 575 - protein, 7 aa, Canis sp.
GITFSSY

SEQ ID NO: 576 - protein, 6 aa, Canis sp.
NSGGSS

SEQ ID NO: 577 - protein, 13 aa, Canis sp.
CASGSIWTHFDYW

SEQ ID NO: 578 - protein, 11 aa, Canis sp.
GGDIIGSKNVY

SEQ ID NO: 579 - protein, 7 aa, Canis sp.
RDSNRPT

SEQ ID NO: 580 - protein, 12 aa, Canis sp.
CQVWDNSNKAMF

SEQ ID NO: 581 - DNA, 355 bp, Canis sp.
  source 1..355: mol_type = other DNA
gaggtgcaattggtggagtctgggggagacctggtgaagcctggggggtccctgagactttcctgtgtggcctctggattcaccttcaatacctaccacatgaactgggtccgccaggctccagggaaggggcttcactgggtcgcattcattaacagcggtggaagtggcacaagctatgcagacactgtgaagggccgattcaccatctccagagactacgccaagaacacactctatcttcagatgaacagcctgagagccgaggacacggccgtgtattactgtgcgagtggatctatatggactcattttgactactggggccagggaaccctggtcaccgtctcctcag

SEQ ID NO: 582 - protein, 118 aa, Canis sp.
EVQLVESGGDLVKPGGSLRLSCVASGFTFNTYHMNWVRQAPGKGLHWVAFINSGGSGTSYADTVKGRFTISRDYAKNTLYLQMNSLRAEDTAVYYCASGSIWTHFDYWGQGTLVTVSS

SEQ ID NO: 583 - DNA, 322 bp, Canis sp.
  source 1..322: mol_type = other DNA
tcctatgtgctgacacagctgccatccgtgagtttgaccctgaggcagacggcccacatcacctgtgggggagacatcattggaagtaaaagtgtttactggtaccagcagaagctgggccaggcccctgtattgattatctatagagataacaacaggccgacagggatccctgagcgattctctggcgccagctcggggaacacggccaccctgaccatcagcggggccctggccgaggacgaggctgactattactgccaggtttgggacagcagtactcaggctatgttcggcggaggcacccatctgaccgtcctcg

SEQ ID NO: 584 - protein, 107 aa, Canis sp.
SYVLTQLPSVSLTLRQTAHITCGGDIIGSKSVYWYQQKLGQAPVLIIYRDNNRPTGIPERFSGASSGNTATLTISGALAEDEADYYCQVWDSSTQAMFGGGTHLTVL

SEQ ID NO: 585 - protein, 7 aa, Canis sp.
GFTFNTY

SEQ ID NO: 586 - protein, 6 aa, Canis sp.
NSGGSG

SEQ ID NO: 587 - protein, 13 aa, Canis sp.
CASGSIWTHFDYW

SEQ ID NO: 588 - protein, 11 aa, Canis sp.
GGDIIGSKSVY

SEQ ID NO: 589 - protein, 7 aa, Canis sp.
RDNNRPT

SEQ ID NO: 590 - protein, 12 aa, Canis sp.
CQVWDSSTQAMF

SEQ ID NO: 591 - DNA, 355 bp, Canis sp.
  source 1..355: mol_type = other DNA
gaggtgcagctggtggagtctgggggagacctggtgaggcctggggggtccctgagactttcctgtgtggcctctggattcaccttcagtagctaccacatgaactgggtccgccaggctccagggaaggggcttcagtgggtcgcattcattaacagtggtggaagtagcacaacctatgcagacgttgtgaggggccgattcaccatctccagagactacgccaaaaacacgctgtatcttcagatgaacagcctgagagccgaggacacggccgtgtattactgtgcgagtggatctatatggactcattttgactactggggccagggaaccctggtcaccgtctcctcag

SEQ ID NO: 592 - protein, 118 aa, Canis sp.
EVQLVESGGDLVRPGGSLRLSCVASGFTFSSYHMNWVRQAPGKGLQWVAFINSGGSSTTYADVVRGRFTISRDYAKNTLYLQMNSLRAEDTAVYYCASGSIWTHFDYWGQGTLVTVSS

SEQ ID NO: 593 - DNA, 322 bp, Canis sp.
  source 1..322: mol_type = other DNA
tcctttgtgctgacacagctgccatccgtaagtgtgaccctgaggcagacggcccgcatcacctgtgggggagacagcattggaactaagagtgtttactggtaccagcagaagctgggccaggcccctgtattgattatctatagagatagcaacaggccgacagggatccctgagcgattctctggcgccagctcggggaacacggccaccctgaccatcagcggggccctggccgaggacgaggctgactattactgccaggtgtgggacagcagtaataaggctatgttcggcggaggcacccatctgaccgtcctcg

SEQ ID NO: 594 - protein, 107 aa, Canis sp.
SFVLTQLPSVSVTLRQTARITCGGDSIGTKSVYWYQQKLGQAPVLIIYRDSNRPTGIPERFSGASSGNTATLTISGALAEDEADYYCQVWDSSNKAMFGGGTHLTVL

SEQ ID NO: 595 - protein, 7 aa, Canis sp.
GFTFSSY

SEQ ID NO: 596 - protein, 6 aa, Canis sp.
NSGGSS

SEQ ID NO: 597 - protein, 13 aa, Canis sp.
CASGSIWTHFDYW

SEQ ID NO: 598 - protein, 11 aa, Canis sp.
GGDSIGTKSVY

SEQ ID NO: 599 - protein, 7 aa, Canis sp.
RDSNRPT

SEQ ID NO: 600 - protein, 12 aa, Canis sp.
CQVWDSSNKAMF

SEQ ID NO: 601 - DNA, 355 bp, Canis sp.
  source 1..355: mol_type = other DNA
gaggtgcagctggtggagtctgggggagacctggtgaggcctggggggtccctgagactttcctgtgtggcctctggattcaccttcagtagttaccacatgagctgggtccgccaggctcctgggaaggggcttcagtgggtcgcattcattaacagtggtggaagtagtacaacctatgcagacgttgtgaggggccgattcaccatctccagagactacgccaaaaacacgctgtatcttcagatgaacagcctgagagccgaggacacggccgtgtattattgtgcgagtggatctatatggactcattttgactactggggccagggaaccctggtcaccgtctcctcag

SEQ ID NO: 602 - protein, 118 aa, Canis sp.
EVQLVESGGDLVRPGGSLRLSCVASGFTFSSYHMSWVRQAPGKGLQWVAFINSGGSSTTYADVVRGRFTISRDYAKNTLYLQMNSLRAEDTAVYYCASGSIWTHFDYWGQGTLVTVSS

SEQ ID NO: 603 - DNA, 322 bp, Canis sp.
  source 1..322: mol_type = other DNA
tcctttgtgctgacacagctgccatccgtgagtgtgaccctgaggcagacggcccgcatcacctgtgggggagacagcattggaagtaaaagtgtttactggtaccagcagaggctgggccaggcccctgtattgattatctatagagatagcaacaggccgacagggatccctgagcgattctctggcgccagctcggggaacacggccaccctgaccatcagcggggccctggccgaggacgaggctgactattactgccaggtgtgggacaacagtaataaggctatgttcggcggaggcacccatctgaccgtcctcg

SEQ ID NO: 604 - protein, 107 aa, Canis sp.
SFVLTQLPSVSVTLRQTARITCGGDSIGSKSVYWYQQRLGQAPVLIIYRDSNRPTGIPERFSGASSGNTATLTISGALAEDEADYYCQVWDNSNKAMFGGGTHLTVL

SEQ ID NO: 605 - protein, 7 aa, Canis sp.
GFTFSSY

SEQ ID NO: 606 - protein, 6 aa, Canis sp.
NSGGSS

SEQ ID NO: 607 - protein, 13 aa, Canis sp.
CASGSIWTHFDYW

SEQ ID NO: 608 - protein, 11 aa, Canis sp.
GGDSIGSKSVY

SEQ ID NO: 609 - protein, 7 aa, Canis sp.
RDSNRPT

SEQ ID NO: 610 - protein, 12 aa, Canis sp.
CQVWDNSNKAMF

SEQ ID NO: 611 - DNA, 355 bp, Canis sp.
  source 1..355: mol_type = other DNA
gaggtgcagctggtggagtctgggggagacctggtgaggcctggggggtccctgagactttcctgtgtggcctctggattcaccttcagtaactaccacatgagctgggtccgccaggctccagggaaggggcttcagtgggtcgcattcattaacagtggtggaagtagcacaagctatgcagacgctgtgaggggccgattcaccatctccagagactacgccaagaacacgctgtatcttcagatgaacagcctgagagccgaggacacggccgtctattactgtgcgagtggatctatatggactcattttgactattggggccagggaaccctggtcaccgtctcctcag

SEQ ID NO: 612 - protein, 118 aa, Canis sp.
EVQLVESGGDLVRPGGSLRLSCVASGFTFSNYHMSWVRQAPGKGLQWVAFINSGGSSTSYADAVRGRFTISRDYAKNTLYLQMNSLRAEDTAVYYCASGSIWTHFDYWGQGTLVTVSS

SEQ ID NO: 613 - DNA, 322 bp, Canis sp.
  source 1..322: mol_type = other DNA
tcctttgtgctgacacagctgccatccgtgagtgtgaccctgaggcagacggcccgcatcacctgtgggggagacatcattggaagtaaaagtgtttactggttccagcagaagctgggccaggcccctatactgattatctatagagatgacaacaggccgacagggatccctgagcgattctctggcgccagctcggggaacacggccaccctgatcatcagcggggccctggccgaggacgaggctcactattactgccaggtgtgggacagcagttctaaggccatgttcggcggaggcacccatctgaccgtcctcg

SEQ ID NO: 614 - protein, 107 aa, Canis sp.
SFVLTQLPSVSVTLRQTARITCGGDIIGSKSVYWFQQKLGQAPILIIYRDDNRPTGIPERFSGASSGNTATLIISGALAEDEAHYYCQVWDSSSKAMFGGGTHLTVL

SEQ ID NO: 615 - protein, 7 aa, Canis sp.
GFTFSNY

SEQ ID NO: 616 - protein, 6 aa, Canis sp.
NSGGSS

SEQ ID NO: 617 - protein, 13 aa, Canis sp.
CASGSIWTHFDYW

SEQ ID NO: 618 - protein, 11 aa, Canis sp.
GGDIIGSKSVY

SEQ ID NO: 619 - protein, 7 aa, Canis sp.
RDDNRPT

SEQ ID NO: 620 - protein, 12 aa, Canis sp.
CQVWDSSSKAMF

SEQ ID NO: 621 - DNA, 355 bp, Canis sp.
  source 1..355: mol_type = other DNA
gaggtacaactggtggagtctgggggagacctggtgaggcctggggggtccctgagactttcctgtgtggcctctggattcaccttcaataattaccacatgagctgggtccgccaggctcctgggaaggggcttcagtgggtcgcatacattaacagtggtggaagtagcacaagttatgcagacgctgtgaagggccgattcaccatctccagagacaacgccaagaacacgctgtatcttcagatgaacagcctgagagccgaggacacggccttatattattgtgcgagtggatctatatggactcattttgactactggggccagggaaccctggtcaccgtctcctcag

SEQ ID NO: 622 - protein, 118 aa, Canis sp.
EVQLVESGGDLVRPGGSLRLSCVASGFTFNNYHMSWVRQAPGKGLQWVAYINSGGSSTSYADAVKGRFTISRDNAKNTLYLQMNSLRAEDTALYYCASGSIWTHFDYWGQGTLVTVSS

SEQ ID NO: 623 - DNA, 322 bp, Canis sp.
  source 1..322: mol_type = other DNA
tcctttgtgctgacacagctgccatccgtgagtgtgaccctgaggcagacggcccgcatcacctgtgggggagacatcattggaagtaaaaatgtttactggtaccagcagaggctgggccaggcccctgtattgattatctatagagatagtaacaggccgacagggatccctgagcgattctctggcgccagctcggggaacacggccaccctgaccatcagcggggccctggccgaggacgagggtgactattactgccaggtgtgggacaacagtaataaggctatattcggcggaggcacccatctgaccgtcctcg

SEQ ID NO: 624 - protein, 107 aa, Canis sp.
SFVLTQLPSVSVTLRQTARITCGGDIIGSKNVYWYQQRLGQAPVLIIYRDSNRPTGIPERFSGASSGNTATLTISGALAEDEGDYYCQVWDNSNKAIFGGGTHLTVL

SEQ ID NO: 625 - protein, 7 aa, Canis sp.
GFTFNNY

SEQ ID NO: 626 - protein, 6 aa, Canis sp.
NSGGSS

SEQ ID NO: 627 - protein, 13 aa, Canis sp.
CASGSIWTHFDYW

SEQ ID NO: 628 - protein, 11 aa, Canis sp.
GGDIIGSKNVY

SEQ ID NO: 629 - protein, 7 aa, Canis sp.
RDSNRPT

SEQ ID NO: 630 - protein, 12 aa, Canis sp.
CQVWDNSNKAIF

SEQ ID NO: 631 - DNA, 355 bp, Canis sp.
  source 1..355: mol_type = other DNA
gaggtacagctggtggagtctgggggagacctggtgaggcctggggggtccctgagactctcctgtgtggcctctggattcaccttcagtaacctccacatgaattgggtccgccaggctccagggaaggggcttcagtgggtcgcattcattaacagtggtggaagtggcacaacctatgcagacgttgtgaggggccgattcaccatctccagggactacgccaaaaacacactgaatcttcagatgactagcctgagagccgaggacacggccgtatattattgtgcgactggatctatatggactcattttgactattggggccagggaaccctggtcaccgtctcctcag

SEQ ID NO: 632 - protein, 118 aa, Canis sp.
EVQLVESGGDLVRPGGSLRLSCVASGFTFSNLHMNWVRQAPGKGLQWVAFINSGGSGTTYADVVRGRFTISRDYAKNTLNLQMTSLRAEDTAVYYCATGSIWTHFDYWGQGTLVTVSS

SEQ ID NO: 633 - DNA, 322 bp, Canis sp.
  source 1..322: mol_type = other DNA
tcctttgtgttgacacagctgccatccgtgagtgtgaccctgaggcagacggcccgcatcacctgtgggggagacagtattggaagtaaaagtgtttactggtaccagcagaagttgggccaggcccctgtattgattatctatagagatagcaacaggccgacagggatccctgagcgaatctctggcgccagctcggggaacacggccaccctgaccatcagcggggccctggccgaggacgaggctgactattactgccaggtgtgggacagcagtaataaggctatgttcggcggaggcacccatctgaccgtcctcg

SEQ ID NO: 634 - protein, 107 aa, Canis sp.; identical to SEQ ID NO: 544

SEQ ID NO: 635 - protein, 7 aa, Canis sp.
GFTFSNL

SEQ ID NO: 636 - protein, 6 aa, Canis sp.
NSGGSG

SEQ ID NO: 637 - protein, 13 aa, Canis sp.
CATGSIWTHFDYW

SEQ ID NO: 638 - protein, 11 aa, Canis sp.
GGDSIGSKSVY

SEQ ID NO: 639 - protein, 7 aa, Canis sp.
RDSNRPT

SEQ ID NO: 640 - protein, 12 aa, Canis sp.
CQVWDSSNKAMF

SEQ ID NO: 641 - DNA, 355 bp, Canis sp.
  source 1..355: mol_type = other DNA
gaggtgcagctggtggagtctgggggagacctggtgaggcctggggggtccctgagactttcctgtgtggcctctggattcaccttcagtaactaccacatgagctgggtccgccaggctccagggaaggggcttcagtgggtcgcattcattaacagtggtggaactagtacaacctatgcagacgctgtgaggggccgattcaccacctccagagactacgccaagaacacgctgtatcttcagatgaacagcctgagagccgaggacacggccgtctattactgtacgagtggatctatatggactcattttgactattggggccagggaaccctggtcaccgtctcctcag

SEQ ID NO: 642 - protein, 118 aa, Canis sp.
EVQLVESGGDLVRPGGSLRLSCVASGFTFSNYHMSWVRQAPGKGLQWVAFINSGGTSTTYADAVRGRFTTSRDYAKNTLYLQMNSLRAEDTAVYYCTSGSIWTHFDYWGQGTLVTVSS

SEQ ID NO: 643 - DNA, 322 bp, Canis sp.; identical to SEQ ID NO: 613
  source 1..322: mol_type = other DNA

SEQ ID NO: 644 - protein, 107 aa, Canis sp.; identical to SEQ ID NO: 614

SEQ ID NO: 645 - protein, 7 aa, Canis sp.
GFTFSNY

SEQ ID NO: 646 - protein, 6 aa, Canis sp.
NSGGTS

SEQ ID NO: 647 - protein, 13 aa, Canis sp.
CTSGSIWTHFDYW

SEQ ID NO: 648 - protein, 11 aa, Canis sp.
GGDIIGSKSVY

SEQ ID NO: 649 - protein, 7 aa, Canis sp.
RDDNRPT

SEQ ID NO: 650 - protein, 12 aa, Canis sp.
CQVWDSSSKAMF

SEQ ID NO: 651 - DNA, 355 bp, Canis sp.
  source 1..355: mol_type = other DNA
gaggtgcaattggtggagtctggtggagacctggtgaagcctggggggtccctgagactttcctgtgtggcctctggattcaccttcaatacctaccacatgaactgggtccgccaggctccagggaaggggcttcactgggtcgcattcattaacagcggtggaagtggcacaagctatgaagacactgtgcagggccgattcaccatctccagagactacgccaagaacacactctatcttcagatgaacagcctgagagccgaggacacggccgtatattactgtgcgtgtggatctatatggactcattttgactactggggccagggaaccctggtcaccgtctcctcag

SEQ ID NO: 652 - protein, 118 aa, Canis sp.
EVQLVESGGDLVKPGGSLRLSCVASGFTFNTYHMNWVRQAPGKGLHWVAFINSGGSGTSYEDTVQGRFTISRDYAKNTLYLQMNSLRAEDTAVYYCACGSIWTHFDYWGQGTLVTVSS

SEQ ID NO: 653 - DNA, 322 bp, Canis sp.; identical to SEQ ID NO: 563
  source 1..322: mol_type = other DNA

SEQ ID NO: 654 - protein, 107 aa, Canis sp.; identical to SEQ ID NO: 564

SEQ ID NO: 655 - protein, 7 aa, Canis sp.
GFTFNTY

SEQ ID NO: 656 - protein, 6 aa, Canis sp.
NSGGSG

SEQ ID NO: 657 - protein, 13 aa, Canis sp.
CACGSIWTHFDYW

SEQ ID NO: 658 - protein, 11 aa, Canis sp.
GGDIIGSKSVY

SEQ ID NO: 659 - protein, 7 aa, Canis sp.
RDNNRPT

SEQ ID NO: 660 - protein, 12 aa, Canis sp.
CQVWDSRTQALF

SEQ ID NO: 661 - DNA, 352 bp, Canis sp.
  source 1..352: mol_type = other DNA
gaggtgcagctggtggagtctgggggagacctggtgaagcctggggggtccctgagactttcctgtgtggcctctggattcaccttcagtagctaccacatgagctgggtccgccaggctccagggaaggggcttcagtgggtcgcatacattaacagtggtggaagtagcacaagctatgcagacgctgtgaagggccgattcaccatctccagagacaacgccaagaacacgctgtatcttcagatgaacagcctgagagccgaggacacggccgtgtattactgtgcgggctttatatggactcattttgactactggggccagggaaccctggtcaccgtctcctcag

SEQ ID NO: 662 - protein, 117 aa, Canis sp.
EVQLVESGGDLVKPGGSLRLSCVASGFTFSSYHMSWVRQAPGKGLQWVAYINSGGSSTSYADAVKGRFTISRDNAKNTLYLQMNSLRAEDTAVYYCAGFIWTHFDYWGQGTLVTVSS

SEQ ID NO: 663 - DNA, 334 bp, Canis sp.
  source 1..334: mol_type = other DNA
gatattgtcatgacacagacgccaccgtccctgtctgtcagccctagagagacggcctccatctcctgcaaggccagtcagagcctcctgcacagtgatggaaacacctatttggattggtacctgcaaaagccaggccagtctccacagcttctgatctacttggtttccaaccgcttcactggcgtgtcagacaggttcagtggcagcgggtcagggacagatttcaccctgagaatcagcagagtggaggctaacgatactggagtttattactgcgggcaaggtacacagctgtggacgttcggagcaggaaccaaggtggagctcaaac

SEQ ID NO: 664 - protein, 111 aa, Canis sp.
DIVMTQTPPSLSVSPRETASISCKASQSLLHSDGNTYLDWYLQKPGQSPQLLIYLVSNRFTGVSDRFSGSGSGTDFTLRISRVEANDTGVYYCGQGTQLWTFGAGTKVELK

SEQ ID NO: 665 - protein, 7 aa, Canis sp.
GFTFSSY

SEQ ID NO: 666 - protein, 6 aa, Canis sp.
NSGGSS

SEQ ID NO: 667 - protein, 12 aa, Canis sp.
CAGFIWTHFDYW

SEQ ID NO: 668 - protein, 16 aa, Canis sp.
KASQSLLHSDGNTYLD

SEQ ID NO: 669 - protein, 7 aa, Canis sp.
LVSNRFT

SEQ ID NO: 670 - protein, 10 aa, Canis sp.
CGQGTQLWTF

SEQ ID NO: 671 - DNA, 355 bp, Canis sp.
  source 1..355: mol_type = other DNA
gcggtgcagctggtggagtctgggggagacctggtgaggcctggggggtccctgagactttcctgtgtggcctctggattcaccttcagtagttaccacatgaactgggtccgccaggctcctgggaaggggcttcagtgggtcgcattcattaacagtggaggaagtagtacaacctatgcagacgttgtcaggggccgattcaccatctccagagactacgccaaaaacacgctatttcttcagatgaacagcctgagagccgaggacacggccgtgtattattgtgcgattgtatctatatggacacattttgactactggggccagggaaccctggtcaccgtctcctcag

SEQ ID NO: 672 - protein, 118 aa, Canis sp.
AVQLVESGGDLVRPGGSLRLSCVASGFTFSSYHMNWVRQAPGKGLQWVAFINSGGSSTTYADVVRGRFTISRDYAKNTLFLQMNSLRAEDTAVYYCAIVSIWTHFDYWGQGTLVTVSS

SEQ ID NO: 673 - DNA, 322 bp, Canis sp.; identical to SEQ ID NO: 553
  source 1..322: mol_type = other DNA

SEQ ID NO: 674 - protein, 107 aa, Canis sp.; identical to SEQ ID NO: 554

SEQ ID NO: 675 - protein, 7 aa, Canis sp.
GFTFSSY

SEQ ID NO: 676 - protein, 6 aa, Canis sp.
NSGGSS

SEQ ID NO: 677 - protein, 13 aa, Canis sp.
CAIVSIWTHFDYW

SEQ ID NO: 678 - protein, 11 aa, Canis sp.
GGDIIGSKNVY

SEQ ID NO: 679 - protein, 7 aa, Canis sp.
RDTNRPT

SEQ ID NO: 680 - protein, 12 aa, Canis sp.
CQVWDNSNKAMF

SEQ ID NO: 681 - DNA, 355 bp, Canis sp.
  source 1..355: mol_type = other DNA
gaggtgcagctggtggagtctgggggagacctggtgaggccgggggggtccctgagactttcctgtgtggcctctggattcaccttcactaactaccacatgagctgggtccgccaggctccagggaaggggcttcagtgggtcgcattcattaacagtggtggaagtagcacaacctatacagacgttgtgaggggccgattcaccatctccagagactacgccaaaaacacgctgtatcttcagatgaacagcctgacagccgaggacacggccgtgtattactgtgcgagtggagctatatggactcattttgaccactggggccagggaaccctggtcaccgtctcctcag

SEQ ID NO: 682 - protein, 118 aa, Canis sp.
EVQLVESGGDLVRPGGSLRLSCVASGFTFTNYHMSWVRQAPGKGLQWVAFINSGGSSTTYTDVVRGRFTISRDYAKNTLYLQMNSLTAEDTAVYYCASGAIWTHFDHWGQGTLVTVSS

SEQ ID NO: 683 - DNA, 322 bp, Canis sp.
  source 1..322: mol_type = other DNA
tcctttgtactgacacagctgccatccgtgagtgtgaccctgaggcagacggcccgcatcacctgtgggggagacagcgttggaagtaaaagtgtttactggtaccagcagaagctgggccaggcccctgtactgattatctatagagatagcaacaggccgacagggatccctgagcgattctctggcgccagctcggggaacacggccaccctgaccatcagcggggccctggccgaggacgaggctgactattactgccaggtgtgggacaacagtaataaggctatattcggcggaggcacccatctgaccgtcctcg

SEQ ID NO: 684 - protein, 107 aa, Canis sp.
SFVLTQLPSVSVTLRQTARITCGGDSVGSKSVYWYQQKLGQAPVLIIYRDSNRPTGIPERFSGASSGNTATLTISGALAEDEADYYCQVWDNSNKAIFGGGTHLTVL

SEQ ID NO: 685 - protein, 7 aa, Canis sp.
GFTFTNY

SEQ ID NO: 686 - protein, 6 aa, Canis sp.
NSGGSS

SEQ ID NO: 687 - protein, 13 aa, Canis sp.
CASGAIWTHFDHW

SEQ ID NO: 688 - protein, 11 aa, Canis sp.
GGDSVGSKSVY

SEQ ID NO: 689 - protein, 7 aa, Canis sp.
RDSNRPT

SEQ ID NO: 690 - protein, 12 aa, Canis sp.
CQVWDNSNKAIF

SEQ ID NO: 691 - DNA, 355 bp, Canis sp.
  source 1..355: mol_type = other DNA
gaggtgcagctggtggagtctgggggagacctggtgaggccgggggggtccctgagactttcctgtgtggcctctggattcaccttcactaactaccacatgagctgggtccgccaggctccagggaaggggcttcagtgggtcgcattcattaacagtggtggaagtagcacaacctatacagacgttgtgaggggccgattcaccatctccagagactacgccaaaaacacgctgtatcttcagatgaacagcctgacagccgaggacacggccgtgtattactgtgcgagtggatctatatggactcattttgaccactggggccagggaaccctggtcaccgtctcctcag

SEQ ID NO: 692 - protein, 118 aa, Canis sp.
EVQLVESGGDLVRPGGSLRLSCVASGFTFTNYHMSWVRQAPGKGLQWVAFINSGGSSTTYTDVVRGRFTISRDYAKNTLYLQMNSLTAEDTAVYYCASGSIWTHFDHWGQGTLVTVSS

SEQ ID NO: 693 - DNA, 322 bp, Canis sp.; identical to SEQ ID NO: 683
  source 1..322: mol_type = other DNA

SEQ ID NO: 694 - protein, 107 aa, Canis sp.; identical to SEQ ID NO: 684

SEQ ID NO: 695 - protein, 7 aa, Canis sp.
GFTFTNY

SEQ ID NO: 696 - protein, 6 aa, Canis sp.
NSGGSS

SEQ ID NO: 697 - protein, 13 aa, Canis sp.
CASGSIWTHFDHW

SEQ ID NO: 698 - protein, 11 aa, Canis sp.
GGDSVGSKSVY

SEQ ID NO: 699 - protein, 7 aa, Canis sp.
RDSNRPT

SEQ ID NO: 700 - protein, 12 aa, Canis sp.
CQVWDNSNKAIF

SEQ ID NO: 701 - DNA, 355 bp, Canis sp.
  source 1..355: mol_type = other DNA
gaggtgcagctggtggagtctgggggagacctggtgaggcctggggggtccctgagactttcctgtgtggcctctggattcaccttcagtagctaccacatgagctgggtccgccaggctccagggaaggggcttcagtgggtcgcattcattaacagtggtggaagtagcacaacctatgcagacgttgtgaggggccgattcaccatctccagagactacgccaaaaacacgctgtatcttcagatgaacagcctgagagccgaggacacggccgtgtattactgtgcgagtggatctatattgactcattttgactactggggccagggaaccctggtcaccgtctcctcag

SEQ ID NO: 702 - protein, 118 aa, Canis sp.
EVQLVESGGDLVRPGGSLRLSCVASGFTFSSYHMSWVRQAPGKGLQWVAFINSGGSSTTYADVVRGRFTISRDYAKNTLYLQMNSLRAEDTAVYYCASGSILTHFDYWGQGTLVTVSS

SEQ ID NO: 703 - DNA, 322 bp, Canis sp.
  source 1..322: mol_type = other DNA
tcctttgtgctgacacagctgccatccgtgagtgtgaccctgaggcagacggcccgcatcacctgtgggggagacagcgttggaagtaaaagtgtttactggtaccagcagaagctgggccaggcccctgtactgattatctatagagatagcaacaggccgacagggatccctgagcgattctctggcgccagctcggggaacacggccaccctgaccatcagcggggccctggccgaggacgaggctgactattactgccaggtgtgggacaacagtaataaggctatgttcggcggaggcacccatctgaccgtcctcg

SEQ ID NO: 704 - protein, 107 aa, Canis sp.
SFVLTQLPSVSVTLRQTARITCGGDSVGSKSVYWYQQKLGQAPVLIIYRDSNRPTGIPERFSGASSGNTATLTISGALAEDEADYYCQVWDNSNKAMFGGGTHLTVL

SEQ ID NO: 705 - protein, 7 aa, Canis sp.
GFTFSSY

SEQ ID NO: 706 - protein, 6 aa, Canis sp.
NSGGSS

SEQ ID NO: 707 - protein, 13 aa, Canis sp.
CASGSILTHFDYW

SEQ ID NO: 708 - protein, 11 aa, Canis sp.
GGDSVGSKSVY

SEQ ID NO: 709 - protein, 7 aa, Canis sp.
RDSNRPT

SEQ ID NO: 710 - protein, 12 aa, Canis sp.
CQVWDNSNKAMF

SEQ ID NO: 711 - DNA, 355 bp, Canis sp.
  source 1..355: mol_type = other DNA
gaggtgcagctggtggagtctgggggagacctggtgaggccgggggggtccctgagactttcctgtgtggcctctggattcaccttcagtagctaccacatgaactgggtccgccaggctccagggaaggggcttcagtgggtcgcattcattaatagtggagcaagtagcgcaacctatgcagacgttgtgaggggccgattcaccatctccagagactacgccaaaaacacgctgtatcttcagatgaacagcctgagagccgaggacacggccgtgtattattgtgcgagtggatctatttggactcattttgacttctggggccagggaaccctggtcaccgtctcctcag

SEQ ID NO: 712 - protein, 118 aa, Canis sp.
EVQLVESGGDLVRPGGSLRLSCVASGFTFSSYHMNWVRQAPGKGLQWVAFINSGASSATYADVVRGRFTISRDYAKNTLYLQMNSLRAEDTAVYYCASGSIWTHFDFWGQGTLVTVSS

SEQ ID NO: 713 - DNA, 322 bp, Canis sp.; identical to SEQ ID NO: 703
  source 1..322: mol_type = other DNA

SEQ ID NO: 714 - protein, 107 aa, Canis sp.; identical to SEQ ID NO: 704

SEQ ID NO: 715 - protein, 7 aa, Canis sp.
GFTFSSY

SEQ ID NO: 716 - protein, 6 aa, Canis sp.
NSGASS

SEQ ID NO: 717 - protein, 13 aa, Canis sp.
CASGSIWTHFDFW

SEQ ID NO: 718 - protein, 11 aa, Canis sp.
GGDSVGSKSVY

SEQ ID NO: 719 - protein, 7 aa, Canis sp.
RDSNRPT

SEQ ID NO: 720 - protein, 12 aa, Canis sp.
CQVWDNSNKAMF

SEQ ID NO: 721 - protein, 335 aa, Canis sp.
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 722 - protein, 335 aa, Canis sp.
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLDPEDPEVQISWFVDGKQMQTAKTQPREEQFNGTYRVVSVLPIGHQDWLKGKQFTCKVNHIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 723 - protein, 335 aa, Canis sp.
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 724 - protein, 335 aa, Canis sp.
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLPSSIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 725 - protein, 335 aa, Canis sp.
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 726 - protein, 335 aa, Canis sp.
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 727 - protein, 335 aa, Canis sp.
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSSIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 728 - protein, 335 aa, Canis sp.
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 729 - protein, 335 aa, Canis sp.
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPGMLGGPSVFIFPPKPKDTLLIARTPEVTCVVVDLDPENPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 730 - protein, 335 aa, Canis sp.
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPENPEVQISWFVDGKQMQTAKTQPREEQFNGTYRVVSVLPIGHQDWLKGKQFTCKVNNKALPSSIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 731 - protein, 476 aa, Canis sp.
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 732 - DNA, 894 bp, Canis sp.
  source 1..894: mol_type = other DNA
atgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaa

SEQ ID NO: 733 - protein, 475 aa, Canis sp.
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 734 - protein, 471 aa, Canis sp.
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSTSGSTVALACLVSGYFPEPVTVSWNSGSLTSGVHTFPSVLQSSGLYSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 735 - protein, 473 aa, Canis sp.
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSQSGSTVALACLVSGYIPEPVTVSWNSGSLTSGVHTFPSILQSSGLYSLSSMVTVPSSRWPSETFTCNVAHPATNTKVDKPVVKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQKSLSHSPGK

SEQ ID NO: 736 - protein, 471 aa, Canis sp.
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASSTAPSVFPLAPSCGSTSGSTVALACLVSGYFPEPVTVSWNSGSLTSGVHTFPSVLKSSGLYSLSSMVTVPSSRLPSETFTCNVVHPATNTKVDKPVPKESTCKCISPCPVPESLGGPSVFIFPPKPKDILRITRTPEVTCVVLDLGREDPEVQISWFVDGKEVHTAKTQPREQQFNSTYRVVSVLPIEHQDWLTGKEFKCRVNHIGLPSPIERTISKARGQAHQPGVYVLPPSPKELSSSDTVTLTCLIKDFFPPEIDVEWQSNGQPEPESKYHTTAPQLDEDGSYFLYSKLSVDKSRWQQGDPFTCAVMHEALQNHYTDLSLSHSPGK

SEQ ID NO: 737 - DNA, 355 bp, Canis sp.
  source 1..355: mol_type = other DNA
gaggtgcagttggtggagtctgggggagaccttgtgaaacctgaggggtccctgagactctcctgtgtggtctctggcttcaccttcagtggcttcgacatgagctgggtccgccaggctccagggaaggggctgcagtgggtcgcatacattagtactgatggaaggagcacaggttacacagacgctgtgaagggccgattcaccatctccagagacaatgtcaagaacacgctgtatctgcagatgaacagcctgagaactgaggacacagccgtgtattactgtgcgaaggaaactcgtcattggggctttgactactggggccagggaaccctagtcatcgtctcctcag

SEQ ID NO: 738 - protein, 118 aa, Canis sp.
EVQLVESGGDLVKPEGSLRLSCVVSGFTFSGFDMSWVRQAPGKGLQWVAYISTDGRSTGYTDAVKGRFTISRDNVKNTLYLQMNSLRTEDTAVYYCAKETRHWGFDYWGQGTLVIVSS

SEQ ID NO: 739 - DNA, 319 bp, Canis sp.
  source 1..319: mol_type = other DNA
tcctatgtgttgacacagctgtcatccgtgagtgtgagcctgaggcagacggcccacatcacctgtgggggagacagcattggacgtaaaaatgtttactggcaccagcagaagctgggccgggcccctatactgattatctatgatgatagcagcaggccgtcagggatccctgagcgattctccggcgccaactcggggaacacggccaccctgaccatcagcggggccctggccgaggatgaggctgcctattactgccaggtgtgggacagcagtactagtgtgttcggcggaggcacccatctgaccgtcctcg

SEQ ID NO: 740 - protein, 106 aa, Canis sp.
SYVLTQLSSVSVSLRQTAHITCGGDSIGRKNVYWHQQKLGRAPILIIYDDSSRPSGIPERFSGANSGNTATLTISGALAEDEAAYYCQVWDSSTSVFGGGTHLTVL

SEQ ID NO: 741 - protein, 7 aa, Canis sp.
GFTFSGF

SEQ ID NO: 742 - protein, 6 aa, Canis sp.
STDGRS

SEQ ID NO: 743 - protein, 13 aa, Canis sp.
CAKETRHWGFDYW

SEQ ID NO: 744 - protein, 11 aa, Canis sp.
GGDSIGRKNVY

SEQ ID NO: 745 - protein, 7 aa, Canis sp.
DDSSRPS

SEQ ID NO: 746 - protein, 11 aa, Canis sp.
CQVWDSSTSVF

SEQ ID NO: 747 - DNA, 361 bp, Canis sp.
  source 1..361: mol_type = other DNA
gagttgcacctagtggagtctgggggagacctggtgaagcctgcggggtccctgagactgtcctgtgtggcctctggattcaccttcagtagcttcagaatgagttgggtccgccagtctcctgggaagggactgcaatttgtcgcaggtatcaacagcgatggatataacacacagtacagagacgatgtgaagggtcgattcaccatctccagagacaacaccgaaaagacactatatctacagatgaacaacctgagagtcgaggacacggccatgtattactgtgtaaaggatcagggaagttggggaggaaattttgacttctggggccagggaaccctggtcaccgtctcctcag

SEQ ID NO: 748 - protein, 120 aa, Canis sp.
ELHLVESGGDLVKPAGSLRLSCVASGFTFSSFRMSWVRQSPGKGLQFVAGINSDGYNTQYRDDVKGRFTISRDNTEKTLYLQMNNLRVEDTAMYYCVKDQGSWGGNFDFWGQGTLVTVSS

SEQ ID NO: 749 - DNA, 319 bp, Canis sp.
  source 1..319: mol_type = other DNA
tcctatggcctgactcagccaccatccgtgaatgtgaccctgagggagacggcccacatcacctgtgggggagacagcattggaagtaaatatgttcaatggatccagcagattccaggccaggcccccgtagtgattatctataaagataggaacaggccgacagggatccctgagcgattctctggcgccaactcagggaacacggctaccctgaccatcagtggggccctggccgaagacgaggctgactattactgccaggtgggggacagtggtactagtgtgttcggcggaggcacccatctgaccgtcctcg

SEQ ID NO: 750 - protein, 106 aa, Canis sp.
SYGLTQPPSVNVTLRETAHITCGGDSIGSKYVQWIQQIPGQAPVVIIYKDRNRPTGIPERFSGANSGNTATLTISGALAEDEADYYCQVGDSGTSVFGGGTHLTVL

SEQ ID NO: 751 - protein, 7 aa, Canis sp.
GFTFSSF

SEQ ID NO: 752 - protein, 6 aa, Canis sp.
NSDGYN

SEQ ID NO: 753 - protein, 15 aa, Canis sp.
CVKDQGSWGGNFDFW

SEQ ID NO: 754 - protein, 11 aa, Canis sp.
GGDSIGSKYVQ

SEQ ID NO: 755 - protein, 7 aa, Canis sp.
KDRNRPT

SEQ ID NO: 756 - protein, 11 aa, Canis sp.
CQVGDSGTSVF

SEQ ID NO: 757 - DNA, 361 bp, Canis sp.
  source 1..361: mol_type = other DNA
gagttgcagctggtggagtctgggggagacctggtgaagcctgcggggtccctgagactgtcctgtgtggcctctggattcaccttcagtaacttcagaatgagttgggtccgccaggctcctgggaagggactgcaatttgtcgcaggtatcaacagcgatggatatagcacacagtacagagacgatgtgaagggtcgattcaccatctccagagacaacaccgaaaagacactatatctacagatgaacaacctgagagtcgaggacacggccatgtattactgtgtaaaggatcagggaagttggggaggaaattttgactactggggccagggaaccctggtcaccgtctcctcag

SEQ ID NO: 758 - protein, 120 aa, Canis sp.
ELQLVESGGDLVKPAGSLRLSCVASGFTFSNFRMSWVRQAPGKGLQFVAGINSDGYSTQYRDDVKGRFTISRDNTEKTLYLQMNNLRVEDTAMYYCVKDQGSWGGNFDYWGQGTLVTVSS

SEQ ID NO: 759 - DNA, 319 bp, Canis sp.; identical to SEQ ID NO: 749
  source 1..319: mol_type = other DNA

SEQ ID NO: 760 - protein, 106 aa, Canis sp.; identical to SEQ ID NO: 750

SEQ ID NO: 761 - protein, 7 aa, Canis sp.
GFTFSNF

SEQ ID NO: 762 - protein, 6 aa, Canis sp.
NSDGYS

SEQ ID NO: 763 - protein, 15 aa, Canis sp.
CVKDQGSWGGNFDYW

SEQ ID NO: 764 - protein, 11 aa, Canis sp.
GGDSIGSKYVQ

SEQ ID NO: 765 - protein, 7 aa, Canis sp.
KDRNRPT

SEQ ID NO: 766 - protein, 11 aa, Canis sp.
CQVGDSGTSVF

SEQ ID NO: 767 - DNA, 358 bp, Canis sp.
  source 1..358: mol_type = other DNA
gaggtgcagctggtggagtctgggggagacctgttgaagcctggggggtccctgagactctcctgtgtagcctctggattccccttcaggaactacgacatgacctgggtccgccaggctcctgggaaggggctgcagtgggtcgccgctattcactatgatggaagtaacacatactacactgacgctgtgaagggccgattcaccatttccagagacaacgccaggaacacagtgtttctgcagatgaacagcctgagagccgaggacacggctgtgtattactgtgcgaagaaatttaataattaccgtccttttgattactggggccagggaaccctggtcaccgtctcctcag

SEQ ID NO: 768 - protein, 119 aa, Canis sp.
EVQLVESGGDLLKPGGSLRLSCVASGFPFRNYDMTWVRQAPGKGLQWVAAIHYDGSNTYYTDAVKGRFTISRDNARNTVFLQMNSLRAEDTAVYYCAKKFNNYRPFDYWGQGTLVTVSS

SEQ ID NO: 769 - DNA, 337 bp, Canis sp.
  source 1..337: mol_type = other DNA
gatattgtcatgacacagacgccaccgtccctgtctgtcagccctagagagacgccctccatctcctgcaaggccagtcagagcctcctgcgcagtgatggaaacacctatttggaatggtacctgcaaaagcccggccagtctccacagcttctgatctacttggtttccaaccgcttcactggcgtgtcagacaggttcagtggcagcgggtcagggacagatttcaccctgagaatcagcagagtggaggctaacgatgctggagtttattactgcgggcaaggtacacagcttccgtggacgttcggagcaggaaccaaggtggagctcaaac

SEQ ID NO: 770 - protein, 112 aa, Canis sp.
DIVMTQTPPSLSVSPRETPSISCKASQSLLRSDGNTYLEWYLQKPGQSPQLLIYLVSNRFTGVSDRFSGSGSGTDFTLRISRVEANDAGVYYCGQGTQLPWTFGAGTKVELK

SEQ ID NO: 771 - protein, 7 aa, Canis sp.
GFPFRNY

SEQ ID NO: 772 - protein, 6 aa, Canis sp.
HYDGSN

SEQ ID NO: 773 - protein, 14 aa, Canis sp.
CAKKFNNYRPFDYW

SEQ ID NO: 774 - protein, 16 aa, Canis sp.
KASQSLLRSDGNTYLE

SEQ ID NO: 775 - protein, 7 aa, Canis sp.
LVSNRFT

SEQ ID NO: 776 - protein, 11 aa, Canis sp.
CGQGTQLPWTF

SEQ ID NO: 777 - DNA, 349 bp, Canis sp.
  source 1..349: mol_type = other DNA
gcggtgcagctggtggagtctgggggagacctggtgaggccgggggggtccctgagactctcctgtgtagcctctggattcaccttcagtaacttcgacatgtactgggtccgccaggctcctgggagggggctgcagtgggtcgcaactatttcctatgatggatttaacacatactccactgacactgtgcggggccgattcaccatctccagagacaacgccaggaacacagtgtatctgcagatgaacagtctcagagtcgaggacacggctgttttttactgtacggctaggaggtttggacttgggtactggggccagggaaccctggtcaccgtctcctcag

SEQ ID NO: 778 - protein, 116 aa, Canis sp.
AVQLVESGGDLVRPGGSLRLSCVASGFTFSNFDMYWVRQAPGRGLQWVATISYDGFNTYSTDTVRGRFTISRDNARNTVYLQMNSLRVEDTAVFYCTARRFGLGYWGQGTLVTVSS

SEQ ID NO: 779 - DNA, 337 bp, Canis sp.
  source 1..337: mol_type = other DNA
gatattttcatggcacagacgccaccgtccctgtctgtcagccctagagagacggcctccatctcctgtaaggccagtcagagcctcctgcacagtgatggaaacacctatttggattggtacctgcaaaagccaggccagtctccacagcttctgatctacttggtttccaaccgcttccctggcgtgtcagacaggttcagtggcagcgggtcagggacaggtttcaccctgagaatcagcagagtggaggctagtgatactggagtttattactgcgggcaaggtacacagcttccttataccttcagccagggaaccaagctggagataaaac

SEQ ID NO: 780 - protein, 112 aa, Canis sp.
DIFMAQTPPSLSVSPRETASISCKASQSLLHSDGNTYLDWYLQKPGQSPQLLIYLVSNRFPGVSDRFSGSGSGTGFTLRISRVEASDTGVYYCGQGTQLPYTFSQGTKLEIK

SEQ ID NO: 781 - protein, 7 aa, Canis sp.
GFTFSNF

SEQ ID NO: 782 - protein, 6 aa, Canis sp.
SYDGFN

SEQ ID NO: 783 - protein, 11 aa, Canis sp.
CTARRFGLGYW

SEQ ID NO: 784 - protein, 16 aa, Canis sp.
KASQSLLHSDGNTYLD

SEQ ID NO: 785 - protein, 7 aa, Canis sp.
LVSNRFP

SEQ ID NO: 786 - protein, 11 aa, Canis sp.
CGQGTQLPYTF

SEQ ID NO: 787 - DNA, 355 bp, Canis sp.
  source 1..355: mol_type = genomic DNA
gaggtgcgactgctggagtctgggggagaccttgtgaaacctgcggggtccctgagactctcctgtgcggtttctggcttcaccttcagtacctacgacatgagttgggtccgccaggctccagggaaggggctgcagtgggtcgcatacattacctatgatggaaggaccacaagttacacagacactgtgaagggccgattcaccgtctccagagacaatgccaagaacatgctgtatctgaagatgaacagcctgagaagtgaggacgcagccgtttattactgtgcgaaggcccatagggggtggttttttgaccactggggccagggaaccctggtcaccgtctcctcag

SEQ ID NO: 788 - protein, 118 aa, Canis sp.
EVRLLESGGDLVKPAGSLRLSCAVSGFTFSTYDMSWVRQAPGKGLQWVAYITYDGRTTSYTDTVKGRFTVSRDNAKNMLYLKMNSLRSEDAAVYYCAKAHRGWFFDHWGQGTLVTVSS

SEQ ID NO: 789 - protein, 319 aa, Canis sp.
TCCTATGTGCTGACACAGCTGCCATCCGTGAGTGTGACCCTGAGGCAGACGGCCCGCATCACCTGTGGGGGAGACAGTATTGGAAGTAAAAATGTTTACTGGTACCAACAGAAACTGGGCCAGGCCCCTGTATTGATTATCTATGATGATACCAGCAGGCCGTCAGGGATCCCTGAGCGATTCTCCGGCGCCAATTCGGGGACCACGGCCACCCTGACCATCAGCGGGGCCCTGGCCGAGGATGAGGCTGACTATTACTGCCAGGTGTGGGACAGCAGTACTGGTGTGTTCGGCGGAGGCACCCACCTGACCGTCCTCG

SEQ ID NO: 790 - protein, 106 aa, Canis sp.
SYVLTQLPSVSVTLRQTARITCGGDSIGSKNVYWYQQKLGQAPVLIIYDDTSRPSGIPERFSGANSGTTATLTISGALAEDEADYYCQVWDSSTGVFGGGTHLTVL

SEQ ID NO: 791 - protein, 7 aa, Canis sp.
GFTFSTY

SEQ ID NO: 792 - protein, 6 aa, Canis sp.
TYDGRT

SEQ ID NO: 793 - protein, 13 aa, Canis sp.
CAKAHRGWFFDHW

SEQ ID NO: 794 - protein, 11 aa, Canis sp.
GGDSIGSKNVY

SEQ ID NO: 795 - protein, 7 aa, Canis sp.
DDTSRPS

SEQ ID NO: 796 - protein, 11 aa, Canis sp. [sequence omitted]

SEQ ID NO: 797 - DNA, 355 bp, Canis sp.
  source 1..355: mol_type = other DNA
gaggtgcagctggtggagtctgggggagaccttgtgaaacctgaggggtccctgagactctcctgtgcggtctctggcttcaccttcagtagtaacgacatgagttgggtccgccaggctccagggaaggggctgcagtgggtcgcatacattacctatgatggaaggagcacaagttacacagacattgtgaagggccgattcaccgtctccagagacaatgccaagaacacgctgtatctgcagatgaatagcctgagaagtgaggacgcagccgtgtactactgtgcgaaggcccagagggggtggttttttgactactggggccagggaaccctggtcaccgtctcctcag

SEQ ID NO: 798 - protein, 118 aa, Canis sp.
EVQLVESGGDLVKPEGSLRLSCAVSGFTFSSNDMSWVRQAPGKGLQWVAYITYDGRSTSYTDIVKGRFTVSRDNAKNTLYLQMNSLRSEDAAVYYCAKAQRGWFFDYWGQGTLVTVSS

SEQ ID NO: 799 - DNA, 319 bp, Canis sp.
  source 1..319: mol_type = other DNA
tcctatgtgctgacacagctgccatccgtgagtgtgaccctgaggcagacggcccgcatcacctgtgggggagacagtattggaagtaaaaatgtttactggtaccaacagaaactgggccaggcccctgtactgattatctatgatgatgacagcaggccgtcagggatccctgagcgaatctccggcgccaattcggggaacacggccaccctgaccatcagcggggccctggccgaggatgaggctgactattactgccaggtgtgggacagcagtactggtgtgttcggcggaggcacccacctgaccgtcctcg

SEQ ID NO: 800 - protein, 106 aa, Canis sp.
SYVLTQLPSVSVTLRQTARITCGGDSIGSKNVYWYQQKLGQAPVLIIYDDDSRPSGIPERISGANSGNTATLTISGALAEDEADYYCQVWDSSTGVFGGGTHLTVL

SEQ ID NO: 801 - protein, 7 aa, Canis sp.
GFTFSSN

SEQ ID NO: 802 - protein, 6 aa, Canis sp.
TYDGRS

SEQ ID NO: 803 - protein, 13 aa, Canis sp.
CAKAQRGWFFDYW

SEQ ID NO: 804 - protein, 11 aa, Canis sp.
GGDSIGSKNVY

SEQ ID NO: 805 - protein, 7 aa, Canis sp.
DDDSRPS

SEQ ID NO: 806 - protein, 11 aa, Canis sp. [sequence omitted]

SEQ ID NO: 807 - protein, 356 aa, Canis sp.
NGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSHHHHHHENLYFQGQVPPQYPPIQSIRVQFTRCENEKGCIITSPSKDETMKVQDNSIIINCDGFYLISLKGYFSEELSLSLYYRKGRGPLFSLSKVTSVDSIGVAYLAFKDKVYFNVTTHSTSYKDIQVNGGELILIHQNPGGFCAY

SEQ ID NO: 808 - DNA, 1266 bp, synthetic construct
  source 1..1266: mol_type = other DNA
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaaaggagcccccagccccagcccaagccccagcccaagcccgagcccgagggcagcctgcacgcctgcggctgcgccgccgcccccgacatcaaggacctgctgagcaggctggaggagctggagggcctggtgagcagcctgagggagcagggcaccggcggcggcagcggcggcaggggcctgcagcaggtgccgcctcagtatcctccaattcaaagtatcagagtacaatttaccaggtgtgagaatgagaaaggttgcatcatcacatccccaagcaaggatgaaactatgaaggtgcaagacaactcaatcatcattaactgtgatgggttttatctcatctccctgaagggttacttctctgaggagctcagcctcagcctttattaccgaaagggtcggggacccctcttctctctgagcaaggtcacatctgttgactccattggagtggcctatctggctttcaaggacaaagtctactttaatgtgaccactcacagtacctcctacaaagacatccaggtgaatggtggggaattgattctcattcatcaaaatcctggtggcttctgtgcctac

SEQ ID NO: 809 - protein, 422 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRSPQPQPKPQPKPEPEGSLHACGCAAAPDIKDLLSRLEELEGLVSSLREQGTGGGSGGRGLQQVPPQYPPIQSIRVQFTRCENEKGCIITSPSKDETMKVQDNSIIINCDGFYLISLKGYFSEELSLSLYYRKGRGPLFSLSKVTSVDSIGVAYLAFKDKVYFNVTTHSTSYKDIQVNGGELILIHQNPGGFCAY

SEQ ID NO: 810 - DNA, 567 bp, synthetic construct
  source 1..567: mol_type = other DNA
caccatcaccatcaccatggccccggccaggtgcagctgcacgcctgcggctgcgccgccgcccccgacatcaaggacctgctgagcaggctggaggagctggagggcctggtgagcagcctgagggagcagggcaccggcggcggcagcggcggcaggggcctgcagcaggtgccgcctcagtatcctccaattcaaagtatcagagtacaatttaccaggtgtgagaatgagaaaggttgcatcatcacatccccaagcaaggatgaaactatgaaggtgcaagacaactcaatcatcattaactgtgatgggttttatctcatctccctgaagggttacttctctgaggagctcagcctcagcctttattaccgaaagggtcggggacccctcttctctctgagcaaggtcacatctgttgactccattggagtggcctatctggctttcaaggacaaagtctactttaatgtgaccactcacagtacctcctacaaagacatccaggtgaatggtggggaattgattctcattcatcaaaatcctggtggcttctgtgcctac

SEQ ID NO: 811 - protein, 189 aa, synthetic construct
HHHHHHGPGQVQLHACGCAAAPDIKDLLSRLEELEGLVSSLREQGTGGGSGGRGLQQVPPQYPPIQSIRVQFTRCENEKGCIITSPSKDETMKVQDNSIIINCDGFYLISLKGYFSEELSLSLYYRKGRGPLFSLSKVTSVDSIGVAYLAFKDKVYFNVTTHSTSYKDIQVNGGELILIHQNPGGFCAY